(19) United States
(12) Patent Application Publication
(10) Pub. No.: US 2025/0361230 A1
(43) Pub. Date: Nov. 27, 2025

(54) LATS Inhibitors and Uses Thereof

(71) Applicant: The USA, as Represented by the Secretary, Dept. of Heath and Human Services, Bethesda, MD (US)

(72) Inventors: Patrick J. Morris, Bethesda, MD (US); Damien Y. Duveau, Bethesda, MD (US); Craig J. Thomas, Bethesda, MD (US); Scott B. Hoyt, Bethesda, MD (US); Michele Ceribelli, Bethesda, MD (US)

(21) Appl. No.: 18/872,732

(22) PCT Filed: Jun. 6, 2023
(86) PCT No.: PCT/US2023/024589

§ 371 Date: Dec. 6, 2024

Related U.S. Application Data
(60) Provisional application No. 63/349,337, filed on Jun. 6, 2022.

(51) Int. Cl. C07D 471/04; A61K 31/437; A61K 31/439; A61K 31/4439; A61K 31/444; A61K 31/4545; A61K 31/4725; A61K 31/5377; A61K 31/675; C07D 519/00; C07F 9/6561

(52) U.S. Cl. CPC C07D 471/04; A61K 31/437; A61K 31/439; A61K 31/4439; A61K 31/444; A61K 31/4545; A61K 31/4725; A61K 31/5377; A61K 31/675; C07D 519/00; C07F 9/6561

(57) ABSTRACT

The present disclosure provides certain LATS1 and/or LATS2 inhibitors, including selective LATS1/LATS2 inhibitors and dual LATS1/LATS2 and AKT inhibitors that are useful for the treatment of wounds, of diseases that would benefit from organ or cellular regeneration, of cancer, and of heavy metal poisoning, as well as for promoting ex vivo growth of a cell line or a cell product and for accelerating tissue growth ex vivo. Also provided are pharmaceutical compositions containing such compounds and methods of using such compounds.

### CROSS REFERENCE TO RELATED APPLICATIONS

This application claims priority to U.S. Provisional Patent Application No. 63/349,337, filed Jun. 6, 2022, the contents of which are hereby incorporated by reference in their entirety.

### BACKGROUND OF THE INVENTION

### LATS1/LATS2 Inhibition

The Hippo pathway is a critical cellular pathway which regulates organ size control, tissue regeneration, and self-renewal (Boopathy and Hong, Frontiers in Cell Development and Biology, 2019, doi.org/10.3389/fcell.2019.00049). Within the Hippo pathway are a pair of canonical serine/threonine kinases, LATS1 (Large Tumor Suppressor Kinase 1) and LATS2 (Large Tumor Suppressor Kinase 2). When the Hippo pathway is activated, the upstream kinases MST1 and MST2 phosphorylate LATS1 and LATS2, which then phosphorylate YAP (Yes Associated Protein), resulting in its retention in the cytoplasm and degradation. By contrast, when the Hippo pathway is turned off, LATS1/2 are not phosphorylated, and these enzymes do not phosphorylate YAP. This leads to YAP localizing to the nucleus, complexing with TEAD, and activating the expression of downstream target genes that promote cell growth and proliferation. The Hippo pathway can be intercepted at LATS1/2 by genetic or pharmacological interventions which remove LATS1/2 or inhibit the activity of LATS1/2, resulting in YAP localizing to the nucleus and activating the cell growth and proliferation cycle. (Dey et al, Nature Reviews Drug Discovery, 2020, doi.org/10.1038/s41573-020-0070-z). Indeed, genetic knockout experiments on LATS1 and LATS2 demonstrated that loss of LATS1/2 in adult murine livers caused rapid expansion of immature biliary epithelial cells (Lee, et al, Nature Communications, 2015, doi.org/10.1038/ncomms1961). Thus, inhibition of LATS1/2 is a promising target for pharmacological intervention in order to activate native cell growth and proliferation, and to prompt cellular expansion and regeneration.

Therapeutics that can safely and effectively promote organ regeneration, repair, and accelerated healing remain a broad unmet need within the biochemical community. In the United States today, there are major shortages in the number of organs needed for transplant into heathy humans, with over 10,000 people currently on the waiting list for a liver transplant, and more than 90,000 people on the waiting list for a kidney transplant (www.organdonor.gov, 2020). In addition, chronic skin wounds remain a major medical burden within the United States, with more than 6.5 million patients in 2010 (Sen, et al. Wound Repair and Regeneration, 2010, doi: 10.1111/j.1524-475X.2009.00543.x).

While the liver is typically able to regenerate quite well, upon sufficient injury it can lose its ability to regenerate, and the regenerative hepatocytes instead can become apoptotic or senescent. Upon such an event, fibrosis occurs, leading to cirrhosis and hepatocellular carcinoma (Campana et al. Nature Reviews Molecular Cell Biology, 2021, doi.org/10.1038/s41580-021-00373-7). Thus, there is a clear need for a regenerative therapeutic with the ability to reverse the senescence of the regenerative hepatocytes, allowing for the liver to regain its regenerative capabilities and restore its function, and accelerating liver repair.

When damaged, the kidneys are an organ capable of regeneration. However, the process is often incomplete, leading to partial or limited functionality. Additionally, when the kidneys are damaged, supporting dialysis is often required by the body while the regeneration process occurs (Reule and Gupta, Organogenesis, 2011, doi.org/10.4161/org.7.2.16285). A therapeutic capable of accelerating regeneration of the kidney could reduce the required time on dialysis and potentially reduce the critical need for organ transplants.

Wound healing within the epidermis and dermis remains an area of medical need. This need relates to the repair of acute wounds, such as surgical wounds, bites, and traumatic injury, but also relates to chronic wounds, including foot ulcers, pressure sores, and other chronic wounds that are often associated with metabolic diseases such as diabetes and other chronic conditions (Okur et. al. Asian Journal of Pharmaceutical Sciences. 2020, doi.org/10.1016/j.ajps.2019.11.008). Several studies have demonstrated that downregulation/deletion of YAP1 in mice has a very detrimental role in skin regeneration. (Dey et al, Nature Reviews Drug Discovery, 2020, doi.org/10.1038/s41573-020-0070-z). Conversely, upregulation of YAP1 via inhibition of LATS represents a promising pathway for the acceleration of skin regeneration.

Development of a small molecule inhibitor of LATS1 and LATS2 would have profound potential capabilities in wound healing and organ regeneration. Effectively turning off the Hippo pathway allows for the return of native cell growth, proliferation, and regenerative capabilities for these cells. This would ideally accelerate the native regenerative capacity of the injured organs and allow them to return to a functioning capacity. With faster, more extensive regeneration would come a reduction in the need for additional transplants, removal of the need for excessive dialysis, and a reduction in the time for wounds to heal. Additionally, there are several other organ systems which may potentially be beneficially served by advanced acceleration in their wound healing. Inhibitors that selectively inhibit LATS1/2 over AKT may be of particular benefit in wound healing and organ regeneration applications, by minimizing the anti-cell proliferative effects of AKT inhibition.

In addition, in ex vivo cellular cultures, the inhibition of inherent LATS1/2 activity may be able to speed the reproduction of critical, slow growing cell lines. These may include stem cell lines designed for human therapeutics, slow growing research lines, and potentially alternative cell lines designed for more industrial purposes.

### Dual Inhibition of LATS1/LATS2 and AKT

AKT, also known as protein kinase B is a serine/threonine protein kinase within the PI3K/AKT/mTOR signaling pathway that plays key roles in multiple cellular processes, especially in regards to cell proliferation, transcription, and cell migration (Martorana, et al. Front. Pharmacol. 2021. doi.org/10.3389/fphar.2021.662232). AKT has a pronounced role in many cancers, as it is capable of blocking apoptosis and is overexpressed in several different cancer lines (Hill, et al. Pharmacology and Therapeutics. 2002. doi:10.1016/S0163-7258(02)00193-6). Due to these roles in oncology, the development of AKT inhibitors to treat several different types of cancer has been a priority, with two phase III trials for the AKT inhibitors capivasertib and ipatasertib currently underway (Martorana, et al. Front. Pharmacol. 2021. doi.org/10.3389/fphar.2021.662232).

Despite inhibition of LATS1/2 triggering cell growth and proliferation, certain cancer cell lines appear to depend on LATS1/2 for growth and proliferation. Pan and coworkers demonstrated in 2018 that the growth and proliferation of the MC38 colon adenocarcinoma line depended on LATS1 being active. When the gene was knocked out, the growth and proliferation of the cancer line was sharply attenuated. (Pan et al. Oncogene, 2018, DOI: 101038/s41388-018-0610-8). In certain other systems, LATS1/2 appeared to play a major role in providing resistance to chemotherapy agents (Moroishi et al, Cell, 2016, DOI: 10.1016/j.cell.2016.11.005). Thus direct inhibition of LATS1/2 may prove useful as a method of treatment for select cancers. Combining a LATS1/2 inhibitor with an AKT inhibitor may yield a complimentary effect with a particular benefit in the treatment of certain cancers.

Development of a small molecule inhibitor of LATS1/2 combined with AKT may have profound potential capabilities in the area of oncology, wound regeneration and organ regeneration. By effectively turning off the Hippo pathway, oncology lines that depend on LATS1/2 activity would be impaired.

### SUMMARY OF THE INVENTION

In a first aspect, the disclosure provides a compound of Formula I.

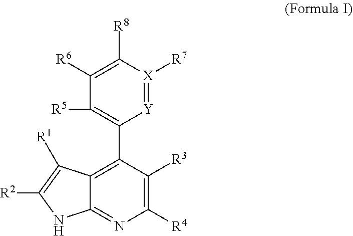

or a pharmaceutically acceptable salt thereof,
wherein
X and Y are independently C or N;
R¹ is —H, halo, —CN, —C₁-C₆ alkyl, —C₁-C₆ alkenyl, —C(O)O—C₁-C₆ alkyl, a 6-10 membered aryl ring, or a 5-10 membered heteroaryl ring containing 1, 2, or 3 heteroatoms, wherein the aryl or heteroaryl ring is optionally substituted with one or more substituents selected from halo, —C₁-C₆ alkyl, —O—C₁-C₆ alkyl, halogenated C₁-C₆ alkyl, and —C(O)NH₂;
R² is —H or —C₁-C₆ alkyl;
R³ is —H or halo;
R⁴ is —H, halo, —C₁-C₆ alkyl, —NR^{a}R^{b}, or a 5-10 membered heteroaryl ring containing 1 or 2 heteroatoms;
R⁵ is —H, halo, —CN, —C₁-C₆ alkyl, halogenated C₁-C₆ alkyl, or —O—C₁-C₆ alkyl, wherein —O—C₁-C₆ alkyl is optionally substituted with one or more halo groups;
R⁶ is —H, halo, —C₁-C₆ alkyl, —C₁-C₆ alkenyl, —C₁-C₆ alkynyl, —NR^{c}R^{d}, phenyl, pyridyl, or —O—C₁-C₆ alkyl, wherein —C₁-C₆ alkyl is optionally substituted with one or more halo groups, wherein —C₁-C₆ alkynyl is optionally substituted with one or more hydroxyl groups, and wherein phenyl is optionally substituted with one or more substituents selected from aminoalkyl and amide;
R⁷ is absent, —H, halo, or —C₁-C₆ alkyl;
R⁸ is

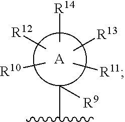

or R⁸ is

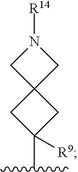

or R⁸ is —C(R^{e})(R^{f})N(R^{g})(R^{h}) or —C(O)NH₂;
or R⁸ and R⁶ together with the carbons to which they are attached form a 5- or 6-membered ring containing 1 or 2 heteroatoms and optionally a carbonyl group;
R^{a} and R^{b} are independently selected from —H and —C₁-C₆ alkyl;
R^{c} and R^{d} are independently selected from —H, —C₁-C₆ alkyl, and alkoxyalkyl;
R^{e} and R^{f} are independently selected from —H and —C₁-C₆ alkyl;
R^{g} is —H or —C₁-C₆ alkyl;
R^{h} is —H, —C₁-C₆ alkyl, —C(O)—C₁-C₆ alkyl, or arylalkyl, wherein the aryl group of arylalkyl is optionally substituted with halo or —OCF₃;
or R^{g} and R^{h} together with the carbons to which they are attached form a 5 or 6 membered heterocyclic ring;
A is a 4-6 membered heterocycloalkyl ring containing 1 nitrogen, optionally substituted at any position by one or more of R, R, R¹², R¹³, and R¹⁴;
R⁹ is —H, —OH, —C₁-C₆ alkyl, halogenated C₁-C₆ alkyl, —COOH, —C(O)O—C₁-C₆ alkyl, or a 5- or 6-membered ring containing 1, 2, or 3 heteroatoms, wherein the ring is optionally substituted with one or more —C₁-C₆ alkyl groups;
R¹⁰ and R¹¹ are independently absent, —H or —C₁-C₆ alkyl, or R¹⁰ and R¹¹ together form a —CH₂— or —CH₂CH₂— bridge between the atoms to which they are attached to form an azabicyclo group with ring A;
R¹² and R¹³ are independently absent, —H or —C₁-C₆ alkyl, or R¹² and R¹³ together form a —CH₂—, —CH₂CH₂—, or —CH₂CH₂CH₂— bridge between the atoms to which they are attached to form an azabicyclo group with ring A;
R¹⁴ is —H, —C₁-C₆ alkyl, —C(O)—R, —C(O)O—C₁-C₆ alkyl, alkoxyalky, a 3-6 membered cycloalkyl ring, a 3-6 membered heterocycloalkyl ring having 1 heteroatom, a 5-9 membered spiro heterocycloalkyl group having 1 heteroatom, heteroarylalkyl, —S⁺(O—)(O)—CH₂CH₂—R^{j}, or

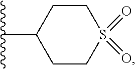 [SMILES: *C1CCS(=O)(=O)CC1]

wherein —C₁-C₆ alkyl is optionally substituted with one or more halo groups;
or R¹² and R¹⁴ together with the atoms to which they are attached form a 4-6 membered cycloalkyl ring;
Rⁱ is —H, —C₁-C₆ alkyl, —C₁-C₆ alkyl-NR^{k}R^{k′}, a 3-5 membered cycloalkyl ring, or a 3-6 membered heterocycloalkyl ring having 1 or 2 heteroatoms, wherein the alkyl of —C₁-C₆ alkyl-NR^{k}R^{k′} is optionally substituted with one or more of halo or —OH, and wherein the cycloalkyl or heterocycloalkyl ring is optionally substituted with one or more halo, —CF₃, —C₁-C₆ alkyl, —OH, oxo, or amine groups;
R^{j} is —N(CH₃)₂, —Si(CH₃)₃, a 3-5 membered cycloalkyl ring, or alkoxy; and
R^{k} and R^{k′} are each independently —H or —C₁-C₆ alkyl, wherein —C₁-C₆ alkyl is optionally substituted with one or more halo groups.

In a second aspect, the disclosure provides a compound of Formula II

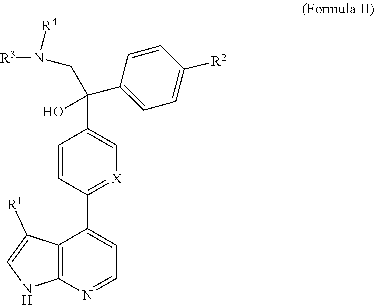

or a pharmaceutically acceptable salt thereof,
wherein
X is C or N;
R¹ is —H, halo, a 6-10 membered aryl ring, a 5-10 membered heteroaryl ring containing 1 or 2 heteroatoms, or —C(O)O—C₁-C₆ alkyl;
R² is —H or halo;
R³ is —H or —C₁-C₆ alkyl; and
R⁴ is —H, —C(O)—R^{a}, or

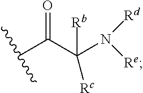

R^{a} is a 4-6 membered heteroaryl ring containing 1 or 2 heteroatoms, or —C₁-C₆ alkyl, wherein the —C₁-C₆ alkyl is optionally substituted with one or more of —OH, —P(O)(OH)₂;
R^{b} and R^{c} are independently selected from —H and —C₁-C₆ alkyl;
R^{d} is —H or —C₁-C₆ alkyl; and
R^{e} is —H, —C₁-C₆ alkyl, or —C(O)—CH₂NH₂.

In a third aspect, the disclosure provides a pharmaceutical composition comprising a compound of the first aspect of the invention and a pharmaceutically acceptable carrier, excipient, or diluent.

In a fourth aspect, the disclosure provides a pharmaceutical composition comprising a compound of the second aspect of the invention and a pharmaceutically acceptable carrier, excipient, or diluent.

In a fifth aspect, the disclosure provides a method of inhibiting one or more of large tumor suppressor kinase 1 (LATS1) and large tumor suppressor kinase 2 (LATS2) in a subject in need thereof, comprising administering to the subject a compound of the first aspect of the invention or the pharmaceutical composition of the third aspect of the invention.

In a sixth aspect, the disclosure provides a method of treating, preventing, or ameliorating a disease, disorder, or condition associated with LATS1 or LATS2 in a subject in need thereof comprising administering to the subject a therapeutically effective amount of a compound of the first aspect of the invention or the pharmaceutical composition of the third aspect of the invention.

In a seventh aspect, the disclosure provides a method of promoting ex vivo growth of a cell line or of a cell product comprising exposing the cell line or cell product to a compound of the first aspect of the invention or the pharmaceutical composition of the third aspect of the invention.

In an eighth aspect, the disclosure provides a method of accelerating tissue growth ex vivo comprising exposing the tissue to a compound of the first aspect of the invention or the pharmaceutical composition of the third aspect of the invention.

In a ninth aspect, the disclosure provides a method of jointly inhibiting protein kinase B (AKT) and one or more of large tumor suppressor kinase 1 (LATS1) and large tumor suppressor kinase 2 (LATS2) in a subject in need thereof, comprising administering to the subject a compound of the second aspect of the invention or the pharmaceutical composition of the fourth aspect of the invention.

In a tenth aspect, the disclosure provides a method of treating, preventing, or ameliorating a disease, disorder, or condition associated with LATS1, LATS2, or AKT in a subject in need thereof comprising administering to the subject a therapeutically effective amount of a compound of the second aspect of the invention or the pharmaceutical composition of the fourth aspect of the invention.

In an eleventh aspect, the disclosure provides a method of promoting ex vivo growth of a cell line or of a cell product comprising exposing the cell line or cell product to a compound of the second aspect of the invention or the pharmaceutical composition of the fourth aspect of the invention.

In a twelfth aspect, the disclosure provides a method of accelerating tissue growth ex vivo comprising exposing the tissue to a compound of the second aspect of the invention or the pharmaceutical composition of the fourth aspect of the invention.

### DETAILED DESCRIPTION OF THE INVENTION

The articles “a” and “an” are used herein to refer to one or to more than one (i.e., to at least one) of the grammatical object of the article. By way of example, “an element” means one element or more than one element.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” or the like are to be understood to be open-ended, i.e., to mean including but not limited to. Only the transitional phrases “consisting of” and “consisting essentially of” shall be closed or semi-closed transitional phrases, respectively. The open-end phrases such as “comprising” include and encompass the close-ended phrases.

Where ranges are provided, it is to be understood that the range refers not just to the specified range, but that it also encompasses any sub-range of or any single value within the recited range, even if not specifically recited.

The definition of each expression, e.g., alkyl, or the like, when it occurs more than once in any structure, is intended to be independent of its definition elsewhere in the same structure.

### Definitions

Unless otherwise stated, the following terms used in the specification and claims are defined for the purposes of this application and have the following meaning

As used herein, “alkenyl” refers to hydrocarbon chains of either straight or branched configuration having the specified number of carbon atoms and one or more, preferably one to two, carbon-carbon double bonds that may occur in any stable point along the chain.

As used herein, “alkoxy” is an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, “alkoxyalkyl” is a branched or unbranched aliphatic radical containing the indicated number of carbon atoms with at least one alkoxy group as defined herein.

As used herein, “alkyl” is a branched or unbranched aliphatic radical containing the indicated number of carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

As used herein, “alkynyl” refers to hydrocarbon chains of either straight or branched configuration having one or more, preferably one to three, carbon-carbon triple bonds that may occur in any stable point along the chain.

As used herein, “ameliorate” with respect to the administration of the compounds of the invention refers to bettering a disease, disorder, or condition disclosed herein, including alleviating symptoms thereof.

As used herein, “amide” refers to an amino linked to a carbonyl group.

As used herein, the term “amine” or “amino” means —NR′R″, wherein R′ and R″ are independently —H, alkyl, or another moiety.

As used herein, “aminoalkyl” refers to an alkyl group on which one of the hydrogen atoms is replaced by an amino group.

As used herein, the term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicyclic aromatic rings. For example, bicyclic aromatic rings containing heteroatoms in a hydrocarbon ring structure are referred to as bicyclic heteroaryl rings.

As used herein, “aryl” is an aromatic cyclic (monocyclic or polycyclic) group containing only carbon ring atoms such as a phenyl group or a naphthyl group.

As used herein, “arylalkyl” is an acyclic alkyl radical in which one of the hydrogen atoms bonded to a carbon atom, is replaced with an aryl radical.

As used herein, “bridge” or “bridged” refers to a ring system where one or more atoms (i.e., C, O, N, or S) link two non-adjacent carbon or nitrogen atoms of the ring.

As used herein, “carbonyl group” means a ═(O) group.

As used herein, the term “cyclic” pertains to compounds and/or groups which have one ring, or two or more rings (e.g., spiro, fused, bridged).

As used herein, “cycloalkyl” is a saturated or partially unsaturated monocyclic or polycyclic ring group. Examples of cycloalkyl groups include cyclopropyl, cyclobutyl, cyclopentyl, and cyclohexyl.

As used herein, a “disease, disorder, or condition associated with LATS1 or LATS2” is a disease, disorder, or condition for which LATS 1 and/or LATS 2 is implicated or has a role in the development or maintenance of the disease, disorder, or condition, whether directly or indirectly, and/or for which inhibition of LATS1 and/or LATS2 may be beneficial. Similarly, a “disease, disorder, or condition associated with LATS1, LATS2, or AKT” is a disease, disorder, or condition for which LATS 1 and/or LATS 2 and/or AKT is implicated or has a role in the development or maintenance of the disease, disorder, or condition, whether directly or indirectly, and/or for which inhibition of LATS1 and/or LATS2 and/or AKT may be beneficial.

As used herein, “halo” or “halogen” means Cl, Br, I or F.

As used herein, “heteroaryl” refers to an aromatic cyclic group having one, two, or more fused rings where at least one ring is aromatic, and containing 1, 2, or 3 heteroatoms independently chosen from N, O, and S. Monocyclic heteroaryl groups typically have from 5 to 7 ring atoms. Examples of heteroaryl groups include, but are not limited to, oxazolyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, and isoxazolyl.

As used herein, “heteroarylalkyl” refers to an acyclic alkyl radical in which one of the hydrogen atoms bonded to a carbon atom, is replaced with a heteroaryl radical.

As used herein, a “heteroatom” is an atom of any element other than carbon or hydrogen. Illustrative heteroatoms include boron, nitrogen, oxygen, phosphorus, sulfur and selenium.

As used herein, “heterocycloalkyl” or “heterocyclic” means a saturated or partially unsaturated monocyclic or polycyclic ring group containing carbon atoms and 1 or more ring atoms independently chosen from N, O, and S. Examples of heterocycloalkyl groups includes azepines, azetidinyl, morpholinyl, pyranyl, oxopiperidinyl, oxopyrrolidinyl, piperazinyl, piperidinyl, pyrrolidinyl, quinicludinyl, thiomorpholinyl, tetrahydropyranyl and tetrahydrofuranyl.

As used herein, “optionally substituted” means a moiety as defined herein that is substituted as defined herein with zero or one or more non-hydrogen groups.

As used herein, “oxo” refers to a carbonyl group as defined herein.

As used herein, a “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference in its entirety.

As used herein, “prevent” or “preventing” refers to prophylactic treatment, i.e. administering an amount of the LATS1/2 inhibitors disclosed herein effective to significantly reduce the chance developing a disease, disorder, or condition as referenced herein.

As used herein, the term “saturated” pertains to compounds and/or groups which do not have any carbon-carbon double bonds or carbon-carbon triple bonds.

As used herein, “spiro” refers to a ring system in which at least two rings have only one common atom.

As used herein, “substituted” means that means that at least one hydrogen atom (attached to a carbon atom or heteroatom) is replaced with a non-hydrogen group. When a substituent is oxo (i.e., ═O), then 2 hydrogens on the atom are replaced. It will be understood that “substituted” includes the implicit proviso that such substitution is in accordance with permitted valence of the substituted atom and the substituent, and that the substitution results in a stable compound, e.g., a compound which does not spontaneously undergo transformation such as by rearrangement, cyclization, elimination, or other reaction. The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. This disclosure is not intended to be limited in any manner by the permissible substituents of organic compounds. “—H” is not considered a substituent.

As used herein, a “therapeutic product” is a composition or formulation used for therapeutic purposes, i.e. in the treatment, prevention, or amelioration of a disease, disorder, or condition in a subject in need thereof.

As used herein, a “therapeutically effective amount” or “effective amount” is the amount of a compound of the present disclosure and/or a pharmaceutically acceptable salt thereof that, when administered to a subject, is sufficient to achieve a pharmacological effect or therapeutic improvement, preferably without undue adverse side effects. The “therapeutically effective amount” can vary depending on the compound, the disease, disorder, or condition and its severity and the age, weight, etc., of the subject to be treated.

As used herein, the terms “treat,” “treating,” and “treatment” mean implementation of therapy with the intention of reducing in severity or frequency symptoms, elimination of symptoms or underlying cause, prevention of the occurrence of symptoms or their underlying cause, or the improvement or remediation of damage due to a disease, disorder, or condition.

As used herein, the term “unsaturated” pertains to compounds and/or groups which have at least one carbon-carbon double bond or carbon-carbon triple bond.

The terms as defined above are also intended to include variations of the terms as would be used and understood by one of skill in the art. In a non-limiting example, “substituted” as defined herein refers not only to “substituted,” but also to “substitution,” “substituted with,” and the like.

### COMPOUNDS

In a first aspect, the disclosure provides a compound of Formula I.

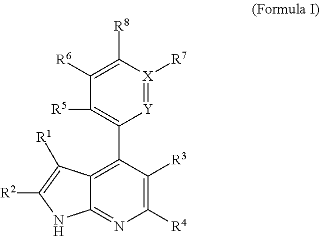

or a pharmaceutically acceptable salt thereof,
wherein
X and Y are independently C or N;
R¹ is —H, halo, —CN, —C₁-C₆ alkyl, —C₁-C₆ alkenyl, —C(O)O—C₁-C₆ alkyl, a 6-10 membered aryl ring, or a 5-10 membered heteroaryl ring containing 1, 2, or 3 heteroatoms, wherein the aryl or heteroaryl ring is optionally substituted with one or more substituents selected from halo, —C₁-C₆ alkyl, —O—C₁-C₆ alkyl, halogenated C₁-C₆ alkyl, and —C(O)NH₂;
R² is —H or —C₁-C₆ alkyl;
R³ is —H or halo;
R⁴ is —H, halo, —C₁-C₆ alkyl, —NR^{a}R^{b}, or a 5-10 membered heteroaryl ring containing 1 or 2 heteroatoms;
R⁵ is —H, halo, —CN, —C₁-C₆ alkyl, halogenated C₁-C₆ alkyl, or —O—C₁-C₆ alkyl, wherein —O—C₁-C₆ alkyl is optionally substituted with one or more halo groups;
R⁶ is —H, halo, —C₁-C₆ alkyl, —C₁-C₆ alkenyl, —C₁-C₆ alkynyl, —NR^{c}R^{d}, phenyl, pyridyl, or —O—C₁-C₆ alkyl, wherein —C₁-C₆ alkyl is optionally substituted with one or more halo groups, wherein —C₁-C₆ alkynyl is optionally substituted with one or more hydroxyl groups, and wherein phenyl is optionally substituted with one or more substituents selected from aminoalkyl and amide;
R⁷ is absent, —H, halo, or —C₁-C₆ alkyl; R⁸ is

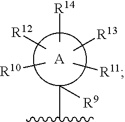

or R⁸ is

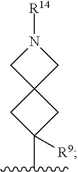

or R⁸ is —C(R′)(R^{f})N(R^{g})(R^{h}) or —C(O)NH₂;
or R⁸ and R⁶ together with the carbons to which they are attached form a 5- or 6-membered ring containing 1 or 2 heteroatoms and optionally a carbonyl group;
R^{a} and R^{b} are independently selected from —H and —C₁-C₆ alkyl;
R^{c} and R^{d} are independently selected from —H, —C₁-C₆ alkyl, and alkoxyalkyl;
R^{e} and R^{f} are independently selected from —H and —C₁-C₆ alkyl;
R^{g} is —H or —C₁-C₆ alkyl;
R^{h} is —H, —C₁-C₆ alkyl, —C(O)—C₁-C₆ alkyl, or arylalkyl, wherein the aryl group of arylalkyl is optionally substituted with halo or —OCF₃;
or R^{g} and R^{h} together with the carbons to which they are attached form a 5 or 6 membered heterocyclic ring;
A is a 4-6 membered heterocycloalkyl ring containing 1 nitrogen, optionally substituted at any position by one or more of R¹⁰, R, R¹², R¹³, and R¹⁴;
R⁹ is —H, —OH, —C₁-C₆ alkyl, halogenated C₁-C₆ alkyl, —COOH, —C(O)O—C₁-C₆ alkyl, or a 5- or 6-membered ring containing 1, 2, or 3 heteroatoms, wherein the ring is optionally substituted with one or more —C₁-C₆ alkyl groups;
R¹⁰ and R¹¹ are independently absent, —H or —C₁-C₆ alkyl, or R¹⁰ and R¹¹ together form a —CH₂— or —CH₂CH₂— bridge between the atoms to which they are attached to form an azabicyclo group with ring A;
R¹² and R¹³ are independently absent, —H or —C₁-C₆ alkyl, or R¹² and R¹³ together form a —CH₂—, —CH₂CH₂—, or —CH₂CH₂CH₂— bridge between the atoms to which they are attached to form an azabicyclo group with ring A;
R¹⁴ is —H, —C₁-C₆ alkyl, —C(O)—R, —C(O)O—C₁-C₆ alkyl, alkoxyalky, a 3-6 membered cycloalkyl ring, a 3-6 membered heterocycloalkyl ring having 1 heteroatom, a 5-9 membered spiro heterocycloalkyl group having 1 heteroatom, heteroarylalkyl, —S⁺(O—)(O)—CH₂CH₂—R^{j}, or

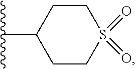 [SMILES: *C1CCS(=O)(=O)CC1]

wherein —C₁-C₆ alkyl is optionally substituted with one or more halo groups;
or R¹² and R¹⁴ together with the atoms to which they are attached form a 4-6 membered cycloalkyl ring;
Rⁱ is —H, —C₁-C₆ alkyl, —C₁-C₆ alkyl-NR^{k}R^{k′}, a 3-5 membered cycloalkyl ring, or a 3-6 membered heterocycloalkyl ring having 1 or 2 heteroatoms, wherein the alkyl of —C₁-C₆ alkyl-NR^{k}R^{k′} is optionally substituted with one or more of halo or —OH, and wherein the cycloalkyl or heterocycloalkyl ring is optionally substituted with one or more halo, —CF₃, —C₁-C₆ alkyl, —OH, oxo, or amine groups;
R^{j} is —N(CH₃)₂, —Si(CH₃)₃, a 3-5 membered cycloalkyl ring, or alkoxy; and
R^{k} and R^{k′} are each independently —H or —C₁-C₆ alkyl, wherein —C₁-C₆ alkyl is optionally substituted with one or more halo groups.

In one embodiment of the first aspect of the invention, the compound is a compound of Formula IA

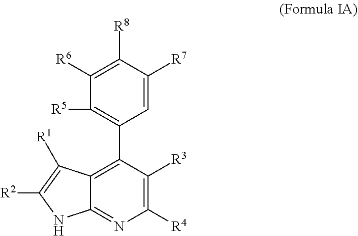

or a pharmaceutically acceptable salt thereof, wherein the R groups are as defined for Formula I.

In another embodiment of the first aspect of the invention, the compound is a compound of Formula IB

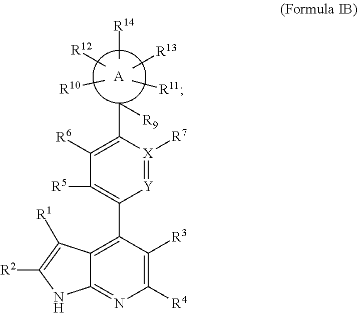

or a pharmaceutically acceptable salt thereof, wherein the R groups are as defined for Formula I.

In another embodiment of the first aspect of the invention, the compound is a compound of Formula IC

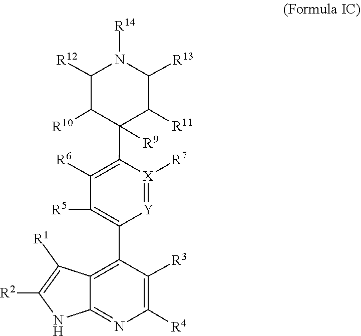

or a pharmaceutically acceptable salt thereof, wherein the R groups are as defined for Formula I.

In another embodiment of the first aspect of the invention, the compound is a compound of Formula ID

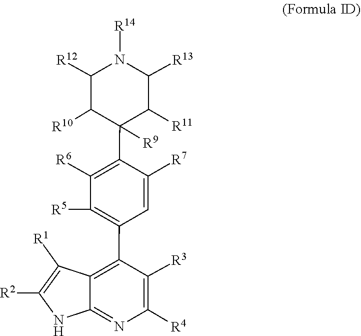

or a pharmaceutically acceptable salt thereof, wherein the R groups are as defined for Formula I.

In another embodiment of the first aspect of the invention, the compound is a compound of Formula IE

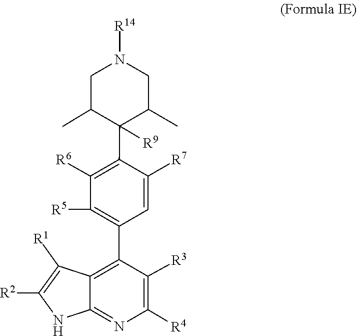

or a pharmaceutically acceptable salt thereof, wherein the R groups are as defined for Formula I.

In another embodiment of the first aspect of the invention, the compound is a compound of Formula IF

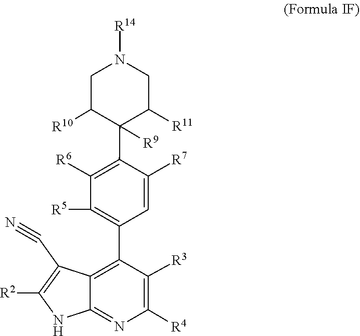

or a pharmaceutically acceptable salt thereof, wherein the R groups are as defined for Formula I.

In another embodiment of the first aspect of the invention, R¹ is —H, halo, —CN, or —C(O)O—C₁-C₆ alkyl.

In another embodiment of the first aspect of the invention, R¹ is —H.

In another embodiment of the first aspect of the invention, R¹ is halo.

In another embodiment of the first aspect of the invention, R¹ is —F or —Br.

In another embodiment of the first aspect of the invention, R¹ is —CN.

In another embodiment of the first aspect of the invention, R¹ is —C(O)O—C₁-C₆ alkyl.

In another embodiment of the first aspect of the invention, R¹ is —CO₂Me.

In another embodiment of the first aspect of the invention, R¹ is

—H;
—F;
—Br;
—Cl;
—CN;
—CH₃;

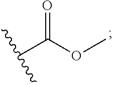 [SMILES: *C(=O)OC]

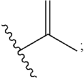 [SMILES: *C(=C)C]

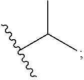 [SMILES: *C(C)C]

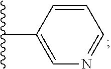 [SMILES: *c1cccnc1]

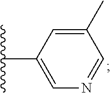 [SMILES: *c1cncc(C)c1]

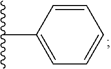 [SMILES: *c1ccccc1]

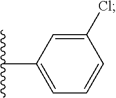 [SMILES: *c1cccc(Cl)c1]

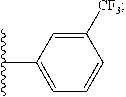 [SMILES: *c1cccc(C(F)(F)F)c1]

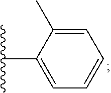 [SMILES: *c1ccccc1C]

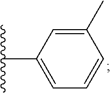 [SMILES: *c1cccc(C)c1]

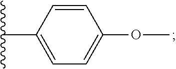 [SMILES: *c1ccc(OC)cc1]

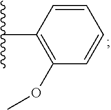 [SMILES: *c1ccccc1OC]

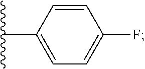 [SMILES: *c1ccc(F)cc1]

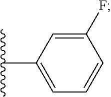 [SMILES: *c1cccc(F)c1]

or

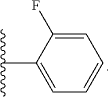 [SMILES: *c1ccccc1F]

In another embodiment of the first aspect of the invention, R² is —H or —CH₃.

In another embodiment of the first aspect of the invention, R³ is

| | | |
|---|---|---|
| —H; | —F; | or —Cl. |

In another embodiment of the first aspect of the invention, R³ is —F or —Cl.

In another embodiment of the first aspect of the invention, R⁴ is —H, halo, —C₁-C₆ alkyl, or —NR^{a}R^{b}.

In another embodiment of the first aspect of the invention, R⁴ is —H.

In another embodiment of the first aspect of the invention, R⁴ is halo.

In another embodiment of the first aspect of the invention, R⁴ is —Cl.

In another embodiment of the first aspect of the invention, R⁴ is —C₁-C₆ alkyl.

In another embodiment of the first aspect of the invention, R⁴ is —CH₃.

In another embodiment of the first aspect of the invention, R⁴ is —NR^{a}R^{b}.

In another embodiment of the first aspect of the invention, R⁴ is —NH₂.

In another embodiment of the first aspect of the invention, R⁴ is

—H;
—Cl;
—CH₃;
—NH₂;
or

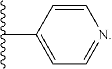 [SMILES: *c1ccncc1]

In another embodiment of the first aspect of the invention, R⁵ is

| | | | |
|---|---|---|---|
| —H; | —F; | —Cl; | —CH₃; |
| —CF₃; | —CN; | —OCH₃; | or —OCF₃. |

In another embodiment of the first aspect of the invention, R⁵ is —H.

In another embodiment of the first aspect of the invention, R⁶ is halo or —C₁-C₆ alkyl.

In another embodiment of the first aspect of the invention, R⁶ is halo.

In another embodiment of the first aspect of the invention, R⁶ is —Cl or —F.

In another embodiment of the first aspect of the invention, R⁶ is —C₁-C₆ alkyl.

In another embodiment of the first aspect of the invention, R⁶ is —CH₃.

In another embodiment of the first aspect of the invention, R⁶ is

—H;
—F;
—Cl;
—CH₃;
—CF₃;
—OCH₃;
—OCH₂CH₃;

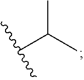 [SMILES: *C(C)C]

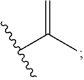 [SMILES: *C(=C)C]

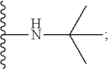 [SMILES: *NC(C)(C)C]

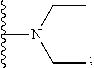 [SMILES: *N(CC)CC]

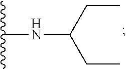 [SMILES: *NC(CC)CC]

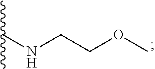 [SMILES: *NCCOC]

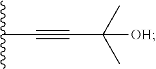 [SMILES: *C#CC(C)(C)O]

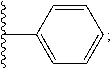 [SMILES: *c1ccccc1]

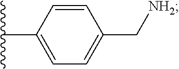 [SMILES: *c1ccc(CN)cc1]

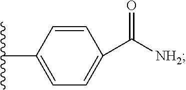 [SMILES: *c1ccc(C(N)=O)cc1]

or

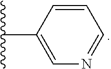 [SMILES: *c1cccnc1]

In another embodiment of the first aspect of the invention, R⁷ is halo.

In another embodiment of the first aspect of the invention, R⁷ is —F.

In another embodiment of the first aspect of the invention, R⁷ is

| | | |
|---|---|---|
| —H; | —F; | or —CH₃. |

In another embodiment of the first aspect of the invention, R⁹ is —OH.

In another embodiment of the first aspect of the invention, R⁹ is

—H;
—OH;
—CH₃;
—CF₃;
—CH₂CH₃;
—CO₂H;
—CO₂CH₃;

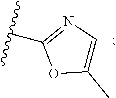 [SMILES: *c1ncc(C)o1]

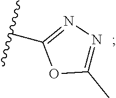 [SMILES: *c1nnc(C)o1]

or

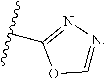 [SMILES: *c1nnco1]

In another embodiment of the first aspect of the invention, R¹⁰ is —H or —C₁-C₆ alkyl.

In another embodiment of the first aspect of the invention, R¹⁰ is —H.

In another embodiment of the first aspect of the invention, R¹⁰ is —C₁-C₆ alkyl.

In another embodiment of the first aspect of the invention, R¹⁰ is —CH₃.

In another embodiment of the first aspect of the invention, R¹¹ is —H or —C₁-C₆ alkyl.

In another embodiment of the first aspect of the invention, R¹¹ is —H.

In another embodiment of the first aspect of the invention, R¹¹ is —C₁-C₆ alkyl.

In another embodiment of the first aspect of the invention, R¹¹ is —CH₃.

In another embodiment of the first aspect of the invention, R¹⁰ and R¹¹ are independently

| | |
|---|---|
| —H; | or —CH₃. |

In another embodiment of the first aspect of the invention, R¹² is —H or —C₁-C₆ alkyl.

In another embodiment of the first aspect of the invention, R¹² is —H.

In another embodiment of the first aspect of the invention, R¹² is —C₁-C₆ alkyl.

In another embodiment of the first aspect of the invention, R¹² is —CH₃.

In another embodiment of the first aspect of the invention, R¹³ is —H or —C₁-C₆ alkyl.

In another embodiment of the first aspect of the invention, R¹³ is —H.

In another embodiment of the first aspect of the invention, R¹³ is —C₁-C₆ alkyl.

In another embodiment of the first aspect of the invention, R¹³ is —CH₃.

In another embodiment of the first aspect of the invention, R¹² and R¹³ are independently

| | |
|---|---|
| —H; | or —CH₃. |

In another embodiment of the first aspect of the invention, R¹⁴ is —H, —C₁-C₆ alkyl.

In another embodiment of the first aspect of the invention, R¹⁴ is —H.

In another embodiment of the first aspect of the invention, R¹⁴ is —C₁-C₆ alkyl.

In another embodiment of the first aspect of the invention, R¹⁴ is —CH₃.

In another embodiment of the first aspect of the invention, R¹⁴ is

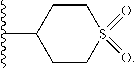 [SMILES: *C1CCS(=O)(=O)CC1]

In another embodiment of the first aspect of the invention, R¹⁴ is

—H;
—CH₃;
—C(O)H;
—C(O)CH₃;

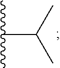 [SMILES: *C(C)C]

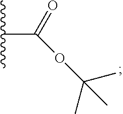 [SMILES: *C(=O)OC(C)(C)C]

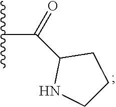 [SMILES: *C(=O)C1CCCN1]

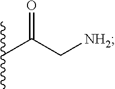 [SMILES: *C(=O)CN]

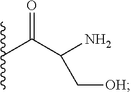 [SMILES: *C(=O)C(N)CO]

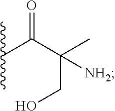 [SMILES: *C(=O)C(C)(N)CO]

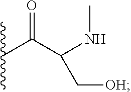 [SMILES: *C(=O)C(CO)NC]

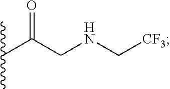 [SMILES: *C(=O)CNCC(F)(F)F]

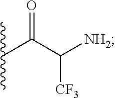 [SMILES: *C(=O)C(N)C(F)(F)F]

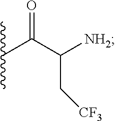 [SMILES: *C(=O)C(N)CC(F)(F)F]

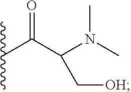 [SMILES: *C(=O)C(CO)N(C)C]

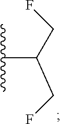 [SMILES: *C(CF)CF]

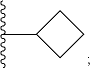 [SMILES: *C1CCC1]

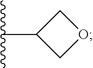 [SMILES: *C1COC1]

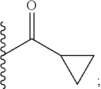 [SMILES: *C(=O)C1CC1]

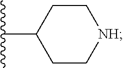 [SMILES: *C1CCNCC1]

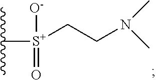 [SMILES: *[S+](=O)([O-])CCN(C)C]

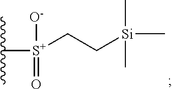 [SMILES: *[S+](=O)([O-])CC[Si](C)(C)C]

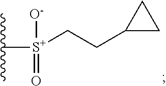 [SMILES: *[S+](=O)([O-])CCC1CC1]

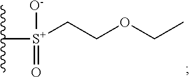 [SMILES: *[S+](=O)([O-])CCOCC]

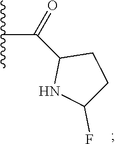 [SMILES: *C(=O)C1CCC(F)N1]

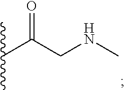 [SMILES: *C(=O)CNC]

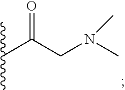 [SMILES: *C(=O)CN(C)C]

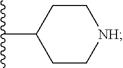 [SMILES: *C1CCNCC1]

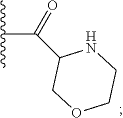 [SMILES: *C(=O)C1COCCN1]

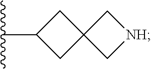 [SMILES: *C1CC2(CNC2)C1]

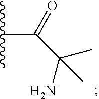 [SMILES: *C(=O)C(C)(C)N]

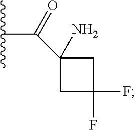 [SMILES: *C(=O)C1(N)CC(F)(F)C1]

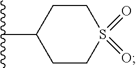 [SMILES: *C1CCS(=O)(=O)CC1]

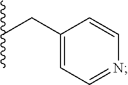 [SMILES: *Cc1ccncc1]

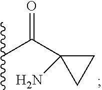 [SMILES: *C(=O)C1(N)CC1]

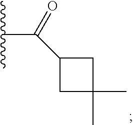 [SMILES: *C(=O)C1CC(C)(C)C1]

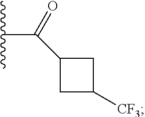 [SMILES: *C(=O)C1CC(C(F)(F)F)C1]

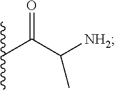 [SMILES: *C(=O)C(C)N]

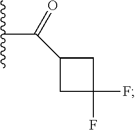 [SMILES: *C(=O)C1CC(F)(F)C1]

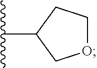 [SMILES: *C1CCOC1]

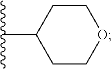 [SMILES: *C1CCOCC1]

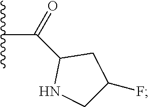 [SMILES: *C(=O)C1CC(F)CN1]

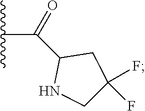 [SMILES: *C(=O)C1CC(F)(F)CN1]

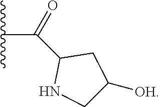 [SMILES: *C(=O)C1CC(O)CN1]

In a second aspect, the disclosure provides a compound of Formula II.

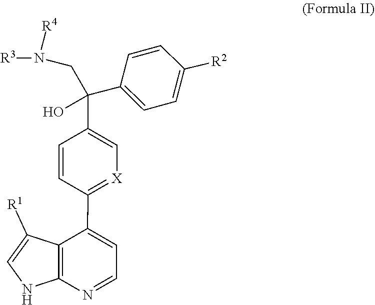

or a pharmaceutically acceptable salt thereof,
wherein
X is C or N;
R¹ is —H, halo, a 6-10 membered aryl ring, a 5-10 membered heteroaryl ring containing 1 or 2 heteroatoms, or —C(O)O—C₁-C₆ alkyl;
R² is —H or halo;
R³ is —H or —C₁-C₆ alkyl; and
R⁴ is —H, —C(O)—R^{a}, or

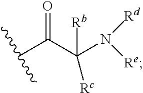

R^{a} is a 4-6 membered heteroaryl ring containing 1 or 2 heteroatoms, or —C₁-C₆ alkyl, wherein the —C₁-C₆ alkyl is optionally substituted with one or more of —OH, —P(O)(OH)₂;
R^{b} and R^{c} are independently selected from —H and —C₁-C₆ alkyl;
R^{d} is —H or —C₁-C₆ alkyl; and
R^{e} is —H, —C₁-C₆ alkyl, or —C(O)—CH₂NH₂.

In one embodiment of the second aspect of the invention, the compound is a compound of Formula IIA

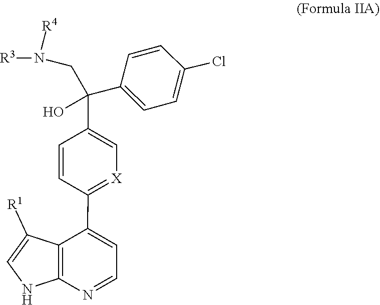

or a pharmaceutically acceptable salt thereof, wherein the R groups are as defined for Formula II.

In another embodiment of the second aspect of the invention, the compound is a compound of Formula IIB

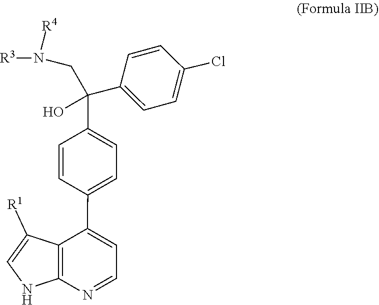

or a pharmaceutically acceptable salt thereof, wherein the R groups are as defined for Formula II.

In another embodiment of the second aspect of the invention, the compound is a compound of Formula IIC

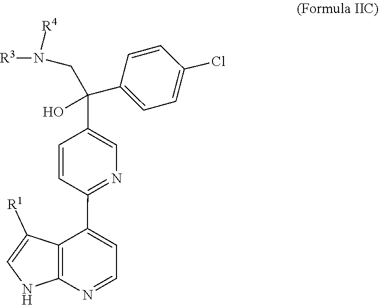

or a pharmaceutically acceptable salt thereof, wherein the R groups are as defined for Formula II.

In another embodiment of the second aspect of the invention, R¹ is H or halo.

In another embodiment of the second aspect of the invention, R¹ is H.

In another embodiment of the second aspect of the invention, R¹ is halo.

In another embodiment of the second aspect of the invention, R¹ is F or Cl.

In another embodiment of the second aspect of the invention, R³ is —H.

In another embodiment of the second aspect of the invention, R⁴ is —H.

In another embodiment of the second aspect of the invention, R⁴ is —C(O)—R^{a}.

In another embodiment of the second aspect of the invention, R⁴ is

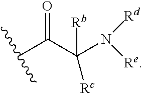

The compounds of the invention may be synthesized in accordance with the synthetic protocols set forth in the Examples, infra.

The present invention encompasses all suitable combinations of the various embodiments of each aspect of the invention as disclosed above. Thus, it is to be understood that all combinations of embodiments resulting in a stable compound are to be regarded as disclosed herein, even if not specifically recited. Combinations of substituents and/or variables are permissible only if such combinations result in stable compounds. A stable compound or stable structure is meant to imply a compound that is sufficiently robust to survive isolation from a reaction mixture, and subsequent formulation into an effective therapeutic agent. It is within the purview of one of skill in the art to envision various sub-genera and stable compounds based on the disclosure herein, and accordingly each possible sub-genus and compound is to be considered specifically disclosed herein.

Certain compounds are described herein using a general formula that includes variables, e.g. R^{a}, R^{b}, R¹, and R². Unless otherwise specified, each variable within such a formula is defined independently of other variables. Thus, if a group is said to be substituted, e.g. with 0-2 R*, then said group may be substituted with up to two R* groups and R* at each occurrence is selected independently from the definition of R*.

When ranges are specified with respect to particular moieties, it is to be understood that all options and sub-ranges within the range are included, even if not specifically enumerated. For example, C₁-C₆ alkyl includes C₁ alkyl, C₂ alkyl, C₃ alkyl, C₄ alkyl, C₅ alkyl, and C₆ alkyl, as well as C₂-C₆ alkyl, C₂-C₄ alkyl, etc.

The compounds of the present disclosure may have asymmetric centers. Compounds of the present disclosure containing an asymmetrically substituted atom may be isolated in optically active or racemic forms. It is known in the art how to prepare optically active forms, such as by resolution of materials. All chiral, diastereomeric, all mixtures of chiral or diasteromeric forms, and racemic forms are within the scope of this disclosure, unless the specific stereochemistry or isomeric form is specifically indicated. Where stereochemistry is not specified, it is to be understood that the compounds of the present disclosure include all possible stereoisomers, individually or as mixtures in any ratio. It will also be understood by a person of ordinary skill in the art that when a compound is denoted as (R) or (S) stereoisomer, it may contain the corresponding (S) or (R) stereoisomer as an impurity preferably the undesired enantiomer is present in less than about 10%, preferably 5% w/w. About means + or −10% of initial value.

Certain compounds of the present disclosure can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group even if not specifically recited herein. Similarly, when the cyclic groups such as aryl, heteroaryl, heterocyclyl are substituted, they include all the positional isomers even if not specifically recited herein. Furthermore, all hydrates of a compound of the present disclosure are within the scope of this disclosure.

The present disclosure also includes protected derivatives of compounds of the disclosure. For example, when compounds of the present disclosure contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of the present disclosure can be prepared by methods well known in the art.

The disclosure of the compounds of Formula I and Formula II includes all isotopes of atoms occurring in the present compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C and isotopes of fluorine including ¹⁹F.

The compounds of Formula I, and pharmaceutical compositions thereof, are useful as inhibitors of LATS1 and/or LATS2 (“LATS1/2”), i.e. they inhibit one or both of LATS1 and LATS2. In certain embodiments, the compounds of Formula I are selective inhibitors of LATS1/2 relative to AKT, i.e. they exhibit greater inhibition of LATS1/2 than of AKT. In certain embodiments, the selective LATS1/2 inhibitors have an inhibitory capacity for LATS 1 and/or LATS2 that is at least about 10-fold greater than their inhibitory capacity for AKT. In certain embodiments, the selective LATS1/2 inhibitors have an inhibitory capacity for LATS 1 and/or LATS2 that is at least about 100-fold, at least about 200-fold, at least about 500-fold, at least about 1000-fold, at least about 2000-fold, or at least about 5000-fold greater than their inhibitory capacity for AKT, or any range formed by the recited values, e.g. about 100-fold to about 500-fold, about 10-fold to about 1000-fold, etc. The compounds of Formula II, and pharmaceutical compositions thereof, are useful as dual LATS1/2 and AKT inhibitors.

### Pharmaceutical Compositions and Administration

Compounds disclosed herein can be administered as the neat chemical, but are preferably administered as a pharmaceutical composition. Accordingly, in further aspects, the present invention is directed to compositions comprising the compounds of the invention and a pharmaceutically acceptable carrier, excipient, or diluent. Use of the phrase “pharmaceutically acceptable carrier, excipient, or diluent” is intended to encompass a single or a mixture of pharmaceutically acceptable ingredients.

Acceptable carriers, excipients, and diluents are non-toxic, aid administration, and do not adversely affect the therapeutic benefit of the compound of this disclosure. They must be of sufficiently high purity and sufficiently low toxicity to render them suitable for administration to the subject being treated. They may be inert or they may possess pharmaceutical benefits of their own. They may be any solid, liquid, semi-solid or, in the case of an aerosol composition, gaseous excipient. The amount of carrier, excipient or diluent employed in conjunction with the compound is sufficient to provide a practical quantity of material for administration per unit dose of the compound.

Suitable carriers, excipients, and diluents are known to those of skill in the art. They may include, but are not limited to, solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like; and liquid and semisolid excipients such as glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this disclosure in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 20th ed., 2000).

Optional active agents may be included in a pharmaceutical composition, which do not substantially interfere with the activity of the compound of the present invention.

The compounds of this disclosure may be used in combination with one or more other drugs in the treatment of diseases or conditions for which compounds of this disclosure or the other drugs may have utility. Such other drug(s) may be administered by a route and in an amount commonly used therefor, contemporaneously or sequentially with a compound of the present disclosure. When a compound of this disclosure is used contemporaneously with one or more other drugs, a pharmaceutical composition in unit dosage form containing such other drugs and the compound of the present disclosure is preferred. However, the combination therapy may also include therapies in which the compound of this disclosure and one or more other drugs are administered on different overlapping schedules. It is also contemplated that when used in combination with one or more other active ingredients, the compounds of the present disclosure and the other active ingredients may be used in lower doses than when each is used singly.

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients. For example, the compound is present at a level of about 1-80 wt. %.

Compounds disclosed herein may be administered orally, topically, parenterally, by inhalation or spray, sublingually, transdermally, via buccal administration, or by other means routine in the art for administering pharmaceutical compositions. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

In general, the compounds of this disclosure will be administered in a therapeutically effective amount by any of the accepted modes of administration for agents that serve similar utilities. Therapeutically effective amounts of compounds of this disclosure may range from about 0.01 to about 500 mg per kg patient body weight per day, which can be administered in single or multiple doses. A suitable dosage level may be from about 0.1 to about 250 mg/kg per day; or about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the patient, the potency of the compound being utilized, the route and form of administration, and other factors.

### Methods

The compounds of Formula I as disclosed herein are useful as LATS1/2 inhibitors. Accordingly, one aspect of the invention provides for a method of inhibiting one or more of large tumor suppressor kinase 1 (LATS1) and large tumor suppressor kinase 2 (LATS2) in a subject in need thereof, comprising administering to the subject a compound of Formula I, or a pharmaceutical composition comprising a compound of Formula I.

The disclosure also provides a method of treating, preventing, or ameliorating a disease, disorder, or condition associated with LATS1 or LATS2 in a subject in need thereof comprising administering to the subject a therapeutically effective amount of a compound of Formula I, or a pharmaceutical composition comprising a compound of Formula I.

The disease, disorder, or condition associated with LATS1 or LATS2 includes, but is not limited to, a wound, a disease that would benefit from organ or cellular regeneration, cancer, and heavy metal poisoning.

The wound may be an acute wound or it may be a chronic wound. Acute wounds include, but are not limited to, a surgical incision, a bite, a cut, a laceration (which may include, e.g., a scratch), a burn, a gunshot wound, an abrasion, and a shrapnel wound. Chronic wounds include, but are not limited to a foot ulcer, a leg ulcer, a wound associated with vascular disease, and a wound associated with diabetes.

A disease that would benefit from organ or cellular regeneration includes, but is not limited to, a disease of the liver, a disease of the kidney, a disease of the heart, a disease of the lung, and a disease of other organs. The disease of the liver may be, but is not limited to, non-alcoholic steatohepatitis (NASH), cholestatic liver injury, alcohol-related liver disease, cirrhosis, non-alcoholic fatty liver disease (NAFLD), and hepatitis. The disease of the kidney may be, but is not limited to, chronic kidney disease. The disease of the heart may be, but is not limited to, coronary artery disease and heart failure. The disease of the lung may be, but is not limited to, chronic obstructive pulmonary disease (COPD), complications from COVID, emphysema, pneumoconiosis, and acute respiratory distress syndrome. The disease of other organs may be, but is not limited to, spinal disease or injury, nerve disease or injury, hearing loss, loss of sight, and alopecia.

The cancer may be, but is not limited to, colon cancer or breast cancer.

The heavy metal poisoning may be, but is not limited to, cadmium poisoning, lead poisoning, arsenic poisoning, zinc poisoning, and mercury poisoning.

Additionally, the compounds of Formula I may be used in a method of promoting ex vivo growth of a cell line or of a cell product comprising exposing the cell line or cell product to a compound of Formula I, or to a composition or pharmaceutical composition comprising a compound of Formula I.

The cell line or cell product may be a therapeutic product, i.e. a product use to treat a disease, disorder, or condition in a subject in need of such treatment. In certain embodiments, the therapeutic product may be for the treatment of human diseases.

The cell line or cell product may also be stem cells or a cell product derived from stem cells. In certain embodiments, the cell line or the cell product is induced pluripotent stem cells (IPSCs) or a cell product derived from IPSCs.

In certain embodiments, the cell line or cell product may be a cell line is a human cell line used for research purposes. In other embodiments, the cell line or cell product may be used to express biological products.

The present disclosure also provides for a cell population or a cell product obtained by the ex vivo methods disclosed herein.

The present invention also provides for a method of accelerating tissue growth ex vivo comprising exposing the tissue to a compound of Formula I, or to a composition or pharmaceutical composition comprising a compound of Formula I.

The compounds of Formula II as disclosed herein are useful as dual LATS1/2 and AKT inhibitors. Accordingly, a further aspect of the invention provides for a method of jointly inhibiting protein kinase B (AKT) and one or more of large tumor suppressor kinase 1 (LATS1) and large tumor suppressor kinase 2 (LATS2) in a subject in need thereof, comprising administering to the subject a compound of Formula II, or a pharmaceutical composition comprising a compound of Formula II.

The disclosure further provides a method of treating, preventing, or ameliorating a disease, disorder, or condition associated with LATS1, LATS2, or AKT in a subject in need thereof comprising administering to the subject a therapeutically effective amount of a compound of Formula II, or a pharmaceutical composition comprising a compound of Formula II.

The disease, disorder, or condition or condition associated with LATS1, LATS2, or AKT is as discussed above with respect to the administration of compounds of Formula I. In a further embodiment of the methods of the invention with respect to Formula II, the cancer may be a cancer having PI3K/AKT hyperactivity, including, but not limited to, a HR+, HER2-amplified, or triple negative breast cancer.

Additionally, the compounds of Formula II may be used in a method of promoting ex vivo growth of a cell line or of a cell product comprising exposing the cell line or cell product to a compound of Formula II, or to a composition or pharmaceutical composition comprising a compound of Formula II. The cell line or cell product may be as described above in relation to Formula I.

The present disclosure also provides for a cell population or a cell product obtained by the ex vivo methods disclosed herein with respect to Formula II.

The present invention also provides for a method of accelerating tissue growth ex vivo comprising exposing the tissue to a compound of Formula II, or to a composition or pharmaceutical composition comprising a compound of Formula II.

Both human and non-human subjects are within the scope of the disclosure. Non-human subjects include, but are not limited to, fish, amphibians, reptiles or birds, but a particular embodiment of the disclosure includes treating mammals, including non-human mammals such as rodents (rats, guinea pigs), companion animals (e.g. cats, dogs), or livestock animals (sheep, goats, pigs, cattle, horses). In certain embodiments, the subject is a mammal, and in other embodiments, the subject is a human.

Unless otherwise specified, reference to Formula I with respect to the methods disclosed herein refers to any compound or combination of compounds of Formula I, including compounds of Formulas IA, IB, IC, ID, and IE. Reference to Formula II with respect to the methods disclosed herein refers to any compound or combination of compounds of Formula II, including compounds of Formulas IIA, IIB, and IIC.

The following examples are provided to enable those skilled in the art to more clearly understand and practice the present disclosure. They should not be considered as limiting the scope of the disclosure, but merely as being illustrative and representative thereof. The compounds of the present invention may be synthesized using the methods described below, together with synthetic methods known in the art of synthetic organic chemistry, or by variations thereon. Those skilled in the art will understand that the synthetic routes disclosed herein may be modified (e.g. alternative starting materials, reagents, reaction conditions, etc.), or that other synthetic routes may be utilized to synthesize the compounds of the invention. Such alternatives are within the purview of one of skill in the art.

### EXAMPLES

### Example 1: LATS Inhibitors: General Methods

All commercially available reagents and solvents were purchased and used without further purification. All microwave reactions were carried out in a sealed microwave vial equipped with a magnetic stir bar and heated in a Biotage Initiator Microwave Synthesizer. ¹H NMR spectra were recorded on Varian 400 MHz spectrometers in CD₃OD, CD3CN, CDCl3, or D6-DMSO as indicated. For spectra recorded in CD₃OD, chemical shifts are reported in ppm with CD₃OD (3.31 ppm) as reference for ¹H NMR spectra. For spectra recorded in CDCl3, chemical shifts are reported in ppm relative to deuterochloroform (7.26 ppm for ¹H NMR). For spectra recorded in CD3CN, chemical shifts are reported in ppm relative to CD3CN (1.93 ppm for ¹H NMR). For spectra in D6-DMSO chemical shifts are reported in ppm relative to D6-DMSO (2.50 ppm for ¹H NMR). The coupling constants (J value) are reported as Hertz (Hz). The splitting patterns of the peaks were described as: singlet (s); doublet (d); triplet (t); quartet (q); multiplet (m) and septet (septet).

Compounds were analyzed on an Agilent 1200 series LC/MS equipped with a Luna C18 (3 mm×75 mm, 3 μm) reversed-phase column with UV detection at λ=220 nm and λ=254 nm. The mobile phase consisted of water containing 0.05% trifluoroacetic acid as component A and acetonitrile containing 0.025% trifluoroacetic acid as component B. A linear gradient was run as follows: 0 min 4% B; 7 min 100% B; 8 min 100% B at a flow rate of 0.8 ml/min.

Reverse phase chromatography for purification purposes was performed on a Waters semi-preparative HPLC equipment. The column used was a Phenomenex Luna C18 (5 μm, 30×75 mm) at a flow rate of 45 mL/min. The mobile phase consisted of acetonitrile and water (each containing 0.1% trifluoroacetic acid, 0.1% formic acid, or 0.1% ammonium hydroxide, as indicated). A gradient of 5%-100% acetonitrile in water was used during the purification. Fraction collection was triggered by UV detection (220 nm).

### Example 2: LATS Inhibitors: General Synthetic Protocols and Schemes

Certain compounds of the invention were synthesized by the following general synthetic protocols

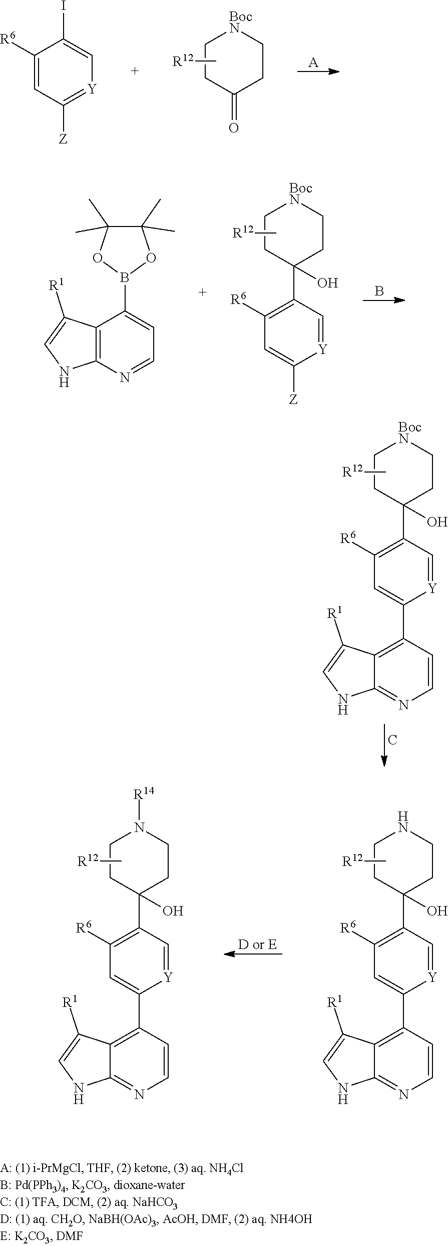

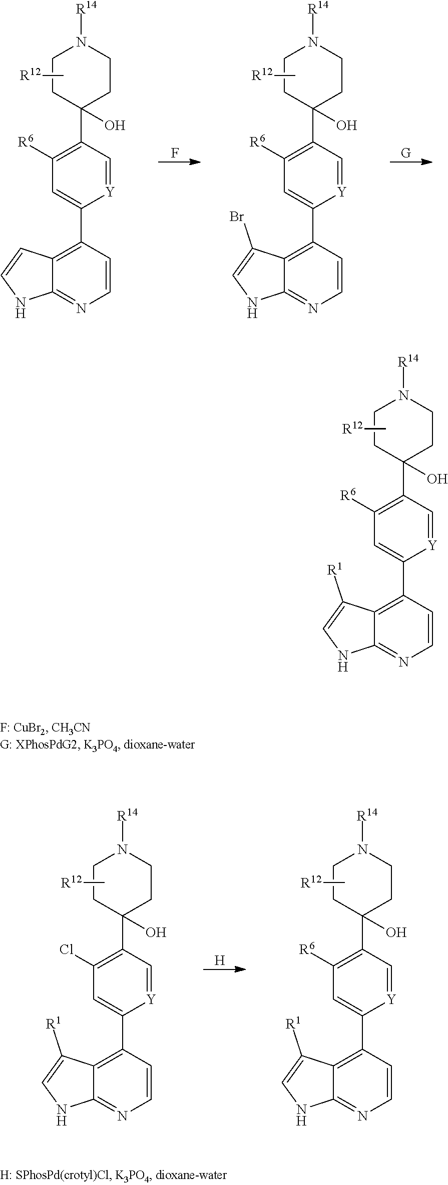

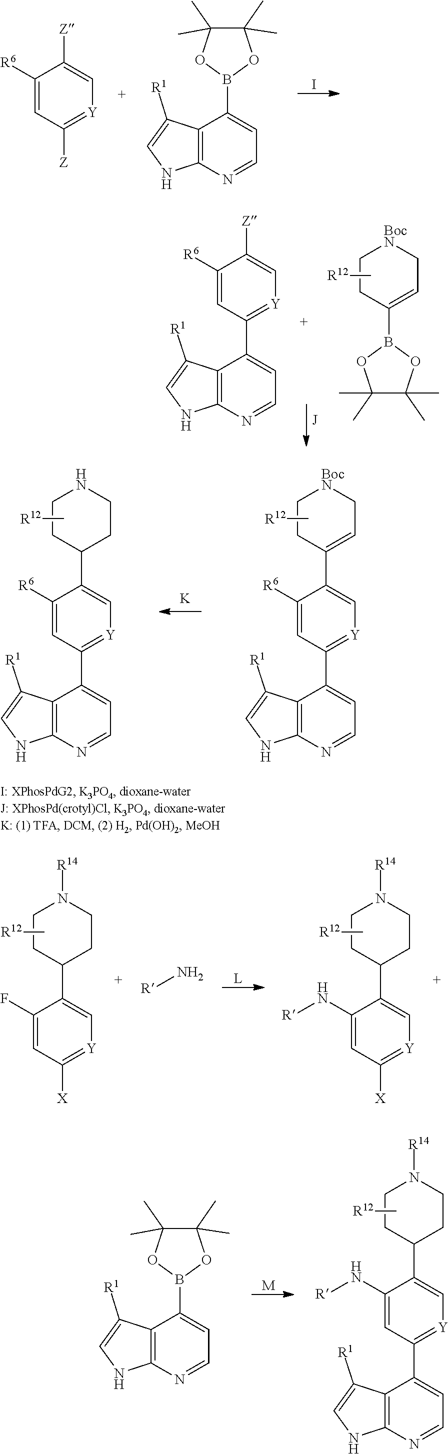

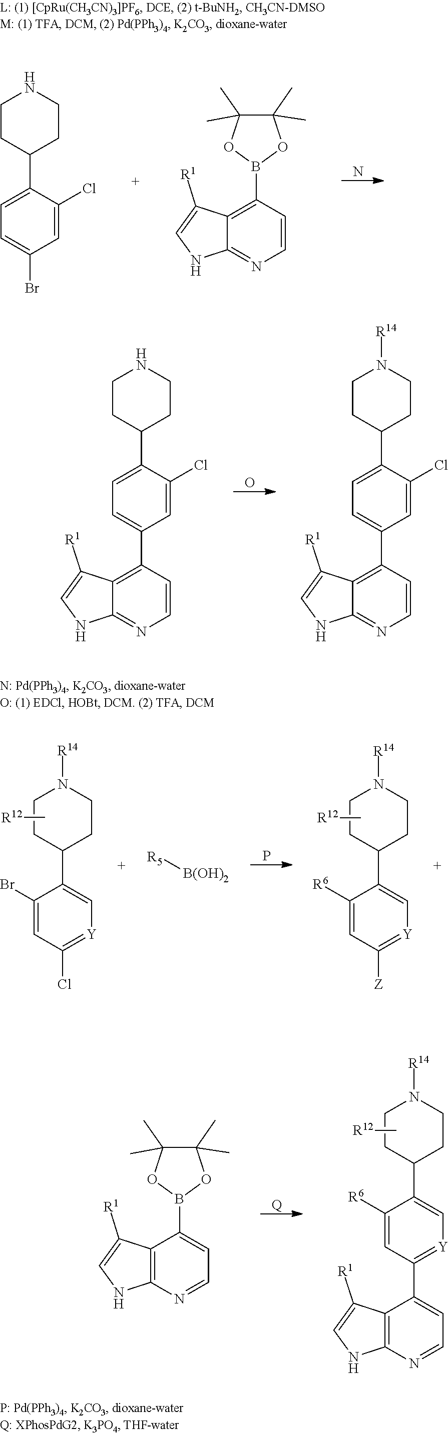

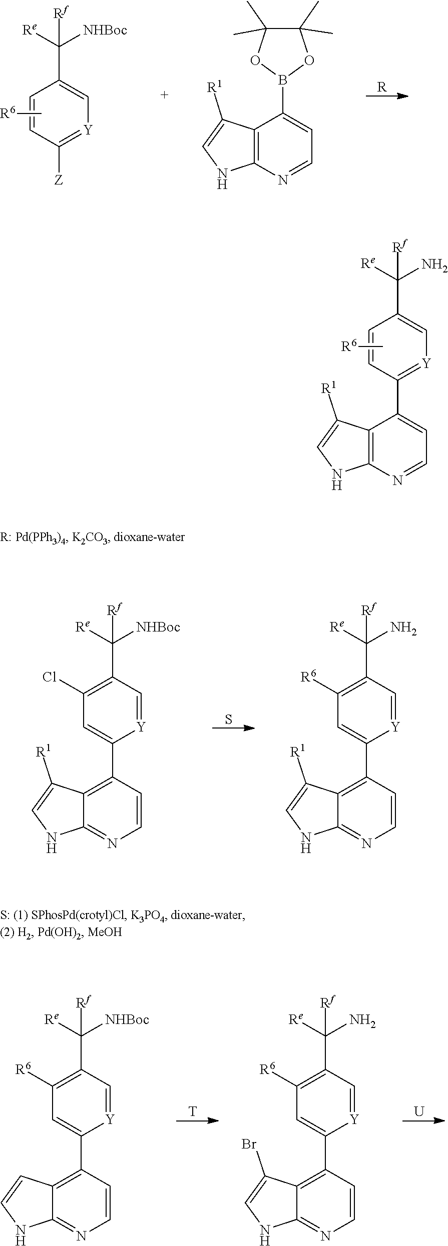

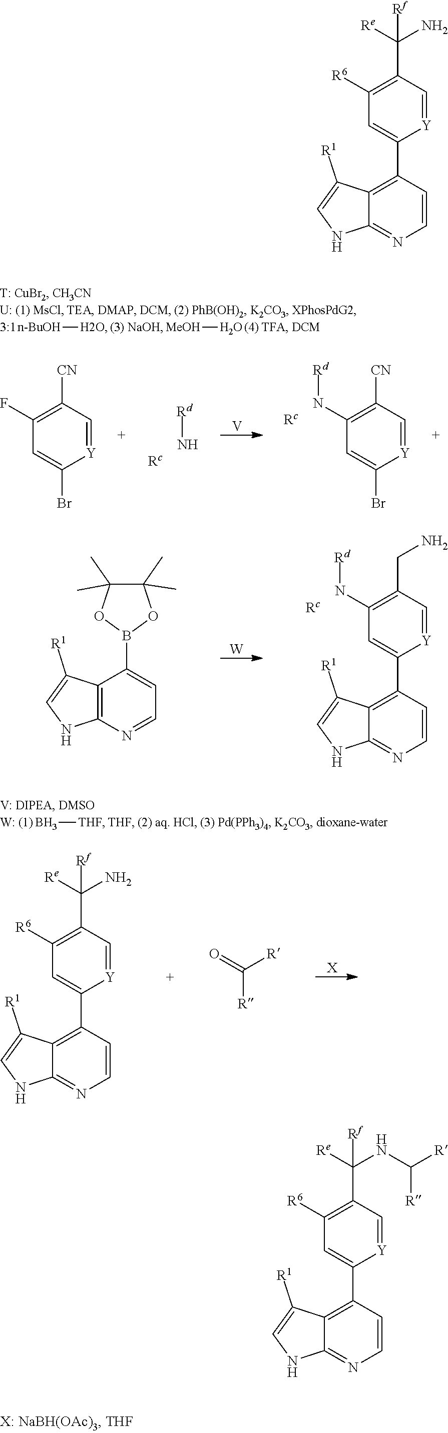

### Example 3: LATS Inhibitors: Synthesis

### Synthesis of Compounds of General Formula A1 and A2

Compounds of general formula A1 and A2 can be prepared as illustrated in Scheme 1 below. Intermediate 1b can also be prepared as per alternate procedure Scheme 1a.

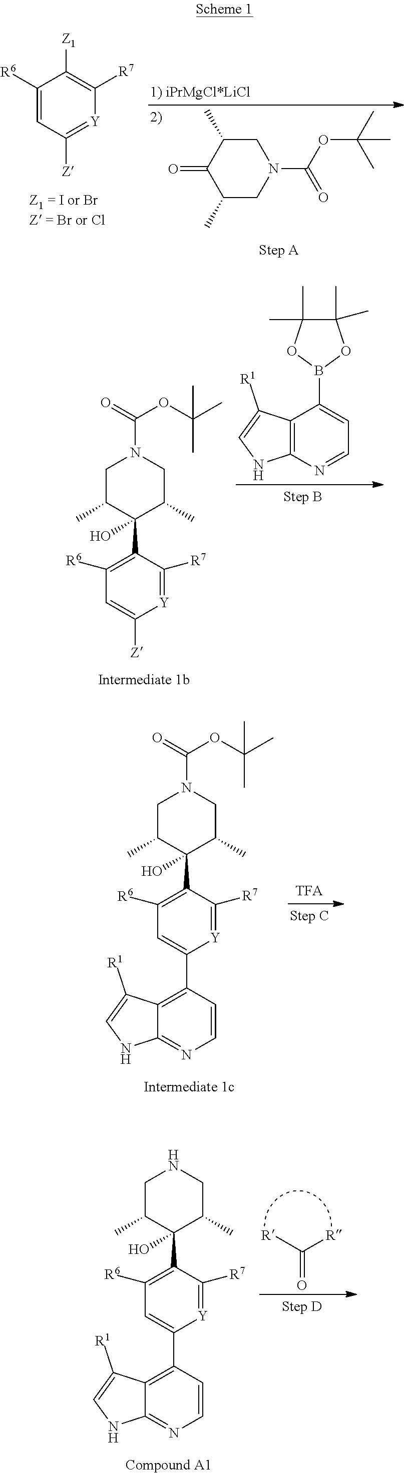

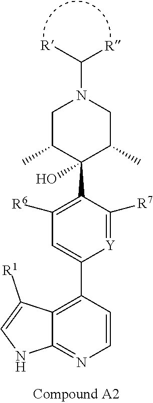

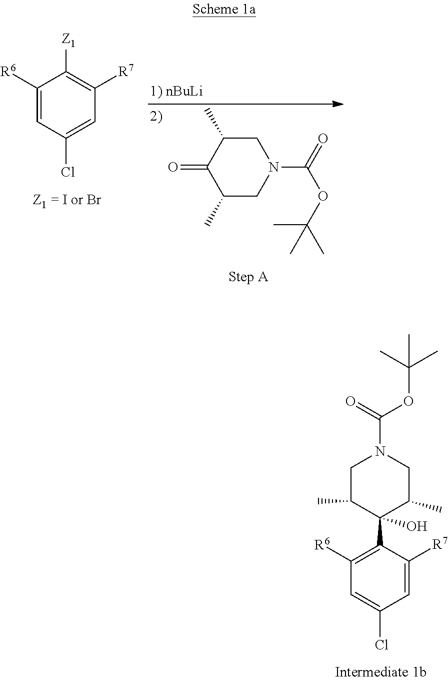

### Synthesis of Intermediates and Compounds of Scheme 1

### Intermediate 1b-1 (Scheme 1): tert-butyl (3S,5R)-4-(4-bromo-2-chlorophenyl)-4-hydroxy-3,5-dimethylpiperidine-1-carboxylate

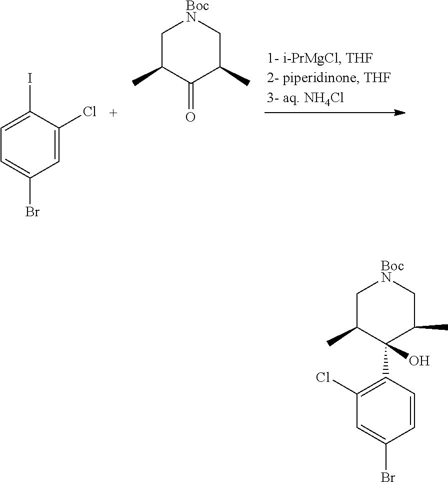

Step A: An oven-dried flask was charged with 4-bromo-2-chloro-1-iodobenzene (2.33 g, 7.33 mmol) and tetrahydrofuran (11.7 mL). The resulting solution was cooled down to 0-5° C. using an ice-water bath. Then, an iso-propyl magnesium chloride lithium chloride complex solution (1.3 M in tetrahydrofuran) (7.30 mL, 9.53 mmol) was added drop wise. The cooling bath was removed, and the resulting solution was stirred at 23° C. for 1.0 h. Then, a solution containing tert-butyl (3R,5S)-3,5-dimethyl-4-oxopiperidine-1-carboxylate (2.00 g, 8.80 mmol) in tetrahydrofuran (2.90 mL) was added slowly, and the resulting reaction mixture was allowed to warm up progressively to room temperature and was stirred at reflux for 1.5 h. The reaction mixture was cooled by the mean of an ice-water bath, and the reaction was quenched upon addition of 5.0 mL of saturated aqueous NH₄Cl. The reaction mixture was partitioned between 20 mL of water and 20 mL of tert-butyl methyl ether. The layers were separated; the aqueous layer was extracted with two 20-mL portions of tert-butyl methyl ether. The combined organic layer was washed with 20 mL of brine, was then dried (sodium sulfate), and was then concentrated under diminished pressure. The residue was applied to a silica gel column; eluting with 100:0→50:50 hexanes-ethyl acetate provided the title compound; ¹H NMR (400 MHz, DMSO-d₆) δ 7.74 (d, J=8.6 Hz, 1H), 7.63 (d, J=1.8 Hz, 1H), 7.57 (dd, J=8.6, 2.1 Hz, 1H), 4.99 (s, 1H), 3.70 (s, 2H), 2.75 (dq, J=11.8, 5.6 Hz, 2H), 1.41 (d, J=0.9 Hz, 9H), 0.97 (d, J=6.8 Hz, 2H), 0.48 (d, J=6.2 Hz, 6H). LC-MS (m/z [M−C(CH₃)₃+2H]⁺): 364.0

### Intermediates 1b-2 to 1b-15 (Scheme 1)

Intermediates 1b-2 to 1b-15 were prepared using an analogous procedure to that of Intermediate 1b-1, and are shown in Table 1.

TABLE 1

| Compound | Structure | IUPAC name | LCMS m/z |
|---|---|---|---|
| 1b-2 | 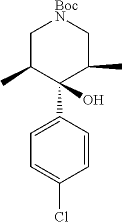 | tert-butyl (3S,4s,5R)-4-(4- chlorophenyl)-4-hydroxy-3,5- dimethylpiperidine-1-carboxylate | [M − C(CH₃)₃ + 2H]⁺: 284.1 |
| 1b-3 | 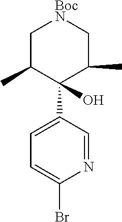 | tert-butyl (3S,4s,5R)-4-(6- bromopyridin-3-yl)-4-hydroxy- 3,5-dimethylpiperidine-1- carboxylate | [M + H]⁺: 385.0 |
| 1b-4 | 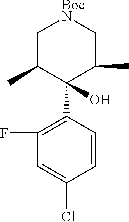 [SMILES: C[C@@H]1CN(C(=O)OC(C)(C)C)C[C@H](C)[C@@]1(O)c1ccc(Cl)cc1F] | tert-butyl (3S,4s,5R)-4-(4-chloro- 2-fluorophenyl)-4-hydroxy-3,5- dimethylpiperidine-1-carboxylate | [M − C(CH₃)₃ + 2H]⁺: 302.7 |
| 1b-5 | 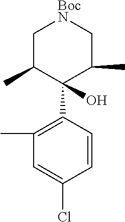 | tert-butyl (3S,4s,5R)-4-(4-chloro- 2-methylphenyl)-4-hydroxy-3,5- dimethylpiperidine-1-carboxylate | [M − C(CH₃)₃ + 2H]⁺: 298.1 |
| 1b-6 | 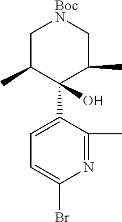 | tert-butyl (3S,4s,5R)-4-(6-bromo- 2-methylpyridin-3-yl)-4-hydroxy- 3,5-dimethylpiperidine-1- carboxylate | [M + H]⁺: 399.1 |
| 1b-7 | 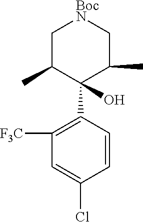 [SMILES: C[C@@H]1CN(C(=O)OC(C)(C)C)C[C@H](C)[C@@]1(O)c1ccc(Cl)cc1C(F)(F)F] | tert-butyl (3S,4s,5R)-4-(4-chloro- 2-(trifluoromethyl)phenyl)-4- hydroxy-3,5-dimethylpiperidine-1- carboxylate | [M − C(CH₃)₃ + 2H]⁺ 352.2 |
| 1b-8 | 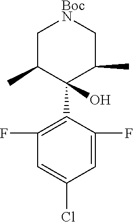 [SMILES: C[C@@H]1CN(C(=O)OC(C)(C)C)C[C@H](C)[C@@]1(O)c1c(F)cc(Cl)cc1F] | tert-butyl (3S,4s,5R)-4-(4-chloro- 2,6-difluorophenyl)-4-hydroxy- 3,5-dimethylpiperidine-1- carboxylate | [M − C(CH₃)₃ + 2H]⁺: 320.1 |
| 1b-9 | 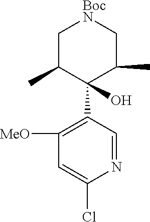 [SMILES: COc1cc(Cl)ncc1[C@@]1(O)[C@H](C)CN(C(=O)OC(C)(C)C)C[C@@H]1C] | tert-butyl (3S,4s,5R)-4-(6-chloro- 4-methoxypyridin-3-yl)-4- hydroxy-3,5-dimethylpiperidine-1- carboxylate | [M + H]⁺: 371.1 |
| 1b-10 | 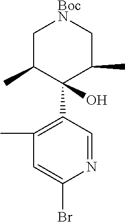 | tert-butyl (3S,4s,5R)-4-(6-bromo- 4-methylpyridin-3-yl)-4-hydroxy- 3,5-dimethylpiperidine-1- carboxylate | [M + H]⁺: 401.1 |
| 1b-11 | 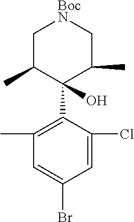 [SMILES: Cc1cc(Br)cc(Cl)c1[C@@]1(O)[C@H](C)CN(C(=O)OC(C)(C)C)C[C@@H]1C] | tert-butyl (3S,4s,5R)-4-(4-bromo- 2-chloro-6-methylphenyl)-4- hydroxy-3,5-dimethylpiperidine-1- carboxylate | [M − C(CH₃)₃ + 2H]⁺: 378.0 |
| 1b-12 | 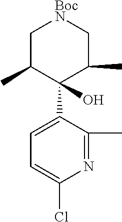 | tert-butyl (3S,4s,5R)-4-(6-chloro- 2-methylpyridin-3-y1)-4-hydroxy- 3,5-dimethylpiperidine-1- carboxylate | [M + H]⁺: 355.1 |
| 1b-13 | 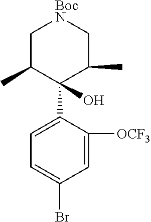 [SMILES: C[C@@H]1CN(C(=O)OC(C)(C)C)C[C@H](C)[C@@]1(O)c1ccc(Br)cc1OC(F)(F)F] | tert-butyl (3S,4s,5R)-4-(4-bromo- 2-(trifluoromethoxy)phenyl)-4- hydroxy-3,5-dimethylpiperidine-1- carboxylate | [M − C(CH₃)₃ + 2H]⁺: 412.0 |
| 1b-14 | 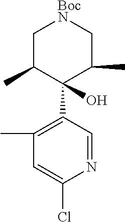 | tert-butyl (3S,4s,5R)-4-(6-chloro- 4-methylpyridin-3-yl)-4-hydroxy- 3,5-dimethylpiperidine-1- carboxylate | [M + H]⁺: 355.1 |
| 1b-15 | 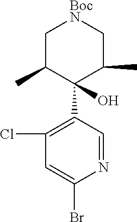 [SMILES: C[C@@H]1CN(C(=O)OC(C)(C)C)C[C@H](C)[C@@]1(O)c1cnc(Br)cc1Cl] | tert-butyl (3S,4s,5R)-4-(6-bromo- 4-chloropyridin-3-yl)-4-hydroxy- 3,5-dimethylpiperidine-1- carboxylate | [M + H]⁺: 420.9 |

### Intermediate 1b-16 (Scheme 1a): tert-butyl (3R,4s,5S)-4-(4-chloro-2-fluoro-6-methylphenyl)-4-hydroxy-3,5-dimethylpiperidine-1-carboxylate

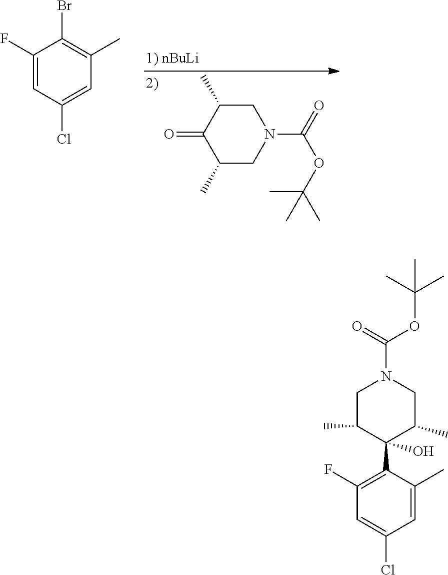

Step A: An oven-dried flask was charged with 2-bromo-5-chloro-1-fluoro-3-methylbenzene (0.450 g, 1.98 mmol) and tetrahydrofuran (4.0 mL), under a nitrogen atmosphere. The resulting solution was cooled down to −78° C. using an dry ice-acetone bath. Then n-butyl lithium (2.5 M in hexanes) (0.72 mL, 1.79 mmol) was added over 15 seconds. The reaction was allowed to proceed for 10 minutes. Then, a solution containing tert-butyl (3R,5S)-3,5-dimethyl-4-oxopiperidine-1-carboxylate (0.45 g, 1.98 mmol) in tetrahydrofuran (5.0 mL) was added slowly over approximately 30 seconds. The reaction mixture was stirred for 2 hours at −78° C. The reaction mixture was then quenched by being poured into a saturated aqeuous solution of sodium bicarbonate. The reaction mixture was extracted into ethyl acetate. The organic phase was taken and the solvent removed by rotary evaporation to give the crude product. Purification by silica gel chromatography (10% to 40% ethyl acetate in hexanes) provided the title compound. Relative stereochemistry was determined by NOE. ¹H NMR (400 MHz, Chloroform-d) δ 6.95-6.82 (m, 2H), 4.35 (bs, 1H), 3.84 (bs, 2H), 2.76 (bs, 2H), 2.59 (s, 3H), 2.57-2.43 (m, 2H), 1.47 (s, 9H), 0.70 (d, J=6.9 Hz, 6H). LC-MS (m/z [M−CO₂(CH₃)₃+2H]⁺): 272.1

### Intermediates 1b-16 to 1b-17 (Scheme 1)

Intermediates 1b-16 to 1b-17 were prepared using an analogous procedure to that of Intermediate 1b-15, and are shown in Table 2.

TABLE 2

| Compound | Structure | IUPAC name | LCMS m/z |
|---|---|---|---|
| 1b-16 | 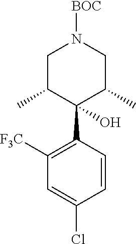 | tert-butyl (3R,4s,5S)-4-(4-chloro-2- (trifluoromethyl)-phenyl)-4-hydroxy- 3,5-dimethylpiperidine-1-carboxylate | [M − C(CH₃)₃ + 2H]⁺ 308.1 |
| 1b-17 | 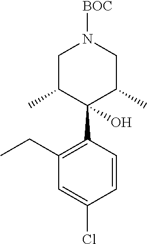 [SMILES: CCc1cc(Cl)ccc1[C@@]1(O)[C@H](C)CN(C(=O)OC(C)(C)C)C[C@@H]1C] | tert-butyl (3R,4s,5S)-4-(4-chloro-2- (ethyl)phenyl)-4-hydroxy-3,5- dimethylpiperidine-1-carboxylate | [M − C(CH₃)₃ + 2H]⁺ 268.1 |

### Intermediate 1c-1 (Scheme 1): tert-butyl (3S,5R)-4-(2-chloro-4-(3-fluoro-1H-pyrrolo[2,3-b]pyridin-4-yl)phenyl)-4-hydroxy-3,5-dimethylpiperidine-1-carboxylate

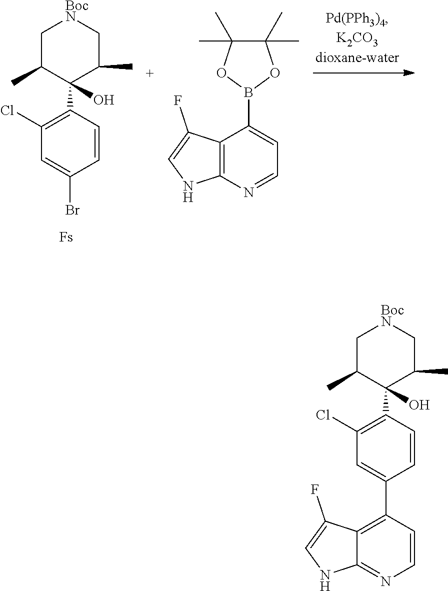

Step B: A microwave vial was charged with tert-butyl (3S,5R)-4-(4-bromo-2-chlorophenyl)-4-hydroxy-3,5-dimethylpiperidine-1-carboxylate (1.37 g, 3.27 mmol), 3-fluoro-4-(4,4,5,5-tetramethyl-1,3,2-dioxaborolan-2-yl)-1H-pyrrolo[2,3-b]pyridine (0.943 g, 3.60 mmol) and tetrakis(triphenylphosphine)palladium(0) (0.189 g, 0.164 mmol). The vial was capped and its content was purged with nitrogen twice. Then, dioxane (9.80 mL) was added, followed by 2 M aqueous potassium carbonate (2.45 mL, 4.91 mmol). The content of the reaction vial was purged with nitrogen twice, and was then heated in the microwave reactor at 120° C. for 20 min. The reaction mixture was partitioned between 20 mL of water K3 and 20 mL of ethyl acetate. The layers were separated; the aqueous layer was extracted with three 20-mL portions of ethyl acetate. The combined organic layer was washed with 20 mL of brine, was then dried (sodium sulfate), and was then concentrated under diminished pressure. The residue was applied to a silica gel column; eluting with 100:0→0:100 hexanes-ethyl acetate afforded the title compound; ¹H NMR (400 MHz, Chloroform-d) δ 9.46 (s, 1H), 8.39 (d, J=4.9 Hz, 1H), 7.88 (d, J=8.2 Hz, 1H), 7.69 (d, J=2.8 Hz, 1H), 7.60 (dd, J=8.5, 3.0 Hz, 1H), 7.21-7.14 (m, 2H), 3.96 (d, J=58.3 Hz, 2H), 3.10 (s, 2H), 2.88 (s, 2H), 1.87 (s, 1H), 1.51 (d, J=1.3 Hz, 9H), 0.65 (d, J=6.8 Hz, 6H).

### Intermediates 1c-2 to 1c-13 (Scheme 1)

Intermediates 1c-2 to 1c-13 were prepared using an analogous procedure to that of Intermediate 1c-1 and are shown in Table 3.

TABLE 3

| Compound | Structure | IUPAC name | LCMS m/z |
|---|---|---|---|
| 1c-2 | 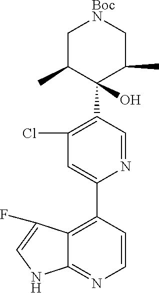 [SMILES: C[C@@H]1CN(C(=O)OC(C)(C)C)C[C@H](C)[C@@]1(O)c1cnc(-c2ccnc3[nH]cc(F)c23)cc1Cl] | tert-butyl (3R,4s,5S)-4-(4-chloro-6- (3-fluoro-1H-pyrrolo[2,3-b]pyridin- 4-yl)pyridin-3-yl)-4-hydroxy-3,5- dimethylpiperidine-1-carboxylate | [M + H]⁺: 475.2 |
| 1c-3 | 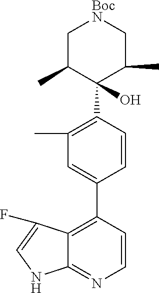 [SMILES: Cc1cc(-c2ccnc3[nH]cc(F)c23)ccc1[C@@]1(O)[C@H](C)CN(C(=O)OC(C)(C)C)C[C@@H]1C] | tert-butyl (3R,4s,5S)-4-(4-(3-fluoro- 1H-pyrrolo[2,3-b]pyridin-4-yl)-2- methylphenyl)-4-hydroxy-3,5- dimethylpiperidine-1-carboxylate | [M + H]⁺: 454.2 |
| 1c-4 | 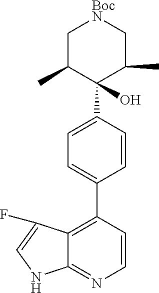 [SMILES: C[C@@H]1CN(C(=O)OC(C)(C)C)C[C@H](C)[C@@]1(O)c1ccc(-c2ccnc3[nH]cc(F)c23)cc1] | tert-butyl (3R,4s,5S)-4-(4-(3-fluoro- 1H-pyrrolo[2,3-b]pyridin-4- yl)phenyl)-4-hydroxy-3,5- dimethylpiperidine-1-carboxylate | [M + H]⁺: 440.2 |
| 1c-5 | 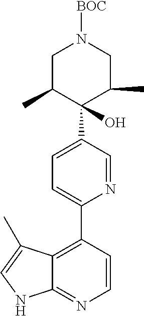 [SMILES: Cc1c[nH]c2nccc(-c3ccc([C@@]4(O)[C@H](C)CN(C(=O)OC(C)(C)C)C[C@@H]4C)cn3)c12] | tert-butyl (3R,4s,5S)-4-hydroxy-3,5- dimethyl-4-(6-(3-methyl-1H- pyrrolo[2,3-b]pyridin-4-yl)pyridin- 3-yl)piperidine-1-carboxylate | [M + H]⁺: 437.2 |
| 1c-6 | 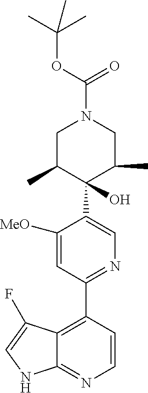 [SMILES: COc1cc(-c2ccnc3[nH]cc(F)c23)ncc1[C@@]1(O)[C@H](C)CN(C(=O)OC(C)(C)C)C[C@@H]1C] | tert-butyl (3R,4s,5S)-4-(6-(3-fluoro- 1H-pyrrolo[2,3-b]pyridin-4-yl)-4- methoxypyridin-3-yl)-4-hydroxy- 3,5-dimethylpiperidine-1- carboxylate | [M + H]⁺: 471.2 |

### Compound 1

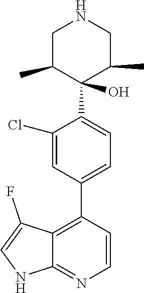 [SMILES: C[C@@H]1CNC[C@H](C)[C@@]1(O)c1ccc(-c2ccnc3[nH]cc(F)c23)cc1Cl]

### (3S,4S,5R)-4-(2-chloro-4-(3-fluoro-1H-pyrrolo[2,3-b]pyridin-4-yl)phenyl)-3,5-dimethylpiperidin-4-ol

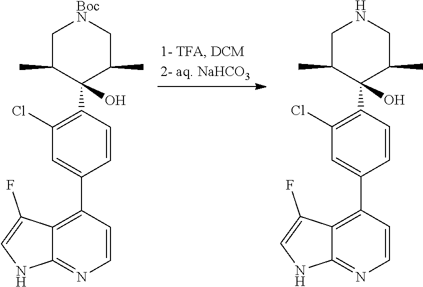 [SMILES: C[C@@H]1CN(C(=O)OC(C)(C)C)C[C@H](C)[C@@]1(O)c1ccc(-c2ccnc3[nH]cc(F)c23)cc1Cl ; C[C@@H]1CNC[C@H](C)[C@@]1(O)c1ccc(-c2ccnc3[nH]cc(F)c23)cc1Cl]

Step C: A solution containing tert-butyl (3S,5R)-4-(2-chloro-4-(3-fluoro-1H-pyrrolo[2,3-b]pyridin-4-yl)phenyl)-4-hydroxy-3,5-dimethylpiperidine-1-carboxylate (0.905 g, 1.909 mmol) in dichloromethane (19.1 mL) was treated with trifluoroacetic acid (2.20 mL, 28.6 mmol) at 23° C. for 1.5 h. The reaction mixture was concentrated under diminished pressure. The residue was dissolved into 10 mL of dichloromethane. The resulting orange solution was treated with 15 mL of saturated aqueous sodium bicarbonate. The precipitated solid was collected by filtration through a Hirsh funnel and was then dried under diminished pressure over P₂O₅ to provide the title compound; ¹H NMR (400 MHz, DMSO-d₆) δ 11.72 (s, 1H), 8.33 (d, J=4.9 Hz, 1H), 7.93 (d, J=8.2 Hz, 1H), 7.67 (dd, J=8.1, 5.3 Hz, 2H), 7.56 (d, J=1.9 Hz, 1H), 7.23 (d, J=4.9 Hz, 1H), 5.12 (s, 1H), 3.08-2.99 (m, 2H), 2.95 (dd, J=12.4, 4.0 Hz, 2H), 2.85 (t, J=12.0 Hz, 2H), 0.55 (d, J=6.7 Hz, 6H). LC-MS (m/z [M+H]⁺): 374.1

Relative stereochemistry was determined by observed NOE interactions between the alcohol peak (OH) and the methyl peaks (CH₃), the methylene peaks (CH₂) in the piperidine ring, and the methine peaks (CH) in the piperidine ring. Relative NOE intensities were calculated as OH—CH3: 100%; OH—CH2: 75.19%; OH—CH: 16.11%, indicating the alcohol and methyl groups have a respective cis configuration, with the methyl groups adopting a predominantly equatorial conformation. Other compounds were determined to have the same stereochemistry by analogy.

### Compounds 2-4

Compounds 2-4 were prepared using an analogous procedure to that of Compound 1 and are shown in Table 4.

TABLE 4

| | | | | LCMS |
|---|---|---|---|---|
| Compound | Structure | IUPAC name | ¹H NMR | m/z |
| 2 | 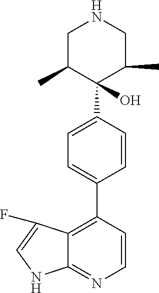 [SMILES: C[C@@H]1CNC[C@H](C)[C@@]1(O)c1ccc(-c2ccnc3[nH]cc(F)c23)cc1] | (3R,4s,5S)-4-(4-(3- fluoro-1H- pyrrolo[2,3-b]pyridin- 4-yl)phenyl)-3,5- dimethylpiperidin-4- ol | ¹H NMR (400 MHz, DMSO-d₆) δ 11.66 (s, 1H), 8.74-8.42 (m, 2H), 8.32 (d, J = 5.0 Hz, 1H), 7.70 (s, 2H), 7.53 (d, J = 2.9 Hz, 1H), 7.30 (s, 1H), 7.18 (d, J = 4.9 Hz, 1H), 5.16 (s, 1H), 3.32-3.11 (m, 2H), 2.93 (q, J = 11.7 Hz, 2H), 2.31 (dd, J = 12.1, 5.4 Hz, 2H), 0.58 (d, J = 6.7 Hz, 6H). | [M + H]⁺: 339.9 |
| 3 | 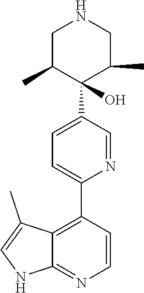 [SMILES: Cc1c[nH]c2nccc(-c3ccc([C@@]4(O)[C@H](C)CNC[C@@H]4C)cn3)c12] | (3R,4s,5S)-3,5- dimethyl-4-(6-(3- methyl-1H- pyrrolo[2,3-b]pyridin- 4-yl)pyridin-3- yl)piperidin-4-ol | | [M + H]⁺: 337.2 |
| 4 | 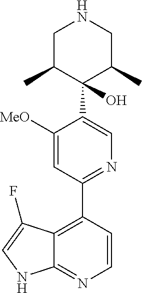 | (3R,4s,5S)-4-(6-(3- fluoro-1H- pyrrolo[2,3-b]pyridin- 4-yl)-4- methoxypyridin-3-yl)- 3,5- dimethylpiperidin-4- ol | ¹H NMR (400 MHz, DMSO-d₆) δ 11.80 (s, 1H), 8.68 (s, 1H), 8.62 (s, 2H), 8.40 (d, J = 4.9 Hz, 1H), 7.63 (s, 1H), 7.57 (s, 1H), 7.54 (d, J = 4.9 Hz, 1H), 5.37 (s, 1H), 3.99 (s, 3H), 3.09 (d, J = 11.4 Hz, 2H), 3.01-2.80 (m, 4H), 0.61 (d, J = 6.5 Hz, 6H). | [M + H]⁺: 331.2 |

### Compound 5

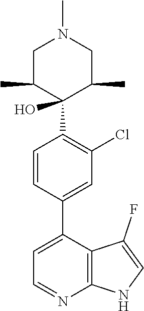 [SMILES: C[C@@H]1CN(C)C[C@H](C)[C@@]1(O)c1ccc(-c2ccnc3[nH]cc(F)c23)cc1Cl]

### (3S,4s,5R)-4-(2-chloro-4-(3-fluoro-1H-pyrrolo[2,3-b]pyridin-4-yl)phenyl)-1,3,5-trimethylpiperidin-4-ol

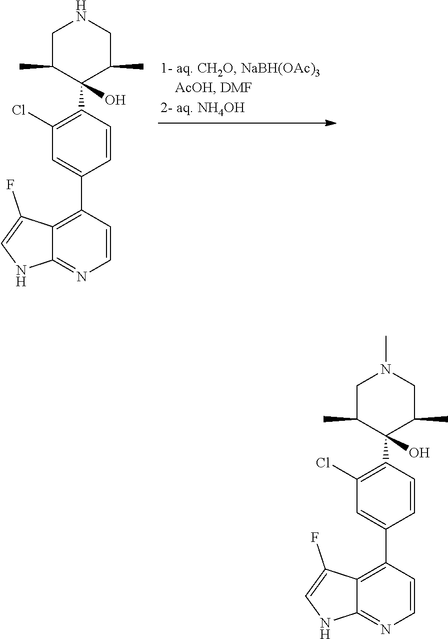 [SMILES: C[C@@H]1CNC[C@H](C)[C@@]1(O)c1ccc(-c2ccnc3[nH]cc(F)c23)cc1Cl ; C[C@@H]1CN(C)C[C@H](C)[C@@]1(O)c1ccc(-c2ccnc3[nH]cc(F)c23)cc1Cl]

Step D: (3S,5R)-4-(2-chloro-4-(3-fluoro-1H-pyrrolo[2,3-b]pyridin-4-yl)phenyl)-3,5-dimethylpiperidin-4-ol (0.200 g, 0.535 mmol) was dissolved into N,N-dimethylformamide (5.35 mL), and the resulting solution was treated with aqueous formaldehyde (0.080 mL, 1.07 mmol), acetic acid (0.046 mL, 0.802 mmol) and with sodium triacetoxyborohydride (0.170 g, 0.802 mmol) at 23° C. for 2 h. The reaction mixture was then treated 5 mL of 50% aqueous ammonium hydroxide at 23° C. for 10 min. The reaction mixture was partitioned between 10 mL of water and 10 mL of ethyl acetate; the layers were separated, then the product was extracted with three 10-mL portions of ethyl acetate. The combined organic layer was washed with 10 mL of brine, was then dried (sodium sulfate), and was then concentrated under diminished pressure. The crude product was purified by reverse-phase HPLC to provide the title compound; ¹H NMR (400 MHz, DMSO-d₆) δ 11.70 (s, 1H), 8.32 (d, J=4.9 Hz, 1H), 7.95 (d, J=8.2 Hz, 1H), 7.68-7.60 (m, 2H), 7.55 (d, J=2.5 Hz, 1H), 7.22 (d, J=4.9 Hz, 1H), 4.60 (s, 1H), 3.01 (tt, J=11.1, 6.7 Hz, 2H), 2.19 (s, 3H), 2.14 (t, J=11.2 Hz, 4H), 0.53 (d, J=6.9 Hz, 6H).

### Compound 6

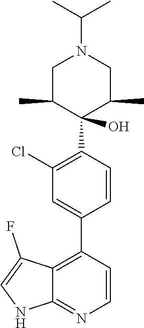 [SMILES: CC(C)N1C[C@@H](C)[C@](O)(c2ccc(-c3ccnc4[nH]cc(F)c34)cc2Cl)[C@@H](C)C1]

### (3S,4s,5R)-4-(2-chloro-4-(3-fluoro-1H-pyrrolo[2,3-b]pyridin-4-yl)phenyl)-1-isopropyl-3,5-dimethylpiperidin-4-ol

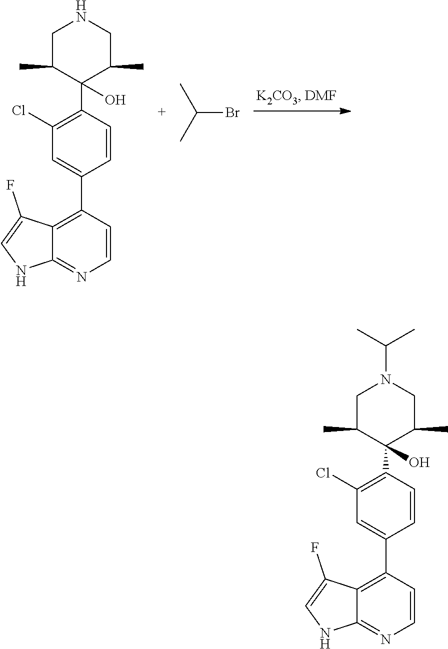

Step D: A suspension containing (3S,5R)-4-(2-chloro-4-(3-fluoro-1H-pyrrolo[2,3-b]pyridin-4-yl)phenyl)-3,5-dimethylpiperidin-4-ol (0.030 g, 0.080 mmol), 2-bromopropane (0.011 mL, 0.120 mmol) and potassium carbonate (0.022 g, 0.160 mmol) in N,N-dimethylformamide (0.800 mL) was heated in the microwave reactor at 100° C. for 20 min. The reaction mixture was diluted with 1 mL of methanol, was then filtered, and the collected solution was purified by reverse-phase HPLC to provide the title compound; ¹H NMR (400 MHz, DMSO-d₆) δ 11.71 (s, 1H), 8.33 (d, J=4.9 Hz, 1H), 8.17 (s, 1H), 7.95 (d, J=8.2 Hz, 1H), 7.65 (d, J=10.7 Hz, 2H), 7.56 (s, 1H), 7.23 (d, J=4.9 Hz, 1H), 4.81 (s, 1H), 3.17 (s, OH), 3.02 (d, J=10.8 Hz, 2H), 2.92 (s, 1H), 2.69 (d, J=11.0 Hz, 2H), 2.55 (d, J=11.2 Hz, 1H), 1.09 (d, J=6.5 Hz, 6H), 0.56 (d, J=6.9 Hz, 6H). LC-MS (m/z [M+H]⁺). 416.2

### Alternate Synthesis of Compounds of General Formula A1 and A2

Compounds of general formula A1 and A2 can also be prepared as illustrated in Scheme 2 below. Intermediates 1b are synthesized as per Scheme 1.

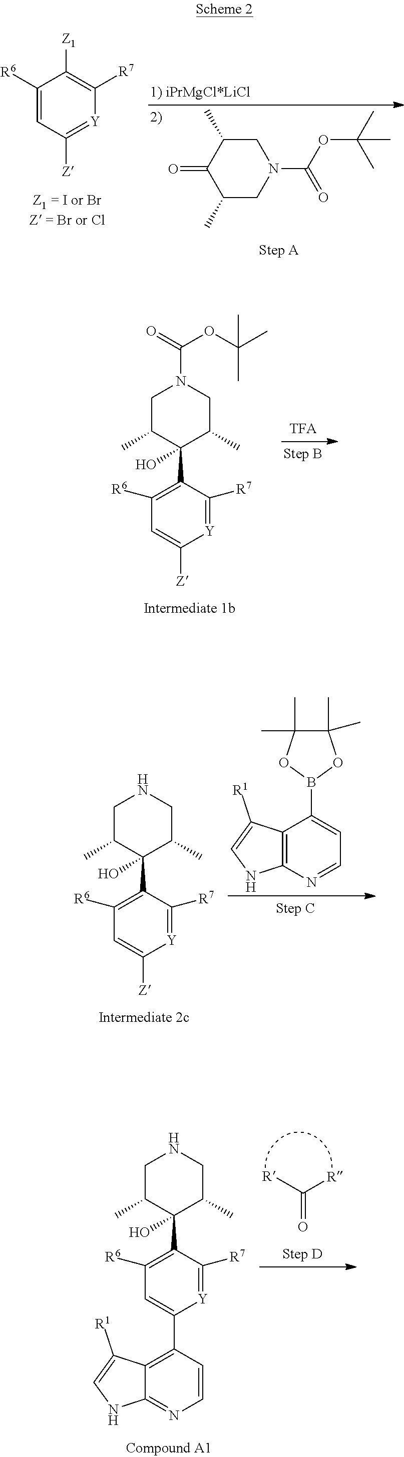

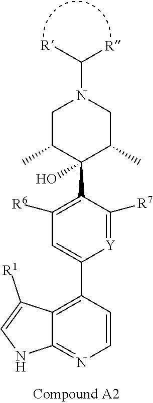

### Synthesis of Intermediates and Compounds of Scheme 2

### Intermediate 2c-1 (Scheme 2): (3S,4s,5R)-4-(4-bromo-2-chlorophenyl)-3,5-dimethylpiperidin-4-ol

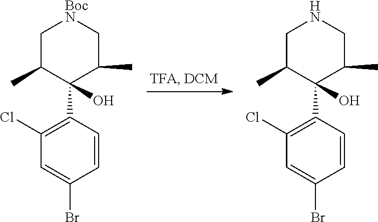 [SMILES: C[C@@H]1CN(C(=O)OC(C)(C)C)C[C@H](C)[C@@]1(O)c1ccc(Br)cc1Cl ; C[C@@H]1CNC[C@H](C)[C@@]1(O)c1ccc(Br)cc1Cl]

Step B: A solution containing tert-butyl (3S,4s,5R)-4-(4-bromo-2-chlorophenyl)-4-hydroxy-3,5-dimethylpiperidine-1-carboxylate (0.200 g, 0.478 mmol) in dichloromethane (1.0 mL) was treated with trifluoroacetic acid (1.0 mL, 28.6 mmol) at 23° C. for 0.5 h. The reaction mixture was concentrated under diminished pressure. The residue was dissolved into 10 mL of ethyl acetate. The resulting solution was treated with 5 mL of saturated aqueous sodium bicarbonate. It was then repeatedly extracted with ethyl acetate (3×, 10 mL portions), the organic phases were combined, and the solvent removed under reduced pressure to give the title product. LC-MS (m/z [M+H]⁺): 320.0.

### Intermediates 2c-2 to 2c-14 (Scheme 2)

Intermediates 2c-2 to 2c-16 below were prepared using an analogous procedure to that of Intermediate 1c-1, and are shown in Table 5.

TABLE 5

| Compound | Structure | IUPAC name | LCMS m/z |
|---|---|---|---|
| 2c-2 | 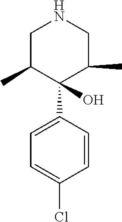 | (3S,4s,5R)-4-(4-chlorophenyl)-3,5- dimethylpiperidin-4-ol | [M + H]⁺: 240.1 |
| 2c-3 | 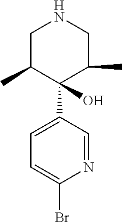 | (3S,4s,5R)-4-(6-bromopyridin-3-yl)-3,5- dimethylpiperidin-4-ol | [M + H]⁺: 286.9 |
| 2c-4 | 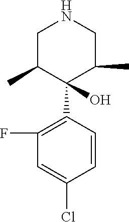 [SMILES: C[C@@H]1CNC[C@H](C)[C@@]1(O)c1ccc(Cl)cc1F] | (3S,4s,5R)-4-(4-chloro-2-fluorophenyl)- 3,5-dimethylpiperidin-4-ol | [M + H]⁺: 258.6 |
| 2c-5 | 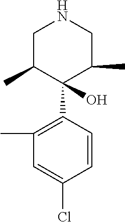 | (3S,4s,5R)-4-(4-chloro-2- methylphenyl)-3,5-dimethylpiperidin-4- ol | [M + H]⁺: 254.1 |
| 2c-6 | 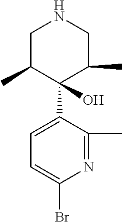 | (3S,4s,5R)-4-(6-bromo-2-methylpyridin- 3-yl)-3,5-dimethylpiperidin-4-ol | [M + H]⁺: 299.0 |
| 2c-7 | 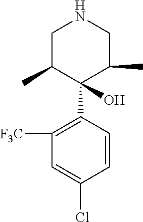 [SMILES: C[C@@H]1CNC[C@H](C)[C@@]1(O)c1ccc(Cl)cc1C(F)(F)F] | (3S,4s,5R)-4-(4-chloro-2- (trifluoromethyl)phenyl)-3,5- dimethylpiperidin-4-ol | [M + H]⁺: 308.1 |
| 2c-8 | 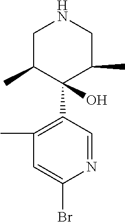 | (3S,4s,5R)-4-(4-chloro-2- methylphenyl)-3,5-dimethylpiperidin-4- ol | [M + H]⁺: 301.1 |
| 2c-9 | 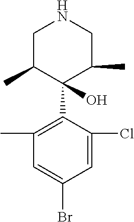 [SMILES: Cc1cc(Br)cc(Cl)c1[C@@]1(O)[C@H](C)CNC[C@@H]1C] | (3S,4s,5R)-4-(4-bromo-2-chloro-6- methylpheny1)-3,5-dimethylpiperidin-4- ol | [M + H]⁺: 334.0 |
| 2c-10 | 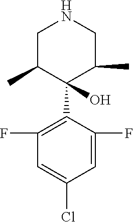 [SMILES: C[C@@H]1CNC[C@H](C)[C@@]1(O)c1c(F)cc(Cl)cc1F] | (3S,4s,5R)-4-(4-bromo-2,6- difluorophenyl)-3,5-dimethylpiperidin- 4-ol | [M + H]⁺: 276.0 |
| 2c-11 | 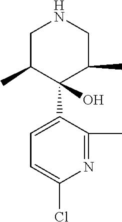 | (3S,4s,5R)-4-(6-chloro-2-methylpyridin- 3-yl)-3,5-dimethylpiperidin-4-ol | [M + H]⁺: 255.1 |
| 2c-12 | 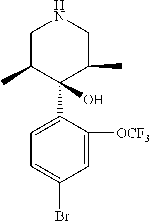 [SMILES: C[C@@H]1CNC[C@H](C)[C@@]1(O)c1ccc(Br)cc1OC(F)(F)F] | (3S,4s,5R)-4-(4-bromo-2- (trifluoromethoxy)phenyl)-3,5- dimethylpiperidin-4-ol | [M + H]⁺: 370.0 |
| 2c-13 | 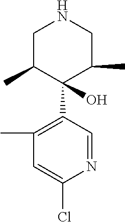 | (3S,4s,5R)-4-(6-chloro-4-methylpyridin- 3-yl)-3,5-dimethylpiperidin-4-ol | [M + H]⁺: 255.1 |
| 2c-14 | 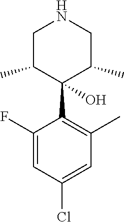 | (3S,4s,5R)-4-(4-chloro-2-fluoro-6- methylphenyl)-3,5-dimethylpiperidin-4- ol | [M + H]⁺: 272.1 |
| 2c-15 | 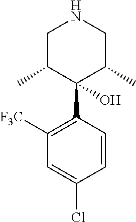 | (3R,4s,5S)-4-(4-chloro-2- (trifluoromethyl)phenyl)-3,5- dimethylpiperidin-4-ol | [M + H]⁺: 308.1 |
| 2c-16 | 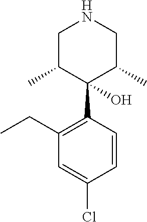 [SMILES: CCc1cc(Cl)ccc1[C@@]1(O)[C@H](C)CNC[C@@H]1C] | (3R,4s,5S)-4-(4-chloro-2-ethylphenyl)- 3,5-dimethylpiperidin-4-ol | [M + H]⁺: 268.1 |

### Compound 7

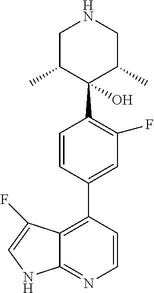 [SMILES: C[C@@H]1CNC[C@H](C)[C@@]1(O)c1ccc(-c2ccnc3[nH]cc(F)c23)cc1F]

### (3S,4s,5R)-4-(2-fluoro-4-(3-fluoro-1H-pyrrolo[2,3-b]pyridin-4-yl)phenyl)-3,5-dimethyl piperidin-4-ol

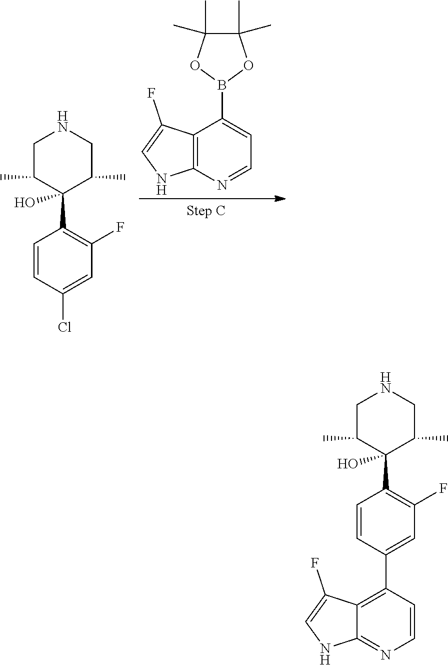

Step C: A microwave vial was charged with (3S,5R)-4-(4-chloro-2-fluorophenyl)-3,5-dimethylpiperidin-4-ol (0.078 g, 0.296 mmol), 3-fluoro-4-(4,4,5,5-tetramethyl-1,3,2-dioxaborolan-2-yl)-1H-pyrrolo[2,3-b]pyridine (0.078 g, 0.0296 mmol), potassium phosphate (tribasic, K₃PO₄, 0.100 g, 0.471 mmol), and chloro(2-dicyclohexylphosphino-2′,4′,6′-triisopropyl-1,1′-biphenyl)[2-(2′-amino-1,1′-biphenyl)]palladium(II) (Xphos Pd G2, 15.0 mg, 0.019 mmol). The vial was capped. Then, tetrahydrofuran (1.0 mL) was added, followed by water (1.0 ml). The content of the reaction vial was purged with nitrogen via bubbling through the solvent. The reaction was then heated in the microwave reactor at 100° C. for 45 min. The reaction was cooled, and the solvent removed by rotary evaporation. The crude reaction mixture was dissolved/suspended in methanol (1 ml) and filtered through a nylon syringe filter. Purification by reverse phase chromatography (500 to 100% acetonitrile in water with 0.1% trifluoroacetic acid) gave the title product as the TFA salt. H NMR (400 MHz, DMSO-d₆) δ 11.70 (d, J=2.8 Hz, 1H), 8.64 (s, 2H), 8.32 (d, J=4.9 Hz, 1H), 7.71 (t, J=8.3 Hz, 1H), 7.60-7.51 (i, 2H), 7.47 (d, J=13.3 Hz, 1H), 7.21 (d, J=4.9 Hz, 1H), 5.40 (s, 1H), 3.12 (d, J=12.0 Hz, 2H), 2.93 (d, J=12.1 Hz, 3H), 2.61 (dq, J=11.8, 6.8, 4.8 Hz, 2H), 0.62 (d, J=6.8 Hz, 6H). ¹⁹F NMR (376 MHz, DMSO-d₆) δ−73.900(s), −112.60 (s), −164.92 (s) (TFA salt). LC-MS (m/z [M+H]⁺): 358.2

### Compounds 8-16

Compounds 8-16 and 300-303 were prepared using an analogous procedure to that of Compound 7, and are shown in Table 6.

TABLE 6

| | | | | LCMS |
|---|---|---|---|---|
| Compound | Structure | IUPAC name | ¹H NMR | m/z |
| 8 | 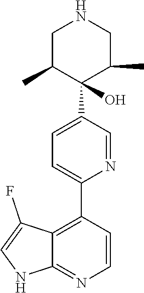 [SMILES: C[C@@H]1CNC[C@H](C)[C@@]1(O)c1ccc(-c2ccnc3[nH]cc(F)c23)nc1] | (3S,4s,5R)-4-(6-(3- fluoro-1H- pyrrolo[2,3- b]pyridin-4- yl)pyridin-3-yl)-3,5- dimethylpiperidin-4- ol | ¹H NMR (400 MHz, DMSO-d₆) δ 11.73 (s, 1H), 8.72 − 8.52 (m, 2H), 8.37 (d, J = 4.9 Hz, 1H), 7.93 − 7.82 (m, 1H), 7.59 (d, J = 2.9 Hz, 1H), 7.48 (d, J = 4.9 Hz, 1H), 5.39 (s, 1H), 3.18 (d, J = 12.2 Hz, 2H), 2.95 (q, J = 11.9 Hz, 2H), 2.43 − 2.28 (m, 2H), 0.61 (d, J = 6.8 Hz, 6H). | [M + H]⁺: 341.2 |
| 9 | 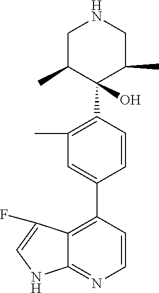 [SMILES: Cc1cc(-c2ccnc3[nH]cc(F)c23)ccc1[C@@]1(O)[C@H](C)CNC[C@@H]1C] | (3S,4s,5R)-4-(4-(3- fluoro-1H- pyrrolo[2,3- b]pyridin-4-yl)-2- methylphenyl)-3,5- dimethylpiperidin-4- ol | ¹H NMR (400 MHz, DMSO-d₆) δ 11.64 (s, 1H), 8.66 − 8.46 (m, 2H), 8.31 (d, J = 4.8 Hz, 1H), 7.80 (d, J = 8.2 Hz, 1H), 7.52 (t, J = 2.4 Hz, 2H), 7.17 (d, J = 4.9 Hz, 1H), 5.15 (s, 1H), 3.11 (d, J = 11.8 Hz, 2H), 2.98 (q, J = 12.1 Hz, 2H), 2.75 − 2.65 (m, 2H), 2.55 (s, 3H), 0.59 (d, J = 6.8 Hz, 6H). | [M + H]⁺: 354.3 |
| 10 | 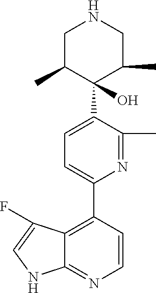 [SMILES: Cc1nc(-c2ccnc3[nH]cc(F)c23)ccc1[C@@]1(O)[C@H](C)CNC[C@@H]1C] | (3S,4s,5R)-4-(6-(3- fluoro-1H- pyrrolo[2,3- b]pyridin-4-yl)-2- methylpyridin-3-yl)- 3,5- dimethylpiperidin-4- ol | ¹H NMR (400 MHz, DMSO-d₆) δ 11.70 (s, 1H), 8.36 (d, J = 5.0 Hz, 1H), 8.16 (d, J = 8.2 Hz, 1H), 7.74 (dd, J = 8.2, 3.5 Hz, 1H), 7.57 (t, J = 2.5 Hz, 1H), 7.49 (dd, J = 4.9, 1.9 Hz, 1H), 5.30 (s, 1H), 3.14 (d, J = 11.9 Hz, 2H), 2.99 (d, J = 11.2 Hz, 2H), 2.81 − 2.69 (m, 2H) 2.76 (s, 3H), 0.60 (d, J = 6.8 Hz, 6H). | [M + H]⁺: 355.2 |
| 11 | 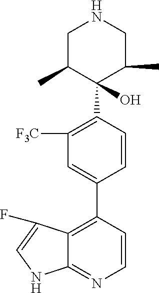 [SMILES: C[C@@H]1CNC[C@H](C)[C@@]1(O)c1ccc(-c2ccnc3[nH]cc(F)c23)cc1C(F)(F)F] | (3S,4s,5R)-4-(4-(3- fluoro-1H- pyrrolo[2,3- b]pyridin-4-yl)-2- (trifluoromethyl)phen yl)-3,5- dimethylpiperidin-4- ol | | [M + H]⁺: 408.1 |
| 12 | 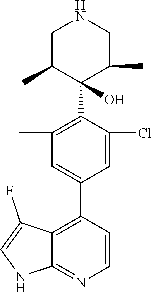 [SMILES: Cc1cc(-c2ccnc3[nH]cc(F)c23)cc(Cl)c1[C@@]1(O)[C@H](C)CNC[C@@H]1C] | (3S,4s,5R)-4-(2- chloro-4-(3-fluoro- 1H-pyrrolo[2,3- b]pyridin-4-yl)-6- methylphenyl)-3,5- dimethylpiperidin-4- ol | ¹H NMR (400 MHz, DMSO-d₆) δ 11.66 (s, 1H), 8.68 − 8.57 (m, 2H), 8.32 (d, J = 4.9 Hz, 1H), 7.96 (t, J = 2.8 Hz, 1H), 7.60 (s, 1H), 7.52 (t, J = 2.5 Hz, 1H), 7.17 (d, J = 4.9 Hz, 1H), 5.27 (s, 1H), 3.26 − 3.18 (m, 2H), 3.11 (d, J = 12.0 Hz, 2H), 2.99 − 2.86 (m, 2H), 2.44 (s, 3H), 0.57 (d, J = 6.9 Hz, 6H). | [M + H]⁺: 388.2 |
| 13 | 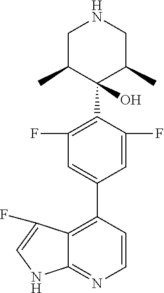 [SMILES: C[C@@H]1CNC[C@H](C)[C@@]1(O)c1c(F)cc(-c2ccnc3[nH]cc(F)c23)cc1F] | (3S,4s,5R)-4-(2- fluoro-4-(3-fluoro- 1H-pyrrolo[2,3- b]pyridin-4- yl)phenyl)-3,5- dimethyl piperidin-4- ol | ¹H NMR (400 MHz, DMSO-d₆) δ 11.79 (s, 1H), 8.60 (s, 2H), 8.35 (d, J = 4.9 Hz, 1H), 7.60 (dd, J = 2.9, 1.9 Hz, 1H), 7.39 (t, J = 11.0 Hz, 2H), 7.26 (d, J = 4.9 Hz, 1H), 5.43 (s, 1H), 3.09 (d, J = 12.0 Hz, 2H), 3.00 − 2.90 (m, 2H), 2.61 (dt, J = 11.7, 5.4 Hz, 2H), 0.82 (d, J = 6.9 Hz, 6H). | [M + H]⁺: 376.2 |
| 14 | 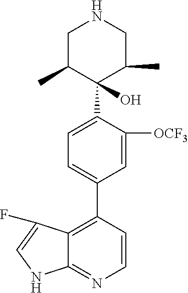 [SMILES: C[C@@H]1CNC[C@H](C)[C@@]1(O)c1ccc(-c2ccnc3[nH]cc(F)c23)cc1OC(F)(F)F] | (3S,4s,5R)-4-(4-(3- fluoro-1H- pyrrolo[2,3- b]pyridin-4-yl)-2- (trifluoromethoxy) phenyl)-3,5- dimethylpiperidin-4- ol | ¹H NMR (400 MHz, DMSO-d₆) δ 11.77 (s, 1H), 8.64 (s, 1H), 8.36 (d, J = 4.9 Hz, 1H), 7.90 (d, J = 8.2 Hz, 1H), 7.73 (d, J = 8.2 Hz, 1H), 7.59 (s, 2H), 7.31 − 7.10 (m, 1H), 5.52 (s, 1H), 3.14 (d, J = 11.6 Hz, 2H), 3.04 − 2.92 (m, 2H), 2.69 − 2.54 (m, 2H), 0.61 (d, J = 6.9 Hz, 6H). | [M + H]⁺: 424.2 |
| 15 | 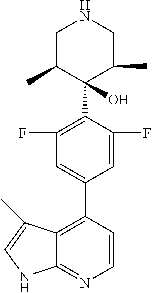 [SMILES: Cc1c[nH]c2nccc(-c3cc(F)c([C@@]4(O)[C@H](C)CNC[C@@H]4C)c(F)c3)c12] | (3S,4s,5R)-4-(2,6- difluoro-4-(3-fluoro- 1H-pyrrolo[2,3- b]pyridin-4- yl)phenyl)-3,5- dimethylpiperidin-4- ol | ¹H NMR (400 MHz, DMSO-d₆) δ 11.56 (s, 1H), 8.58 (s, 2H), 8.22 (d, J = 4.9 Hz, 1H), 7.31 (dd, J = 2.5, 1.2 Hz, 1H), 7.19 (t, J = 11.6 Hz, 2H), 6.96 (d, J = 4.8 Hz, 1H), 5.40 (s, 1H), 3.08 (d, J = 12.0 Hz, 2H), 2.95 (s, 3H), 2.74 − 2.56 (m, 2H), 1.89 (d, J = 1.1 Hz, 3H), 0.82 (d, J = 6.8 Hz, 6H). | [M + H]⁺: 372.2 |
| 16 | 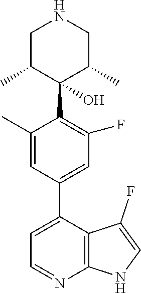 [SMILES: Cc1cc(-c2ccnc3[nH]cc(F)c23)cc(F)c1[C@@]1(O)[C@H](C)CNC[C@@H]1C] | (3S,4s,5R)-4-(2- fluoro-4-(3-fluoro- 1H-pyrrolo[2,3- b]pyridin-4-yl)-6- methylphenyl)-3,5- dimethylpiperidin-4- ol | ¹H NMR (400 MHz, DMSO-d₆) δ 11.71 (s, 1H), 8.62 (s, 2H), 8.35 (dd, J = 22.3, 4.8 Hz, 1H), 7.56 (t, J = 2.5 Hz, 1H), 7.32 (d, J = 9.5 Hz, 2H), 7.27 − 7.06 (m, 1H), 4.94 (s, 1H), 3.09 (d, J = 11.9 Hz, 2H), 3.02 − 2.97 (m, 2H), 2.78 − 2.71 (m, 2H), 2.71 (s, 3H), 0.77 (d, J = 6.8 Hz, 6H). | [M + H]⁺: 372.2 |
| 300 | 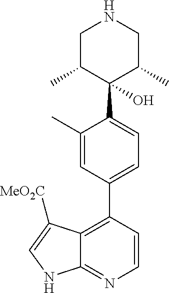 [SMILES: COC(=O)c1c[nH]c2nccc(-c3ccc([C@@]4(O)[C@H](C)CNC[C@@H]4C)c(C)c3)c12] | methyl 4-(4- ((3S,4s,5R)-4- hydroxy-3,5- dimethylpiperidin-4- yl)-3-methylphenyl)- 1H-pyrrolo[2,3- b]pyridine-3- carboxylate | ¹H NMR (400 MHz, DMSO-d₆) δ 12.56 (s, 1H), 8.57 (bs, 2H), 8.34 (d, J = 4.9 Hz, 1H), 8.18 (d, J = 3.1 Hz, 1H), 7.72 (d, J = 8.2 Hz, 1H), 7.27 − 7.17 (m, 2H), 7.17 − 7.10 (m, 1H), 5.10 (s, 1H), 3.25 (s, 3H), 3.14 − 3.06 (m, 2H), 3.05 − 2.92 (m, 2H), 2.75 − 2.65 (m, 1H), 2.43 − 2.33 (m, 1H), 0.67 (dd, J = 45.1, 6.8 Hz, 6H). | [M + H]⁺: 394.1 |
| 301 | 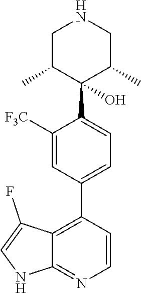 [SMILES: C[C@@H]1CNC[C@H](C)[C@@]1(O)c1ccc(-c2ccnc3[nH]cc(F)c23)cc1C(F)(F)F] | (3S,4s,5R)-4-(4-(3- fluoro-1H- pyrrolo[2,3- b]pyridin-4-yl)-2- (trifluoromethyl)phen yl)-3,5- dimethylpiperidin-4- ol | ¹H NMR (400 MHz, DMSO-d₆) δ 11.75 (s, 1H), 8.65 (bs, 1H), 8.52 (bs, 1H), 8.36 (d, J = 4.9 Hz, 1H), 8.08 − 7.97 (m, 2H), 7.57 (dd, J = 5.9, 3.3 Hz, 2H), 7.27 (d, J = 5.1 Hz, 1H), 4.93 (s, 1H), 3.19 − 2.11 (m, 2H), 2.98 (q, J = 11.8 Hz, 2H), 2.48 − 2.42 (m, 2H), 0.62 (t, J = 6.6 Hz, 7H). | [M + H]⁺: 408.1 |
| 302 | 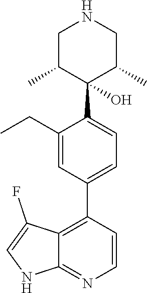 [SMILES: CCc1cc(-c2ccnc3[nH]cc(F)c23)ccc1[C@@]1(O)[C@H](C)CNC[C@@H]1C] | (3S,4s,5R)-4-(2- ethyl-4-(3-fluoro-1H- pyrrolo[2,3- b]pyridin-4- yl)phenyl)-3,5- dimethylpiperidin-4- ol | ¹H NMR (400 MHz, DMSO-d₆) δ 11.64 (s, 1H), 8.31 (d, J = 4.9 Hz, 1H), 7.77 (d, J = 8.3 Hz, 1H), 7.61 − 7.42 (m, 3H), 7.29 − 7.03 (m, 1H), 5.10 (s, 1H), 3.13 (d, J = 12.5 Hz, 2H), 3.03 − 2.83 (m, 4H), 2.67 − 2.56 (m, 2H), 1.22 (t, J = 7.4 Hz, 3H), 0.59 (d, J = 6.7 Hz, 6H). | [M + H]⁺: 368.1 |
| 303 | 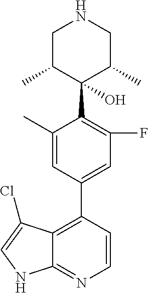 [SMILES: Cc1cc(-c2ccnc3[nH]cc(Cl)c23)cc(F)c1[C@@]1(O)[C@H](C)CNC[C@@H]1C] | (3S,4s,5R)-4-(4-(3- chloro-1H- pyrrolo[2,3- b]pyridin-4-yl)-2- fluoro-6- methylphenyl)-3,5- dimethylpiperidin-4- ol | ¹H NMR (400 MHz, DMSO-d₆) δ 12.19 (d, J = 2.8 Hz, 1H), 8.59 (s, 2H), 8.32 (d, J = 4.8 Hz, 1H), 7.71 (d, J = 2.8 Hz, 1H), 7.24 − 6.99 (m, 3H), 4.91 (s, 1H), 3.12 − 3.05 (m, 2H), 3.01 − 2.90 (m, 2H), 2.76 − 2.69 (m, 2H), 2.68 (s, 3H), 0.76 (d, J = 6.8 Hz, 6H). | [M + H]⁺: 388.0 |

### Compound 17

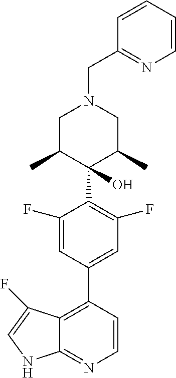 [SMILES: C[C@@H]1CN(Cc2ccccn2)C[C@H](C)[C@@]1(O)c1c(F)cc(-c2ccnc3[nH]cc(F)c23)cc1F]

### (3S,4s,5R)-4-(2,6-difluoro-4-(3-fluoro-1H-pyrrolo[2,3-b]pyridin-4-yl)phenyl)-3,5-dimethyl-1-(pyridin-2-ylmethyl)piperidin-4-ol

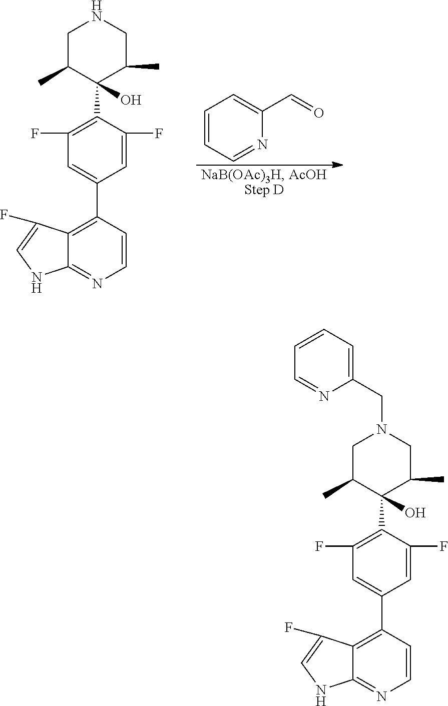

Step D: To a solution of (3S,4s,5R)-4-(2-fluoro-4-(3-fluoro-1H-pyrrolo[2,3-b]pyridin-4-yl)phenyl)-3,5-dimethyl piperidin-4-ol (0.050 g, 0.133 mmol) in methanol (1.0 ml) was added acetic acid (0.008 g, 0.133 mmol), followed by picolinaldehyde (0.016 g, 0.149 mmol) The mixture was heated to 50° C. for 20 minutes via microwave irradiation. The mixture was then cooled to room temperature and sodium triacetoxyborohydride (0.100 g, 0.472 mmol) was added as a solid. The reaction was then stirred at room temperature for 1 hour. The reaction was quenched by being poured into saturated aqueous sodium bicarbonate, and extracted into ethyl acetate (2×). The organic phases were partitioned and combined, and the solvent removed by rotary evaporation to give the crude product. The crude product was diluted with 1 mL of methanol, was then filtered, and the collected solution was purified by reverse-phase HPLC to provide the title compound. LC-MS (m/z [M+H]⁺): 467.2

### Compounds 18-36

Compounds 18-36 were prepared using an analogous procedure to that of Compound 17, and are shown in Table 7.

TABLE 7

| | | | | LCMS |
|---|---|---|---|---|
| Compound | Structure | IUPAC name | ¹H NMR | m/z |
| 18 | 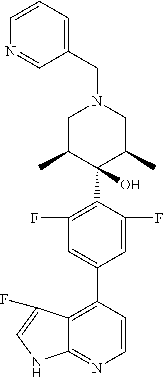 [SMILES: C[C@@H]1CN(Cc2cccnc2)C[C@H](C)[C@@]1(O)c1c(F)cc(-c2ccnc3[nH]cc(F)c23)cc1F] | (3S,4s,5R)-4-(2,6- difluoro-4-(3- fluoro-1H- pyrrolo[2,3- b]pyridin-4- yl)phenyl)-3,5- dimethyl-1- (pyridin-3- ylmethyl)piperidin- 4-ol | | [M + H]⁺: 467.2 |
| 19 | 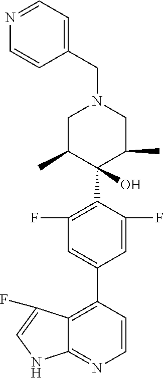 [SMILES: C[C@@H]1CN(Cc2ccncc2)C[C@H](C)[C@@]1(O)c1c(F)cc(-c2ccnc3[nH]cc(F)c23)cc1F] | (3S,4s,5R)-4-(2,6- difluoro-4-(3- fluoro-1H- pyrrolo[2,3- b]pyridin-4- yl)phenyl)-3,5- dimethyl-1- (pyridin-4- ylmethyl)piperidin- 4-ol | ¹H NMR (400 MHz, DMSO-d₆) δ 11.75 (s, 1H), 8.52 (s, 2H), 8.33 (d, J = 5.0 Hz, 1H), 7.59 (t, J = 2.5 Hz, 1H), 7.34 (d, J = 11.4 Hz, 4H), 7.26 (d, J = 4.9 Hz, 1H), 4.64 (s, 1H), 3.53=7 − 3.48 (m, 2H), 3.28 (s, 2H), 2.48 − 2.42 (m, 2H), 2.35 − 2.22 (m, 2H), 0.74 (d, J = 6.6 Hz, 6H). | [M + H]⁺: 467.2 |
| 20 | 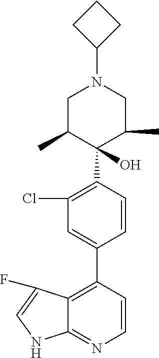 [SMILES: C[C@@H]1CN(C2CCC2)C[C@H](C)[C@@]1(O)c1ccc(-c2ccnc3[nH]cc(F)c23)cc1Cl] | (3S,4s,5R)-4-(2- chloro-4-(3-fluoro- 1H-pyrrolo[2,3- b]pyridin-4- yl)phenyl)-1- cyclobutyl-3,5- dimethylpiperidin- 4-ol | ¹H NMR (400 MHz, DMSO-d₆) δ 11.71 (s, 1H), 8.33 (d, J = 4.9 Hz, 1H), 8.15 (s, 1H), 7.94 (d, J = 8.2 Hz, 1H), 7.72 − 7.60 (m, 2H), 7.55 (d, J = 2.6 Hz, 1H), 7.23 (d, J = 4.9 Hz, 1H), 4.74 (s, 1H), 3.04 − 2.94 (m, 2H), 2.93 − 2.85 (m, 1H), 2.67 − 2.55 (m, 2H), 2.10 (t, J = 11.2 Hz, 2H), 2.04 − 1.97 (m, 2H), 1.95 − 1.83 (m, 2H), 1.69 − 1.59 (m, 2H), 0.54 (d, J = 6.9 Hz, 6H). | [M + H]⁺: 428.2 |
| 21 | 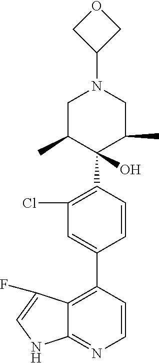 [SMILES: C[C@@H]1CN(C2COC2)C[C@H](C)[C@@]1(O)c1ccc(-c2ccnc3[nH]cc(F)c23)cc1Cl] | (3S,4s,5R)-4-(2- chloro-4-(3-fluoro- 1H-pyrrolo[2,3- b]pyridin-4- yl)phenyl)-3,5- dimethyl-1-(oxetan- 3-yl)piperidin-4-ol | ¹H NMR (400 MHz, DMSO-d₆) δ 11.70 (s, 1H), 8.33 (d, J = 5.0 Hz, 1H), 7.95 (d, J = 8.3 Hz, 1H), 7.72 − 7.63 (m, 2H), 7.56 (s, 1H), 7.23 (d, J = 4.9 Hz, 1H), 4.69 (s, 1H), 4.58 − 4.52 (m, 2H), 4.52 − 4.47 (m, 2H), 3.50 − 3.36 (m, 1H), 3.07 − 2.96 (m, 2H), 2.48 − 2.41 (m, 2H), 2.04 (t, J = 11.0 Hz, 2H), 0.54 (d, J = 6.9 Hz, 6H). | [M + H]⁺: 430.2 |
| 22 | 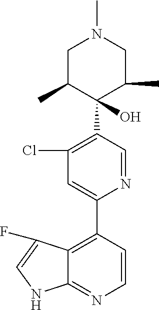 [SMILES: C[C@@H]1CN(C)C[C@H](C)[C@@]1(O)c1cnc(-c2ccnc3[nH]cc(F)c23)cc1Cl] | (3S,4s,5R)-4-(4- chloro-6-(3-fluoro- 1H-pyrrolo[2,3- b]pyridin-4- yl)pyridin-3-yl)- 1,3,5- trimethylpiperidin- 4-ol | ¹H NMR (400 MHz, DMSO-d₆) δ 11.77 (s, 1H), 9.03 (s, 1H), 8.36 (d, J = 4.9 Hz, 1H), 8.14 (s, 1H), 7.86 (d, J = 2.6 Hz, 1H), 7.60 (d, J = 2.6 Hz, 1H), 7.51 (d, J = 4.9 Hz, 1H), 2.93 (p, J = 5.2 Hz, 2H), 2.59 (dd, J = 11.3, 4.0 Hz, 2H), 2.33 − 2.20 (m, 2H), 2.25 (s, 3H), 0.56 (d, J = 6.9 Hz, 6H). | [M + H]⁺: 389.2 |
| 23 | 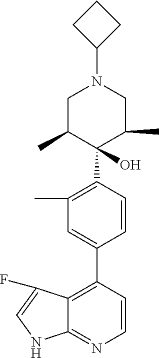 [SMILES: Cc1cc(-c2ccnc3[nH]cc(F)c23)ccc1[C@@]1(O)[C@H](C)CN(C2CCC2)C[C@@H]1C] | (3S,4s,5R)-1- cyclobutyl-4-(4-(3- fluoro-1H- pyrrolo[2,3- b]pyridin-4-yl)-2- methylphenyl)-3,5- dimethylpiperidin- 4-ol | ¹H NMR (400 MHz, DMSO-d₆) δ 11.60 (s, 1H), 8.27 (t, J = 4.3 Hz, 1H), 8.15 (s, 1H), 7.80 (d, J = 8.2 Hz, 1H), 7.50 − 7.24 (m, 3H), 7.15 (t, J = 4.7 Hz, 1H), 4.52 (s, 1H), 3.00 − 2.83 (m, 1H), 2.91 (d, J = 35.3 Hz, 1H), 2.69 − 2.65 (m, 1H), 2.64 (s, 1H), 2.52 (s, 2H), 2.23 − 2.12 (m, 2H), 2.10 − 1.98 (m, 2H), 1.85 − 1.81 (m, 1H), 1.70 − 1.58 (m, 2H), 0.63 (d, J = 6.5 Hz, 2H), 0.53 (d, J = 6.7 Hz, 4H). | [M + H]⁺: 408.3 |
| 24 | 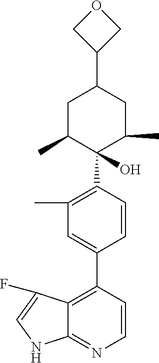 [SMILES: Cc1cc(-c2ccnc3[nH]cc(F)c23)ccc1[C@@]1(O)[C@H](C)CC(C2COC2)C[C@@H]1C] | (3S,4s,5R)-4-(4-(3- fluoro-1H- pyrrolo[2,3- b]pyridin-4-yl)-2- methylphenyl)-3,5- dimethyl-1-(oxetan- 3-yl)piperidin-4-ol | ¹H NMR (400 MHz, DMSO-d₆) δ 11.62 (s, 1H), 8.30 (d, J = 4.6 Hz, 1H), 7.82 (d, J = 8.2 Hz, 1H), 7.55 − 7.33 (m, 3H), 7.17 (t, J = 4.5 Hz, 1H), 4.91 − 4.34 (m, 5H), 2.66 (s, 1H), 2.56 (s, 2H), 2.46 − 2.28 (m, 3H), 2.25 − 2.16 (m, 1H), 2.13 − 1.97 (m, 2H), 0.65 (s, 2H), 0.56 (s, 4H). | [M + H]⁺: 410.3 |
| 25 | 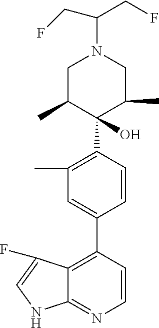 [SMILES: Cc1cc(-c2ccnc3[nH]cc(F)c23)ccc1[C@@]1(O)[C@H](C)CN(C(CF)CF)C[C@@H]1C] | (3S,4s,5R)-1-(1,3- difluoropropan-2- yl)-4-(4-(3-fluoro- 1H-pyrrolo[2,3- b]pyridin-4-yl)-2- methylphenyl)-3,5- dimethylpiperidin- 4-ol | | [M + H]⁺: 432.3 |
| 26 | 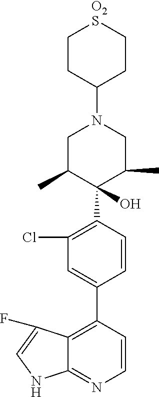 [SMILES: C[C@@H]1CN(C2CCS(=O)(=O)CC2)C[C@H](C)[C@@]1(O)c1ccc(-c2ccnc3[nH]cc(F)c23)cc1Cl] | 4-((3S,4s,5R)-4-(2- chloro-4-(3-fluoro- 1H-pyrrolo[2,3- b]pyridin-4- yl)phenyl)-4- hydroxy-3,5- dimethylpiperidin- 1-yl)tetrahydro-2H- thiopyran 1,1- dioxide | ¹H NMR (400 MHz, DMSO-d₆) δ 11.71 (s, 1H), 8.34 (dd, J = 6.4, 4.9 Hz, 1H), 8.18 (s, 1H), 8.02 − 7.92 (m, 1H), 7.74 − 7.61 (m, 2H), 7.61 − 7.47 (m, 1H), 7.24 (dd, J = 5.0, 2.2 Hz, 1H), 4.65 (s, 1H), 3.20 − 3.04 (m, 5H), 3.03 − 2.92 (m, 1H), 2.76 − 2.64 (m, 2H), 2.49 − 2.41 (m, 2H), 2.07 − 1.99 (m, 5H), 0.55 (d, J = 6.9 Hz, 6H). | [M + H]⁺: 506.2 |
| 27 | 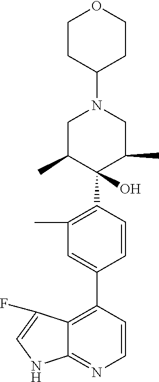 [SMILES: Cc1cc(-c2ccnc3[nH]cc(F)c23)ccc1[C@@]1(O)[C@H](C)CN(C2CCOCC2)C[C@@H]1C] | (3S,4s,5R)-4-(4-(3- fluoro-1H- pyrrolo[2,3- b]pyridin-4-yl)-2- methylphenyl)-3,5- dimethyl-1- (tetrahydro-2H- pyran-4- yl)piperidin-4-ol | ¹H NMR (400 MHz, DMSO-d₆) δ 11.60 (s, 1H), 8.29 (dd, J = 4.9, 3.1 Hz, 1H), 8.17 (s, 1H), 7.83 (d, J = 8.3 Hz, 1H), 7.51 − 7.40 (m, 3H), 7.16 (t, J = 4.9 Hz, 1H), 4.42 (s, 1H), 3.91 (d, J = 11.1 Hz, 2H), 2.69 − 2.60 (m, 1H), 2.67 (s, 1H), 2.54 (s, 2H), 2.57 − 2.30 (m, 2H), 2.21 − 2.11 (m, 1H), 1.79 − 1.72 (m, 2H) 1.56 − 1.44 (m, 2H), 0.66 (d, J = 6.8 Hz, 2H), 0.55 (d, J = 6.4 Hz, 4H). | [M + H]⁺: 438.3 |
| 28 | 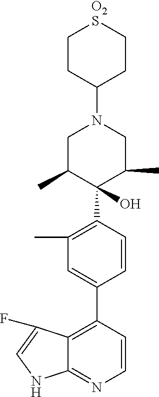 [SMILES: Cc1cc(-c2ccnc3[nH]cc(F)c23)ccc1[C@@]1(O)[C@H](C)CN(C2CCS(=O)(=O)CC2)C[C@@H]1C] | 4-((3S,4s,5R)-4-(4- (3-fluoro-1H- pyrrolo[2,3- b]pyridin-4-yl)-2- methylphenyl)-4- hydroxy-3,5- dimethylpiperidin- 1-yl)tetrahydro-2H- thiopyran 1,1- dioxide | ¹H NMR (400 MHz, DMSO-d₆) δ 11.58 (s, 1H), 8.27 (dd, J = 4.9, 2.4 Hz, 1H), 7.82 (d, J = 8.3 Hz, 1H), 7.54 − 7.35 (m, 3H), 7.15 (t, J = 5.0 Hz, 1H), 4.33 (s, 1H), 4.10 − 4.04 (m, 1H), 3.17 − 3.01 (m, 5H), 2.65 (s, 1H), 2.52 (s, 2H), 2.44 − 2.29 (m, 1H), 2.21 − 2.07 (m, 4H), 2.05 − 1.98 (m, 4H), 0.63 (d, J = 6.8 Hz, 2H), 0.53 (d, J = 6.1 Hz, 4H). | [M + H]⁺: 486.3 |
| 29 | 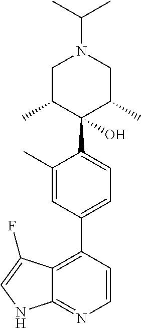 [SMILES: Cc1cc(-c2ccnc3[nH]cc(F)c23)ccc1[C@@]1(O)[C@H](C)CN(C(C)C)C[C@@H]1C] | (3S,4s, 5R)-4-(4-(3- fluoro-1H- pyrrolo[2,3- b]pyridin-4-yl)-2- methylphenyl)-1- isopropyl-3,5- dimethylpiperidin- 4-ol | ¹H NMR (400 MHz, DMSO-d₆) δ 11.60 (s, 1H), 8.29 (dd, J = 4.9, 3.3 Hz, 1H), 8.20 (s, 1H), 7.82 (d, J = 8.3 Hz, 1H), 7.55 − 7.28 (m, 4H), 7.16 (dd, J = 4.9, 4.0 Hz, 1H), 4.54 (s, 1H), 2.99 − 9.89 (m, 3H), 2.66 (d, J = 2.7 Hz, 1H), 2.62 − 2.56 (m, 2H), 2.54 (s, 2H), 2.28 − 2.16 (m, 1H), 1.08 (dd, J = 8.8, 6.5 Hz, 7H), 0.66 (d, J = 6.8 Hz, 2H), 0.56 (d, J = 5.8 Hz, 4H). | [M + H]⁺: 396.3 |
| 30 | 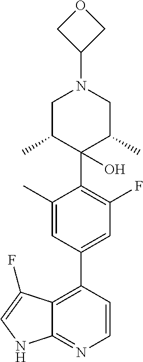 [SMILES: Cc1cc(-c2ccnc3[nH]cc(F)c23)cc(F)c1C1(O)[C@H](C)CN(C2COC2)C[C@@H]1C] | (3S,5R)-4-(2- fluoro-4-(3-fluoro- 1H-pyrrolo[2,3- b]pyridin-4-yl)-6- methylphenyl)-3,5- dimethyl-1-(oxetan- 3-yl)piperidin-4-ol | ¹H NMR (400 MHz, DMSO-d₆) δ 11.67 (s, 1H), 8.31 (d, J = 4.9 Hz, 1H), 7.54 (s, 1H), 7.25 (d, J = 6.8 Hz, 3H), 7.21 (dd, J = 4.9, 1.2 Hz, 1H), 4.58 − 4.52 (m, 2H), 4.52 − 4.45 (m, 3H), 4.24 − 4.16 (m, 1H), 3.49 − 3.38 (m, 1H), 2.70 (s, 3H), 2.63 − 2.54 (m, 1H), 2.44 − 2.32 (m, 2H), 2.07 − 1.97 (m, 2H), 0.70 (d, J = 6.8 Hz, 6H). | [M + H]⁺: 428.2 |
| 31 | 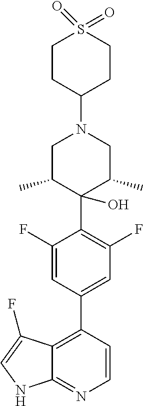 [SMILES: C[C@@H]1CN(C2CCS(=O)(=O)CC2)C[C@H](C)C1(O)c1c(F)cc(-c2ccnc3[nH]cc(F)c23)cc1F] | 4-((3S,5R)-4-(2,6- difluoro-4-(3- fluoro-1H- pyrrolo[2,3- b]pyridin-4- yl)phenyl)-4- hydroxy-3,5- dimethylpiperidin- 1-yl)tetrahydro-2H- thiopyran 1,1- dioxide | ¹H NMR (400 MHz, DMSO-d₆) δ 11.76 (s, 1H), 8.33 (d, J = 4.9 Hz, 1H), 7.59 (s, 1H), 7.32 (t, J = 10.6 Hz, 2H), 7.26 (d, J = 4.9 Hz, 1H), 4.61 (s, 1H), 3.19 − 3.05 (m, 4H), 2.70 − 2.63 (m, 1H), 2.47 − 2.39 (m, 6H), 2.06 − 1.98 (m, 4H), 0.77 (d, J = 4.7 Hz, 6H). | [M + H]⁺: 508.2 |
| 32 | 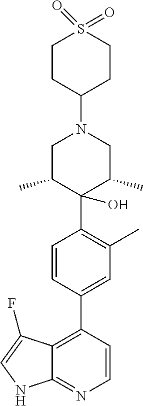 [SMILES: Cc1cc(-c2ccnc3[nH]cc(F)c23)ccc1C1(O)[C@H](C)CN(C2CCS(=O)(=O)CC2)C[C@@H]1C] | 4-((3S,5R)-4-(6-(3- fluoro-1H- pyrrolo[2,3- b]pyridin-4-yl)-2- methylpyridin-3- yl)-4-hydroxy-3,5- dimethylpiperidin- 1-yl)tetrahydro-2H- thiopyran 1,1- dioxide | | [M + H]⁺: 487.2 |
| 33 | 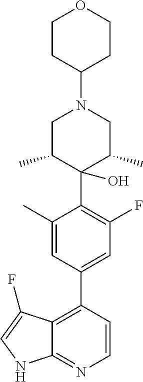 [SMILES: Cc1cc(-c2ccnc3[nH]cc(F)c23)cc(F)c1C1(O)[C@H](C)CN(C2CCOCC2)C[C@@H]1C] | (3S,5R)-4-(2- fluoro-4-(3-fluoro- 1H-pyrrolo[2,3- b]pyridin-4-yl)-6- methylphenyl)-3,5- dimethyl-1- (tetrahydro-2H- pyran-4- yl)piperidin-4-ol | ¹H NMR (400 MHz, DMSO-d₆) δ 11.92 − 11.50 (m, 1H), 8.30 (d, J = 4.9 Hz, 1H), 8.15 (s, 1H), 7.54 (s, 1H), 7.37 − 7.09 (m, 3H), 4.25 (s, 1H), 4.07 − 3.78 (m, 2H), 3.31 − 3.24 (m, 3H), 2.70 (s, 3H), 2.68 − 2.53 (m, 3H), 2.46 − 2.40 (m, 2H), 1.76 (d, J = 12.5 Hz, 2H), 1.59 − 1.38 (m, 2H), 0.71 (d, J = 6.6 Hz, 6H). | [M + H]⁺: 456.3 |
| 34 | 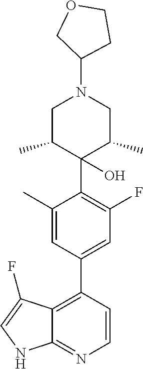 [SMILES: Cc1cc(-c2ccnc3[nH]cc(F)c23)cc(F)c1C1(O)[C@H](C)CN(C2CCOC2)C[C@@H]1C] | (3S,5R)-4-(2- fluoro-4-(3-fluoro- 1H-pyrrolo[2,3- b]pyridin-4-yl)-6- methylphenyl)-3,5- dimethyl-1- (tetrahydrofuran-3- yl)piperidin-4-ol | ¹H NMR (400 MHz, DMSO-d₆) δ 11.68 (s, 1H), 8.30 (d, J = 4.5 Hz, 2H), 7.54 (s, 1H), 7.37 − 7.01 (m, 3H), 4.17 (s, 1H), 3.83 − 3.72 (m, 2H), 3.65 (q, J = 8.3 Hz, 1H), 3.52 (t, J = 7.6 Hz, 1H), 2.92 (t, J = 7.3 Hz, 1H), 2.70 (s, 3H), 2.58 − 2.53 (m, 3H), 2.35 (t, J = 11.0 Hz, 1H), 2.23 (q, J = 10.7 Hz, 2H), 2.04 − 1.93 (m, 1H), 1.84 − 1.73 (m, 1H), 0.69 (t, J = 6.4 Hz, 6H). | [M + H]⁺: 442.2 |
| 35 | 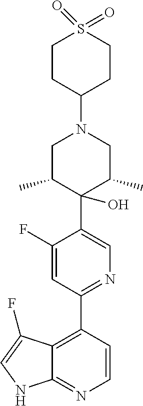 [SMILES: C[C@@H]1CN(C2CCS(=O)(=O)CC2)C[C@H](C)C1(O)c1cnc(-c2ccnc3[nH]cc(F)c23)cc1F] | 4-((3S,5R)-4-(4- fluoro-6-(3-fluoro- 1H-pyrrolo[2,3- b]pyridin-4- yl)pyridin-3-yl)-4- hydroxy-3,5- dimethylpiperidin- 1-yl)tetrahydro-2H- thiopyran 1,1- dioxide | | [M + H]⁺: 491.2 |
| 36 | 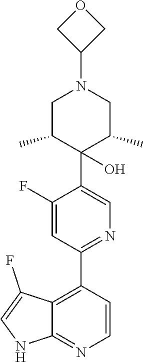 [SMILES: C[C@@H]1CN(C2COC2)C[C@H](C)C1(O)c1cnc(-c2ccnc3[nH]cc(F)c23)cc1F] | (3S,5R)-4-(4- fluoro-6-(3-fluoro- 1H-pyrrolo[2,3- b]pyridin-4- yl)pyridin-3-yl)- 3,5-dimethyl-1- (oxetan-3- yl)piperidin-4-ol | | [M + H]⁺: 415.2 |

### Alternate Synthesis of Compounds of General Formula A1 and A2

Compounds of general formula A1 and A2 can also be prepared as illustrated in Scheme 3 below. Intermediates 1b are synthesized as per Scheme 1, and intermediates 2c are synthesized as per Scheme 2.

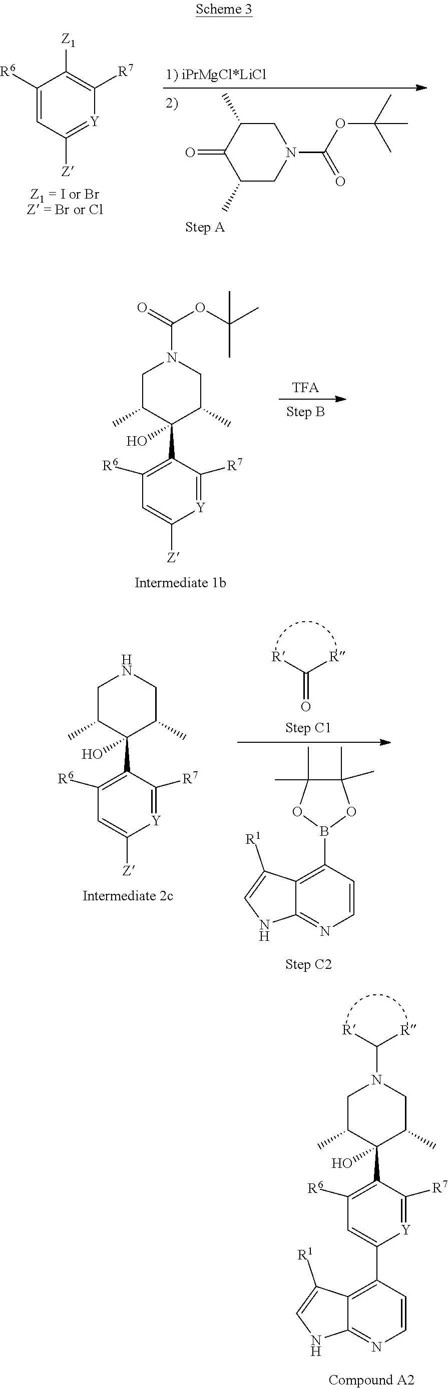

### Synthesis of Compounds of Scheme 3

### Compound 37

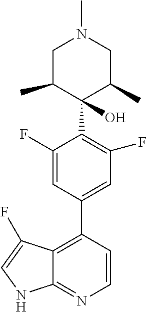 [SMILES: C[C@@H]1CN(C)C[C@H](C)[C@@]1(O)c1c(F)cc(-c2ccnc3[nH]cc(F)c23)cc1F]

### (3S,4s,5R)-4-(2,6-difluoro-4-(3-fluoro-1H-pyrrolo[2,3-b]pyridin-4-yl)phenyl)-1,3,5-trimethylpiperidin-4-ol

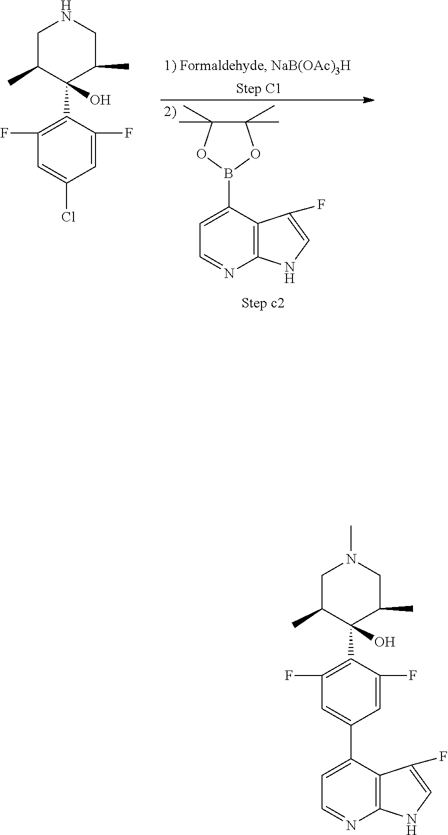 [SMILES: C[C@@H]1CNC[C@H](C)[C@@]1(O)c1c(F)cc(Cl)cc1F ; CC1(C)OB(c2ccnc3[nH]cc(F)c23)OC1(C)C ; C[C@@H]1CN(C)C[C@H](C)[C@@]1(O)c1c(F)cc(-c2ccnc3[nH]cc(F)c23)cc1F]

Step C1: To a solution of (3S,4s,5R)-4-(4-chloro-2,6-difluorophenyl)-3,5-dimethylpiperidin-4-ol (0.100 g, 0.363 mmol) in tetrahydrofuran (5.0 ml) was added formaldehyde (0.147 g, 1.8 mmol, 37% aqueous solution), followed by sodium triacetoxyborohydride (0.154 g, 0.725 mmol). The reaction was stirred at room temperature for 2 hours. The reaction was then poured into a saturated solution of aqueous sodium bicarbonate and extracted with ethyl acetate (2×). The organic phases were partioned and combined, and the solvent removed by rotary evaporation to give the crude product which was used without further purification. LC-MS (m/z [M+H]⁺): 290.1

Step C2: To a 5 ml microwave vial was added potassium phosphate (0.200 g, 0.942 mmol, tribasic), followed by 3-fluoro-4-(4,4,5,5-tetramethyl-1,3,2-dioxaborolan-2-yl)-1H-pyrrolo[2,3-b]pyridine (0.095 g, 0.362 mmol) and Chloro(2-dicyclohexylphosphino-2′,4′,6′-triisopropyl-1,1′-biphenyl)[2-(2′-amino-1,1′-biphenyl)]palladium(II) (Xphos Pd G2, 25.0 mg, 0.032 mmol). The crude product from Step C1 was dissolved in a 1:1 mixture of tetrahydrofuran and water (4 ml total), and added to the 5 ml microwave vial. The vial was sealed and degassed with nitrogen bubbling for 5 minutes. It was then heated to 100° C. for 45 minutes via microwave irradiation. The vial was cooled, and the contents were poured into aqueous sodium bicarbonate. The mixture was extracted with ethyl acetate (2×), the organic phases were partitioned, combined, and the solvent removed by rotary evaporation to give the crude product. The crude product was diluted with 1 mL of methanol, was then filtered, and the collected solution was purified by reverse-phase HPLC to provide the title compound. 1H NMR (400 MHz, DMSO-d₆) δ 11.79 (s, 1H), 9.61 (s, 1H), 8.35 (d, J=4.9 Hz, 1H), 7.60 (dd, J=2.9, 1.9 Hz, 1H), 7.40 (dd, J=13.3, 7.1 Hz, 2H), 7.26 (d, J=4.9 Hz, 1H), 5.53 (s, 1H), 3.24 (d, J=11.6 Hz, 2H), 3.02 (q, J=11.4 Hz, 2H), 2.83 (d, J=4.6 Hz, 3H), 2.73-2.59 (m, 2H), 0.82 (d, J=6.9 Hz, 6H). LC-MS (m/z [M+H]⁺): 390.2

### Compounds 38-44

Compounds 38-44 and 304 were prepared using an analogous procedure to that of Compound 37, and are shown in Table 8.

TABLE 8

| | | | | LCMS |
|---|---|---|---|---|
| Compound | Structure | IUPAC name | ¹H NMR | m/z |
| 38 | 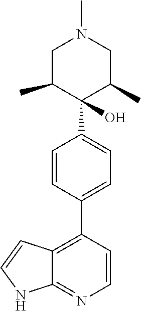 [SMILES: C[C@@H]1CN(C)C[C@H](C)[C@@]1(O)c1ccc(-c2ccnc3[nH]ccc23)cc1] | (3R,4s,5S)-4-(4- (1H-pyrrolo[2,3- b]pyridin-4- yl)phenyl)-1,3,5- trimethylpiperidin- 4-ol | ¹H NMR (400 MHz, DMSO-d₆) δ 11.85 (s, 1H), 9.64 (s, 1H), 8.30 (d, J = 5.0 Hz, 1H), 7.84 (d, J = 8.1 Hz, 2H), 7.79 − 7.70 (m, 1H), 7.56 (t, J = 3.0 Hz, 1H), 7.38 − 7.28 (m, 1H), 7.24 (d, J = 5.0 Hz, 1H), 6.66 (dd, J = 3.5, 1.8 Hz, 1H), 5.26 (s, 1H), 3.31 (d, J = 11.8 Hz, 2H), 3.15 − 2.96 (m, 2H), 2.86 (d, J = 4.5 Hz, 3H), 2.36 (tt, J = 11.6, 9.3, 4.5 Hz, 2H), 0.60 (d, J = 6.8 Hz, 6H). | [M + H]⁺: 336.2 |
| 39 | 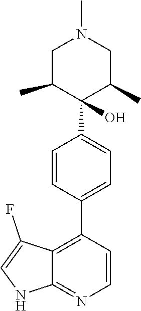 [SMILES: C[C@@H]1CN(C)C[C@H](C)[C@@]1(O)c1ccc(-c2ccnc3[nH]cc(F)c23)cc1] | (3R,4s,5S)-4-(4-(3- fluoro-1H- pyrrolo[2,3- b]pyridin-4- yl)phenyl)-1,3,5- trimethylpiperidin- 4-ol | ¹H NMR (400 MHz, DMSO-d₆) δ 11.66 (s, 1H), 9.62 (s, 1H), 8.32 (d, J = 4.9 Hz, 1H), 7.70 (d, J = 5.8 Hz, 3H), 7.53 (s, 1H), 7.32 − 7.22 (m, 1H), 7.18 (d, J = 5.0 Hz, 1H), 5.26 (s, 1H), 3.30 (d, J = 11.8 Hz, 2H), 3.02 (q, J = 11.6 Hz, 2H), 2.86 (d, J = 4.5 Hz, 3H), 2.41 − 2.31 (m, 2H), 0.59 (d, J = 6.8 Hz, 6H). | [M + H]⁺: 354.2 |
| 40 | 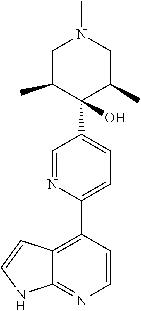 [SMILES: C[C@@H]1CN(C)C[C@H](C)[C@@]1(O)c1ccc(-c2ccnc3[nH]ccc23)nc1] | (3R,4s,5S)-4-(6- (1H-pyrrolo[2,3- b]pyridin-4- yl)pyridin-3-yl)- 1,3,5- trimethylpiperidin- 4-ol | ¹H NMR (400 MHz, DMSO-d₆) δ 11.83 (s, 1H), 9.52 − 9.68 (m, 2H), 8.34 (d, J = 5.1 Hz, 1H), 8.16 (d, J = 8.1 Hz, 1H), 7.65 (d, J = 5.1 Hz, 1H), 7.59 (s, 1H), 7.04 (d, J = 3.1 Hz, 1H), 5.48 (s, 1H), 3.33 (d, J = 11.9 Hz, 2H), 3.04 (q, J = 11.5 Hz, 2H), 2.87 (d, J = 4.4 Hz, 3H), 2.44 − 2.37 (m, 2H), 0.62 (d, J = 6.8 Hz, 6H). | [M + H]⁺: 337.2 |
| 41 | 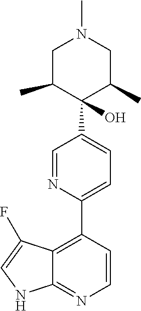 [SMILES: C[C@@H]1CN(C)C[C@H](C)[C@@]1(O)c1ccc(-c2ccnc3[nH]cc(F)c23)nc1] | (3R,4s,5S)-4-(6-(3- fluoro-1H- pyrrolo[2,3- b]pyridin-4- yl)pyridin-3-yl)- 1,3,5- trimethylpiperidin- 4-ol | ¹H NMR (400 MHz, DMSO-d₆) δ 11.72 (s, 1H), 9.69 (s, 1H), 8.36 (d, J = 4.9 Hz, 1H), 7.88 (dd, J = 7.9, 3.2 Hz, 1H), 7.57 (s, 1H), 7.47 (d, J = 5.0 Hz, 1H), 5.46 (s, 1H), 3.31 (d, J = 11.8 Hz, 2H), 3.04 (p, J = 12.8, 11.7 Hz, 2H), 2.85 (d, J = 4.5 Hz, 3H), 2.43 − 2.28 (m, 2H), 0.60 (d, J = 6.8 Hz, 6H). | [M + H]⁺: 355.2 |
| 42 | 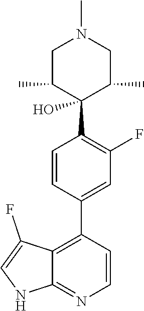 [SMILES: C[C@@H]1CN(C)C[C@H](C)[C@@]1(O)c1ccc(-c2ccnc3[nH]cc(F)c23)cc1F] | (3R,4s,5S)-4-(2- fluoro-4-(3-fluoro- 1H-pyrrolo[2,3- b]pyridin-4- yl)phenyl)-1,3,5- trimethylpiperidin- 4-ol | ¹H NMR (400 MHz, DMSO-d₆) δ 11.73 (s, 1H), 9.64 (s, 1H), 8.34 (d, J = 5.0 Hz, 1H), 7.74 (t, J = 8.3 Hz, 1H), 7.57 (s, 2H), 7.49 (d, J = 13.4 Hz, 1H), 7.26 − 7.14 (m, 1H), 5.50 (s, 1H), 3.27 (t, J = 13.3 Hz, 2H), 3.02 (p, J = 12.1 Hz, 2H), 2.83 (dd, J = 8.9, 4.5 Hz, 3H), 2.71 − 2.58 (m, 2H), 0.64 (d, J = 6.8 Hz, 6H). | [M + H]⁺: 372.2 |
| 43 | 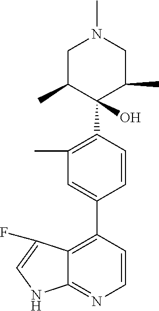 [SMILES: Cc1cc(-c2ccnc3[nH]cc(F)c23)ccc1[C@@]1(O)[C@H](C)CN(C)C[C@@H]1C] | (3R,4s,5S)-4-(4-(3- fluoro-1H- pyrrolo[2,3- b]pyridin-4-yl)-2- methylphenyl)- 1,3,5- trimethylpiperidin- 4-ol | ¹H NMR (400 MHz, DMSO-d₆) δ 11.63 (s, 1H), 9.65- 9.54 (m, 2H), 8.29 (dd, J = 4.9, 2.0 Hz, 1H), 7.78 (d, J = 8.3 Hz, 1H), 7.54 − 7.47 (m, 1H), 7.46 (s, 1H), 7.15 (dd, J = 4.9, 1.5 Hz, 1H), 5.22 (s, 1H), 3.25 (d, J = 11.4 Hz, 2H), 3.10 − 3.00 (m, 2H), 2.84 (d, J = 4.6 Hz, 4H), 2.79 − 2.69 (m, 2H), 2.66 (s, 1H), 2.52 (s, 2H), 0.70 (d, J = 6.9 Hz, 1H), 0.59 (d, J = 6.9 Hz, 5H). | [M + H]⁺: 368.2 |
| 44 | 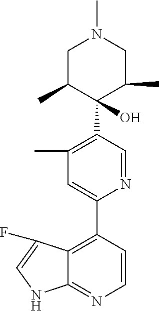 [SMILES: Cc1cc(-c2ccnc3[nH]cc(F)c23)ncc1[C@@]1(O)[C@H](C)CN(C)C[C@@H]1C] | (3R,4s,5S)-4-(6-(3- fluoro-1H- pyrrolo[2,3- b]pyridin-4-yl)-4- methylpyridin-3- yl)-1,3,5- trimethylpiperidin- 4-ol | DMSO-d₆) δ 11.72 (d, J = 3.3 Hz, 1H), 9.69 (s, 1H), 8.92 (s, 1H), 8.37 (d, J = 4.8 Hz, 1H), 7.65 (d, J = 3.2 Hz, 1H), 7.59 (d, J = 10.8 Hz, 1H), 7.47 (d, J = 5.0 Hz, 1H), 5.49 (d, J = 1.8 Hz, 1H), 3.28 (d, J = 11.6 Hz, 2H), 3.08 (q, J = 11.5 Hz, 2H), 2.87 (d, J = 4.5 Hz, 3H), 2.79 − 2.65 (m, 2H), 2.56 (s, 3H), 0.74 (d, J = 6.7 Hz, 1H), 0.63 (d, J = 6.7 Hz, 5H). | [M + H]⁺: 369.2 |
| 304 | 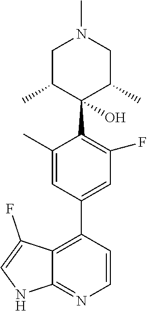 [SMILES: Cc1cc(-c2ccnc3[nH]cc(F)c23)cc(F)c1[C@@]1(O)[C@H](C)CN(C)C[C@@H]1C] | (3S,4s,5R)-4-(2- fluoro-4-(3-fluoro- 1H-pyrrolo[2,3- b]pyridin-4-yl)-6- methylphenyl)- 1,3,5- trimethylpiperidin- 4-ol | ¹H NMR (400 MHz, DMSO-d₆) δ 11.70 (s, 1H), 8.32 (d, J = 4.9 Hz, 1H), 7.55 (d, J = 2.8 Hz, 1H), 7.30 (d, J = 8.3 Hz, 2H), 7.21 (d, J = 4.9 Hz, 1H), 4.98 (s, 1H), 3.29 − 3.21 (m, 2H), 3.06 − 2.96 (m, 2H), 2.82 (d, J = 4.5 Hz, 3H), 2.81 − 2.72 (m, 2H), 2.70 (s, 3H), 0.77 (d, J = 6.8 Hz, 6H). | [M + H]⁺: 386.1 |

### Compound 45

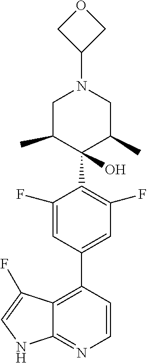 [SMILES: C[C@@H]1CN(C2COC2)C[C@H](C)[C@@]1(O)c1c(F)cc(-c2ccnc3[nH]cc(F)c23)cc1F]

### (3S,4s,5R)-4-(2,6-difluoro-4-(3-fluoro-1H-pyrrolo[2,3-b]pyridin-4-yl)phenyl)-3, S-dimethyl-1-(oxetan-3-yl)piperidin-4-ol

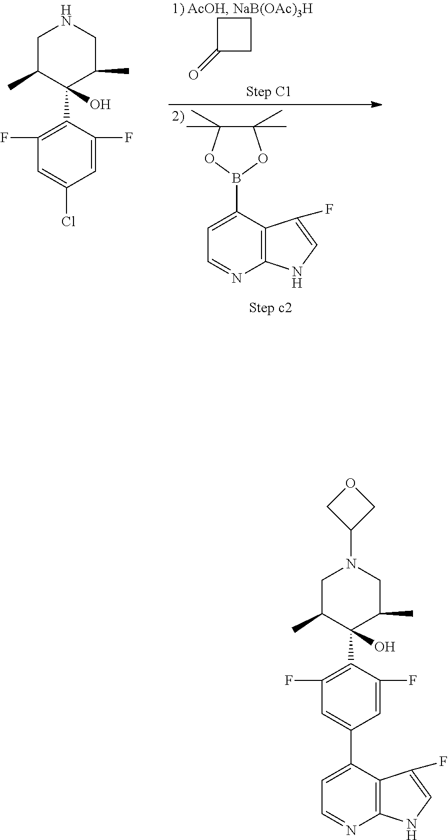

Step C1: To a solution of (3S,4s,5R)-4-(4-chloro-2,6-difluorophenyl)-3,5-dimethylpiperidin-4-ol (0.5 g, 0.181 mmol) in dichloromethane (3.0 ml) was added oxetan-3-one (0.020 g, 0.272 mmol), followed by acetic acid (0.005 g, 0.087 mmol). The reaction was stirred for 10 minutes at room temperature. Then sodium triacetoxyborohydride (0.100 g, 0.472 mmol) was added. The reaction was stirred at room temperature for a further 2 hours at room temperature. The reaction was then poured into a saturated solution of aqueous sodium bicarbonate and extracted with ethyl acetate (2×). The organic phases were partioned and combined, and the solvent removed by rotary evaporation to give the crude product which was used without further purification. LC-MS (m/z [M+H]⁺): 332.1

Step C2: To a 2 ml microwave vial was added potassium phosphate (0.100 g, 0.471 mmol, tribasic), followed by 3-fluoro-4-(4,4,5,5-tetramethyl-1,3,2-dioxaborolan-2-yl)-1H-pyrrolo[2,3-b]pyridine (0.050 g, 0.191 mmol) and Chloro(2-dicyclohexylphosphino-2′,4′,6′-triisopropyl-1,1′-biphenyl)[2-(2′-amino-1,1′-biphenyl)]palladium(II) (Xphos Pd G2, 15.0 mg, 0.019 mmol). The crude product from Step C1 was dissolved in a 1:1 mixture of tetrahydrofuran and water (2 ml total), and added to the 2 ml microwave vial. The vial was sealed and degassed with nitrogen bubbling for 5 minutes. It was then heated to 100 C for 30 minutes via microwave irradiation. The vial was cooled, and the solvents removed by rotary evaporation under reduced pressure to give the crude product. The crude product was diluted with 2 mL of methanol, was then filtered, and the collected solution was purified by reverse-phase HPLC to provide the title compound. ¹H NMR (400 MHz, DMSO-d₆) δ 11.76 (s, 1H), 8.41-8.21 (m, 1H), 7.59 (d, J=2.7 Hz, 1H), 7.33 (t, J=12.9 Hz, 2H), 7.26 (d, J=4.9 Hz, 1H), 4.64 (s, 1H), 4.54 (t, J=6.5 Hz, 2H), 4.47 (t, J=6.2 Hz, 2H), 3.45-3.37 (m, 1H), 2.50-2.27 (m, 4H), 2.03 (t, J=10.9 Hz, 2H), 0.76 (d, J=6.8 Hz, 6H). LC-MS (m/z [M+H]⁺): 432.2

### Compounds 46-54

Compounds 46-54 and 305 were prepared using an analogous procedure to that of Compound 45, and are shown in Table 9.

TABLE 9

| | | | | LCMS |
|---|---|---|---|---|
| Compound | Structure | IUPAC name | ¹H NMR | m/z |
| 46 | 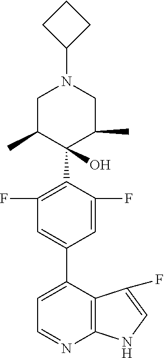 [SMILES: C[C@@H]1CN(C2CCC2)C[C@H](C)[C@@]1(O)c1c(F)cc(-c2ccnc3[nH]cc(F)c23)cc1F] | (3R,4s,5S)-1- cyclobutyl-4-(2,6- difluoro-4-(3- fluoro-1H- pyrrolo[2,3- b]pyridin-4- yl)phenyl)-3,5- dimethylpiperidin- 4-ol | ¹H NMR (400 MHz, DMSO-d₆) δ 11.76 (s, 1H), 8.34 (d, J = 4.9 Hz, 1H), 8.15 (s, 1H), 7.59 (s, 1H), 7.33 (t, J = 11.2 Hz, 2H), 7.26 (d, J = 4.9 Hz, 1H), 4.66 (s, 1H), 2.75 (d, J = 64.8 Hz, 1H), 2.58 − 2.52 (m, 2H), 2.48 − 2.31 (m, 2H), 2.05 (m, 4H), 1.92 − 1.78 (m, 2H), 1.64 (s, 2H), 0.77 (d, J = 6.8 Hz, 6H). | [M + H]⁺: 430.3 |
| 47 | 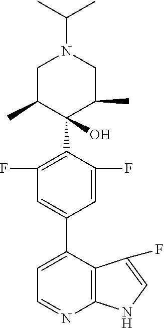 [SMILES: CC(C)N1C[C@@H](C)[C@](O)(c2c(F)cc(-c3ccnc4[nH]cc(F)c34)cc2F)[C@@H](C)C1] | (3R,4s,5S)-4-(2,6- difluoro-4-(3- fluoro-1H- pyrrolo[2,3- b]pyridin-4- yl)phenyl)-1- isopropyl-3,5- dimethylpiperidin- 4-ol | ¹H NMR (400 MHz, DMSO-d₆) δ 11.79 (s, 1H), 9.24 (s, 1H), 8.36 (d, J = 4.9 Hz, 1H), 7.61 (d, J = 2.9 Hz, 1H), 7.51 − 7.34 (m, 2H), 7.27 (d, J = 4.9 Hz, 1H), 5.51 (s, 1H), 3.19 (d, J = 11.6 Hz, 2H), 3.01 (q, J = 11.3 Hz, 2H), 2.71 (t, J = 13.2 Hz, 2H), 1.30 (d, J = 6.6 Hz, 6H), 0.86 (d, J = 6.8 Hz, 6H). | [M + H]⁺: 418.2 |
| 48 | 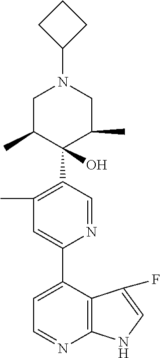 [SMILES: Cc1cc(-c2ccnc3[nH]cc(F)c23)ncc1[C@@]1(O)[C@H](C)CN(C2CCC2)C[C@@H]1C] | (3R,4s,5S)-1- cyclobutyl-4-(6-(3- fluoro-1H- pyrrolo[2,3- b]pyridin-4-yl)-4- methylpyridin-3- yl)-3,5- dimethylpiperidin- 4-ol | ¹H NMR (400 MHz, DMSO-d₆) δ 11.72 (s, 1H), 8.91 (s, 1H), 8.52 − 8.15 (m, 1H), 7.66 (d, J = 3.1 Hz, 1H), 7.58 (s, 1H), 7.47 (d, J = 5.0 Hz, 1H), 5.48 (s, 1H), 3.20 (d, J = 11.3 Hz, 2H), 2.84 (t, J = 11.0 Hz, 2H), 2.75 − 2.65 (m, 1H), 2.56 (s, 3H), 2.29 − 2.18 (m, 6H), 1.88 − 1.68 (m, 2H), 0.64 (d, J = 6.7 Hz, 6H). | [M + H]⁺: 409.3 |
| 49 | 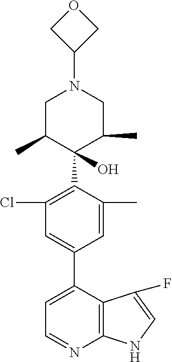 [SMILES: Cc1cc(-c2ccnc3[nH]cc(F)c23)cc(Cl)c1[C@@]1(O)[C@H](C)CN(C2COC2)C[C@@H]1C] | (3R,4s,5S)-4-(2- chloro-4-(3-fluoro- 1H-pyrrolo[2,3- b]pyridin-4-yl)-6- methylphenyl)-3,5- dimethyl-1-(oxetan- 3-yl)piperidin-4-ol | ¹H NMR (400 MHz, DMSO-d₆) δ 11.64 (s, 1H), 8.32 (d, J = 4.9 Hz, 1H), 8.01 (s, 1H), 7.62 − 7.42 (m, 2H), 7.16 (d, J = 4.9 Hz, 1H), 4.59 − 4.44 (m, 6H), 3.58 − 3.46 (m, 2H), 3.17 − 3.13 (m, 2H), 2.44 (s, 3H), 2.09 − 1.90 (m, 2H), 0.53 (d, J = 6.8 Hz, 6H). | [M + H]⁺: 444.2 |
| 50 | 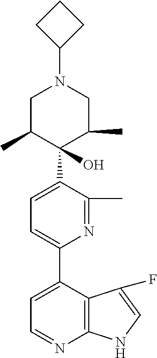 [SMILES: Cc1nc(-c2ccnc3[nH]cc(F)c23)ccc1[C@@]1(O)[C@H](C)CN(C2CCC2)C[C@@H]1C] | (3R,4s,5S)-1- cyclobutyl-4-(6-(3- fluoro-1H- pyrrolo[2,3- b]pyridin-4-yl)-2- methylpyridin-3- yl)-3,5- dimethylpiperidin- 4-ol | ¹H NMR (400 MHz, DMSO-d₆) δ 11.66 (s, 1H), 8.34 (t, J = 4.1 Hz, 1H), 8.20 − 8.12 (m, 1H), 7.82 − 7.58 (m, 1H), 7.55 (s, 1H), 7.48 (t, J = 3.5 Hz, 1H), 4.59 (s, 1H), 2.86 (s, 1H), 2.73 (s, 2H), 2.69 − 2.52 (m, 2H), 2.15 − 1.94 (m, 4H), 1.86 (dt, J = 18.1, 9.3 Hz, 2H), 1.70 − 1.52 (m, 2H), 0.64 (d, J = 6.6 Hz, 2H), 0.54 (d, J = 6.6 Hz, 4H). | [M + H]⁺: 409.3 |
| 51 | 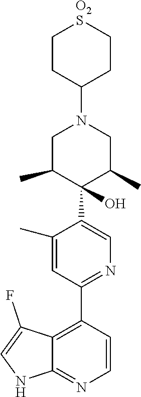 [SMILES: Cc1cc(-c2ccnc3[nH]cc(F)c23)ncc1[C@@]1(O)[C@H](C)CN(C2CCS(=O)(=O)CC2)C[C@@H]1C] | 4-((3R,4s,5S)-4-(6- (3-fluoro-1H- pyrrolo[2,3- b]pyridin-4-yl)-4- methylpyridin-3- yl)-4-hydroxy-3,5- dimethylpiperidin- 1-yl)tetrahydro-2H- thiopyran 1,1- dioxide | ¹H NMR (400 MHz, DMSO-d₆) δ 11.73 (s, 1H), 8.66 (d, J = 5.0 Hz, 1H), 8.48 − 8.22 (m, 2H), 8.13 (s, 1H), 7.75 − 7.58 (m, 2H), 7.45 (d, J = 4.9 Hz, 1H), 7.31 (d, J = 5.1 Hz, 1H), 3.11 − 2.95 (m, 7H), 2.90 (s, 3H), 1.99 − 1.86 (m, 5H), 1.61 − 1.48 (m, 2H), 1.01 (d, J = 6.6 Hz, 6H). | [M + H]⁺: 487.3 |
| 52 | 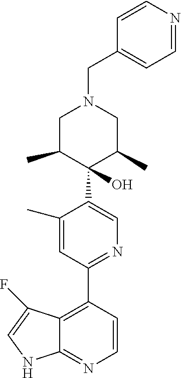 [SMILES: Cc1cc(-c2ccnc3[nH]cc(F)c23)ncc1[C@@]1(O)[C@H](C)CN(Cc2ccncc2)C[C@@H]1C] | (3R,4s,5S)-4-(6-(3- fluoro-1H- pyrrolo[2,3- b]pyridin-4-yl)-4- methylpyridin-3- yl)-3,5-dimethyl-1- (pyridin-4- ylmethyl)piperidin- 4-ol | | [M + H]⁺: 446.3 |
| 53 | 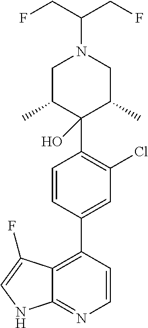 [SMILES: C[C@@H]1CN(C(CF)CF)C[C@H](C)C1(O)c1ccc(-c2ccnc3[nH]cc(F)c23)cc1Cl] | (3S,5R)-4-(2- chloro-4-(3-fluoro- 1H-pyrrolo[2,3- b]pyridin-4- yl)phenyl)-1-(1,3- difluoropropan-2- yl)-3,5- dimethylpiperidin- 4-ol | ¹H NMR (400 MHz, DMSO-d₆) δ 11.70 (s, 1H), 8.32 (d, J = 5.0 Hz, 1H), 7.95 (d, J = 8.2 Hz, 1H), 7.78 − 7.63 (m, 2H), 7.56 (s, 1H), 7.23 (d, J = 4.8 Hz, 1H), 4.75 − 4.55 (m, 4H), 3.19 − 3.03 (m, 1H), 3.03 − 2.91 (m, 2H), 2.74 − 2.65 (m, 2H), 2.64 − 2.56 (m, 2H), 0.53 (d, J = 6.9 Hz, 6H). | [M + H]⁺: 452.2 |
| 54 | 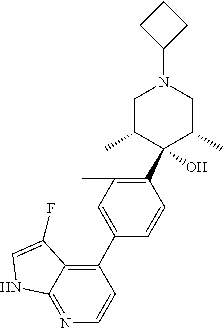 [SMILES: Cc1cc(-c2ccnc3[nH]cc(F)c23)ccc1[C@@]1(O)[C@H](C)CN(C2CCC2)C[C@@H]1C] | (3R,4s,5S)-1- cyclobutyl-4-(4-(3- fluoro-1H- pyrrolo[2,3- b]pyridin-4-yl)-2- methylphenyl)-3,5- dimethylpiperidin- 4-ol | ¹H NMR (400 MHz, DMSO-d₆) δ 11.60 (s, 1H), 8.27 (t, J = 4.3 Hz, 1H), 8.15 (s, 1H), 7.80 (d, J = 8.2 Hz, 1H), 7.52 − 7.39 (m, 3H), 7.35 − 7.28 (m, 1H), 7.15 (t, J = 4.7 Hz, 1H), 4.52 (s, 1H), 3.01 − 2.81 (m, 1H), 2.64 (s, 1H), 2.52 (s, 2H), 2.2 − 2.11 (m, 2H), 2.09 − 1.99 (m, 2H), 1.90 (q, J = 9.9 Hz, 1H), 1.71 − 1.60 (m, 2H), 0.63 (d, J = 6.5 Hz, 2H), 0.53 (d, J = 6.7 Hz, 4H). | [M + H]⁺: 408.2 |
| 305 | 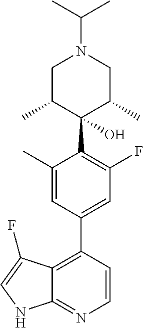 [SMILES: Cc1cc(-c2ccnc3[nH]cc(F)c23)cc(F)c1[C@@]1(O)[C@H](C)CN(C(C)C)C[C@@H]1C] | (3S,4s,5R)-4-(2- fluoro-4-(3-fluoro- 1H-pyrrolo[2,3- b]pyridin-4-yl)-6- methylphenyl)-1- isopropyl-3,5- dimethylpiperidin- 4-ol | ¹H NMR (400 MHz, DMSO-d₆) δ 11.71 (s, 1H), 9.26 (s, 1H), 8.32 (d, J = 4.9 Hz, 1H), 7.56 (t, J = 2.4 Hz, 1H), 7.33 (d, J = 8.5 Hz, 2H), 7.22 (d, J = 4.9 Hz, 1H), 5.02 (s, 1H), 3.56 − 3.54 (m, 2H), 3.32 − 3.14 (m, 2H), 3.02 (q, J = 11.7 Hz, 2H), 2.87 − 2.75 (m, 2H), 2.71 (s, 3H), 1.31 (d, J = 6.6 Hz, 6H), 0.80 (d, J = 6.8 Hz, 6H). | [M + H]⁺: 414.1 |

### Compound 55

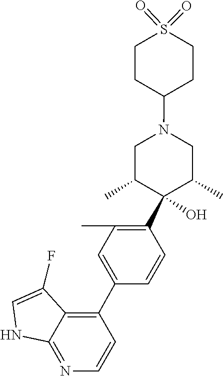 [SMILES: Cc1cc(-c2ccnc3[nH]cc(F)c23)ccc1[C@@]1(O)[C@H](C)CN(C2CCS(=O)(=O)CC2)C[C@@H]1C]

### 4-((3R,4s, 5S)-4-(4-(3-fluoro-1H-pyrrolo[2,3-b]pyridin-4-yl)-2-methylphenyl)-4-hydroxy-3,5S-dimethylpiperidin-1-yl)tetrahydro-2H-thiopyran 1,1-dioxide

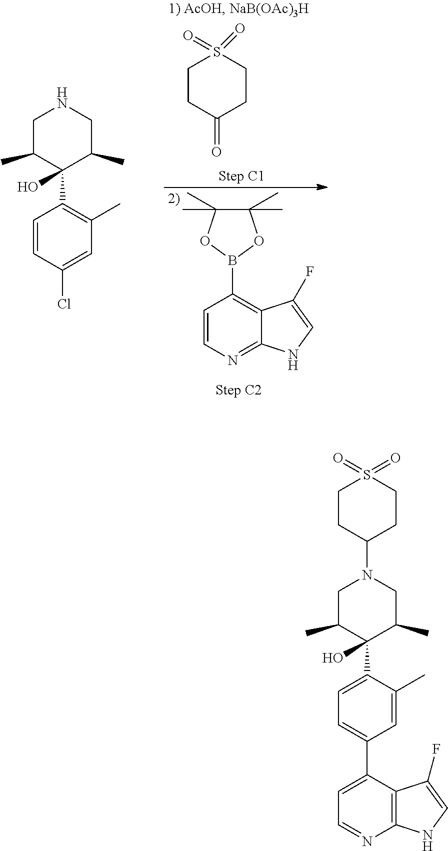

Step C1: To a solution (3R,4s,5S)-4-(4-chloro-2-methylphenyl)-3,5-dimethylpiperidin-4-ol (0.220 g, 0.867 mmol) in tetrahydrofuran (14.0 ml) was added tetrahydro-4H-thiopyran-4-one 1,1-dioxide (0.0.257 g, 1.73 mmol), followed by acetic acid (0.010 g, 0.175 mmol). The reaction was stirred for 30 minutes at room temperature. Then sodium triacetoxyborohydride (0.551 g, 2.60 mmol) was added. The reaction was then placed in a sealed vial and heated to 80° C. for 1 hour. The reaction was cooled, then poured into a saturated solution of aqueous sodium bicarbonate and extracted with ethyl acetate (2×). The organic phases were partioned and combined, and the solvent removed by rotary evaporation to give the crude product which was used without further purification. LC-MS (m/z [M+H]⁺): 386.1

Step C2: To a 20 ml microwave vial was added potassium phosphate (0.500 g, 2.35 mmol, tribasic), followed by 3-fluoro-4-(4,4,5,5-tetramethyl-1,3,2-dioxaborolan-2-yl)-1H-pyrrolo[2,3-b]pyridine (0.271 g, 0.829 mmol) and Chloro(2-dicyclohexylphosphino-2′,4′,6′-triisopropyl-1,1′-biphenyl)[2-(2′-amino-1,1′-biphenyl)]palladium(II) (Xphos Pd G2, 50.0 mg, 0.063 mmol). The crude product from Step C1 was dissolved in a 1:1 mixture of tetrahydrofuran and water (10 ml total), and added to the 20 ml microwave vial. The vial was sealed and degassed with nitrogen bubbling for 5 minutes. It was then heated to 100 C for 40 minutes via microwave irradiation. The vial was cooled, and the reaction mixture was extracted with ethyl acetate (2×). The organic solvent was then removed by rotary evaporation to give the crude product. The crude product was purified by reverse-phase chromatography (5% to 50% acetonitrile in water, with 0.1% trifluoroacetic acid) to provide the title compound.

¹H NMR (400 MHz, DMSO-d₆) δ 11.61 (s, 1H), 8.30 (dd, J=4.9, 2.4 Hz, 1H), 7.84 (d, J=8.3 Hz, 1H), 7.54-7.49 (m, 1H), 7.49-7.33 (m, 2H), 7.17 (t, J=5.0 Hz, 1H), 4.36 (s, 1H), 4.09 (d, J=5.8 Hz, 1H), 3.18 (d, J=4.3 Hz, 2H), 3.16-3.07 (m, 3H), 2.67 (s, 1H), 2.55 (s, 2H), 2.49-2.30 (m, 2H), 2.23-2.13 (m, 3H), 2.09-2.00 (m, 4H), 0.66 (d, J=6.8 Hz, 2H), 0.55 (d, J=6.1 Hz, 4H). LC-MS (m/z [M+H]⁺): 486.2

### Compounds 56-57

Compounds 56-57 and 306-309 were prepared using an analogous procedure to that of Compound 55, and are shown in Table 10.

TABLE 10

| | | | | LCMS |
|---|---|---|---|---|
| Compound | Structure | IUPAC name | ¹H NMR | m/z |
| 56 | 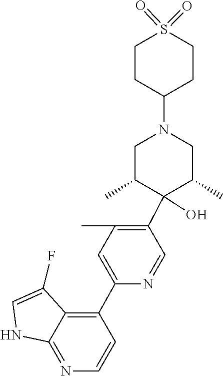 | 4-((3R,5S)-4-(6-(3- fluoro-1H- pyrrolo[2,3- b]pyridin-4-yl)-4- methylpyridin-3- yl)-4-hydroxy-3,5- dimethylpiperidin- 1-yl)tetrahydro-2H- thiopyran 1,1- dioxide | ¹H NMR (400 MHz, DMSO-d₆) δ 11.74 (s, 1H), 8.66 (d, J = 5.0 Hz, 1H), 8.57-8.23 (m, 2H), 8.14 (s, 1H), 7.66 (s, 1H), 7.60 (t, J = 2.5 Hz, 1H), 7.45 (d, J = 4.9 Hz, 1H), 7.31 (d, J = 5.1 Hz, 1H), 3.12-2.96 (m, 7H), 2.91 (s, 3H), 2.38-2.22 (m, 2H), 1.99-1.87 (m, 4H), 1.60-1.50 (m, 2H), 1.02 (d, J = 6.6 Hz, 6H). | [M + H]⁺: 427.3 |
| 57 | 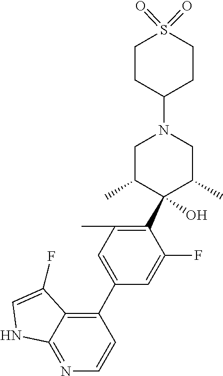 [SMILES: Cc1cc(-c2ccnc3[nH]cc(F)c23)cc(F)c1[C@@]1(O)[C@H](C)CN(C2CCS(=O)(=O)CC2)C[C@@H]1C] | 4-((3R,4s,5S)-4-(2- fluoro-4-(3-fluoro- 1H-pyrrolo[2,3- b]pyridin-4-yl)-6- methylphenyl)-4- hydroxy-3,5- dimethylpiperidin- 1-yl)tetrahydro-2H- thiopyran 1,1- dioxide | ¹H NMR (400 MHz, DMSO-d₆) δ 11.71 (s, 1H), 8.32 (d, J = 4.8 Hz, 1H), 7.56 (s, 1H), 7.35- 7.28 (m, 2H), 7.22 (d, J = 5.0 Hz, 1H), 5.02 (d, J = 21.6 Hz, 1H), 3.62-3.53 (m, 1H), 3.48-3.44 (m, 1H), 3.32-3.22 (m, 5H), 3.20-3.05 (m, 1H), 2.89- 2.79 (m, 1H), 2.71 (s, 3H), 2.47-2.41 (m, 1H), 2.27-2.17 (m, 1H), 1.80- 1.70 (m, 1H), 1.53- 1.43 (m, 1H), 1.31-1.20 (m, 1H), 0.80 (d, J = 6.8 Hz, 6H). | [M + H]⁺: 504.2 |
| 306 | 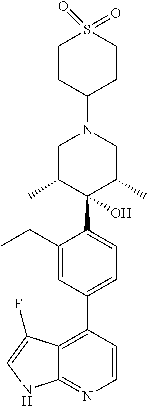 [SMILES: CCc1cc(-c2ccnc3[nH]cc(F)c23)ccc1[C@@]1(O)[C@H](C)CN(C2CCS(=O)(=O)CC2)C[C@@H]1C] | 4-((3S,4s,5R)-4-(2- ethyl-4-(3-fluoro- 1H-pyrrolo[2,3- b]pyridin-4- yl)phenyl)-4- hydroxy-3,5- dimethylpiperidin- 1-yl)tetrahydro-2H- thiopyran 1,1- dioxide | ¹H NMR (400 MHz, DMSO-d₆) δ 11.59 (s, 1H), 8.28 (dd, J = 5.1, 2.2 Hz, 1H), 7.87-7.73 (m, 1H), 7.57-7.25 (m, 3H), 7.16 (td, J = 5.3, 2.9 Hz, 1H), 4.31 (s, 1H), 3.20-3.04 (m, 6H), 2.91- 2.82 (m, 1H), 2.72- 2.63 (m, 1H), 2.44-2.36 (m, 4H), 2.07-1.95 (m, 3H), 1.26-1.13 (m, 3H), 0.65-0.48 (m, 6H). | [M + H]⁺: 500.1 |
| 307 | 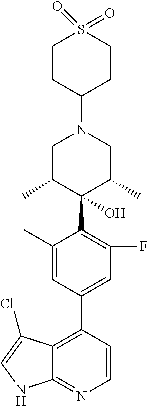 [SMILES: Cc1cc(-c2ccnc3[nH]cc(Cl)c23)cc(F)c1[C@@]1(O)[C@H](C)CN(C2CCS(=O)(=O)CC2)C[C@@H]1C] | 4-((3S,4s,5R)-4-(4- (3-chloro-1H- pyrrolo[2,3- b]pyridin-4-yl)-2- fluoro-6- methylphenyl)-4- hydroxy-3,5- dimethylpiperidin- 1-yl)tetrahydro-2H- thiopyran 1,1- dioxide | ¹H NMR (400 MHz, DMSO-d₆) δ 12.18 (s, 1H), 8.31 (d, J = 4.8 Hz, 1H), 7.70 (d, J = 2.7 Hz, 1H), 7.15 (d, J = 14.4 Hz, 2H), 7.08 (d, J = 4.8 Hz, 1H), 5.01 (s, 1H), 3.61-3.49 (m, 4H), 3.32- 3.20 (m, 4H), 3.17- 3.00 (m, 2H), 2.67 (s, 3H), 2.99-2.77 (m, 2H), 2.44-2.40 (m, 1H), 2.26- 2.11 (m, 2H), 0.78 (d, J = 6.8 Hz, 6H). | [M + H]⁺: 520.0 |
| 308 | 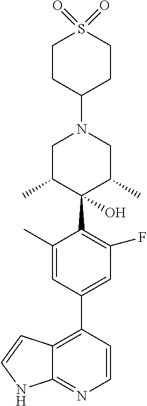 [SMILES: Cc1cc(-c2ccnc3[nH]ccc23)cc(F)c1[C@@]1(O)[C@H](C)CN(C2CCS(=O)(=O)CC2)C[C@@H]1C] | 4-((3S,4s,5R)-4-(2- fluoro-6-methyl-4- (1H-pyrrolo[2,3- b]pyridin-4- yl)phenyl)-4- hydroxy-3,5- dimethylpiperidin- 1-yl)tetrahydro-2H- thiopyran 1,1- dioxide | ¹H NMR (400 MHz, DMSO-d₆) δ 11.85 (s, 1H), 8.28 (d, J = 5.0 Hz, 1H), 7.57 (t, J = 3.1 Hz, 1H), 7.42 (d, J = 14.5 Hz, 2H), 7.24 (d, J = 5.0 Hz, 1H), 6.66-6.63 (m, 1H), 5.04 (s, 1H), 3.36- 3.22 (m, 4H), 3.21-3.03 (m, 2H), 3.02-2.76 (m, 4H), 2.73 (s, 3H), 2.46- 2.40 (m, 1H), 2.29-2.16 (m, 1H), 1.30-1.22 (m, 2H), 0.79 (dd, J = 6.8, 3.3 Hz, 6H). | [M + H]⁺: 486.2 |
| 309 | 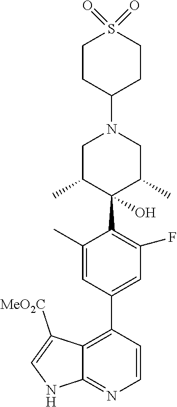 [SMILES: COC(=O)c1c[nH]c2nccc(-c3cc(C)c([C@@]4(O)[C@H](C)CN(C5CCS(=O)(=O)CC5)C[C@@H]4C)c(F)c3)c12] | methyl 4-(4- ((3S,4s,5R)-1-(1,1- dioxidotetrahydro- 2H-thiopyran-4-yl)- 4-hydroxy-3,5- dimethylpiperidin- 4-yl)-3-fluoro-5- methylphenyl)-1H- pyrrolo[2,3- b]pyridine-3- carboxylate | ¹H NMR (400 MHz, DMSO-d₆) δ 12.59 (s, 1H), 8.34 (d, J = 5.0 Hz, 1H), 8.20 (d, J = 2.3 Hz, 1H), 7.17 (d, J = 4.9 Hz, 1H), 6.97 (d, J = 15.4 Hz, 2H), 4.12 (s, 1H), 3.33 (d, J = 1.4 Hz, 3H), 3.19-3.03 (m, 4H), 2.71-2.65 (m, 2H), 2.65 (s, 3H), 2.59-2.52 (m, 1H) 2.45 (t, J = 3.1 Hz, 4H), 2.07-1.98 (m, 4H), 0.73 (d, J = 6.7 Hz, 6H). | [M + H]⁺: 544.3 |

### Synthesis of Compounds of General Formula A4

Compounds of general formula A4 can be prepared as illustrated in Scheme 4 below. Intermediates 1b are synthesized as per Scheme 1, and intermediates 2c are synthesized as per Scheme 2.

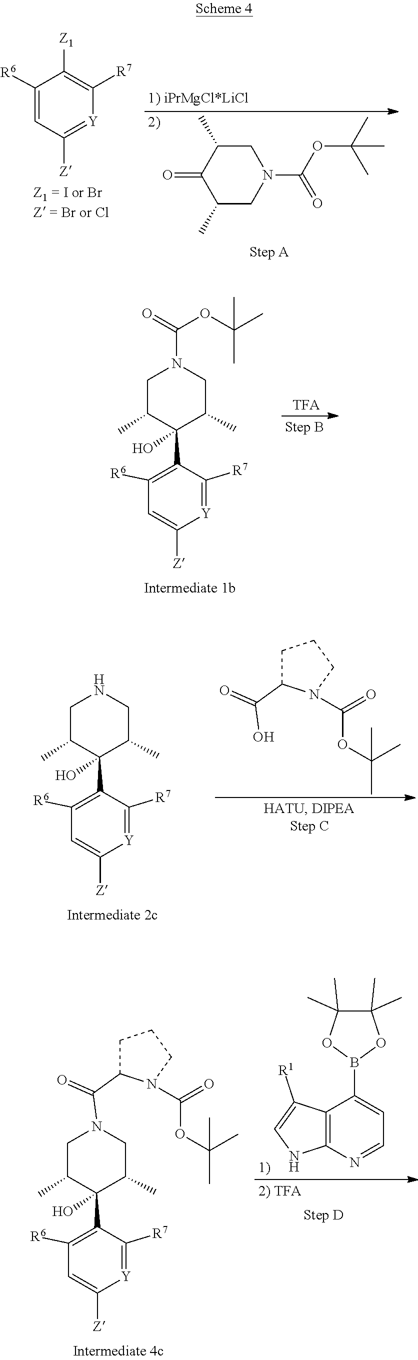

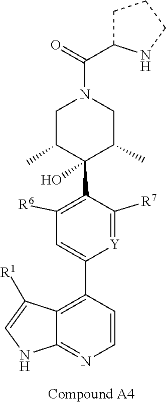

### Synthesis of Intermediates and Compounds of Scheme 4

### Intermediate 4c-1 (Scheme 4): tert-butyl (2-((3S,4s,5R)-4-(4-chloro-2,6-difluorophenyl)-4-hydroxy-3,5-dimethylpiperidin-1-yl)-2-oxoethyl)carbamate

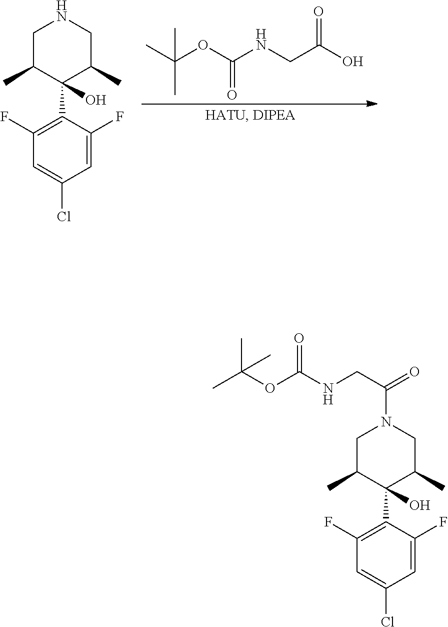

Step C: To a solution of (3S,4s,5R)-4-(4-chloro-2,6-difluorophenyl)-3,5-dimethylpiperidin-4-ol (0.05 g, 0.181 mmol) in dichloromethane (5.0 mL) was added (tert-butoxycarbonyl)glycine (0.032 g, 0.181 mmol), followed by 1-[bis(dimethylamino)methylene]-1H-1,2,3-triazolo[4,5-b]pyridinium 3-oxide hexafluorophosphate (0.069 g, 0.181 mmol, HATU) then diisopropylethyl amine (0.074 g, 0.573 mmol). The reaction was stirred at room temperature for 2 hours. The reaction was then poured into saturated aqueous sodium bicarbonate, and then extracted into dichloromethane. The organic layer was partitioned, and the solvent removed by rotary evaporation. Purification by silica gel chromatography gave the title product. LC-MS (m/z [M+H]⁺): 433.1

### Intermediates 4c-2 to 4c-14 (Scheme 4)

Intermediates 4c-2 to 4c-14 were prepared using an analogous procedure to that of Intermediate 4c-1 and are shown in Table 11.

TABLE 11

| Compound | Structure | IUPAC name | LCMS m/z |
|---|---|---|---|
| 4c-2 | 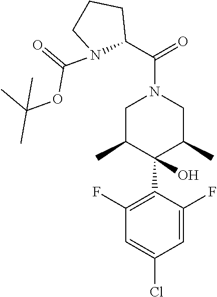 [SMILES: C[C@@H]1CN(C(=O)[C@H]2CCCN2C(=O)OC(C)(C)C)C[C@H](C)[C@@]1(O)c1c(F)cc(Cl)cc1F] | tert-butyl (R)-2-((3R,4S,5S)-4-(4- chloro-2,6-difluorophenyl)-3,4,5- trimethylpiperidine-1- carbonyl)pyrrolidine-1-carboxylate | [M + H]⁺: 473.2 |
| 4c-3 | 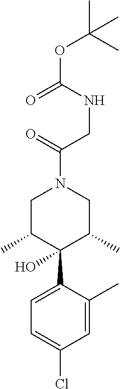 [SMILES: Cc1cc(Cl)ccc1[C@@]1(O)[C@H](C)CN(C(=O)CNC(=O)OC(C)(C)C)C[C@@H]1C] | tert-butyl (2-((3R,4s,5S)-4-(4- chloro-2-methylphenyl)-4-hydroxy- 3,5-dimethylpiperidin-1-yl)-2- oxoethyl)carbamate | [M + H]⁺: 411.2 |
| 4c-4 | 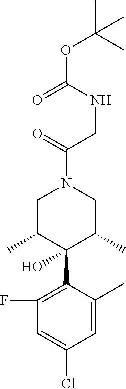 [SMILES: Cc1cc(Cl)cc(F)c1[C@@]1(O)[C@H](C)CN(C(=O)CNC(=O)OC(C)(C)C)C[C@@H]1C] | tert-butyl (2-((3R,4s,5S)-4-(4- chloro-2-fluoro-6-methylphenyl)-4- hydroxy-3,5-dimethylpiperidin-1- yl)-2-oxoethyl)carbamate | [M + H]⁺: 429.2 |
| 4c-5 | 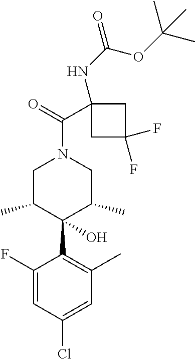 [SMILES: Cc1cc(Cl)cc(F)c1[C@@]1(O)[C@H](C)CN(C(=O)C2(NC(=O)OC(C)(C)C)CC(F)(F)C2)C[C@@H]1C] | tert-butyl (1-((3S,4s,5R)-4-(4- chloro-2-fluoro-6-methylphenyl)-4- hydroxy-3,5-dimethylpiperidine-1- carbonyl)-3,3- difluorocyclobutyl)carbamate | [M + H]⁺: 505.2 |
| 4c-6 | 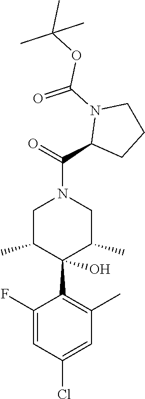 [SMILES: Cc1cc(Cl)cc(F)c1[C@@]1(O)[C@H](C)CN(C(=O)[C@@H]2CCCN2C(=O)OC(C)(C)C)C[C@@H]1C] | tert-butyl (S)-2-((3S,4R,5R)-4-(4- chloro-2-fluoro-6-methylphenyl)-4- hydroxy-3,5-dimethylpiperidine-1- carbonyl)pyrrolidine-1-carboxylate | [M + H]⁺: 469.3 |
| 4c-7 | 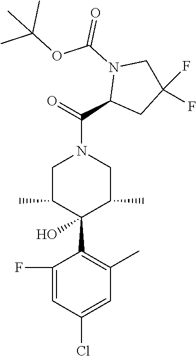 [SMILES: Cc1cc(Cl)cc(F)c1[C@@]1(O)[C@H](C)CN(C(=O)[C@@H]2CC(F)(F)CN2C(=O)OC(C)(C)C)C[C@@H]1C] | tert-butyl (S)-2-((3S,4R,5R)-4-(4- chloro-2-fluoro-6-methylphenyl)-4- hydroxy-3,5-dimethylpiperidine-1- carbonyl)-4,4-difluoropyrrolidine-1- carboxylate | [M + H]⁺: 505.3 |
| 4c-8 | 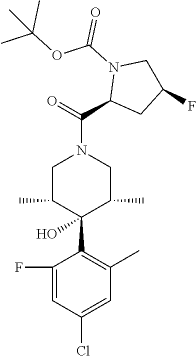 [SMILES: Cc1cc(Cl)cc(F)c1[C@@]1(O)[C@H](C)CN(C(=O)[C@@H]2C[C@H](F)CN2C(=O)OC(C)(C)C)C[C@@H]1C] | tert-butyl (2S,4S)-2-((3S,4R,5R)-4- (4-chloro-2-fluoro-6-methylphenyl)- 4-hydroxy-3,5-dimethylpiperidine- 1-carbonyl)-4-fluoropyrrolidine-1- carboxylate | [M + H]⁺: 487.3 |
| 4c-9 | 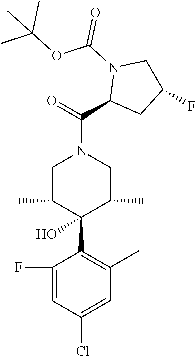 [SMILES: Cc1cc(Cl)cc(F)c1[C@@]1(O)[C@H](C)CN(C(=O)[C@@H]2C[C@@H](F)CN2C(=O)OC(C)(C)C)C[C@@H]1C] | tert-butyl (2S,4R)-2-((3S,4R,5R)-4- (4-chloro-2-fluoro-6-methylphenyl)- 4-hydroxy-3,5-dimethylpiperidine- 1-carbonyl)-4-fluoropyrrolidine-1- carboxylate | [M + H]⁺: 487.2 |
| 4c-10 | 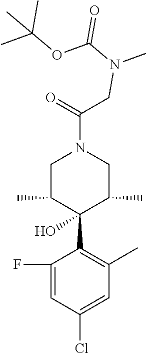 [SMILES: Cc1cc(Cl)cc(F)c1[C@@]1(O)[C@H](C)CN(C(=O)CN(C)C(=O)OC(C)(C)C)C[C@@H]1C] | tert-butyl (2-((3S,4s,5R)-4-(4- chloro-2-fluoro-6-methylphenyl)-4- hydroxy-3,5-dimethylpiperidin-1- yl)-2-oxoethyl)(methyl)carbamate | [M + H]⁺: 443.2 |
| 4c-11 | 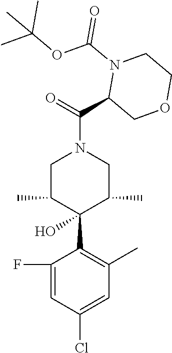 [SMILES: Cc1cc(Cl)cc(F)c1[C@@]1(O)[C@H](C)CN(C(=O)[C@@H]2COCCN2C(=O)OC(C)(C)C)C[C@@H]1C] | tert-butyl (S)-3-((3S,4R,5R)-4-(4- chloro-2-fluoro-6-methylphenyl)-4- hydroxy-3,5-dimethylpiperidine-1- carbonyl)morpholine-4-carboxylate | [M + H]⁺: 485.2 |
| 4c-12 | 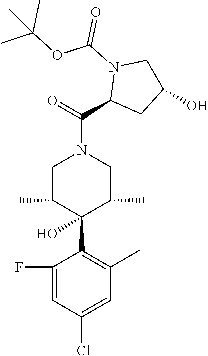 [SMILES: Cc1cc(Cl)cc(F)c1[C@@]1(O)[C@H](C)CN(C(=O)[C@@H]2C[C@@H](O)CN2C(=O)OC(C)(C)C)C[C@@H]1C] | tert-butyl (2S,4R)-2-((3S,4R,5R)-4- (4-chloro-2-fluoro-6-methylphenyl)- 4-hydroxy-3,5-dimethylpiperidine- 1-carbonyl)-4-hydroxypyrrolidine-1- carboxylate | [M + H]⁺: 484.2 |
| 4c-13 | 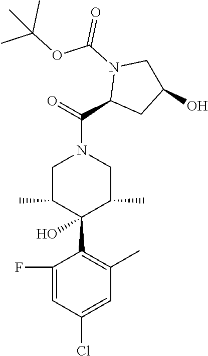 [SMILES: Cc1cc(Cl)cc(F)c1[C@@]1(O)[C@H](C)CN(C(=O)[C@@H]2C[C@H](O)CN2C(=O)OC(C)(C)C)C[C@@H]1C] | tert-butyl (2S,4S)-2-((3S,4R,5R)-4- (4-chloro-2-fluoro-6-methylphenyl)- 4-hydroxy-3,5-dimethylpiperidine- 1-carbonyl)-4-hydroxypyrrolidine-1- carboxylate | [M + H]⁺: 484.2 |
| 4c-14 | 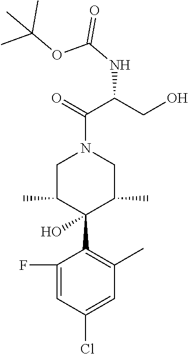 [SMILES: Cc1cc(Cl)cc(F)c1[C@@]1(O)[C@H](C)CN(C(=O)[C@@H](CO)NC(=O)OC(C)(C)C)C[C@@H]1C] | tert-butyl ((R)-1-((3S,4S,5R)-4-(4- chloro-2-fluoro-6-methylphenyl)-4- hydroxy-3,5-dimethylpiperidin-1- yl)-3-hydroxy-1-oxopropan-2- yl)carbamate | [M + H]⁺: 459.2 |

### Compound 58

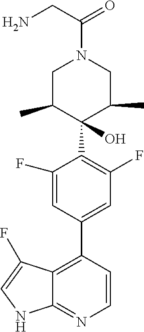 [SMILES: C[C@@H]1CN(C(=O)CN)C[C@H](C)[C@@]1(O)c1c(F)cc(-c2ccnc3[nH]cc(F)c23)cc1F]

### tert-butyl (2-((3S,4s,5R)-4-(4-chloro-2,6-difluorophenyl)-4-hydroxy-3,5-dimethylpiperidin-1-yl)-2-oxoethyl)carbamate

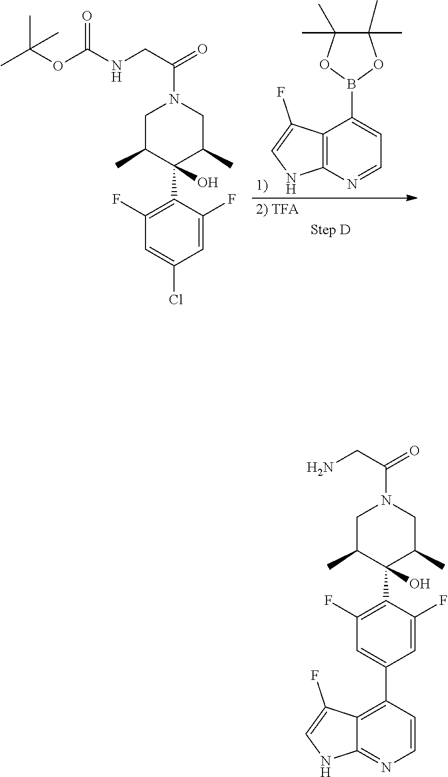

Step D1: A microwave vial was charged with tert-butyl (2-((3S,4s,5R)-4-(4-chloro-2,6-difluorophenyl)-4-hydroxy-3,5-dimethylpiperidin-1-yl)-2-oxoethyl)carbamate (0.047 g, 0.109 mmol), 3-fluoro-4-(4,4,5,5-tetramethyl-1,3,2-dioxaborolan-2-yl)-1H-pyrrolo[2,3-b]pyridine (0.05 g, 0.191 mmol), potassium phosphate (tribasic, K₃PO₄, 0.100 g, 0.471 mmol), and Chloro(2-dicyclohexylphosphino-2′,4′,6′-triisopropyl-1,1′-biphenyl)[2-(2′-amino-1,1′-biphenyl)]palladium(II) (Xphos Pd G2, 15.0 mg, 0.019 mmol). The vial was capped. Then, tetrahydrofuran (1.0 mL) was added, followed by water (1.0 ml). The content of the reaction vial was purged with nitrogen via bubbling through the solvent. The reaction was then heated in the microwave reactor at 100° C. for 45 min. The reaction was cooled to room temperature and poured into an aqueous solution of sodium bicarbonate. The mixture was extracted with ethyl acetate, the organic phases were partitioned and the organic solvent removed by rotary evaporation. Purification by reverse phase chromatography (5% to 100% acetonitrile in water with 0.1% trifluoroacetic acid) gave the semi purified product. The semi purified product was treated with aqueous sodium bicarbonate and extracted into ethyl acetate. The organic phases were partitioned, and the solvent removed by rotary evaporation to give the semi purified product as a free base. LC-MS (m/z [M+H]_): 533.2

Step D2: The semipurified product from step D1 was dissolved in dichloromethane (1 ml) and treated with trifluoroacetic acid (1.48 g, 1.0 mL, 13.0 mmol). The reaction was stirred for 30 minutes at room temperature. The solvent and trifluoroacetic acid were then removed by rotary evaporation. The crude product was treated with an aqueous solution of sodium bicarbonate, extracted into ethyl acetate, the organic phases were taken, and the solvent removed by rotary evaporation to give the crude product. The crude product was dissolved in methanol (1.0 mL) and purified by reverse-phase HPLC to provide the title compound. ¹H NMR (400 MHz, DMSO-d₆) δ 11.78 (s, 1H), 8.34 (d, J=4.9 Hz, 1H), 8.00 (s, 3H), 7.60 (s, 1H), 7.36 (d, J=13.1 Hz, 2H), 7.26 (d, J=5.0 Hz, 1H), 5.13 (s, 1H), 4.20 (d, J=12.7 Hz, 1H), 4.08 (dd, J=16.6, 5.9 Hz, 1H), 3.85 (dd, J=16.3, 5.7 Hz, 1H), 3.42 (d, J=12.9 Hz, 1H), 3.15 (t, J=12.4 Hz, 1H), 2.79 (t, J=12.3 Hz, 1H), 2.44-2.35 (m, 1H), 2.34-2.22 (m, 1H), 0.83 (d, J=6.7 Hz, 6H). LC-MS (m/z [M+H]⁺): 433.2

### Compounds 59-70

Compounds 59-70 and 310-313 were prepared using an analogous procedure to that of Compound 58 and are shown in Table 12.

TABLE 12

| | | | | LCMS |
|---|---|---|---|---|
| Compound | Structure | IUPAC name | ¹H NMR | m/z |
| 59 | 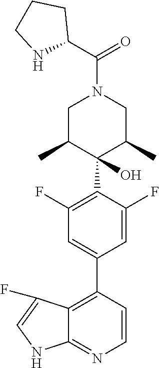 [SMILES: C[C@@H]1CN(C(=O)[C@H]2CCCN2)C[C@H](C)[C@@]1(O)c1c(F)cc(-c2ccnc3[nH]cc(F)c23)cc1F] | 4-(4-((3S,4R,5R)- 1-(L-prolyl)-4- hydroxy-3,5- dimethylpiperidin- 4-yl)-3,5- difluorophenyl)-3- fluoro-1H- pyrrolo[2,3- b]pyridine | ¹H NMR (400 MHz, DMSO-d₆) δ 11.75 (s, 1H), 8.32 (d, J = 4.9 Hz, 1H), 7.57 (s, 1H), 7.41-7.29 (m, 2H), 7.24 (dd, J = 5.0, 1.9 Hz, 1H), 5.03 (d, J = 35.2 Hz, 1H), 4.36-4.04 (m, 2H), 3.62 (d, J = 13.2 Hz, 1H), 3.13 (d, J = 19.2 Hz, 1H), 2.89-2.56 (m, 2H), 1.70 (d, J = 44.8 Hz, 3H), 0.80 (d, J = 7.1 Hz, 6H). | [M + H]⁺: 473.3 |
| 60 | 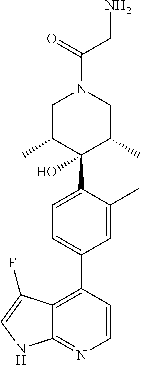 [SMILES: Cc1cc(-c2ccnc3[nH]cc(F)c23)ccc1[C@@]1(O)[C@H](C)CN(C(=O)CN)C[C@@H]1C] | 2-amino-1- ((3R,4s,5S)-4-(4- (3-fluoro-1H- pyrrolo[2,3- b]pyridin-4-yl)-2- methylphenyl)-4- hydroxy-3,5- dimethylpiperidin- 1-yl)ethan-1-one | ¹H NMR (400 MHz, DMSO-d₆) δ 11.62 (d, J = 2.9 Hz, 1H), 8.28 (d, J = 4.9 Hz, 1H), 8.00 (bs, 3H), 7.82 (d, J = 8.3 Hz, 1H), 7.52-7.40 (m, 3H), 7.15 (dd, J = 4.9, 2.8 Hz, 1H), 4.87 (bs, 1H), 4.25-4.15 (m, 1H), 4.14-3.96 (m, 1H), 3.92-3.80 (m, 1H), 3.43 (d, J = 13.0 Hz, 1H), 3.23-3.08 (m, 1H), 2.85- 2.74 (m, 1H), 2.67 (s, 1H), 2.46 (s, 2H), 2.40-2.29 (m, 1H), 2.25-1.94 (m, 1H), 0.69 (dd, J = 6.9, 2.8 Hz, 2H), 0.58 (dd, J = 6.8, 3.6 Hz, 4H). | [M + H]⁺: 411.3 |
| 61 | 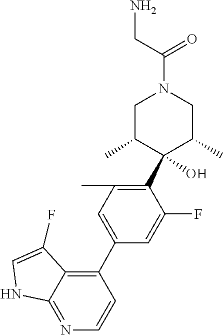 [SMILES: Cc1cc(-c2ccnc3[nH]cc(F)c23)cc(F)c1[C@@]1(O)[C@H](C)CN(C(=O)CN)C[C@@H]1C] | 2-amino-1- ((3R,4s,5S)-4-(2- fluoro-4-(3-fluoro- 1H-pyrrolo[2,3- b]pyridin-4-yl)-6- methylphenyl)-4- hydroxy-3,5- dimethylpiperidin- 1-yl)ethan-1-one | ¹H NMR (400 MHz, Methanol-d₄) δ 8.26 (d, J = 5.0 Hz, 1H), 7.32-7.27 (m, 2H), 7.25-7.13 (m, 2H), 4.30 (dd, J = 13.1, 4.4 Hz, 1H), 3.62-3.45 (m, 3H), 3.24 (t, J = 12.1 Hz, 1H), 2.92-2.78 (m, 1H), 2.76 (s, 3H), 2.59 (qt, J = 12.4, 5.0 Hz, 2H), 0.83 (dd, J = 7.1, 3.9 Hz, 6H). | [M + H]⁺: 429.2 |
| 62 | 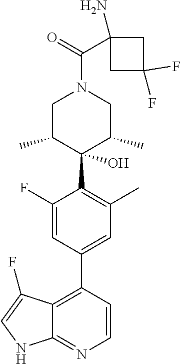 [SMILES: Cc1cc(-c2ccnc3[nH]cc(F)c23)cc(F)c1[C@@]1(O)[C@H](C)CN(C(=O)C2(N)CC(F)(F)C2)C[C@@H]1C] | (1-amino-3,3- difluorocyclobutyl) ((3S,4s,5R)-4-(2- fluoro-4-(3-fluoro- 1H-pyrrolo[2,3- b]pyridin-4-yl)-6- methylphenyl)-4- hydroxy-3,5- dimethylpiperidin- 1-yl)methanone | ¹H NMR (400 MHz, DMSO-d₆) δ 11.67 (s, 1H), 8.31 (d, J = 4.9 Hz, 1H), 7.54 (s, 1H), 7.26 (d, J = 15.0 Hz, 2H), 7.20 (d, J = 4.9 Hz, 1H), 4.61 (s, 1H), 4.23-4.07 (m, 2H), 3.69- 3.45 (m, 2H), 3.26-3.08 (m, 4H), 2.82-2.67 (m, 1H), 2.72 (s, 3H), 2.48- 2.36 (m, 1H), 0.74 (d, J = 6.8 Hz, 6H). | [M + H]⁺: 505.2 |
| 63 | 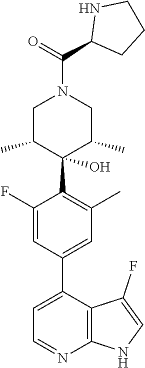 [SMILES: Cc1cc(-c2ccnc3[nH]cc(F)c23)cc(F)c1[C@@]1(O)[C@H](C)CN(C(=O)[C@@H]2CCCN2)C[C@@H]1C] | 4-(4-((3S,4R,5R)- 1-(L-prolyl)-4- hydroxy-3,5- dimethylpiperidin- 4-yl)-3-fluoro-5- methylphenyl)-3- fluoro-1H- pyrrolo[2,3- b]pyridine | ¹H NMR (400 MHz, DMSO-d₆) δ 11.69 (s, 1H), 9.26 (s, 1H), 8.51 (s, 1H), 8.32 (d, J = 4.9 Hz, 1H), 7.55 (s, 1H), 7.27 (d, J = 18.9 Hz, 2H), 7.21 (d, J = 4.9 Hz, 1H), 4.73 (d, J = 9.8 Hz, 1H), 4.66-4.58 (m, 1H), 4.19 (d, J = 12.9 Hz, 1H), 3.65-3.55 (m, 1H), 3.35-3.12 (m, 3H), 2.85 (t, J = 12.6 Hz, 1H), 2.73 (s, 3H), 2.46-2.35 (m, 2H), 1.94 (dq, J = 19.6, 7.2 Hz, 2H), 1.84- 1.66 (m, 1H), 0.78 (dd, J = 10.0, 6.3 Hz, 6H). | [M + H]⁺: 469.2 |
| 64 | 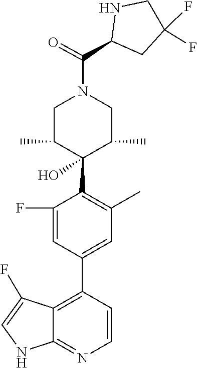 [SMILES: Cc1cc(-c2ccnc3[nH]cc(F)c23)cc(F)c1[C@@]1(O)[C@H](C)CN(C(=O)[C@@H]2CC(F)(F)CN2)C[C@@H]1C] | ((S)-4,4- difluoropyrrolidin- 2-yl)((3S,4R,5R)- 4-(2-fluoro-4-(3- fluoro-1H- pyrrolo[2,3- b]pyridin-4-yl)-6- methylphenyl)-4- hydroxy-3,5- dimethylpiperidin- 1-yl)methanone | ¹H NMR (400 MHz, Methanol-d₄) δ 8.29 (d, J = 5.1 Hz, 1H), 7.32 (d, J = 5.7 Hz, 2H), 7.31-7.16 (m, 2H), 5.13 (dt, J = 40.6, 8.8 Hz, 1H), 4.32 (dd, J = 13.0, 5.1 Hz, 1H), 3.86 (dp, J = 36.2, 12.6 Hz, 2H), 3.54-3.34 (m, 2H), 3.25- 3.06 (m, 1H), 3.06-2.93 (m, 1H), 2.78 (s, 3H), 2.74- 2.54 (m, 3H), 0.95-0.81 (m, 6H). | [M + H]⁺: 505.2 |
| 65 | 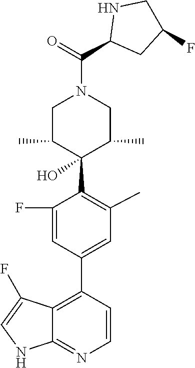 [SMILES: Cc1cc(-c2ccnc3[nH]cc(F)c23)cc(F)c1[C@@]1(O)[C@H](C)CN(C(=O)[C@@H]2C[C@H](F)CN2)C[C@@H]1C] | ((3S,4R,5R)-4-(2- fluoro-4-(3-fluoro- 1H-pyrrolo[2,3- b]pyridin-4-yl)-6- methylphenyl)-4- hydroxy-3,5- dimethylpiperidin- 1-yl)((2S,4S)-4- fluoropyrrolidin-2- yl)methanone | ¹H NMR (400 MHz, Methanol-d₄) δ 8.29 (d, J = 5.1 Hz, 1H), 7.32 (d, J = 8.1 Hz, 2H), 7.29-7.18 (m, 2H), 5.58-5.32 (m, 1H), 4.96-4.90 (m 1H), 4.36-4.27 (m, 1H), 3.86 (t, J = 15.5 Hz, 1H), 3.58- 3.38 (m, 3H), 3.00 (t, J = 12.6 Hz, 1H), 2.78 (s, 3H), 2.73-2.55 (m, 1H), 2.47- 2.23 (m, 1H), 0.95-0.81 (m, 6H). | [M + H]⁺: 487.2 |
| 66 | 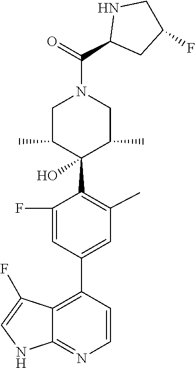 [SMILES: Cc1cc(-c2ccnc3[nH]cc(F)c23)cc(F)c1[C@@]1(O)[C@H](C)CN(C(=O)[C@@H]2C[C@@H](F)CN2)C[C@@H]1C] | ((3S,4R,5R)-4-(2- fluoro-4-(3-fluoro- 1H-pyrrolo[2,3- b]pyridin-4-yl)-6- methylphenyl)-4- hydroxy-3,5- dimethylpiperidin- 1-yl)((2S,4R)-4- fluoropyrrolidin-2- yl)methanone | ¹H NMR (400 MHz, Methanol-d₄) δ 8.36-8.22 (m, 1H), 7.31 (d, J = 7.4 Hz, 2H), 7.26-7.18 (m, 2H), 5.67-5.39 (m, 1H), 5.04 (dt, J = 38.6, 8.9 Hz, 1H), 4.35-4.27 (m, 1H), 3.71 (q, J = 13.2, 12.0 Hz, 1H), 3.63-3.52 (m, 1H), 3.42 (dd, J = 29.1, 15.5 Hz, 1H), 3.04-2.88 (m, 2H), 2.77 (s, 3H), 2.74-2.52 (m, 2H), 2.37-2.13 (m, 1H), 0.94-0.81 (m, 6H). | [M + H]⁺: 487.2 |
| 67 | 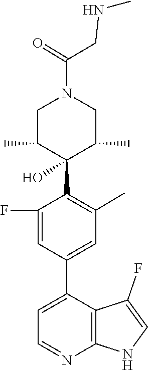 [SMILES: CNCC(=O)N1C[C@@H](C)[C@](O)(c2c(C)cc(-c3ccnc4[nH]cc(F)c34)cc2F)[C@@H](C)C1] | 1-((3S,4s,5R)-4-(2- fluoro-4-(3-fluoro- 1H-pyrrolo[2,3- b]pyridin-4-yl)-6- methylphenyl)-4- hydroxy-3,5- dimethylpiperidin- 1-yl)-2- (methylamino) ethan-1-one | ¹H NMR (400 MHz, Methanol-d₄) δ 8.29 (d, J = 5.0 Hz, 1H), 7.31 (d, J = 7.2 Hz, 2H), 7.26-7.19 (m, 2H), 4.30 (d, J = 12.9 Hz, 1H), 4.22 (d, J = 16.0 Hz, 1H), 4.07 (d, J = 16.0 Hz, 1H), 3.52-3.32 (m, 2H), 3.02-2.86 (m, 1H), 2.78 (s, 3H), 2.77 (s, 3H), 2.71-2.54 (m, 2H), 0.86 (t, J = 7.6 Hz, 6H). | [M + H]⁺: 443.2 |
| 68 | 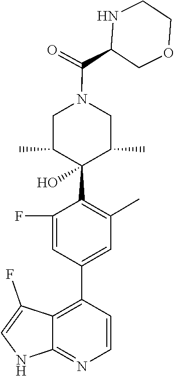 [SMILES: Cc1cc(-c2ccnc3[nH]cc(F)c23)cc(F)c1[C@@]1(O)[C@H](C)CN(C(=O)[C@@H]2COCCN2)C[C@@H]1C] | ((3S,4R,5R)-4-(2- fluoro-4-(3-fluoro- 1H-pyrrolo[2,3- b]pyridin-4-yl)-6- methylphenyl)-4- hydroxy-3,5- dimethylpiperidin- 1-yl)((S)- morpholin-3- yl)methanone | ¹H NMR (400 MHz, Methanol-d₄) δ 8.29 (d, J = 5.1 Hz, 1H), 7.32 (d, J = 6.3 Hz, 2H), 7.25 (s, 1H), 7.21 (d, J = 4.9 Hz, 1H), 4.83-4.68 (m, 1H), 4.34- 4.18 (m, 2H), 4.07 (d, J = 12.7 Hz, 1H), 3.85-3.55 (m, 3H), 3.50-3.34 (m, 3H), 2.94 (t, J = 12.4 Hz, 1H), 2.78 (s, 3H), 2.72- 2.64 (m, 1H), 2.64-2.51 (m, 2H), 0.90 (d, J = 6.5 Hz, 3H), 0.85 (d, J = 6.9 Hz, 3H). | [M + H]⁺: 485.3 |
| 69 | 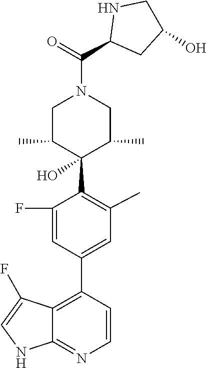 [SMILES: Cc1cc(-c2ccnc3[nH]cc(F)c23)cc(F)c1[C@@]1(O)[C@H](C)CN(C(=O)[C@@H]2C[C@@H](O)CN2)C[C@@H]1C] | ((3S,4R,5R)-4-(2- fluoro-4-(3-fluoro- 1H-pyrrolo[2,3- b]pyridin-4-yl)-6- methylphenyl)-4- hydroxy-3,5- dimethylpiperidin- 1-yl)((2S,4R)-4- hydroxypyrrolidin- 2-yl)methanone | | [M + H]⁺: 485.2 |
| 70 | 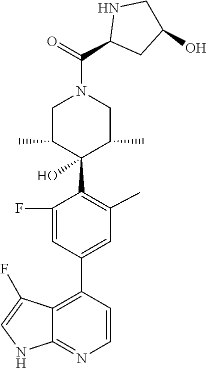 [SMILES: Cc1cc(-c2ccnc3[nH]cc(F)c23)cc(F)c1[C@@]1(O)[C@H](C)CN(C(=O)[C@@H]2C[C@H](O)CN2)C[C@@H]1C] | ((3S,4R,5R)-4-(2- fluoro-4-(3-fluoro- 1H-pyrrolo[2,3- b]pyridin-4-yl)-6- methylphenyl)-4- hydroxy-3,5- dimethylpiperidin- 1-yl)((2S,4S)-4- hydroxypyrrolidin- 2-yl)methanone | | [M + H]⁺: 485.2 |
| 310 | 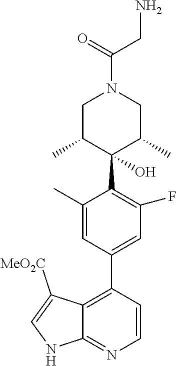 [SMILES: COC(=O)c1c[nH]c2nccc(-c3cc(C)c([C@@]4(O)[C@H](C)CN(C(=O)CN)C[C@@H]4C)c(F)c3)c12] | methyl 4-(3-fluoro- 4-((3S,4s,5R)-1- glycyl-4-hydroxy- 3,5- dimethylpiperidin- 4-yl)-5- methylphenyl)-1H- pyrrolo[2,3- b]pyridine-3- carboxylate | ¹H NMR (400 MHz, Methanol-d₄) δ 8.41-8.23 (m, 1H), 8.13 (d, J = 2.1 Hz, 1H), 7.14 (dd, J = 5.5, 2.7 Hz, 1H), 7.01 (d, J = 1.8 Hz, 1H), 6.95 (d, J = 13.9 Hz, 1H), 4.31 (dd, J = 12.6, 4.4 Hz, 1H), 3.61- 3.48 (m, 3H), 3.45 (s, 3H), 3.29-3.18 (m, 1H), 2.92- 2.78 (m, 1H), 2.72 (s, 3H), 2.66-2.50 (m, 1H), 1.32- 1.27 (m, 1H), 0.86 (dd, J = 6.9, 4.7 Hz, 6H). | [M + H]⁺: 469.2 |
| 311 | 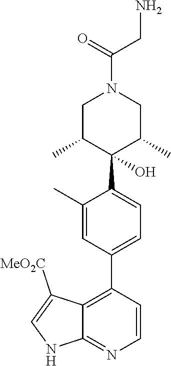 [SMILES: COC(=O)c1c[nH]c2nccc(-c3ccc([C@@]4(O)[C@H](C)CN(C(=O)CN)C[C@@H]4C)c(C)c3)c12] | methyl 4-(4- ((3S,4s,5R)-1- glycyl-4-hydroxy- 3,5- dimethylpiperidin- 4-yl)-3- methylphenyl)-1H- pyrrolo[2,3- b]pyridine-3- carboxylate | ¹H NMR (400 MHz, DMSO-d₆) δ 12.53 (s, 1H), 8.32 (dd, J = 4.9, 2.0 Hz, 1H), 8.16 (d, J = 3.0 Hz, 1H), 7.99 (bs, 3H), 7.75 (d, J = 8.2 Hz, 1H), 7.27- 7.11 (m, 3H), 4.82 (s, 1H), 4.26-4.16 (m, 1H), 4.14- 3.95 (m, 1H), 3.93-3.70 (m, 1H), 3.46-3.39 (d, J = 13.2 Hz, 1H), 3.23 (d, J = 2.1 Hz, 3H), 3.20-3.10 (m, 1H), 2.80 (q, J = 11.7 Hz, 1H), 2.63 (s, 1H), 2.42 (s, 2H), 2.28-1.90 (m, 1H), 0.76-0.49 (m, 6H). | [M + H]⁺: 451.1 |
| 312 | 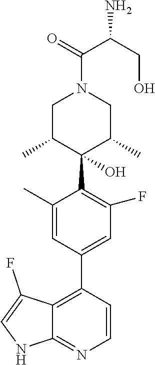 [SMILES: Cc1cc(-c2ccnc3[nH]cc(F)c23)cc(F)c1[C@@]1(O)[C@H](C)CN(C(=O)[C@H](N)CO)C[C@@H]1C] | (R)-2-amino-1- ((3S,4R,5R)-4-(2- fluoro-4-(3-fluoro- 1H-pyrrolo[2,3- b]pyridin-4-yl)-6- methylphenyl)-4- hydroxy-3,5- dimethylpiperidin- 1-yl)-3- hydroxypropan-1- one | ¹H NMR (400 MHz, DMSO-d₆) δ 11.68 (s, 1H), 8.30 (d, J = 4.9 Hz, 1H), 8.08 (bs, 3H), 7.53 (t, J = 2.4 Hz, 1H), 7.29-7.21 (m, 2H), 7.19 (d, J = 4.9 Hz, 1H), 5.43 (bs, 1H), 4.68 (d, J = 4.2 Hz, 1H), 4.59-4.50 (m, 1H), 4.44- 4.37 (m, 1H), 4.23-4.16 (m, 1H), 3.75-3.55 (m, 6H), 3.19 (t, J = 12.9 Hz, 1H), 2.83-2.74 (m, 1H), 2.71 (s, 3H), 0.75 (dd, J = 6.8, 2.4 Hz, 6H). | [M + H]⁺: 459.1 |

### Compound 71

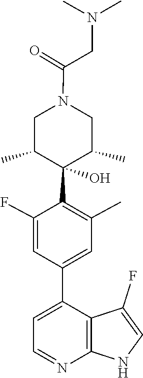 [SMILES: Cc1cc(-c2ccnc3[nH]cc(F)c23)cc(F)c1[C@@]1(O)[C@H](C)CN(C(=O)CN(C)C)C[C@@H]1C]

### 2-(dimethylamino)-1-((3S,4s,5R)-4-(2-fluoro-4-(3-fluoro-1H-pyrrolo[2,3-b]pyridin-4-yl)-6-methylphenyl)-4-hydroxy-3,5-dimethylpiperid-1-yl)ethan-1-one

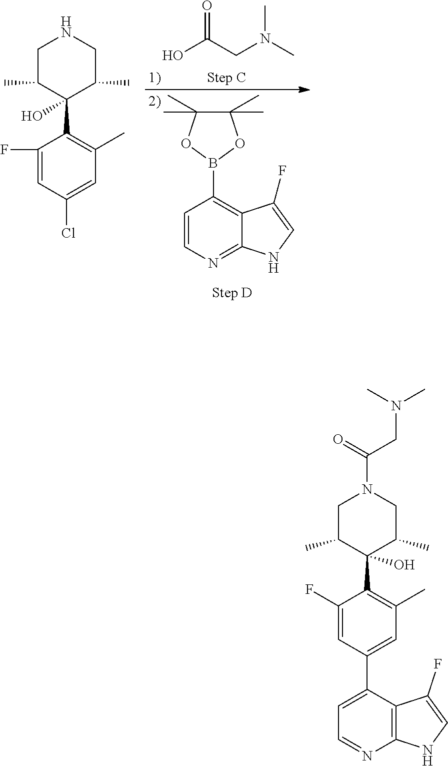

Step C: A vial was charged with (3S,4s,5R)-4-(4-chloro-2-fluoro-6-methylphenyl)-3,5-dimethylpiperidin-4-ol (0.070 g, 0.26 mmol), dimethylglycine (0.032 g, 0.31 mmol) and 1-[bis(dimethylamino)methylene]-1H-1,2,3-triazolo[4,5-b]pyridinium 3-oxide hexafluorophosphate (0.118 g, 0.31 mmol, HATU). Tetrahydrofuran (4.0 ml) was added, followed by diisopropylethylamine (0.090 ml, 0.515 mmol). The reaction was then stirred at room temperature for 3 hours. The reaction was then partitioned between aqueous saturated sodium bicarbonate and ethyl acetate. The organic phase was taken, and the solvent was removed by rotary evaporation to give the crude product. Purification by silica gel chromatography gave the product. LC-MS (m/z [M+H]⁺): 357.2

Step D: A microwave vial was charged with 1-((3S,4s,5R)-4-(4-chloro-2-fluoro-6-methylphenyl)-4-hydroxy-3,5-dimethylpiperidin-1-yl)-2-(dimethylamino)ethan-1-one (0.050 g, 0.140 mmol), 3-fluoro-4-(4,4,5,5-tetramethyl-1,3,2-dioxaborolan-2-yl)-1H-pyrrolo[2,3-b]pyridine (0.05 g, 0.191 mmol), potassium phosphate (tribasic, K₃PO₄, 0.100 g, 0.471 mmol), and Chloro(2-dicyclohexylphosphino-2′,4′,6′-triisopropyl-1,1′-biphenyl)[2-(2′-amino-1,1′-biphenyl)]palladium(II) (Xphos Pd G2, 15.0 mg, 0.019 mmol). The vial was capped. Then, tetrahydrofuran (1.0 mL) was added, followed by water (1.0 ml). The content of the reaction vial was purged with nitrogen via bubbling through the solvent. The reaction was then heated in the microwave reactor at 100° C. for 45 min. The reaction was cooled to room temperature and poured into an aqueous solution of sodium bicarbonate. The mixture was extracted with ethyl acetate, the organic phases were partitioned, and the organic solvent removed by rotary evaporation. The crude product was dissolved in methanol (1.0 mL) and purified by reverse phase HPLC (5% to 100% acetonitrile in water, containing 0.1% formic acid) to give the title product. ¹H NMR (400 MHz, DMSO-d₆) δ 11.68 (s, 1H), 8.48-8.14 (m, 1H), 7.54 (s, 1H), 7.29-7.23 (m, 2H), 7.21 (d, J=4.9 Hz, 1H), 4.58 (s, 1H), 4.17 (d, J=12.6 Hz, 1H), 3.70 (d, J=12.9 Hz, 1H), 3.51-3.39 (m, 1H), 3.11 (t, J=12.4 Hz, 2H), 2.72 (s, 3H), 2.70-2.60 (if, 1H) 2.30 (s, 6H), 0.74 (d, J=6.8 Hz, 6H). LC-MS (m/z [M+H]⁺): 457.2

### Compounds 72-74

Compounds 72-74 and 313 were prepared using an analogous procedure to that of Compound 71, and are shown in Table 13.

TABLE 13

| | | | | LCMS |
|---|---|---|---|---|
| Compound | Structure | IUPAC name | ¹H NMR | m/z |
| 72 | 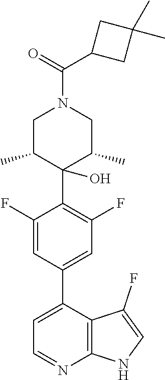 | ((3S,5R)-4-(2,6- difluoro-4-(3-fluoro- 1H-pyrrolo[2,3- b]pyridin-4- yl)phenyl)-4-hydroxy- 3,5-dimethylpiperidin- 1-yl)(3,3- dimethylcyclobutyl) methanone | ¹H NMR (400 MHz, DMSO- d₆) δ 11.75 (s, 1H), 8.33 (d, J = 4.9 Hz, 1H), 7.62-7.48 (m, 1H), 7.41-7.29 (m, 2H), 7.25 (d, J = 4.9 Hz, 1H), 4.97 (s, 1H), 4.17 (d, J = 12.7 Hz, 1H), 3.42 (d, J = 13.0 Hz, 1H), 3.36-3.66 (m, 1H), 3.07 (t, J = 12.3 Hz, 1H), 2.75-2.60 (m, 1H), 2.28- 2.13 (m, 2H), 2.06-1.97 (m, 1H), 1.92 (d, J = 8.8 Hz, 2H), 1.85 (t, J = 10.0 Hz, 1H), 1.17 (s, 3H), 1.03 (s, 3H), 0.79 (dd, J = 6.9, 2.3 Hz, 6H). | [M + H]⁺: 486.3 |
| 73 | 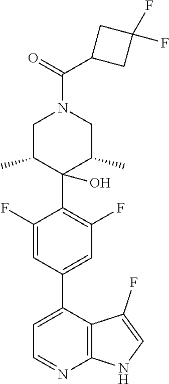 | (3,3- difluorocyclobutyl) ((3S,5R)-4-(2-fluoro-4- (3-fluoro-1H- pyrrolo[2,3-b]pyridin- 4-yl)-6- methylphenyl)-4- hydroxy-3,5- dimethylpiperidin-1- yl)methanone | ¹H NMR (400 MHz, DMSO- d₆) δ 11.72-11.60 (m, 1H), 8.31 (d, J = 4.9 Hz, 1H), 7.54 (s, 1H), 7.25 (d, J = 15.4 Hz, 2H), 7.20 (d, J = 5.0 Hz, 1H), 4.60 (s, 1H), 4.18 (d, J = 12.7 Hz, 1H), 3.51 (d, J = 13.2 Hz, 1H), 3.36 (t, J = 10.2 Hz, 1H), 3.12 (t, J = 12.7 Hz, 1H), 2.89-2.66 (m, 6H), 2.44-2.26 (m, 1H), 0.75 (t, J = 6.3 Hz, 6H). | [M + H]⁺: 490.2 |
| 74 | 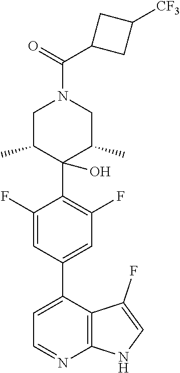 [SMILES: C[C@@H]1CN(C(=O)C2CC(C(F)(F)F)C2)C[C@H](C)C1(O)c1c(F)cc(-c2ccnc3[nH]cc(F)c23)cc1F] | ((3S,5R)-4-(2,6- difluoro-4-(3-fluoro- 1H-pyrrolo[2,3- b]pyridin-4- yl)phenyl)-4-hydroxy- 3,5-dimethylpiperidin- 1-yl)(3- (trifluoromethyl) cyclobutyl)methanone | ¹H NMR (400 MHz, DMSO- d₆) δ 11.75 (s, 1H), 8.34 (d, J = 4.9 Hz, 1H), 7.59 (d, J = 2.9 Hz, 1H), 7.34 (d, J = 13.1 Hz, 2H), 7.26 (d, J = 4.9 Hz, 1H), 5.01 (s, 1H), 4.18 (t, J = 15.2 Hz, 1H), 3.53-3.47 (m, 1H), 3.18-3.04 (m, 2H), 2.76-2.66 (m, 1H), 2.40- 2.17 (m, 4H), 0.81 (d, J = 6.9 Hz, 6H). | [M + H]⁺: 526.2 |
| 313 | 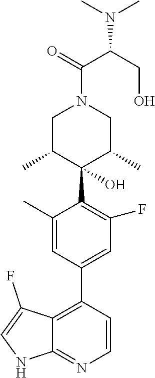 [SMILES: Cc1cc(-c2ccnc3[nH]cc(F)c23)cc(F)c1[C@@]1(O)[C@H](C)CN(C(=O)[C@@H](CO)N(C)C)C[C@@H]1C] | (R)-2- (dimethylamino)-1- ((3S,4S,5R)-4-(2- fluoro-4-(3-fluoro-1H- pyrrolo[2,3-b]pyridin- 4-yl)-6- methylphenyl)-4- hydroxy-3,5- dimethylpiperidin-1- yl)-3-hydroxypropan- 1-one | ¹H NMR (400 MHz, DMSO- d₆) δ 11.69 (s, 1H), 9.55 (bs, 1H), 8.31 (d, J = 4.9 Hz, 1H), 7.53 (d, J = 2.8 Hz, 1H), 7.26 (d, J = 19.1 Hz, 2H), 7.20 (dd, J = 4.9, 1.7 Hz, 1H), 5.71-5.57 (m, 1H), 4.72 (s, 1H), 4.71-4.48 (m, 1H), 4.25-4.16 (m, 1H), 3.90-3.77 (m, 2H), 3.64- 3.53 (m, 1H), 3.32-3.18 (m, 1H), 2.88-2.74 (m, 6H), 2.72 (s, 3H), 2.46-2.36 (m, 2H), 0.77 (dd, J = 6.8, 4.0 Hz, 6H). | [M + H]⁺: 487.1 |

### Compound 75

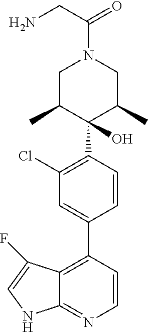 [SMILES: C[C@@H]1CN(C(=O)CN)C[C@H](C)[C@@]1(O)c1ccc(-c2ccnc3[nH]cc(F)c23)cc1Cl]

### 2-amino-1-((3R,4s,S5S)-4-(2-chloro-4-(3-fluoro-1H-pyrrolo[2,3-b]pyridin-4-yl)phenyl)-4-hydroxy-3,5-dimethylpiperid-1-yl)ethan-1-one

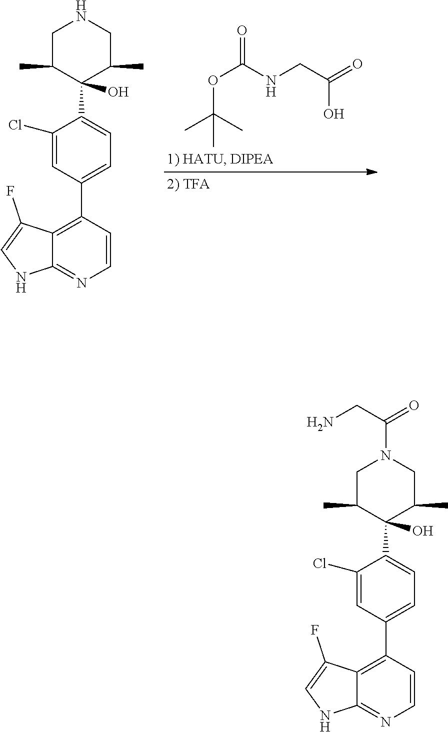 [SMILES: C[C@@H]1CNC[C@H](C)[C@@]1(O)c1ccc(-c2ccnc3[nH]cc(F)c23)cc1Cl ; CC(C)(C)OC(=O)NCC(=O)O ; C[C@@H]1CN(C(=O)CN)C[C@H](C)[C@@]1(O)c1ccc(-c2ccnc3[nH]cc(F)c23)cc1Cl]

Step 1: A solution containing (3S,4s,5R)-4-(2-chloro-4-(3-fluoro-1H-pyrrolo[2,3-b]pyridin-4-yl)phenyl)-3,5-dimethylpiperidin-4-ol (30 mg, 0.080 mmol) and tert-butoxycarbonyl)glycine (16.9 mg, 0.096 mmol) in dimethylformamide (0.8 mL) was treated with 1-[bis(dimethylamino)methylene]-1H-1,2,3-triazolo[4,5-b]pyridimin 3-oxide hexafluorophosphate (36.6 mg, 0.096 mmol, HATU) and diisopropylethylamine (21.02 μL, 0.120 mmol) at room temperature for 0.5 hours. The reaction mixture was partitioned between saturated aqueous sodium bicarbonate and tert-butylmethyl ether. The aqueous phase was then extracted with tertbutylmethyl ether (2×). The combined organic layer was washed with brine, dried over anhydrous sodium sulfate, filtered, and concentrated under diminished pressure to give the crude product.

Step 2: The crude product was dissolved in dichloromethane (0.8 mL) and treated with trifluoroacetic acid (0.12 mL, 1.6 mmol) at room temperature for 1.5 hours. The reaction mixture was then concentrated under reduced pressure. The crude product was dissolved in methanol (1.0 mL) and purified by reverse phase HPLC (5% to 100% acetonitrile in water, containing 0.1% formic acid) to give the title product. ¹H NMR (400 MHz, DMSO-d₆) δ 11.73 (d, J=2.8 Hz, 1H), 8.33 (d, J=4.9 Hz, 1H), 8.01 (s, 3H), 7.96 (d, J=8.8 Hz, 1H), 7.75-7.65 (m, 2H), 7.56 (d, J=2.4 Hz, 1H), 7.23 (d, J=4.9 Hz, 1H), 5.17 (s, 1H), 4.26 (d, J=10.0 Hz, 1H), 4.09 (dd, J=16.5, 5.9 Hz, 1H), 3.87 (dd, J=16.6, 5.2 Hz, 1H), 3.54-3.45 (m, 1H), 3.16 (t, J=12.4 Hz, 1H), 3.01-2.92 (m, 1H), 2.87-2.74 (m, 2H), 0.60 (d, J=6.4 Hz, 6H). LC-MS (m/z [M+H]⁺): 431.1

### Compounds 76-87

Compounds 76 to 87 and 314 to 317 were prepared using an analogous procedure to that of Compound 75, and are shown in Table 14.

TABLE 14

| | | | | LCMS |
|---|---|---|---|---|
| Compound | Structure | IUPAC name | ¹H NMR | m/z |
| 76 | 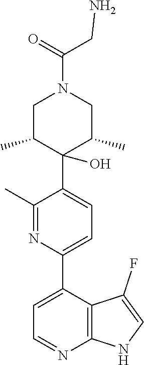 [SMILES: Cc1nc(-c2ccnc3[nH]cc(F)c23)ccc1C1(O)[C@H](C)CN(C(=O)CN)C[C@@H]1C] | 2-amino-1- ((3S,5R)-4-(6-(3- fluoro-1H- pyrrolo[2,3- b]pyridin-4-yl)-2- methylpyridin-3- yl)-4-hydroxy-3,5- dimethylpiperidin- 1-yl)ethan-1-one | ¹H NMR (400 MHz, DMSO- d₆) δ 11.68 (s, 1H), 8.35 (d, J = 4.9 Hz, 1H), 8.18 (d, J = 8.4 Hz, 1H), 7.69 (d, J = 26.8 Hz, 1H), 7.55 (s, 1H), 7.49 (d, J = 4.9 Hz, 1H), 5.02 (s, 1H), 4.26 (d, J = 13.0 Hz, 1H), 3.97 (d, J = 14.0 Hz, 1H), 3.77 (t, J = 16.7 Hz, 1H), 3.49 (d, J = 14.0 Hz, 1H), 3.22-3.12 (m, 1H), 2.88 (s, 1H), 2.68 (s, 2H), 2.45-2.35 (m, 1H), 2.25-2.10 (m, 1H), 2.06- 1.95 (m, 1H), 0.69 (s, 2H), 0.60 (d, J = 6.4 Hz, 4H). | [M + H]⁺: 412.2 |
| 77 | 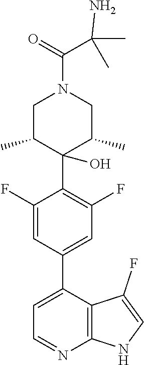 [SMILES: C[C@@H]1CN(C(=O)C(C)(C)N)C[C@H](C)C1(O)c1c(F)cc(-c2ccnc3[nH]cc(F)c23)cc1F] | 2-amino-1- ((3S,5R)-4-(2,6- difluoro-4-(3- fluoro-1H- pyrrolo[2,3- b]pyridin-4- yl)phenyl)-4- hydroxy-3,5- dimethylpiperidin- 1-yl)-2- methylpropan-1- one | ¹H NMR (400 MHz, DMSO- d₆) δ 11.76 (s, 1H), 8.34 (d, J = 4.9 Hz, 1H), 7.59 (s, 1H), 7.34 (d, J = 13.0 Hz, 2H), 7.26 (d, J = 4.9 Hz, 1H), 5.00 (s, 1H), 4.41-4.28 (m, 2H), 3.17 (s, 1H), 3.02- 2.85 (m, 1H), 2.36-2.24 (m, 2H), 1.39 (s, 6H), 0.81 (d, J = 6.9 Hz, 6H). | [M + H]⁺: 461.2 |
| 78 | 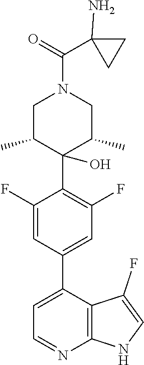 [SMILES: C[C@@H]1CN(C(=O)C2(N)CC2)C[C@H](C)C1(O)c1c(F)cc(-c2ccnc3[nH]cc(F)c23)cc1F] | (1- aminocyclopropyl) ((3S,5R)-4-(2,6- difluoro-4-(3- fluoro-1H- pyrrolo[2,3- b]pyridin-4- yl)phenyl)-4- hydroxy-3,5- dimethylpiperidin- 1-yl)methanone | ¹H NMR (400 MHz, DMSO- d₆) δ 11.75 (s, 1H), 8.46- 8.19 (m, 1H), 7.58 (dd, J = 2.9, 2.0 Hz, 1H), 7.33 (dd, J = 12.2, 2.6 Hz, 2H), 7.24 (d, J = 4.9 Hz, 1H), 4.99 (d, J = 1.8 Hz, 1H), 4.06 (dd, J = 9.0, 4.4 Hz, 2H), 3.07-2.84 (m, 2H), 2.39-2.22 (m, 2H), 0.87-0.82 (m, 2H), 0.80 (d, J = 6.9 Hz, 6H), 0.75-0.70 (m, 2H). | [M + H]⁺: 459.2 |
| 79 | 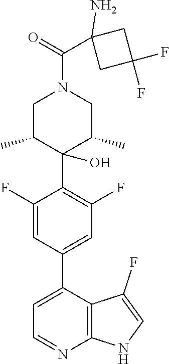 [SMILES: C[C@@H]1CN(C(=O)C2(N)CC(F)(F)C2)C[C@H](C)C1(O)c1c(F)cc(-c2ccnc3[nH]cc(F)c23)cc1F] | (1-amino-3,3- difluorocyclobutyl) ((3S,5R)-4-(2,6- difluoro-4-(3- fluoro-1H- pyrrolo[2,3- b]pyridin-4- yl)phenyl)-4- hydroxy-3,5- dimethylpiperidin- 1-yl)methanone | ¹H NMR (400 MHz, DMSO- d₆) δ 11.76 (s, 1H), 8.34 (d, J = 4.9 Hz, 1H), 7.59 (s, 1H), 7.34 (d, J = 13.3 Hz, 2H), 7.26 (d, J = 4.9 Hz, 1H), 5.02 (s, 1H), 4.15 (d, J = 12.7 Hz, 1H), 3.66 (d, J = 13.2 Hz, 1H), 3.23-3.06 (m, 2H), 2.80-2.70 (m, 1H), 2.69-2.56 (m, 2H), 2.47-2.36 (m, 3H), 2.34- 2.24 (m, 1H), 0.80 (s, 6H). | [M + H]⁺: 509.2 |
| 80 | 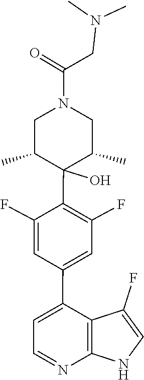 [SMILES: C[C@@H]1CN(C(=O)CN(C)C)C[C@H](C)C1(O)c1c(F)cc(-c2ccnc3[nH]cc(F)c23)cc1F] | 1-((3S,5R)-4-(2,6- difluoro-4-(3- fluoro-1H- pyrrolo[2,3- b]pyridin-4- yl)phenyl)-4- hydroxy-3,5- dimethylpiperidin- 1-yl)-2- (dimethylamino) ethan-1-one | ¹H NMR (400 MHz, DMSO- d₆) δ 11.76 (s, 1H), 8.34 (d, J = 4.9 Hz, 1H), 7.68-7.55 (m, 1H), 7.34 (dd, J = 12.3, 2.6 Hz, 2H), 7.26 (d, J = 4.9 Hz, 1H), 4.99 (s, 1H), 4.17 (d, J = 12.7 Hz, 1H), 3.72 (d, J = 13.2 Hz, 1H), 3.19-3.03 (m, 3H), 2.69 (t, J = 11.9 Hz, 1H), 2.45-2.34 (m, 1H), 2.31-2.20 (m, 1H), 2.25 (s, 6H), 0.80 (dd, J = 6.9, 2.8 Hz, 6H). | [M + H]⁺: 461.2 |
| 81 | 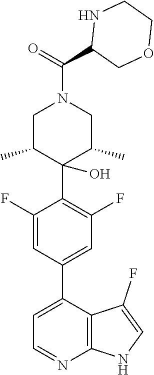 | ((3S,5R)-4-(2,6- difluoro-4-(3- fluoro-1H- pyrrolo[2,3- b]pyridin-4- yl)phenyl)-4- hydroxy-3,5- dimethylpiperidin- 1-yl)((S)- morpholin-3- yl)methanone | ¹H NMR (400 MHz, DMSO- d₆) δ 11.76 (s, 1H), 8.34 (d, J = 4.9 Hz, 1H), 7.59 (s, 1H), 7.35 (d, J = 13.1 Hz, 2H), 7.26 (d, J = 4.9 Hz, 1H), 5.06 (s, 1H), 4.17 (d, J = 12.3 Hz, 1H), 4.12-3.97 (m, 1H), 3.96-3.66 (m, 2H), 3.48-3.33 (m, 1H), 3.22-3.12 (m, 1H), 2.96- 2.82 (m, 2H), 2.79-2.63 (m, 1H), 2.44-2.12 (m, 3H), 0.83 (t, J = 6.6 Hz, 6H). | [M + H]⁺: 489.2 |
| 82 | 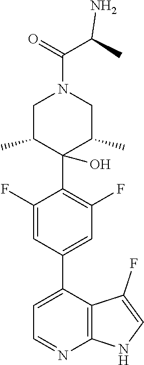 [SMILES: C[C@H](N)C(=O)N1C[C@@H](C)C(O)(c2c(F)cc(-c3ccnc4[nH]cc(F)c34)cc2F)[C@@H](C)C1] | (2S)-2-amino-1- ((3S,5R)-4-(2,6- difluoro-4-(3- fluoro-1H- pyrrolo[2,3- b]pyridin-4- yl)phenyl)-4- hydroxy-3,5- dimethylpiperidin- 1-yl)propan-1-one | ¹H NMR (400 MHz, DMSO- d₆) δ 11.77 (s, 1H), 8.34 (d, J = 4.9 Hz, 1H), 7.59 (s, 1H), 7.35 (d, J = 13.1 Hz, 2H), 7.26 (d, J = 5.1 Hz, 1H), 5.07 (s, 1H), 4.28-4.01 (m, 1H), 3.64 (d, J = 13.1 Hz, 1H), 3.23-3.05 (m, 1H), 2.81-2.63 (m, 1H), 2.44- 2.17 (m, 2H), 1.21 (d, J = 19.8 Hz, 3H), 0.83 (s, 6H). | [M + H]⁺: 447.2 |
| 83 | 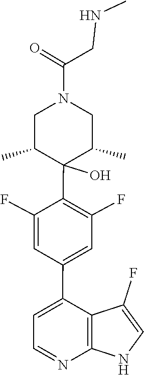 [SMILES: CNCC(=O)N1C[C@@H](C)C(O)(c2c(F)cc(-c3ccnc4[nH]cc(F)c34)cc2F)[C@@H](C)C1] | 1-((3S,5R)-4-(2,6- difluoro-4-(3- fluoro-1H- pyrrolo[2,3- b]pyridin-4- yl)phenyl)-4- hydroxy-3,5- dimethylpiperidin- 1-yl)-2- (methylamino) ethan-1-one | ¹H NMR (400 MHz, DMSO- d₆) δ 11.76 (s, 1H), 8.34 (d, J = 4.9 Hz, 1H), 7.59 (s, 1H), 7.41-7.31 (m, 2H), 7.26 (d, J = 4.9 Hz, 1H), 5.07 (s, 1H), 4.19 (d, J = 12.4 Hz, 1H), 3.82 (d, J = 16.1 Hz, 1H), 3.65 (d, J = 15.8 Hz, 1H), 3.46 (d, J = 13.1 Hz, 1H), 3.23-3.06 (m, 1H), 2.72 (q, J = 16.5, 14.6 Hz, 1H), 2.43 (s, 3H), 2.41-2.31 (m, 1H), 2.30-2.19 (m, 1H), 0.81 (d, J = 6.9 Hz, 6H). | [M + H]⁺: 447.2 |
| 84 | 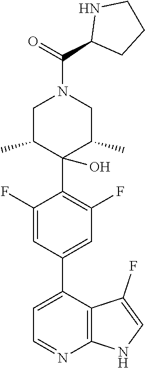 [SMILES: C[C@@H]1CN(C(=O)[C@@H]2CCCN2)C[C@H](C)C1(O)c1c(F)cc(-c2ccnc3[nH]cc(F)c23)cc1F] | 4-(4-((3S,5R)-1-(L- prolyl)-4-hydroxy- 3,5- dimethylpiperidin- 4-yl)-3,5- difluorophenyl)-3- fluoro-1H- pyrrolo[2,3- b]pyridine | ¹H NMR (400 MHz, DMSO- d₆) δ 11.77 (s, 1H), 8.34 (d, J = 4.9 Hz, 1H), 7.59 (s, 1H), 7.39-7.31 (m, 2H), 7.26 (dd, J = 5.0, 1.9 Hz, 1H), 5.09 (s, 1H), 4.33-4.07 (m, 2H), 3.63 (d, J = 13.2 Hz, 1H), 3.20-3.08 (m, 1H), 2.90-2.65 (m, 1H), 2.41- 2.13 (m, 4H), 1.85-1.54 (m, 4H), 0.82 (d, J = 7.1 Hz, 6H). | [M + H]⁺: 473.3 |
| 85 | 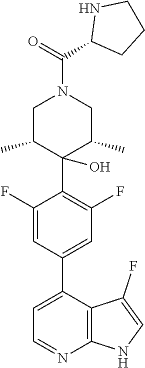 [SMILES: C[C@@H]1CN(C(=O)[C@H]2CCCN2)C[C@H](C)C1(O)c1c(F)cc(-c2ccnc3[nH]cc(F)c23)cc1F] | 4-(4-((3S,5R)-1- (D-prolyl)-4- hydroxy-3,5- dimethylpiperidin- 4-yl)-3,5- difluorophenyl)-3- fluoro-1H- pyrrolo[2,3- b]pyridine | ¹H NMR (400 MHz, DMSO- d₆) δ 11.77 (s, 1H), 8.34 (d, J = 4.9 Hz, 1H), 7.59 (s, 1H), 7.40-7.32 (m, 2H), 7.26 (d, J = 4.8 Hz, 1H), 5.09 (s, 1H), 4.33 (s, 1H), 4.19 (d, J = 11.8 Hz, 1H), 3.63 (d, J = 13.2 Hz, 1H), 3.16 (t, J = 11.1 Hz, 1H), 2.93-2.85 (m, 1H), 2.78 (t, J = 12.3 Hz, 1H), 2.43-2.16 (m, 3H), 1.85-1.52 (m, 3H), 0.88- 0.78 (m, 6H). | [M + H]⁺: 473.3 |
| 86 | 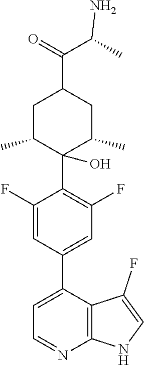 [SMILES: C[C@@H]1CC(C(=O)[C@@H](C)N)C[C@H](C)C1(O)c1c(F)cc(-c2ccnc3[nH]cc(F)c23)cc1F] | (2R)-2-amino-1- ((3S,5R)-4-(2,6- difluoro-4-(3- fluoro-1H- pyrrolo[2,3- b]pyridin-4- yl)phenyl)-4- hydroxy-3,5- dimethylpiperidin- 1-yl)propan-1-one | | [M + H]⁺: 447.2 |
| 87 | 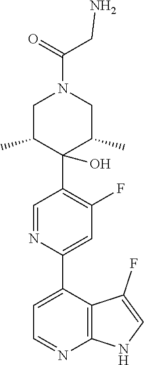 [SMILES: C[C@@H]1CN(C(=O)CN)C[C@H](C)C1(O)c1cnc(-c2ccnc3[nH]cc(F)c23)cc1F] | 2-amino-1- ((3S,5R)-4-(4- fluoro-6-(3-fluoro- 1H-pyrrolo[2,3- b]pyridin-4- yl)pyridin-3-yl)-4- hydroxy-3,5- dimethylpiperidin- 1-yl)ethan-1-one | ¹H NMR (400 MHz, DMSO- d₆) δ 11.79 (s, 1H), 8.91 (d, J = 11.2 Hz, 1H), 8.39 (d, J = 5.0 Hz, 1H), 8.01 (s, 3H), 7.70 (d, J = 12.9 Hz, 1H), 7.61 (s, 1H), 7.52 (d, J = 5.0 Hz, 1H), 5.42 (s, 1H), 4.26 (d, J = 12.8 Hz, 1H), 4.10 (d, J = 15.6 Hz, 1H), 3.87 (d, J = 16.1 Hz, 1H), 3.49 (d, J = 12.8 Hz, 1H), 3.17 (t, J = 12.6 Hz, 1H), 2.80 (t, J = 12.4 Hz, 1H), 2.39-2.30 (m, 1H), 2.26-2.16 (m, 1H), 0.78-0.54 (m, 6H). | [M + H]⁺: 416.2 |
| 314 | 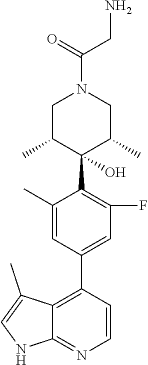 [SMILES: Cc1cc(-c2ccnc3[nH]cc(C)c23)cc(F)c1[C@@]1(O)[C@H](C)CN(C(=O)CN)C[C@@H]1C] | 2-amino-1- ((3S,4s,5R)-4-(2- fluoro-6-methyl-4- (3-methyl-1H- pyrrolo[2,3- b]pyridin-4- yl)phenyl)-4- hydroxy-3,5- dimethylpiperidin-1- yl)ethan-1-one | ¹H NMR (400 MHz, DMSO- d₆) δ 11.52 (s, 1H), 8.20 (d, J = 4.9 Hz, 1H), 8.00 (s, 3H), 7.28 (dd, J = 2.5, 1.3 Hz, 1H), 7.10- 7.02 (m, 2H), 6.95 (d, J = 4.9 Hz, 1H), 4.67 (s, 1H), 4.21 (dd, J = 12.8, 4.4 Hz, 1H), 4.13- 4.03 (m, 1H), 3.92-3.81 (m, 1H), 3.42 (d, J = 12.0 Hz, 1H), 3.16 (t, J = 12.5 Hz, 1H), 2.80 (t, J = 12.2 Hz, 1H), 2.71 (s, 3H), 2.46-2.35 (m, 1H), 1.87 (d, J = 1.1 Hz, 3H), 0.77 (dd, J = 6.9, 3.0 Hz, 7H). | [M + H]⁺: 425.1 |
| 315 | 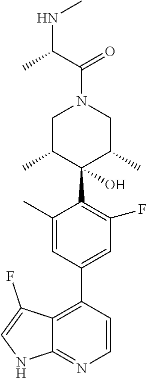 [SMILES: CN[C@@H](C)C(=O)N1C[C@@H](C)[C@](O)(c2c(C)cc(-c3ccnc4[nH]cc(F)c34)cc2F)[C@@H](C)C1] | (S)-1-((3S,4R,5R)-4- (2-fluoro-4-(3- fluoro-1H- pyrrolo[2,3- b]pyridin-4-yl)-6- methylphenyl)-4- hydroxy-3,5- dimethylpiperidin-1- yl)-2- (methylamino) propan-1-one | ¹H NMR (400 MHz, DMSO- d₆) δ 11.70 (s, 1H), 8.87 (bs, 1H), 8.32 (d, J = 4.9 Hz, 1H), 7.55 (t, J = 2.4 Hz, 1H), 7.31-7.25 (m, 2H), 7.21 (d, J = 4.9 Hz, 1H), 4.74 (d, J = 12.0 Hz, 1H), 4.57-4.36 (m, 1H), 4.25- 4.19 (m, 1H), 3.59-3.48 (m, 1H), 3.28-3.16 (m, 3H), 2.82 (t, J = 12.3 Hz, 1H), 2.73 (s, 3H), 2.54 (d, J = 6.7 Hz, 3H), 1.37 (dd, J = 11.4, 6.8 Hz, 3H), 0.83-0.73 (m, 6H). | [M + H]⁺: 457.1 |
| 316 | 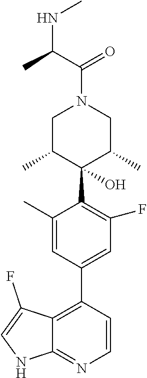 [SMILES: CN[C@H](C)C(=O)N1C[C@@H](C)[C@](O)(c2c(C)cc(-c3ccnc4[nH]cc(F)c34)cc2F)[C@@H](C)C1] | (R)-1-((3S,4S,5R)-4- (2-fluoro-4-(3- fluoro-1H- pyrrolo[2,3- b]pyridin-4-yl)-6- methylphenyl)-4- hydroxy-3,5- dimethylpiperidin-1- yl)-2- (methylamino) propan-1-one | ¹H NMR (400 MHz, DMSO- d₆) δ 11.68 (d, J = 2.8 Hz, 1H), 8.84 (bs, 1H), 8.30 (d, J = 4.9 Hz, 1H), 7.53 (d, J = 2.5 Hz, 1H), 7.25 (d, J = 19.5 Hz, 2H), 7.19 (d, J = 4.9 Hz, 1H), 4.71 (d, J = 12.1 Hz, 1H), 4.48 (dq, J = 39.2, 6.7 Hz, 1H), 4.23-4.16 (m, 1H), 3.57-3.46 (m, 3H), 3.25-3.16 (m, 1H), 2.80 (t, J = 12.1 Hz, 1H), 2.71 (s, 3H), 2.55- 2.50 (m, 3H), 1.35 (dd, J = 11.4, 6.8 Hz, 3H), 0.82-0.71 (m, 6H). | [M + H]⁺: 457.1 |
| 317 | 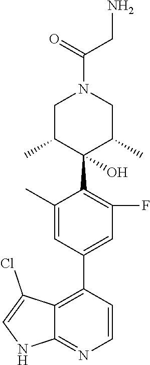 [SMILES: Cc1cc(-c2ccnc3[nH]cc(Cl)c23)cc(F)c1[C@@]1(O)[C@H](C)CN(C(=O)CN)C[C@@H]1C] | 2-amino-1- ((3S,4s,5R)-4-(4-(3- chloro-1H- pyrrolo[2,3- b]pyridin-4-yl)-2- fluoro-6- methylphenyl)-4- hydroxy-3,5- dimethylpiperidin-1- yl)ethan-1-one | ¹H NMR (400 MHz, DMSO- d₆) δ 12.17 (s, 1H), 8.30 (d, J = 4.8 Hz, 1H), 7.98 (bs, 3H), 7.69 (d, J = 2.8 Hz, 1H), 7.11-7.05 (m, 3H), 4.65 (s, 1H), 4.19 (dd, J = 11.5, 3.7 Hz, 1H), 4.06 (dd, J = 16.4, 5.8 Hz, 1H), 3.22-3.09 (m, 1H), 2.87-2.72 (m, 1H), 2.69 (s, 3H), 2.43-2.33 (m, 1H), 0.75 (dd, J = 6.9, 2.8 Hz, 6H). | [M + H]⁺: 445.1 |

### Compound 88

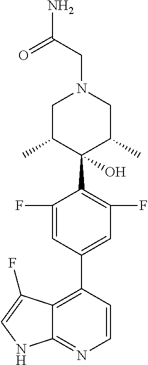 [SMILES: C[C@@H]1CN(CC(N)=O)C[C@H](C)[C@@]1(O)c1c(F)cc(-c2ccnc3[nH]cc(F)c23)cc1F]

### 2-((3R,4s,5S)-4-(2,6-difluoro-4-(3-fluoro-1H-pyrrolo[2,3-b]pyridin-4-yl)phenyl)-4-hydroxy-3,5-dimethylpiperidin-1-yl)acetamide

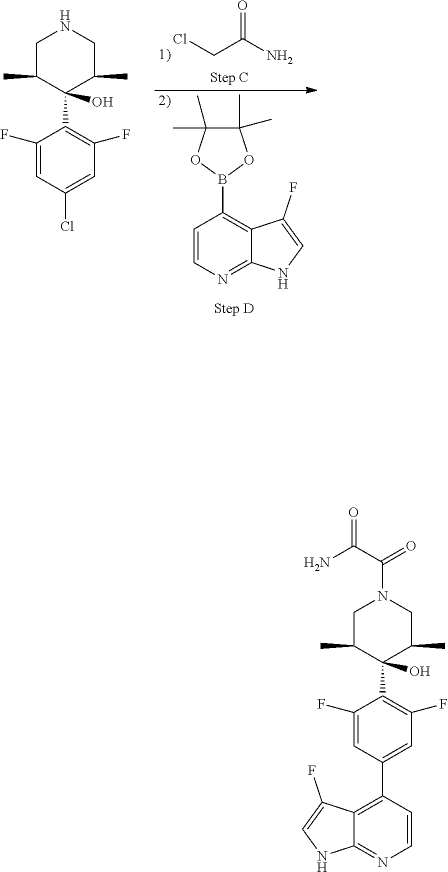

Step C: A vial was charged with (3S,4s,5R)-4-(4-chloro-2,6-difluorophenyl)-3,5-dimethylpiperidin-4-ol (0.080 g, 0.290 mmol, synthesis previously established), followed by 2-chloroacetamide (0.031 g, 0.332 mmol), potassium iodide (0.005 g, 0.029 mmol), and potassium carbonate (0.040 g, 0.290 mmol). Ethanol (3.0 ml) was added to the vial, and the reaction was stirred vigorously for 48 hours. Then additional 2-chloroacetamide (0.035 g, 0.27 mmol) was added, followed by an additional potassium carbonate (0.040 g, 0.290 mmol). The reaction was then sequentially heated by microwave irradiation to 60° C. for 0.5 hours, then 70° C. for 0.75 hours. The reaction was cooled, and poured into an aqueous saturated solution of sodium bicarbonate. The reaction was extracted into ethyl acetate. The organic phase was partitioned, and the solvent removed by rotary evaporation to give the title product as a crude solid, which was used without further purification. LC-MS (m/z [M+H]⁺): 333.1

Step D: A microwave vial was charged with 2-((3S,4s,5R)-4-(4-chloro-2,6-difluorophenyl)-4-hydroxy-3,5-dimethylpiperidin-1-yl)acetamide (0.058 g, 0.174 mmol), 3-fluoro-4-(4,4,5,5-tetramethyl-1,3,2-dioxaborolan-2-yl)-1H-pyrrolo[2,3-b]pyridine (0.05 g, 0.191 mmol), potassium phosphate (tribasic, K₃PO₄, 0.100 g, 0.471 mmol), and Chloro(2-dicyclohexylphosphino-2′,4′,6′-triisopropyl-1,1′-biphenyl)[2-(2′-amino-1,1′-biphenyl)]palladium(II) (Xphos Pd G2, 15.0 mg, 0.019 mmol). The vial was capped. Then, tetrahydrofuran (1.0 mL) was added, followed by water (1.0 ml). The content of the reaction vial was purged with nitrogen via bubbling through the solvent. The reaction was then heated in the microwave reactor at 100° C. for 45 min. The reaction was cooled to room temperature. A second portion of Chloro(2-dicyclohexylphosphino-2′,4′,6′-triisopropyl-1,1′-biphenyl)[2-(2′-amino-1,1′-biphenyl)]palladium(II) (Xphos Pd G2, 15.0 mg, 0.013 mmol) was added, followed by a second portion of 3-fluoro-4-(4,4,5,5-tetramethyl-1,3,2-dioxaborolan-2-yl)-1H-pyrrolo[2,3-b]pyridine (0.03 g, 0.114 mmol). The vial was sealed again, the solvent degassed by nitrogen bubbling, and the reaction was heated to 100° C. for a further 2 hours. The reaction was then cooled. The solvent was removed by rotary evaporation, and the crude mixtures was dissolved in methanol (2.0 ml). The reaction mixture was then filtered through a nylon syringe filter. Purification by reverse phase chromatography (5% to 100% acetonitrile in water with 0.1% trifluoroaceitic acid) gave the product. LC-MS (m/z [M+H]⁺): 433.2

### Compound 89

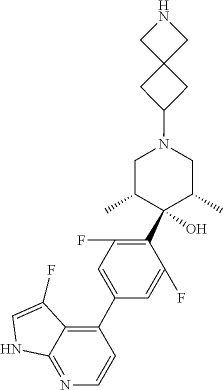 [SMILES: C[C@@H]1CN(C2CC3(CNC3)C2)C[C@H](C)[C@@]1(O)c1c(F)cc(-c2ccnc3[nH]cc(F)c23)cc1F]

### (3R,4s,5S)-4-(2,6-difluoro-4-(3-fluoro-1H-pyrrolo[2,3-b]pyridin-4-yl)phenyl)-3,5-dimethyl-1-(2-azaspiro[3.3]heptan-6-yl)piperidin-4-ol

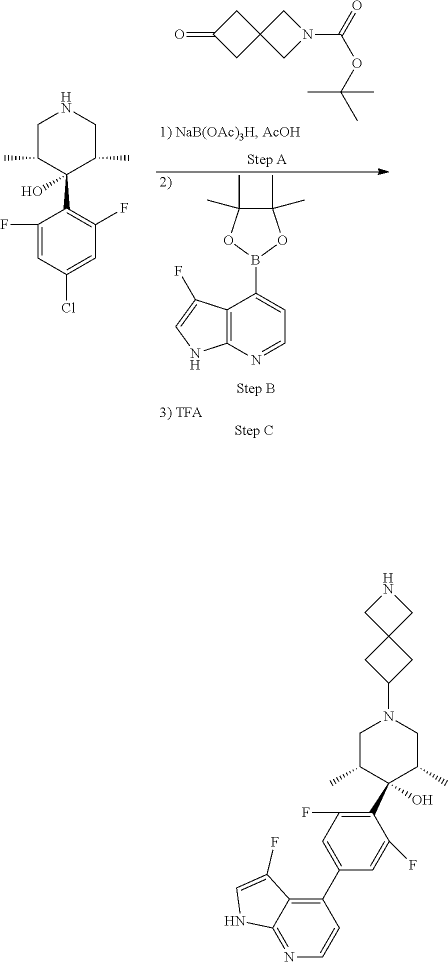

Step A: A vial was charged with (3S,4s,5R)-4-(4-chloro-2,6-difluorophenyl)-3,5-dimethylpiperidin-4-ol (0.100 g, 0.363 mmol) and tert-butyl 6-oxo-2-azaspiro[3.3]heptane-2-carboxylate (0.100 g, 0.473 mmol). Dichloromethane (4.0 mL) was added, followed be acetic acid (0.006 g, 0.10 mmol). The reaction was stirred for 10 minutes at room temperature, and then sodium triacetoxyborohydride (0.200 g, 0.944 mmol) was added. The reaction was allowed to stir a further 3 hours at room temperature. The reaction was then quenched by being poured into a saturated solution of aqueous sodium bicarbonate. The reaction mixture was extracted into ethyl acetate, the organic phase was taken, and the solvent removed by rotary evaporation. Purification by reverse phase chromatography gave the desired product. LC-MS (m/z [M+H]⁺): 471.2

Step B: A microwave vial was charged with tert-butyl 6-((3S,4s,5R)-4-(4-chloro-2,6-difluorophenyl)-4-hydroxy-3,5-dimethylpiperidin-1-yl)-2-azaspiro[3.3]heptane-2-carboxylate (0.050 g, 0.106 mmol), 3-fluoro-4-(4,4,5,5-tetramethyl-1,3,2-dioxaborolan-2-yl)-1H-pyrrolo[2,3-b]pyridine (0.05 g, 0.191 mmol), potassium phosphate (tribasic, K₃PO₄, 0.100 g, 0.471 mmol), and Chloro(2-dicyclohexylphosphino-2′,4′,6′-triisopropyl-1,1′-biphenyl)[2-(2′-amino-1,1′-biphenyl)]palladium(II) (Xphos Pd G2, 15.0 mg, 0.019 mmol). The vial was capped. Then, tetrahydrofuran (1.0 mL) was added, followed by water (1.0 ml). The content of the reaction vial was purged with nitrogen via bubbling through the solvent. The reaction was then heated in the microwave reactor at 100° C. for 45 min. The reaction was cooled to room temperature and poured into an aqueous solution of sodium bicarbonate. The mixture was extracted with ethyl acetate, the organic phases were partitioned, and the organic solvent removed by rotary evaporation to give the crude product which was used without any further purification. LC-MS (m/z [M-CO₂C(CH₃)₃—H]⁺): 471.2

Step C: A vial was charged with the crude material from step B. Dichloromethane (1.0 ml) was added to the vial, followed by trifluoroacetic acid (1.0 ml). The reaction was stirred for 0.5 hours at room temperature. Then the solvent and trifluoroacetic acid were removed by rotary evaporation. The crude residue was treated with saturated aqueous sodium bicarbonate and then extracted into ethyl acetate. The organic phase was taken, and the solvent was removed by rotary evaporation. The crude residue was dissolved in methanol (2.0 ml), filtered through a nylon syringe filter, and purified by reverse phase HPLC to provide the title compound as the trifluoroacetate salt. ¹H NMR (400 MHz, DMSO-d₆) δ 11.80 (s, 1H), 9.88 (s, 1H), 8.65 (s, 2H), 8.35 (d, J=4.9 Hz, 1H), 7.61 (s, 1H), 7.41 (d, J=12.9 Hz, 2H), 7.27 (d, J=4.9 Hz, 1H), 5.54 (s, 1H), 4.05-4.00 (m, 2H), 3.97-3.92 (m, 2H), 3.68-3.58 (m, 1H), 3.18 (d, J=11.7 Hz, 2H), 2.81-2.71 (m, 1H), 2.68-2.52 (m, 4H), 0.85 (d, J=6.7 Hz, 6H). LC-MS (m/z [M+H]⁺): 471.3.

### Compounds 90-91

Compounds 90-91 were prepared using an analogous procedure to that of Compound 89, and are shown in Table 15.

TABLE 15

| | | | | LCMS |
|---|---|---|---|---|
| Compound | Structure | IUPAC name | ¹H NMR | m/z |
| 90 | 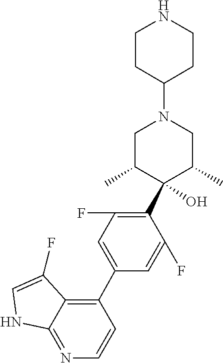 [SMILES: C[C@@H]1CN(C2CCNCC2)C[C@H](C)[C@@]1(O)c1c(F)cc(-c2ccnc3[nH]cc(F)c23)cc1F] | (3R,4s,5S)-4- (2,6-difluoro-4- (3-fluoro-1H- pyrrolo[2,3- b]pyridin-4- yl)phenyl)-3,5- dimethyl-[1,4′- bipiperidin]-4-ol | ¹H NMR (400 MHz, DMSO-d₆) δ 11.80 (s, 1H), 9.98 (s, 1H), 8.75 (s, 1H), 8.50 (s, 1H), 8.36 (d, J = 5.0 Hz, 1H), 7.61 (s, 1H), 7.41 (d, J = 13.2 Hz, 2H), 7.27 (d, J = 4.9 Hz, 1H), 5.56 (s, 1H), 3.53-3.43 (m, 5H), 2.96 (dd, J = 26.3, 12.5 Hz, 4H), 2.79-2.68 (m, 2H), 2.33 (d, J = 12.0 Hz, 2H), 1.91-1.75 (m, 2H), 0.86 (d, J = 6.7 Hz, 6H). | [M + H]⁺: 459.3 |
| 91 | 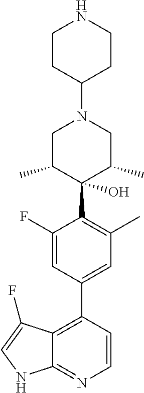 [SMILES: Cc1cc(-c2ccnc3[nH]cc(F)c23)cc(F)c1[C@@]1(O)[C@H](C)CN(C2CCNCC2)C[C@@H]1C] | (3S,4s,5R)-4-(2- fluoro-4-(3- fluoro-1H- pyrrolo[2,3- b]pyridin-4-yl)- 6- methylphenyl)- 3,5-dimethyl- [1,4′- bipiperidin]-4-ol | ¹H NMR (400 MHz, Methanol-d₄) δ 8.29 (d, J = 5.0 Hz, 1H), 7.36 (s, 1H), 7.31 (s, 2H), 7.28 (s, 1H), 7.20 (d, J = 5.1 Hz, 1H), 3.68-3.56 (m, 4H), 3.40-3.33 (m, 2H), 3.28- 3.20 (m, 1H), 3.13 (t, J = 13.3 Hz, 2H), 3.07- 2.92 (m, 2H), 2.77 (s, 3H), 2.49 (d, J = 13.4 Hz, 2H), 2.12-1.96 (m, 2H), 0.93 (d, J = 6.9 Hz, 6H). | [M + H]⁺: 455.3 |

### Compound 92

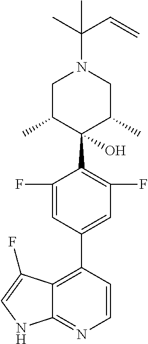 [SMILES: C=CC(C)(C)N1C[C@@H](C)[C@](O)(c2c(F)cc(-c3ccnc4[nH]cc(F)c34)cc2F)[C@@H](C)C1]

### (3S,4s,5R)-4-(2,6-difluoro-4-(3-fluoro-1H-pyrrolo[2,3-b]pyridin-4-yl)phenyl)-3,5-dimethyl-1-(2-methylbut-3-en-2-yl)piperidin-4-ol

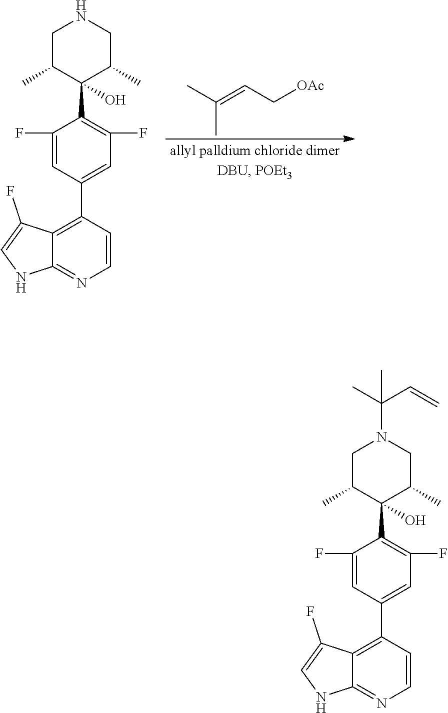

Step A: A vial was charged with allylpalladium(II) chloride dimer (2.5 mg, 0.007 mmol). The vial was sealed, evacuated, and backfilled with nitrogen. Tetrahydrofuran was added (0.5 mL), followed by triethylphosphite (3.0 mg, 0.018 mmol). The reaction was stirred for 5 minutes. Then 3-methylbut-2-en-1-yl acetate (17.1 mg, 0.133 mmol was added), followed by a solution of (3S,4s,5R)-4-(2,6-difluoro-4-(3-fluoro-1H-pyrrolo[2,3-b]pyridin-4-yl)phenyl)-3,5-dimethylpiperidin-4-ol (0.050 g, 0.133 mmol) in methanol (2.0 ml). Finally 2,3,4,6,7,8,9,10-octahydropyrimido[1,2-a]azepine (0.020 g, 0.133 mmol, DBU) was added. The reaction was stirred for 2 hours at room temperature. The reaction was then poured into aqueous sodium bicarbonate and extracted into ethyl acetate. The organic phase was partitioned, collected, and the solvent removed by rotary evaporation. Purification by reverse phase chromatography (5% to 100% acetonitrile in water, with 0.1% formic acid) gave the title product. ¹H NMR (600 MHz, Methanol-d₄) δ 8.30 (d, J=5.0 Hz, 1H), 7.34 (d, J=2.0 Hz, 1H), 7.28 (dt, J=12.5, 2.9 Hz, 2H), 7.22 (dd, J=5.0, 0.8 Hz, 1H), 5.99 (dd, J=17.6, 10.9 Hz, 1H), 5.16-5.09 (m, 2H), 2.70 (dd, J=11.1, 3.6 Hz, 2H), 2.61 (dqd, J=10.6, 6.9, 3.5 Hz, 2H), 2.50 (td, J=11.2, 2.6 Hz, 2H), 1.23 (s, 6H), 0.84 (d, J=6.9 Hz, 6H). LC-MS (m/z [M+H]⁺): 444.3

### Compound 93

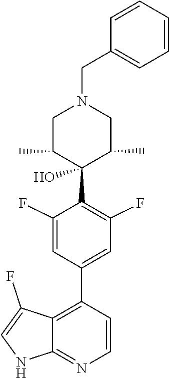 [SMILES: C[C@@H]1CN(Cc2ccccc2)C[C@H](C)[C@@]1(O)c1c(F)cc(-c2ccnc3[nH]cc(F)c23)cc1F]

### (3S,4s,5R)-1-benzyl-4-(2,6-difluoro-4-(3-fluoro-1H-pyrrolo[2,3-b]pyridin-4-yl)phenyl)-3,5-dimethylpiperidin-4-ol

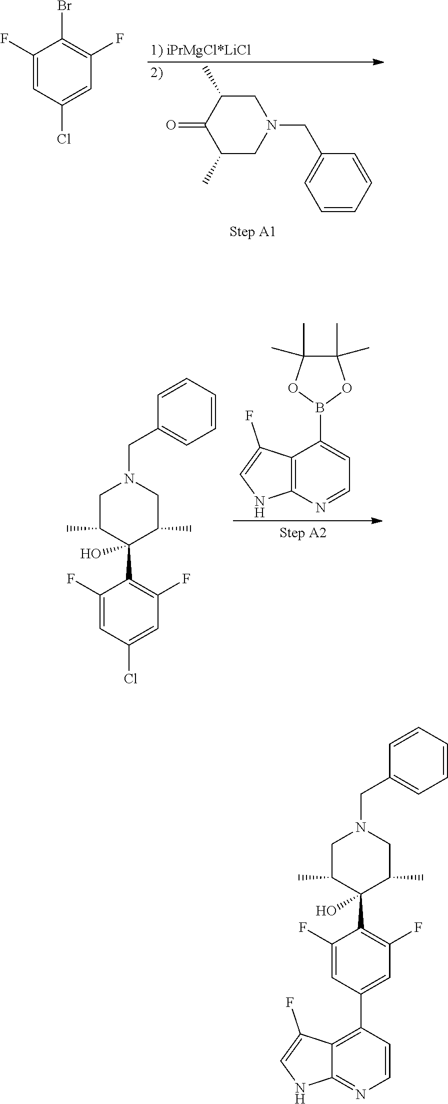

Step A1: A vial was charged with 2-bromo-5-chloro-1,3-difluorobenzene (0.686 g, 3.02 mmol). The vial was sealed, evacuated, and backfilled with nitrogen. Then tetrahydrofuran (1.0 mL) was added, and the vial was cooled to 0° C. Then isopropyl magnesium chloride lithium chloride in tetrahydrofuran (2.30 mL, 3.02 mmol, 1.3 M) was added via syringe. The reaction was stirred for 1 hour and the temperature was allowed to raise to room temperature. In a separate microwave vial was added (3S,5R)-1-benzyl-3,5-dimethylpiperidin-4-one (0.655 g, 3.02 mmol). The vial was sealed and tetrahydrofuran (1.0 mL) was added. The contents of the first vial containing the magnesium reagent were then removed by syringe and added to the contents of the second vial, containing the ketone. The reaction was then heated to 60° C. by microwave irradiation for 1.5 hours. The reaction was cooled, and poured into an aqueous saturated solution of sodium bicarbonate. The mixture was extracted with ethyl acetate (2×), the organic phases were partitioned, and the organic layers were combined. The solvent was removed by rotary evaporation to give the crude product. Purification by reverse phase chromatography (5% to 100% acetonitrile in water with 0.1% formic acid) gave the title product. ¹H NMR (400 MHz, Chloroform-d) δ 7.45-7.29 (m, 4H), 7.27 (d, J=7.3 Hz, 1H), 7.01-6.80 (m, 2H), 3.56 (s, 2H), 2.69 (d, J=14.2 Hz, 1H), 2.59 (dq, J=8.0, 4.4 Hz, 4H), 2.25 (td, J=12.2, 2.9 Hz, 2H), 0.76 (d, J=6.6 Hz, 6H). LC-MS (m/z [M+H]⁺): 366.1

Step A2: A microwave vial was charged with ((3S,4s,5R)-1-benzyl-4-(4-chloro-2,6-difluorophenyl)-3,5-dimethylpiperidin-4-ol (0.050 g, 0.137 mmol), 3-fluoro-4-(4,4,5,5-tetramethyl-1,3,2-dioxaborolan-2-yl)-1H-pyrrolo[2,3-b]pyridine (0.050 g, 0.191 mmol), potassium phosphate (tribasic, K₃PO₄, 0.100 g, 0.471 mmol), and Chloro(2-dicyclohexylphosphino-2′,4′,6′-triisopropyl-1,1′-biphenyl)[2-(2′-amino-1,1′-biphenyl)]palladium(II) (Xphos Pd G2, 15.0 mg, 0.019 mmol). The vial was sealed. Then, tetrahydrofuran (1.0 mL) was added, followed by water (1.0 ml). The content of the reaction vial was purged with nitrogen via bubbling through the solvent. The reaction was then heated in the microwave reactor at 100° C. for 45 min. The reaction was cooled to room temperature. The reaction was poured into saturated aqueous sodium bicarbonate and extracted into ethyl acetate (2×). The organic phases were partitioned, combined, and the solvent removed by rotary evaporation. Purification by reverse-phase HPLC to provided the title compound. ¹H NMR (400 MHz, DMSO-d₆) δ 11.79 (s, 1H), 9.81-9.73 (m, 2H), 8.35 (d, J=4.9 Hz, 1H), 7.62-7.56 (m, 3H), 7.51 (dd, J=4.9, 1.9 Hz, 3H), 7.44-7.35 (m, 1H), 7.26 (d, J=4.9 Hz, 1H), 5.55 (s, 1H), 4.38 (d, J=4.8 Hz, 2H), 3.18 (d, J=11.2 Hz, 2H), 3.12-2.97 (m, 2H), 2.75-2.65 (m, 2H), 0.82 (d, J=6.8 Hz, 6H). LC-MS (m/z [M+H]⁺): 466.3

### Compound 94

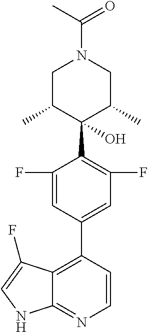 [SMILES: CC(=O)N1C[C@@H](C)[C@](O)(c2c(F)cc(-c3ccnc4[nH]cc(F)c34)cc2F)[C@@H](C)C1]

### 1-((3S,4s,5R)-4-(2,6-difluoro-4-(3-fluoro-1H-pyrrolo[2,3-b]pyridin-4-yl)phenyl)-4-hydroxy-3,5-dimethylpiperidin-1-yl)ethan-1-one

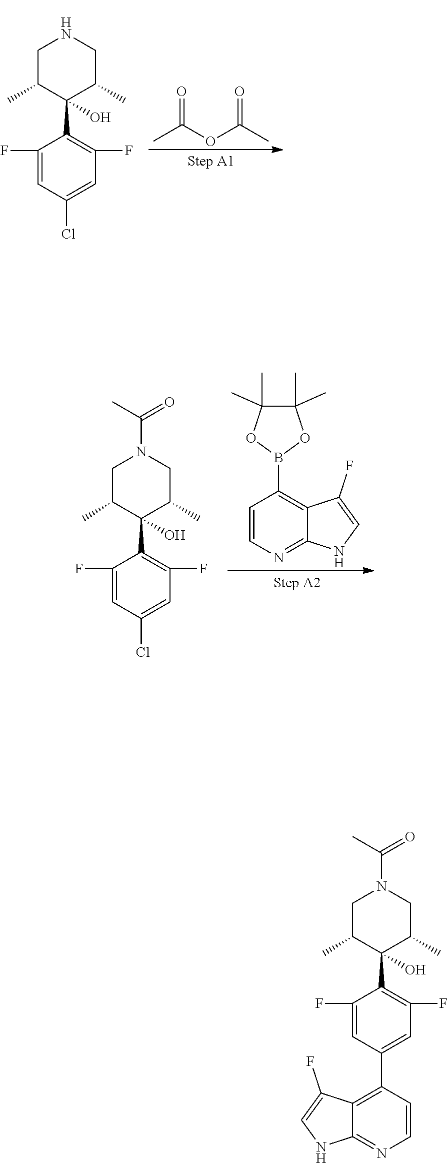

Step A1: To a solution of (3S,4s,5R)-4-(4-chloro-2,6-difluorophenyl)-3,5-dimethylpiperidin-4-ol (0.045 g, 0.163 mmol) in tetrahydrofuran (2.0 ml) was added acetic anhydride (0.108 g, 1.06 mmol) followed by diisopropylethylamine (0.074 g, 0.573 mmol). The reaction was stirred for 0.5 hours at room temperature. It was then poured into a saturated aqueous solution of sodium bicarbonate, and extracted into ethyl acetate. The organic phase was partitioned, and the solvent removed by rotary evaporation to give the crude product, which was used without further purification. LC-MS (m/z [M+H]⁺): 318.1

Step A2: A microwave vial was charged with 1-((3S,4s,5R)-4-(4-chloro-2,6-difluorophenyl)-4-hydroxy-3,5-dimethylpiperidin-1-yl)ethan-1-one (From step A1), 3-fluoro-4-(4,4,5,5-tetramethyl-1,3,2-dioxaborolan-2-yl)-1H-pyrrolo[2,3-b]pyridine (0.050 g, 0.191 mmol), potassium phosphate (tribasic, K₃PO₄, 0.100 g, 0.471 mmol), and Chloro(2-dicyclohexylphosphino-2′,4′,6′-triisopropyl-1,1′-biphenyl)[2-(2′-amino-1,1′-biphenyl)]palladium(II) (Xphos Pd G2, 15.0 mg, 0.019 mmol). The vial was sealed. Then, tetrahydrofuran (1.0 mL) was added, followed by water (1.0 ml). The content of the reaction vial was purged with nitrogen via bubbling through the solvent. The reaction was then heated in the microwave reactor at 100° C. for 45 min. The reaction was cooled to room temperature. The reaction was poured into saturated aqueous sodium bicarbonate and extracted into ethyl acetate (2×). The organic phases were partitioned, combined, and the solvent removed by rotary evaporation. Purification by reverse-phase HPLC to provided the title compound. ¹H NMR (400 MHz, DMSO-d₆) δ 11.76 (s, 1H), 8.34 (d, J=4.9 Hz, 1H), 7.78-7.54 (m, 1H), 7.35 (dd, J=12.3, 2.6 Hz, 2H), 7.26 (d, J=4.9 Hz, 1H), 4.99 (s, 1H), 4.19 (d, J=10.3 Hz, 1H), 3.60-3.47 (m, 1H), 3.16 (t, J=12.4 Hz, 1H), 2.64 (t, J=12.1 Hz, 2H), 2.39-2.28 (m, 1H), 2.27-2.17 (m, 1H), 2.05 (s, 3H), 0.80 (t, J=7.4 Hz, 6H). LC-MS (m/z [M+H]⁺): 418.2

### Compound 95

Compound 95 was prepared using an analogous procedure to that of Compound 94 and is shown in Table 16.

TABLE 16

| | | | | LCMS |
|---|---|---|---|---|
| Compound | Structure | IUPAC name | ¹H NMR | m/z |
| 95 | 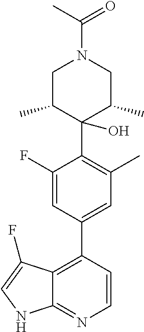 [SMILES: CC(=O)N1C[C@@H](C)C(O)(c2c(C)cc(-c3ccnc4[nH]cc(F)c34)cc2F)[C@@H](C)C1] | 1-((3S,5R)-4-(2- fluoro-4-(3-fluoro- 1H-pyrrolo[2,3- b]pyridin-4-yl)-6- methylphenyl)-4- hydroxy-3,5- dimethylpiperidin- 1-yl)ethan-1-one | ¹H NMR (400 MHz, DMSO-d₆) δ 11.67 (s, 1H), 8.30 (d, J = 4.9 Hz, 1H), 7.53 (s, 1H), 7.30- 7.15 (m, 3H), 4.56 (s, 1H), 4.18 (d, J = 12.7 Hz, 1H), 3.53 (d, J = 13.2 Hz, 1H), 3.16 (t, J = 12.4 Hz, 1H), 2.71 (s, 3H), 2.69-2.55 (m, 1H), 2.46-2.40 (m, 1H), 2.39-2.28 (m, 1H), 2.04 (s, 3H), 0.73 (t, J = 7.3 Hz, 6H). | [M + H]⁺: 414.2 |

### Compound 318

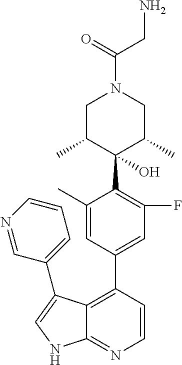 [SMILES: Cc1cc(-c2ccnc3[nH]cc(-c4cccnc4)c23)cc(F)c1[C@@]1(O)[C@H](C)CN(C(=O)CN)C[C@@H]1C]

### 2-amino-1-((3S,4s,5R)-4-(2-fluoro-6-methyl-4-(3-(pyridin-3-yl)-1H-pyrrolo[2,3-b]pyridin-4-yl)phenyl)-4-hydroxy-3,5-dimethylpiperidin-1-yl)ethan-1-one

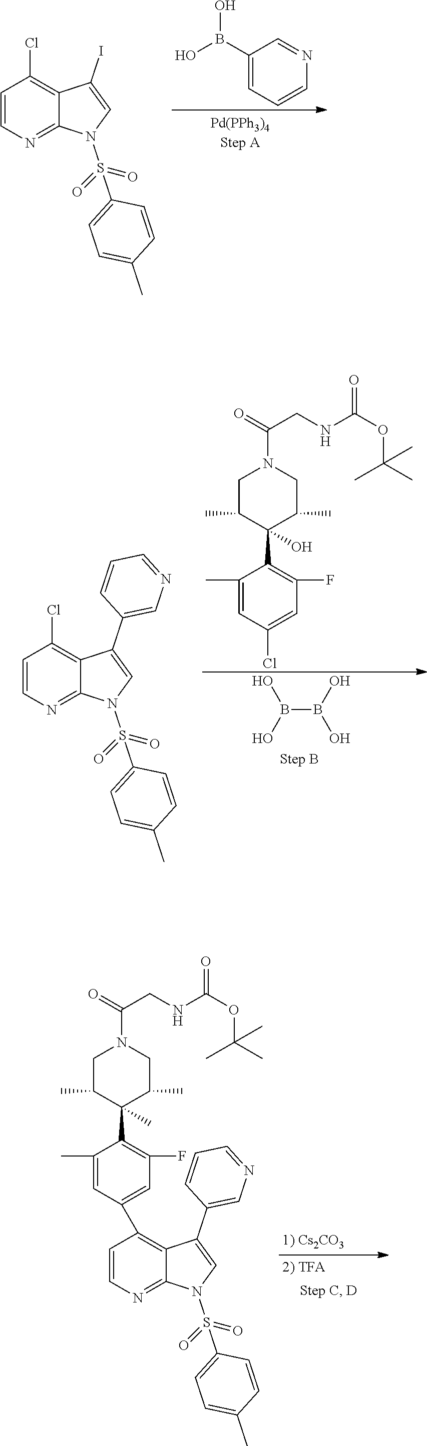

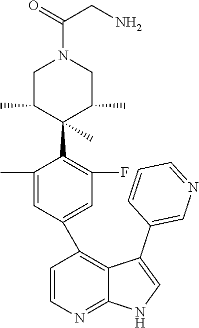 [SMILES: Cc1cc(-c2ccnc3[nH]cc(-c4cccnc4)c23)cc(F)c1[C@@]1(C)[C@H](C)CN(C(=O)CN)C[C@@H]1C]

Step A: An oven-dried 20 mL microwave vial containing a stirbar was charged with 4-chloro-3-iodo-1-tosyl-1H-pyrrolo[2,3-b]pyridine (0.300 g, 0.693 mmol), pyridin-3-ylboronic acid (0.128 g, 1.04 mmol), potassium carbonate (0.287 g, 2.08 mmol) and tetrakis(triphenylphosphine)palladium(0) (0.080 g, 0.069 mmol). The vial was sealed, evacuated, and backfilled with nitrogen gas. 1,4-Dioxane (8.0 ml) followed by water (2.0 ml) was added, and the solution was purged with nitrogen gas bubbling for 5 minutes. The reaction was then heated to 150° C. for 30 minutes by microwave irradiation. The vial was then cooled, washed with a saturated solution of aqueous sodium bicarbonate and extracted into ethyl acetate. The organic phase was taken, and the solvent removed by rotary evaporation to give the crude product. Purification by reverse phase medium pressure liquid chromatography (5% to 100% acetonitrile in water, containing 0.1% trifluoroacetic acid) gave the desired intermediate 4-chloro-3-(pyridin-3-yl)-1-tosyl-1H-pyrrolo[2,3-b]pyridine. LC-MS m/z: 384.0 ([M+H]⁺.

Step B: An oven-dried 5 mL microwave vial containing a stirbar was charged with tert-butyl (2-((3S,4s,5R)-4-(4-chloro-2-fluoro-6-methylphenyl)-4-hydroxy-3,5-dimethylpiperidin-1-yl)-2-oxoethyl)carbamate (0.050 g, 0.117 mmol), hypodiboric acid (0.031 g, 0.350 mmol), potassium acetate (0.034 g, 3.0 mmol), di-tert-butyl(2′,4′,6′-triisopropyl-[1,1′-biphenyl]-2-yl)phosphane (0.0025 g, 0.006 mmol), and chloro(2-dicyclohexylphosphino-2′,4′,6′-triisopropyl-1,1′-biphenyl)[2-(2′-amino-1,1′-biphenyl)]palladium(II) (0.005 g, 0.006 mmol, Xphos Pd G2). The vial was sealed, evacuated, and backfilled with argon. Ethanol (3.0 mL, previously purged with argon gas), was then added to the vial. The reaction was than heated to 80° C. for 30 minutes via microwave irradiation. The vial was cooled, and an argon-purged solution containing 4-chloro-3-(pyridin-3-yl)-1-tosyl-1H-pyrrolo[2,3-b]pyridine (0.049 g, 0.128 mmol), potassium carbonate (0.048 g, 0.350 mmol), ethanol (1.0 mL) and water (1.0 ml) was added to the sealed via by syringe. The reaction was then heated to 100° C. for 45 minutes by microwave irradiation. It was then cooled, washed with saturated aqueous sodium bicarbonate and extracted into ethyl acetate. The organic phase was taken and the solvent removed by rotary evaporation. Purification by silica gel chromatography (0% to 100% ethyl acetate in hexanes) gave the desired intermediate tert-butyl (2-((3S,4s,5R)-4-(2-fluoro-6-methyl-4-(3-(pyridin-3-yl)-1-tosyl-1H-pyrrolo[2,3-b]pyridin-4-yl)phenyl)-4-hydroxy-3,5-dimethylpiperidin-1-yl)-2-oxoethyl)carbamate. LC-MS m/z: 742.3 ([M+H]⁺.

Step C: An oven-dried vial containing a stirbar was charged with tert-butyl (2-((3S,4s,5R)-4-(2-fluoro-6-methyl-4-(3-(pyridin-3-yl)-1H-pyrrolo[2,3-b]pyridin-4-yl)phenyl)-4-hydroxy-3,5-dimethylpiperidin-1-yl)-2-oxoethyl)carbamate (0.025 g, 0.034 mmol). Tetrahydrofuran (1.0 mL) and methanol (0.5 ml) were added, followed by cesium carbonate (0.042 g, 0.129 mmol) and water (0.60 mg, 0.034 mmol). The reaction was stirred for 2 hours. It was then extracted into ethyl acetate, and the organic solvent was removed by rotary evaporation to give the desired intermediate tert-butyl (2-((3S,4s,5R)-4-(2-fluoro-6-methyl-4-(3-(pyridin-3-yl)-1H-pyrrolo[2,3-b]pyridin-4-yl)phenyl)-4-hydroxy-3,5-dimethylpiperidin-1-yl)-2-oxoethyl)carbamate. LC-MS m/z: 588.3 ([M+H]⁺.

Step D: tert-butyl (2-((3S,4s,5R)-4-(2-fluoro-6-methyl-4-(3-(pyridin-3-yl)-1H-pyrrolo[2,3-b]pyridin-4-yl)phenyl)-4-hydroxy-3,5-dimethylpiperidin-1-yl)-2-oxoethyl)carbamate (From Step C) was dissolved in dichloromethane (1.0 mL). Trifluoroacetic acid (1.0 ml) was added, and the reaction was stirred at room temperature for 30 minutes. The solvent and trifluoroacetaic acid were then removed by rotary evaporation. Purification by reverse phase high pressure liquid chromatography (5% to 100% acetonitrile in water with 0.1% trifluoroacetic acid) gave the title product. ¹H NMR (400 MHz, DMSO-d₆) δ 12.28 (s, 1H), 8.36 (d, J=4.9 Hz, 1H), 8.32 (d, J=4.8 Hz, 1H), 8.30-8.27 (m, 1H), 8.03-7.96 (m, 4H), 7.80 (d, J=2.7 Hz, 1H), 7.41-7.37 (m, 1H), 7.15 (d, J=4.8 Hz, 1H), 6.80 (d, J=14.2 Hz, 1H), 6.70 (d, J=1.9 Hz, 1H), 4.53 (s, 1H), 4.27 (s, 1H), 4.21-4.13 (m, 1H), 4.10-4.01 (m, 1H), 3.90-3.81 (m, 1H), 3.43-3.36 (m, 1H), 3.18-3.16 (m, 1H), 2.79-2.71 (m, 1H), 2.69-2.66 (m, 1H), 2.35 (s, 3H), 0.74 (dd, J=6.9, 3.4 Hz, 6H). LC-MS m/z: 488.1 [M+H]⁺.

### Synthesis of Compounds of General Formula A5, A6, A7, and A8

Compounds of general formula A5, A6, A7 and A8 can be prepared as illustrated in Scheme 5 below.

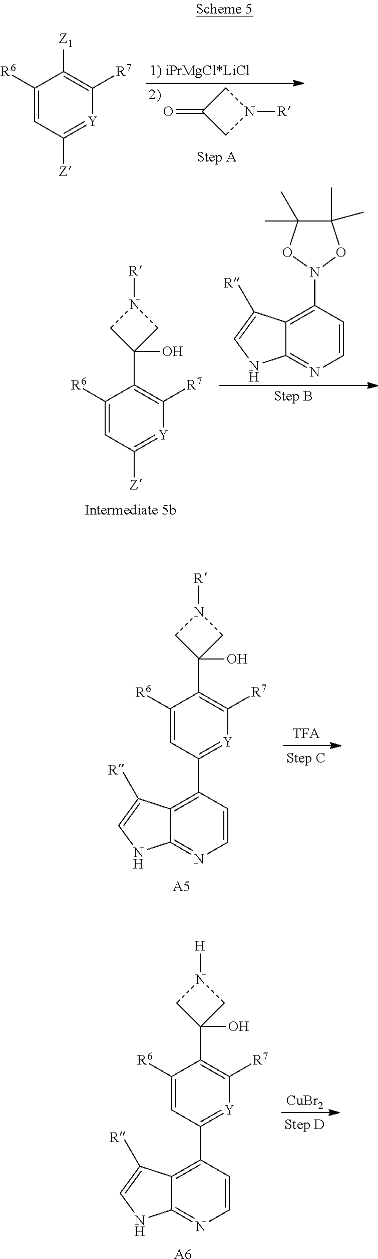

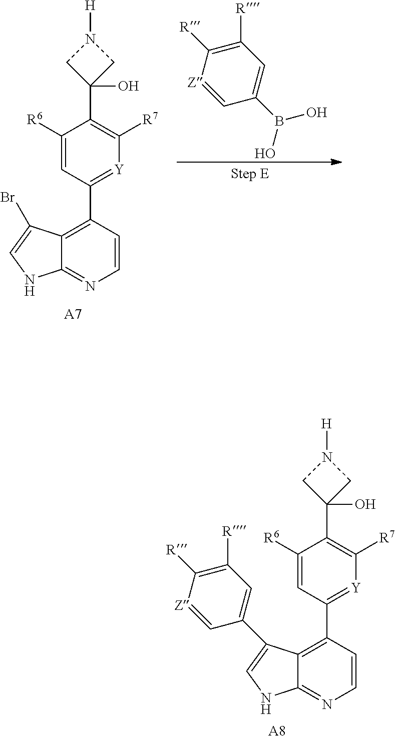

### Synthesis of Intermediates and Compounds of Scheme 5

### Intermediate 5b (Scheme 5): tert-butyl 4-(4-bromo-2-chlorophenyl)-4-hydroxypiperidine-1-carboxylate

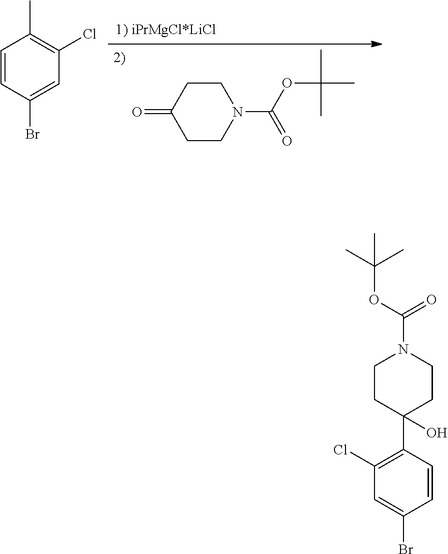

Step A: An oven-dried flask was charged with 4-bromo-2-chloro-1-iodobenzene (0.317 g, 1.00 mmol) and tetrahydrofuran (1.6 mL). The resulting solution was cooled down to 0-5° C. using an ice-water bath. Then, an iso-propyl magnesium chloride lithium chloride complex solution (1.3 M in tetrahydrofuran) (7.0 mL, 1.30 mmol) was added drop wise. The cooling bath was removed, and the resulting solution was stirred at 23° C. for 1.0 h. Then, a solution containing 1-Boc-4-piperidone (0.239 g, 1.200 mmol) in tetrahydrofuran (0.40 mL) was added slowly, and the resulting reaction mixture was allowed to warm up progressively to room temperature and was stirred at 23° C. for 23 hours. The reaction mixture was cooled by the mean of an ice-water bath, and the reaction was quenched upon addition of 1.5 mL of saturated aqueous NH₄Cl. The reaction mixture was partitioned between 10 mL of water and 10 mL of tert-butyl methyl ether. The layers were separated; the aqueous layer was extracted with two 10-mL portions of tert-butyl methyl ether. The combined organic layer was washed with 20 mL of brine, was then dried (sodium sulfate), and was then concentrated under diminished pressure. The residue was applied to a silica gel column; eluting with 100:0→30:70 hexanes-ethyl acetate provided the title compound. ¹H NMR (400 MHz, Chloroform-d) δ 7.54 (dd, J=1.9, 0.4 Hz, 1H), 7.49-7.35 (m, 2H), 4.05 (d, J=13.2 Hz, 2H), 3.33-3.18 (m, 2H), 2.27 (td, J=13.2, 4.9 Hz, 2H), 1.97-1.83 (m, 2H), 1.48 (s, 9H). LC-MS (m/z [M−OC(CH₃)₃+H)]⁺): 317.9

### Intermediates 5b-2 to 5b-11 (Scheme 5)

Intermediates 5b-2 to 5b-11 were prepared using an analogous procedure to that of Intermediate 5b-1, and are shown in Table 17.

TABLE 17

| Compound | Structure | IUPAC name | LCMS m/z |
|---|---|---|---|
| 5b-2 | 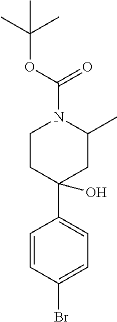 [SMILES: CC1CC(O)(c2ccc(Br)cc2)CCN1C(=O)OC(C)(C)C] | tert-butyl 4-(4-chlorophenyl)-4- hydroxy-2-methylpiperidine-1- carboxylate | [M − CO₂C(CH₃)₃ + H]⁺ 270.0 |
| 5b-3 | 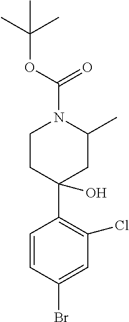 [SMILES: CC1CC(O)(c2ccc(Br)cc2Cl)CCN1C(=O)OC(C)(C)C] | tert-butyl 4-(4-bromo-2- chlorophenyl)-4-hydroxy-2- methylpiperidine-1-carboxylate | [M − OC(CH₃)₃ + H]⁺ 331.9 |
| 5b-4 | 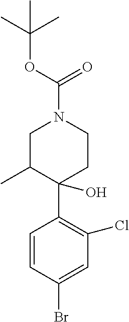 [SMILES: CC1CN(C(=O)OC(C)(C)C)CCC1(O)c1ccc(Br)cc1Cl] | tert-butyl 4-(4-bromo-2- chlorophenyl)-4-hydroxy-3- methylpiperidine-1-carboxylate | [M − OC(CH₃)₃ + H]⁺ 332.0 |
| 5b-5 | 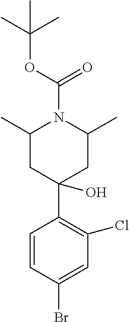 [SMILES: CC1CC(O)(c2ccc(Br)cc2Cl)CC(C)N1C(=O)OC(C)(C)C] | tert-butyl 4-(4-bromo-2- chlorophenyl)-4-hydroxy-2,6- dimethylpiperidine-1-carboxylate | [M − C(CH₃)₃ + 2H]⁺ 364.0 |
| 5b-6 | 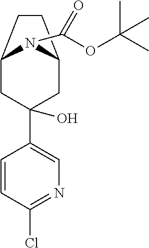 [SMILES: CC(C)(C)OC(=O)N1[C@@H]2CC[C@H]1CC(O)(c1ccc(Cl)nc1)C2] | tert-butyl (1R,5S)-3-(6- chloropyridin-3-yl)-3-hydroxy-8- azabicyclo[3.2.1]octane-8- carboxylate | [M + H]⁺ 338.9 |
| 5b-7 | 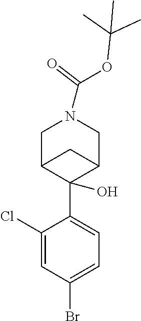 [SMILES: CC(C)(C)OC(=O)N1CC2CC(C1)C2(O)c1ccc(Br)cc1Cl] | tert-butyl 6-(4-bromo-2- chlorophenyl)-6-hydroxy-3- azabicyclo[3.1.1]heptane-3- carboxylate | [M − OC(CH₃)₃ + H]⁺ 330.3 |
| 5b-8 | 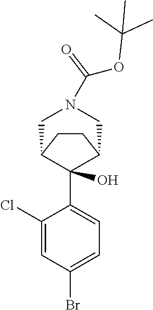 [SMILES: CC(C)(C)OC(=O)N1C[C@H]2CC[C@@H](C1)[C@@]2(O)c1ccc(Br)cc1Cl] | tert-butyl (1R,5S,8s)-8-(4-bromo- 2-chlorophenyl)-8-hydroxy-3- azabicyclo[3.2.1]octane-3- carboxylate | [M − C(CH₃)₃ + 2H]⁺ 359.7 |
| 5b-9 | 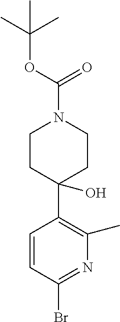 [SMILES: Cc1nc(Br)ccc1C1(O)CCN(C(=O)OC(C)(C)C)CC1] | tert-butyl 4-(6-bromo-2- methylpyridin-3-yl)-4- hydroxypiperidine-1-carboxylate | [M + H]⁺ 373.1 |
| 5b-10 | 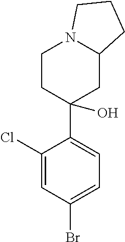 [SMILES: OC1(c2ccc(Br)cc2Cl)CCN2CCCC2C1] | 7-(4-bromo-2- chlorophenyl)octahydroindolizin- 7-ol | [M + H]⁺ 332.0 |
| 5b-11 | 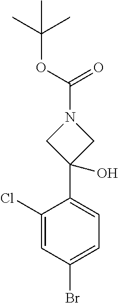 [SMILES: CC(C)(C)OC(=O)N1CC(O)(c2ccc(Br)cc2Cl)C1] | tert-butyl 3-(4-bromo-2- chlorophenyl)-3-hydroxyazetidine- 1-carboxylate | [M − (CH₃)₃ + 2H]⁺ 307.9 |

### Compound 96

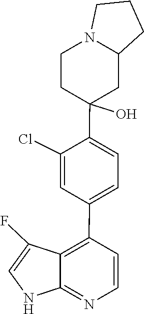 [SMILES: OC1(c2ccc(-c3ccnc4[nH]cc(F)c34)cc2Cl)CCN2CCCC2C1]

### 7-(2-chloro-4-(3-fluoro-1H-pyrrolo[2,3-b]pyridin-4-yl)phenyl)octahydroindolizin-7-ol

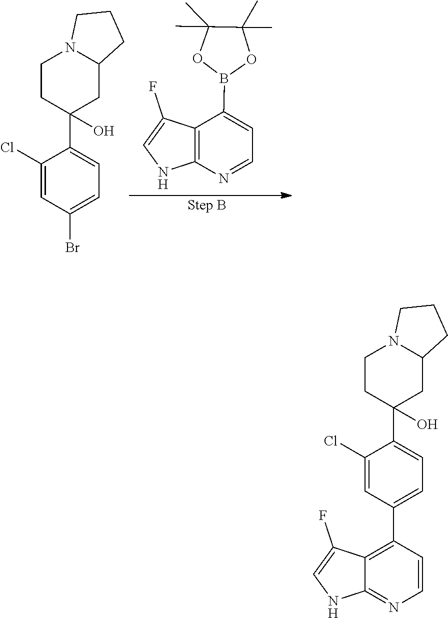 [SMILES: OC1(c2ccc(Br)cc2Cl)CCN2CCCC2C1 ; CC1(C)OB(c2ccnc3[nH]cc(F)c23)OC1(C)C ; OC1(c2ccc(-c3ccnc4[nH]cc(F)c34)cc2Cl)CCN2CCCC2C1]

Step B: A microwave vial was charged with 7-(4-bromo-2-chlorophenyl)octahydroindolizin-7-ol (0.053 g, 0.160 mmol), 3-fluoro-4-(4,4,5,5-tetramethyl-1,3,2-dioxaborolan-2-yl)-1H-pyrrolo[2,3-b]pyridine (0.046 g, 0.176 mmol), and tetrakis(triphenylphosphine)palladium(0) (9.26 mg, 8.01 μmol). The vial was sealed. Its contents were purged with nitrogen twice. Then, dioxane (0.480 ml) was added, followed by 2 M aqueous potassium carbonate (0.120 ml, 0.240 mmol). The content of the reaction vial was purged with nitrogen twice, and was then heated in the microwave reactor at 120° C. for 20 min. The reaction mixture was partitioned between 10 mL of water and 10 mL of ethyl acetate. The layers were separated; the aqueous layer was extracted with three 10 mL portions of ethyl acetate. The combined organic layer was washed with 10 mL of brine, was then dried over anhydrous sodium sulfate, filtered, and was then concentrated under diminished pressure. Purification by reverse-phase HPLC to provided the title compound. ¹H NMR (400 MHz, DMSO-d₆) δ 11.71 (d, J=2.9 Hz, 1H), 8.33 (d, J=4.9 Hz, 1H), 8.15 (s, 1H), 8.05-7.81 (m, 1H), 7.65 (q, J=3.0 Hz, 2H), 7.56 (t, J=2.4 Hz, 1H), 7.20 (d, J=5.0 Hz, 1H), 5.23 (s, 1H), 3.40-3.10 (m, 3H) 3.04 (t, J=8.3 Hz, 1H), 3.00-2.92 (m, 1H), 2.58-2.52 (m, 1H), 2.68 (td, J=12.5, 4.4 Hz, 1H), 2.35 (t, J=11.8 Hz, 1H), 2.21 (d, J=9.0 Hz, 1H), 1.79 (d, J=13.0 Hz, 1H), 1.75-1.65 (m, 1H), 1.60 (d, J=12.9 Hz, 1H), 1.35 (dt, J=19.9, 11.0 Hz, 1H). LC-MS (m/z [M+H)]⁺): 386.2

### Compound 97

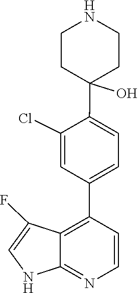 [SMILES: OC1(c2ccc(-c3ccnc4[nH]cc(F)c34)cc2Cl)CCNCC1]

### 4-(2-chloro-4-(3-fluoro-1H-pyrrolo[2,3-b]pyridin-4-yl)phenyl)piperidin-4-ol

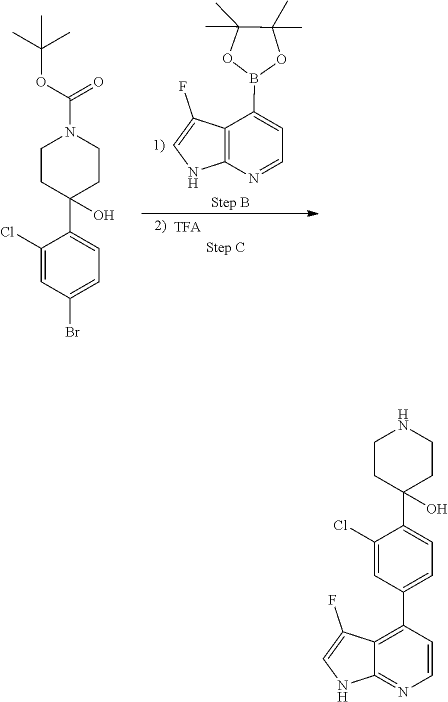 [SMILES: CC(C)(C)OC(=O)N1CCC(O)(c2ccc(Br)cc2Cl)CC1 ; CC1(C)OB(c2ccnc3[nH]cc(F)c23)OC1(C)C ; OC1(c2ccc(-c3ccnc4[nH]cc(F)c34)cc2Cl)CCNCC1]

Step B: A microwave vial was charged with tert-butyl 4-(4-bromo-2-chlorophenyl)-4-hydroxypiperidine-1-carboxylate (0.070 g, 0.179 mmol), 3-fluoro-4-(4,4,5,5-tetramethyl-1,3,2-dioxaborolan-2-yl)-1H-pyrrolo[2,3-b]pyridine (0.052 g, 0.197 mmol), and tetrakis(triphenylphosphine)palladium(0) (10.35 mg, 8.96 μmol). The vial was sealed. Its contents were purged with nitrogen twice. Then, dioxane (0.560 mL) was added, followed by 2 M aqueous potassium carbonate (0.134 ml, 0.269 mmol). The content of the reaction vial was purged with nitrogen twice, and was then heated in the microwave reactor at 150° C. for 30 min. The reaction mixture was partitioned between 10 mL of water and 10 mL of ethyl acetate. The layers were separated; the aqueous layer was extracted with three 10 mL portions of ethyl acetate. The combined organic layer was washed with 10 mL of brine, was then dried over sodium sulfate, filtered, and was then concentrated under diminished pressure.

Step C: The crude material from step B was dissolved in dichloromethane (1.0 mL). It was then treated with trifluoroacetic acid (1.0 mL) for 30 min. The solvent and trifluoroacetic acid were then removed by rotary evaporation. The crude product was dissolved in methanol (0.5 mL). Purification by reverse-phase HPLC provided the title compound as the diformate salt. ¹H NMR (400 MHz, DMSO-d₆) δ 11.73 (s, 1H), 8.34 (d, J=4.9 Hz, 1H), 7.93 (d, J=8.1 Hz, 1H), 7.68 (dd, J=7.6, 2.7 Hz, 2H), 7.57 (s, 1H), 7.20 (d, J=4.9 Hz, 1H), 5.75 (s, 1H), 3.19 (d, J=10.9 Hz, 4H), 2.68 (td, J=13.2, 12.3, 5.7 Hz, 2H), 1.77 (d, J=13.6 Hz, 2H). LC-MS (m/z [M+H)]⁺): 346.1

### Compounds 98-109

Compounds 98-109 and 319 were prepared using an analogous procedure to that of Compound 97, and are shown in Table 18.

TABLE 18

| | | | | LCMS |
|---|---|---|---|---|
| Compound | Structure | IUPAC name | ¹H NMR | m/z |
| 98 | 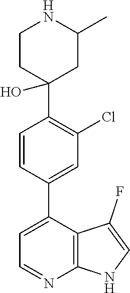 [SMILES: CC1CC(O)(c2ccc(-c3ccnc4[nH]cc(F)c34)cc2Cl)CCN1] | 4-(2-chloro-4-(3- fluoro-1H- pyrrolo[2,3- b]pyridin-4- yl)phenyl)-2- methylpiperidin- 4-ol | ¹H NMR (400 MHz, DMSO- d₆) δ 11.74 (s, 1H), 8.34 (d, J = 4.9 Hz, 1H), 8.29 (s, 1H), 7.76 (d, J = 8.3 Hz, 1H), 7.70 (dd, J = 2.8, 1.9 Hz, 1H), 7.64 (ddd, J = 8.2, 2.8, 1.9 Hz, 1H), 7.57 (d, J = 2.1 Hz, 1H), 7.20 (d, J = 4.9 Hz, 1H), 3.25 − 3.18 (m, 1H), 3.17 − 3.07 (m, 1H), 2.87 − 2.73 (m, 3H), 1.79 (t, J = 11.2 Hz, 1H), 1.63 (dd, J = 14.1, 8.7 Hz, 1H), 1.27 (d, J = 6.5 Hz, 3H). | [M + H]⁺ 360.1 |
| 99 | 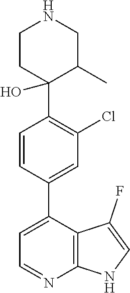 [SMILES: CC1CNCCC1(O)c1ccc(-c2ccnc3[nH]cc(F)c23)cc1Cl] | 4-(2-chloro-4-(3- fluoro-1H- pyrrolo[2,3- b]pyridin-4- yl)phenyl)-3- methylpiperidin- 4-ol | ¹H NMR (400 MHz, DMSO- d₆) δ 11.74 (s, 1H), 8.34 (d, J = 4.9 Hz, 1H), 7.98 (d, J = 8.0 Hz, 1H), 7.70 (dt, J = 4.1, 2.5 Hz, 2H), 7.57 (d, J = 7.2 Hz, 1H), 7.23 (d, J = 4.9 Hz, 1H), 5.70 (s, 1H), 3.25 − 2.83 (m, 6H), 1.71 (d, J = 13.8 Hz, 1H), 0.58 (d, J = 6.1 Hz, 3H). | [M + H]⁺ 360.1 |
| 100 | 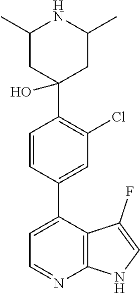 [SMILES: CC1CC(O)(c2ccc(-c3ccnc4[nH]cc(F)c34)cc2Cl)CC(C)N1] | 4-(2-chloro-4-(3- fluoro-1H- pyrrolo[2,3- b]pyridin-4- yl)phenyl)-2,6- dimethylpiperidin- 4-ol | | [M + H]⁺ 374.1 |
| 101 | 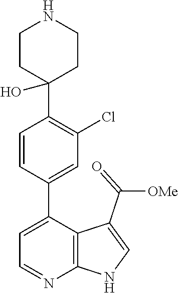 [SMILES: COC(=O)c1c[nH]c2nccc(-c3ccc(C4(O)CCNCC4)c(Cl)c3)c12] | methyl 4-(3- chloro-4-(4- hydroxypiperidin- 4-yl)phenyl)- 1H-pyrrolo[2,3- b]pyridine-3- carboxylate | ¹H NMR (400 MHz, DMSO- d₆) δ 8.37 (d, J = 4.9 Hz, 1H), 8.26 (d, J = 5.5 Hz, 1H), 7.86 (d, J = 8.6 Hz, 1H), 7.49 − 7.30 (m, 2H), 7.17 (d, J = 4.9 Hz, 1H), 5.67 (s, 1H), 3.26 (s, 3H), 3.35 − 3.17 (m, 4H), 2.72 (td, J = 15.1, 13.1, 7.1 Hz, 2H), 1.76 (d, J = 13.7 Hz, 2H). | M + H]⁺ 386.1 |
| 102 | 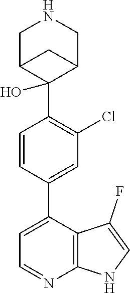 [SMILES: OC1(c2ccc(-c3ccnc4[nH]cc(F)c34)cc2Cl)C2CNCC1C2] | 6-(2-chloro-4-(3- fluoro-1H- pyrrolo[2,3- b]pyridin-4- yl)phenyl)-3- azabicyclo[3.1.1] heptan-6-ol | ¹H NMR (400 MHz, DMSO- d₆) δ 8.51 − 8.18 (m, 1H), 7.77 − 7.71 (m, 2H), 7.62 (dt, J = 8.2, 2.5 Hz, 1H), 7.56 (d, J = 2.0 Hz, 1H), 7.20 (d, J = 4.9 Hz, 1H), 3.67 (dd, J = 11.4, 3.2 Hz, 2H), 3.60 (d, J = 11.2 Hz, 2H), 2.91 (s, 2H), 1.44 − 1.01 (m, 2H). | [M + H]⁺ 358.1 |
| 103 | 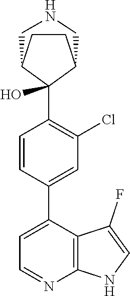 [SMILES: O[C@]1(c2ccc(-c3ccnc4[nH]cc(F)c34)cc2Cl)[C@@H]2CC[C@H]1CNC2] | (1R,5S,8s)-8-(2- chloro-4-(3- fluoro-1H- pyrrolo[2,3- b]pyridin-4- yl)phenyl)-3- azabicyclo[3.2.1] octan-8-ol | ¹H NMR (400 MHz, DMSO- d₆) δ 11.76 (s, 1H), 8.32 (d, J = 4.9 Hz, 1H), 7.72 − 7.66 (m, 1H), 7.66 − 7.53 (m, 3H), 7.20 (dd, J = 5.0, 2.3 Hz, 1H), 5.20 (d, J = 58.6 Hz, 1H), 3.71 (d, J = 11.7 Hz, 1H), 3.49 − 3.36 (m, 2H), 2.74 − 2.65 (m, 1H), 2.63 − 2.54 (m, 1H), 2.41 (dd, J = 11.8, 3.2 Hz, 1H), 1.73 − 1.33 (m, 4H). | [M + H]⁺ 372.1 |
| 104 | 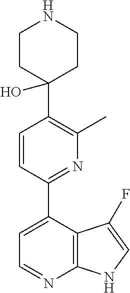 [SMILES: Cc1nc(-c2ccnc3[nH]cc(F)c23)ccc1C1(O)CCNCC1] | 4-(6-(3-fluoro- 1H-pyrrolo[2,3- b]pyridin-4-yl)- 2-methylpyridin- 3-yl)piperidin-4- ol | ¹H NMR (400 MHz, DMSO- d₆) δ 11.70 (s, 1H), 8.35 (d, J = 4.9 Hz, 1H), 7.92 (d, J = 8.3 Hz, 1H), 7.68 (dd, J = 8.2, 3.3 Hz, 1H), 7.60 − 7.51 (m, 1H), 7.43 (d, J = 4.9 Hz, 1H), 5.59 (s, 1H), 3.32 − 3.24 (m, 4H), 2.83 (s, 3H), 2.20 (dt, J = 16.9, 8.7 Hz, 2H), 2.04 (d, J = 13.7 Hz, 2H). | [M + H]⁺ 327.2 |
| 105 | 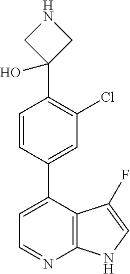 [SMILES: OC1(c2ccc(-c3ccnc4[nH]cc(F)c34)cc2Cl)CNC1] | 3-(2-chloro-4-(3- fluoro-1H- pyrrolo[2,3- b]pyridin-4- yl)phenyl)azetidin- 3-ol | ¹H NMR (400 MHz, DMSO- d₆) δ 11.75 (s, 1H), 8.35 (d, J = 4.9 Hz, 1H), 8.30 (s, 1H), 7.73 (dd, J = 2.7, 1.8 Hz, 1H), 7.69 − 7.61 (m, 1H), 7.61 − 7.55 (m, 2H), 7.21 (d, J = 4.9 Hz, 1H), 4.38 (d, J = 10.2 Hz, 2H), 3.94 (d, J = 10.2 Hz, 2H). | [M + H]⁺ 318.0 |
| 106 | 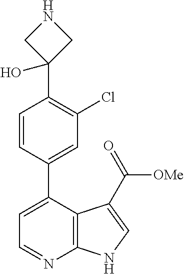 [SMILES: COC(=O)c1c[nH]c2nccc(-c3ccc(C4(O)CNC4)c(Cl)c3)c12] | methyl 4-(3- chloro-4-(3- hydroxyazetidin- 3-yl)phenyl)-1H- pyrrolo[2,3- b]pyridine-3- carboxylate | ¹H NMR (400 MHz, DMSO- d₆) δ 8.38 (d, J = 4.9 Hz, 1H), 8.32 (s, 1H), 8.26 (s, 1H), 7.48 (d, J = 7.9 Hz, 1H), 7.42 (d, J = 1.7 Hz, 1H), 7.36 (dd, J = 7.9, 1.8 Hz, 1H), 7.15 (d, J = 4.9 Hz, 1H), 4.37 (d, J = 10.3 Hz, 2H), 3.95 (d, J = 10.1 Hz, 2H), 3.32 (s, 3H). | [M + H]⁺ 358.1 |
| 107 | 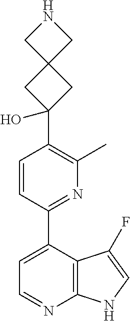 [SMILES: Cc1nc(-c2ccnc3[nH]cc(F)c23)ccc1C1(O)CC2(CNC2)C1] | 6-(6-(3-fluoro- 1H-pyrrolo[2,3- b]pyridin-4-yl)- 2-methylpyridin- 3-yl)-2- azaspiro[3.3] heptan-6-ol | ¹H NMR (400 MHz, DMSO- d₆) δ 11.68 (s, 1H), 8.39 (s, 1H), 8.35 (d, J = 4.9 Hz, 1H), 7.71 (d, J = 8.2 Hz, 1H), 7.66 − 7.59 (m, 1H), 7.55 (s, 1H), 7.42 (d, J = 4.9 Hz, 1H), 5.55 (s, 1H), 4.00 (s, 2H), 3.72 (s, 2H), 2.82 − 2.71 (m, 2H), 2.59 (s, 3H), 2.58 − 2.53 (m, 2H). | [M + H]+ 339.2 |
| 108 | 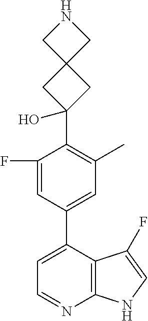 [SMILES: Cc1cc(-c2ccnc3[nH]cc(F)c23)cc(F)c1C1(O)CC2(CNC2)C1] | 6-(2-fluoro-4-(3- fluoro-1H- pyrrolo[2,3- b]pyridin-4-yl)- 6- methylphenyl)- 2- azaspiro[3.3] heptan-6-ol | ¹H NMR (400 MHz, DMSO- d₆) δ 8.31 (t, J = 4.7 Hz, 1H), 7.54 (dd, J = 5.8, 2.0 Hz, 1H), 7.37 − 7.29 (m, 1H), 7.27 − 7.17 (m, 2H), 7.14 (d, J = 4.9 Hz, 1H), 3.99 − 3.89 (m, 2H), 3.70 − 3.60 (m, 2H), 3.20 (d, J = 2.5 Hz, 1H), 2.70 (d, J = 11.6 Hz, 2H), 2.59 (d, J = 11.2 Hz, 2H), 2.46 (s, 1H), 2.40 (s, 3H). | [M + H]⁺ 356.2 |
| 109 | 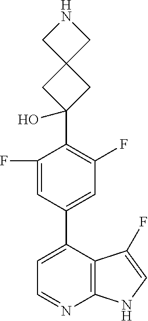 [SMILES: OC1(c2c(F)cc(-c3ccnc4[nH]cc(F)c34)cc2F)CC2(CNC2)C1] | 6-(2,6-difluoro- 4-(3-fluoro-1H- pyrrolo[2,3- b]pyridin-4- yl)phenyl)-2- azaspiro[3.3] heptan-6-ol | ¹H NMR (400 MHz, DMSO- d₆) δ 8.33 (d, J = 4.9 Hz, 1H), 7.58 (d, J = 1.9 Hz, 1H), 7.34 − 7.26 (m, 2H), 7.20 (d, J = 4.9 Hz, 1H), 3.93 (s, 2H), 3.66 (s, 2H), 2.73 (d, J = 11.8 Hz, 2H), 2.59 (d, J = 10.9 Hz, 2H), 2 | [M + H]⁺ 360.1 |
| 319 | 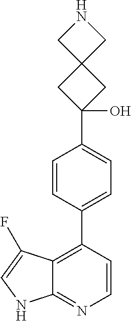 [SMILES: OC1(c2ccc(-c3ccnc4[nH]cc(F)c34)cc2)CC2(CNC2)C1] | 6-(4-(3-fluoro- 1H-pyrrolo[2,3- b]pyridin-4- yl)phenyl)-2- azaspiro[3.3] heptan-6-ol | ¹H NMR (400 MHz, Methanol- d₄) δ 8.26 (d, J = 5.0 Hz, 1H), 7.64 − 7.49 (m, 2H), 7.26 (d, J = 7.68 (dd, J = 8.4, 2.8 Hz, 2H), 2.1 Hz, 1H), 7.15 (d, J = 5.0 Hz, 1H), 4.20 (s, 2H), 3.98 (s, 2H), 2.93 − 2.84 (m, 2H), 2.66 − 2.52 (m, 2H). | [M + H]⁺ 324.2 |

### Compound 110

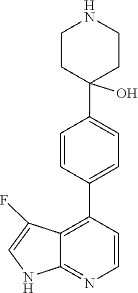 [SMILES: OC1(c2ccc(-c3ccnc4[nH]cc(F)c34)cc2)CCNCC1]

### 4-(4-(3-fluoro-1H-pyrrolo[2,3-b]pyridin-4-yl)phenyl)piperidin-4-ol

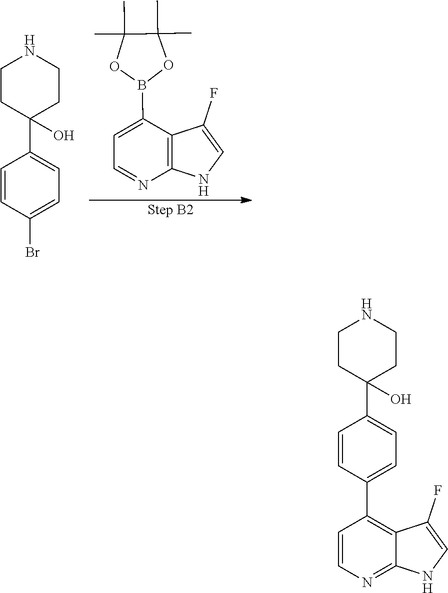 [SMILES: OC1(c2ccc(Br)cc2)CCNCC1 ; CC1(C)OB(c2ccnc3[nH]cc(F)c23)OC1(C)C ; OC1(c2ccc(-c3ccnc4[nH]cc(F)c34)cc2)CCNCC1]

Compounds of general formula A5 can also be synthesized as per step B2 below.

Step B2: A microwave vial was charged with 4-(4-bromophenyl)piperidin-4-ol (0.100 g, 0.390 mmol), 3-fluoro-4-(4,4,5,5-tetramethyl-1,3,2-dioxaborolan-2-yl)-1H-pyrrolo[2,3-b]pyridine (0.113 g, 0.429 mmol) and chloro(crotyl)(2-dicyclohexylphosphino-2′,4′,6′-triisopropyl-1,1′-biphenyl) palladium(II) (0.013 g, 0.020 mmol, XPhos Pd(crotyl)Cl). The content of the reaction vessel was then flushed with nitrogen. Then dioxane (1.170 ml) and 2 M aqueous potassium phosphate tribasic (0.293 ml, 0.586 mmol) were added. The content of the reaction vessel was then flushed with nitrogen. The resulting mixture was stirred at 80° C. for 2.5 hr. The reaction mixture was concentrated under diminished pressure. Purification by reverse phase HPLC gave the title product, as the diformic acid salt. H NMR (400 MHz, DMSO-d₆) δ 11.64 (s, 1H), 8.38 (s, 1H), 8.31 (d, J=4.9 Hz, 1H), 7.70-7.53 (m, 4H), 7.52 (s, 1H), 7.14 (d, J=4.9 Hz, 1H), 3.21-2.92 (, 4H), 2.05 (dd, J=13.1, 8.7 Hz, 2H), 1.72 (d, J=13.5 Hz, 2H). LC-MS (m/z [M+H])⁺): 312.1

### Compounds 111-116

Compounds 111-116 were synthesized in an analogous manner to that of Compound 110 and are shown in Table 19.

TABLE 19

| | | | | LCMS |
|---|---|---|---|---|
| Compound | Structure | IUPAC name | ¹H NMR | m/z |
| 111 | 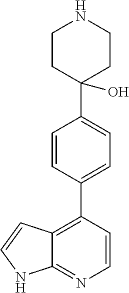 [SMILES: OC1(c2ccc(-c3ccnc4[nH]ccc34)cc2)CCNCC1] | 4-(4-(1H- pyrrolo[2,3- b]pyridin-4- yl)phenyl)piperidin- 4-ol | ¹H NMR (400 MHz, DMSO- d₆) δ 11.77 (s, 1H), 8.27 (d, J = 4.9 Hz, 1H), 7.76 − 7.71 (m, 2H), 7.67 − 7.61 (m, 2H), 7.53 (d, J = 3.5 Hz, 1H), 7.17 (d, J = 4.9 Hz, 1H), 6.62 (d, J = 3.5 Hz, 1H), 4.85 (s, 1H), 2.95 (td, J = 12.1, 2.5 Hz, 2H), 2.81 − 2.62 (m, 2H), 1.85 (td, J = 12.6, 4.5 Hz, 2H), 1.64 − 1.45 (m, 2H). | [M + H]⁺ 294.1 |
| 112 | 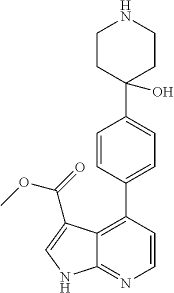 [SMILES: COC(=O)c1c[nH]c2nccc(-c3ccc(C4(O)CCNCC4)cc3)c12] | methyl 4-(4-(4- hydroxypiperidin- 4-yl)phenyl)-1H- pyrrolo[2,3- b]pyridine-3- carboxylate | ¹H NMR (400 MHz, DMSO- d₆) δ 12.60 (s, 1H), 8.55 (bs, 1H), 8.35 (d, J = 4.8 Hz, 1H), 8.21 (d, J = 3.0 Hz, 1H), 7.57 − 7.45 (m, 2H), 7.45 − 7.30 (m, 2H), 7.12 (d, J = 4.9 Hz, 1H), 5.52 (s, 1H), 3.30 − 3.21 (m, 4H), 3.16 (s, 3H), 2.17 (td, J = 15.8, 14.8, 5.8 Hz, 2H), 1.84 (d, J = 13.9 Hz, 2H). | [M + H]⁺ 352.2 |
| 113 | 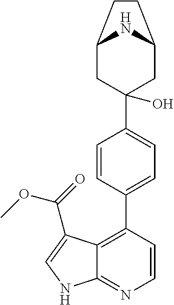 [SMILES: COC(=O)c1c[nH]c2nccc(-c3ccc(C4(O)C[C@H]5CC[C@@H](C4)N5)cc3)c12] | methyl 4-(4- ((1R,5S)-3- hydroxy-8- azabicyclo[3.2.1] octan-3-yl)phenyl)- 1H-pyrrolo[2,3- b]pyridine-3- carboxylate | ¹H NMR (400 MHz, DMSO- d₆) δ 8.43 (s, 1H), 8.34 (d, J = 4.8 Hz, 1H), 8.19 (s, 1H), 7.59 (d, J = 8.1 Hz, 2H), 7.32 (d, J = 8.1 Hz, 2H), 7.12 (d, J = 4.9 Hz, 1H), 5.75 (s, 1H), 3.92 (s, 2H), 3.13 (s, 3H), 2.40 (d, J = 7.4 Hz, 1H), 2.33 (d, J = 14.0 Hz, 2H), 1.91 (d, J = 14.4 Hz, 2H), 1.88 − 1.83 (m, 3H). | [M + H]⁺ 378.2 |
| 114 | 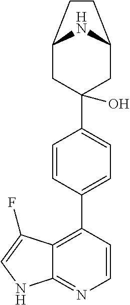 [SMILES: OC1(c2ccc(-c3ccnc4[nH]cc(F)c34)cc2)C[C@H]2CC[C@@H](C1)N2] | (1R,5S)-3-(4-(3- fluoro-1H- pyrrolo[2,3- b]pyridin-4- yl)phenyl)-8- azabicyclo[3.2.1] octan-3-ol | ¹H NMR (400 MHz, DMSO- d₆) δ 11.63 (s, 1H), 8.39 (s, 1H), 8.30 (d, J = 4.9 Hz, 1H), 7.68 − 7.58 (m, 4H), 7.51 (s, 1H), 7.14 (d, J = 4.9 Hz, 1H), 3.89 (s, 2H), 2.38 (d, J = 7.4 Hz, 1H), 2.35 − 2.25 (m, 2H), 1.91 (d, J = 14.5 Hz, 2H) 1.86 − 1.79 (m, 3H). | [M + H]⁺ 338.1 |
| 115 | 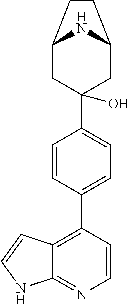 [SMILES: OC1(c2ccc(-c3ccnc4[nH]ccc34)cc2)C[C@H]2CC[C@@H](C1)N2] | (1R,5S)-3-(4-(1H- pyrrolo[2,3- b]pyridin-4- yl)phenyl)-8- azabicyclo[3.2.1] octan-3-ol | ¹H NMR (400 MHz, DMSO- d₆) δ 11.77 (s, 1H), 8.26 (d, J = 5.0 Hz, 1H), 7.75 − 7.69 (m, 2H), 7.66 (d, J = 8.6 Hz, 2H), 7.52 (dd, J = 3.6, 1.7 Hz, 1H), 7.17 (d, J = 5.0 Hz, 1H), 6.61 (dd, J = 3.4, 1.0 Hz, 1H), 4.91 (s, 1H), 3.58 (s, 2H), 2.36 − 2.22 (m, 2H), 2.15 (dd, J = 14.2, 3.7 Hz, 2H), 1.90 − 1.75 (m, 2H), 1.76 − 1.64 (m, 2H). | [M + H]⁺ 320.1 |
| 116 | 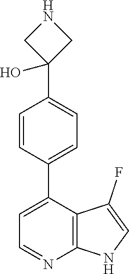 [SMILES: OC1(c2ccc(-c3ccnc4[nH]cc(F)c34)cc2)CNC1] | 3-(4-(3-fluoro-1H- pyrrolo[2,3- b]pyridin-4- yl)phenyl)azetidin- 3-ol | ¹H NMR (400 MHz, DMSO- d₆) δ 11.68 (s, 1H), 8.32 (d, J = 5.0 Hz, 2H), 7.77 − 7.64 (m, 4H), 7.53 (d, J = 2.0 Hz, 1H), 7.16 (dd, J = 4.7, 3.3 Hz, 1H), 4.34 (d, J = 10.9 Hz, 2H), 4.08 (d, J = 10.8 Hz, 2H). | [M + H]⁺ 284.1 |

### Compound 117

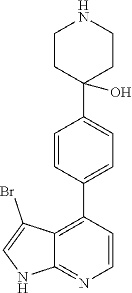 [SMILES: OC1(c2ccc(-c3ccnc4[nH]cc(Br)c34)cc2)CCNCC1]

### 4-(4-(3-bromo-1H-pyrrolo[2,3-b]pyridin-4-yl)phenyl)piperidin-4-ol

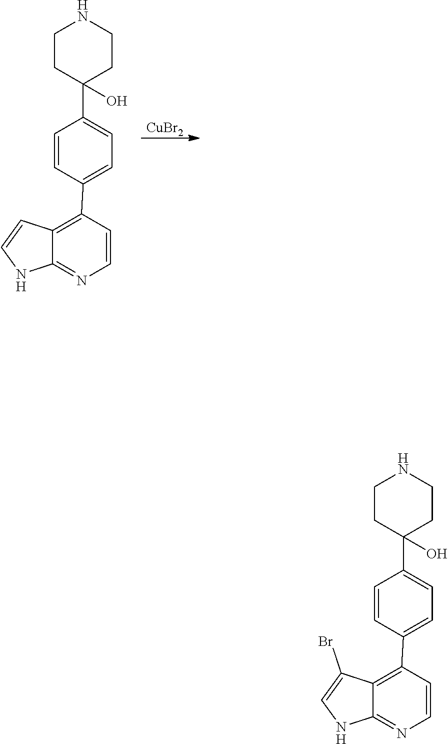

Step D: A vial was charged with 4-(4-(1H-pyrrolo[2,3-b]pyridin-4-yl)phenyl)piperidin-4-ol (0.175 g, 0.597 mmol), followed by copper (II) bromide (0.400 g, 1.79 mmol). Acetonitrile (5.0 ml) was added to the reaction and it was stirred for 0.1 h. Then ammonia in methanol (7 N, 10 mL) was added to the reaction mixture and it was stirred for a further 30 minutes. The reaction mixture was poured into aqueous sodium bicarbonate and extracted with ethyl acetate (2×). The organic phase was partitioned and the solvent removed by rotary evaporation to give the crude product. Further purification by reverse phase HPLC gave the title product as the di-trifluoroacetate salt. ¹H NMR (400 MHz, DMSO-d₆) δ 12.26 (s, 1H), 8.53 (bs, 1H), 8.31 (d, J=4.8 Hz, 1H), 7.73 (d, J=2.7 Hz, 1H), 7.63-7.43 (m, 4H), 7.02 (d, J=4.8 Hz, 1H), 5.54 (s, 1H), 3.30-3.21 (m, 4H), 2.30-2.02 (m, 2H), 1.85 (d, J=13.9 Hz, 2H). LC-MS (m/z [M+H)]⁺): 372.1

### Compounds 118-125

Compounds 118-125 were synthesized in an analogous manner to that of Compound 117, and are shown in Table 20.

TABLE 20

| | | | | LCMS |
|---|---|---|---|---|
| Compound | Structure | IUPAC name | ¹H NMR | m/z |
| 118 | 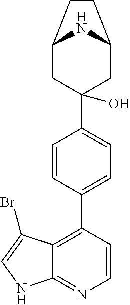 [SMILES: OC1(c2ccc(-c3ccnc4[nH]cc(Br)c34)cc2)C[C@H]2CC[C@@H](C1)N2] | (1R,5S)-3-(4-(3- bromo-1H- pyrrolo[2,3- b]pyridin-4- yl)phenyl)-8- azabicyclo[3.2.1] octan-3-ol | ¹H NMR (400 MHz, DMSO- d₆) δ 12.27 (s, 1H), 8.45 (s, 1H), 8.29 (d, J = 4.8 Hz, 1H), 7.71 (s, 1H), 7.66 − 7.59 (m, 2H), 7.50 − 7.43 (m, 2H), 7.01 (d, J = 4.9 Hz, 1H), 3.91 (s, 2H), 2.44 − 2.27 (m, 4H), 1.95 − 1.83 (m, 4H). | [M + H]⁺ 398.1 |
| 119 | 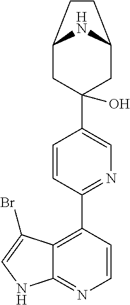 [SMILES: OC1(c2ccc(-c3ccnc4[nH]cc(Br)c34)nc2)C[C@H]2CC[C@@H](C1)N2] | (1R,5S)-3-(6-(3- bromo-1H- pyrrolo[2,3- b]pyridin-4- yl)pyridin-3-yl)-8- azabicyclo[3.2.1] octan-3-ol | ¹H NMR (400 MHz, DMSO- d₆) δ 12.30 (s, 1H), 8.86 (d, J = 2.3 Hz, 1H), 8.70 (d, J = 9.8 Hz, 2H), 8.36 (d, J = 4.9 Hz, 1H), 8.01 (dd, J = 8.1, 2.4 Hz, 1H), 7.76 (d, J = 2.7 Hz, 1H), 7.65 (d, J = 8.2 Hz, 1H), 7.17 (d, J = 4.8 Hz, 1H), 5.75 (s, 1H), 5.62 (s, 1H), 4.12 (s, 3H), 2.54 (s, 1H), 2.48 − 2.27 (m, 3H), 2.06 (d, J = 14.9 Hz, 2H), 2.02 − 1.88 (m, 2H). | [M + H]⁺ 399.0 |
| 120 | 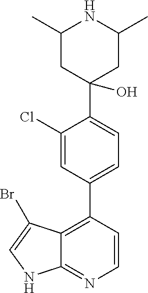 [SMILES: CC1CC(O)(c2ccc(-c3ccnc4[nH]cc(Br)c34)cc2Cl)CC(C)N1] | 4-(4-(3-bromo- 1H-pyrrolo[2,3- b]pyridin-4-yl)-2- chlorophenyl)-2,6- dimethylpiperidin- 4-ol | | [M + H]⁺ 436.1 |
| 121 | 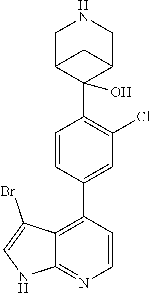 [SMILES: OC1(c2ccc(-c3ccnc4[nH]cc(Br)c34)cc2Cl)C2CNCC1C2] | 6-(4-(3-bromo- 1H-pyrrolo[2,3- b]pyridin-4-yl)-2- chlorophenyl)-3- azabicyclo[3.1.1] heptan-6-ol | | [M + H]⁺ 420.0 |
| 122 | 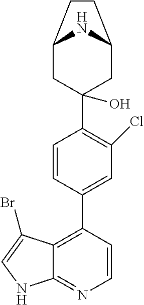 [SMILES: OC1(c2ccc(-c3ccnc4[nH]cc(Br)c34)cc2Cl)C[C@H]2CC[C@@H](C1)N2] | (1R,5S)-3-(4-(3- bromo-1H- pyrrolo[2,3- b]pyridin-4-yl)-2- chlorophenyl)-8- azabicyclo[3.2.1] octan-3-ol | ¹H NMR (400 MHz, DMSO- d₆) δ 12.33 (s, 1H), 8.93 (d, J = 11.0 Hz, 1H), 8.33 (d, J = 4.8 Hz, 1H), 7.87 (d, J = 8.1 Hz, 1H), 7.77 (d, J = 2.8 Hz, 2H), 7.53 (d, J = 7.5 Hz, 2H), 7.06 (d, J = 4.8 Hz, 1H), 5.75 (s, 1H), 5.68 (s, 1H), 4.15 (s, 2H), 2.86 (dd, J = 15.3, 3.7 Hz, 2H), 2.46 (d, J = 8.0 Hz, 2H), 2.03 − 1.92 (m, 4H). | [M + H]⁺ 434.0 |
| 123 | 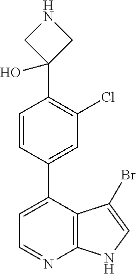 [SMILES: OC1(c2ccc(-c3ccnc4[nH]cc(Br)c34)cc2Cl)CNC1] | 3-(4-(3-bromo- 1H-pyrrolo[2,3- b]pyridin-4-yl)-2- chlorophenyl) azetidin-3-ol | ¹H NMR (400 MHz, DMSO- d₆) δ 8.36 − 8.30 (m, 2H), 7.78 (s, 1H), 7.58 (d, J = 1.6 Hz, 1H), 7.57 − 7.46 (m, 2H), 7.06 (d, J = 4.8 Hz, 1H), 4.41 (d, J = 10.4 Hz, 2H), 3.97 (d, J = 10.4 Hz, 2H), 2.54 (s, 1H). | [M + H]⁺ 380.0 |
| 124 | 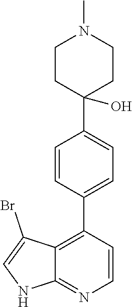 [SMILES: CN1CCC(O)(c2ccc(-c3ccnc4[nH]cc(Br)c34)cc2)CC1] | 4-(4-(3-bromo- 1H-pyrrolo[2,3- b]pyridin-4- yl)phenyl)-1- methylpiperidin-4- ol | ¹H NMR (400 MHz, DMSO- d₆) δ 12.27 (s, 1H), 9.34 (s, 1H), 8.31 (d, J = 4.9 Hz, 1H), 7.74 (d, J = 2.7 Hz, 1H), 7.66 − 7.45 (m, 4H), 7.02 (d, J = 4.8 Hz, 1H), 5.59 (s, 1H), 3.47 − 3.26 (m, 4H), 2.88 (d, J = 4.5 Hz, 3H), 2.27 − 2.12 (m, 2H), 1.92 (d, J = 14.0 Hz, 2H). | [M + H]⁺ 388.1 |
| 125 | 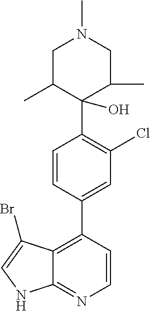 [SMILES: CC1CN(C)CC(C)C1(O)c1ccc(-c2ccnc3[nH]cc(Br)c23)cc1Cl] | 4-(4-(3-bromo- 1H-pyrrolo[2,3- b]pyridin-4-yl)-2- chlorophenyl)- 1,3,5- trimethylpiperidin- 4-ol | ¹H NMR (400 MHz, DMSO- d₆) δ 12.31 (s, 1H), 8.32 (d, J = 4.8 Hz, 1H), 8.15 (s, 1H), 7.90 (d, J = 8.1 Hz, 1H), 7.75 (d, J = 2.7 Hz, 1H), 7.48 (d, J = 1.8 Hz, 1H), 7.48 − 7.44 (m, 1H), 7.10 (d, J = 4.8 Hz, 1H), 4.84 (s, 1H), 3.12 − 3.02 (m, 2H), 2.77 − 2.66 (m, 2H), 2.44 − 2.33 (m, 4H), 0.56 (d, J = 6.9 Hz, 6H). | [M + H]⁺ 450.1 |

### Compound 126

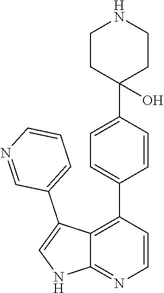 [SMILES: OC1(c2ccc(-c3ccnc4[nH]cc(-c5cccnc5)c34)cc2)CCNCC1]

### 4-(4-(3-(pyridin-3-yl)-1H-pyrrolo[2,3-b]pyridin-4-yl)phenyl)piperidin-4-ol

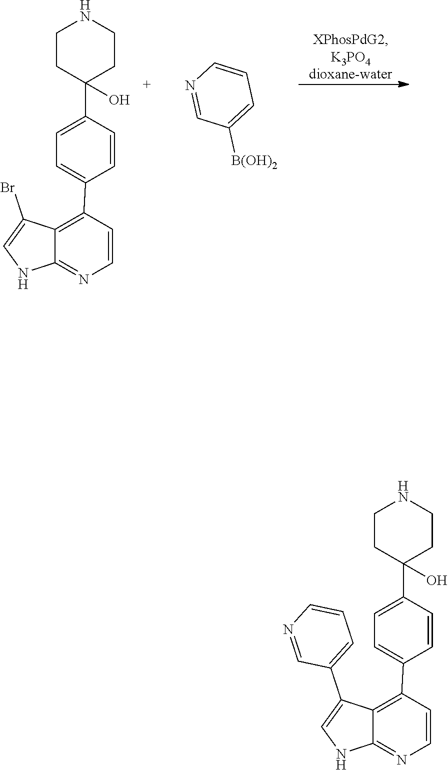 [SMILES: OC1(c2ccc(-c3ccnc4[nH]cc(Br)c34)cc2)CCNCC1 ; OB(O)c1cccnc1 ; "XPhosPdG2,K3PO4dioxane-water" ; OC1(c2ccc(-c3ccnc4[nH]cc(-c5cccnc5)c34)cc2)CCNCC1]

Step E: A microwave vial was charged with 4-(4-(3-bromo-1H-pyrrolo[2,3-b]pyridin-4-yl)phenyl)piperidin-4-ol (0.075 g, 0.353 mmol), pyridin-3-ylboronic acid (0.040 g, 0.325 mmol), potassium phosphate tribasic (0.075 g, 0.353 mmol), and Chloro(2-dicyclohexylphosphino-2′,4′,6′-triisopropyl-1,1′-biphenyl)[2-(2′-amino-1,1′-biphenyl)]palladium(II) (13 mg, 17 μmol, xPhos Pd G2). The vial was capped, and its content was purged with nitrogen twice. Then, 2 mL of 4:1 dioxane-water were added. The content of the reaction vessel was purged with nitrogen, then the reaction mixture was heated in the microwave reactor at 110° C. for 45 min. The reaction mixture was partitioned between 5 mL of water and 5 mL of dichloromethane. The layers were separated; the aqueous layer was extracted with three 5-mL portions of dichloromethane. The combined organic layer was washed with 5 mL of brine, was then dried (sodium sulfate), and was then concentrated under diminished pressure. The residue was purified by reverse-phase HPLC to provide the title compound; ¹H NMR (400 MHz, DMSO-d₆) δ 12.27 (s, 0.6×1H), 11.83 (s, 0.4×1H), 8.49 (s, 1H), 8.37 (d, J=4.9 Hz, 0.6×1H), 8.29 (d, J=4.9 Hz, 1H+0.6×1H), 8.23 (s, 1H), 7.81 (d, J=7.7 Hz, 1H=0.6×1H), 7.63 (d, J=8.1 Hz, 1H), 7.55 (s, 0.4×1H), 7.24-7.17 (m, 2H+0.4×1H), 7.17-7.10 (m, 2H), 7.06 (s, 0.6×1H), 6.63 (s, 0.4×1H), 3.32-3.20 (m, 4H), 2.23-2.11 (m, 0.4×1H), 2.04 (td, J=14.2, 13.8, 5.8 Hz, 1H+0.4×1H), 1.85 (d, J=13.8 Hz, 1H+0.4×1H). LC-MS (m/z [M+H)]⁺): 371.2

### Compounds 127-128

Compounds 127-128 were synthesized in an analogous manner to that of Compound 126, and are shown in Table 21.

TABLE 21

| Com- | | | ¹H | LCMS |
|---|---|---|---|---|
| pound | Structure | IUPAC name | NMR | m/z |
| 127 | 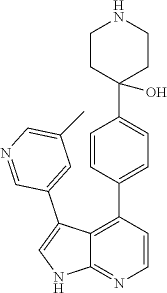 [SMILES: Cc1cncc(-c2c[nH]c3nccc(-c4ccc(C5(O)CCNCC5)cc4)c23)c1] | 4-(4-(3-(5- methylpyridin-3-yl)- 1H-pyrrolo[2,3- b]pyridin-4- yl)phenyl)piperidin- 4-ol | | [M + H]⁺ 385.2 |
| 128 | 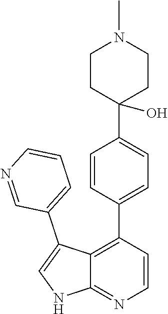 [SMILES: CN1CCC(O)(c2ccc(-c3ccnc4[nH]cc(-c5cccnc5)c34)cc2)CC1] | 1-methyl-4-(4-(3- (pyridin-3-yl)-1H- pyrrolo[2,3- b]pyridin-4- yl)phenyl)piperidin- 4-ol | | [M + H]⁺ 385.3 |

### Compound 129

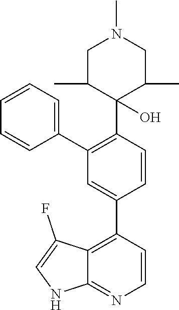 [SMILES: CC1CN(C)CC(C)C1(O)c1ccc(-c2ccnc3[nH]cc(F)c23)cc1-c1ccccc1]

### 4-(5-(3-fluoro-1H-pyrrolo[2,3-b]pyridin-4-yl)-[1,1′-biphenyl]-2-yl)-1,3,5-trimethylpiperidin-4-ol

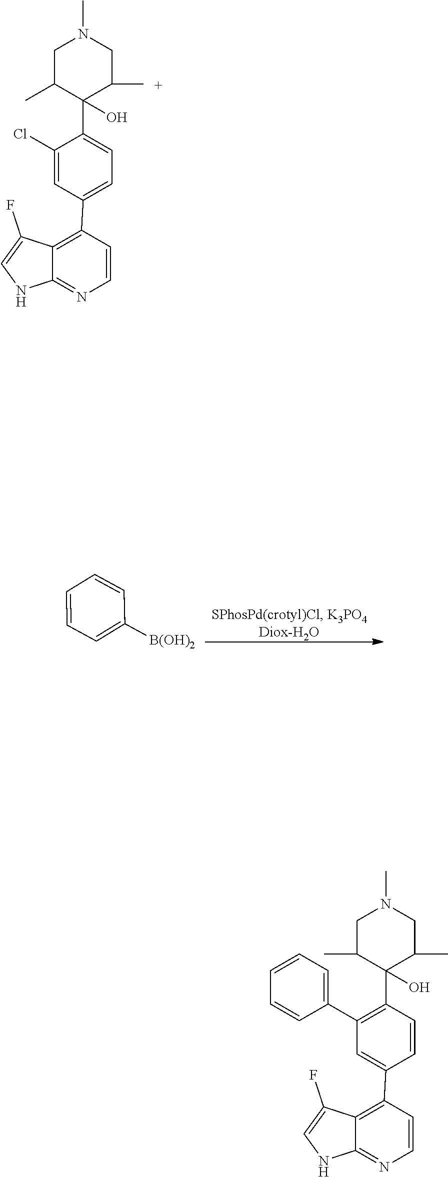

Step H: A microwave vial was charged with 4-(2-chloro-4-(3-fluoro-1H-pyrrolo[2,3-b]pyridin-4-yl)phenyl)-1,3,5-trimethylpiperidin-4-ol (0.030 g, 0.077 mmol), phenylboronic acid (0.014 g, 0.116 mmol) and chloro(crotyl)(2-dicyclohexylphosphino-2′,6′-dimethoxy-1,1′-biphenyl)palladium(II) (4.7 mg, 7.73 μmol, SphosPd(crotyl)Cl). The vial was capped, and its content was purged with nitrogen twice. Then, dioxane (0.465 mL) was added, followed by 2 M aqueous potassium phosphate tribasic (0.120 mL, 0.240 mmol). The content of the reaction vessel was purged with nitrogen, then the reaction mixture was heated in the microwave reactor at 150° C. for 30 min. The reaction mixture was partitioned between 5 mL of water and 5 mL of dichloromethane. The layers were separated; the aqueous layer was extracted with three 5-mL portions of dichloromethane. The combined organic layer was washed with 5 mL of brine, was then dried (sodium sulfate), and was then concentrated under diminished pressure. The residue was purified by reverse-phase HPLC to provide the title compound; ¹H NMR (400 MHz, DMSO-d₆) δ 11.62 (s, 1H), 8.28 (d, J=4.9 Hz, 1H), 8.16 (s, 1H), 7.98 (d, J=8.4 Hz, 1H), 7.68 (d, J=8.6 Hz, 1H), 7.49 (d, J=2.1 Hz, 1H), 7.41 (d, J=7.0 Hz, 2H), 7.28-7.23 (m, 2H), 7.19 (d, J=4.7 Hz, 2H), 4.49 (s, 1H), 2.35-2.21 (m, 2H), 2.22-2.11 (m, 1H), 2.07 (s, 3H), 2.04-1.90 (m, 1H), 0.63 (d, J=6.7 Hz, 1H), 0.57 (d, J=6.8 Hz, 6H), 0.51 (d, J=6.3 Hz, 1H). LC-MS (m/z [M+H)]⁺): 430.2

### Compound 130

Compound 130 was synthesized in an analogous manner to that of Compound 129, and is shown in Table 22.

TABLE 22

| | | | | LCMS |
|---|---|---|---|---|
| Compound | Structure | IUPAC name | ¹H NMR | m/z |
| 130 | 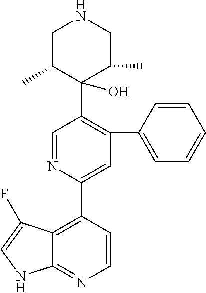 [SMILES: C[C@@H]1CNC[C@H](C)C1(O)c1cnc(-c2ccnc3[nH]cc(F)c23)cc1-c1ccccc1] | (3S,5R)-4-(6-(3- fluoro-1H- pyrrolo[2,3- b]pyridin-4-yl)-4- phenylpyridin-3- yl)-3,5- dimethylpiperidin- 4-ol | ¹H NMR (400 MHz, DMSO-d₆) δ 11.69 (s, 1H), 9.13 (s, 1H), 8.36 (d, J = 4.9 Hz, 1H), 7.56 (d, J = 4.9 Hz, 1H), 7.53 (d, J = 1.9 Hz, 1H), 7.48 − 7.44 (m, 2H), 7.39 − 7.32 (m, 1H), 7.30 − 7.20 (m, 2H), 4.73 (s, 1H), 2.62 (q, J = 13.8, 12.9 Hz, 2H), 2.41 (dd, J = 12.0, 4.1 Hz, 2H), 1.77 (dt, J = 11.6, 5.5 Hz, 2H), 0.63 − 0.51 (m, 6H). | [M + H]⁺ 417.2 |

### Compound 131

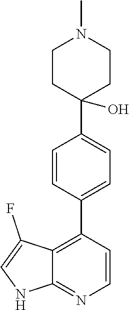 [SMILES: CN1CCC(O)(c2ccc(-c3ccnc4[nH]cc(F)c34)cc2)CC1]

### 4-(4-(3-fluoro-1H-pyrrolo[2,3-b]pyridin-4-yl)phenyl)-1-methylpiperidin-4-ol

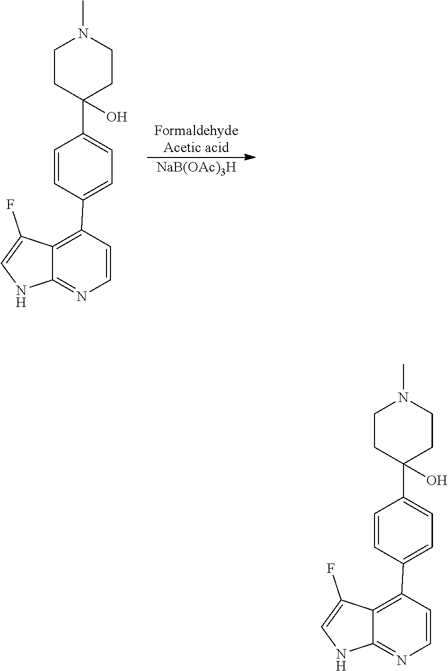

Step G: A solution containing 4-(4-(3-fluoro-1H-pyrrolo[2,3-b]pyridin-4-yl)phenyl)piperidin-4-ol (10 mg, 0.032 mmol) in 1:1 methanol (0.161 ml)-tetrahydrofuran (0.161 ml) was treated with formaldehyde (37% aqueous) (20 μl, 0.269 mmol), sodium triacetoxyborohydride (10 mg, 0.047 mmol) and acetic acid (10 μl, 0.175 mmol) at 23° C. for 20 hr. The reaction mixture was quenched by being poured into aqueous sodium bicarbonate and extracted into ethyl acetate (2×). The organic layers were partitioned, combined, and the and the solvent was removed by rotary evaporation. The crude product was purified by reverse phase PLC to give the title product as the bis-trifluoroacetate salt. ¹H NMR (400 MHz, DMSO-d₆) δ 11.66 (s, 1H), 9.33 (s, 1H), 8.32 (d, J=4.9 Hz, 1H), 7.68 (dd, J=8.4, 2.8 Hz, 2H), 7.60 (d, J=8.3 Hz, 2H), 7.53 (t, J=2.5 Hz, 1H), 7.15 (d, J=4.9 Hz, 1H), 5.59 (s, 1H), 3.43-3.28 (i, 4H), 2.88 (d, J=4.6 Hz, 3H), 2.23-52.08 (m, 2H), 1.98-1.83 (0, 2H). LC-MS (m/z [M+H)]⁺): 326.2

### Compounds 132-140

Compounds 132-140 were synthesized in an analogous manner to that of Compound 131, and are shown in Table 23.

TABLE 23

| | | | | LCMS |
|---|---|---|---|---|
| Compound | Structure | IUPAC name | ¹H NMR | m/z |
| 132 | 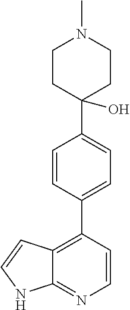 [SMILES: CN1CCC(O)(c2ccc(-c3ccnc4[nH]ccc34)cc2)CC1] | 4-(4-(1H- pyrrolo[2,3- b]pyridin-4- yl)phenyl)-1- methylpiperidin- 4-ol | ¹H NMR (400 MHz, DMSO- d₆) δ 11.85 (s, 1H), 9.37 (s, 1H), 8.30 (d, J = 5.0 Hz, 1H), 7.81 (d, J = 8.1 Hz, 2H), 7.64 (d, J = 8.1 Hz, 2H), 7.56 (t, J = 3.0 Hz, 1H), 7.21 (d, J = 5.0 Hz, 1H), 6.63 (d, J = 3.0 Hz, 1H), 5.60 (s, 1H), 3.36 (dt, J = 34.4, 11.6 Hz, 4H), 2.89 (d, J = 4.5 Hz, 3H), 2.28 − 2.11 (m, 1H), 1.92 (d, J = 14.1 Hz, 2H). | [M + H]⁺ 308.3 |
| 133 | 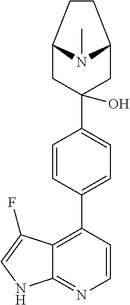 | (1R,5S)-3-(4-(3- fluoro-1H- pyrrolo[2,3- b]pyridin-4- yl)phenyl)-8- methyl-8- azabicyclo[3.2.1] octan-3-ol | ¹H NMR (400 MHz, DMSO- d₆) δ 11.63 (s, 1H), 8.39 − 8.07 (m, 2H), 7.61 (d, J = 1.3 Hz, 4H), 7.51 (dd, J = 2.8, 2.0 Hz, 1H), 7.13 (d, J = 4.9 Hz, 1H), 2.46 − 2.42 (m, 4H), 2.33 − 2.24 (m, 5H), 2.00 (s, 2H), 1.85 (d, J = 14.1 Hz, 2H). | [M + H]⁺ 450.1 |
| 134 | 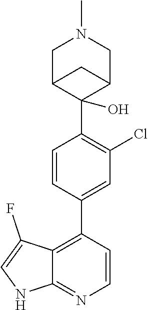 [SMILES: CN1CC2CC(C1)C2(O)c1ccc(-c2ccnc3[nH]cc(F)c23)cc1Cl] | 6-(2-chloro-4-(3- fluoro-1H- pyrrolo[2,3- b]pyridin-4- yl)phenyl)-3- methyl-3- azabicyclo[3.1.1] heptan-6-ol | ¹H NMR (400 MHz, DMSO- d₆) δ 11.72 (s, 1H), 8.34 (d, J = 4.9 Hz, 1H), 8.18 (s, 1H), 7.73 (dd, J = 5.1, 2.9 Hz, 2H), 7.68 − 7.60 (m, 1H), 7.57 (d, J = 2.5 Hz, 1H), 7.22 (d, J = 4.9 Hz, 1H), 3.22 (d, J = 10.0 Hz, 2H), 3.08 (d, J = 9.9 Hz, 2H), 2.96 (s, 2H), 2.40 (s, 3H), 1.60 (d, J = 9.6 Hz, 1H), 1.34 (dd, J = 9.7, 5.6 Hz, 1H). | [M + H]⁺ 372.2 |
| 135 | 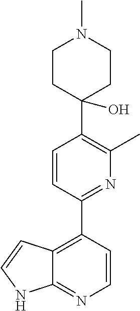 [SMILES: Cc1nc(-c2ccnc3[nH]ccc23)ccc1C1(O)CCN(C)CC1] | 1-methyl-4-(2- methyl-6-(1H- pyrrolo[2,3- b]pyridin-4- yl)pyridin-3- yl)piperidin-4-ol | ¹H NMR (400 MHz, DMSO- d₆) δ 11.80 (s, 1H), 8.32 (d, J = 5.0 Hz, 1H), 7.92 (s, 2H), 7.60 (d, J = 5.0 Hz, 1H), 7.57 (dd, J = 3.6, 2.5 Hz, 1H), 7.04 (dd, J = 3.5, 2.0 Hz, 1H), 3.44 − 3.29 (m, 4H), 2.88 (s, 3H), 2.87 (s, 3H), 2.34 − 2.18 (m, 2H), 2.11 (d, J = 13.7 Hz, 2H). | [M + H]⁺ 323.3 |
| 136 | 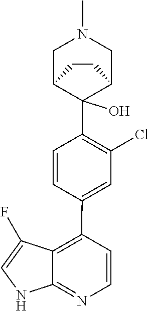 [SMILES: CN1C[C@H]2CC[C@@H](C1)C2(O)c1ccc(-c2ccnc3[nH]cc(F)c23)cc1Cl] | (1R,5S,8s)-8-(2- chloro-4-(3- fluoro-1H- pyrrolo[2,3- b]pyridin-4- yl)phenyl)-3- methyl-3- azabicyclo[3.2.1] octan-8-ol | ¹H NMR (400 MHz, DMSO- d₆) δ 11.71 (s, 1H), 8.33 (d, J = 4.9 Hz, 1H), 8.16 (d, J = 1.4 Hz, 1H), 7.69 (d, J = 2.8 Hz, 1H), 7.63 − 7.53 (m, 3H), 7.20 (d, J = 4.9 Hz, 1H), 5.21 (s, 1H), 3.17 (s, 1H), 2.92 (d, J = 10.0 Hz, 2H), 2.80 − 2.63 (m, 2H), 2.54 (d, J = 7.3 Hz, 1H), 2.28 (s, 3H), 1.78 − 1.67 (m, 2H). | [M + H]⁺ 386.1 |
| 137 | 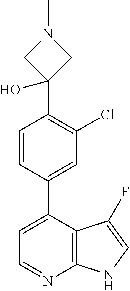 [SMILES: CN1CC(O)(c2ccc(-c3ccnc4[nH]cc(F)c34)cc2Cl)C1] | 3-(2-chloro-4-(3- fluoro-1H- pyrrolo[2,3- b]pyridin-4- yl)phenyl)-1- methylazetidin- 3-ol | ¹H NMR (400 MHz, DMSO- d₆) δ 11.73 (s, 1H), 8.34 (d, J = 4.9 Hz, 1H), 8.15 (s, 1H), 7.69 (t, J = 2.1 Hz, 1H), 7.66 − 7.49 (m, 3H), 7.19 (d, J = 4.9 Hz, 1H), 6.10 (s, 1H), 3.88 (d, J = 8.5 Hz, 2H), 3.67 (d, J = 8.4 Hz, 2H), 2.42 (s, 3H). | [M + H]⁺ 332.1 |
| 138 | 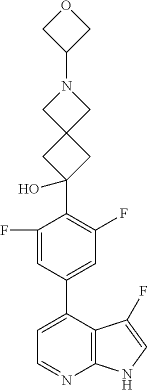 [SMILES: OC1(c2c(F)cc(-c3ccnc4[nH]cc(F)c34)cc2F)CC2(CN(C3COC3)C2)C1] | 6-(2,6-difluoro- 4-(3-fluoro-1H- pyrrolo[2,3- b]pyridin-4- yl)phenyl)-2- (oxetan-3-yl)-2- azaspiro[3.3] heptan-6-ol | ¹H NMR (400 MHz, DMSO- d₆) δ 11.76 (s, 1H), 8.33 (d, J = 4.9 Hz, 1H), 7.59 (s, 1H), 7.31 (d, J = 9.1 Hz, 2H), 7.20 (dd, J = 4.8, 1.4 Hz, 1H), 5.75 (d, J = 3.2 Hz, 1H), 4.52 (t, J = 6.5 Hz, 2H), 4.32 (t, J = 5.9 Hz, 2H), 3.65 (q, J = 6.1 Hz, 1H), 3.38 (s, 2H), 3.12 (s, 2H), 2.71 (d, J = 12.0 Hz, 2H), 2.53 (d, J = 18.0 Hz, 2H). | [M + H]⁺ 416.2 |
| 139 | 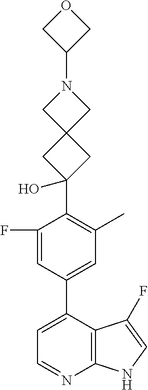 [SMILES: Cc1cc(-c2ccnc3[nH]cc(F)c23)cc(F)c1C1(O)CC2(CN(C3COC3)C2)C1] | 6-(2-fluoro-4-(3- fluoro-1H- pyrrolo[2,3- b]pyridin-4-yl)- 6-methylphenyl)- 2-(oxetan-3-yl)- 2- azaspiro[3.3] heptan-6-ol | ¹H NMR (400 MHz, DMSO- d₆) δ 11.68 (s, 1H), 8.31 (d, J = 4.9 Hz, 1H), 7.54 (s, 1H), 7.25 (s, 1H), 7.20 (d, J = 12.3 Hz, 1H), 7.15 (d, J = 4.9 Hz, 1H), 5.49 (s, 1H), 4.53 (t, J = 6.5 Hz, 2H), 4.32 (t, J = 5.8 Hz, 2H), 3.66 (q, J = 6.0 Hz, 1H), 3.40 (s, 2H), 3.12 (s, 2H), 2.69 (d, J = 12.4 Hz, 2H), 2.55 (d, J = 10.7 Hz, 2H), 2.40 (s, 3H). | [M + H]⁺ 412.2 |
| 140 | 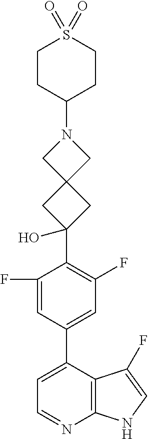 [SMILES: O=S1(=O)CCC(N2CC3(C2)CC(O)(c2c(F)cc(-c4ccnc5[nH]cc(F)c45)cc2F)C3)CC1] | 4-(6-(2,6- difluoro-4-(3- fluoro-1H- pyrrolo[2,3- b]pyridin-4- yl)phenyl)-6- hydroxy-2- azaspiro[3.3] heptan-2- yl)tetrahydro- 2H-thiopyran 1,1-dioxide | ¹H NMR (400 MHz, DMSO- d₆) δ 11.76 (s, 1H), 8.33 (d, J = 4.9 Hz, 1H), 7.59 (s, 1H), 7.31 (d, J = 8.8 Hz, 2H), 7.20 (d, J = 5.2 Hz, 1H), 5.76 (d, J = 2.0 Hz, 1H), 3.38 (s, 2H), 3.14 − 3.01 (m, 4H), 2.97 − 2.89 (m, 2H), 2.71 (d, J = 11.9 Hz, 2H), 2.53 (d, J = 9.3 Hz, 2H), 1.90 (t, J = 11.9 Hz, 2H), 1.80 − 1.70 (m, 2H). | [M + H]⁺ 492.2 |

### Compound 141

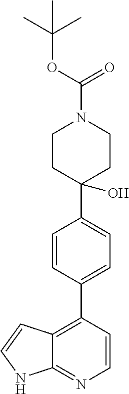 [SMILES: CC(C)(C)OC(=O)N1CCC(O)(c2ccc(-c3ccnc4[nH]ccc34)cc2)CC1]

### tert-butyl 4-(4-(1H-pyrrolo[2,3-b]pyridin-4-yl)phenyl)-4-hydroxypiperidine-1-carboxylate

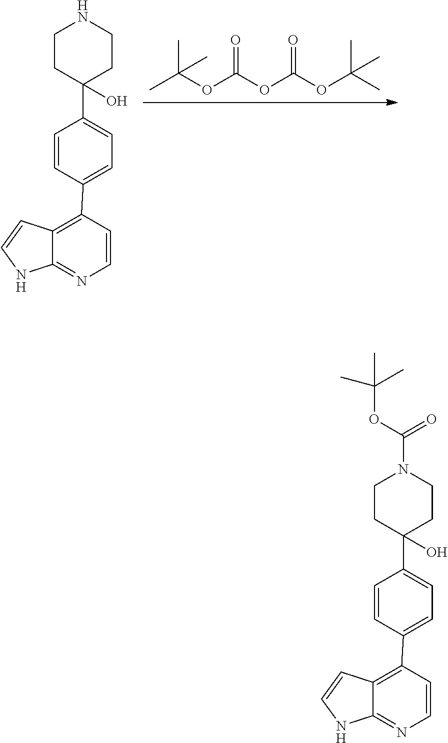

A suspension containing 4-(4-(1H-pyrrolo[2,3-b]pyridin-4-yl)phenyl)piperidin-4-ol (0.200 g, 0.682 mmol) in THE (2.0 mL) was treated with 1 M aqueous sodium hydroxide (1.023 mL, 1.023 mmol) and with di-tert-butyl dicarbonate (0.190 mL, 0.818 mmol) at 23° C. for 2 hr. The reaction mixture was partitioned between 10 mL of water and 10 mL of tert-butylmethyl ether. The layers were separated; the aqueous layer was extracted with three 10 mL portions of tert-butylmethyl ether. The combined organic layer was washed with 10 mL of brine, was then dried over sodium sulfate, filtered, and was then concentrated under diminished pressure. Purification by silica gel chromotography gave the title product. ¹H NMR (400 MHz, DMSO-d₆) δ 11.76 (s, 1H), 8.26 (d, J=4.9 Hz, 1H), 7.76-7.68 (m, 2H), 7.67-7.61 (m, 2H), 7.53 (dd, J=3.5, 2.5 Hz, 1H), 7.17 (d, J=5.0 Hz, 1H), 6.61 (dd, J=3.5, 1.9 Hz, 1H), 5.18 (s, 1H), 3.88 (d, J=12.5 Hz, 2H), 3.26-3.07 (m, 2H), 1.86 (td, J=13.1, 4.8 Hz, 2H), 1.72-1.56 (m, 2H), 1.43 (s, 9H). LC-MS (m/z [M+H)]⁺). 394.2

### Compound 142

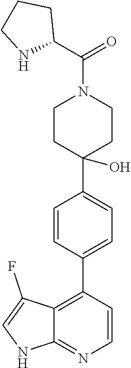 [SMILES: O=C([C@H]1CCCN1)N1CCC(O)(c2ccc(-c3ccnc4[nH]cc(F)c34)cc2)CC1]

### (R)-3-fluoro-4-(4-(4-hydroxy-1-propylpiperidin-4-yl)phenyl)-1H-pyrrolo[2,3-b]pyridine

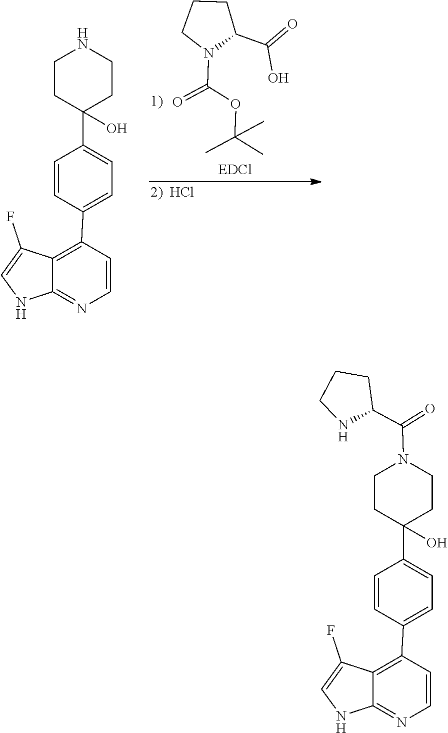 [SMILES: OC1(c2ccc(-c3ccnc4[nH]cc(F)c34)cc2)CCNCC1 ; CC(C)(C)OC(=O)N1CCC[C@@H]1C(=O)O ; O=C([C@H]1CCCN1)N1CCC(O)(c2ccc(-c3ccnc4[nH]cc(F)c34)cc2)CC1]

A suspension containing (R)-1-(tert-butoxycarbonyl)pyrrolidine-2-carboxylic acid (6.91 mg, 0.032 mmol, N-Boc-L-proline), hydroxybenzotriazole (7.38 mg, 0.048 mmol, HOBT) in dichloromethane (1.00 mL) was treated with SiliaBond ethyl-dimethylamino carbodiimide (50 mg, 0.053 mmol) at room temperature for 10 min. Then, 4-(4-(3-fluoro-1H-pyrrolo[2,3-b]pyridin-4-yl)phenyl)piperidin-4-ol (10 mg, 0.032 mmol) was added. The reaction mixture was stirred at 23° C. for 20 hr. The reaction mixture was filtered through a nylon syringe filter and the solvent removed by rotary evaporation. The crude material was dissolved into dioxane (0.500 mL). The resulting solution was treated with 4 M hydrochloric acid solution in dioxane (250 μL, 1.000 mmol) at 23° C. for 1.5 hr. The solvent was then removed by rotary evaporation. The residue was dissolved in methanol (0.5 mL) and purified by reverse phase HPLC to provide the final product as the di-formic acid salt. LC-MS (m/z [M+H)]⁺): 409.2

### Compounds 143-145

Compounds 141-145 were synthesized in an analogous manner to that of Compound 142, and are shown in Table 24.

TABLE 24

| | | | | LCMS |
|---|---|---|---|---|
| Compound | Structure | IUPAC name | ¹H NMR | m/z |
| 143 | 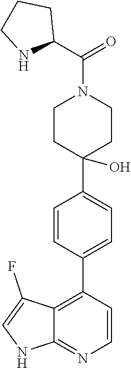 [SMILES: O=C([C@@H]1CCCN1)N1CCC(O)(c2ccc(-c3ccnc4[nH]cc(F)c34)cc2)CC1] | (S)-3-fluoro-4-(4- (4-hydroxy-1- prolylpiperidin-4- yl)phenyl)-1H- pyrrolo[2,3- b]pyridine | | [M + H]⁺ 409.2 |
| 144 | 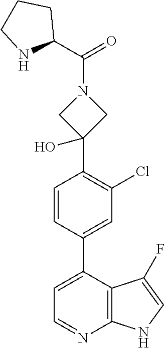 [SMILES: O=C([C@@H]1CCCN1)N1CC(O)(c2ccc(-c3ccnc4[nH]cc(F)c34)cc2Cl)C1] | (S)-4-(3-chloro-4- (3-hydroxy-1- prolylazetidin-3- yl)phenyl)-3-fluoro- 1H-pyrrolo[2,3- b]pyridine | ¹H NMR (400 MHz, DMSO-d₆) δ 11.75 (s, 1H), 8.35 (d, J = 4.9 Hz, 1H), 8.19 (s, 1H), 7.80 − 7.74 (m, 1H), 7.71 − 7.62 (m, 2H), 7.59 (s, 1H), 7.21 (d, J = 4.9 Hz, 1H), 4.84 (dd, J = 38.4, 9.8 Hz, 1H), 4.56 (t, J = 10.2 Hz, 1H), 4.42 (dd, J = 32.6, 9.7 Hz, 1H), 4.19 − 4.03 (m, 1H), 3.90 − 3.77 (m, 1H), 3.02 (dd, J = 11.0, 5.9 Hz, 1H), 2.93 − 2.77 (m, 1H), 1.79 − 1.61 (m, 2H). | [M + H]⁺ 415.2 |
| 145 | 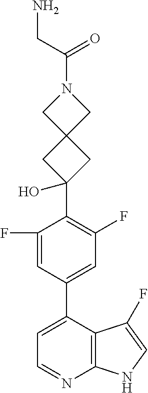 [SMILES: NCC(=O)N1CC2(C1)CC(O)(c1c(F)cc(-c3ccnc4[nH]cc(F)c34)cc1F)C2] | 2-amino-1-(6-(2,6- difluoro-4-(3- fluoro-1H- pyrrolo[2,3- b]pyridin-4- yl)phenyl)-6- hydroxy-2- azaspiro[3.3]heptan- 2-yl)ethan-1-one | ¹H NMR (400 MHz, DMSO-d₆) δ 11.77 (s, 1H), 8.34 (d, J = 4.9 Hz, 1H), 7.59 (s, 1H), 7.33 (d, J = 8.8 Hz, 2H), 7.20 (d, J = 4.9 Hz, 1H), 5.88 (s, 1H), 4.34 (s, 1H), 4.09 (d, J = 17.8 Hz, 2H), 3.86 (s, 1H), 3.41 (d, J = 26.8 Hz, 2H), 2.80 (s, 2H), 2.61 (d, J = 11.6 Hz, 2H). | [M + H]⁺ 417.2 |

### Synthesis of Compounds 146-149

Compounds 146-149 can be prepared in accordance with Steps I-Q of the general synthetic protocols of Example 2.

### Compound 146

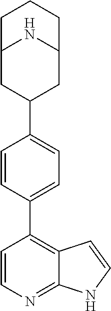 [SMILES: c1cc(-c2ccc(C3CC4CCCC(C3)N4)cc2)c2cc[nH]c2n1]

### 4-(4-(9-azabicyclo[3.3.1]nonan-3-yl)phenyl)-1H-pyrrolo[2,3-b]pyridine

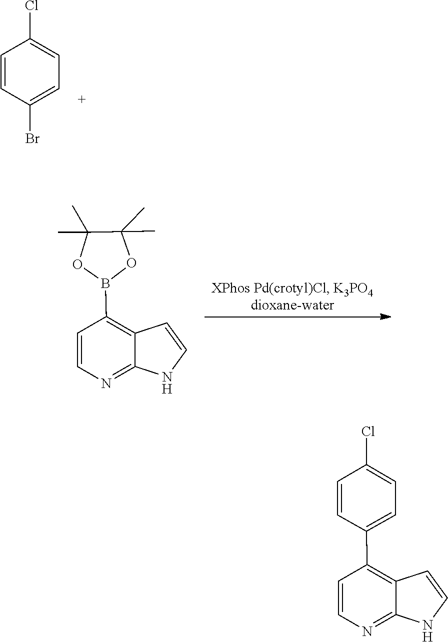 [SMILES: Clc1ccc(Br)cc1 ; CC1(C)OB(c2ccnc3[nH]ccc23)OC1(C)C ; Clc1ccc(-c2ccnc3[nH]ccc23)cc1]

### Step I: 4-(4-chlorophenyl)-1H-pyrrolo[2,3-b]pyridine

A microwave vial was charged with 1-bromo-4-chlorobenzene (1.426 g, 7.45 mmol), 4-(4,4,5,5-tetramethyl-1,3,2-dioxaborolan-2-yl)-1H-pyrrolo[2,3-b]pyridine (2.00 g, 8.19 mmol) and XPhos Pd(crotyl)Cl (0.251 g, 0.372 mmol). It was sealed, then purged with nitrogen twice. Then dioxane (22.4 mL) and 2 M aqueous potassium phosphate tribasic (5.60 mL, 11.2 mmol) were added. The content of the reaction vial was again purged with nitrogen twice. The resulting mixture was stirred at 100° C. for 4 h. The reaction mixture was allowed to cool down to room temperature and was then partitioned between 25 mL of water and 25 mL of ethyl acetate. The layers were separated; the aqueous layer was extracted with two 25-mL portions of ethyl acetate. The combined organic layer was washed with 25 mL of brine, was then dried (sodium sulfate), and was then concentrated under diminished pressure. The residue was applied to a silica gel column; eluting with 100:0→0:100 dichloromethane-ethyl acetate afforded the title compound; ¹H NMR (400 MHz, Chloroform-d) δ 10.58 (s, 1H), 8.39 (d, J=5.0 Hz, 1H), 7.72-7.67 (m, 2H), 7.53-7.45 (m, 2H), 7.44-7.42 (m, 1H), 7.16 (d, J=5.0 Hz, 1H), 6.67 (d, J=3.5 Hz, 1H).

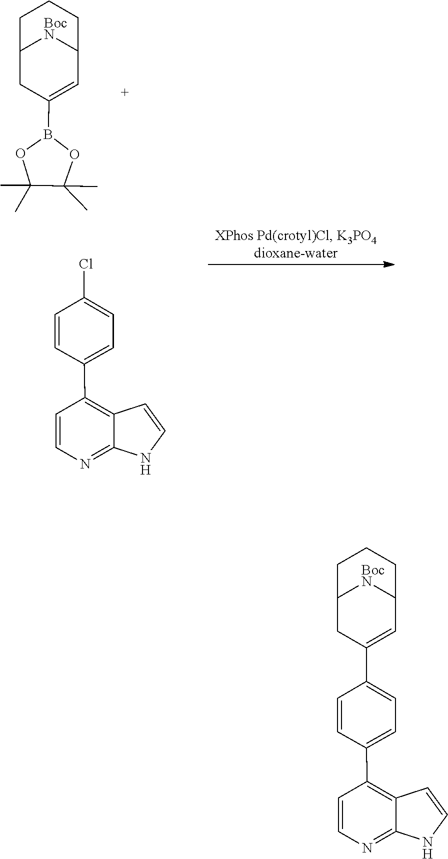

### Step J: tert-butyl 3-(4-(1H-pyrrolo[2,3-b]pyridin-4-yl)phenyl)-9-azabicyclo[3.3.1]non-2-ene-9-carboxylate

A microwave vial was charged with tert-butyl 3-(4,4,5,5-tetramethyl-1,3,2-dioxaborolan-2-yl)-9-azabicyclo[3.3.1]non-2-ene-9-carboxylate (168 mg, 0.481 mmol), 4-(4-chlorophenyl)-1H-pyrrolo[2,3-b]pyridine (100 mg, 0.437 mmol) and XPhosPd(crotyl)Cl (14.7 mg, 0.022 mmol). The vial was capped, and its content was purged with nitrogen twice. Then dioxane (1.32 mL) and 2 M aqueous potassium phosphate tribasic (0.330 mL, 0.660 mmol) were added. The content of the reaction vial was again purged with nitrogen twice. The resulting mixture was stirred at 100° C. for 3 h. The reaction mixture was allowed to cool down to room temperature, and was then diluted with 10 mL of ethyl acetate. The reaction mixture was filtered through a pad of celite. The collected solid was washed two 5-mL portions of ethyl acetate. The collected filtrate was then concentrated under diminished pressure. The residue was applied to a silica gel column; eluting with 100:0→0:100 dichloromethane-ethyl acetate afforded the title compound; ¹H NMR (400 MHz, Chloroform-d) δ 10.42 (s, 1H), 8.38 (d, J=5.0 Hz, 1H), 7.79-7.71 (m, 2H), 7.62-7.52 (m, 2H), 7.42 (d, J=3.6 Hz, 1H), 7.20 (d, J=5.1 Hz, 1H), 6.73 (d, J=3.5 Hz, 1H), 6.23 (dd, J=19.4, 5.7 Hz, 1H), 4.94-4.73 (m, 1H), 4.70-4.52 (m, 1H), 2.99 (ddd, J=33.8, 17.6, 7.5 Hz, 1H), 2.39 (dd, J=17.7, 4.9 Hz, 1H), 2.22 (s, 2H), 1.81 (q, J=15.1, 12.9 Hz, 2H), 1.69-1.52 (m, 2H), 1.49 (d, J=1.5 Hz, 9H).

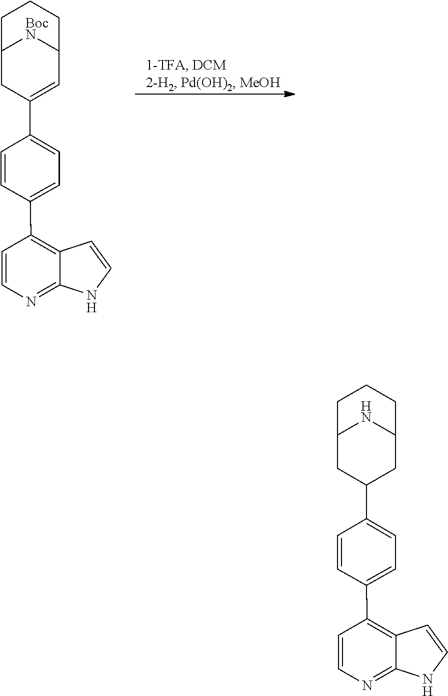 [SMILES: CCCCC1C=C(c2ccc(-c3ccnc4[nH]ccc34)cc2)CCN1C(=O)OC(C)(C)C ; CCCCC1CC(c2ccc(-c3ccnc4[nH]ccc34)cc2)CCN1]

### Step K: 4-(4-(9-azabicyclo[3.3.1]nonan-3-yl)phenyl)-1H-pyrrolo[2,3-b]pyridine

A solution containing tert-butyl 3-(4-(1H-pyrrolo[2,3-b]pyridin-4-yl)phenyl)-9-azabicyclo[3.3.1]non-2-ene-9-carboxylate (0.060 g, 0.144 mmol) in dioxane (0.720 mL) was treated with hydrochloric acid (4 M solution in dioxane) (0.720 ml, 2.80 mmol) at 23° C. for 4 h. The reaction mixture was concentrated under diminished pressure. The residue was dissolved in methanol (2.20 mL) and the resulting solution was stirred in the presence of Pearlman's catalyst (1.00 mg, 7.22 μmol) under an atmosphere of hydrogen (1 atm) at 23° C. for 3 h. The reaction mixture was diluted with 5 mL of methanol, then a small portion of celite was added in order to trap the palladium catalyst. The resulting suspension was filtered, and the collected filtrate was concentrated under diminished pressure. The residue was purified by HPLC to afford the title compound; ¹H NMR (400 MHz, DMSO-d₆) δ 11.86 (s, 1H), 8.51 (d, J=12.7 Hz, 1H), 8.29 (d, J=5.0 Hz, 1H), 7.79-7.73 (m, 2H), 7.56 (dd, J=3.5, 2.5 Hz, 1H), 7.54-7.49 (m, 2H), 7.19 (d, J=5.0 Hz, 1H), 6.62 (dd, J=3.5, 1.9 Hz, 1H), 3.89-3.79 (m, 2H), 3.17-3.00 (m, 1H), 2.37-2.21 (m, 2H), 2.19-2.04 (m, 1H), 1.91-1.69 (m, 4H), 1.68-1.49 (m, 3H).

### Compound 147

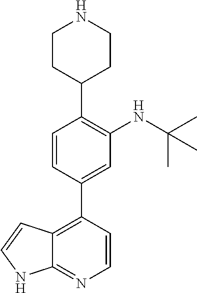 [SMILES: CC(C)(C)Nc1cc(-c2ccnc3[nH]ccc23)ccc1C1CCNCC1]

### 4-(3-chloro-4-(piperidin-4-yl)phenyl)-3-fluoro-1H-pyrrolo[2,3-b]pyridine

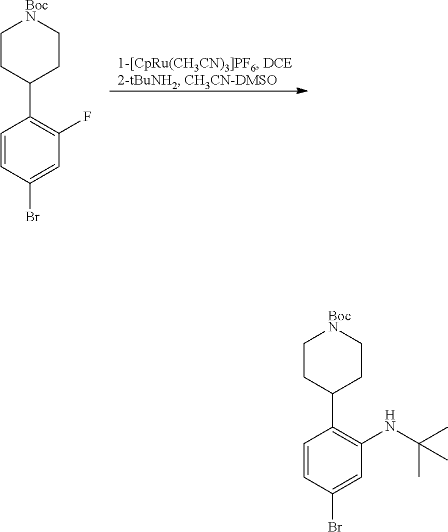 [SMILES: CC(C)(C)OC(=O)N1CCC(c2ccc(Br)cc2F)CC1 ; CC(C)(C)Nc1cc(Br)ccc1C1CCN(C(=O)OC(C)(C)C)CC1]

### Step L: tert-butyl 4-(4-bromo-2-(tert-butylamino)phenyl)piperidine-1-carboxylate

A mixture containing tert-butyl 4-(4-bromo-2-fluorophenyl)piperidine-1-carboxylate (0.100 g, 0.279 mmol) and tris(acetonitrile)cyclopentadienylruthenium(II) hexafluorophosphate (0.121 g, 0.279 mmol) in DCM (2.80 mL) was stirred at 90° C. for 1.5 h. The reaction mixture was concentrated under diminished pressure. Half of the obtained residue was dissolved in 1:1 acetonitrile-dimethylsulfoxide (1.40 mL), then tert-butylamine (15 μL, 0.140 mmol) was added and the reaction mixture was heated in the microwave reactor at 130° C. for 45 min. The reaction mixture was allowed to cool down to room temperature and was then partitioned between 5 mL of water and 5 mL of ethyl acetate. The layers were separated; the aqueous layer was extracted with two 10-mL portions of ethyl acetate. The combined organic layer was washed with 10 mL of brine, was then dried (sodium sulfate), and was then concentrated under diminished pressure. The residue was purified by HPLC to afford the title compound.

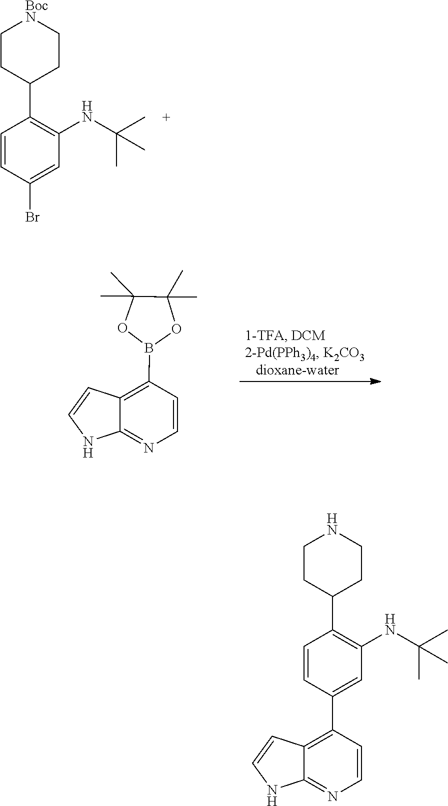 [SMILES: CC(C)(C)Nc1cc(Br)ccc1C1CCN(C(=O)OC(C)(C)C)CC1 ; CC1(C)OB(c2ccnc3[nH]ccc23)OC1(C)C ; CC(C)(C)Nc1cc(-c2ccnc3[nH]ccc23)ccc1C1CCNCC1]

### Step M: N-(tert-butyl)-2-(piperidin-4-yl)-5-(1H-pyrrolo[2,3-b]pyridin-4-yl)aniline

A solution containing tert-butyl 4-(4-bromo-2-(tert-butylamino)phenyl)piperidine-1-carboxylate (0.015 g, 0.036 mmol) in DCM (0.360 mL) was treated with trifluoroacetic acid (42.0 μL, 0.545 mmol) at 23° C. for 2 h. The reaction mixture was concentrated under diminished pressure. The residue was dissolved in 1:1 dioxane-water (1.00 mL), then potassium carbonate (0.100 g, 0.724 mmol) was added, followed by 4-(4,4,5,5-tetramethyl-1,3,2-dioxaborolan-2-yl)-1H-pyrrolo[2,3-b]pyridine (0.030 g, 0.123 mmol) and tetrakis(triphenylphosphine)palladium(0) (0.010 g, 8.65 μmol). The resulting mixture was heated in the microwave reactor at 150° C. for 30 min. The reaction mixture was allowed to cool down to room temperature, and was then concentrated under diminished pressure. Purification of the obtained residue by HPLC afforded the title compound.

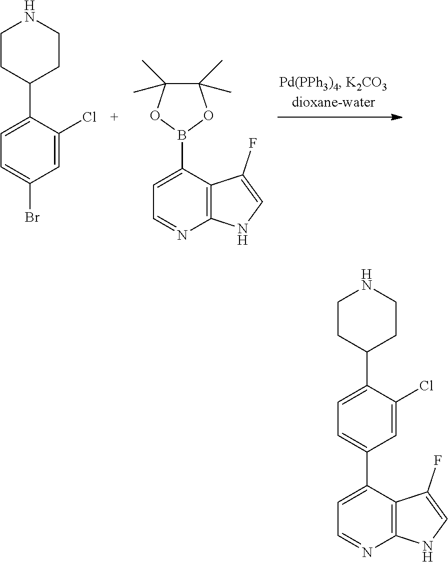 [SMILES: Clc1cc(Br)ccc1C1CCNCC1 ; CC1(C)OB(c2ccnc3[nH]cc(F)c23)OC1(C)C ; Fc1c[nH]c2nccc(-c3ccc(C4CCNCC4)c(Cl)c3)c12]

### Step N: 4-(3-chloro-4-(piperidin-4-yl)phenyl)-3-fluoro-1H-pyrrolo[2,3-b]pyridine

A microwave vial was charged with 3-fluoro-4-(4,4,5,5-tetramethyl-1,3,2-dioxaborolan-2-yl)-1H-pyrrolo[2,3-b]pyridine (0.333 g, 1.27 mmol), 4-(4-bromo-2-chlorophenyl)piperidine (0.317 g, 1.15 mmol) and palladium tetrakistriphenylphosphine (0.067 g, 0.058 mmol). The content of the reaction vessel was purged with nitrogen. Then, dioxane (3.60 mL) was added, followed by 2 M aqueous potassium carbonate (0.870 mL, 1.74 mmol). The content of the reaction vessel was purged with nitrogen. The reaction mixture was heated at 120° C. for 45 min, was then allowed to cool down to room temperature, and was then diluted with 25 mL of methanol. The resulting suspension was filtered through a pad of celite. The collected filtrate was concentrated under diminished pressure. The residue was applied to a C-18 column; eluting with 100:0→50:50 water-acetonitrile (+0.1% formic acid) afforded the title compound; ¹H NMR (400 MHz, DMSO-d₆) δ 11.72 (s, 1H), 8.41 (s, 1H), 8.36-8.29 (m, 1H), 7.71 (dd, J=2.9, 1.9 Hz, 1H), 7.64 (dt, J=8.1, 2.4 Hz, 1H), 7.55 (d, J=2.0 Hz, 1H), 7.49 (d, J=8.2 Hz, 1H), 7.18 (d, J=5.0 Hz, 1H), 3.34-3.19 (m, 3H), 2.92 (td, J=12.3, 3.3 Hz, 2H), 1.95-1.76 (m, 4H).

### Compound 148

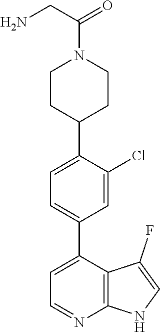 [SMILES: NCC(=O)N1CCC(c2ccc(-c3ccnc4[nH]cc(F)c34)cc2Cl)CC1]

### 2-amino-1-(4-(2-chloro-4-(3-fluoro-1H-pyrrolo[2,3-b]pyridin-4-yl)phenyl)piperidin-1-yl)ethan-1-one

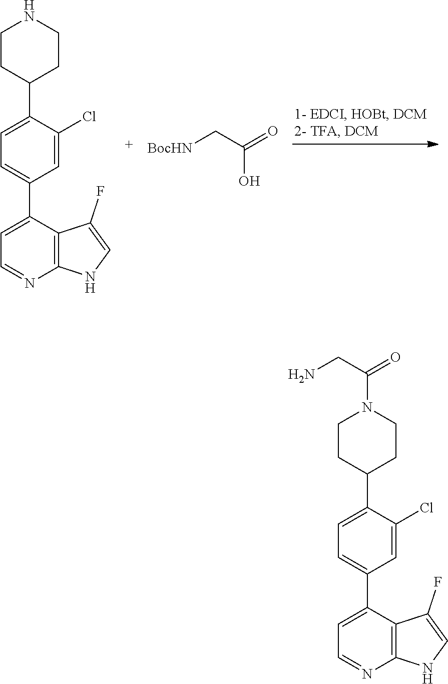 [SMILES: Fc1c[nH]c2nccc(-c3ccc(C4CCNCC4)c(Cl)c3)c12 ; CC(C)(C)OC(=O)NCC(=O)O ; NCC(=O)N1CCC(c2ccc(-c3ccnc4[nH]cc(F)c34)cc2Cl)CC1]

Step O: A microwave vial was charged with N-Boc-glycine (17.5 mg, 0.100 mmol), ethyl-dimethylamino carbodiimide (170 mg, 0.179 mmol), HOBt (20.9 mg, 0.136 mmol), and DCM (1.80 mL). To the resulting suspension was added a solution containing 4-(3-chloro-4-(piperidin-4-yl)phenyl)-3-fluoro-1H-pyrrolo[2,3-b]pyridine (30 mg, 0.091 mmol) in 1.00 mL of DCM. The reaction mixture was stirred at 23° C. for 2 hr. The reaction mixture was partitioned with 2 mL of 0.5 M aq. NaOH. The layers were separated, the organic layer was dried with sodium sulfate, and was then concentrated under diminished pressure. The residue was dissolved into 0.340 mL of dioxane, and the resulting solution was treated with 4 M hydrochloric acid solution in dioxane (340 μL, 1.36 mmol) at 23° C. for 4 hr. The reaction mixture was concentrated and the resulting residue was purified by HPLC to afford the title compound; ¹H NMR (400 MHz, DMSO-d₆) δ 11.71 (s, 1H), 8.32 (d, J=4.9 Hz, 1H), 8.25 (s, 1H), 7.71 (dd, J=2.8, 1.9 Hz, 1H), 7.62 (dt, J=8.1, 2.6 Hz, 1H), 7.56 (d, J=2.0 Hz, 1H), 7.50 (d, J=8.2 Hz, 1H), 7.19 (d, J=4.9 Hz, 1H), 4.58 (d, J=12.9 Hz, 1H), 3.87 (d, J=13.5 Hz, 1H), 3.73-3.56 (m, 2H), 3.17 (t, J=13.1 Hz, 2H), 2.77 (t, J=12.6 Hz, 1H), 1.86 (d, J=13.0 Hz, 2H), 1.78-1.62 (m, 1H), 1.61-1.44 (m, 1H).

### Compound 149

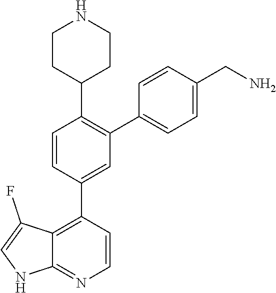 [SMILES: NCc1ccc(-c2cc(-c3ccnc4[nH]cc(F)c34)ccc2C2CCNCC2)cc1]

### (5′-(3-fluoro-1H-pyrrolo[2,3-b]pyridin-4-yl)-2′-(piperidin-4-yl)-[1,1′-biphenyl]-4-yl)methanamine

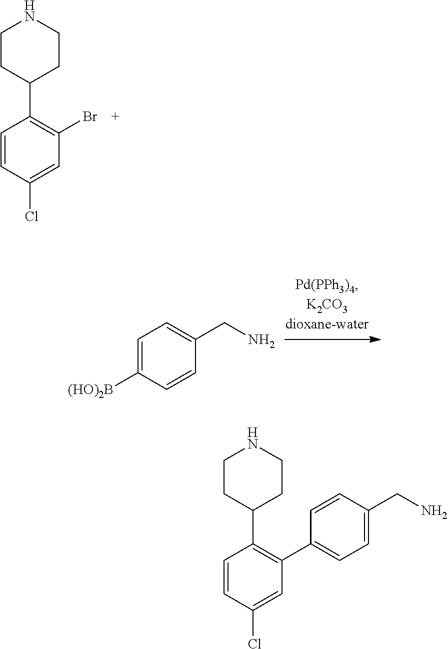 [SMILES: Clc1ccc(C2CCNCC2)c(Br)c1 ; NCc1ccc(B(O)O)cc1 ; NCc1ccc(-c2cc(Cl)ccc2C2CCNCC2)cc1]

### Step P: (5′-chloro-2′-(piperidin-4-yl)-[1,1′-biphenyl]-4-yl)methanamine

A microwave vial was charged with 4-(2-bromo-4-chlorophenyl)piperidine (0.300 g, 1.09 mmol), (4-(aminomethyl)phenyl)boronic acid (0.165 g, 1.09 mmol), potassium carbonate (0.500 g, 3.62 mmol), and palladium tetrakistriphenylphosphine (0.030 g, 0.026 mmol). The content of the reaction vessel was purged with nitrogen. Then, 3:1 dioxane-water (16.0 mL) was added. The content of the reaction vessel was purged with nitrogen. The reaction mixture was heated at 150° C. for 30 min, was then allowed to cool down to room temperature, and was then diluted with 25 mL of methanol. The resulting suspension was filtered through a pad of celite. The collected filtrate was concentrated under diminished pressure. The residue was applied to a C-18 column; eluting with 100:0→50:50 water-acetonitrile (+0.1% formic acid) afforded the title compound.

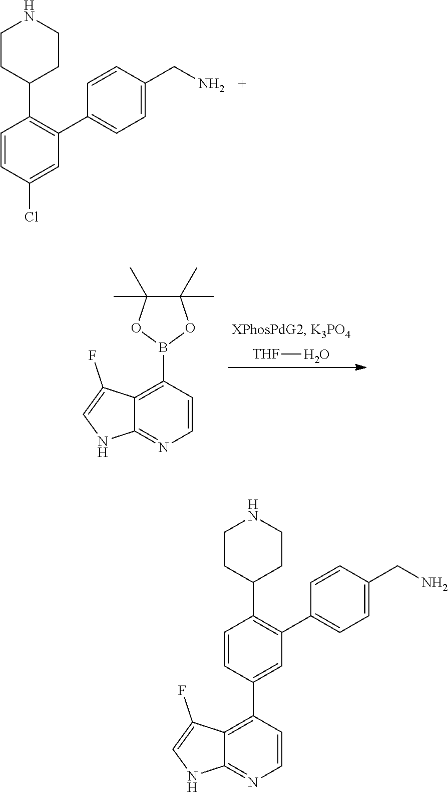

### Step Q: (5′-(3-fluoro-1H-pyrrolo[2,3-b]pyridin-4-yl)-2′-(piperidin-4-yl)-[1,1′-biphenyl]-4-yl)methanamine

A microwave vial was charged with (5′-chloro-2′-(piperidin-4-yl)-[1,1′-biphenyl]-4-yl)methanamine (0.050 g, 0.166 mmol), 3-fluoro-4-(4,4,5,5-tetramethyl-1,3,2-dioxaborolan-2-yl)-1H-pyrrolo[2,3-b]pyridine (0.050 g, 0.191 mmol), potassium phosphate (0.150 g, 0.707 mmol), and chloro(2-dicyclohexylphosphino-2′,4′,6′-triisopropyl-1,1′-biphenyl)[2-(2′-amino-1,1′-biphenyl)]palladium(II) (0.100 g, 0.127 mmol). The content of the reaction vessel was purged with nitrogen. Then, 1:1 tetrahydrofuran-water (2.00 mL) was added. The content of the reaction vessel was purged with nitrogen. The reaction mixture was heated at 100° C. for 45 min, was then allowed to cool down to room temperature, and was then diluted with 15 mL of methanol. The resulting suspension was filtered through a pad of celite. The collected filtrate was concentrated under diminished pressure. The residue was applied to a C-18 column; eluting with 100:0→50:50 water-acetonitrile (+0.1% formic acid) afforded the title compound. ¹H NMR (400 Hz, DMSO-d₆) δ 11.69 (d, J=2.6 Hz, 1H), 8.55 (s, 1H), 8.32 (d, J=4.9 Hz, 1H), 8.20 (s, 3H), 7.76 (d, J=8.3 Hz, 1H), 7.63-7.37 (m, 7H), 7.21 (d, J=5.0 Hz, 1H), 4.16-4.09 (m, 2H), 3.35 (d, J=12.3 Hz, 2H), 3.02-2.87 (m, 1H), 2.83-2.70 (m, 2H), 1.97-1.84 (i, 4H). LC-MS (m/z [M+H)]⁺): 401.2

### Compounds 150-188

Compounds 150-188 were all prepared using the synthetic procedures described above, substituting the appropriate reactants and reagents that were prepared as described herein or are commercially available, and are shown in Table 25.

TABLE 25

| | | | | LC/MS |
|---|---|---|---|---|
| | | | | m/z |
| Compound | Structure | IUPAC Name | ¹H NMR | [M + H]⁺ |
| 150 | 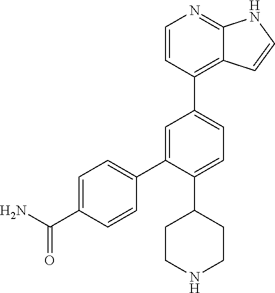 [SMILES: NC(=O)c1ccc(-c2cc(-c3ccnc4[nH]ccc34)ccc2C2CCNCC2)cc1] | 2′-(piperidin-4- yl)-5′-(1H- pyrrolo[2,3- b]pyridin-4-yl)- [1,1′-biphenyl]- 4-carboxamide | ¹H NMR (400 MHz, DMSO-d₆) δ 11.84 (s, 1H), 8.53 (d, J = 11.6 Hz, 1H), 8.28 (d, J = 5.0 Hz, 1H), 8.06 (s, 1H), 7.99 (d, J = 8.0 Hz, 2H), 7.91 − 7.81 (m, 1H), 7.63 − 7.48 (m, 5H), 7.44 (s, 1H), 7.25 (d, J = 5.0 Hz, 1H), 6.64 (dd, J = 3.4, 1.8 Hz, 1H), 3.33 (d, J = 12.4 Hz, 2H), 2.99 − 2.90 (m, 1H), 2.92 − 2.77 (m, 2H), 1.94 − 1.87 (m, 4H). | 397.2 |
| 151 | 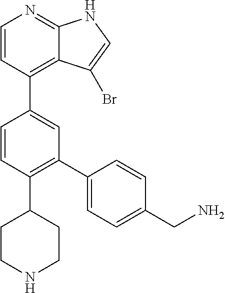 [SMILES: NCc1ccc(-c2cc(-c3ccnc4[nH]cc(Br)c34)ccc2C2CCNCC2)cc1] | (5′-(3-bromo- 1H-pyrrolo[2,3- b]pyridin-4-yl)- 2′-(piperidin-4- yl)-[1,1′- biphenyl]-4- yl)methanamine | | 461.1 |
| 152 | 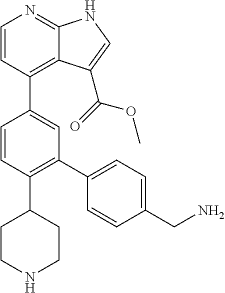 [SMILES: COC(=O)c1c[nH]c2nccc(-c3ccc(C4CCNCC4)c(-c4ccc(CN)cc4)c3)c12] | methyl 4-(4′- (aminomethyl)- 6-(piperidin-4- yl)-[1,1′- biphenyl]-3-yl)- 1H-pyrrolo[2,3- b]pyridine-3- carboxylate | ¹H NMR (400 MHz, DMSO-d₆) δ 12.62 (s, 1H), 8.60 (s, 1H), 8.36 (d, J = 4.9 Hz, 1H), 8.20 (d, J = 3.5 Hz, 4H), 7.53 (dd, J = 12.9, 8.0 Hz, 4H), 7.44 (t, J = 8.2 Hz, 3H), 7.19 (d, J = 4.9 Hz, 1H), 7.08 (d, J = 1.8 Hz, 1H), 4.14 − 4.07 (m, 2H), 3.35 (d, J = 12.3 Hz, 2H), 3.27 (s, 3H), 2.98 − 2.88 (m, 1H), 2.83 − 2.66 (m, 2H), 2.01 − 1.84 (m, 4H). | 441.2 |
| 153 | 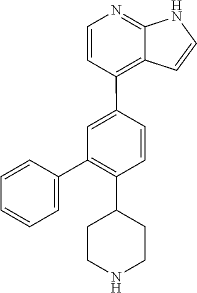 [SMILES: c1ccc(-c2cc(-c3ccnc4[nH]ccc34)ccc2C2CCNCC2)cc1] | 4-(6-(piperidin- 4-yl)-[1,1′- biphenyl]-3-yl)- 1H-pyrrolo[2,3- b]pyridine | ¹H NMR (400 MHz, DMSO-d₆) δ 11.85 (s, 1H), 8.58 (d, J = 10.4 Hz, 1H), 8.37 (d, J = 11.4 Hz, 1H), 8.28 (d, J = 5.0 Hz, 1H), 7.85 (dd, J = 8.2, 2.0 Hz, 1H), 7.62 − 7.40 (m, 8H), 7.24 (d, J = 5.0 Hz, 1H), 6.63 (dd, J = 3.6, 1.8 Hz, 1H), 3.33 (d, J = 12.4 Hz, 2H), 3.08 − 2.93 (m, 1H), 2.82 (d, J = 11.8 Hz, 2H), 2.10 − 1.67 (m, 4H). | 354.2 |
| 154 | 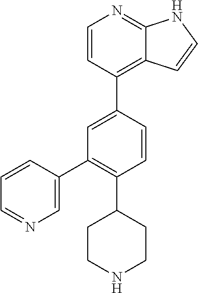 [SMILES: c1cncc(-c2cc(-c3ccnc4[nH]ccc34)ccc2C2CCNCC2)c1] | 4-(4-(piperidin- 4-yl)-3-(pyridin- 3-yl)phenyl)- 1H-pyrrolo[2,3- b]pyridine | ¹H NMR (400 MHz, DMSO-d₆) δ 11.89 (s, 1H), 8.78 − 8.70 (m, 2H), 8.58 (d, J = 11.5 Hz, 1H), 8.35 (d, J = 12.8 Hz, 1H), 8.30 (d, J = 5.0 Hz, 1H), 8.04 (d, J = 7.9 Hz, 1H), 7.93 (dd, J = 8.1, 2.0 Hz, 1H), 7.68 − 7.60 (m, 2H), 7.60 − 7.54 (m, 2H), 7.28 (d, J = 5.0 Hz, 1H), 6.68 (dd, J = 3.6, 1.8 Hz, 1H), 3.33 (d, J = 12.3 Hz, 2H), 3.05 − 2.69 (m, 4H), 1.96 − 1.86 (m, 4H). | 355.2 |
| 155 | 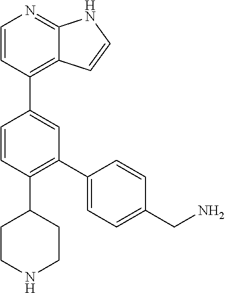 [SMILES: NCc1ccc(-c2cc(-c3ccnc4[nH]ccc34)ccc2C2CCNCC2)cc1] | (2′-(piperidin-4- yl)-5′-(1H- pyrrolo[2,3- b]pyridin-4-yl)- [1,1′-biphenyl]- 4- yl)methanamine | ¹H NMR (400 MHz, DMSO-d₆) δ 11.84 (s, 1H), 8.63 (bs, 1H), 8.41 (bs, 1H), 8.28 (d, J = 5.0 Hz, 1H), 8.25 (bs, 3H), 7.86 (dd, J = 8.2, 2.0 Hz, 1H), 7.65 − 7.46 (m, 7H), 7.23 (dd, J = 5.1, 1.8 Hz, 1H), 6.61 (dd, J = 3.6, 1.8 Hz, 1H), 4.13 (q, J = 5.8 Hz, 2H), 3.35 (d, J = 12.5 Hz, 2H), 3.02 − 2.87 (m, 1H), 2.83 − 2.70 (m, 2H), 2.0 − 1.86 (m, 4H). | 383.2 |
| 156 | 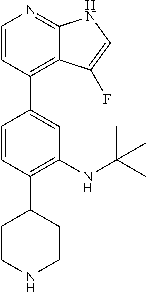 [SMILES: CC(C)(C)Nc1cc(-c2ccnc3[nH]cc(F)c23)ccc1C1CCNCC1] | N-(tert-butyl)-5- (3-fluoro-1H- pyrrolo[2,3- b]pyridin-4-yl)- 2-(piperidin-4- yl)aniline | ¹H NMR (400 MHz, DMSO-d₆) δ 11.70 (s, 1H), 8.61 (s, 1H), 8.33 (d, J = 4.8 Hz, 1H), 7.55 (s, 1H), 7.32 (s, 3H), 7.11 (d, J = 4.9 Hz, 1H), 3.43 (d, J = 12.3 Hz, 2H), 3.20 − 2.97 (m, 3H), 1.99 − 1.75 (m, 4H), 1.39 (s, 9H). | 367.3 |
| 157 | 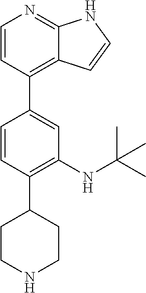 [SMILES: CC(C)(C)Nc1cc(-c2ccnc3[nH]ccc23)ccc1C1CCNCC1] | N-(tert-butyl)-2- (piperidin-4-yl)- 5-(1H- pyrrolo[2,3- b]pyridin-4- yl)aniline | ¹H NMR (400 MHz, DMSO-d₆) δ 11.89 (s, 1H), 8.61 (s, 1H), 8.34 (dd, J = 25.0, 4.9 Hz, 1H), 7.58 (d, J = 10.6 Hz, 1H), 7.18 (d, J = 5.0 Hz, 1H), 6.64 (s, 1H), 3.43 (d, J = 12.3 Hz, 2H), 3.22 − 2.96 (m, 3H), 1.96 − 1.77 (m, 3H), 1.41 (s, 9H). | 349.2 |
| 158 | 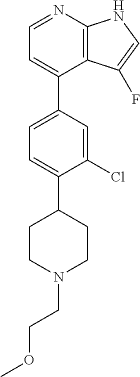 [SMILES: COCCN1CCC(c2ccc(-c3ccnc4[nH]cc(F)c34)cc2Cl)CC1] | 4-(3-chloro-4- (1-(2- methoxyethyl) piperidin-4- yl)phenyl)-3- fluoro-1H- pyrrolo[2,3- b]pyridine | | 388.2 |
| 159 | 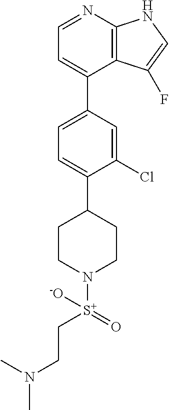 | 2-((4-(2-chloro- 4-(3-fluoro-1H- pyrrolo[2,3- b]pyridin-4- yl)phenyl) piperidin-1- yl)sulfonyl)- N,N- dimethylethan- 1-amine | | 465.1 |
| 160 | 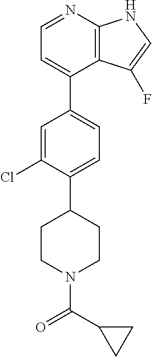 [SMILES: O=C(C1CC1)N1CCC(c2ccc(-c3ccnc4[nH]cc(F)c34)cc2Cl)CC1] | (4-(2-chloro-4- (3-fluoro-1H- pyrrolo[2,3- b]pyridin-4- yl)phenyl) piperidin-1- yl)(cyclopropyl) methanone | | 398.1 |
| 161 | 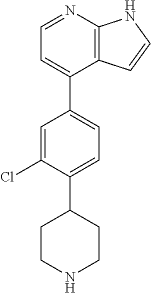 [SMILES: Clc1cc(-c2ccnc3[nH]ccc23)ccc1C1CCNCC1] | 4-(3-chloro-4- (piperidin-4- yl)phenyl)-1H- pyrrolo[2,3- b]pyridine | ¹H NMR (400 MHz, DMSO-d₆) δ 11.86 (s, 1H), 8.38 (s, 1H), 8.28 (d, J = 5.0 Hz, 1H), 7.83 − 7.72 (m, 2H), 7.56 (dd, J = 3.5, 2.2 Hz, 1H), 7.52 (d, J = 8.0 Hz, 1H), 7.20 (d, J = 5.0 Hz, 1H), 6.60 (dd, J = 3.6, 1.5 Hz, 1H), 3.30 − 3.17 (m, 3H), 2.89 (td, J = 12.4, 2.9 Hz, 2H), 1.93 − 1.70 (m, 4H). | 312.1 |
| 162 | 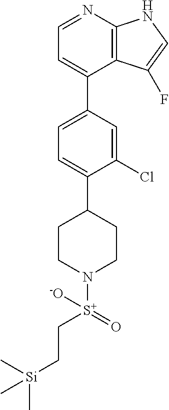 | 4-(3-chloro-4- (1-((2- (trimethylsilyl) ethyl)sulfonyl) piperidin-4- yl)phenyl)-3- fluoro-1H- pyrrolo[2,3- b]pyridine | | 494.1 |
| 163 | 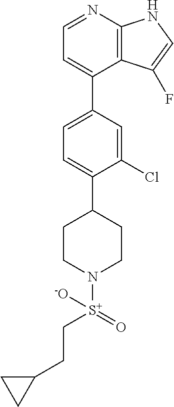 | 4-(3-chloro-4- (1-((2- cyclopropylethyl) sulfonyl) piperidin-4- yl)phenyl)-3- fluoro-1H- pyrrolo[2,3- b]pyridine | | 462.1 |
| 164 | 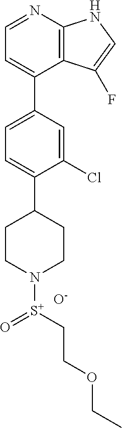 | 4-(3-chloro-4- (1-((2- ethoxyethyl) sulfonyl)piperidin- 4-yl)phenyl)-3- fluoro-1H- pyrrolo[2,3- b]pyridine | | 466.1 |
| 165 | 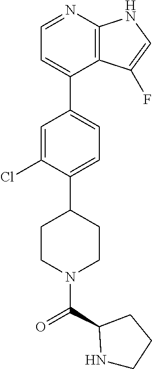 [SMILES: O=C([C@H]1CCCN1)N1CCC(c2ccc(-c3ccnc4[nH]cc(F)c34)cc2Cl)CC1] | (R)-4-(3-chloro- 4-(1- prolylpiperidin- 4-yl)phenyl)-3- fluoro-1H- pyrrolo[2,3- b]pyridine | | 427.1 |
| 166 | 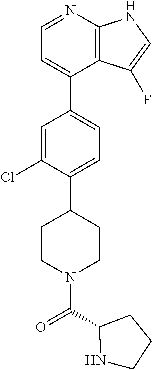 [SMILES: O=C([C@@H]1CCCN1)N1CCC(c2ccc(-c3ccnc4[nH]cc(F)c34)cc2Cl)CC1] | (S)-4-(3-chloro- 4-(1- prolylpiperidin- 4-yl)phenyl)-3- fluoro-1H- pyrrolo[2,3- b]pyridine | | 427.1 |
| 167 | 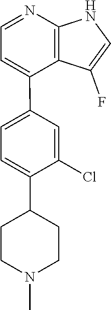 [SMILES: CN1CCC(c2ccc(-c3ccnc4[nH]cc(F)c34)cc2Cl)CC1] | 4-(3-chloro-4- (1- methylpiperidin- 4-yl)phenyl)-3- fluoro-1H- pyrrolo[2,3- b]pyridine | ¹H NMR (400 MHz, DMSO-d₆) δ 11.71 (s, 1H), 8.32 (d, J = 4.9 Hz, 1H), 7.73 (t, J = 2.4 Hz, 1H), 7.66 (d, J = 8.1 Hz, 1H), 7.56 (t, J = 2.5 Hz, 1H), 7.48 (s, 1H), 7.18 (d, J = 4.9 Hz, 1H), 4.07 (q, J = 5.2 Hz, 1H), 3.16 (d, J = 5.2 Hz, 3H), 3.12 − 3.02 (m, 2H), 2.81 − 2.68 (m, 2H), 2.05 − 1.92 (m, 4H). | 344.1 |
| 168 | 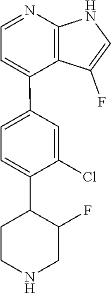 [SMILES: Fc1c[nH]c2nccc(-c3ccc(C4CCNCC4F)c(Cl)c3)c12] | 4-(3-chloro-4- (3- fluoropiperidin- 4-yl)phenyl)-3- fluoro-1H- pyrrolo[2,3- b]pyridine | | 348.1 |
| 169 | 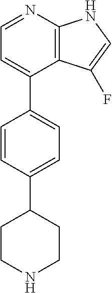 [SMILES: Fc1c[nH]c2nccc(-c3ccc(C4CCNCC4)cc3)c12] | 3-fluoro-4-(4- (piperidin-4- yl)phenyl)-1H- pyrrolo[2,3- b]pyridine | ¹H NMR (400 MHz, DMSO-d₆) δ 11.64 (s, 1H), 8.57 (d, J = 13.5 Hz, 1H), 8.31 (d, J = 4.9 Hz, 1H), 7.64 (dd, J = 8.2, 2.9 Hz, 2H), 7.52 (t, J = 2.4 Hz, 1H), 7.46 − 7.26 (m, 2H), 7.13 (d, J = 4.9 Hz, 1H), 3.41 (d, J = 12.5 Hz, 2H), 3.04 (q, J = 12.0 Hz, 2H), 2.94 (t, J = 12.1 Hz, 1H), 2.02 (d, J = 13.8 Hz, 2H), 1.97 − 1.69 (m, 2H). | 296.1 |
| 170 | 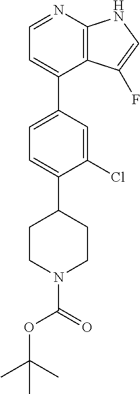 [SMILES: CC(C)(C)OC(=O)N1CCC(c2ccc(-c3ccnc4[nH]cc(F)c34)cc2Cl)CC1] | tert-butyl 4-(2- chloro-4-(3- fluoro-1H- pyrrolo[2,3- b]pyridin-4- yl)phenyl) piperidine-1- carboxylate | | 430.2 |
| 171 | 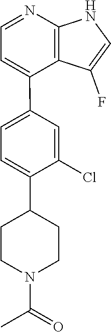 [SMILES: CC(=O)N1CCC(c2ccc(-c3ccnc4[nH]cc(F)c34)cc2Cl)CC1] | 1-(4-(2-chloro- 4-(3-fluoro-1H- pyrrolo[2,3- b]pyridin-4- yl)phenyl)piperidin- 1-yl)ethan- 1-one | | 372.2 |
| 172 | 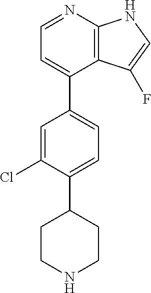 [SMILES: Fc1c[nH]c2nccc(-c3ccc(C4CCNCC4)c(Cl)c3)c12] | 4-(3-chloro-4- (piperidin-4- yl)phenyl)-3- fluoro-1H- pyrrolo[2,3- b]pyridine | ¹H NMR (400 MHz, Chloroform-d) δ 8.37 (d, J = 5.0 Hz, 1H), 7.70 (dd, J = 2.7, 1.9 Hz, 1H), 7.58 (ddd, J = 8.0, 3.1, 1.9 Hz, 1H), 7.42 (d, J = 8.1 Hz, 1H), 7.29 − 7.24 (m, 1H), 7.20 (d, J = 2.3 Hz, 1H), 7.15 − 7.12 (m, 1H), 3.28 − 3.17 (m, 4H), 2.84 (td, J = 12.2, 2.4 Hz, 2H), 1.93 (dt, J = 13.0, 2.8 Hz, 2H), 1.74 − 1.55 (m, 3H). | 330.1 |
| 173 | 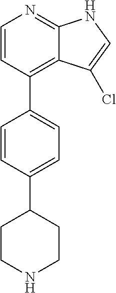 [SMILES: Clc1c[nH]c2nccc(-c3ccc(C4CCNCC4)cc3)c12] | 3-chloro-4-(4- (piperidin-4- yl)phenyl)-1H- pyrrolo[2,3- b]pyridine | ¹H NMR (400 MHz, DMSO-d₆) δ 12.16 (s, 1H), 8.56 (s, 1H), 8.31 (d, J = 4.8 Hz, 1H), 7.70 (d, J = 2.7 Hz, 1H), 7.51 (d, J = 8.1 Hz, 2H), 7.35 (d, J = 8.1 Hz, 2H), 7.03 (d, J = 4.8 Hz, 1H), 3.41 (d, J = 12.5 Hz, 2H), 3.15 − 2.83 (m, 3H), 2.03 (d, J = 13.8 Hz, 2H), 1.91 − 1.73 (m, 2H). | 312.1 |
| 174 | 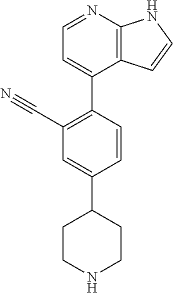 [SMILES: N#Cc1cc(C2CCNCC2)ccc1-c1ccnc2[nH]ccc12] | 5-(piperidin-4- yl)-2-(1H- pyrrolo[2,3- b]pyridin-4- yl)benzonitrile | | 303.2 |
| 175 | 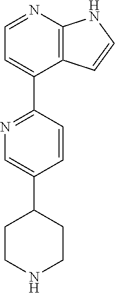 [SMILES: c1cc(-c2ccc(C3CCNCC3)cn2)c2cc[nH]c2n1] | 4-(5-(piperidin- 4-yl)pyridin-2- yl)-1H- pyrrolo[2,3- b]pyridine | ¹H NMR (400 MHz, DMSO-d₆) δ 11.85 (s, 1H), 8.70 (d, J = 2.3 Hz, 1H), 8.64 (bs, 1H), 8.40 (bs, 1H), 8.33 (d, J = 5.0 Hz, 1H), 8.08 (d, J = 8.2 Hz, 1H), 7.83 (dd, J = 8.3, 2.4 Hz, 1H), 7.63 − 7.56 (m, 2H), 6.99 (dd, J = 3.5, 1.9 Hz, 1H), 3.43 (d, J = 12.7 Hz, 2H), 3.15 − 2.95 (m, 3H), 2.05 (d, J = 13.7 Hz, 2H), 1.87 (qd, J = 13.2, 4.0 Hz, 2H). | 279.1 |
| 176 | 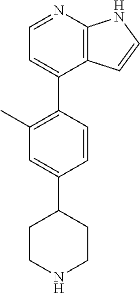 [SMILES: Cc1cc(C2CCNCC2)ccc1-c1ccnc2[nH]ccc12] | 4-(2-methyl-4- (piperidin-4- yl)phenyl)-1H- pyrrolo[2,3- b]pyridine | ¹H NMR (400 MHz, DMSO-d₆) δ 11.97 (s, 1H), 8.39 − 8.21 (m, 1H), 7.66 (d, J = 1.8 Hz, 1H), 7.59 − 7.50 (m, 3H), 7.37 (d, J = 8.0 Hz, 1H), 7.09 − 7.04 (m, 1H), 6.24 − 6.22 (m, 2H), 6.20 (dd, J = 3.5, 1.9 Hz, 1H), 3.39 (td, J = 7.0, 2.4 Hz, 2H), 2.77 (td, J = 7.0, 1.4 Hz, 2H), 2.21 (s, 3H). | 292.1 |
| 177 | 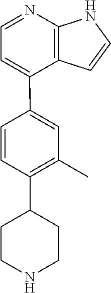 [SMILES: Cc1cc(-c2ccnc3[nH]ccc23)ccc1C1CCNCC1] | 4-(3-methyl-4- (piperidin-4- yl)phenyl)-1H- pyrrolo[2,3- b]pyridine | | 292.1 |
| 178 | 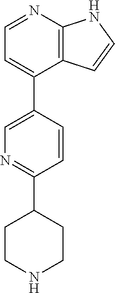 [SMILES: c1cc(-c2ccc(C3CCNCC3)nc2)c2cc[nH]c2n1] | 4-(6-(piperidin- 4-yl)pyridin-3- yl)-1H- pyrrolo[2,3- b]pyridine | | 279.1 |
| 179 | 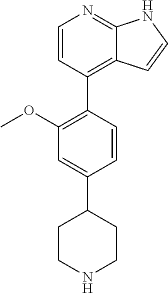 [SMILES: COc1cc(C2CCNCC2)ccc1-c1ccnc2[nH]ccc12] | 4-(2-methoxy-4- (piperidin-4- yl)phenyl)-1H- pyrrolo[2,3- b]pyridine | ¹H NMR (400 MHz, DMSO-d₆) δ 11.75 (s, 1H), 8.62 (bs, 1H), 8.37 (bs, 1H), 8.24 (d, J = 4.9 Hz, 1H), 7.47 (dd, J = 3.5, 2.5 Hz, 1H), 7.39 (d, J = 7.8 Hz, 1H), 7.10 (t, J = 5.0 Hz, 1H), 7.03 (d, J = 1.6 Hz, 1H), 6.96 (dd, J = 7.8, 1.6 Hz, 1H), 6.26 (dd, J = 3.5, 1.8 Hz, 1H), 3.77 (s, 3H), 3.42 (d, J = 13.0 Hz, 2H), 3.15 − 2.83 (m, 3H), 2.15 − 1.97 (m, 2H), 1.87 (qd, J = 13.2, 4.0 Hz, 2H). | 308.2 |
| 180 | 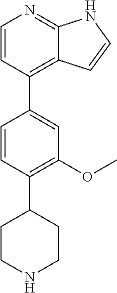 [SMILES: COc1cc(-c2ccnc3[nH]ccc23)ccc1C1CCNCC1] | 4-(3-methoxy-4- (piperidin-4- yl)phenyl)-1H- pyrrolo[2,3- b]pyridine | | 308.1 |
| 181 | 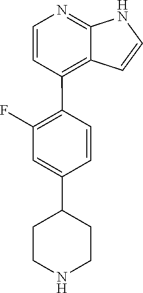 [SMILES: Fc1cc(C2CCNCC2)ccc1-c1ccnc2[nH]ccc12] | 4-(2-fluoro-4- (piperidin-4- yl)phenyl)-1H- pyrrolo[2,3- b]pyridine | ¹H NMR (400 MHz, DMSO-d₆) δ 11.80 (s, 1H), 8.56 (bs, 1H), 8.32 (bs, 1H), 8.27 (d, J = 4.9 Hz, 1H), 7.61 (t, J = 8.1 Hz, 1H), 7.52 (dd, J = 3.5, 2.5 Hz, 1H), 7.29 − 7.20 (m, 2H), 7.10 (dd, J = 4.9, 1.4 Hz, 1H), 6.33 (dt, J = 3.8, 2.0 Hz, 1H), 3.40 (d, J = 12.5 Hz, 2H), 3.11 − 2.89 (m, 3H), 2.03 (d, J = 13.2 Hz, 2H), 1.81 (qd, J = 13.2, 3.9 Hz, 2H). | 296.1 |
| 182 | 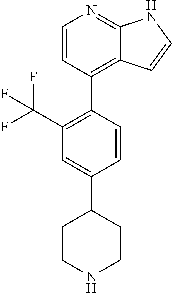 [SMILES: FC(F)(F)c1cc(C2CCNCC2)ccc1-c1ccnc2[nH]ccc12] | 4-(4-(piperidin- 4-yl)-2- (trifluoromethyl) phenyl)-1H- pyrrolo[2,3- b]pyridine | | 346.1 |
| 183 | 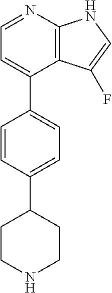 [SMILES: Fc1c[nH]c2nccc(-c3ccc(C4CCNCC4)cc3)c12] | 3-fluoro-4-(4- (piperidin-4- yl)phenyl)-1H- pyrrolo[2,3- b]pyridine | ¹H NMR (400 MHz, DMSO-d₆) δ 11.65 (s, 1H), 8.57 (d, J = 13.5 Hz, 1H), 8.31 (d, J = 4.9 Hz, 1H), 7.64 (dd, J = 8.2, 2.9 Hz, 2H), 7.52 (t, J = 2.4 Hz, 1H), 7.45 − 7.34 (m, 2H), 7.14 (d, J = 4.9 Hz, 1H), 3.41 (d, J = 12.5 Hz, 2H), 3.13 − 2.85 (m, 3H), 2.02 (d, J = 13.8 Hz, 2H), 1.96 − 1.72 (m, 2H). | 296.1 |
| 184 | 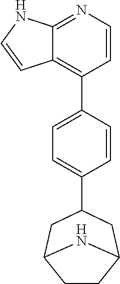 [SMILES: c1cc(-c2ccc(C3CC4CCC(C3)N4)cc2)c2cc[nH]c2n1] | 4-(4-(8- azabicyclo[3.2.1] octan-3- yl)phenyl)-1H- pyrrolo[2,3- b]pyridine | | 304.1 |
| 185 | 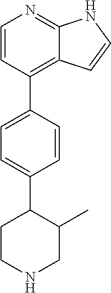 [SMILES: CC1CNCCC1c1ccc(-c2ccnc3[nH]ccc23)cc1] | 4-(4-(3- methylpiperidin- 4-yl)phenyl)- 1H-pyrrolo[2,3- b]pyridine | ¹H NMR (400 MHz, DMSO-d₆) δ 11.86 (s, 1H), 8.29 (d, J = 5.0 Hz, 1H), 8.12 (bs, 1H), 7.82 − 7.74 (m, 2H), 7.56 (dd, J = 3.5, 2.5 Hz, 1H), 7.43 − 7.36 (m, 2H), 7.22 (d, J = 5.1 Hz, 1H), 6.64 (dd, J = 3.5, 1.9 Hz, 1H), 3.40 (d, J = 12.4 Hz, 1H), 3.27 (s, 2H), 3.22 (dt, J = 12.7, 3.9 Hz, 1H), 3.04 (q, J = 13.4, 12.7 Hz, 1H), 2.40 − 2.28 (m, 1H), 2.17 (qd, J = 12.9, 4.0 Hz, 1H), 1.87 (d, J = 14.3 Hz, 1H), 0.80 (d, J = 7.4 Hz, 3H). | 292.1 |
| 186 | 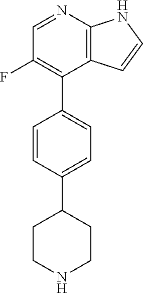 [SMILES: Fc1cnc2[nH]ccc2c1-c1ccc(C2CCNCC2)cc1] | 5-fluoro-4-(4- (piperidin-4- yl)phenyl)-1H- pyrrolo[2,3- b]pyridine | ¹H NMR (400 MHz, DMSO-d₆) δ 11.93 (s, 1H), 8.43 (s, 2H), 8.28 (d, J = 3.0 Hz, 1H), 7.76 − 7.51 (m, 3H), 7.51 − 7.31 (m, 2H), 6.43 (dd, J = 3.5, 1.9 Hz, 1H), 3.40 (d, J = 12.8 Hz, 2H), 3.03 (td, J = 12.7, 2.9 Hz, 2H), 2.94 (ddd, J = 12.1, 8.6, 3.5 Hz, 1H), 2.01 (d, J = 13.8 Hz, 2H), 1.83 (qd, J = 13.3, 4.0 Hz, 2H). | 296.1 |
| 187 | 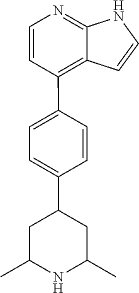 [SMILES: CC1CC(c2ccc(-c3ccnc4[nH]ccc34)cc2)CC(C)N1] | 4-(4-(2,6- dimethylpiperidin- 4-yl)phenyl)- 1H-pyrrolo[2,3- b]pyridine | ¹H NMR (400 MHz, DMSO-d₆) δ 11.87 (s, 1H), 8.60 (d, J = 69.2 Hz, 1H), 8.28 (d, J = 5.0 Hz, 1H), 7.89 − 7.71 (m, 2H), 7.50 − 7.32 (m, 2H), 7.19 (d, J = 5.0 Hz, 1H), 6.62 (dd, J = 3.5, 1.9 Hz, 1H), 3.11 (dt, J = 67.9, 11.8 Hz, 1H), 2.11 − 1.77 (m, 2H), 1.57 (q, J = 12.4 Hz, 1H), 1.43 (d, J = 7.1 Hz, 1H), 1.27 (dd, J = 8.9, 6.4 Hz, 5H). | 306.2 |
| 188 | 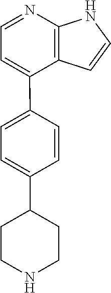 [SMILES: c1cc(-c2ccc(C3CCNCC3)cc2)c2cc[nH]c2n1] | 4-(4-(piperidin- 4-yl)phenyl)- 1H-pyrrolo[2,3- b]pyridine | ¹H NMR (400 MHz, Methanol-d₄) δ 8.44 (d, J = 6.2 Hz, 1H), 7.99 − 7.79 (m, 2H), 7.74 (d, J = 3.6 Hz, 1H), 7.65 (d, J = 6.2 Hz, 1H), 7.62 − 7.50 (m, 2H), 6.99 (d, J = 3.6 Hz, 1H), 3.63 − 3.41 (m, 2H), 3.19 (td, J = 12.9, 3.0 Hz, 2H), 3.06 (tt, J = 12.1, 3.7 Hz, 1H), 2.23 − 2.11 (m, 2H), 2.11 − 1.88 (m, 2H). | 278.1 |

### Compound 189

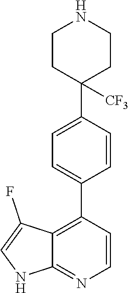 [SMILES: Fc1c[nH]c2nccc(-c3ccc(C4(C(F)(F)F)CCNCC4)cc3)c12]

### 3-fluoro-4-(4-(4-(trifluoromethyl)piperidin-4-yl)phenyl)-1H-pyrrolo[2,3-b]pyri dine

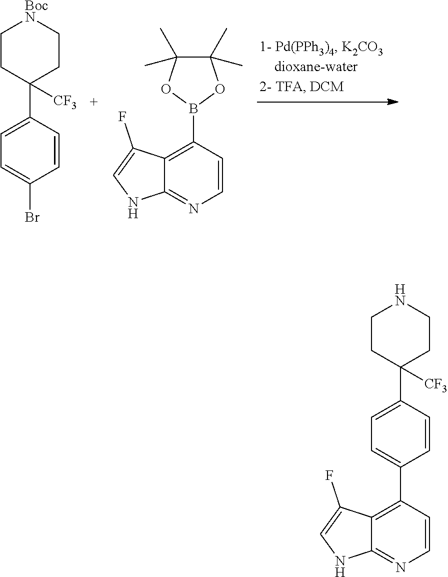 [SMILES: CC(C)(C)OC(=O)N1CCC(c2ccc(Br)cc2)(C(F)(F)F)CC1 ; CC1(C)OB(c2ccnc3[nH]cc(F)c23)OC1(C)C ; Fc1c[nH]c2nccc(-c3ccc(C4(C(F)(F)F)CCNCC4)cc3)c12]

A microwave vial was charged with tert-butyl 4-(4-bromophenyl)-4-(trifluoromethyl)piperidine-1-carboxylate (100 mg, 0.245 mmol), 3-fluoro-4-(4,4,5,5-tetramethyl-1,3,2-dioxaborolan-2-yl)-1H-pyrrolo[2,3-b]pyridine (70 mg, 0.269 mmol) and XPhosPd(crotyl) Cl (8.3 mg, 0.012 mmol). The vial was capped, was then purged with nitrogen. Then, tetrahydrofuran (740 μL) was added, followed by 2 M aqueous potassium phosphate tribasic (184 μL, 0.367 mmol). The content of the reaction vessel was purged again, then the reaction mixture was stirred at 90° C. for 30 min. The reaction mixture was partitioned between 10 mL of water and 10 mL of tert-butyl methyl ether. The layers were separated; the aqueous layer was extracted with two 10 mL portions of tert-butyl methyl ether. The combined organic layer was washed with 10 mL of brine, was then dried (sodium sulfate), and was then concentrated under diminished pressure. The residue was applied to a silica gel column (Isco, 12 g); eluting with 100:0→0:100 hexanes-ethyl acetate afforded the title compound; ¹H NMR (400 MHz, Chloroform-d) δ 10.46 (s, 1H), 8.39 (d, J=5.0 Hz, 1H), 7.75 (dd, J=8.5, 2.8 Hz, 2H), 7.57 (d, J=8.3 Hz, 2H), 7.20 (d, J=2.0 Hz, 1H), 7.18 (d, J=5.0 Hz, 1H), 4.07 (s, 2H), 2.83 (s, 2H), 2.52 (d, J=13.9 Hz, 2H), 2.10 (td, J=13.4, 4.3 Hz, 2H), 1.45 (s, 9H).

A solution containing tert-butyl 4-(4-(3-fluoro-1H-pyrrolo[2,3-b]pyridin-4-yl)phenyl)-4-(trifluoromethyl)piperidine-1-carboxylate (0.060 g, 0.129 mmol) in dioxane (0.650 mL) was treated with 4 M hydrochloric acid solution in dioxane (0.324 mL 1.295 mmol) at 23° C. for 18 hr. The reaction suspension was concentrated under diminished pressure. The residue was dissolved into 1.5 mL of MeOH and the resulting solution was purified by HPLC to the title compound; ¹H NMR (400 MHz, DMSO-d₆) δ 11.69 (s, 1H), 8.34 (d, J=4.9 Hz, 1H), 8.20 (s, 1H), 7.76 (dd, J=8.6, 2.7 Hz, 2H), 7.71 (d, J=8.5 Hz, 2H), 7.55 (t, J=2.3 Hz, 1H), 7.22 (d, J=4.9 Hz, 1H), 3.03 (d, J=12.8 Hz, 2H), 2.61 (d, J=13.6 Hz, 2H), 2.03 (td, J=13.3, 3.5 Hz, 2H).

### Compound 190

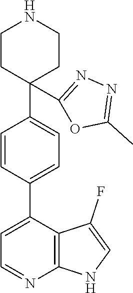 [SMILES: Cc1nnc(C2(c3ccc(-c4ccnc5[nH]cc(F)c45)cc3)CCNCC2)o1]

### 2-(4-(4-(3-fluoro-1H-pyrrolo[2,3-b]pyridin-4-yl)phenyl)piperidin-4-yl)-5-methyl-1,3,4-oxadiazole

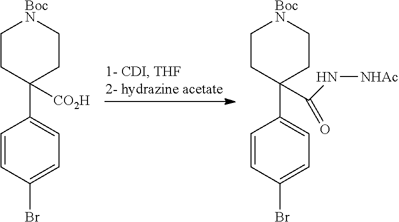 [SMILES: CC(C)(C)OC(=O)N1CCC(C(=O)O)(c2ccc(Br)cc2)CC1 ; CC(=O)NNC(=O)C1(c2ccc(Br)cc2)CCN(C(=O)OC(C)(C)C)CC1]

### tert-butyl 4-(2-acetylhydrazine-1-carbonyl)-4-(4-bromophenyl)piperidine-1-carboxylate

A solution containing 4-(4-bromophenyl)-1-(tert-butoxycarbonyl)piperidine-4-carboxylic acid (0.500 g, 1.30 mmol) in tetrahydrofuran (2.60 mL) was treated with acetylhydrazide (0.289 g, 3.90 mmol) at 23° C. for 2.5 hr. Then, acetylhydrazide (0.289 g, 3.90 mmol) was added, and the reaction mixture was stirred at 23° C. for 24 hr. The reaction mixture was concentrated under diminished pressure. The residue was applied to a silica gel column; eluting with 100:0→90:10 dichloromethane-methanol (methanol containing 1% aq. ammonium hydroxide) afforded the title compound; ¹H NMR (400 MHz, DMSO-d₆) δ 9.63 (s, 1H), 9.57 (s, 1H), 7.55-7.50 (m, 2H), 7.38-7.31 (m, 2H), 3.86-3.75 (m, 2H), 3.14-2.97 (m, 2H), 2.49-2.44 (m, 2H), 1.83 (s, 3H), 1.66 (td, J=13.1, 12.6, 4.2 Hz, 2H), 1.39 (s, 9H).

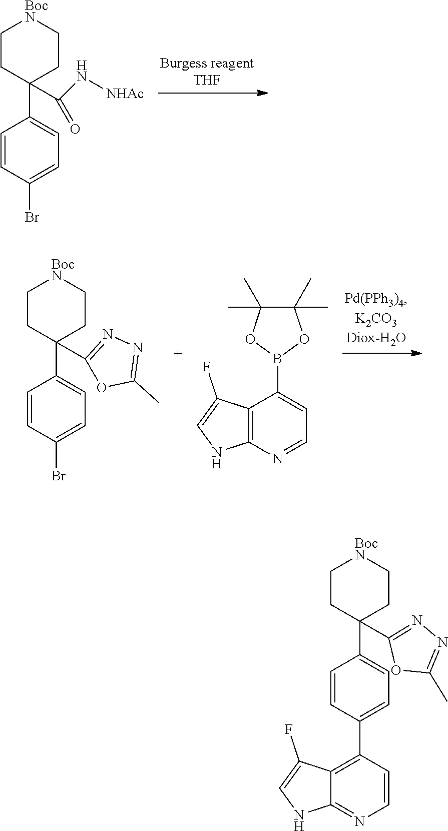

### tert-butyl 4-(4-(3-fluoro-1H-pyrrolo[2,3-b]pyridin-4-yl)phenyl)-4-(5-methyl-1,3,4-oxadiazol-2-yl)piperidine-1-carboxylate

A solution containing tert-butyl 4-(2-acetylhydrazine-1-carbonyl)-4-(4-bromophenyl)piperidine-1-carboxylate (0.150 g, 0.341 mmol) and Burgess reagent (0.097 g, 0.409 mmol) in tetrahydrofuran (3.40 mL) was heated in the microwave reactor at 80° C. for 30 min. The reaction mixture was allowed to cool down to room temperature, and was then concentrated under diminished pressure. The residue was transferred to a microwave vial. Then, tert-butyl 4-(4-bromophenyl)-4-(5-methyl-1,3,4-oxadiazol-2-yl)piperidine-1-carboxylate (0.083 g, 0.197 mmol), 3-fluoro-4-(4,4,5,5-tetramethyl-1,3,2-dioxaborolan-2-yl)-1H-pyrrolo[2,3-b]pyridine (0.057 g, 0.216 mmol) and XPhos Pd(crotyl)Cl (6.62 mg, 9.83 μmol) were added. The vial was capped and the content of the reaction vessel was then flushed with nitrogen. Then dioxane (0.590 mL) and 2 M aqueous potassium phosphate tribasic (0.147 mL, 0.295 mmol) were added. The content of the reaction vessel was then flushed with nitrogen. The resulting mixture was heated in the microwave reactor at 120° C. for 45 min. The organic layer was pipetted out of the reaction vessel. The reaction mixture was washed with 1 mL of TBME. The collected organic solution was then concentrated under diminished pressure. The residue was applied to a silica gel column; eluting with 100:0→90:10 dichloromethane-methanol (methanol containing 10% aq. ammonium hydroxide) afforded the title compound; ¹H NMR (400 MHz, DMSO-d₆) δ 11.66 (s, 1H), 8.31 (d, J=4.9 Hz, 1H), 7.66 (dd, J=8.5, 2.9 Hz, 2H), 7.53 (t, J=2.4 Hz, 1H), 7.50-7.44 (m, 2H), 7.14 (d, J=4.9 Hz, 1H), 3.88 (d, J=13.5 Hz, 2H), 2.61-2.55 (m, 3H), 2.46 (s, 3H), 2.18-2.09 (m, 2H), 1.41 (s, 9H).

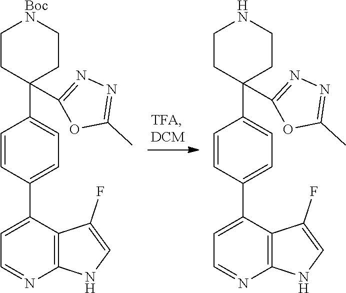 [SMILES: Cc1nnc(C2(c3ccc(-c4ccnc5[nH]cc(F)c45)cc3)CCN(C(=O)OC(C)(C)C)CC2)o1 ; Cc1nnc(C2(c3ccc(-c4ccnc5[nH]cc(F)c45)cc3)CCNCC2)o1]

### 2-(4-(4-(3-fluoro-1H-pyrrolo[2,3-b]pyridin-4-yl)phenyl)piperidin-4-yl)-5-methyl-1,3,4-oxadiazole

A sample of tert-butyl 4-(4-(3-fluoro-1H-pyrrolo[2,3-b]pyridin-4-yl)phenyl)-4-(5-methyl-1,3,4-oxadiazol-2-yl)piperidine-1-carboxylate (40 mg, 0.084 mmol) was stirred in 4:1 dichloromethane-trifluoroacetic acid (500 μL) at 23° C. for 3 hr. The reaction mixture was concentrated under diminished pressure. The residue was dissolved into 1 mL of methanol, and the resulting solution was purified by HPLC to afford the title compound; ¹H NMR (400 MHz, DMSO-d₆) δ 11.66 (s, 1H), 8.31 (d, J=5.0 Hz, 1H), 7.66 (d, J=8.5 Hz, 2H), 7.53 (d, J=2.9 Hz, 1H), 7.44 (d, J=8.5 Hz, 2H), 7.14 (d, J=4.9 Hz, 1H), 3.03 (m, 2H), 2.74-2.65 (m, 2H), 2.64-2.58 (m, 2H), 2.46 (s, 3H), 2.28-2.16 (m, 2H).

### Compound 191

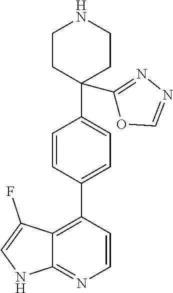 [SMILES: Fc1c[nH]c2nccc(-c3ccc(C4(c5nnco5)CCNCC4)cc3)c12]

### 2-(4-(4-(3-fluoro-1H-pyrrolo[2,3-b]pyridin-4-yl)phenyl)piperidin-4-yl)-1,3,4-oxadiazole

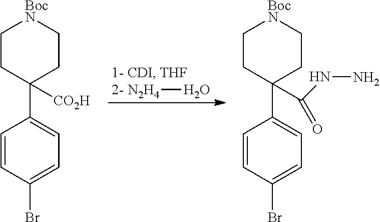

### tert-butyl 4-(4-bromophenyl)-4-(hydrazinecarbonyl)piperidine-1-carboxylate

A solution containing 4-(4-bromophenyl)-1-(tert-butoxycarbonyl)piperidine-4-carboxylic acid (0.500 g, 1.30 mmol) in tetrahydrofuran (2.60 mL) was treated with N,N-carbonyldiimidazole (0.232 g, 1.43 mmol) at 23° C. for 3 hr. Then, hydrazine monohydrate (0.190 mL, 3.90 mmol) was added, and the reaction solution was further stirred at 23° C. for 2 hr. The reaction mixture was concentrated under diminished pressure. The residue was applied to a silica gel column; eluting with 100:0→90:10 dichloromethane-methanol (MeOH containing 1% aq. ammonium hydroxide) afforded the title compound; ¹H NMR (400 MHz, Chloroform-d) δ 7.55-7.47 (m, 2H), 7.25-7.19 (m, 2H), 6.46 (s, 1H), 3.78 (d, J=3.7 Hz, 2H), 3.59-3.38 (m, 4H), 2.39-2.28 (m, 2H), 2.09-1.93 (m, 2H), 1.58 (s, 2H), 1.44 (d, J=0.5 Hz, 9H).

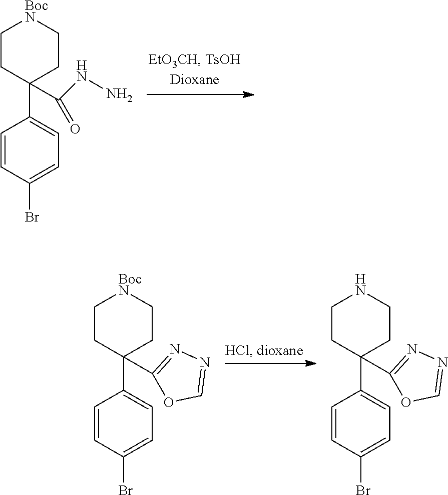 [SMILES: CC(C)(C)OC(=O)N1CCC(C(=O)NN)(c2ccc(Br)cc2)CC1 ; CC(C)(C)OC(=O)N1CCC(c2ccc(Br)cc2)(c2nnco2)CC1 ; Brc1ccc(C2(c3nnco3)CCNCC2)cc1]

### 2-(4-(4-bromophenyl)piperidin-4-yl)-1,3,4-oxadiazole

A solution containing tert-butyl 4-(4-bromophenyl)-4-(hydrazinecarbonyl)piperidine-1-carboxylate (0.410 g, 1.03 mmol) and tosic acid (0.020 g, 0.103 mmol) in triethyl orthoformate (1.70 mL, 10.3 mmol) was heated in the microwave reactor at 140° C. for 1 hr. The reaction mixture was partitioned between 15 mL of saturated aqueous sodium bicarbonate and 15 mL of ethyl acetate. The layers were separated; the aqueous layer was extracted with two 15 mL portions of ethyl acetate. The combined organic layer was washed with 15 mL of brine, was then dried (sodium sulfate), and was then concentrated under diminished pressure. The obtained residue was dissolved in dioxane (2.40 mL), and the resulting solution was treated with 4 M hydrochloric acid solution in dioxane (2.40 mL, 9.60 mmol) at 23° C. for 3.5 hr. The reaction mixture was concentrated under diminished pressure. The residue was applied to a C-18 column; eluting with 100:0-0:100 water-acetonitrile (+0.1% formic acid) afforded the tilted compound; ¹H NMR (400 MHz, DMSO-d₆) δ 9.19 (s, 1H), 8.26 (s, 1H), 7.59-7.55 (m, 2H), 7.29-7.23 (m, 2H), 3.05 (dt, J=13.1, 4.0 Hz, 2H), 2.79-2.69 (m, 2H), 2.56 (d, J=13.6 Hz, 2H), 2.21 (ddd, J=14.4, 11.1, 3.9 Hz, 2H).

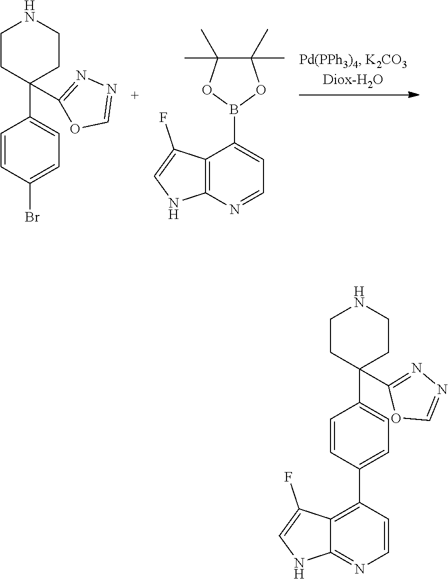 [SMILES: Brc1ccc(C2(c3nnco3)CCNCC2)cc1 ; CC1(C)OB(c2ccnc3[nH]cc(F)c23)OC1(C)C ; Fc1c[nH]c2nccc(-c3ccc(C4(c5nnco5)CCNCC4)cc3)c12]

### 2-(4-(4-(3-fluoro-1H-pyrrolo[2,3-b]pyridin-4-yl)phenyl)piperidin-4-yl)-1,3,4-oxadiazole

A microwave vial was charged with 2-(4-(4-bromophenyl)piperidin-4-yl)-1,3,4-oxadiazole (0.100 g, 0.324 mmol), 3-fluoro-4-(4,4,5,5-tetramethyl-1,3,2-dioxaborolan-2-yl)-1H-pyrrolo[2,3-b]pyridine (0.094 g, 0.357 mmol) and XPhos Pd(crotyl)Cl (10.93=mg, 0.016 mmol). The content of the reaction vessel was then flushed with nitrogen. Then dioxane (0.970 mL) and 2 M aqueous potassium phosphate tribasic (0.243 ml, 0.487 mmol) were added. The content of the reaction vessel was then flushed with nitrogen. The resulting mixture was heated in the microwave reactor at 120° C. for 45 min. The reaction mixture was diluted with 10 mL of MeOH. The resulting suspension was filtered through a pad of celite. The collected filtrate was then concentrated under diminished pressure. The residue was purified by HPLC to afford the titled compound; ¹H NMR (400 MHz, DMSO-d₆) δ 11.66 (s, 1H), 9.21 (s, 1H), 8.30 (d, J=4.9 Hz, 1H), 8.26 (s, 1H), 7.66 (dd, J=8.6, 2.9 Hz, 2H), 7.53 (t, J=2.3 Hz, 1H), 7.47-7.42 (m, 2H), 7.14 (d, J=4.9 Hz, 1H), 3.03 (d, J=13.0 Hz, 2H), 2.74-2.65 (m, 2H), 2.64-2.58 (m, 2H), 2.28-2.16 (m, 2H).

### Compound 192

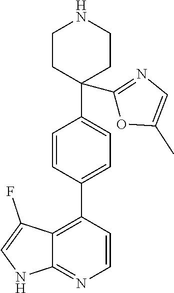 [SMILES: Cc1cnc(C2(c3ccc(-c4ccnc5[nH]cc(F)c45)cc3)CCNCC2)o1]

### 2-(4-(4-(3-fluoro-1H-pyrrolo[2,3-b]pyridin-4-yl)phenyl)piperidin-4-yl)-5-methyloxazole

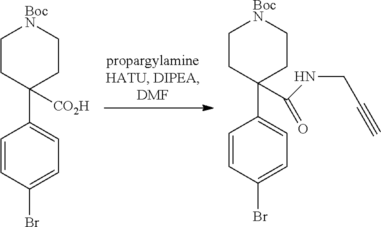 [SMILES: CC(C)(C)OC(=O)N1CCC(C(=O)O)(c2ccc(Br)cc2)CC1 ; C#CCNC(=O)C1(c2ccc(Br)cc2)CCN(C(=O)OC(C)(C)C)CC1]

### tert-butyl 4-(4-bromophenyl)-4-(prop-2-yn-1-ylcarbamoyl)piperidine-1-carboxylate

A solution containing 4-(4-bromophenyl)-1-(tert-butoxycarbonyl)piperidine-4-carboxylic acid (0.500 g, 1.30 mmol) and propargylamine (0.092 ml, 1.43 mmol) in N,N-dimethylformamide (3.25 mL) was treated with HATU (0.544 g, 1.431 mmol) and diisopropethylamine (0.340 mL, 1.95 mmol) at 23° C. for 3 hr. The reaction mixture was partitioned between 15 mL of saturated aqueous sodium bicarbonate and 15 mL of ethyl acetate. The layers were separated; the aqueous layer was extracted with three 15 mL portions of ethyl acetate. The combined organic layer was washed with 15 mL of water, then with 15 mL of brine, was then dried (sodium sulfate), and was then concentrated under diminished pressure. The residue was applied to a silica gel column; eluting with 100:0→0:100 Hex-EtOAc afforded the title compound; ¹H NMR (400 MHz, Chloroform-d) δ 7.54-7.47 (m, 2H), 7.25-7.20 (m, 2H), 5.34 (t, J=5.1 Hz, 1H), 3.95 (dd, J=5.3, 2.6 Hz, 2H), 3.64 (s, 1H), 3.37 (s, 2H), 2.31 (d, J=13.9 Hz, 2H), 2.17 (t, J=2.5 Hz, 1H), 1.56 (s, 2H), 1.44 (s, 9H).

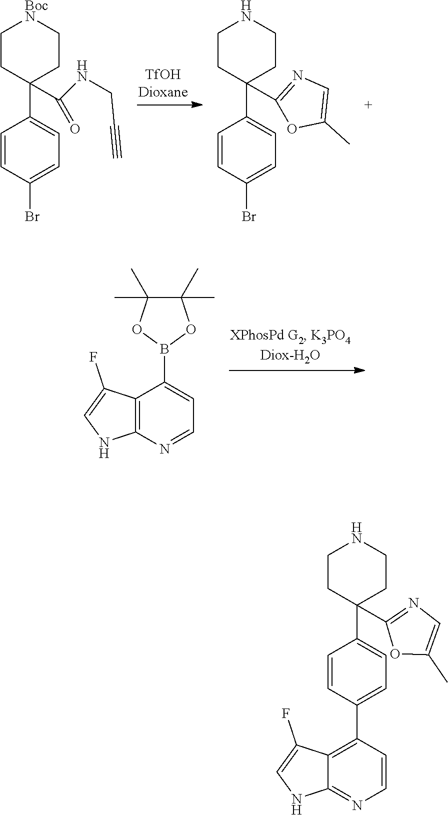 [SMILES: C#CCNC(=O)C1(c2ccc(Br)cc2)CCN(C(=O)OC(C)(C)C)CC1 ; Cc1cnc(C2(c3ccc(Br)cc3)CCNCC2)o1 ; CC1(C)OB(c2ccnc3[nH]cc(F)c23)OC1(C)C ; Cc1cnc(C2(c3ccc(-c4ccnc5[nH]cc(F)c45)cc3)CCNCC2)o1]

### 2-(4-(4-(3-fluoro-1H-pyrrolo[2,3-b]pyridin-4-yl)phenyl)piperidin-4-yl)-5-methyloxazole

A solution containing tert-butyl 4-(4-bromophenyl)-4-(prop-2-yn-1-ylcarbamoyl)piperidine-1-carboxylate (0.160 g, 0.380 mmol) in dioxane (1.15 mL) was carefully treated with triflic acid (0.067 mL, 0.759 mmol). The reaction mixture was heated in the microwave reactor at 140° C. for 30 min. The reaction mixture was allowed to cool down to room temperature, and was then partitioned between 10 mL of saturated aqueous sodium bicarbonate and 10 mL of ethyl acetate. The layers were separated; the aqueous layer was extracted with two 10 mL portions of ethyl acetate. The combined organic layer was washed with 10 mL of brine, was then dried (sodium sulfate), and was then concentrated under diminished pressure. The residue was transferred to a microwave vial, followed by the addition of 3-fluoro-4-(4,4,5,5-tetramethyl-1,3,2-dioxaborolan-2-yl)-1H-pyrrolo[2,3-b]pyridine (80 mg, 0.305 mmol) and of XPhosPdG2 (6.60 mg, 0.014 mmol). The vial was capped, was then purged with nitrogen. Then, dioxane (840 μL) was added, followed by 2 M aqueous potassium phosphate tribasic (208 μL, 0.416 mmol). The content of the reaction vessel was purged again, then the reaction mixture was heated in the microwave reactor at 90° C. for 1 hr. The reaction mixture was diluted with 2 mL of methanol; the resulting solution was filtered through a thiol cartridge; the collected filtrate was concentrated under diminished pressure; the residue was purified by HPLC to afford the title compound; ¹H NMR (400 MHz, DMSO-d₆) δ 11.67 (s, 1H), 8.31 (d, J=4.9 Hz, 1H), 7.68 (dd, J=8.5, 2.9 Hz, 2H), 7.53 (dd, J=2.9, 2.0 Hz, 1H), 7.41 (d, J=8.5 Hz, 2H), 7.14 (d, J=4.9 Hz, 1H), 6.90 (q, J=1.1 Hz, 1H), 3.37 (d, J=18.6 Hz, 3H), 2.94 (d, J=10.8 Hz, 2H), 2.75 (d, J=14.1 Hz, 2H), 2.43-2.31 (m, 2H), 2.27 (d, J=1.3 Hz, 3H).

### Compound 193

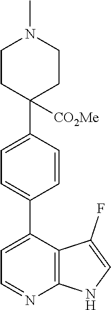 [SMILES: COC(=O)C1(c2ccc(-c3ccnc4[nH]cc(F)c34)cc2)CCN(C)CC1]

### Methyl 4-(4-(3-fluoro-1H-pyrrolo[2,3-b]pyridin-4-yl)phenyl)-1-methylpiperidine-4-carboxylate

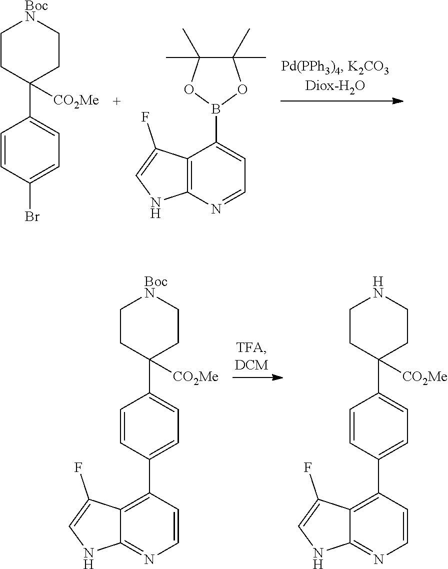 [SMILES: COC(=O)C1(c2ccc(Br)cc2)CCN(C(=O)OC(C)(C)C)CC1 ; CC1(C)OB(c2ccnc3[nH]cc(F)c23)OC1(C)C ; COC(=O)C1(c2ccc(-c3ccnc4[nH]cc(F)c34)cc2)CCN(C(=O)OC(C)(C)C)CC1 ; COC(=O)C1(c2ccc(-c3ccnc4[nH]cc(F)c34)cc2)CCNCC1]

### Methyl 4-(4-(3-fluoro-1H-pyrrolo[2,3-b]pyridin-4-yl)phenyl)piperidine-4-carboxylate

A microwave vial was charged with 1-(tert-butyl) 4-methyl 4-(4-bromophenyl)piperidine-1,4-dicarboxylate (0.200 g, 0.502 mmol), 3-fluoro-4-(4,4,5,5-tetramethyl-1,3,2-dioxaborolan-2-yl)-1H-pyrrolo[2,3-b]pyridine (0.145 g, 0.552 mmol) and palladium tetrakistriphenylphosphine (0.029 g, 0.025 mmol). The vial was capped, and was then purged with nitrogen twice. Then, dioxane (2.50 mL) was added, followed by 2 M aqueous potassium carbonate (0.630 mL, 1.30 mmol). The content of the vial was purged with nitrogen twice, and was then heated in the microwave reactor at 150° C. for 30 min. The reaction mixture was partitioned between 10 mL of water and 10 mL of tert-butyl methyl ether. The layers were separated; the aqueous layer was extracted with three 10 mL portions of tert-butyl methyl ether. The combined organic layer was washed with 10 mL of brine, was then dried (sodium sulfate), and was then concentrated under diminished pressure. The residue was applied to a silica gel column; eluting with 100:0→80:20 dichloromethane-methanol afforded 1-(tert-butyl) 4-methyl 4-(4-(3-fluoro-1H-pyrrolo[2,3-b]pyridin-4-yl)phenyl)piperidine-1,4-dicarboxylate. A solution containing 1-(tert-butyl) 4-methyl 4-(4-(3-fluoro-1H-pyrrolo[2,3-b]pyridin-4-yl)phenyl)piperidine-1,4-dicarboxylate (0.200 g, 0.441 mmol) in dichloromethane (2.04 mL) was treated with trifluoroacetic acid (0.510 ml, 6.62 mmol) at 23° C. for 4 hr. The reaction mixture was concentrated under diminished pressure The residue was dissolved into 10 mL of 90:10 dichloromethane-methanol. The solution was treated with two 10-mL portions of saturated aqueous NaHCO₃. The combined aqueous layer was extracted with three 15 mL portions of 9:1 dichloromethane-methanol. The combined organic layer was washed with 10 mL of brine, was then dried (Na₂SO₄), and was then concentrated under diminished pressure to afford the title compound.

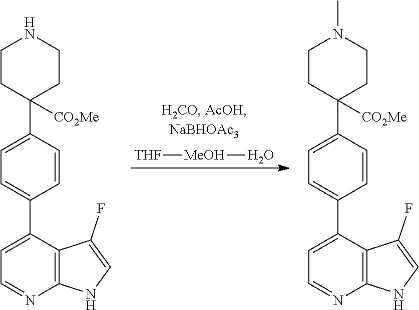

### Methyl 4-(4-(3-fluoro-1H-pyrrolo[2,3-b]pyridin-4-yl)phenyl)-1-methylpiperidine-4-carboxylate

A solution containing methyl 4-(4-(3-fluoro-1H-pyrrolo[2,3-b]pyridin-4-yl)phenyl)piperidine-4-carboxylate (100 mg, 0.283 mmol) in 1:1 methanol-tetrahydrofuran (2.80 mL) was treated with formaldehyde (37% aqueous) (63 μL, 0.849 mmol), sodium triacetoxyborohydride (72 mg, 0.340 mmol) and acetic acid (10 μL, 0.175 mmol) at 23° C. for 2.5 hr. The reaction mixture was concentrated under diminished pressure. The residue was purified by HPLC to afford the title compound; ¹H NMR (400 MHz, Chloroform-d) δ 8.29 (dd, J=9.8, 5.0 Hz, 1H), 7.68-7.61 (m, 1H), 7.57 (dd, J=8.4, 2.8 Hz, 1H), 7.51-7.44 (m, 1H), 7.41 (d, J=8.4 Hz, 1H), 7.19 (dd, J=21.7, 2.3 Hz, 1H), 7.10 (ddd, J=13.1, 5.0, 0.5 Hz, 1H), 5.79 (s, 1H), 3.69 (d, J=6.5, 0.5 Hz, 3H), 2.92 (d, J=11.5 Hz, 3H), 2.60 (dd, J=24.9, 13.2 Hz, 2H), 2.32 (d, J=2.0 Hz, 3H), 2.28-2.18 (m, 1H), 2.15-2.05 (m, 1H), 2.01 (s, 1H).

### Compounds 194-201

Compounds 194-201 were all prepared using the synthetic procedures described above, substituting the appropriate reactants and reagents that were prepared as described herein or are commercially available, and are shown in Table 26.

TABLE 26

| | | | | LC/MS |
|---|---|---|---|---|
| | | | | m/z |
| Compound | Structure | IUPAC Name | ¹H NMR | [M + H]⁺ |
| 194 | 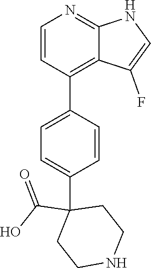 [SMILES: O=C(O)C1(c2ccc(-c3ccnc4[nH]cc(F)c34)cc2)CCNCC1] | 4-(4-(3-fluoro-1H- pyrrolo[2,3- b]pyridin-4- yl)phenyl)piperidine- 4-carboxylic acid | | 340.14 |
| 195 | 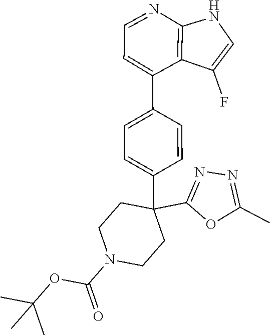 [SMILES: Cc1nnc(C2(c3ccc(-c4ccnc5[nH]cc(F)c45)cc3)CCN(C(=O)OC(C)(C)C)CC2)o1] | tert-butyl 4-(4-(3- fluoro-1H- pyrrolo[2,3- b]pyridin-4- yl)phenyl)-4-(5- methyl-1,3,4- oxadiazol-2- yl)piperidine-1- carboxylate | ¹H NMR (400 MHz, DMSO-d₆) δ 11.66 (s, 1H), 8.31 (d, J = 4.9 Hz, 1H), 7.66 (dd, J = 8.5, 2.9 Hz, 2H), 7.53 (t, J = 2.4 Hz, 1H), 7.51-7.41 (m, 2H), 7.14 (d, J = 4.9 Hz, 1H), 3.88 (d, J = 13.5 Hz, 2H), 3.06-2.92 (m, 2H), 2.65-2.53 (m, 3H), 2.46 (s, 3H), 2.20-2.00 (m, 2H), 1.41 (s, 9H). | 478.22 |
| 196 | 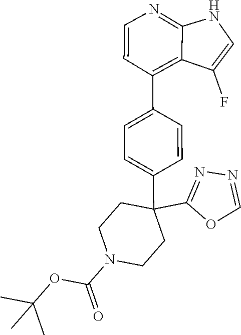 [SMILES: CC(C)(C)OC(=O)N1CCC(c2ccc(-c3ccnc4[nH]cc(F)c34)cc2)(c2nnco2)CC1] | tert-butyl 4-(4-(3- fluoro-1H- pyrrolo[2,3- b]pyridin-4- yl)phenyl)-4-(1,3,4- oxadiazol-2- yl)piperidine-1- carboxylate | | 464.2 |
| 197 | 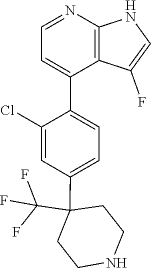 [SMILES: Fc1c[nH]c2nccc(-c3ccc(C4(C(F)(F)F)CCNCC4)cc3Cl)c12] | 4-(2-chloro-4-(4- (trifluoromethyl) piperidin-4-yl)phenyl)- 3-fluoro-1H- pyrrolo[2,3- b]pyridine | | 398.1 |
| 198 | 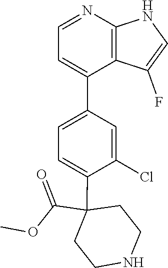 [SMILES: COC(=O)C1(c2ccc(-c3ccnc4[nH]cc(F)c34)cc2Cl)CCNCC1] | methyl 4-(2-chloro-4- (3-fluoro-1H- pyrrolo[2,3- b]pyridin-4- yl)phenyl)piperidine- 4-carboxylate | ¹H NMR (400 MHz, DMSO-d₆) δ 11.76 (s, 1H), 8.62 (bs, 1H), 8.43 (bs, 1H), 8.36 (d, J = 4.9 Hz, 1H), 7.78 (dt, J = 2.6, 1.1 Hz, 1H), 7.74 (d, J = 1.3 Hz, 2H), 7.59 (dd, J = 2.9, 2.0 Hz, 1H), 7.25 (d, J = 4.9 Hz, 1H), 3.67 (s, 3H), 3.33- 3.25 (s, 4H), 2.61- 2.53 (m, 2H), 2.36- 2.18 (m, 2H). | 388.11 |
| 199 | 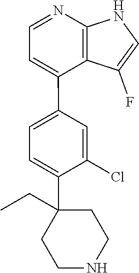 [SMILES: CCC1(c2ccc(-c3ccnc4[nH]cc(F)c34)cc2Cl)CCNCC1] | 4-(3-chloro-4-(4- ethylpiperidin-4- yl)phenyl)-3-fluoro- 1H-pyrrolo[2,3- b]pyridine | ¹H NMR (400 MHz, DMSO-d₆) δ 11.71 (s, 1H), 8.31 (d, J = 4.9 Hz, 1H), 7.70 (dd, J = 2.9, 1.9 Hz, 1H), 7.64 (dt, J = 8.1, 2.3 Hz, 1H), 7.55 (dd, J = 2.8, 2.0 Hz, 1H), 7.51 (d, J = 8.1 Hz, 1H), 7.18 (d, J = 4.9 Hz, 1H), 3.32 (d, J = 12.8 Hz, 1H), 3.20 (d, J = 12.4 Hz, 1H), 3.13 (dd, J = 14.4, 6.7 Hz, 1H), 2.90-2.72 (m, 2H), 1.99 (d, J = 14.1 Hz, 1H), 1.92-1.80 (m, | 358.14 |
| | | | 1H), 1.53-1.28 (m, | |
| | | | 3H), 1.24 (d, J = 7.0 | |
| | | | Hz, 3H). | |
| 200 | 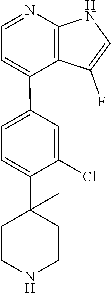 [SMILES: CC1(c2ccc(-c3ccnc4[nH]cc(F)c34)cc2Cl)CCNCC1] | 4-(3-chloro-4-(4- methylpiperidin-4- yl)phenyl)-3-fluoro- 1H-pyrrolo[2,3- b]pyridine | ¹H NMR (400 MHz, DMSO-d₆) δ 11.74 (d, J = 2.9 Hz, 1H), 8.47 (bs, 1H), 8.34 (d, J = 4.9 Hz, 1H), 8.30 (bs, 1H), 7.63 (dt, J = 3.6, 1.6 Hz, 1H), 7.60- 7.52 (m, 3H), 7.23 (d, J = 4.9 Hz, 1H), 3.25- 3.11 (m, 2H), 3.02- 2.91 (m, 2H), 2.33- 2.23 (m, 2H), 1.91 (ddd, J = 14.5, 8.1, 3.4 Hz, 2H), 1.29 (s, 3H). | 344.13 |
| 201 | 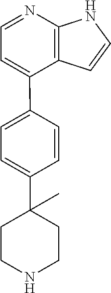 [SMILES: CC1(c2ccc(-c3ccnc4[nH]ccc34)cc2)CCNCC1] | 4-(4-(4- methylpiperidin-4- yl)phenyl)-1H- pyrrolo[2,3- b]pyridine | ¹H NMR (400 MHz, DMSO-d₆) δ 11.75 (s, 1H), 8.26 (d, J = 4.9 Hz, 1H), 7.82-7.66 (m, 2H), 7.61-7.39 (m, 3H), 7.18 (d, J = 4.9 Hz, 1H), 6.62 (dd, J = 3.3, 1.6 Hz, 1H), 2.94-2.84 (m, 2H), 2.79-2.71 (m, 2H), 2.08-1.99 (m, 2H), 1.75-1.68 (m, 2H), 1.25 (s, 3H). | 292.17 |

### Synthesis of Compounds 202-204

Compounds 202-204 can be prepared as shown below.

### Compound 202

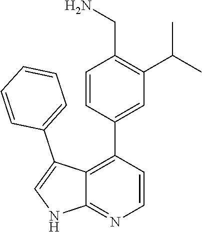 [SMILES: CC(C)c1cc(-c2ccnc3[nH]cc(-c4ccccc4)c23)ccc1CN]

### (2-isopropyl-4-(3-phenyl-1H-pyrrolo[2,3-b]pyridin-4-yl)phenyl)methanamine

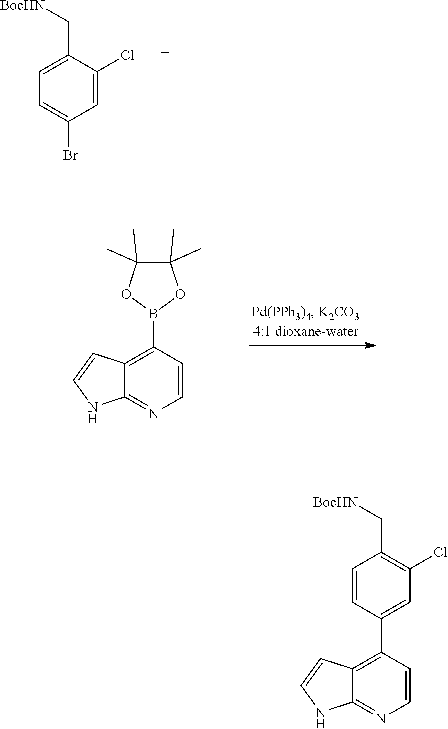 [SMILES: CC(C)(C)OC(=O)NCc1ccc(Br)cc1Cl ; CC1(C)OB(c2ccnc3[nH]ccc23)OC1(C)C ; CC(C)(C)OC(=O)NCc1ccc(-c2ccnc3[nH]ccc23)cc1Cl]

### Step R. tert-butyl (2-chloro-4-(1H-pyrrolo[2,3-b]pyridin-4-yl)benzyl)carbamate

A screw-capped vial was charged with 4-(4,4,5,5-tetramethyl-1,3,2-dioxaborolan-2-yl)-1H-pyrrolo[2,3-b]pyridine (1.09 g, 4.46 mmol), 4-(4,4,5,5-tetramethyl-1,3,2-dioxaborolan-2-yl)-1H-pyrrolo[2,3-b]pyridine (1.09 g, 4.46 mmol) and tetrakis(triphenylphosphine)palladium (0.234 g, 0.203 mmol). The vial was capped and its content was purged with nitrogen. Then, dioxane (8.20 mL) was added, followed by 2 M aqueous potassium carbonate (4.05 mL, 8.11 mmol). The content of the vial was purged with nitrogen and was then stirred at 120° C. for 20 hr. The reaction was allowed to cool down to room temperature; the formation of a white precipitate was observed. The solid was collected by filtration through a Buchner funnel to afford the title compound; ¹H NMR (400 MHz, DMSO-d₆) δ 8.29 (d, J=4.9 Hz, 1H), 7.79-7.73 (m, 2H), 7.57 (d, J=3.5 Hz, 1H), 7.48 (d, J=7.7 Hz, 1H), 7.43-7.37 (m, 1H), 7.21 (d, J=5.0 Hz, 1H), 6.59 (d, J=3.5 Hz, 1H), 4.28 (d, J=6.0 Hz, 2H), 1.42 (s, 9H).

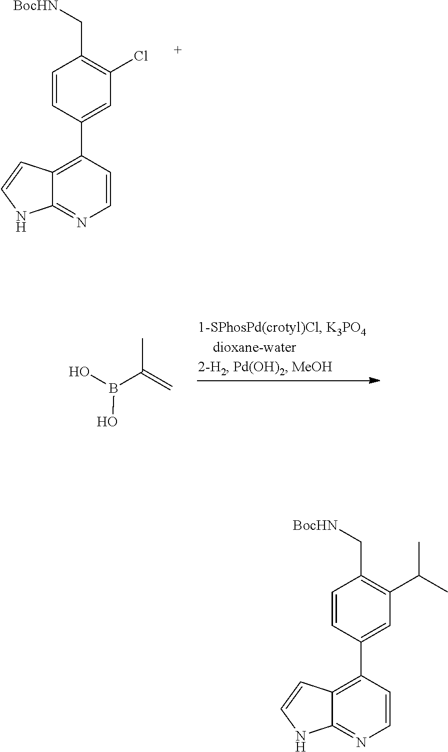

### Step S. tert-butyl (2-isopropyl-4-(1H-pyrrolo[2,3-b]pyridin-4-yl)benzyl)carbamate

A microwave vial was charged with tert-butyl (2-chloro-4-(1H-pyrrolo[2,3-b]pyridin-4-yl)benzyl)carbamate (0.450 g, 1.258 mmol), prop-1-en-2-ylboronic acid (0.162 g, 1.886 mmol) and SPhosPd(crotyl)Cl (0.076 g, 0.126 mmol). The vial was capped, and its content was purged with nitrogen twice. Then, dioxane (7.60 mL) was added, followed by 2 M aqueous potassium phosphate tribasic (1.886 ml, 3.77 mmol). The content of the reaction vessel was purged with nitrogen, then the reaction mixture was stirred at 150° C. for 30 min. The reaction mixture was diluted with 20 mL of tert-butyl methyl ether. The resulting suspension was filtered through a pad of celite to remove any black insoluble deposit, was washed with 10 mL of brine, was then dried (sodium sulfate), and was then concentrated under diminished pressure. The residue was applied to a silica gel column; eluting with 100:0→0:100 dichloromethane-ethyl acetate afforded the title compound; ¹H NMR (400 MHz, DMSO-d₆) δ 11.78 (s, 1H), 8.27 (d, J=4.9 Hz, 1H), 7.67 (dd, J=8.0, 2.0 Hz, 1H), 7.54 (dd, J=3.5, 2.5 Hz, 1H), 7.47 (d, J=1.9 Hz, 1H), 7.45 (d, J=8.2 Hz, 1H), 7.40 (t, J=6.1 Hz, 1H), 7.18 (d, J=4.9 Hz, 1H), 6.58 (dd, J=3.5, 1.9 Hz, 1H), 5.29 (p, J=1.6 Hz, 1H), 4.93 (dd, J=2.3, 1.2 Hz, 1H), 4.23 (d, J=6.1 Hz, 2H), 2.09 (t, J=1.2 Hz, 3H), 1.41 (s, 9H).

A solution containing tert-butyl (2-(prop-1-en-2-yl)-4-(1H-pyrrolo[2,3-b]pyridin-4-yl)benzyl)carbamate (0.355 g, 0.977 mmol) in methanol (65.0 mL) was passed through the H-cube, using the Pearlman's catalyst (0.137 g, 0.977 mmol) cartridge at a flow of 1 mL/min, at 80° C. under an atmosphere of hydrogen (40 bar). The collected solution was concentrated under diminished pressure to afford the title compound; ¹H NMR (400 MHz, Chloroform-d) δ 9.10 (s, 1H), 8.37 (d, J=4.9 Hz, 1H), 7.70 (d, J=1.8 Hz, 1H), 7.59-7.52 (m, 2H), 7.40 (d, J=7.9 Hz, 1H), 7.37 (dd, J=3.6, 2.0 Hz, 1H), 7.18 (d, J=4.8 Hz, 1H), 6.68 (dd, J=3.7, 1.6 Hz, 1H), 4.47 (d, J=5.7 Hz, 2H), 3.28 (p, J=6.9 Hz, 1H), 1.49 (s, 9H), 1.32 (d, J=6.8 Hz, 6H).

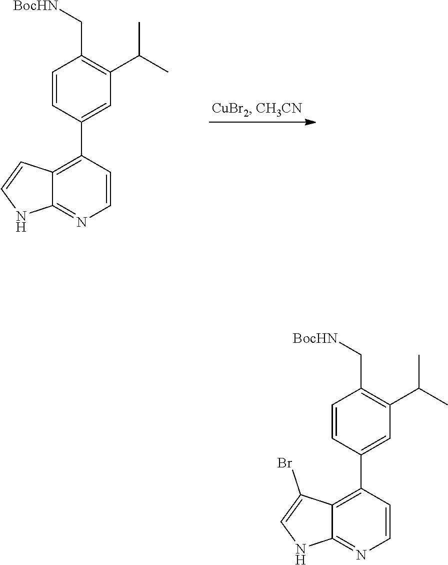 [SMILES: CC(C)c1cc(-c2ccnc3[nH]ccc23)ccc1CNC(=O)OC(C)(C)C ; CC(C)c1cc(-c2ccnc3[nH]cc(Br)c23)ccc1CNC(=O)OC(C)(C)C]

### Step T. tert-butyl (4-(3-bromo-1H-pyrrolo[2,3-b]pyridin-4-yl)-2-isopropylbenzyl)carbamate

A suspension containing tert-butyl (2-isopropyl-4-(1H-pyrrolo[2,3-b]pyridin-4-yl)benzyl)carbamate (0.200 g, 0.547 mmol) in acetonitrile (5.47 mL) was treated with copper(II) bromide (0.367 g, 1.64 mmol) at 23° C. for 2.5 hr. The resulting dark green suspension was treated with 7 M methanolic ammonia (2.35 mL, 16.42 mmol) and was stirred vigorously for 5 min. The mixture was filtered through a funnel equipped with a filter paper. The collected filtrate was partitioned between 15 mL of water and 15 mL of tert-butylmethyl ether. The layers were separated. The product was extracted with two 15-mL portions of tert-butylmethyl ether. The combined organic layer was washed with 15 mL of brine, was then dried (sodium sulfate), and was then concentrated under diminished pressure. The residue was applied to a silica gel column; eluting with 100:0→0:100 dichloromethane-ethyl acetate afforded the title compound; ¹H NMR (400 MHz, Chloroform-d) δ 7.46 (s, 1H), 7.38 (s, 2H), 7.36-7.30 (m, 3H), 4.47 (d, J=5.8 Hz, 3H), 3.33-3.17 (m, 1H), 2.75-2.61 (m, 1H), 1.49 (d, J=1.0 Hz, 9H), 1.29 (d, J=6.8 Hz, 6H).

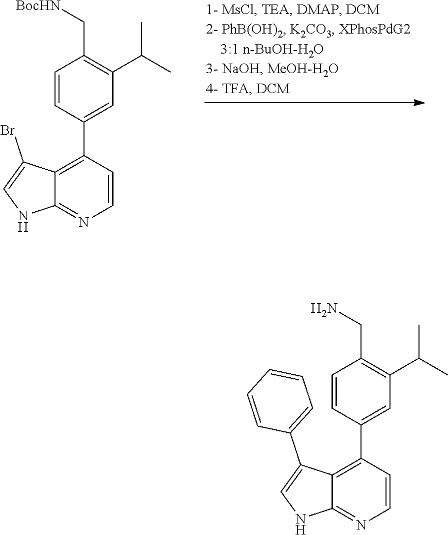 [SMILES: CC(C)c1cc(-c2ccnc3[nH]cc(Br)c23)ccc1CNC(=O)OC(C)(C)C ; CC(C)c1cc(-c2ccnc3[nH]cc(-c4ccccc4)c23)ccc1CN]

### Step U. (2-isopropyl-4-(3-phenyl-1H-pyrrolo[2,3-b]pyridin-4-yl)phenyl)methanamine

A solution containing tert-butyl (4-(3-bromo-1H-pyrrolo[2,3-b]pyridin-4-yl)-2-isopropylbenzyl)carbamate (0.140 g, 0.315 mmol) in DCM (3.15 mL) was treated with methanesulfonyl chloride (0.029 mL, 0.378 mmol), triethylamine (0.088 mL, 0.630 mmol) and DMAP (0.004 g, 0.032 mmol) at 23° C. for 5 hr. The reaction mixture was partitioned between 5 mL of saturated aqueous sodium bicarbonate and 5 mL of dichloromethane. The layers were separated (emulsion formed at the interface between the two layers); the aqueous layer was extracted with two 10 mL portions of dichloromethane. The combined organic layer was washed with 10 mL of brine, was then dried (sodium sulfate), and was then concentrated under diminished pressure. The residue was applied to a silica gel column; eluting with 100:0→0:100 hexanes-ethyl acetate afforded the title compound; ¹H NMR (400 MHz, Chloroform-d) δ 8.50 (dd, J=4.9, 0.4 Hz, 1H), 7.72 (d, J=0.4 Hz, 1H), 7.38 (d, J=1.8 Hz, 1H), 7.35 (d, J=7.9 Hz, 1H), 7.20 (d, J=5.0 Hz, 1H), 4.76 (s, 1H), 4.50-4.45 (m, 2H), 3.64 (s, 3H), 3.25 (p, J=6.8 Hz, 1H), 1.61 (s, 1H), 1.48 (s, 9H), 1.28 (d, J=6.8 Hz, 6H).

A microwave vial was charged with tert-butyl (4-(3-bromo-1-(methylsulfonyl)-1H-pyrrolo[2,3-b]pyridin-4-yl)-2-isopropylbenzyl)carbamate (54.0 mg, 0.103 mmol), phenylboronic acid (18.9 mg, 0.155 mmol), and XPhosPdG2 (4.1 mg, 5.17 μmol). The vial was capped, and its content was purged with nitrogen twice. Then, butan-1-ol (465 μL) was added, followed by 2 M aqueous potassium carbonate (155 μl, 0.310 mmol). The content of the reaction vial was purged with nitrogen and was then heated in the microwave reactor at 120° C. for 30 min. The organic layer was pipetted out, was filtered through a syringe equipped with a nylon filter, and the collected filtrate was concentrated using the blow-down unit. The obtained residue was dissolved in methanol (1.03 mL), and the resulting solution was treated with 4 M aqueous sodium hydroxide (0.260 ml, 1.040 mmol) in the microwave reactor at 100° C. for 30 min. The reaction mixture was diluted with 1 mL of MeOH, and the resulting suspension was filtered through a syringe equipped with a nylon filter. The collected filtrate was concentrated using the blow-down unit. The residue was dissolved into 1.00 mL DCM and was treated with trifluoroacetic acid (0.135 mL, 1.755 mmol) at 23° C. for 72 hr. The resulting solution was diluted with approx. 0.50 mL of MeOH and was purified by HPLC purification to afford the title compound; ¹H NMR (400 MHz, DMSO-d₆) δ 12.11 (s, 1H), 8.33 (d, J=4.8 Hz, 1H), 8.10 (s, 2H), 7.66 (d, J=2.7 Hz, 1H), 7.38-7.35 (m, 2H), 7.05 (d, J=4.8 Hz, 1H), 7.03-6.96 (m, 3H), 6.94-6.87 (m, 3H), 4.05 (q, J=5.8 Hz, 2H), 2.91 (p, J=6.6 Hz, 1H), 0.75 (d, J=6.8 Hz, 6H).

### Compound 203

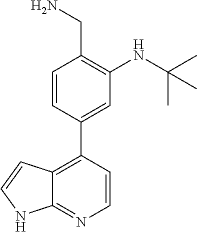 [SMILES: CC(C)(C)Nc1cc(-c2ccnc3[nH]ccc23)ccc1CN]

### 2-(aminomethyl)-N-(tert-butyl)-5-(1H-pyrrolo[2,3-b]pyridin-4-yl)aniline

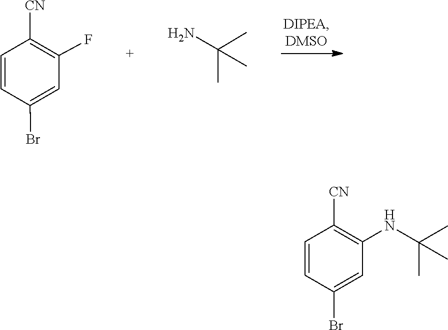

### Step V. 4-bromo-2-(tert-butylamino)benzonitrile

A solution containing 4-bromo-2-fluorobenzonitrile (0.200 g, 1.00 mmol), 2-methylpropan-2-amine (0.200 g, 2.73 mmol) and N,N-diisopropylethylamine (0.270 mL, 1.55 mmol) in dimethylsulfoxide (2.00 mL) was heated in the microwave reactor at 150° C. for 20 min. The reaction mixture was allowed to cool down to room temperature, and was then partitioned between 15 mL of water and 15 mL of diethyl ether. The layers were separated. The product was extracted with two 15-mL portions of diethyl ether. The combined organic layer was washed with brine (15 mL), was then dried (sodium sulfate), and was then concentrated under diminished pressure. The obtained residue was applied to a silica gel column; eluting with 100:0→0:100 hexanes-ethyl acetate afforded the title compound.

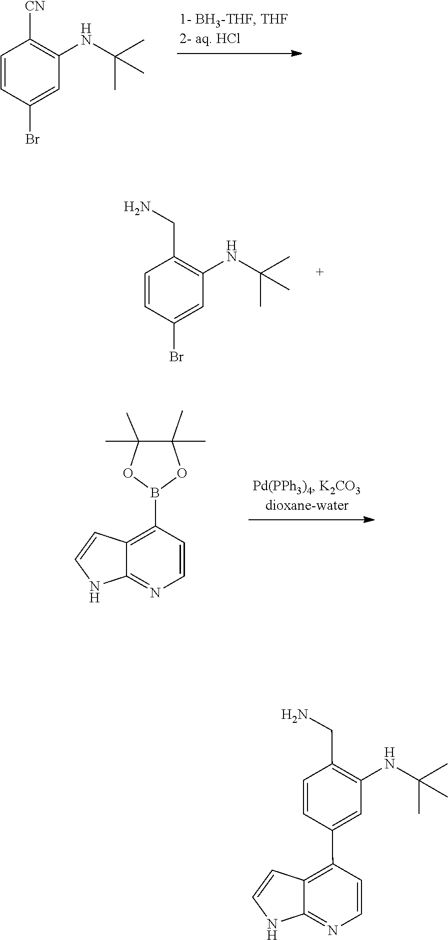 [SMILES: CC(C)(C)Nc1cc(Br)ccc1C#N ; CC(C)(C)Nc1cc(Br)ccc1CN ; CC1(C)OB(c2ccnc3[nH]ccc23)OC1(C)C ; CC(C)(C)Nc1cc(-c2ccnc3[nH]ccc23)ccc1CN]

### Step W. 2-(aminomethyl)-N-(tert-butyl)-5-(1H-pyrrolo[2,3-b]pyridin-4-yl)aniline

A solution containing 4-bromo-2-(tert-butylamino)benzonitrile (0.185 g, 0.731 mmol) in tetrahydrofuran (1.50 mL) was treated with a 1 M borane solution in tetrahydrofuran (5.00 mL, 5 mmol) at 23° C. for 16 h. The reaction mixture was diluted with 20 mL of water, and was then treated with 5 mL of 1 M aqueous hydrochloric acid. Then, saturated aqueous sodium bicarbonate was added until an alkaline pH was reached. The product was extracted with three 15-mL portions of ethyl acetate. The combined organic layer was washed with brine (15 mL), was then dried (sodium sulfate), and was then concentrated under diminished pressure. A screw-capped vial was charged with the obtained crude benzylamine, 4-(4,4,5,5-tetramethyl-1,3,2-dioxaborolan-2-yl)-1H-pyrrolo[2,3-b]pyridine (0.06 g, 0.205 mmol) and tetrakis(triphenylphosphine)palladium (0.010 g, 0.002 mmol). The vial was capped and its content was purged with nitrogen. Then, dioxane (1.10 mL) was added, followed by 2 M aqueous potassium carbonate (0.360 mL, 0.720 mmol). The content of the vial was purged with nitrogen and was then heated in the microwave reactor at 150° C. for 30 min. The reaction mixture was allowed to cool down to room temperature, and was then partitioned between 5 mL of water and 5 mL of ethyl acetate. The layers were separated. The product was extracted with two 5-mL portions of ethyl acetate. The combined organic layer was washed with brine (5 mL), was then dried (sodium sulfate), and was then concentrated under diminished pressure. The obtained residue was purified by HPLC to afford the title compound.

### Compound 204

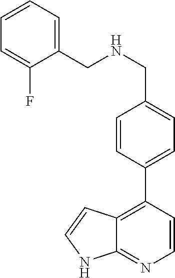 [SMILES: Fc1ccccc1CNCc1ccc(-c2ccnc3[nH]ccc23)cc1]

### N-(4-(1H-pyrrolo[2,3-b]pyridin-4-yl)benzyl)-1-(2-fluorophenyl)methanamine

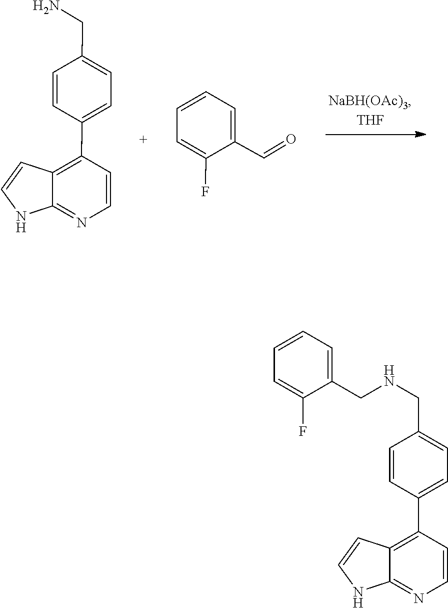 [SMILES: NCc1ccc(-c2ccnc3[nH]ccc23)cc1 ; O=Cc1ccccc1F ; Fc1ccccc1CNCc1ccc(-c2ccnc3[nH]ccc23)cc1]

Step X: A solution containing (4-(1H-pyrrolo[2,3-b]pyridin-4-yl)phenyl)methanamine (0.100 g, 0.448 mmol) and 2-fluorobenzaldehyde (0.056 g, 0.448 mmol) in tetrahydrofuran (4.50 mL) was treated with sodium triacetoxyborohydride (0.285 g, 1.34 mmol) at 23° C. for 3 h. The reaction mixture was treated with 5 mL of saturated aqueous sodium bicarbonate, and was then partitioned with 5 mL of ethyl acetate. The layers were separated. The product was extracted with two 5-mL portions of ethyl acetate. The combined organic layer was washed with brine (5 mL), was then dried (sodium sulfate), and was then concentrated under diminished pressure. The obtained residue was purified by HPLC to afford the title compound; ¹H NMR (400 MHz, DMSO-d₆) δ 11.90 (s, 1H), 9.37 (s, 2H), 8.31 (d, J=5.0 Hz, 1H), 7.91-7.84 (m, 2H), 7.69 (d, J=8.3 Hz, 2H), 7.65-7.57 (m, 2H), 7.53-7.46 (m, 1H), 7.38-7.28 (m, 2H), 7.23 (d, J=5.0 Hz, 1H), 6.61 (dd, J=3.5, 1.9 Hz, 1H), 4.32 (dt, J=21.0, 5.3 Hz, 4H).

### Compounds 205-287

Compounds 205-287 were all prepared using the synthetic procedures described above, substituting the appropriate reactants and reagents that were prepared as described herein or are commercially available, and are shown in Table 27.

TABLE 27

| | | | | LC/MS m/z |
|---|---|---|---|---|
| Compound | Structure | IUPAC Name | ¹H NMR | [M + H]⁺ |
| 205 | 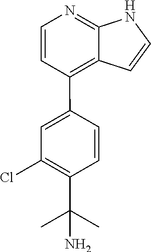 [SMILES: CC(C)(N)c1ccc(-c2ccnc3[nH]ccc23)cc1Cl] | 2-(2-chloro-4- (1H-pyrrolo[2,3- b]pyridin-4- yl)phenyl)propan- 2-amine | ¹H NMR (400 MHz, Methanol-d₄) δ 8.25 (d, J = 5.0 Hz, 1H), 7.86- 7.75 (m, 2H), 7.70 (dd, J = 8.2, 1.9 Hz, 1H), 7.47 (d, J = 3.6 Hz, 1H), 7.20 (d, J = 5.1 Hz, 1H), 6.65 (d, J = 3.5 Hz, 1H), 1.70 (s, 6H). | 285.9 |
| 206 | 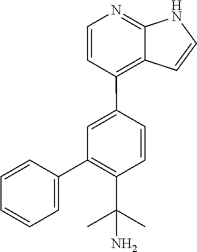 [SMILES: CC(C)(N)c1ccc(-c2ccnc3[nH]ccc23)cc1-c1ccccc1] | 2-(5-(1H- pyrrolo[2,3- b]pyridin-4-yl)- [1,1′-biphenyl]- 2-yl)propan-2- amine | ¹H NMR (400 MHz, Methanol-d₄) δ 8.22 (d, J = 5.1 Hz, 1H), 7.86- 7.71 (m, 2H), 7.49-7.36 (m, 7H), 7.20 (d, J = 5.1 Hz, 1H), 6.64 (d, J = 3.6 Hz, 1H), 1.47 (s, 6H). | 327.9 |
| 207 | 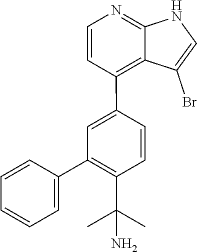 [SMILES: CC(C)(N)c1ccc(-c2ccnc3[nH]cc(Br)c23)cc1-c1ccccc1] | 2-(5-(3-bromo- 1H-pyrrolo[2,3- b]pyridin-4-yl)- [1,1′-biphenyl]- 2-yl)propan-2- amine | ¹H NMR (400 MHz, Methanol-d₄) δ 8.26 (d, J = 5.0 Hz, 1H), 7.74 (d, J = 8.3 Hz, 1H), 7.51 (dd, J = 8.3, 2.1 Hz, 1H), 7.48 (s, 1H), 7.46-7.37 (m, 5H), 7.17 (d, J = 2.0 Hz, 1H), 7.07 (d, J = 5.0 Hz, 1H), 1.47 (s, 6H). | 405.8 |
| 208 | 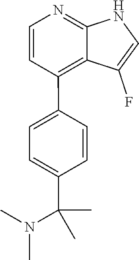 [SMILES: CN(C)C(C)(C)c1ccc(-c2ccnc3[nH]cc(F)c23)cc1] | 2-(4-(3-fluoro- 1H-pyrrolo[2,3- b]pyridin-4- yl)phenyl)-N,N- dimethylpropan- 2-amine | ¹H NMR (400 MHz, Methanol-d₄) δ 8.26 (d, J = 5.0 Hz, 1H), 7.66 (s, 4H), 7.26 (d, J = 2.2 Hz, 1H), 7.18 (d, J = 5.0 Hz, 1H), 2.21 (s, 6H), 1.49 (s, 6H). | 297.9 |
| 209 | 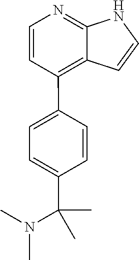 [SMILES: CN(C)C(C)(C)c1ccc(-c2ccnc3[nH]ccc23)cc1] | 2-(4-(1H- pyrrolo[2,3- b]pyridin-4- yl)phenyl)-N,N- dimethylpropan- 2-amine | | 279.9 |
| 210 | 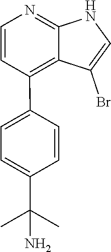 [SMILES: CC(C)(N)c1ccc(-c2ccnc3[nH]cc(Br)c23)cc1] | 2-(4-(3-bromo- 1H-pyrrolo[2,3- b]pyridin-4- yl)phenyl)propan- 2-amine | ¹H NMR (400 MHz, Methanol-d₄) δ 8.56 (bs, 1H), 7.85 (s, 1H), 7.74 (s, 4H), 7.49 (d, J = 5.5 Hz, 1H), 1.83 (s, 6H). | 329.8 |
| 211 | 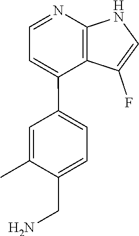 [SMILES: Cc1cc(-c2ccnc3[nH]cc(F)c23)ccc1CN] | (4-(3-fluoro-1H- pyrrolo[2,3- b]pyridin-4-yl)- 2- methylphenyl) methanamine | ¹H NMR (400 MHz, DMSO-d₆) δ 11.68 (s, 1H), 8.33 (d, J = 4.9 Hz, 1H), 8.16 (s, 3H), 7.61- 7.41 (m, 5H), 7.15 (d, J = 4.9 Hz, 1H), 4.12 (q, J = 5.8 Hz, 2H), 2.44 (s, 3H). | 256.2 |
| 212 | 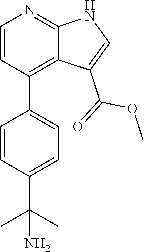 [SMILES: COC(=O)c1c[nH]c2nccc(-c3ccc(C(C)(C)N)cc3)c12] | methyl 4-(4-(2- aminopropan-2- yl)phenyl)-1H- pyrrolo[2,3- b]pyridine-3- carboxylate | ¹H NMR (400 MHz, DMSO-d₆) δ 12.64 (s, 1H), 8.45 (bs, 3H), 8.36 (d, J = 4.8 Hz, 1H), 8.23 (d, J = 3.0 Hz, 1H), 7.61- 7.54 (m, 2H), 7.48- 7.42 (m, 2H), 7.12 (d, J = 4.8 Hz, 1H), 3.20 (s, 3H), 1.68 (s, 6H). | 310.1 |
| 213 | 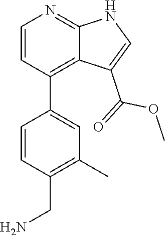 [SMILES: COC(=O)c1c[nH]c2nccc(-c3ccc(CN)c(C)c3)c12] | methyl 4-(4- (aminomethyl)- 3- methylphenyl)- 1H-pyrrolo[2,3- b]pyridine-3- carboxylate | ¹H NMR (400 MHz, DMSO-d₆) δ 12.62 (s, 1H), 8.36 (d, J = 4.9 Hz, 1H), 8.22 (d, J = 3.0 Hz, 1H), 8.16 (s, 3H), 7.42 (d, J = 8.3 Hz, 1H), 7.28 (d, J = 4.5 Hz, 2H), 7.10 (d, J = 4.9 Hz, 1H), 4.12 (d, J = 5.6 Hz, 2H), 3.28 (s, 3H), 2.40 (s, 3H). | 297.2 |
| 214 | 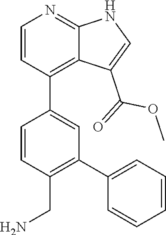 [SMILES: COC(=O)c1c[nH]c2nccc(-c3ccc(CN)c(-c4ccccc4)c3)c12] | methyl 4-(6- (aminomethyl)- [1,1′-biphenyl]- 3-yl)-1H- pyrrolo[2,3- b]pyridine-3- carboxylate | ¹H NMR (400 MHz, DMSO-d₆) δ 12.65 (s, 1H), 8.38 (d, J = 5.0 Hz, 1H), 8.24 (d, J = 2.9 Hz, 1H), 8.21 (s, 3H), 7.68 (d, J = 8.1 Hz, 1H), 7.60- 7.54 (m, 1H), 7.50 (d, J = 7.1 Hz, 2H), 7.43 (d, J = 7.5 Hz, 3H), 7.34- 6.95 (m, 2H), 4.06 (d, J = 5.7 Hz, 2H), 3.37 (s, 3H). | 358.2 |
| 215 | 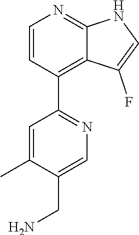 [SMILES: Cc1cc(-c2ccnc3[nH]cc(F)c23)ncc1CN] | (6-(3-fluoro-1H- pyrrolo[2,3- b]pyridin-4-yl)- 4-methylpyridin- 3- yl)methanamine | ¹H NMR (400 MHz, DMSO-d₆) δ 11.75 (s, 1H), 8.69 (s, 1H), 8.38 (d, J = 4.9 Hz, 1H), 8.20 (s, 3H), 7.74 (d, J = 3.2 Hz, 1H), 7.59 (t, J = 2.4 Hz, 1H), 7.41 (d, J = 4.9 Hz, 1H), 4.19 (d, J = 5.8 Hz, 2H), 2.48 (s, 3H). | 257.0 |
| 216 | 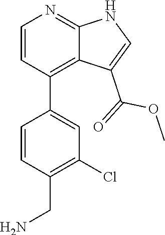 [SMILES: COC(=O)c1c[nH]c2nccc(-c3ccc(CN)c(Cl)c3)c12] | methyl 4-(4- (aminomethyl)- 3-chlorophenyl)- 1H-pyrrolo[2,3- b]pyridine-3- carboxylate | ¹H NMR (400 MHz, Methanol-d₄) δ 8.38- 8.33 (m, 2H), 8.19 (s, 1H), 7.59 (d, J = 7.9 Hz, 1H), 7.54 (d, J = 1.7 Hz, 1H), 7.43 (dd, J = 7.9, 1.7 Hz, 1H), 7.16 (d, J = 4.9 Hz, 1H), 4.36 (s, 2H), 3.50 (s, 3H). | 316.0 |
| 217 | 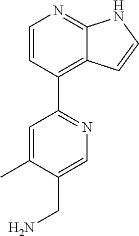 [SMILES: Cc1cc(-c2ccnc3[nH]ccc23)ncc1CN] | (4-methyl-6- (1H-pyrrolo[2,3- b]pyridin-4- yl)pyridin-3- yl)methanamine | ¹H NMR (400 MHz, DMSO-d₆) δ 11.85 (s, 1H), 8.72 (s, 1H), 8.34 (d, J = 5.1 Hz, 1H), 8.20 (s, 3H), 7.99 (s, 1H), 7.70 -7.39 (m, 2H), 6.99 (dd, J = 3.4, 1.8 Hz, 1H), 4.19 (d, J = 5.8 Hz, 2H), 2.51 (s, 3H). | 239.1 |
| 218 | 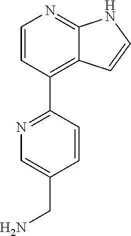 [SMILES: NCc1ccc(-c2ccnc3[nH]ccc23)nc1] | (6-(1H- pyrrolo[2,3- b]pyridin-4- yl)pyridin-3- yl)methanamine | ¹H NMR (400 MHz, DMSO-d₆) δ 11.86 (s, 1H), 8.85 (dd, J = 2.3, 0.8 Hz, 1H), 8.34 (d, J = 5.0 Hz, 1H), 8.22 (bs, 3 Hz, 3H), 8.17 (dd, J = 8.2, 0.9 Hz, 1H), 8.05 (dd, J = 8.2, 2.4 Hz, 1H), 7.65-7.59 (m, 2H), 6.98 (dd, J = 3.5, 1.9 Hz, 1H), 4.18 (q, J = 5.7 Hz, 2H). | 225.1 |
| 219 | 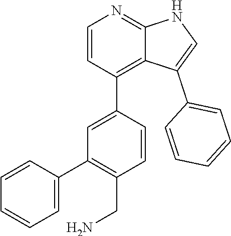 [SMILES: NCc1ccc(-c2ccnc3[nH]cc(-c4ccccc4)c23)cc1-c1ccccc1] | (5-(3-phenyl-1H- pyrrolo[2,3- b]pyridin-4-yl)- [1,1′-biphenyl]- 2- yl)methanamine | ¹H NMR (400 MHz, DMSO-d₆) δ 12.30 (s, 1H), 12.11 (d, J = 2.7 Hz, 2H), 8.35 (d, J = 4.9 Hz, 1H), 8.33 (d, J = 4.8 Hz, 1H), 8.21 (bs, 3H), 8.15 (bs, 5H), 7.76 (d, J = 2.8 Hz, 1H), 7.72 (d, J = 8.0 Hz, 1H), 7.68-7.64 (m, 3H), 7.57 (d, J = 1.5 Hz, 3H), 7.52-7.44 (m, 5H), 7.43 (d, J = 1.9 Hz, 1H), 7.34-7.28 (m, 4H), 7.14 (d, J = 4.9 Hz, 1H), 7.10- 7.02 (m, 6H), 6.94 (t, J = | 376.2 |
| | | | 1.6 Hz, 2H), 6.92 (q, J = | |
| | | | 1.6 Hz, 1H), 6.82 (t, J = | |
| | | | 1.2 Hz, 2H), 6.79 (ddd, | |
| | | | J = 4.9, 3.5, 1.7 Hz, 3H), | |
| | | | 4.05 (d, J = 5.9 Hz, 2H), | |
| | | | 3.90 (q, J = 5.8 Hz, 4H). | |
| 220 | 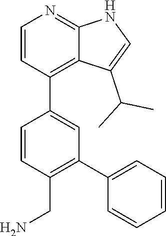 [SMILES: CC(C)c1c[nH]c2nccc(-c3ccc(CN)c(-c4ccccc4)c3)c12] | (5-(3-isopropyl- 1H-pyrrolo[2,3- b]pyridin-4-yl)- [1,1′-biphenyl]- 2- yl)methanamine | | 342.2 |
| 221 | 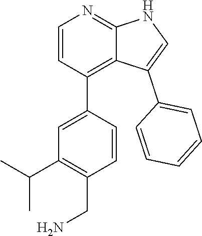 [SMILES: CC(C)c1cc(-c2ccnc3[nH]cc(-c4ccccc4)c23)ccc1CN] | (2-isopropyl-4- (3-phenyl-1H- pyrrolo[2,3- b]pyridin-4- yl)phenyl) methanamine | ¹H NMR (400 MHz, DMSO-d₆) δ 12.11 (s, 1H), 8.33 (d, J = 4.8 Hz, 1H), 8.10 (s, 3H), 7.66 (d, J = 2.7 Hz, 1H), 7.42- 7.31 (m, 2H), 7.04 (d, J = 4.8 Hz, 1H), 7.02- 6.96 (m, 3H), 6.93-6.86 (m, 3H), 4.05 (q, J = 5.7 Hz, 2H), 2.96-2.74 (m, 1H), 0.75 (d, J = 6.8 Hz, 6H). | 342.2 |
| 222 | 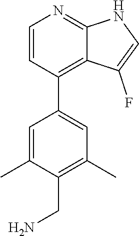 [SMILES: Cc1cc(-c2ccnc3[nH]cc(F)c23)cc(C)c1CN] | (4-(3-fluoro-1H- pyrrolo[2,3- b]pyridin-4-yl)- 2,6- dimethylphenyl) methanamine | ¹H NMR (400 MHz, DMSO-d₆) δ 11.60 (s, 1H), 8.31 (d, J = 4.9 Hz, 1H), 7.99 (s, 3H), 7.50 (t, J = 2.4 Hz, 1H), 7.40 (d, J = 2.9 Hz, 2H), 7.12 (d, J = 4.9 Hz, 1H), 4.13 (d, J = 5.9 Hz, 2H), 2.48 (s, 6H). | 270.1 |
| 223 | 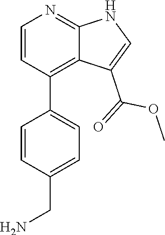 [SMILES: COC(=O)c1c[nH]c2nccc(-c3ccc(CN)cc3)c12] | methyl 4-(4- (aminomethyl) phenyl)-1H- pyrrolo[2,3- b]pyridine-3- carboxylate | ¹H NMR (400 MHz, DMSO-d₆) δ 12.62 (s, 1H), 8.35 (d, J = 4.9 Hz, 1H), 8.28-8.04 (m, 4H), 7.63 (d, J = 8.3 Hz, 3H), 7.49 (d, J = 8.0 Hz, 2H), 7.40 (t, J = 8.6 Hz, 4H), 7.08 (d, J = 4.9 Hz, 1H), 4.11 (s, 2H), 3.26 (s, 3H). | 282.1 |
| 224 | 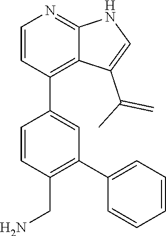 [SMILES: C=C(C)c1c[nH]c2nccc(-c3ccc(CN)c(-c4ccccc4)c3)c12] | (5-(3-(prop-1-en- 2-yl)-1H- pyrrolo[2,3- b]pyridin-4-yl)- [1,1′-biphenyl]- 2- yl)methanamine | ¹H NMR (400 MHz, DMSO-d₆) δ 11.89 (s, 1H), 8.29 (d, J = 4.9 Hz, 1H), 8.24-8.15 (m, 3H), 7.70 (d, J = 8.1 Hz, 1H), 7.64 (dd, J = 8.0, 1.9 Hz, 1H), 7.54-7.47 (m, 3H), 7.47-7.41 (m, 3H), 7.31 (d, J = 1.9 Hz, 1H), 7.11 (d, J = 4.9 Hz, 1H), 4.77 (dd, J = 2.4, 1.4 Hz, 1H), 4.51 (dd, J = 2.4, 1.0 Hz, 1H), 4.04 (p, J = 5.8 Hz, 2H), 1.60 (dd, J = 1.4, 0.8 Hz, 3H). | 340.1 |
| 225 | 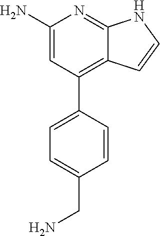 [SMILES: NCc1ccc(-c2cc(N)nc3[nH]ccc23)cc1] | 4-(4- (aminomethyl) phenyl)-1H- pyrrolo[2,3- b]pyridin-6- amine | ¹H NMR (400 MHz, DMSO-d₆) δ 11.49 (s, 1H), 8.21 (s, 3H), 7.76 (d, J = 8.0 Hz, 2H), 7.63 (d, J = 8.0 Hz, 2H), 7.12 (s, 1H), 6.51 (s, 1H), 6.38 (s, 1H), 4.14 (t, J = 5.8 Hz, 2H). | 239.1 |
| 226 | 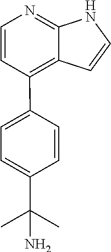 [SMILES: CC(C)(N)c1ccc(-c2ccnc3[nH]ccc23)cc1] | 2-(4-(1H- pyrrolo[2,3- b]pyridin-4- yl)phenyl)propan- 2-amine | ¹H NMR (400 MHz, DMSO-d₆) δ 11.79 (s, 1H), 8.42 (s, 3H), 8.30 (d, J = 5.0 Hz, 1H), 7.86 (d, J = 8.3 Hz, 2H), 7.71 (d, J = 8.3 Hz, 2H), 7.56 (t, J = 3.0 Hz, 1H), 7.21 (d, J = 5.0 Hz, 1H), 6.60 (dd, J = 3.5, 1.8 Hz, 1H), 1.70 (s, 6H). | 252.1 |
| 227 | 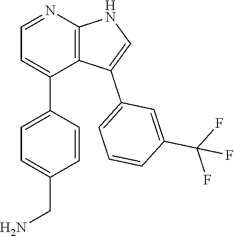 [SMILES: NCc1ccc(-c2ccnc3[nH]cc(-c4cccc(C(F)(F)F)c4)c23)cc1] | (4-(3-(3- (trifluoromethyl) phenyl)-1H- pyrrolo[2,3- b]pyridin-4- yl)phenyl) methanamine | ¹H NMR (400 MHz, DMSO-d₆) δ 12.17 (s, 1H), 8.35 (d, J = 4.8 Hz, 1H), 8.02 (d, J = 32.0 Hz, 4H), 7.78 (d, J = 2.6 Hz, 1H), 7.39-7.29 (m, 3H), 7.19 (q, J = 8.0 Hz, 4H), 7.07 (d, J = 4.8 Hz, 1H), 6.97 (s, 1H), 3.94 (d, J = 5.8 Hz, 2H). | 368.1 |
| 228 | 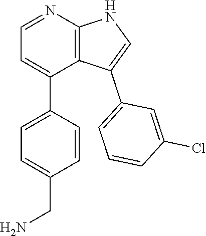 [SMILES: NCc1ccc(-c2ccnc3[nH]cc(-c4cccc(Cl)c4)c23)cc1] | (4-(3-(3- chlorophenyl)- 1H-pyrrolo[2,3- b]pyridin-4- yl)phenyl) methanamine | | 334.1 |
| 229 | 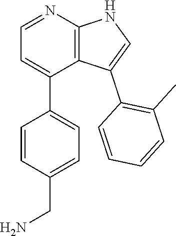 [SMILES: Cc1ccccc1-c1c[nH]c2nccc(-c3ccc(CN)cc3)c12] | (4-(3-(o-tolyl)- 1H-pyrrolo[2,3- b]pyridin-4- yl)phenyl) methanamine | ¹H NMR (400 MHz, Methanol-d₄) δ 8.26 (d, J = 5.0 Hz, 1H), 7.31 (s, 1H), 7.09-6.93 (m, 8H), 6.83 (d, J = 7.1 Hz, 1H), 3.66 (d, J = 1.4 Hz, 2H), 1.76 (s, 3H). | 314.1 |
| 230 | 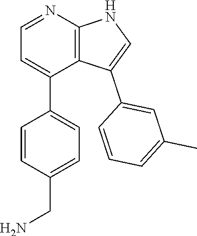 [SMILES: Cc1cccc(-c2c[nH]c3nccc(-c4ccc(CN)cc4)c23)c1] | (4-(3-(m-tolyl)- 1H-pyrrolo[2,3- b]pyridin-4- yl)phenyl) methanamine | ¹H NMR (400 MHz, Methanol-d₄) δ 13.41 (s, 1H), 9.36 (s, 1H), 9.16 (d, J = 4.9 Hz, 1H), 9.02 (d, J = 3.0 Hz, 1H), 8.51- 8.40 (m, 1H), 8.38- 8.31 (m, 2H), 8.25-8.15 (m, 2H), 7.93 (d, J = 4.9 Hz, 1H), 6.33 (s, 1H), 3.97 (s, 3H), 3.04-2.89 (m, 2H), | 314.1 |
| 231 | 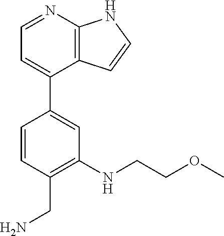 [SMILES: COCCNc1cc(-c2ccnc3[nH]ccc23)ccc1CN] | 2- (aminomethyl)- N-(2- methoxyethyl)- 5-(1H- pyrrolo[2,3- b]pyridin-4- yl)aniline | ¹H NMR (400 MHz, DMSO-d₆) δ 11.87 (s, 1H), 8.31 (d, J = 5.0 Hz, 1H), 8.19 (s, 3H), 7.68 (s, 1H), 7.58 (d, J = 4.2 Hz, 3H), 7.35-7.14 (m, 1H), 6.56 (s, 1H), 4.21 (d, J = 5.7 Hz, 2H), 3.58 (t, J = 5.2 Hz, 2H), 3.05 (t, J = 5.3 Hz, 2H), 2.73 (s, 3H). | 297.1 |
| 232 | 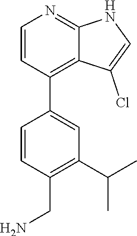 [SMILES: CC(C)c1cc(-c2ccnc3[nH]cc(Cl)c23)ccc1CN] | (4-(3-chloro-1H- pyrrolo[2,3- b]pyridin-4-yl)- 2- isopropylphenyl) methanamine | | 300.1 |
| 233 | 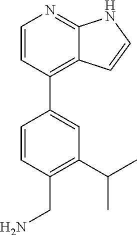 [SMILES: CC(C)c1cc(-c2ccnc3[nH]ccc23)ccc1CN] | (2-isopropyl-4- (1H-pyrrolo[2,3- b]pyridin-4- yl)phenyl) methanamine | | 266.1 |
| 234 | 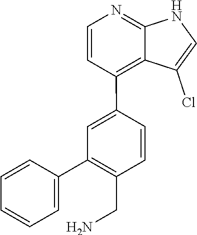 [SMILES: NCc1ccc(-c2ccnc3[nH]cc(Cl)c23)cc1-c1ccccc1] | (5-(3-chloro-1H- pyrrolo[2,3- b]pyridin-4-yl)- [1,1′-biphenyl]- 2- yl)methanamine | ¹H NMR (400 MHz, DMSO-d₆) δ 12.22 (s, 1H), 8.34 (d, J = 4.9 Hz, 1H), 8.22 (s, 3H), 7.81- 7.58 (m, 3H), 7.59-7.33 (m, 6H), 7.13 (d, J = 4.9 Hz, 1H), 4.06 (d, J = 5.8 Hz, 2H). | 334.1 |
| 235 | 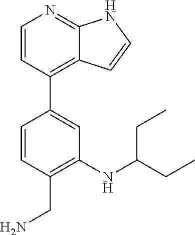 [SMILES: CCC(CC)Nc1cc(-c2ccnc3[nH]ccc23)ccc1CN] | 2- (aminomethyl)- N-(pentan-3-yl)- 5-(1H- pyrrolo[2,3- b]pyridin-4- yl)aniline | ¹H NMR (400 MHz, DMSO-d₆) δ 11.81 (s, 1H), 8.26 (d, J = 4.9 Hz, 1H), 7.98 (s, 3H), 7.64- 7.39 (m, 1H), 7.31 (d, J = 7.6 Hz, 1H), 7.14 (d, J = 5.0 Hz, 1H), 6.97 (d, J = 9.2 Hz, 2H), 6.52 (d, J = 3.3 Hz, 1H), 5.11-5.00 (m, 1H), 4.03 (d, J = 5.8 Hz, 2H), 1.57 (p, J = 7.2 Hz, 4H), 0.91 (t, J = 7.4 Hz, 6H). | 309.1 |
| 236 | 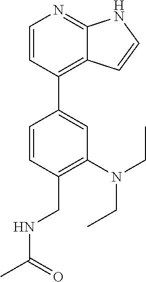 [SMILES: CCN(CC)c1cc(-c2ccnc3[nH]ccc23)ccc1CNC(C)=O] | N-(2- (diethylamino)- 4-(1H- pyrrolo[2,3- b]pyridin-4- yl)benzyl)acetamide | | 337.2 |
| 237 | 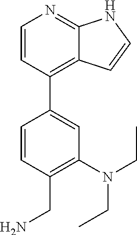 [SMILES: CCN(CC)c1cc(-c2ccnc3[nH]ccc23)ccc1CN] | 2- (aminomethyl)- N,N-diethyl-5- (1H-pyrrolo[2,3- b]pyridin-4- yl)aniline | | 295.1 |
| 238 | 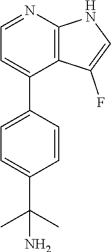 [SMILES: CC(C)(N)c1ccc(-c2ccnc3[nH]cc(F)c23)cc1] | 2-(4-(3-fluoro- 1H-pyrrolo[2,3- b]pyridin-4- yl)phenyl)propan- 2-amine | ¹H NMR (400 MHz, DMSO-d₆) δ 11.70 (s, 1H), 8.43 (s, 3H), 8.34 (d, J = 4.9 Hz, 1H), 7.76 (dd, J = 8.7, 2.7 Hz, 2H), 7.68 (d, J = 8.2 Hz, 2H), 7.56 (d, J = 2.9 Hz, 1H), 7.18 (d, J = 4.9 Hz, 1H), 1.69 (s, 6H). | 270.0 |
| 239 | 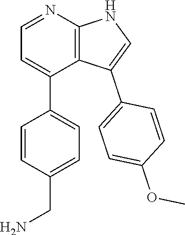 [SMILES: COc1ccc(-c2c[nH]c3nccc(-c4ccc(CN)cc4)c23)cc1] | (4-(3-(4- methoxyphenyl)- 1H-pyrrolo[2,3- b]pyridin-4- yl)phenyl) methanamine | ¹H NMR (400 MHz, Methanol-d₄) δ 8.25 (d, J = 5.0 Hz, 1H), 7.38 (s, 1H), 7.21-6.96 (m, 5H), 6.86-6.72 (m, 2H), 6.64- 6.37 (m, 2H), 3.71 (s, 3H), 2.01 (s, 2H). | 330.1 |
| 240 | 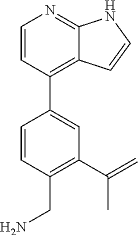 [SMILES: C=C(C)c1cc(-c2ccnc3[nH]ccc23)ccc1CN] | (2-(prop-1-en-2- yl)-4-(1H- pyrrolo[2,3- b]pyridin-4- yl)phenyl) methanamine | ¹H NMR (400 MHz, DMSO-d₆) δ 11.79 (s, 1H), 8.27 (d, J = 5.0 Hz, 2H), 7.79-7.61 (m, 2H), 7.58-7.52 (m, 1H), 7.50 (d, J = 1.7 Hz, 1H), 7.30- 7.06 (m, 1H), 6.57 (dd, J = 3.5, 1.6 Hz, 1H), 5.30 (t, J = 1.8 Hz, 1H), 4.97 (dd, J = 2.1, 1.0 Hz, 1H), 3.89 (s, 2H), 2.10 (dd, J = 1.5, 0.9 Hz, 3H). | 264.1 |
| 241 | 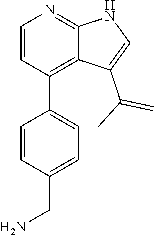 [SMILES: C=C(C)c1c[nH]c2nccc(-c3ccc(CN)cc3)c12] | (4-(3-(prop-1-en- 2-yl)-1H- pyrrolo[2,3- b]pyridin-4- yl)phenyl) methanamine | | 264.1 |
| 242 | 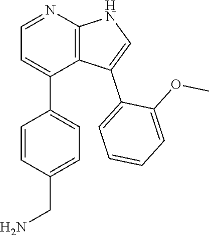 [SMILES: COc1ccccc1-c1c[nH]c2nccc(-c3ccc(CN)cc3)c12] | (4-(3-(2- methoxyphenyl)- 1H-pyrrolo[2,3- b]pyridin-4- yl)phenyl) methanamine | | 330.1 |
| 243 | 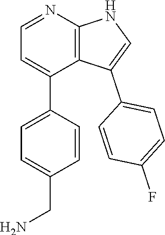 [SMILES: NCc1ccc(-c2ccnc3[nH]cc(-c4ccc(F)cc4)c23)cc1] | (4-(3-(4- fluorophenyl)- 1H-pyrrolo[2,3- b]pyridin-4- yl)phenyl) methanamine | ¹H NMR (400 MHz, DMSO-d₆) δ 12.09 (s, 1H), 8.34 (d, J = 4.8 Hz, 1H), 8.10 (s, 3H), 7.65 (d, J = 2.6 Hz, 1H), 7.32- 7.15 (m, 4H), 7.05 (d, J = 4.9 Hz, 1H), 7.00- 6.64 (m, 4H), 4.02 (d, J = 5.7 Hz, 2H). | 318.1 |
| 244 | 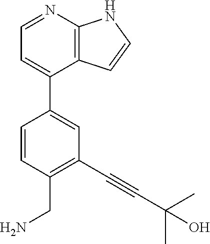 [SMILES: CC(C)(O)C#Cc1cc(-c2ccnc3[nH]ccc23)ccc1CN] | 4-(2- (aminomethyl)- 5-(1H- pyrrolo[2,3- b]pyridin-4- yl)phenyl)-2- methylbut-3-yn- 2-ol | | 306.1 |
| 245 | 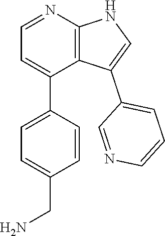 [SMILES: NCc1ccc(-c2ccnc3[nH]cc(-c4cccnc4)c23)cc1] | (4-(3-(pyridin-3- yl)-1H- pyrrolo[2,3- b]pyridin-4- yl)phenyl) methanamine | ¹H NMR (400 MHz, DMSO-d₆) δ 12.36 (s, 1H), 11.86 (s, 2H), 8.39 (d, J = 4.9 Hz, 1H), 8.36 (dd, J = 5.1, 1.6 Hz, 1H), 8.30 (d, J = 5.0 Hz, 2H), 8.24 (d, J = 2.1 Hz, 1H), 7.88 (d, J = 2.7 Hz, 1H), 7.86-7.82 (m, 4H), 7.67-7.61 (m, 4H), 7.57 (dd, J = 3.5, 2.5 Hz, 2H), 7.30-7.18 (m, 6H), 7.11 (d, J = 4.9 Hz, 1H), 6.59 (dd, J = 3.5, 1.9 Hz, 2H), 4.13 (q, J = 5.8 Hz, 4H), | 301.1 |
| | | | 4.01 (q, J = 5.7 Hz, 2H). | |
| 246 | 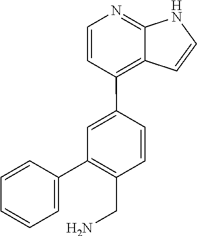 [SMILES: NCc1ccc(-c2ccnc3[nH]ccc23)cc1-c1ccccc1] | (5-(1H- pyrrolo[2,3- b]pyridin-4-yl)- [1,1′-biphenyl]- 2- yl)methanamine | | 300.2 |
| 247 | 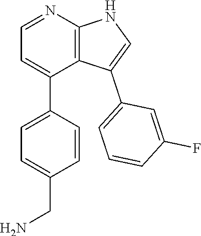 [SMILES: NCc1ccc(-c2ccnc3[nH]cc(-c4cccc(F)c4)c23)cc1] | (4-(3-(3- fluorophenyl)- 1H-pyrrolo[2,3- b]pyridin-4- yl)phenyl) methanamine | ¹H NMR (400 MHz, DMSO-d₆) δ 12.17 (s, 1H), 8.35 (d, J = 4.8 Hz, 1H), 8.10 (s, 3H), 7.74 (d, J = 2.7 Hz, 1H), 7.24 (q, J = 8.0 Hz, 4H), 7.15- 6.95 (m, 2H), 6.87 (t, J = 8.7 Hz, 1H), 6.79- 6.58 (m, 2H), 4.00 (d, J = 5.5 Hz, 2H). | 318.1 |
| 248 | 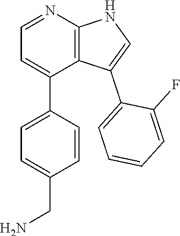 [SMILES: NCc1ccc(-c2ccnc3[nH]cc(-c4ccccc4F)c23)cc1] | (4-(3-(2- fluorophenyl)- 1H-pyrrolo[2,3- b]pyridin-4- yl)phenyl) methanamine | ¹H NMR (400 MHz, DMSO-d₆) δ 12.14 (s, 1H), 8.32 (d, J = 4.8 Hz, 1H), 8.05 (s, 3H), 7.63 (d, J = 2.6 Hz, 1H), 7.20- 7.08 (m, 6H), 7.04 (d, J = 4.9 Hz, 1H), 6.98 (t, J = 7.4 Hz, 1H), 6.78- 6.63 (m, 1H), 3.94 (d, J = 5.7 Hz, 2H). | 318.1 |
| 249 | 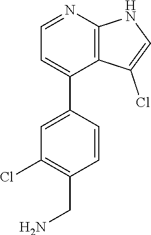 [SMILES: NCc1ccc(-c2ccnc3[nH]cc(Cl)c23)cc1Cl] | (2-chloro-4-(3- chloro-1H- pyrrolo[2,3- b]pyridin-4- yl)phenyl) methanamine | | 292.0 |
| 250 | 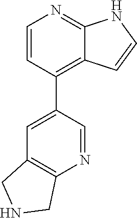 [SMILES: c1cc(-c2cnc3c(c2)CNC3)c2cc[nH]c2n1] | 3-(1H- pyrrolo[2,3- b]pyridin-4-yl)- 6,7-dihydro-5H- pyrrolo[3,4- b]pyridine | ¹H NMR (400 MHz, DMSO-d₆) δ 11.94 (s, 1H), 9.55 (s, 2H), 8.90 (d, J = 2.0 Hz, 1H), 8.33 (d, J = 4.9 Hz, 1H), 8.25 (d, J = 2.0 Hz, 1H), 7.68- 7.53 (m, 1H), 7.27 (d, J = 4.9 Hz, 1H), 6.65 (dd, J = 3.5, 1.9 Hz, 1H), 4.72- 4.67 (m, 2H), 4.62-4.56 (m, 2H). | 237.1 |
| 251 | 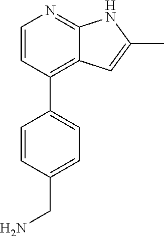 [SMILES: Cc1cc2c(-c3ccc(CN)cc3)ccnc2[nH]1] | (4-(2-methyl- 1H-pyrrolo[2,3- b]pyridin-4- yl)phenyl) methanamine | ¹H NMR (400 MHz, DMSO-d₆) δ 11.72 (s, 1H), 8.19 (d, J = 5.0 Hz, 1H), 7.96-7.74 (m, 2H), 7.62 (d, J = 8.2 Hz, 2H), 7.16 (d, J = 5.1 Hz, 1H), 6.33 (s, 1H), 4.13 (q, J = 5.8 Hz, 2H), 2.43 (s, 3H). | 238.1 |
| 252 | 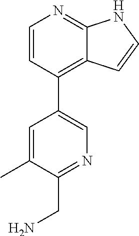 [SMILES: Cc1cc(-c2ccnc3[nH]ccc23)cnc1CN] | (3-methyl-5- (1H-pyrrolo[2,3- b]pyridin-4- yl)pyridin-2- yl)methanamine | ¹H NMR (400 MHz, DMSO-d₆) δ 11.90 (s, 1H), 8.81 (d, J = 2.1 Hz, 1H), 8.32 (d, J = 4.9 Hz, 2H), 8.08 (d, J = 2.1 Hz, 1H), 7.73-7.48 (m, 1H), 7.26 (d, J = 4.9 Hz, 1H), 6.58 (dd, J = 3.5, 1.8 Hz, 1H), 4.29 (d, J = 5.5 Hz, 2H), 2.39 (s, 3H). | 239.1 |
| 253 | 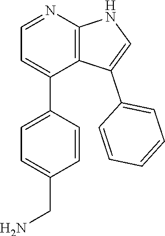 [SMILES: NCc1ccc(-c2ccnc3[nH]cc(-c4ccccc4)c23)cc1] | (4-(3-phenyl-1H- pyrrolo[2,3- b]pyridin-4- yl)phenyl) methanamine | ¹H NMR (400 MHz, DMSO-d₆) δ 12.02 (s, 1H), 8.30 (d, J = 4.8 Hz, 1H), 7.60 (s, 1H), 7.21- 6.94 (m, 8H), 6.94-6.67 (m, 2H), 3.68 (s, 2H). | 300.1 |
| 254 | 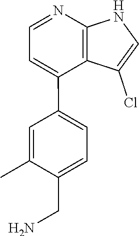 [SMILES: Cc1cc(-c2ccnc3[nH]cc(Cl)c23)ccc1CN] | (4-(3-chloro-1H- pyrrolo[2,3- b]pyridin-4-yl)- 2- methylphenyl) methanamine | ¹H NMR (400 MHz, DMSO-d₆) δ 12.20 (s, 1H), 8.33 (dd, J = 4.8, 1.0 Hz, 1H), 8.19 (s, 4H), 7.72 (d, J = 2.8 Hz, 1H), 7.59-7.40 (m, 3H), 7.03 (dd, J = 4.9, 1.0 Hz, 1H), 4.12 (d, J = 5.9 Hz, 2H), 2.43 (s, 3H). | 272.0 |
| 255 | 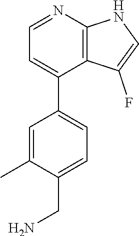 [SMILES: Cc1cc(-c2ccnc3[nH]cc(F)c23)ccc1CN] | (4-(3-fluoro-1H- pyrrolo[2,3- b]pyridin-4-yl)- 2- methylphenyl) methanamine | ¹H NMR (400 MHz, DMSO-d₆) δ 11.68 (s, 1H), 8.33 (d, J = 4.9 Hz, 1H), 8.16 (s, 3H), 7.66- 7.43 (m, 4H), 7.15 (d, J = 4.9 Hz, 1H), 4.12 (q, J = 5.8 Hz, 2H), 2.44 (s, 3H). | 256.2 |
| 256 | 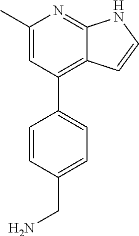 [SMILES: Cc1cc(-c2ccc(CN)cc2)c2cc[nH]c2n1] | (4-(6-methyl- 1H-pyrrolo[2,3- b]pyridin-4- yl)phenyl) methanamine | ¹H NMR (400 MHz, Methanol-d₄) δ 7.76- 7.66 (m, 2H), 7.48 (dd, J = 8.4, 3.4 Hz, 2H), 7.32 (t, J = 4.1 Hz, 1H), 7.06 (d, J = 3.0 Hz, 1H), 6.58 (d, J= 3.5 Hz, 1H), 3.87 (s, 2H), 2.61 (d, J = 1.9 Hz, 3H). | 238.1 |
| 257 | 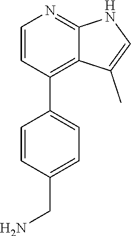 [SMILES: Cc1c[nH]c2nccc(-c3ccc(CN)cc3)c12] | (4-(3-methyl- 1H-pyrrolo[2,3- b]pyridin-4- yl)phenyl) methanamine | ¹H NMR (400 MHz, Methanol-d₄) δ 8.14 (d, J = 5.0 Hz, 1H), 7.53- 7.35 (m, 4H), 7.14 (q, J = 1.1 Hz, 1H), 6.92 (d, J = 5.0 Hz, 1H), 3.88 (s, 2H), 1.91 (d, J = 1.1 Hz, 3H). | 238.1 |
| 258 | 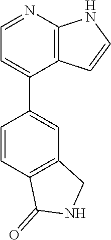 [SMILES: O=C1NCc2cc(-c3ccnc4[nH]ccc34)ccc21] | 5-(1H- pyrrolo[2,3- b]pyridin-4- yl)isoindolin-1- one | ¹H NMR (400 MHz, Methanol-d₄) δ 8.28 (d, J = 5.1 Hz, 1H), 8.03- 7.84 (m, 3H), 7.48 (d, J = 3.6 Hz, 1H), 7.27 (d, J = 5.2 Hz, 1H), 6.74-6.64 (m, 1H), 4.58 (s, 2H). | 250.0 |
| 259 | 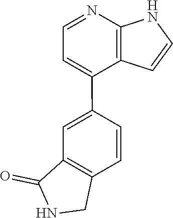 [SMILES: O=C1NCc2ccc(-c3ccnc4[nH]ccc34)cc21] | 6-(1H- pyrrolo[2,3- b]pyridin-4- yl)isoindolin-1- one | | 250.0 |
| 260 | 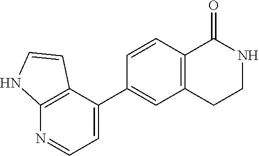 [SMILES: O=C1NCCc2cc(-c3ccnc4[nH]ccc34)ccc21] | 6-(1H- pyrrolo[2,3- b]pyridin-4-yl)- 3,4- dihydroisoquinol in-1(2H)-one | ¹H NMR (400 MHz, Methanol-d₄) δ 8.27 (d, J = 5.1 Hz, 1H), 8.09 (d, J = 8.0 Hz, 1H), 7.77 (d, J = 8.2 Hz, 1H), 7.71 (s, 1H), 7.48 (d, J = 3.6 Hz, 1H), 7.25 (d, J = 5.1 Hz, 1H), 6.69 (d, J = 3.6 Hz, 1H), 3.58 (t, J = 6.6 Hz, 2H), 3.11 (t, J = 6.6 Hz, 2H). | 264.1 |
| 261 | 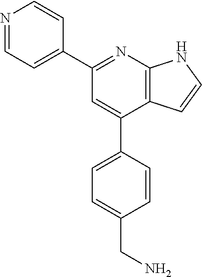 [SMILES: NCc1ccc(-c2cc(-c3ccncc3)nc3[nH]ccc23)cc1] | (4-(6-(pyridin-4- yl)-1H- pyrrolo[2,3- b]pyridin-4- yl)phenyl) methanamine | ¹H NMR (400 MHz, DMSO-d₆) δ 12.18 (s, 1H), 8.95-8.64 (m, 2H), 8.57-8.39 (m, 2H), 8.26 (bs, 3H), 8.06 (s, 1H), 8.01-7.92 (m, 2H), 7.76 (dd, J = 3.5, 2.5 Hz, 1H), 7.68 (s, 1H), 6.68 (dd, J = 3.5, 1.8 Hz, 1H), 4.15 (q, J = 5.8 Hz, 2H). | 301.1 |
| 262 | 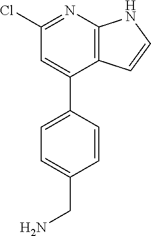 [SMILES: NCc1ccc(-c2cc(Cl)nc3[nH]ccc23)cc1] | (4-(6-chloro-1H- pyrrolo[2,3- b]pyridin-4- yl)phenyl) methanamine | ¹H NMR (400 MHz, Chloroform-d) δ 9.46 (s, 1H), 7.82-7.57 (m, 2H), 7.58-7.44 (m, 2H), 7.21 (s, 1H), 6.68 (dd, J = 3.6, 2.1 Hz, 1H), 3.98 (s, 2H). | 258.0 |
| 263 | 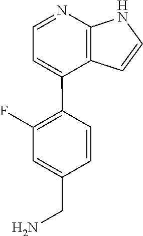 [SMILES: NCc1ccc(-c2ccnc3[nH]ccc23)c(F)c1] | (3-fluoro-4-(1H- pyrrolo[2,3- b]pyridin-4- yl)phenyl) methanamine | ¹H NMR (400 MHz, Methanol-d₄) δ 8.34 (d, J = 5.4 Hz, 1H), 7.76 (t, J = 7.6 Hz, 1H), 7.55 (d, J = 3.6 Hz, 1H), 7.47 (d, J = 9.3 Hz, 2H), 7.31 (dd, J = 5.3, 1.5 Hz, 1H), 6.51 (dd, J = 3.6, 2.1 Hz, 1H), 4.25 (s, 2H). | 242.1 |
| 264 | 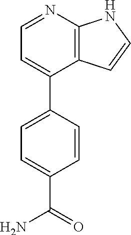 [SMILES: NC(=O)c1ccc(-c2ccnc3[nH]ccc23)cc1] | 4-(1H- pyrrolo[2,3- b]pyridin-4- yl)benzamide | ¹H NMR (400 MHz, Methanol-d₄) δ 8.41 (d, J = 5.8 Hz, 1H), 8.10 (d, J = 8.3 Hz, 2H), 7.94 (d, J = 8.3 Hz, 2H), 7.67 (d, J = 3.6 Hz, 1H), 7.55 (d, J = 5.8 Hz, 1H), 6.90 (d, J = 3.6 Hz, 1H). | 238.1 |
| 265 | 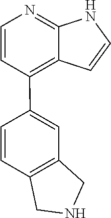 [SMILES: c1cc(-c2ccc3c(c2)CNC3)c2cc[nH]c2n1] | 4-(isoindolin-5- yl)-1H- pyrrolo[2,3- b]pyridine | ¹H NMR (400 MHz, DMSO-d₆) δ 11.84 (s, 1H), 9.43 (s, 2H), 8.28 (d, J = 4.9 Hz, 1H), 7.81 (d, J= 1.5 Hz, 1H), 7.75 (dd, J = 8.0, 1.6 Hz, 1H), 7.60-7.52 (m, 2H), 7.19- 7.16 (m, 1H), 6.61 (dd, J = 3.5, 1.8 Hz, 1H), 4.71- 4.48 (m, 4H). | 236.1 |
| 266 | 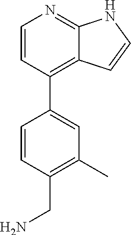 [SMILES: Cc1cc(-c2ccnc3[nH]ccc23)ccc1CN] | (2-methyl-4- (1H-pyrrolo[2,3- b]pyridin-4- yl)phenyl) methanamine | ¹H NMR (400 MHz, DMSO-d₆) δ 11.84 (s, 1H), 8.29 (d, J = 4.9 Hz, 1H), 8.16 (s, 3H), 7.67 (d, J = 7.1 Hz, 2H), 7.62- 7.47 (m, 2H), 7.19 (d, J = 4.9 Hz, 1H), 6.59 (dd, J = 3.5, 1.8 Hz, 1H), 4.12 (q, J = 5.8 Hz, 2H), 2.46 (s, 3H). | 238.1 |
| 267 | 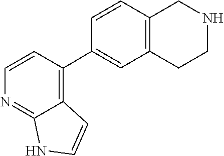 [SMILES: c1cc(-c2ccc3c(c2)CCNC3)c2cc[nH]c2n1] | 6-(1H- pyrrolo[2,3- b]pyridin-4-yl)- 1,2,3,4- tetrahydroiso- quinoline | ¹H NMR (400 MHz, DMSO-d₆) δ 11.86 (s, 1H), 9.04 (s, 2H), 8.29 (d, J = 5.0 Hz, 1H), 7.65 (d, J = 8.2 Hz, 2H), 7.56 (dd, J = 3.5, 2.5 Hz, 1H), 7.40 (d, J = 7.9 Hz, 1H), 7.19 (d, J = 5.0 Hz, 1H), 6.62 (dd, J = 3.5, 1.9 Hz, 1H), 4.56-4.27 (m, 2H), | 250.1 |
| | | | 3.61-3.35 (m, 2H), 3.12 | |
| | | | (t, J = 6.3 Hz, 2H). | |
| 268 | 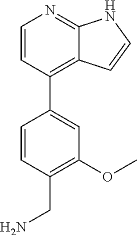 [SMILES: COc1cc(-c2ccnc3[nH]ccc23)ccc1CN] | (2-methoxy-4- (1H-pyrrolo[2,3- b]pyridin-4- yl)phenyl) methanamine | ¹H NMR (400 MHz, DMSO-d₆) δ 11.88 (s, 1H), 8.32 (d, J = 4.9 Hz, 1H), 8.07 (s, 3H), 7.71- 7.50 (m, 2H), 7.42 (dd, J = 6.1, 1.7 Hz, 2H), 7.26 (d, J = 4.9 Hz, 1H), 6.64 (dd, J = 3.5, 1.9 Hz, 1H), 4.08 (q, J = 5.8 Hz, 2H), 3.96 (s, 3H). | 254.1 |
| 269 | 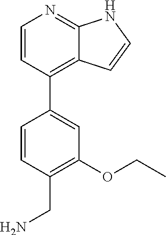 [SMILES: CCOc1cc(-c2ccnc3[nH]ccc23)ccc1CN] | (2-ethoxy-4-(1H- pyrrolo[2,3- b]pyridin-4- yl)phenyl) methanamine | ¹H NMR (400 MHz, DMSO-d₆) δ 11.86 (s, 1H), 8.30 (d, J = 4.9 Hz, 1H), 8.05 (s, 3H), 7.65- 7.48 (m, 2H), 7.39 (d, J = 7.9 Hz, 2H), 7.24 (d, J = 5.0 Hz, 1H), 6.60 (dd, J = 3.5, 1.8 Hz, 1H), 4.23 (q, J = 6.9 Hz, 2H), 4.07 (d, J = 5.8 Hz, 2H), 1.42 (t, J = 6.9 Hz, 3H). | 268.1 |
| 270 | 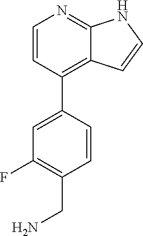 [SMILES: NCc1ccc(-c2ccnc3[nH]ccc23)cc1F] | (2-fluoro-4-(1H- pyrrolo[2,3- b]pyridin-4- yl)phenyl) methanamine | ¹H NMR (400 MHz, DMSO-d₆) δ 11.91 (s, 1H), 8.32 (d, J = 4.9 Hz, 1H), 8.27 (s, 3H), 7.81- 7.68 (m, 3H), 7.68-7.56 (m, 2H), 7.25 (d, J = 5.0 Hz, 1H), 6.61 (dd, J = 3.5, 1.8 Hz, 1H), 4.17 (q, J = 5.8 Hz, 2H). | 242.1 |
| 271 | 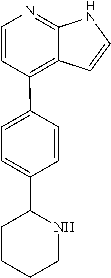 [SMILES: c1cc(-c2ccc(C3CCCCN3)cc2)c2cc[nH]c2n1] | 4-(4-(piperidin- 2-yl)phenyl)-1H- pyrrolo[2,3- b]pyridine | ¹H NMR (400 MHz, DMSO-d₆) δ 11.86 (s, 1H), 8.97 (d, J = 11.4 Hz, 1H), 8.72 (d, J = 11.2 Hz, 1H), 8.30 (d, J = 5.0 Hz, 1H), 8.02-7.78 (m, 2H), 7.66 (d, J = 8.3 Hz, 2H), 7.57 (dd, J = 3.5, 2.5 Hz, 1H), 7.21 (d, J = 5.0 Hz, 1H), 6.60 (dd, J = 3.5, 1.8 Hz, 1H), 4.33 (t, J = 11.3 Hz, 1H), 3.38 (d, J = 12.6 Hz, 1H), 3.15-3.05 (m, 1H), 2.07-1.97 (m, 1H), 1.97-1.87 (m, 2H), 1.86-1.61 (m, 2H). | 278.1 |
| 272 | 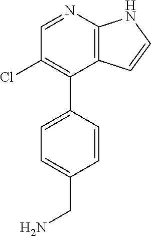 [SMILES: NCc1ccc(-c2c(Cl)cnc3[nH]ccc23)cc1] | (4-(5-chloro-1H- pyrrolo[2,3- b]pyridin-4- yl)phenyl) methanamine | ¹H NMR (400 MHz, DMSO-d₆) δ 12.05 (s, 1H), 8.34 (s, 1H), 8.17 (s, 3H), 7.71-7.53 (m, 5H), 6.15 (dd, J = 3.5, 1.8 Hz, 1H), 4.15 (s, 2H). | 258.0 |
| 273 | 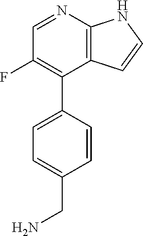 [SMILES: NCc1ccc(-c2c(F)cnc3[nH]ccc23)cc1] | (4-(5-fluoro-1H- pyrrolo[2,3- b]pyridin-4- yl)phenyl) methanamine | ¹H NMR (400 MHz, DMSO-d₆) δ 11.96 (s, 1H), 8.31 (d, J = 3.0 Hz, 1H), 8.22 (s, 3H), 7.74 (dd, J = 8.3, 1.6 Hz, 2H), 7.69-7.61 (m, 3H), 6.39 (dd, J = 3.5, 1.9 Hz, 1H), 4.21-4.09 (m, 2H). | 242.0 |
| 274 | 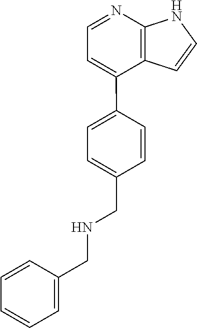 [SMILES: c1ccc(CNCc2ccc(-c3ccnc4[nH]ccc34)cc2)cc1] | N-(4-(1H- pyrrolo[2,3- b]pyridin-4- yl)benzyl)-1- phenylmethanamine | | 314.1 |
| 275 | 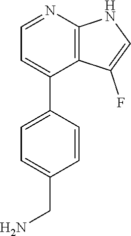 [SMILES: NCc1ccc(-c2ccnc3[nH]cc(F)c23)cc1] | (4-(3-fluoro-1H- pyrrolo[2,3- b]pyridin-4- yl)phenyl) methanamine | ¹H NMR (400 MHz, DMSO-d₆) δ 11.67 (s, 1H), 8.31 (d, J = 4.9 Hz, 1H), 8.19 (s, 3H), 7.70 (dd, J = 8.2, 2.8 Hz, 2H), 7.64-7.56 (m, 2H), 7.56- 7.43 (m, 1H), 7.14 (d, J = 4.9 Hz, 1H), 4.11 (q, J = 5.8 Hz, 2H). | 242.1 |
| 276 | 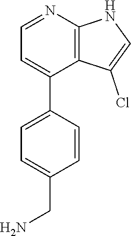 [SMILES: NCc1ccc(-c2ccnc3[nH]cc(Cl)c23)cc1] | (4-(3-chloro-1H- pyrrolo[2,3- b]pyridin-4- yl)phenyl) methanamine | ¹H NMR (400 MHz, DMSO-d₆) δ 12.20 (s, 1H), 8.34 (d, J = 4.9 Hz, 1H), 8.22 (s, 3H), 7.72 (d, J = 2.8 Hz, 1H), 7.67- 7.46 (m, 4H), 7.04 (d, J = 4.9 Hz, 1H), 4.14 (q, J = 5.8 Hz, 2H). | 258.0 |
| 277 | 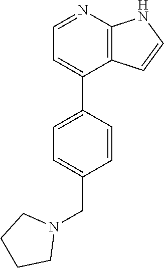 [SMILES: c1cc(-c2ccc(CN3CCCC3)cc2)c2cc[nH]c2n1] | 4-(4-(pyrrolidin- 1- ylmethyl)phenyl)- 1H-pyrrolo[2,3- b]pyridine | ¹H NMR (400 MHz, DMSO-d₆) δ 11.87 (s, 1H), 8.32 (d, J = 5.0 Hz, 1H), 8.08-7.85 (m, 2H), 7.71 (d, J = 8.2 Hz, 2H), 7.59 (dd, J = 3.5, 2.4 Hz, 1H), 7.24 (d, J = 5.0 Hz, 1H), 6.63 (dd, J = 3.5, 1.8 Hz, 1H), 4.46 (d, J = 5.6 Hz, 2H), 3.50-3.41 (m, 2H), 3.22-3.11 (m, 2H), 2.13-2.02 (m, 2H), 1.97-1.84 (m, 2H). | 278.1 |
| 278 | 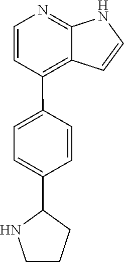 [SMILES: c1cc(-c2ccc(C3CCCN3)cc2)c2cc[nH]c2n1] | 4-(4-(pyrrolidin- 2-yl)phenyl)-1H- pyrrolo[2,3- b]pyridine | ¹H NMR (400 MHz, DMSO-d₆) δ 11.98- 11.81 (m, 1H), 9.47 (s, 1H), 8.78 (s, 1H), 8.32 (d, J = 5.0 Hz, 1H), 7.97- 7.84 (m, 2H), 7.80- 7.65 (m, 2H), 7.60 (dd, J = 3.5, 2.5 Hz, 1H), 7.24 (d, J = 4.9 Hz, 1H), 6.62 (dd, J = 3.5, 1.8 Hz, 1H), 4.68 (p, J = 8.5 Hz, 1H), 3.39 (td, J = 9.2, 7.5, 5.0 Hz, 2H), 2.49-2.30 (m, 2H), 2.28-1.81 (m, 2H). | 264.1 |
| 279 | 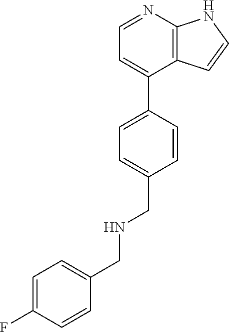 [SMILES: Fc1ccc(CNCc2ccc(-c3ccnc4[nH]ccc34)cc2)cc1] | N-(4-(1H- pyrrolo[2,3- b]pyridin-4- yl)benzyl)-1-(4- fluorophenyl) methanamine | ¹H NMR (400 MHz, DMSO-d₆) δ 11.85 (s, 1H), 9.26 (s, 2H), 8.30 (d, J = 5.0 Hz, 1H), 7.95- 7.76 (m, 2H), 7.66 (d, J = 8.3 Hz, 2H), 7.57 (td, J = 5.6, 2.4 Hz, 3H), 7.30 (t, J = 8.9 Hz, 2H), 7.25- 7.16 (m, 1H), 6.59 (dd, J = 3.4, 1.9 Hz, 1H), 4.29- 4.21 (m, 4H). | 332.1 |
| 280 | 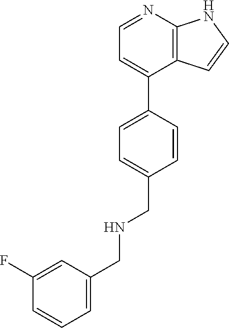 [SMILES: Fc1cccc(CNCc2ccc(-c3ccnc4[nH]ccc34)cc2)c1] | N-(4-(1H- pyrrolo[2,3- b]pyridin-4- yl)benzyl)-1-(3- fluorophenyl) methanamine | ¹H NMR (400 MHz, DMSO-d₆) δ 11.86 (s, 1H), 9.30 (s, 2H), 8.30 (d, J = 4.9 Hz, 1H), 7.94- 7.82 (m, 2H), 7.73- 7.63 (m, 2H), 7.57 (dd, J = 3.5, 2.5 Hz, 1H), 7.51 (td, J = 8.0, 6.1 Hz, 1H), 7.42-7.38 (m, 1H), 7.36 (dt, J = 7.7, 1.2 Hz, 1H), 7.32-7.25 (m, 1H), 7.21 (d, J = 4.9 Hz, 1H), 6.59 (dd, J = 3.5, 1.9 Hz, 1H), 4.28 (t, J = 5.9 Hz, 4H). | 332.1 |
| 281 | 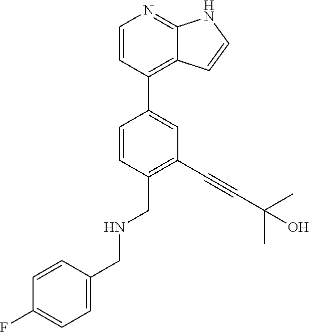 [SMILES: CC(C)(O)C#Cc1cc(-c2ccnc3[nH]ccc23)ccc1CNCc1ccc(F)cc1] | 4-(2-(((4- fluorobenzyl)amino) methyl)-5- (1H-pyrrolo[2,3- b]pyridin-4- yl)phenyl)-2- methylbut-3-yn- 2-ol | ¹H NMR (400 MHz, Methanol-d₄) δ 8.40 (d, J = 5.4 Hz, 1H), 7.98 (dd, J = 6.7, 1.8 Hz, 1H), 7.96- 7.84 (m, 1H), 7.77 (t, J = 7.4 Hz, 1H), 7.67 (dd, J = 6.7, 3.6 Hz, 1H), 7.58 (ddd, J = 7.2, 5.3, 3.4 Hz, 1H), 7.56-7.43 (m, 1H), 7.32-7.15 (m, 2H), 6.88- 6.72 (m, 1H), 4.49 (s, 2H), 4.39 (s, 2H), 1.59 (d, J = 1.5 Hz, 6H). | 414.2 |
| 282 | 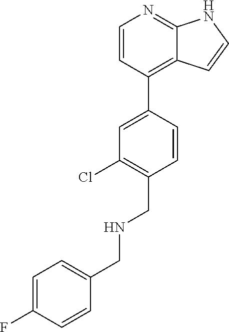 [SMILES: Fc1ccc(CNCc2ccc(-c3ccnc4[nH]ccc34)cc2Cl)cc1] | N-(2-chloro-4- (1H-pyrrolo[2,3- b]pyridin-4- yl)benzyl)-1-(4- fluorophenyl) methanamine | ¹H NMR (400 MHz, DMSO-d₆) δ 11.92 (s, 1H), 8.31 (d, J = 4.9 Hz, 1H), 7.90 (d, J = 1.8 Hz, 1H), 7.86 (dd, J = 8.0, 1.8 Hz, 1H), 7.78 (d, J = 8.0 Hz, 1H), 7.64-7.58 (m, 3H), 7.36-7.28 (m, 2H), 7.24 (d, J = 4.9 Hz, 1H), 6.57 (dd, J = 3.5, 1.9 Hz, 1H), 4.39-4.31 (m, 4H). | 366.1 |
| 283 | 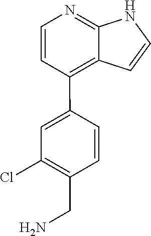 [SMILES: NCc1ccc(-c2ccnc3[nH]ccc23)cc1Cl] | (2-chloro-4-(1H- pyrrolo[2,3- b]pyridin-4- yl)phenyl) methanamine | ¹H NMR (400 MHz, DMSO-d₆) δ 11.96 (s, 1H), 8.56-8.15 (m, 5H), 7.95-7.80 (m, 2H), 7.75 (d, J = 8.1 Hz, 1H), 7.68- 7.62 (m, 1H), 7.27 (d, J = 4.9 Hz, 1H), 6.59 (dd, J = 3.5, 1.9 Hz, 1H), 4.25 (d, J = 5.7 Hz, 2H). | 258.0 |
| 284 | 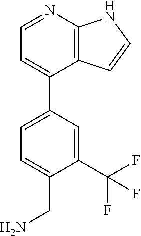 [SMILES: NCc1ccc(-c2ccnc3[nH]ccc23)cc1C(F)(F)F] | (4-(1H- pyrrolo[2,3- b]pyridin-4-yl)- 2- (trifluoromethyl) phenyl) methanamine | ¹H NMR (400 MHz, DMSO-d₆) δ 11.96 (s, 1H), 8.42 (s, 3H), 8.35 (d, J= 4.9 Hz, 1H), 8.30- 8.17 (m, 1H), 8.12 (d, J = 1.8 Hz, 1H), 7.90 (d, J = 8.1 Hz, 1H), 7.63 (dd, J = 3.5, 2.5 Hz, 1H), 7.32 (d, J = 5.0 Hz, 1H), 6.58 (dd, J = 3.5, 1.9 Hz, 1H), 4.30 (d, J = 5.5 Hz, 2H). | 292.1 |
| 285 | 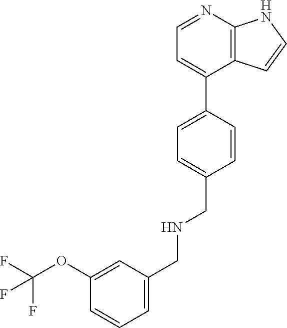 [SMILES: FC(F)(F)Oc1cccc(CNCc2ccc(-c3ccnc4[nH]ccc34)cc2)c1] | N-(4-(1H- pyrrolo[2,3- b]pyridin-4- yl)benzyl)-1-(3- (trifluoromethox y)phenyl) methanamine | ¹H NMR (400 MHz, DMSO-d₆) δ 11.85 (s, 1H), 9.41 (s, 2H), 8.30 (d, J = 4.9 Hz, 1H), 7.95- 7.78 (m, 2H), 7.78- 7.63 (m, 3H), 7.63-7.55 (m, 2H), 7.55-7.43 (m, 2H), 7.20 (d, J = 5.0 Hz, 1H), 6.58 (dd, J = 3.5, 1.9 Hz, 1H), 4.37 (d, J = 5.1 Hz, 2H), 4.29 (s, 2H). | 398.1 |
| 286 | 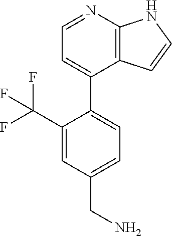 [SMILES: NCc1ccc(-c2ccnc3[nH]ccc23)c(C(F)(F)F)c1] | (4-(1H- pyrrolo[2,3- b]pyridin-4-yl)- 3- (trifluoromethyl) phenyl) methanamine | ¹H NMR (400 MHz, DMSO-d₆) δ 11.87 (s, 1H), 8.28 (d, J = 4.9 Hz, 1H), 8.07 (d, J = 1.8 Hz, 1H), 7.84 (d, J = 7.9 Hz, 1H), 7.58 (d, J = 7.9 Hz, 1H), 7.50 (dd, J = 3.5, 2.5 Hz, 1H), 6.93 (dd, J = 4.9, 0.8 Hz, 1H), 6.00 (dd, J = 3.5, 1.8 Hz, 1H), 4.25 (q, J = 5.8 Hz, 2H). | 292.0 |
| 287 | 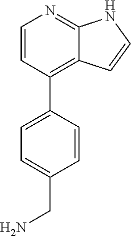 [SMILES: NCc1ccc(-c2ccnc3[nH]ccc23)cc1] | (4-(1H- pyrrolo[2,3- b]pyridin-4- yl)phenyl) methanamine | ¹H NMR (400 MHz, Methanol-d₄) δ 8.29- 8.12 (m, 1H), 7.82-7.68 (m, 2H), 7.57-7.46 (m, 2H), 7.43 (t, J = 3.5 Hz, 1H), 7.26-7.11 (m, 1H), 6.74-6.60 (m, 1H), 3.95- 3.81 (m, 2H). | 224.1 |

### Example 4: LATS Inhibitors: Biological Evaluation

### In Vitro Kinase Inhibition Assays

Biochemical kinase inhibitory data was obtained for various exemplary compounds prepared according to the above. Compounds were evaluated using the RBC HotSpot Kinase Assay Protocol (Anastassiadis T, et al. Comprehensive assay of kinase catalytic activity reveals features of kinase inhibitor selectivity. Nat Biotechnol. 2011 Oct. 30; 29(11):1039-45), as described below.

The reagents used are as follows: Base Reaction buffer; 20 mM Hepes (pH 7.5), 10 mM MgCl2, 1 mM EGTA, 0.01% Brij35, 0.02 mg/ml BSA, 0.1 mM Na3VO4, 2 mM DTT, 100 DMSO. Required cofactors were added individually to each kinase reaction.

The reaction procedure was as follows:
1) Substrates were prepared in freshly prepared Reaction Buffer.
2) Any required cofactors were delivered to the substrate solution above.
3) Kinase was delivered into the substrate solution and gently mixed.
4) Compounds were delivered in 100% DMSO into the kinase reaction mixture by Acoustic technology (Echo550; nanoliter range), followed by incubation for 20 min at room temp.
5) ³³P-ATP was delivered into the reaction mixture to initiate the reaction.
6) The mixture was incubated for 2 hours at room temperature.
7) Kinase activity was detected by P81 filter-binding method.

Using the above functional assay, the inhibitory activity of the compounds for LATS1/2 versus AKT was characterized. The assays were typically run in a 10-point curve, in singlet, to produce a titration curve from which an IC₅₀ value was obtained. The IC₅₀ values are reported in Table 28.

### Ex Vivo Kinase Inhibition Assays

Kinase cellular potency data were obtained for various exemplary compounds, using the Reaction Biology NanoBRET™ assay protocol described below. The NanoBRET™ assay measures kinase engagement in real time in the context of the intact cell. The assay uses a Kinase-NanoLuc® fusion vector expressing a kinase protein to which a luciferase tag has been added, a cell-permeant fluorescent NanoBRET™ tracer, a NanoLuc® substrate, and an extracellular NanoLuc® inhibitor. Upon expression of the luciferase-tagged kinase, cells will produce a strong BRET signal only in the presence of the NanoBRET™ tracer. The extracellular NanoLuc® inhibitor ensures that the BRET signal observed emanates only from live cells. Because the BRET signal has tight distance constraints, addition of the test compound will decrease the BRET signal if the compound competes with the NanoBRET™ tracer for binding to the kinase domain. Under the appropriate tracer conditions established by the manufacturer, quantitative intracellular affinity and relative potency can then be determined using Mass Action model equations.

HEK-293 cells were purchased from ATCC. FuGENEHD Transfection Reagent, Kinase-NanoLuc® fusion plasmids, Transfection Carrier DNA, NanoBRET™ Tracers and dilution buffer, NanoBRET™ Nano-Glo Substrate, Extracellular NanoLuc® Inhibitor were obtained from Promega.

Assays were conducted following Promega assay protocol with some modifications as described further below. HEK-293 Cells were transiently transfected with Kinase-NanoLuc® Fusion Vector DNA by FuGENE HD Transfection Reagent. Test compounds were delivered into 384 well assay plate by Echo 550 (Labcyte Inc, Sunnyvale, CA). Transfected cells were harvested and mixed with NanoBRET™ Tracer Reagent and dispensed into 384 well plates and incubated at 37° C. in 5% CO₂ cell culture incubator for 1 hour. The NanoBRET™ Nano-Glo Substrate plus Extracellular NanoLuc® Inhibitor Solution were added into the wells of the assay plate and incubated for 2-3 minutes at room temperature. The donor emission wavelength (460 nm) and acceptor emission wavelength (600 nm) were measured in the EnVision plate reader. The BRET Ratios were calculated. BRET Ratio=[(Acceptor sample÷Donor sample)−(Acceptor no-tracer control÷Donor no-tracer control)]. The IC₅₀ values of compounds were calculated with Prism GraphPad program.

### NanoBRET™ Target Engagement Assay Protocol:

1. Transient Transfection of HEK-293 Cells NanoLuc® Fusion Vector DNA
   1). Cultivate HEK-293 cells (70-80% confluence) appropriately prior to assay. Trypsinize and collect HEK-293 cells.
   2). Prepare lipid: DNA complexes as follows:
      a. Prepare a 10 g/ml solution of DNA in Opti-MEM™ without serum that consists of the following ratios of carrier DNA and DNA encoding NanoLuc® fusion. 9.0 g/mL of Transfection Carrier DNA, 1.0 g/mL of NanoLuc® fusion vector DNA and 1 mL of Opti-MEM™ without phenol red. Mix thoroughly.
      b. Add 30 μl of FuGENE HD Transfection Reagent into each milliliter of DNA mixture to form lipid: DNA complex.
      c. Mix by inversion 10 times.
      d. Incubate at ambient temperature for 20 minutes to allow complexes to form.
   3). In a sterile, conical tube, mix 1 part of lipid: DNA complex with 20 parts of HEK-293 cells in suspension. Mix gently by inversion 5 times.
   4). Dispense cells+lipid: DNA complex into a sterile tissue culture dish and incubate for 22-24 hours.
2. Addition of Test Compounds (dry plate shooting): Each test compound is delivered from the compound source plate to the wells of 384-well white NBS plate by Echo 550.
3. Preparation of Cells with NanoBRET™ Tracer Reagent
   1). Remove medium from dish with transfected HEK-293 cells via aspiration, trypsinize and allow cells to dissociate from the dish.
   2). Neutralize trypsin using medium containing serum and centrifuge at 200× g for 5 minutes to pellet the cells. Adjust the cell density to 2×105 cells/mL in Opti-MEM™ without phenol red.
   3). Prepare Complete 20× NanoBRET™ Tracer Reagent with Tracer Dilution Buffer.
   4). Dispense one part of Complete 20× NanoBRET™ Tracer Reagent to 20 parts of cells in the tube. Mix gently by inversion 10 times.
   5). Dispense cell suspension into white, 384-well NBS plates. Incubate the plate at 37° C., 5% CO₂ for 1 hour.
Note: Prepare a separate set of samples without tracer for background correction steps.
4. NanoBRET™ Assay
   1). Remove plate from incubator and equilibrate to room temperature for 15 minutes.
   2). Prepare 3× Complete Substrate plus Inhibitor Solution in Assay Medium (Opti-MEM™ I Reduced Serum Medium, no phenol red) just before measuring BRET.
   3). Add 3× Complete Substrate plus Inhibitor Solution to each well of the 384-well plate. Incubate for 2-3 minutes at room temperature.
   4). Measure donor emission wavelength (460 nm) and acceptor emission wavelength (600 nm) using the Envision 2104 plate reader.
5. Determination of BRET Ratio: To generate raw BRET ratio values, divide the acceptor emission value (600 nm) by the donor emission value (460 nm) for each sample. To correct for background, subtract the BRET ratio in the absence of tracer (average of no-tracer control samples) from the BRET ratio of each sample.
NanoBRET™ ratio equation: BRET Ratio=(Acceptor sample÷Donor sample)
NanoBRET™ ratio equation, including optional background correction: BRET Ratio=[(Acceptor sample÷Donor sample)−(Acceptor no-tracer control÷Donor no-tracer control)]
Normalized Bret Response equation (%): (BRET Ratio of Compound Treated Sample/BRET Ratio of DMSO Control Sample)*100%
6. Determination of IC₅₀ Values: IC₅₀ curves are plotted and IC₅₀ values are calculated using the GraphPad Prism 4 program based on a sigmoidal dose-response equation.

IC₅₀ values as determined by the assays described above are provided in Table 28. All biological activities are IC₅₀, in nM.

TABLE 28

| | | | | | AKT1 | LATS1 |
|---|---|---|---|---|---|---|
| Compound | Structure | AKT | LATS1 | LATS2 | (Cellular) | (Cellular) |
| 1 | 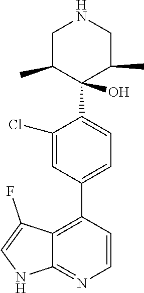 [SMILES: C[C@@H]1CNC[C@H](C)[C@@]1(O)c1ccc(-c2ccnc3[nH]cc(F)c23)cc1Cl] | 15.4 | 11.3 | 14.6 | 21200 | 203 |
| 2 | 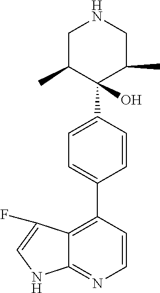 [SMILES: C[C@@H]1CNC[C@H](C)[C@@]1(O)c1ccc(-c2ccnc3[nH]cc(F)c23)cc1] | 147 | 16.0 | 12.5 | >50000 | 550 |
| 3 | 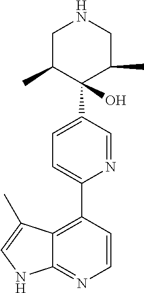 [SMILES: Cc1c[nH]c2nccc(-c3ccc([C@@]4(O)[C@H](C)CNC[C@@H]4C)cn3)c12] | 4409 | 72.3 | 49.6 | | |
| 4 | 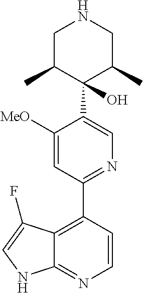 [SMILES: COc1cc(-c2ccnc3[nH]cc(F)c23)ncc1[C@@]1(O)[C@H](C)CNC[C@@H]1C] | 4159 | 18.2 | 56.7 | | |
| 5 | 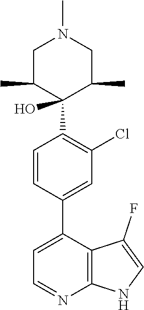 [SMILES: C[C@@H]1CN(C)C[C@H](C)[C@@]1(O)c1ccc(-c2ccnc3[nH]cc(F)c23)cc1Cl] | 67 | 9.2 | 12.5 | | |
| 6 | 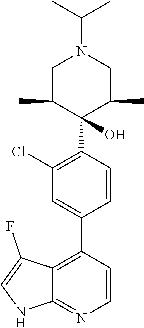 [SMILES: CC(C)N1C[C@@H](C)[C@](O)(c2ccc(-c3ccnc4[nH]cc(F)c34)cc2Cl)[C@@H](C)C1] | 90.9 | 1.3 | 1.4 | | |
| 7 | 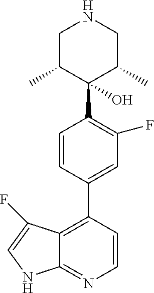 [SMILES: C[C@@H]1CNC[C@H](C)[C@@]1(O)c1ccc(-c2ccnc3[nH]cc(F)c23)cc1F] | 192 | 15.9 | 6.3 | 8640 | 247 |
| 8 | 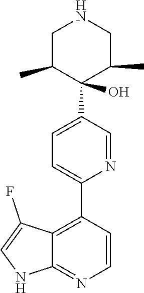 [SMILES: C[C@@H]1CNC[C@H](C)[C@@]1(O)c1ccc(-c2ccnc3[nH]cc(F)c23)nc1] | 3332 | 53 | 203 | 8640 | 60 |
| 9 | 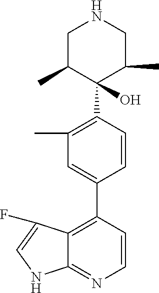 [SMILES: Cc1cc(-c2ccnc3[nH]cc(F)c23)ccc1[C@@]1(O)[C@H](C)CNC[C@@H]1C] | 89.2 | 9.4 | 6.6 | 15230 | 44.6 |
| 10 | 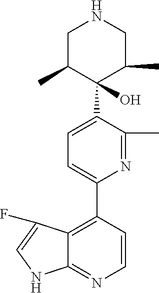 [SMILES: Cc1nc(-c2ccnc3[nH]cc(F)c23)ccc1[C@@]1(O)[C@H](C)CNC[C@@H]1C] | 1718 | 23.7 | 20.3 | | |
| 11 | 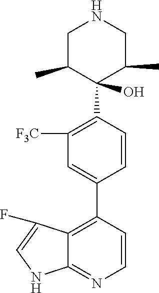 [SMILES: C[C@@H]1CNC[C@H](C)[C@@]1(O)c1ccc(-c2ccnc3[nH]cc(F)c23)cc1C(F)(F)F] | 143 | 11.1 | 10.7 | | |
| 12 | 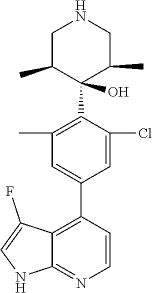 [SMILES: Cc1cc(-c2ccnc3[nH]cc(F)c23)cc(Cl)c1[C@@]1(O)[C@H](C)CNC[C@@H]1C] | 10.1 | 200.7 | 81.2 | | |
| 13 | 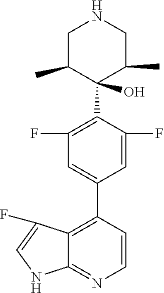 [SMILES: C[C@@H]1CNC[C@H](C)[C@@]1(O)c1c(F)cc(-c2ccnc3[nH]cc(F)c23)cc1F] | 362 | 8.5 | 7.2 | 16100 | 1450 |
| 14 | 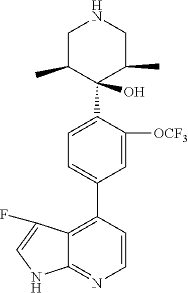 [SMILES: C[C@@H]1CNC[C@H](C)[C@@]1(O)c1ccc(-c2ccnc3[nH]cc(F)c23)cc1OC(F)(F)F] | 111.6 | 5.4 | 5.5 | 4950 | 2130 |
| 15 | 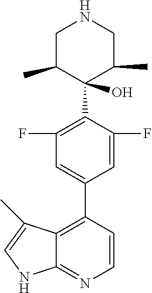 [SMILES: Cc1c[nH]c2nccc(-c3cc(F)c([C@@]4(O)[C@H](C)CNC[C@@H]4C)c(F)c3)c12] | 362 | 8.5 | 7.2 | | |
| 16 | 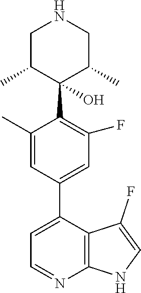 [SMILES: Cc1cc(-c2ccnc3[nH]cc(F)c23)cc(F)c1[C@@]1(O)[C@H](C)CNC[C@@H]1C] | 201 | 7.1 | 10.0 | | |
| 17 | 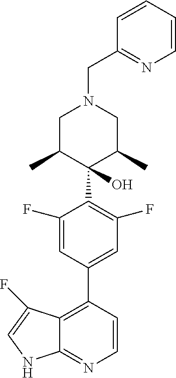 [SMILES: C[C@@H]1CN(Cc2ccccn2)C[C@H](C)[C@@]1(O)c1c(F)cc(-c2ccnc3[nH]cc(F)c23)cc1F] | 1074 | 10.9 | 11.9 | 24100 | 409 |
| 18 | 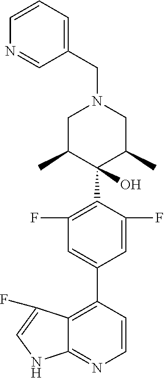 [SMILES: C[C@@H]1CN(Cc2cccnc2)C[C@H](C)[C@@]1(O)c1c(F)cc(-c2ccnc3[nH]cc(F)c23)cc1F] | NA | NA | NA | | |
| 19 | 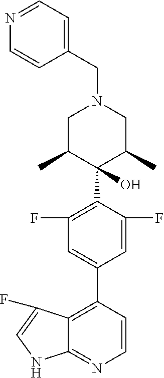 [SMILES: C[C@@H]1CN(Cc2ccncc2)C[C@H](C)[C@@]1(O)c1c(F)cc(-c2ccnc3[nH]cc(F)c23)cc1F] | 1020 | 11.0 | 9.2 | | |
| 20 | 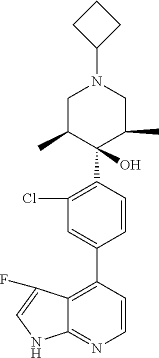 [SMILES: C[C@@H]1CN(C2CCC2)C[C@H](C)[C@@]1(O)c1ccc(-c2ccnc3[nH]cc(F)c23)cc1Cl] | 194.6 | 2.6 | 2.7 | | |
| 21 | 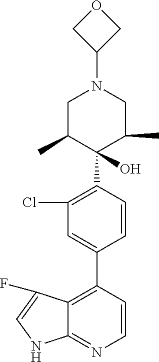 [SMILES: C[C@@H]1CN(C2COC2)C[C@H](C)[C@@]1(O)c1ccc(-c2ccnc3[nH]cc(F)c23)cc1Cl] | 249.2 | 3.0 | 2.8 | | |
| 22 | 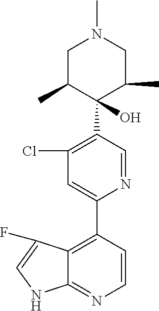 [SMILES: C[C@@H]1CN(C)C[C@H](C)[C@@]1(O)c1cnc(-c2ccnc3[nH]cc(F)c23)cc1Cl] | 505 | 1.1 | 4.1 | 2510 | 505 |
| 23 | 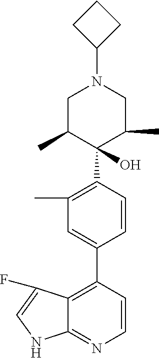 [SMILES: Cc1cc(-c2ccnc3[nH]cc(F)c23)ccc1[C@@]1(O)[C@H](C)CN(C2CCC2)C[C@@H]1C] | 51 | 2.5 | 2.3 | 2970 | 87 |
| 24 | 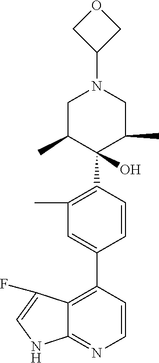 [SMILES: Cc1cc(-c2ccnc3[nH]cc(F)c23)ccc1[C@@]1(O)[C@H](C)CN(C2COC2)C[C@@H]1C] | 374 | 5.7 | 5.7 | 56200 | 127 |
| 25 | 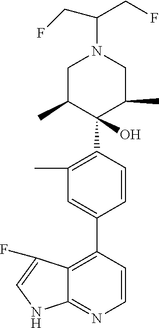 [SMILES: Cc1cc(-c2ccnc3[nH]cc(F)c23)ccc1[C@@]1(O)[C@H](C)CN(C(CF)CF)C[C@@H]1C] | | | | | |
| 26 | 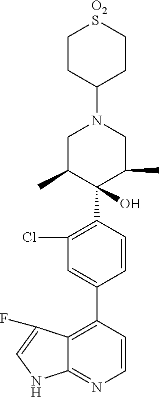 [SMILES: C[C@@H]1CN(C2CCS(=O)(=O)CC2)C[C@H](C)[C@@]1(O)c1ccc(-c2ccnc3[nH]cc(F)c23)cc1Cl] | 18.8 | 0.73 | 0.51 | | |
| 27 | 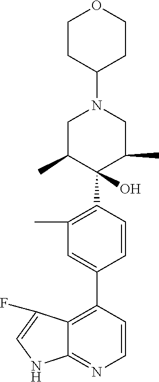 [SMILES: Cc1cc(-c2ccnc3[nH]cc(F)c23)ccc1[C@@]1(O)[C@H](C)CN(C2CCOCC2)C[C@@H]1C] | 47.9 | 1.7 | 2.2 | | |
| 28 | 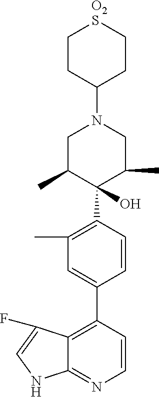 [SMILES: Cc1cc(-c2ccnc3[nH]cc(F)c23)ccc1[C@@]1(O)[C@H](C)CN(C2CCS(=O)(=O)CC2)C[C@@H]1C] | 62.8 | 9.0 | 12.1 | 19800 | 367 |
| 29 | 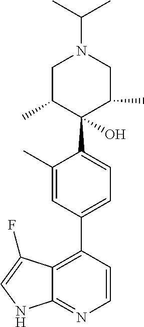 [SMILES: Cc1cc(-c2ccnc3[nH]cc(F)c23)ccc1[C@@]1(O)[C@H](C)CN(C(C)C)C[C@@H]1C] | 43.5 | 1.3 | 2.2 | 17000 | 86 |
| 30 | 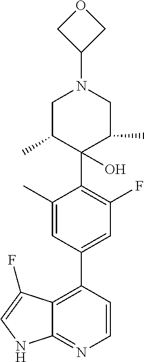 [SMILES: Cc1cc(-c2ccnc3[nH]cc(F)c23)cc(F)c1C1(O)[C@H](C)CN(C2COC2)C[C@@H]1C] | 698 | 5.9 | 9.0 | >30000 | 58.2 |
| 31 | 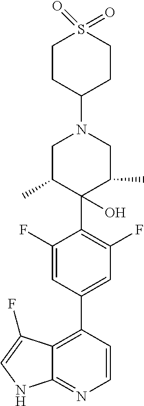 [SMILES: C[C@@H]1CN(C2CCS(=O)(=O)CC2)C[C@H](C)C1(O)c1c(F)cc(-c2ccnc3[nH]cc(F)c23)cc1F] | 129 | 6.7 | 6.3 | 39400 | 1190 |
| 32 | 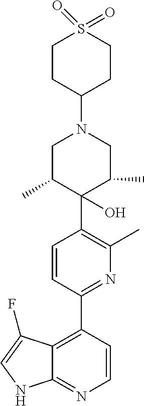 [SMILES: Cc1nc(-c2ccnc3[nH]cc(F)c23)ccc1C1(O)[C@H](C)CN(C2CCS(=O)(=O)CC2)C[C@@H]1C] | | | | | |
| 33 | 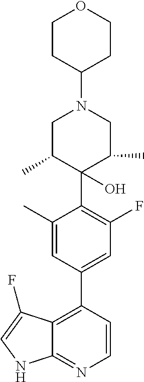 [SMILES: Cc1cc(-c2ccnc3[nH]cc(F)c23)cc(F)c1C1(O)[C@H](C)CN(C2CCOCC2)C[C@@H]1C] | 474 | 3.4 | 1.9 | | |
| 34 | 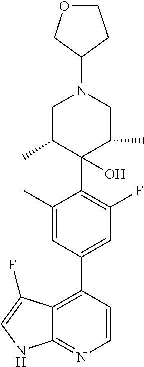 [SMILES: Cc1cc(-c2ccnc3[nH]cc(F)c23)cc(F)c1C1(O)[C@H](C)CN(C2CCOC2)C[C@@H]1C] | 908 | 14.3 | 8.3 | | |
| 35 | 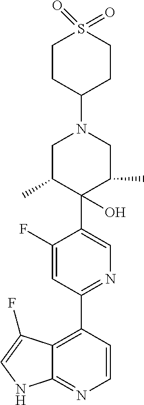 [SMILES: C[C@@H]1CN(C2CCS(=O)(=O)CC2)C[C@H](C)C1(O)c1cnc(-c2ccnc3[nH]cc(F)c23)cc1F] | 2077 | 99.4 | 63.4 | | |
| 36 | 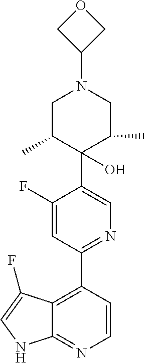 [SMILES: C[C@@H]1CN(C2COC2)C[C@H](C)C1(O)c1cnc(-c2ccnc3[nH]cc(F)c23)cc1F] | >1000 | 98.5 | 79.6 | | |
| 37 | 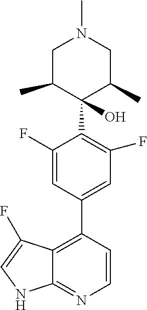 [SMILES: C[C@@H]1CN(C)C[C@H](C)[C@@]1(O)c1c(F)cc(-c2ccnc3[nH]cc(F)c23)cc1F] | 477 | 10.1 | 8.6 | | |
| 38 | 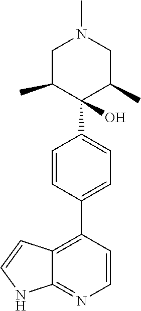 [SMILES: C[C@@H]1CN(C)C[C@H](C)[C@@]1(O)c1ccc(-c2ccnc3[nH]ccc23)cc1] | 181 | 28 | 27 | | |
| 39 | 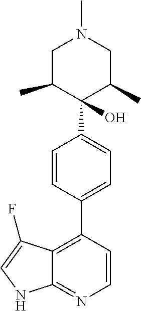 [SMILES: C[C@@H]1CN(C)C[C@H](C)[C@@]1(O)c1ccc(-c2ccnc3[nH]cc(F)c23)cc1] | 213 | 19.5 | 24.7 | >50000 | 19000 |
| 40 | 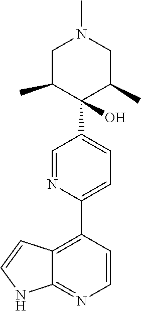 [SMILES: C[C@@H]1CN(C)C[C@H](C)[C@@]1(O)c1ccc(-c2ccnc3[nH]ccc23)nc1] | 1542 | 99 | 293 | | |
| 41 | 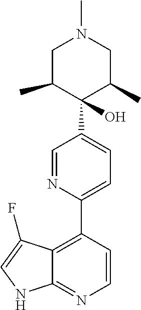 [SMILES: C[C@@H]1CN(C)C[C@H](C)[C@@]1(O)c1ccc(-c2ccnc3[nH]cc(F)c23)nc1] | 4142 | 61 | 268 | 8640 | 115 |
| 42 | 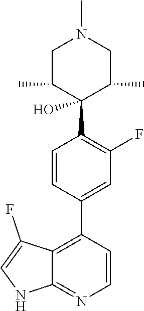 [SMILES: C[C@@H]1CN(C)C[C@H](C)[C@@]1(O)c1ccc(-c2ccnc3[nH]cc(F)c23)cc1F] | 159 | 9.7 | 6.7 | | |
| 43 | 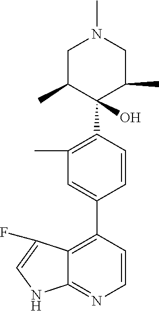 [SMILES: Cc1cc(-c2ccnc3[nH]cc(F)c23)ccc1[C@@]1(O)[C@H](C)CN(C)C[C@@H]1C] | 63.5 | 3.2 | 2.9 | | |
| 44 | 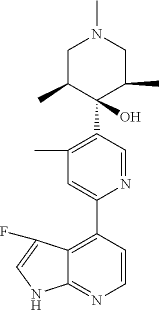 [SMILES: Cc1cc(-c2ccnc3[nH]cc(F)c23)ncc1[C@@]1(O)[C@H](C)CN(C)C[C@@H]1C] | 1610 | 8.9 | 19.6 | | |
| 45 | 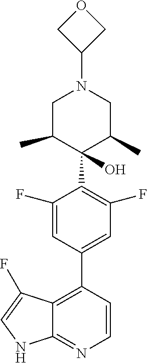 [SMILES: C[C@@H]1CN(C2COC2)C[C@H](C)[C@@]1(O)c1c(F)cc(-c2ccnc3[nH]cc(F)c23)cc1F] | 1173 | 3.4 | 9.8 | >30000 | 813 |
| 46 | 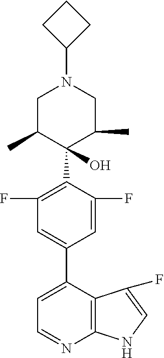 [SMILES: C[C@@H]1CN(C2CCC2)C[C@H](C)[C@@]1(O)c1c(F)cc(-c2ccnc3[nH]cc(F)c23)cc1F] | 358 | 1.7 | 4.0 | 31500 | 462 |
| 47 | 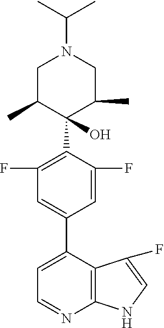 [SMILES: CC(C)N1C[C@@H](C)[C@](O)(c2c(F)cc(-c3ccnc4[nH]cc(F)c34)cc2F)[C@@H](C)C1] | 449 | 4.7 | 11.2 | | |
| 48 | 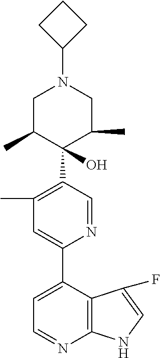 [SMILES: Cc1cc(-c2ccnc3[nH]cc(F)c23)ncc1[C@@]1(O)[C@H](C)CN(C2CCC2)C[C@@H]1C] | 6115 | 2.9 | 16.1 | | 12600 |
| 49 | 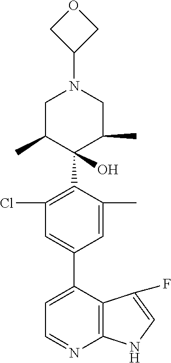 [SMILES: Cc1cc(-c2ccnc3[nH]cc(F)c23)cc(Cl)c1[C@@]1(O)[C@H](C)CN(C2COC2)C[C@@H]1C] | 125 | 697 | 428 | | |
| 50 | 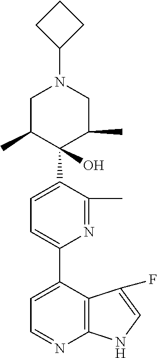 [SMILES: Cc1nc(-c2ccnc3[nH]cc(F)c23)ccc1[C@@]1(O)[C@H](C)CN(C2CCC2)C[C@@H]1C] | 906 | 6.1 | 24.7 | | |
| 51 | 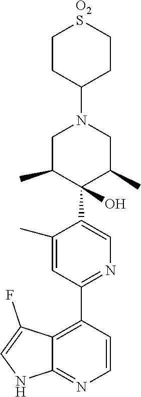 [SMILES: Cc1cc(-c2ccnc3[nH]cc(F)c23)ncc1[C@@]1(O)[C@H](C)CN(C2CCS(=O)(=O)CC2)C[C@@H]1C] | >10000 | 2076 | 4246 | >30000 | >30000 |
| 52 | 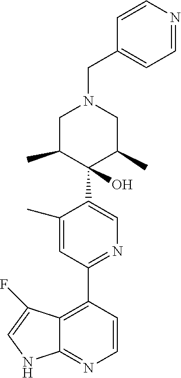 [SMILES: Cc1cc(-c2ccnc3[nH]cc(F)c23)ncc1[C@@]1(O)[C@H](C)CN(Cc2ccncc2)C[C@@H]1C] | >10000 | 2206 | 5084 | | |
| 53 | 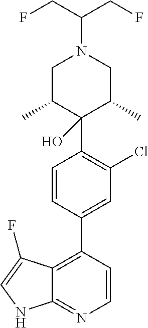 [SMILES: C[C@@H]1CN(C(CF)CF)C[C@H](C)C1(O)c1ccc(-c2ccnc3[nH]cc(F)c23)cc1Cl] | 374 | 3.0 | 3.6 | | |
| 54 | 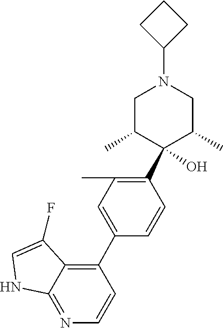 [SMILES: Cc1cc(-c2ccnc3[nH]cc(F)c23)ccc1[C@@]1(O)[C@H](C)CN(C2CCC2)C[C@@H]1C] | 51 | 2.5 | 2.3 | 2970 | 87 |
| 55 | 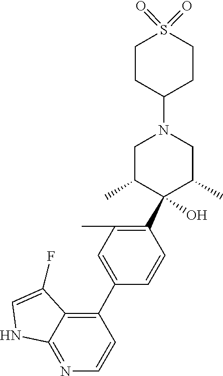 [SMILES: Cc1cc(-c2ccnc3[nH]cc(F)c23)ccc1[C@@]1(O)[C@H](C)CN(C2CCS(=O)(=O)CC2)C[C@@H]1C] | 62.8 | 9.0 | 12.1 | 19800 | 367 |
| 56 | 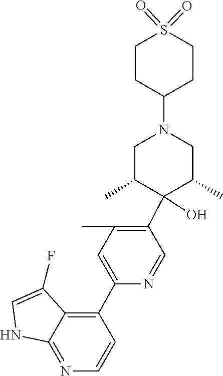 [SMILES: Cc1cc(-c2ccnc3[nH]cc(F)c23)ncc1C1(O)[C@H](C)CN(C2CCS(=O)(=O)CC2)C[C@@H]1C] | >10000 | 2076 | 4246 | | |
| 57 | 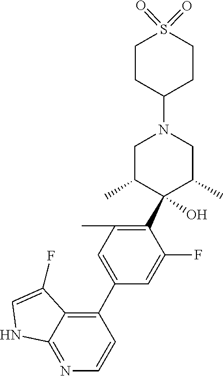 [SMILES: Cc1cc(-c2ccnc3[nH]cc(F)c23)cc(F)c1[C@@]1(O)[C@H](C)CN(C2CCS(=O)(=O)CC2)C[C@@H]1C] | 374 | 4.4 | 5.3 | 31900 | 36.9 |
| 58 | 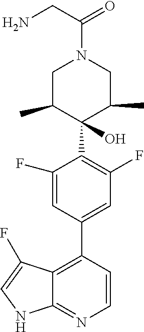 [SMILES: C[C@@H]1CN(C(=O)CN)C[C@H](C)[C@@]1(O)c1c(F)cc(-c2ccnc3[nH]cc(F)c23)cc1F] | 621 | 22.0 | 11.1 | 18200 | 147 |
| 59 | 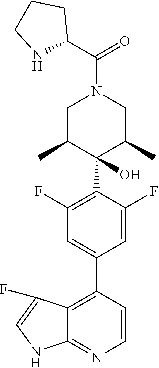 [SMILES: C[C@@H]1CN(C(=O)[C@H]2CCCN2)C[C@H](C)[C@@]1(O)c1c(F)cc(-c2ccnc3[nH]cc(F)c23)cc1F] | 1470 | 21.6 | 10.3 | | |
| 60 | 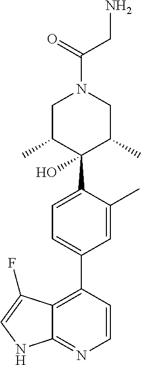 [SMILES: Cc1cc(-c2ccnc3[nH]cc(F)c23)ccc1[C@@]1(O)[C@H](C)CN(C(=O)CN)C[C@@H]1C] | 49.2 | 8.3 | 6.7 | 9350 | 21.5 |
| 61 | 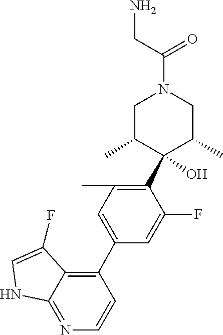 [SMILES: Cc1cc(-c2ccnc3[nH]cc(F)c23)cc(F)c1[C@@]1(O)[C@H](C)CN(C(=O)CN)C[C@@H]1C] | 197 | 3.2 | 3.2 | 3440 | 4.0 |
| 62 | 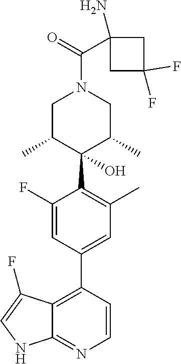 [SMILES: Cc1cc(-c2ccnc3[nH]cc(F)c23)cc(F)c1[C@@]1(O)[C@H](C)CN(C(=O)C2(N)CC(F)(F)C2)C[C@@H]1C] | 5885 | 19.7 | 4.3 | | |
| 63 | 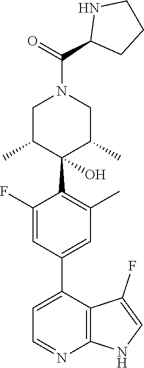 [SMILES: Cc1cc(-c2ccnc3[nH]cc(F)c23)cc(F)c1[C@@]1(O)[C@H](C)CN(C(=O)[C@@H]2CCCN2)C[C@@H]1C] | 677 | 8.5 | 3.4 | | |
| 64 | 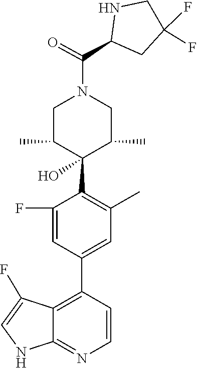 [SMILES: Cc1cc(-c2ccnc3[nH]cc(F)c23)cc(F)c1[C@@]1(O)[C@H](C)CN(C(=O)[C@@H]2CC(F)(F)CN2)C[C@@H]1C] | 3143 | 6.7 | 3.6 | | |
| 65 | 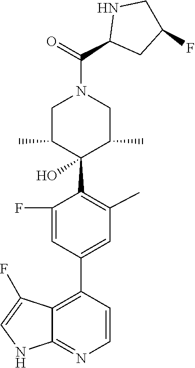 [SMILES: Cc1cc(-c2ccnc3[nH]cc(F)c23)cc(F)c1[C@@]1(O)[C@H](C)CN(C(=O)[C@@H]2C[C@H](F)CN2)C[C@@H]1C] | 1179 | 8.7 | 3.2 | | |
| 66 | 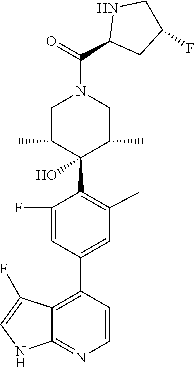 [SMILES: Cc1cc(-c2ccnc3[nH]cc(F)c23)cc(F)c1[C@@]1(O)[C@H](C)CN(C(=O)[C@@H]2C[C@@H](F)CN2)C[C@@H]1C] | 673 | 13.0 | 3.0 | | |
| 67 | 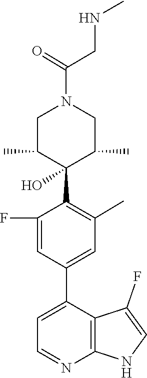 [SMILES: CNCC(=O)N1C[C@@H](C)[C@](O)(c2c(C)cc(-c3ccnc4[nH]cc(F)c34)cc2F)[C@@H](C)C1] | 577 | 8.7 | 2.8 | | |
| 68 | 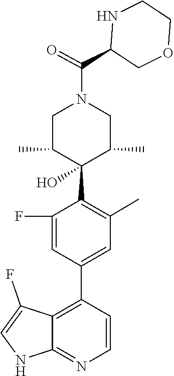 [SMILES: Cc1cc(-c2ccnc3[nH]cc(F)c23)cc(F)c1[C@@]1(O)[C@H](C)CN(C(=O)[C@@H]2COCCN2)C[C@@H]1C] | 1669 | 13.8 | 6.6 | | |
| 69 | 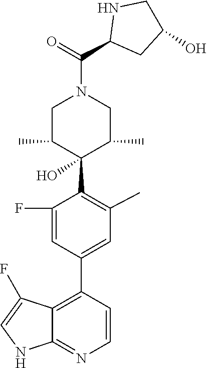 [SMILES: Cc1cc(-c2ccnc3[nH]cc(F)c23)cc(F)c1[C@@]1(O)[C@H](C)CN(C(=O)[C@@H]2C[C@@H](O)CN2)C[C@@H]1C] | 677 | 8.5 | 3.4 | | |
| 70 | 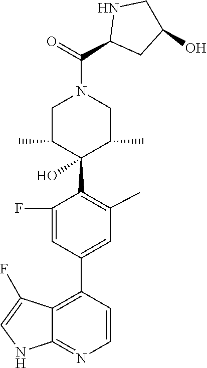 [SMILES: Cc1cc(-c2ccnc3[nH]cc(F)c23)cc(F)c1[C@@]1(O)[C@H](C)CN(C(=O)[C@@H]2C[C@H](O)CN2)C[C@@H]1C] | 376 | 7.8 | 2.5 | | |
| 71 | 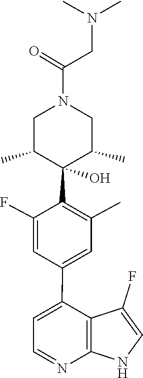 [SMILES: Cc1cc(-c2ccnc3[nH]cc(F)c23)cc(F)c1[C@@]1(O)[C@H](C)CN(C(=O)CN(C)C)C[C@@H]1C] | 939 | 11.3 | 3.7 | | |
| 72 | 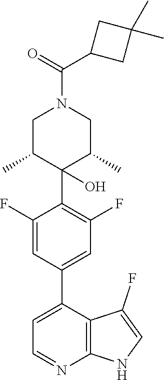 [SMILES: C[C@@H]1CN(C(=O)C2CC(C)(C)C2)C[C@H](C)C1(O)c1c(F)cc(-c2ccnc3[nH]cc(F)c23)cc1F] | >10000 | 27.4 | 60.0 | | |
| 73 | 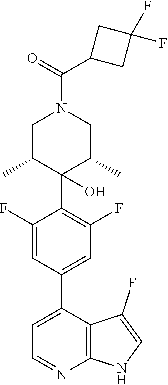 [SMILES: C[C@@H]1CN(C(=O)C2CC(F)(F)C2)C[C@H](C)C1(O)c1c(F)cc(-c2ccnc3[nH]cc(F)c23)cc1F] | 6470 | 14.7 | 32.7 | | |
| 74 | 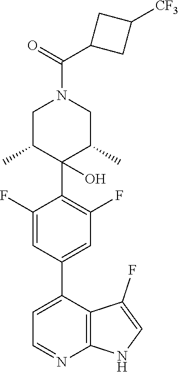 [SMILES: C[C@@H]1CN(C(=O)C2CC(C(F)(F)F)C2)C[C@H](C)C1(O)c1c(F)cc(-c2ccnc3[nH]cc(F)c23)cc1F] | >10000 | 35.3 | 12.2 | | |
| 75 | 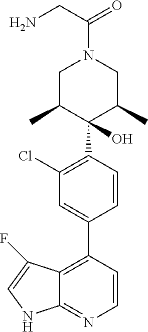 [SMILES: C[C@@H]1CN(C(=O)CN)C[C@H](C)[C@@]1(O)c1ccc(-c2ccnc3[nH]cc(F)c23)cc1Cl] | 140 | 19.9 | 5.0 | | |
| 76 | 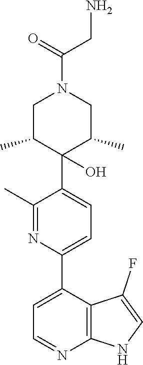 [SMILES: Cc1nc(-c2ccnc3[nH]cc(F)c23)ccc1C1(O)[C@H](C)CN(C(=O)CN)C[C@@H]1C] | 1715 | 29.8 | 18.9 | | |
| 77 | 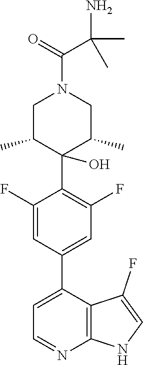 [SMILES: C[C@@H]1CN(C(=O)C(C)(C)N)C[C@H](C)C1(O)c1c(F)cc(-c2ccnc3[nH]cc(F)c23)cc1F] | 885 | 24.6 | 19.3 | | |
| 78 | 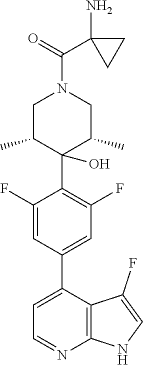 [SMILES: C[C@@H]1CN(C(=O)C2(N)CC2)C[C@H](C)C1(O)c1c(F)cc(-c2ccnc3[nH]cc(F)c23)cc1F] | 2430 | 16.9 | 34.0 | | |
| 79 | 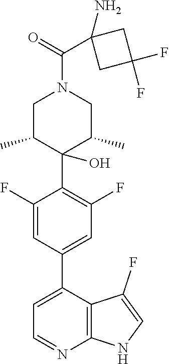 [SMILES: C[C@@H]1CN(C(=O)C2(N)CC(F)(F)C2)C[C@H](C)C1(O)c1c(F)cc(-c2ccnc3[nH]cc(F)c23)cc1F] | 5140 | 12.8 | 29.5 | | |
| 80 | 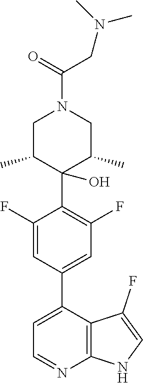 [SMILES: C[C@@H]1CN(C(=O)CN(C)C)C[C@H](C)C1(O)c1c(F)cc(-c2ccnc3[nH]cc(F)c23)cc1F] | 700 | 12.3 | 12.8 | | |
| 81 | 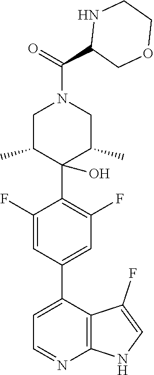 | 1020 | 24.1 | 22.4 | | |
| 82 | 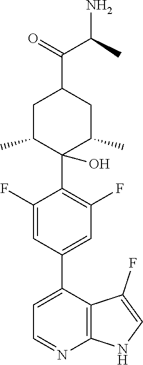 [SMILES: C[C@H](N)C(=O)C1C[C@@H](C)C(O)(c2c(F)cc(-c3ccnc4[nH]cc(F)c34)cc2F)[C@@H](C)C1] | 506 | 14.5 | 14.2 | | |
| 83 | 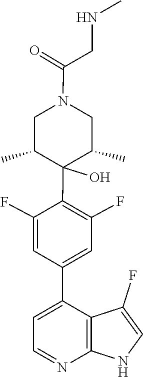 [SMILES: CNCC(=O)N1C[C@@H](C)C(O)(c2c(F)cc(-c3ccnc4[nH]cc(F)c34)cc2F)[C@@H](C)C1] | 570 | 25.0 | 21.3 | | |
| 84 | 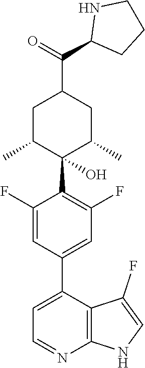 [SMILES: C[C@@H]1CC(C(=O)[C@@H]2CCCN2)C[C@H](C)[C@@]1(O)c1c(F)cc(-c2ccnc3[nH]cc(F)c23)cc1F] | 1470 | 21.6 | 10.3 | | |
| 85 | 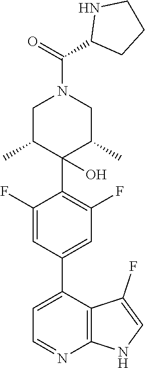 | 659 | 5.8 | 4.7 | | |
| 86 | 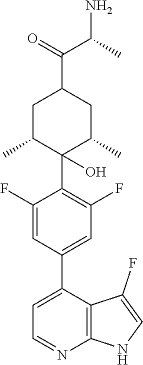 [SMILES: C[C@@H]1CC(C(=O)[C@@H](C)N)C[C@H](C)C1(O)c1c(F)cc(-c2ccnc3[nH]cc(F)c23)cc1F] | 691 | 20.3 | 6.5 | | |
| 87 | 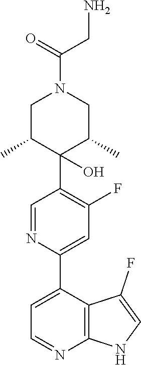 [SMILES: C[C@@H]1CN(C(=O)CN)C[C@H](C)C1(O)c1cnc(-c2ccnc3[nH]cc(F)c23)cc1F] | 4015 | 48.0 | 47.3 | | |
| 88 | 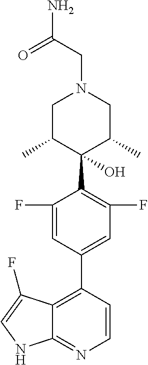 [SMILES: C[C@@H]1CN(CC(N)=O)C[C@H](C)[C@@]1(O)c1c(F)cc(-c2ccnc3[nH]cc(F)c23)cc1F] | 323 | 2.5 | 2.9 | >30000 | 205 |
| 89 | 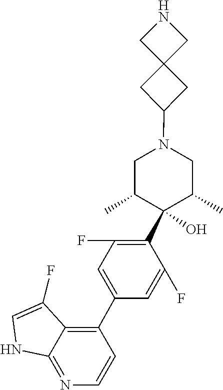 [SMILES: C[C@@H]1CN(C2CC3(CNC3)C2)C[C@H](C)[C@@]1(O)c1c(F)cc(-c2ccnc3[nH]cc(F)c23)cc1F] | 68 | 3.7 | 3.8 | | |
| 90 | 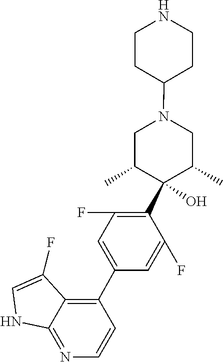 [SMILES: C[C@@H]1CN(C2CCNCC2)C[C@H](C)[C@@]1(O)c1c(F)cc(-c2ccnc3[nH]cc(F)c23)cc1F] | 100 | 4.9 | 4.6 | 32300 | 338 |
| 91 | 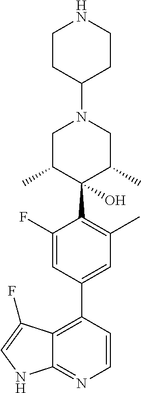 [SMILES: Cc1cc(-c2ccnc3[nH]cc(F)c23)cc(F)c1[C@@]1(O)[C@H](C)CN(C2CCNCC2)C[C@@H]1C] | 179 | 7.9 | 4.2 | | |
| 92 | 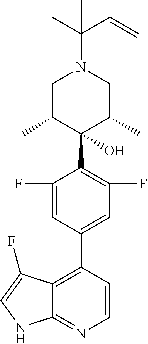 [SMILES: C=CC(C)(C)N1C[C@@H](C)[C@](O)(c2c(F)cc(-c3ccnc4[nH]cc(F)c34)cc2F)[C@@H](C)C1] | | | | | |
| 93 | 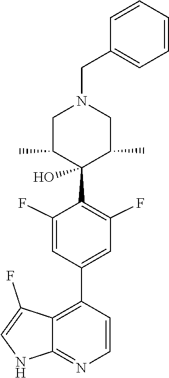 [SMILES: C[C@@H]1CN(Cc2ccccc2)C[C@H](C)[C@@]1(O)c1c(F)cc(-c2ccnc3[nH]cc(F)c23)cc1F] | 2090 | 10.6 | 7.7 | | |
| 94 | 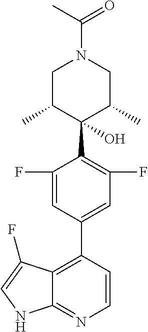 [SMILES: CC(=O)N1C[C@@H](C)[C@](O)(c2c(F)cc(-c3ccnc4[nH]cc(F)c34)cc2F)[C@@H](C)C1] | 4750 | 17.8 | 29.4 | | |
| 95 | 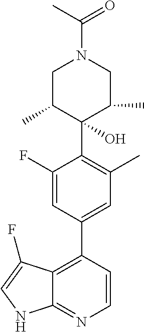 [SMILES: CC(=O)N1C[C@@H](C)[C@](O)(c2c(C)cc(-c3ccnc4[nH]cc(F)c34)cc2F)[C@@H](C)C1] | 2616 | 6.9 | 4.8 | | |
| 96 | 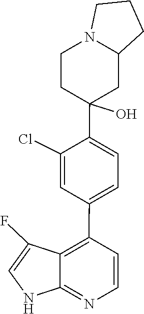 [SMILES: OC1(c2ccc(-c3ccnc4[nH]cc(F)c34)cc2Cl)CCN2CCCC2C1] | 771 | 17.0 | 17.9 | | |
| 97 | 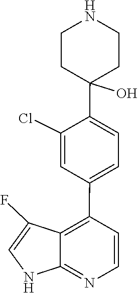 [SMILES: OC1(c2ccc(-c3ccnc4[nH]cc(F)c34)cc2Cl)CCNCC1] | 712 | 11.4 | 42.8 | | |
| 98 | 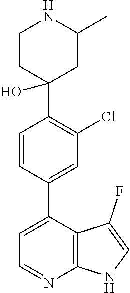 [SMILES: CC1CC(O)(c2ccc(-c3ccnc4[nH]cc(F)c34)cc2Cl)CCN1] | 383 | 24 | 37 | | |
| 99 | 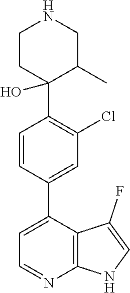 [SMILES: CC1CNCCC1(O)c1ccc(-c2ccnc3[nH]cc(F)c23)cc1Cl] | 219 | 10.6 | 6.2 | | |
| 100 | 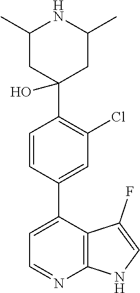 [SMILES: CC1CC(O)(c2ccc(-c3ccnc4[nH]cc(F)c34)cc2Cl)CC(C)N1] | 1900 | 230 | 195 | | |
| 101 | 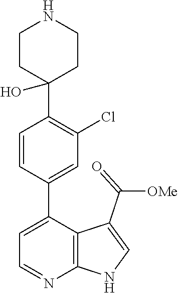 [SMILES: COC(=O)c1c[nH]c2nccc(-c3ccc(C4(O)CCNCC4)c(Cl)c3)c12] | 1952 | 7.1 | 70.8 | | |
| 102 | 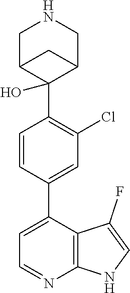 [SMILES: OC1(c2ccc(-c3ccnc4[nH]cc(F)c34)cc2Cl)C2CNCC1C2] | 4872 | 82 | 224 | | |
| 103 | 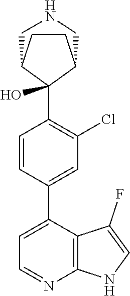 [SMILES: O[C@]1(c2ccc(-c3ccnc4[nH]cc(F)c34)cc2Cl)[C@@H]2CC[C@H]1CNC2] | 1724 | 43.9 | 162 | | |
| 104 | 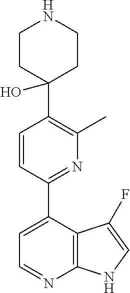 [SMILES: Cc1nc(-c2ccnc3[nH]cc(F)c23)ccc1C1(O)CCNCC1] | 6584 | 145 | 326 | | |
| 105 | 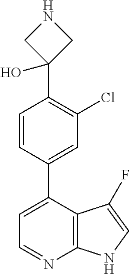 [SMILES: OC1(c2ccc(-c3ccnc4[nH]cc(F)c34)cc2Cl)CNC1] | 828 | 16 | 14 | | |
| 106 | 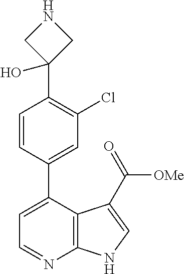 [SMILES: COC(=O)c1c[nH]c2nccc(-c3ccc(C4(O)CNC4)c(Cl)c3)c12] | 885 | 18.2 | 110 | | |
| 107 | 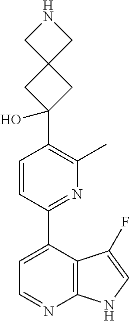 [SMILES: Cc1nc(-c2ccnc3[nH]cc(F)c23)ccc1C1(O)CC2(CNC2)C1] | >10000 | 473 | 661 | | |
| 108 | 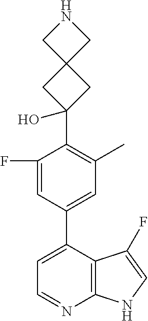 [SMILES: Cc1cc(-c2ccnc3[nH]cc(F)c23)cc(F)c1C1(O)CC2(CNC2)C1] | 1270 | 17.4 | 24.8 | | |
| 109 | 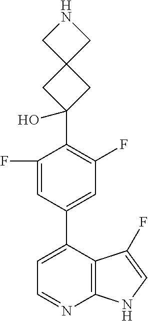 [SMILES: OC1(c2c(F)cc(-c3ccnc4[nH]cc(F)c34)cc2F)CC2(CNC2)C1] | 2980 | 46 | 61 | | |
| 110 | 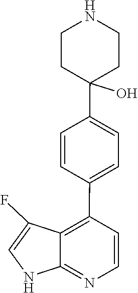 [SMILES: OC1(c2ccc(-c3ccnc4[nH]cc(F)c34)cc2)CCNCC1] | 490 | 5.7 | 2.9 | 13300 | 17700 |
| 111 | 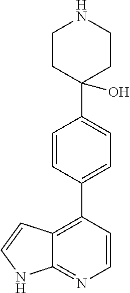 [SMILES: OC1(c2ccc(-c3ccnc4[nH]ccc34)cc2)CCNCC1] | 1098 | 22 | 22 | >50000 | >50000 |
| 112 | 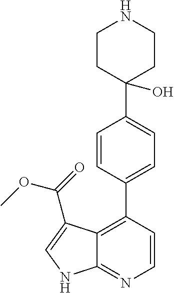 [SMILES: COC(=O)c1c[nH]c2nccc(-c3ccc(C4(O)CCNCC4)cc3)c12] | 2330 | 9.2 | 18 | | |
| 113 | 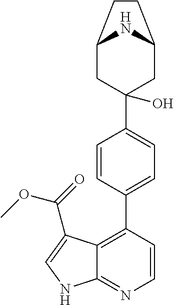 [SMILES: COC(=O)c1c[nH]c2nccc(-c3ccc(C4(O)C[C@H]5CC[C@@H](C4)N5)cc3)c12] | 1064 | 12.4 | 65.5 | | |
| 114 | 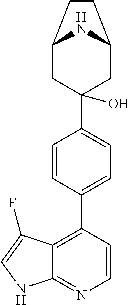 [SMILES: OC1(c2ccc(-c3ccnc4[nH]cc(F)c34)cc2)C[C@H]2CC[C@@H](C1)N2] | 390 | 23 | 19 | | |
| 115 | 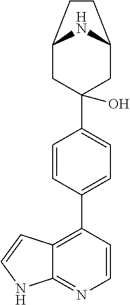 [SMILES: OC1(c2ccc(-c3ccnc4[nH]ccc34)cc2)C[C@H]2CC[C@@H](C1)N2] | 176 | 7.2 | 10 | | |
| 116 | 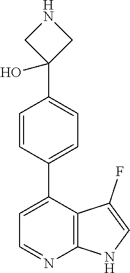 [SMILES: OC1(c2ccc(-c3ccnc4[nH]cc(F)c34)cc2)CNC1] | 468 | 9.2 | 16.7 | | |
| 117 | 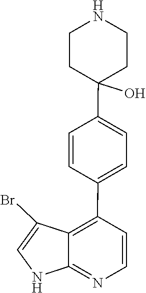 [SMILES: OC1(c2ccc(-c3ccnc4[nH]cc(Br)c34)cc2)CCNCC1] | 240 | 4.2 | 3.2 | | |
| 118 | 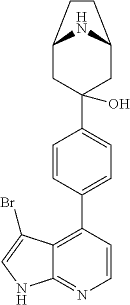 [SMILES: OC1(c2ccc(-c3ccnc4[nH]cc(Br)c34)cc2)C[C@H]2CC[C@@H](C1)N2] | 14 | 1.8 | 0.9 | | |
| 119 | 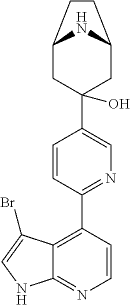 [SMILES: OC1(c2ccc(-c3ccnc4[nH]cc(Br)c34)nc2)C[C@H]2CC[C@@H](C1)N2] | 2593 | 29.8 | 184.5 | | |
| 120 | 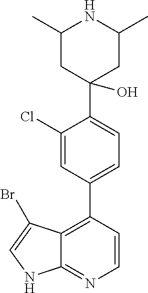 [SMILES: CC1CC(O)(c2ccc(-c3ccnc4[nH]cc(Br)c34)cc2Cl)CC(C)N1] | 103 | 58 | 30 | | |
| 121 | 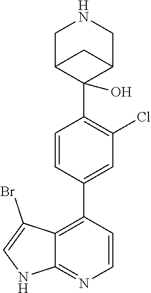 [SMILES: OC1(c2ccc(-c3ccnc4[nH]cc(Br)c34)cc2Cl)C2CNCC1C2] | 307 | 10.2 | 14.6 | | |
| 122 | 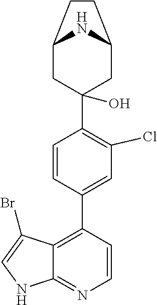 [SMILES: OC1(c2ccc(-c3ccnc4[nH]cc(Br)c34)cc2Cl)C[C@H]2CC[C@@H](C1)N2] | 51.2 | 5.3 | 8.9 | | |
| 123 | 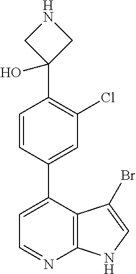 [SMILES: OC1(c2ccc(-c3ccnc4[nH]cc(Br)c34)cc2Cl)CNC1] | 158 | 12 | 7 | | |
| 124 | 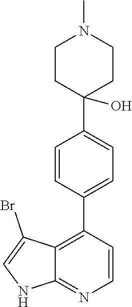 [SMILES: CN1CCC(O)(c2ccc(-c3ccnc4[nH]cc(Br)c34)cc2)CC1] | 128 | 5.7 | 3.7 | | |
| 125 | 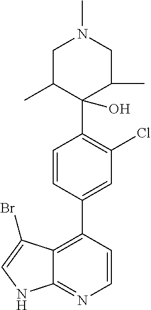 [SMILES: CC1CN(C)CC(C)C1(O)c1ccc(-c2ccnc3[nH]cc(Br)c23)cc1Cl] | 108 | 14.1 | 7.0 | | |
| 126 | 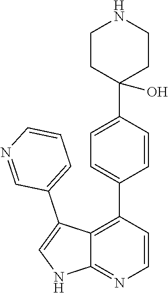 [SMILES: OC1(c2ccc(-c3ccnc4[nH]cc(-c5cccnc5)c34)cc2)CCNCC1] | 245 | 9.1 | 32 | | |
| 127 | 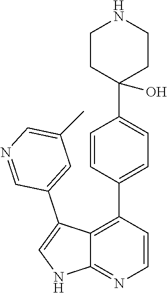 [SMILES: Cc1cncc(-c2c[nH]c3nccc(-c4ccc(C5(O)CCNCC5)cc4)c23)c1] | 4300 | 19 | 40 | | |
| 128 | 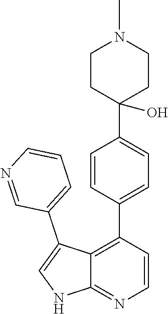 [SMILES: CN1CCC(O)(c2ccc(-c3ccnc4[nH]cc(-c5cccnc5)c34)cc2)CC1] | 2050 | 31 | 36 | | |
| 129 | 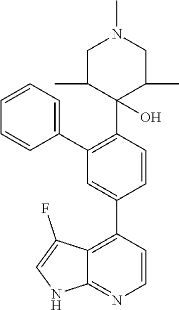 [SMILES: CC1CN(C)CC(C)C1(O)c1ccc(-c2ccnc3[nH]cc(F)c23)cc1-c1ccccc1] | 233 | 157 | 190 | | |
| 130 | 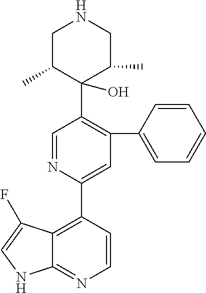 [SMILES: C[C@@H]1CNC[C@H](C)C1(O)c1cnc(-c2ccnc3[nH]cc(F)c23)cc1-c1ccccc1] | 2264 | 146 | 381 | | |
| 131 | 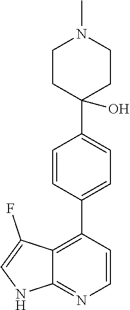 [SMILES: CN1CCC(O)(c2ccc(-c3ccnc4[nH]cc(F)c34)cc2)CC1] | 1070 | 28 | 25 | | |
| 132 | 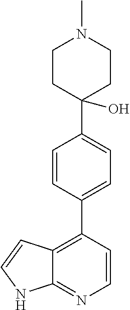 [SMILES: CN1CCC(O)(c2ccc(-c3ccnc4[nH]ccc34)cc2)CC1] | 490 | 2.9 | 5.7 | | |
| 133 | 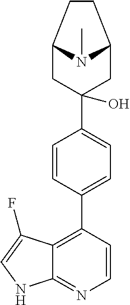 [SMILES: CN1[C@@H]2CC[C@H]1CC(O)(c1ccc(-c3ccnc4[nH]cc(F)c34)cc1)C2] | 389.8 | 20.1 | 19.1 | | |
| 134 | 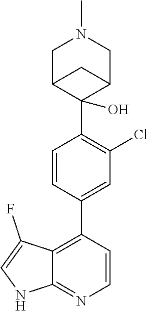 [SMILES: CN1CC2CC(C1)C2(O)c1ccc(-c2ccnc3[nH]cc(F)c23)cc1Cl] | 788 | 43.5 | 116 | | |
| 135 | 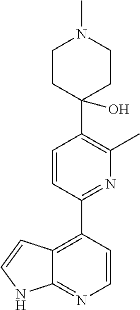 [SMILES: Cc1nc(-c2ccnc3[nH]ccc23)ccc1C1(O)CCN(C)CC1] | 7959 | 736 | 682 | | |
| 136 | 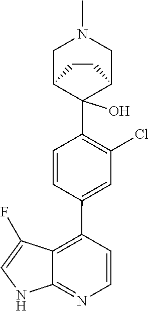 [SMILES: CN1C[C@H]2CC[C@@H](C1)C2(O)c1ccc(-c2ccnc3[nH]cc(F)c23)cc1Cl] | 1063 | 69.8 | 118 | | |
| 137 | 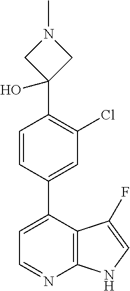 [SMILES: CN1CC(O)(c2ccc(-c3ccnc4[nH]cc(F)c34)cc2Cl)C1] | 1178 | 108 | 350 | | |
| 138 | 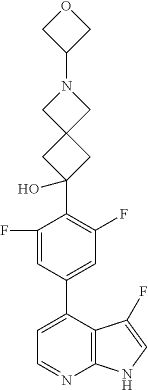 [SMILES: OC1(c2c(F)cc(-c3ccnc4[nH]cc(F)c34)cc2F)CC2(CN(C3COC3)C2)C1] | 2710 | 66.4 | 117 | | |
| 139 | 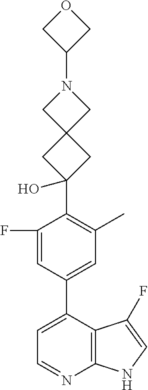 [SMILES: Cc1cc(-c2ccnc3[nH]cc(F)c23)cc(F)c1C1(O)CC2(CN(C3COC3)C2)C1] | 698 | 5.9 | 9.0 | >30000 | 58.2 |
| 140 | 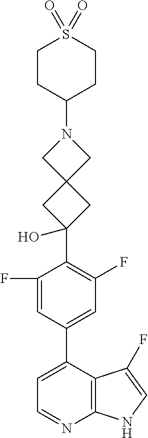 [SMILES: O=S1(=O)CCC(N2CC3(C2)CC(O)(c2c(F)cc(-c4ccnc5[nH]cc(F)c45)cc2F)C3)CC1] | 1580 | 56.8 | 120 | | |
| 141 | 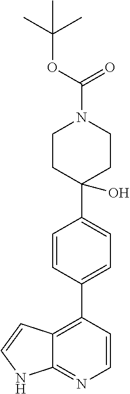 [SMILES: CC(C)(C)OC(=O)N1CCC(O)(c2ccc(-c3ccnc4[nH]ccc34)cc2)CC1] | >10000 | 896.7 | 464.8 | | |
| 142 | 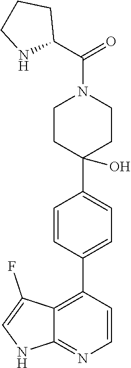 [SMILES: O=C([C@H]1CCCN1)N1CCC(O)(c2ccc(-c3ccnc4[nH]cc(F)c34)cc2)CC1] | 1350 | 7.8 | 4.4 | 39800 | 1840 |
| 143 | 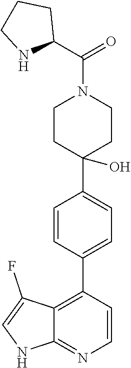 [SMILES: O=C([C@@H]1CCCN1)N1CCC(O)(c2ccc(-c3ccnc4[nH]cc(F)c34)cc2)CC1] | 1460 | 26 | 12 | | |
| 144 | 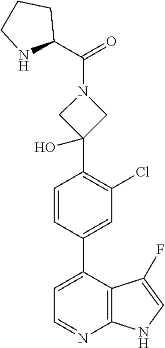 [SMILES: O=C([C@@H]1CCCN1)N1CC(O)(c2ccc(-c3ccnc4[nH]cc(F)c34)cc2Cl)C1] | 2770 | 50 | 26 | | |
| 145 | 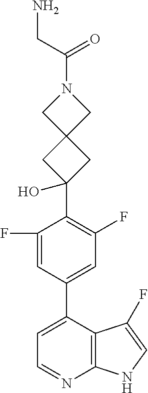 [SMILES: NCC(=O)N1CC2(C1)CC(O)(c1c(F)cc(-c3ccnc4[nH]cc(F)c34)cc1F)C2] | 1870 | 28.3 | 46.0 | | |
| 146 | 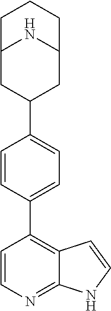 [SMILES: c1cc(-c2ccc(C3CC4CCCC(C3)N4)cc2)c2cc[nH]c2n1] | 2460 | 202.6 | 94.8 | | |
| 147 | 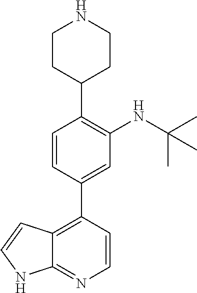 [SMILES: CC(C)(C)Nc1cc(-c2ccnc3[nH]ccc23)ccc1C1CCNCC1] | 164.5 | 216.2 | 108.5 | | |
| 148 | 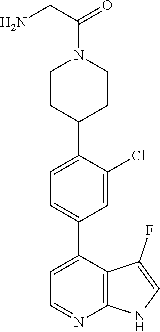 [SMILES: NCC(=O)N1CCC(c2ccc(-c3ccnc4[nH]cc(F)c34)cc2Cl)CC1] | 1084 | 7.7 | 17.8 | | |
| 149 | 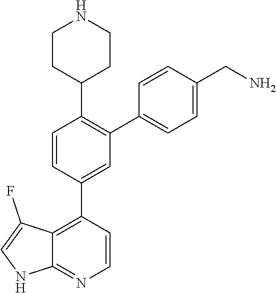 [SMILES: NCc1ccc(-c2cc(-c3ccnc4[nH]cc(F)c34)ccc2C2CCNCC2)cc1] | 67.3 | 4.3 | 1.0 | | |
| 150 | 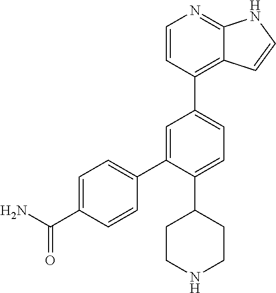 [SMILES: NC(=O)c1ccc(-c2cc(-c3ccnc4[nH]ccc34)ccc2C2CCNCC2)cc1] | 967 | 32.9 | 25.9 | | |
| 151 | 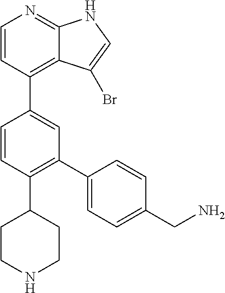 [SMILES: NCc1ccc(-c2cc(-c3ccnc4[nH]cc(Br)c34)ccc2C2CCNCC2)cc1] | 170 | 16.6 | 15.1 | | |
| 152 | 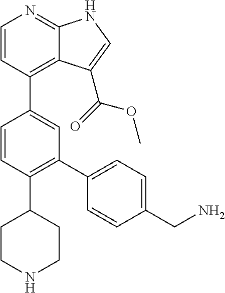 [SMILES: COC(=O)c1c[nH]c2nccc(-c3ccc(C4CCNCC4)c(-c4ccc(CN)cc4)c3)c12] | 4410 | 66.3 | 50.2 | | |
| 153 | 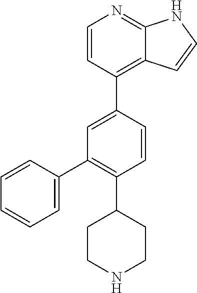 [SMILES: c1ccc(-c2cc(-c3ccnc4[nH]ccc34)ccc2C2CCNCC2)cc1] | 377.9 | 15.2 | 20.4 | | |
| 154 | 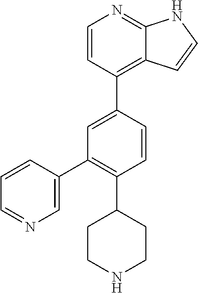 [SMILES: c1cncc(-c2cc(-c3ccnc4[nH]ccc34)ccc2C2CCNCC2)c1] | 1320 | 87 | 48 | | |
| 155 | 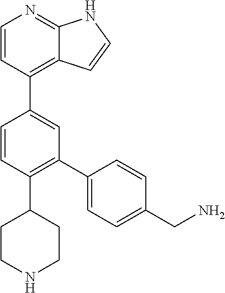 [SMILES: NCc1ccc(-c2cc(-c3ccnc4[nH]ccc34)ccc2C2CCNCC2)cc1] | 68 | 4.3 | 1.0 | | |
| 156 | 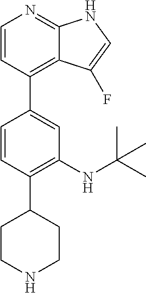 [SMILES: CC(C)(C)Nc1cc(-c2ccnc3[nH]cc(F)c23)ccc1C1CCNCC1] | 1120 | 139 | 121 | | |
| 157 | 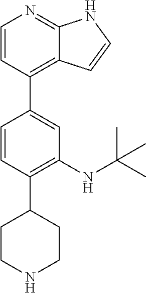 [SMILES: CC(C)(C)Nc1cc(-c2ccnc3[nH]ccc23)ccc1C1CCNCC1] | 1650 | 216 | 109 | | |
| 158 | 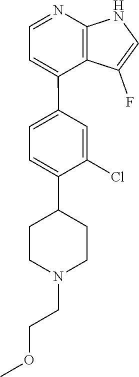 [SMILES: COCCN1CCC(c2ccc(-c3ccnc4[nH]cc(F)c34)cc2Cl)CC1] | 1150 | 29 | 56 | | |
| 159 | 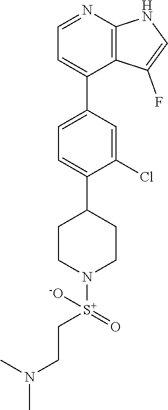 | 8520 | 174 | 139 | | |
| 160 | 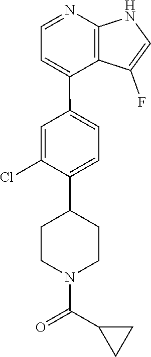 [SMILES: O=C(C1CC1)N1CCC(c2ccc(-c3ccnc4[nH]cc(F)c34)cc2Cl)CC1] | >10000 | 451 | 442 | | |
| 161 | 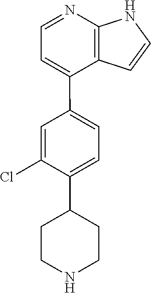 [SMILES: Clc1cc(-c2ccnc3[nH]ccc23)ccc1C1CCNCC1] | 753 | 8.9 | 21 | | |
| 162 | 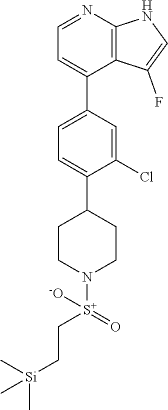 | >10000 | >10000 | >10000 | | |
| 163 | 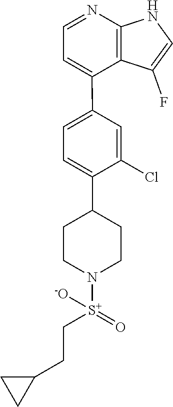 | >10000 | >10000 | >10000 | | |
| 164 | 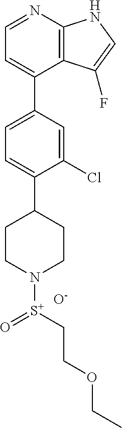 | >10000 | >10000 | >10000 | | |
| 165 | 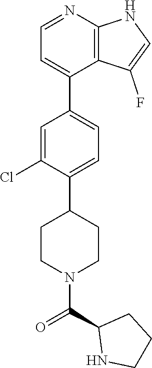 [SMILES: O=C([C@H]1CCCN1)N1CCC(c2ccc(-c3ccnc4[nH]cc(F)c34)cc2Cl)CC1] | 1514 | 32.1 | 36.9 | | |
| 166 | 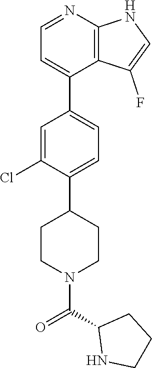 [SMILES: O=C([C@@H]1CCCN1)N1CCC(c2ccc(-c3ccnc4[nH]cc(F)c34)cc2Cl)CC1] | 1302 | 10.7 | 15.5 | | |
| 167 | 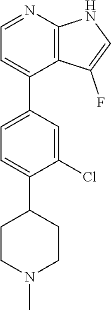 [SMILES: CN1CCC(c2ccc(-c3ccnc4[nH]cc(F)c34)cc2Cl)CC1] | 1520 | 18 | 31 | | |
| 168 | 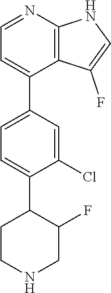 [SMILES: Fc1c[nH]c2nccc(-c3ccc(C4CCNCC4F)c(Cl)c3)c12] | | | | | |
| 169 | 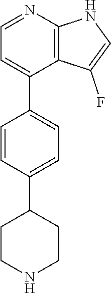 [SMILES: Fc1c[nH]c2nccc(-c3ccc(C4CCNCC4)cc3)c12] | 1180 | 15 | 14 | 13300 | 25800 |
| 170 | 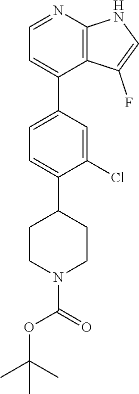 [SMILES: CC(C)(C)OC(=O)N1CCC(c2ccc(-c3ccnc4[nH]cc(F)c34)cc2Cl)CC1] | >10000 | >10000 | >10000 | | |
| 171 | 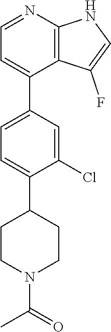 [SMILES: CC(=O)N1CCC(c2ccc(-c3ccnc4[nH]cc(F)c34)cc2Cl)CC1] | 5400 | 69 | 100 | | |
| 172 | 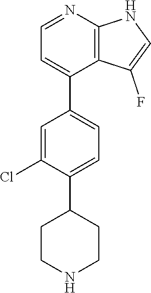 [SMILES: Fc1c[nH]c2nccc(-c3ccc(C4CCNCC4)c(Cl)c3)c12] | 715 | 9.6 | 19 | >50000 | >50000 |
| 173 | 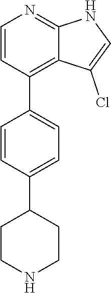 [SMILES: Clc1c[nH]c2nccc(-c3ccc(C4CCNCC4)cc3)c12] | 330 | 7.2 | 8.0 | 73900 | 1200 |
| 174 | 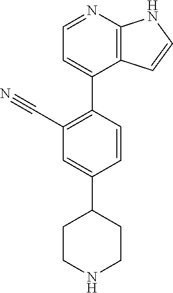 [SMILES: N#Cc1cc(C2CCNCC2)ccc1-c1ccnc2[nH]ccc12] | 9000 | 4930 | 2410 | | |
| 175 | 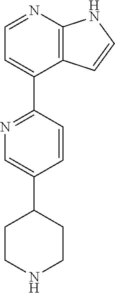 [SMILES: c1cc(-c2ccc(C3CCNCC3)cn2)c2cc[nH]c2n1] | 7000 | 56 | 142 | | |
| 176 | 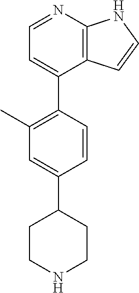 [SMILES: Cc1cc(C2CCNCC2)ccc1-c1ccnc2[nH]ccc12] | >10000 | 1100 | 2140 | | |
| 177 | 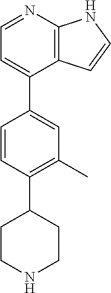 [SMILES: Cc1cc(-c2ccnc3[nH]ccc23)ccc1C1CCNCC1] | 684 | 11 | 14 | 32000 | 48300 |
| 178 | 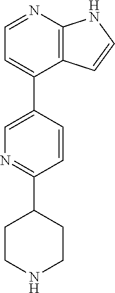 [SMILES: c1cc(-c2ccc(C3CCNCC3)nc2)c2cc[nH]c2n1] | 640 | 63 | 71 | | |
| 179 | 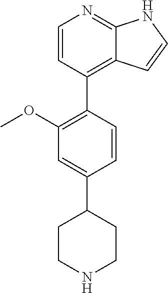 [SMILES: COc1cc(C2CCNCC2)ccc1-c1ccnc2[nH]ccc12] | 5660 | 1080 | 1290 | | |
| 180 | 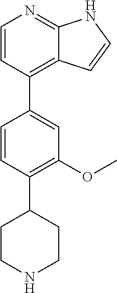 [SMILES: COc1cc(-c2ccnc3[nH]ccc23)ccc1C1CCNCC1] | 2570 | 69 | 84 | | |
| 181 | 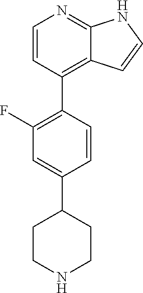 [SMILES: Fc1cc(C2CCNCC2)ccc1-c1ccnc2[nH]ccc12] | 3360 | 68 | 87 | | |
| 182 | 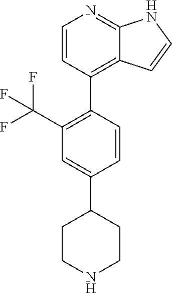 [SMILES: FC(F)(F)c1cc(C2CCNCC2)ccc1-c1ccnc2[nH]ccc12] | >10000 | 1570 | 1770 | | |
| 183 | 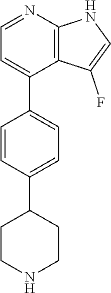 [SMILES: Fc1c[nH]c2nccc(-c3ccc(C4CCNCC4)cc3)c12] | 1180 | 15 | 14 | 13300 | 25800 |
| 184 | 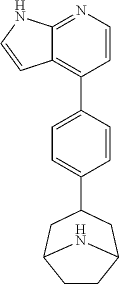 [SMILES: c1cc(-c2ccc(C3CC4CCC(C3)N4)cc2)c2cc[nH]c2n1] | 940 | 25 | 50 | 27400 | 57400 |
| 185 | 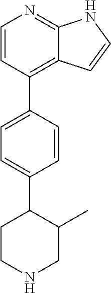 [SMILES: CC1CNCCC1c1ccc(-c2ccnc3[nH]ccc23)cc1] | 2110 | 70 | 57 | | |
| 186 | 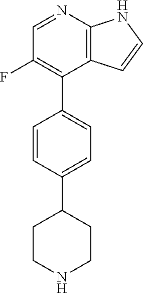 [SMILES: Fc1cnc2[nH]ccc2c1-c1ccc(C2CCNCC2)cc1] | 2050 | 36 | 68 | | |
| 187 | 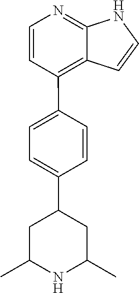 [SMILES: CC1CC(c2ccc(-c3ccnc4[nH]ccc34)cc2)CC(C)N1] | 1441 | 66.1 | 62.6 | >50000 | >50000 |
| 188 | 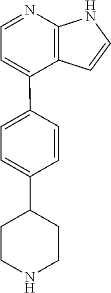 [SMILES: c1cc(-c2ccc(C3CCNCC3)cc2)c2cc[nH]c2n1] | 2200 | 70 | 75 | | |
| 189 | 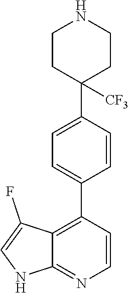 [SMILES: Fc1c[nH]c2nccc(-c3ccc(C4(C(F)(F)F)CCNCC4)cc3)c12] | 18 | 11 | 14 | | |
| 190 | 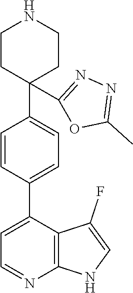 [SMILES: Cc1nnc(C2(c3ccc(-c4ccnc5[nH]cc(F)c45)cc3)CCNCC2)o1] | 18 | 20 | 17 | | |
| 191 | 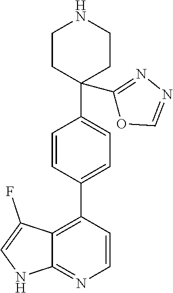 [SMILES: Fc1c[nH]c2nccc(-c3ccc(C4(c5nnco5)CCNCC4)cc3)c12] | 200 | 57 | 43 | | |
| 192 | 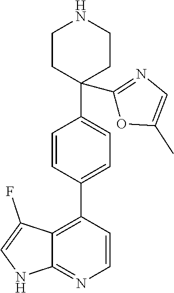 [SMILES: Cc1cnc(C2(c3ccc(-c4ccnc5[nH]cc(F)c45)cc3)CCNCC2)o1] | 25 | 29 | 20 | | |
| 193 | 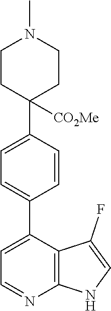 [SMILES: COC(=O)C1(c2ccc(-c3ccnc4[nH]cc(F)c34)cc2)CCN(C)CC1] | 128 | 53 | 26 | | |
| 194 | 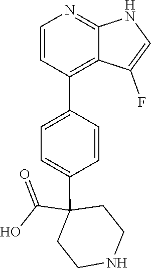 [SMILES: O=C(O)C1(c2ccc(-c3ccnc4[nH]cc(F)c34)cc2)CCNCC1] | >10000 | >10000 | >10000 | | |
| 195 | 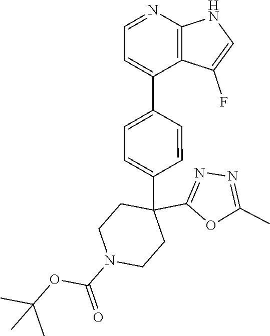 [SMILES: Cc1nnc(C2(c3ccc(-c4ccnc5[nH]cc(F)c45)cc3)CCN(C(=O)OC(C)(C)C)CC2)o1] | >10000 | 5790 | 3520 | | |
| 196 | 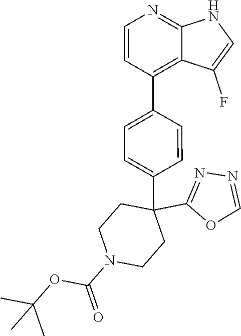 [SMILES: CC(C)(C)OC(=O)N1CCC(c2ccc(-c3ccnc4[nH]cc(F)c34)cc2)(c2nnco2)CC1] | >10000 | >10000 | >10000 | | |
| 197 | 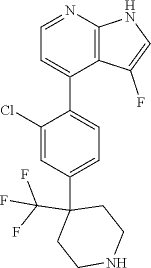 [SMILES: Fc1c[nH]c2nccc(-c3ccc(C4(C(F)(F)F)CCNCC4)cc3Cl)c12] | 85 | 424 | 195 | | |
| 198 | 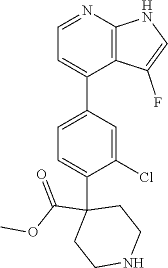 [SMILES: COC(=O)C1(c2ccc(-c3ccnc4[nH]cc(F)c34)cc2Cl)CCNCC1] | 124 | 7.4 | 10 | | |
| 199 | 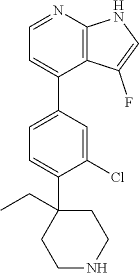 [SMILES: CCC1(c2ccc(-c3ccnc4[nH]cc(F)c34)cc2Cl)CCNCC1] | 463 | 38 | 34 | | |
| 200 | 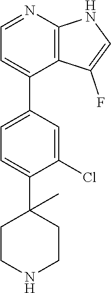 [SMILES: CC1(c2ccc(-c3ccnc4[nH]cc(F)c34)cc2Cl)CCNCC1] | 12 | 34 | 20 | | |
| 201 | 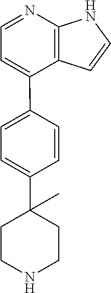 [SMILES: CC1(c2ccc(-c3ccnc4[nH]ccc34)cc2)CCNCC1] | 200 | 59 | 43 | 7510 | >50000 |
| 202 | 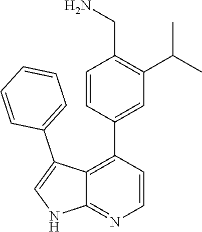 [SMILES: CC(C)c1cc(-c2ccnc3[nH]cc(-c4ccccc4)c23)ccc1CN] | 743 | 17 | 7 | | |
| 203 | 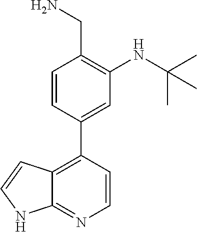 [SMILES: CC(C)(C)Nc1cc(-c2ccnc3[nH]ccc23)ccc1CN] | 1382 | 70.3 | 135 | | |
| 204 | 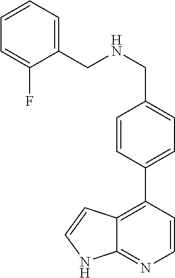 [SMILES: Fc1ccccc1CNCc1ccc(-c2ccnc3[nH]ccc23)cc1] | 1434 | 15.9 | 33.1 | | |
| 205 | 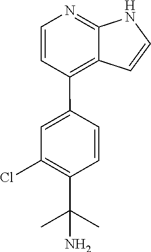 [SMILES: CC(C)(N)c1ccc(-c2ccnc3[nH]ccc23)cc1Cl] | 437 | 9.5 | 31.8 | | |
| 206 | 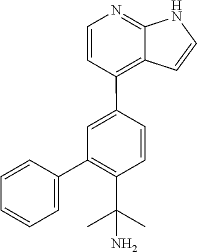 [SMILES: CC(C)(N)c1ccc(-c2ccnc3[nH]ccc23)cc1-c1ccccc1] | 1624 | 10.2 | 74.9 | | |
| 207 | 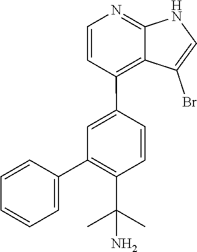 [SMILES: CC(C)(N)c1ccc(-c2ccnc3[nH]cc(Br)c23)cc1-c1ccccc1] | 267 | 4.0 | 9.6 | | |
| 208 | 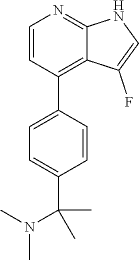 [SMILES: CN(C)C(C)(C)c1ccc(-c2ccnc3[nH]cc(F)c23)cc1] | 349 | 8.3 | 15 | | |
| 209 | 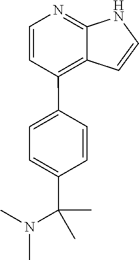 [SMILES: CN(C)C(C)(C)c1ccc(-c2ccnc3[nH]ccc23)cc1] | 1250 | 43 | 146 | | |
| 210 | 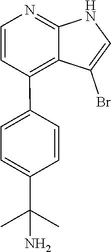 [SMILES: CC(C)(N)c1ccc(-c2ccnc3[nH]cc(Br)c23)cc1] | 331 | 24 | 4.7 | | |
| 211 | 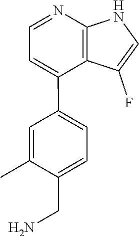 [SMILES: Cc1cc(-c2ccnc3[nH]cc(F)c23)ccc1CN] | 312 | 3.7 | 5.5 | 22600 | 6370 |
| 212 | 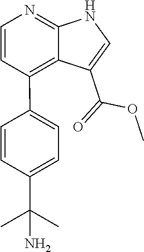 [SMILES: COC(=O)c1c[nH]c2nccc(-c3ccc(C(C)(C)N)cc3)c12] | 1250 | 12 | 13 | | |
| 213 | 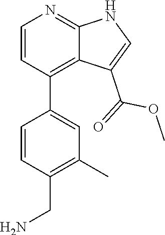 [SMILES: COC(=O)c1c[nH]c2nccc(-c3ccc(CN)c(C)c3)c12] | 832 | 7.7 | 14.7 | | |
| 214 | 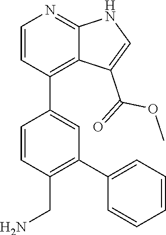 [SMILES: COC(=O)c1c[nH]c2nccc(-c3ccc(CN)c(-c4ccccc4)c3)c12] | 986 | 11 | 24 | | |
| 215 | 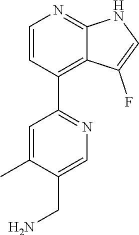 [SMILES: Cc1cc(-c2ccnc3[nH]cc(F)c23)ncc1CN] | 2840 | 4.7 | 15 | | |
| 216 | 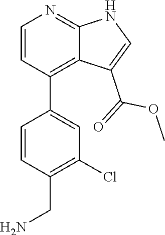 [SMILES: COC(=O)c1c[nH]c2nccc(-c3ccc(CN)c(Cl)c3)c12] | 823 | 11 | 19 | | |
| 217 | 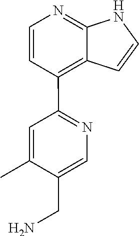 [SMILES: Cc1cc(-c2ccnc3[nH]ccc23)ncc1CN] | 3160 | 8.2 | 12 | | |
| 218 | 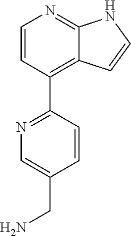 [SMILES: NCc1ccc(-c2ccnc3[nH]ccc23)nc1] | 4930 | 37 | 130 | | |
| 219 | 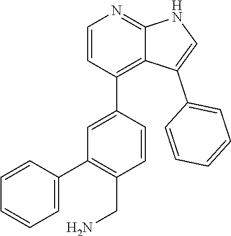 [SMILES: NCc1ccc(-c2ccnc3[nH]cc(-c4ccccc4)c23)cc1-c1ccccc1] | 394 | 39 | 128 | >50000 | 653 |
| 220 | 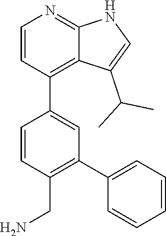 [SMILES: CC(C)c1c[nH]c2nccc(-c3ccc(CN)c(-c4ccccc4)c3)c12] | 1010 | 236 | 115 | | |
| 221 | 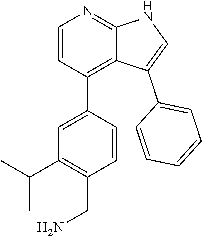 [SMILES: CC(C)c1cc(-c2ccnc3[nH]cc(-c4ccccc4)c23)ccc1CN] | 743 | 17 | 7 | | |
| 222 | 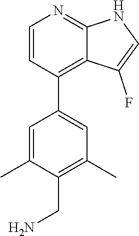 [SMILES: Cc1cc(-c2ccnc3[nH]cc(F)c23)cc(C)c1CN] | 470 | 16 | 31 | | |
| 223 | 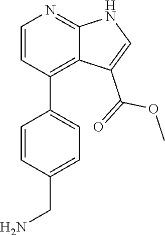 [SMILES: COC(=O)c1c[nH]c2nccc(-c3ccc(CN)cc3)c12] | 13 | 27.4 | 1416 | >50000 | 66800 |
| 224 | 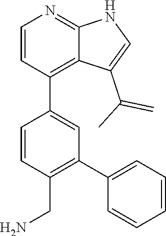 [SMILES: C=C(C)c1c[nH]c2nccc(-c3ccc(CN)c(-c4ccccc4)c3)c12] | 1150 | 73 | 35 | | |
| 225 | 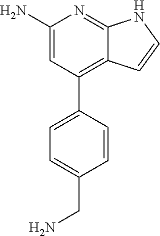 [SMILES: NCc1ccc(-c2cc(N)nc3[nH]ccc23)cc1] | >10000 | 10000 | 6418 | | |
| 226 | 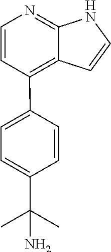 [SMILES: CC(C)(N)c1ccc(-c2ccnc3[nH]ccc23)cc1] | 734 | 25.4 | 1.2 | | |
| 227 | 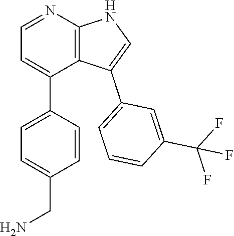 [SMILES: NCc1ccc(-c2ccnc3[nH]cc(-c4cccc(C(F)(F)F)c4)c23)cc1] | 3672 | 124 | 49.3 | | |
| 228 | 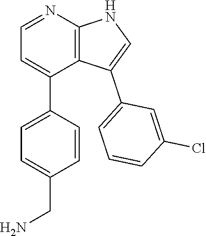 [SMILES: NCc1ccc(-c2ccnc3[nH]cc(-c4cccc(Cl)c4)c23)cc1] | 1015 | 34 | 14 | | |
| 229 | 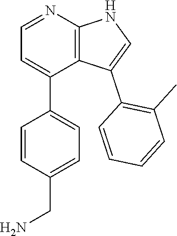 [SMILES: Cc1ccccc1-c1c[nH]c2nccc(-c3ccc(CN)cc3)c12] | 6342 | 235 | 235 | | |
| 230 | 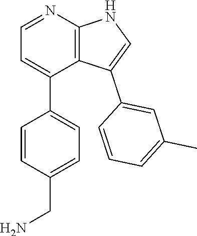 [SMILES: Cc1cccc(-c2c[nH]c3nccc(-c4ccc(CN)cc4)c23)c1] | 1197 | 17 | 16 | 752 | 5470 |
| 231 | 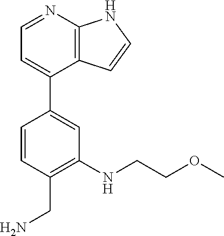 [SMILES: COCCNc1cc(-c2ccnc3[nH]ccc23)ccc1CN] | 437 | 24 | 144 | | |
| 232 | 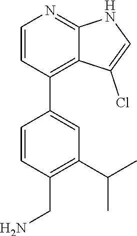 [SMILES: CC(C)c1cc(-c2ccnc3[nH]cc(Cl)c23)ccc1CN] | 60.5 | 3.3 | 11.6 | | |
| 233 | 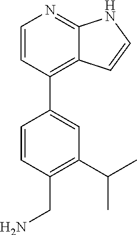 [SMILES: CC(C)c1cc(-c2ccnc3[nH]ccc23)ccc1CN] | 281 | 5.6 | 35.5 | | |
| 234 | 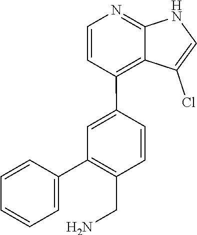 [SMILES: NCc1ccc(-c2ccnc3[nH]cc(Cl)c23)cc1-c1ccccc1] | 68.4 | 6.5 | 23.9 | | |
| 235 | 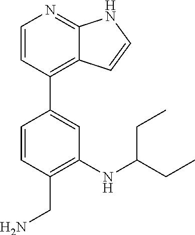 [SMILES: CCC(CC)Nc1cc(-c2ccnc3[nH]ccc23)ccc1CN] | 788 | 412 | 371 | | |
| 236 | 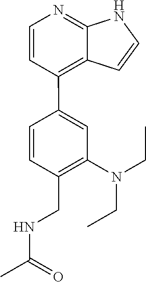 [SMILES: CCN(CC)c1cc(-c2ccnc3[nH]ccc23)ccc1CNC(C)=O] | 3595 | 772 | 887 | | |
| 237 | 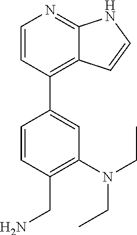 [SMILES: CCN(CC)c1cc(-c2ccnc3[nH]ccc23)ccc1CN] | 100 | 12 | 23 | | |
| 238 | 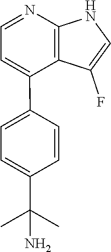 [SMILES: CC(C)(N)c1ccc(-c2ccnc3[nH]cc(F)c23)cc1] | 229 | 1.1 | 1.2 | 9530 | 1560 |
| 239 | 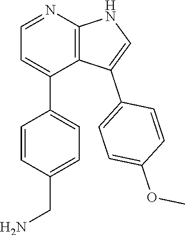 [SMILES: COc1ccc(-c2c[nH]c3nccc(-c4ccc(CN)cc4)c23)cc1] | 1310 | 67 | 26 | | |
| 240 | 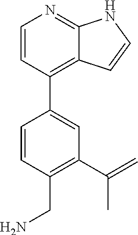 [SMILES: C=C(C)c1cc(-c2ccnc3[nH]ccc23)ccc1CN] | 178 | 4.3 | 33.8 | | |
| 241 | 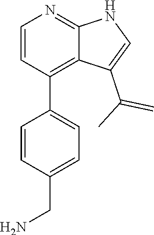 [SMILES: C=C(C)c1c[nH]c2nccc(-c3ccc(CN)cc3)c12] | 809 | 19 | 6 | | |
| 242 | 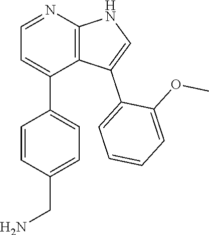 [SMILES: COc1ccccc1-c1c[nH]c2nccc(-c3ccc(CN)cc3)c12] | 1320 | 11 | 10 | | |
| 243 | 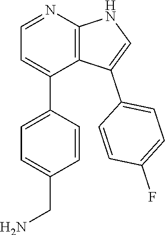 [SMILES: NCc1ccc(-c2ccnc3[nH]cc(-c4ccc(F)cc4)c23)cc1] | 2330 | 72 | 24 | | |
| 244 | 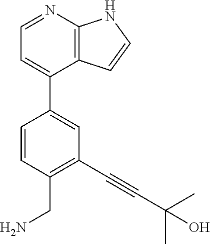 [SMILES: CC(C)(O)C#Cc1cc(-c2ccnc3[nH]ccc23)ccc1CN] | 1250 | 20 | 28 | | |
| 245 | 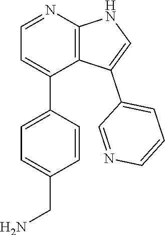 [SMILES: NCc1ccc(-c2ccnc3[nH]cc(-c4cccnc4)c23)cc1] | 751 | 12 | 24 | 3450 | 11900 |
| 246 | 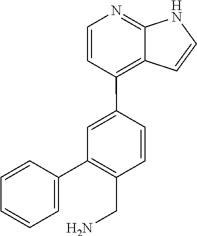 [SMILES: NCc1ccc(-c2ccnc3[nH]ccc23)cc1-c1ccccc1] | 378 | 15 | 20 | | |
| 247 | 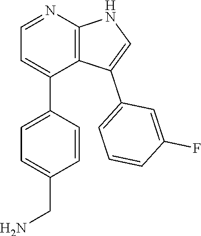 [SMILES: NCc1ccc(-c2ccnc3[nH]cc(-c4cccc(F)c4)c23)cc1] | 1410 | 21 | 11 | | |
| 248 | 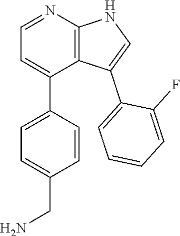 [SMILES: NCc1ccc(-c2ccnc3[nH]cc(-c4ccccc4F)c23)cc1] | 2840 | 50 | 10 | | |
| 249 | 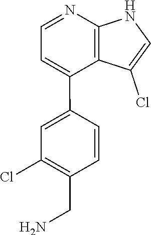 [SMILES: NCc1ccc(-c2ccnc3[nH]cc(Cl)c23)cc1Cl] | 218 | 8.4 | 7.9 | | |
| 250 | 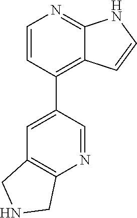 [SMILES: c1cc(-c2cnc3c(c2)CNC3)c2cc[nH]c2n1] | | | | | |
| 251 | 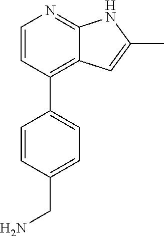 [SMILES: Cc1cc2c(-c3ccc(CN)cc3)ccnc2[nH]1] | >10000 | 893 | 389 | | |
| 252 | 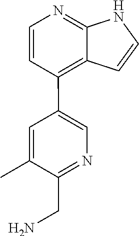 [SMILES: Cc1cc(-c2ccnc3[nH]ccc23)cnc1CN] | 3160 | 8.2 | 12 | | |
| 253 | 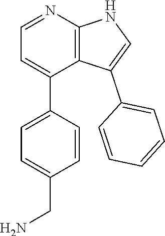 [SMILES: NCc1ccc(-c2ccnc3[nH]cc(-c4ccccc4)c23)cc1] | 983 | 13 | 5 | 338 | 2980 |
| 254 | 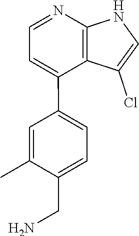 [SMILES: Cc1cc(-c2ccnc3[nH]cc(Cl)c23)ccc1CN] | 116 | 3.4 | 5.2 | 3910 | 714 |
| 255 | 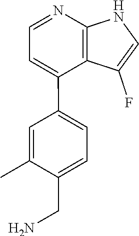 [SMILES: Cc1cc(-c2ccnc3[nH]cc(F)c23)ccc1CN] | 312 | 3.7 | 5.5 | 22600 | 6370 |
| 256 | 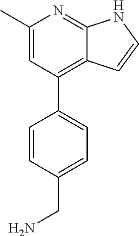 [SMILES: Cc1cc(-c2ccc(CN)cc2)c2cc[nH]c2n1] | >10000 | 9000 | 4260 | | |
| 257 | 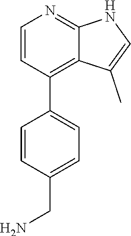 [SMILES: Cc1c[nH]c2nccc(-c3ccc(CN)cc3)c12] | 625 | 13 | 12 | | |
| 258 | 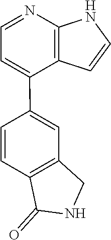 [SMILES: O=C1NCc2cc(-c3ccnc4[nH]ccc34)ccc21] | >10000 | 231 | 65 | | |
| 259 | 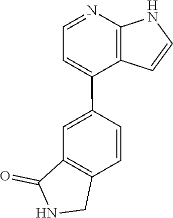 [SMILES: O=C1NCc2ccc(-c3ccnc4[nH]ccc34)cc21] | >10000 | 110 | 253 | | |
| 260 | 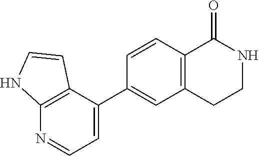 [SMILES: O=C1NCCc2cc(-c3ccnc4[nH]ccc34)ccc21] | 6600 | 87 | 52 | | |
| 261 | 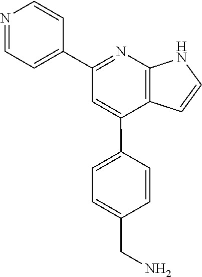 [SMILES: NCc1ccc(-c2cc(-c3ccncc3)nc3[nH]ccc23)cc1] | 8830 | 34 | 10 | | |
| 262 | 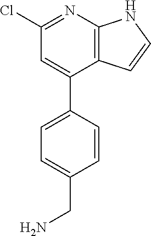 [SMILES: NCc1ccc(-c2cc(Cl)nc3[nH]ccc23)cc1] | >10000 | >10000 | 4420 | | |
| 263 | 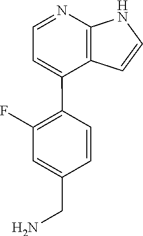 [SMILES: NCc1ccc(-c2ccnc3[nH]ccc23)c(F)c1] | 1190 | 34 | 72 | | |
| 264 | 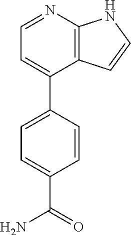 [SMILES: NC(=O)c1ccc(-c2ccnc3[nH]ccc23)cc1] | >10000 | 343 | 207 | | |
| 265 | 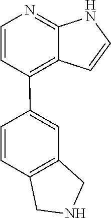 [SMILES: c1cc(-c2ccc3c(c2)CNC3)c2cc[nH]c2n1] | 517 | 14 | 13 | | |
| 266 | 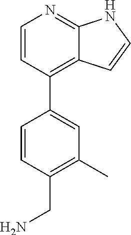 [SMILES: Cc1cc(-c2ccnc3[nH]ccc23)ccc1CN] | 249 | 6 | 5 | >50000 | 6710 |
| 267 | 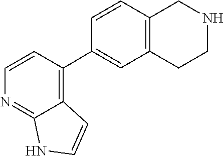 [SMILES: c1cc(-c2ccc3c(c2)CCNC3)c2cc[nH]c2n1] | 308 | 11 | 10 | 17300 | >50000 |
| 268 | 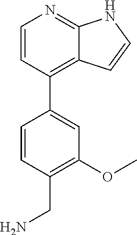 [SMILES: COc1cc(-c2ccnc3[nH]ccc23)ccc1CN] | 618 | 24 | 24 | | |
| 269 | 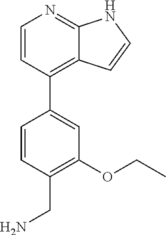 [SMILES: CCOc1cc(-c2ccnc3[nH]ccc23)ccc1CN] | 789 | 40 | 36 | | |
| 270 | 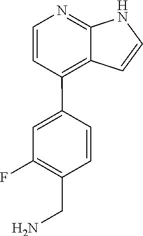 [SMILES: NCc1ccc(-c2ccnc3[nH]ccc23)cc1F] | 874 | 28 | 34 | | |
| 271 | 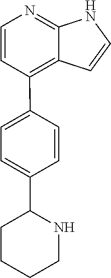 [SMILES: c1cc(-c2ccc(C3CCCCN3)cc2)c2cc[nH]c2n1] | 428 | 20 | 25 | 14500 | >50000 |
| 272 | 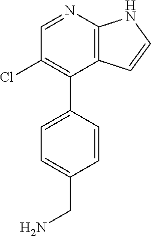 [SMILES: NCc1ccc(-c2c(Cl)cnc3[nH]ccc23)cc1] | 3100 | 447 | 268 | | |
| 273 | 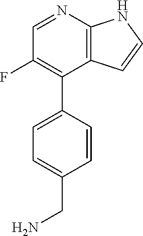 [SMILES: NCc1ccc(-c2c(F)cnc3[nH]ccc23)cc1] | 1390 | 90 | 148 | | |
| 274 | 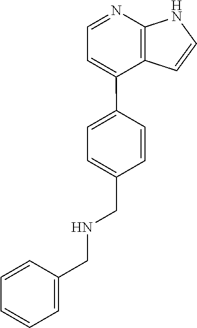 [SMILES: c1ccc(CNCc2ccc(-c3ccnc4[nH]ccc34)cc2)cc1] | 2432 | 36.6 | 60.5 | | |
| 275 | 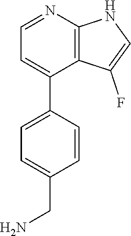 [SMILES: NCc1ccc(-c2ccnc3[nH]cc(F)c23)cc1] | 266 | 13 | 8.4 | 2060 | 10100 |
| 276 | 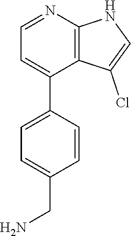 [SMILES: NCc1ccc(-c2ccnc3[nH]cc(Cl)c23)cc1] | 167 | 12 | 5.8 | 4140 | 2830 |
| 277 | 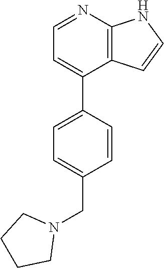 [SMILES: c1cc(-c2ccc(CN3CCCC3)cc2)c2cc[nH]c2n1] | 2200 | 63 | 100 | | |
| 278 | 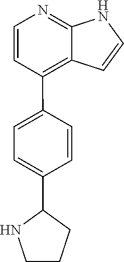 [SMILES: c1cc(-c2ccc(C3CCCN3)cc2)c2cc[nH]c2n1] | 321 | 17 | 33 | | |
| 279 | 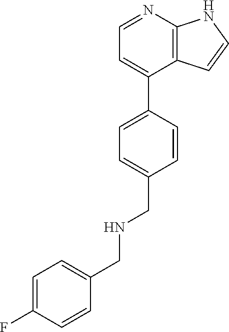 [SMILES: Fc1ccc(CNCc2ccc(-c3ccnc4[nH]ccc34)cc2)cc1] | 1081 | 9.8 | 29.3 | | |
| 280 | 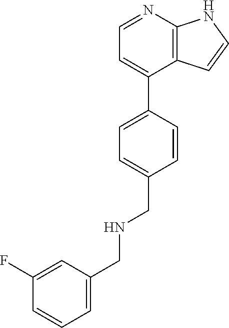 [SMILES: Fc1cccc(CNCc2ccc(-c3ccnc4[nH]ccc34)cc2)c1] | 858.8 | 10.8 | 25.6 | | |
| 281 | 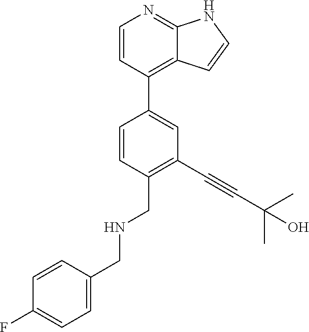 [SMILES: CC(C)(O)C#Cc1cc(-c2ccnc3[nH]ccc23)ccc1CNCc1ccc(F)cc1] | 4624 | 82.4 | 169.7 | | |
| 282 | 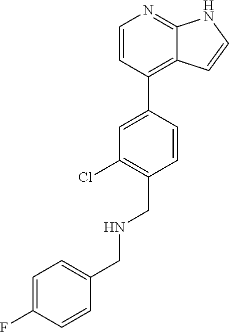 [SMILES: Fc1ccc(CNCc2ccc(-c3ccnc4[nH]ccc34)cc2Cl)cc1] | 3805 | 31.4 | 59.2 | | |
| 283 | 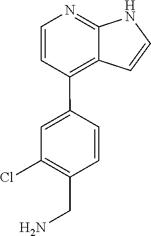 [SMILES: NCc1ccc(-c2ccnc3[nH]ccc23)cc1Cl] | 732 | 8 | 20 | | |
| 284 | 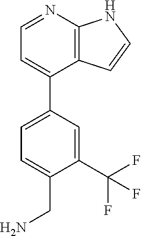 [SMILES: NCc1ccc(-c2ccnc3[nH]ccc23)cc1C(F)(F)F] | 1150 | 10 | 45 | >50000 | 34800 |
| 285 | 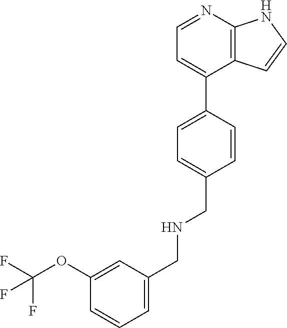 [SMILES: FC(F)(F)Oc1cccc(CNCc2ccc(-c3ccnc4[nH]ccc34)cc2)c1] | 1991 | 167 | 191 | | |
| 286 | 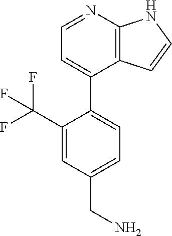 [SMILES: NCc1ccc(-c2ccnc3[nH]ccc23)c(C(F)(F)F)c1] | 3408 | 5053.4 | 1260 | | |
| 287 | 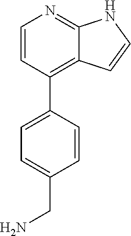 [SMILES: NCc1ccc(-c2ccnc3[nH]ccc23)cc1] | 1030 | 23 | 34 | >50000 | 22800 |
| 300 | 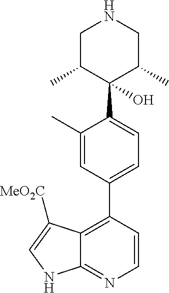 [SMILES: COC(=O)c1c[nH]c2nccc(-c3ccc([C@@]4(O)[C@H](C)CNC[C@@H]4C)c(C)c3)c12] | 282 | 3.2 | 1.6 | | |
| 301 | 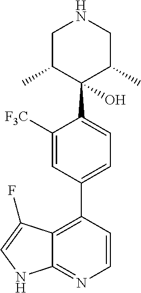 [SMILES: C[C@@H]1CNC[C@H](C)[C@@]1(O)c1ccc(-c2ccnc3[nH]cc(F)c23)cc1C(F)(F)F] | 143 | 11.1 | 10.7 | | |
| 302 | 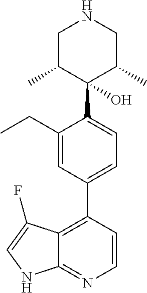 [SMILES: CCc1cc(-c2ccnc3[nH]cc(F)c23)ccc1[C@@]1(O)[C@H](C)CNC[C@@H]1C] | 39.5 | 8.7 | 4.9 | | |
| 303 | 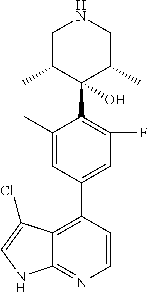 [SMILES: Cc1cc(-c2ccnc3[nH]cc(Cl)c23)cc(F)c1[C@@]1(O)[C@H](C)CNC[C@@H]1C] | 246 | 4.8 | 3.3 | | |
| 304 | 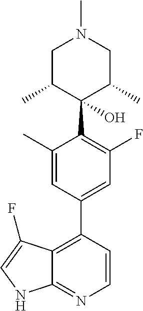 [SMILES: Cc1cc(-c2ccnc3[nH]cc(F)c23)cc(F)c1[C@@]1(O)[C@H](C)CN(C)C[C@@H]1C] | 271 | 5.1 | 4.0 | 26000 | 41.7 |
| 305 | 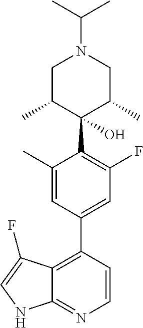 [SMILES: Cc1cc(-c2ccnc3[nH]cc(F)c23)cc(F)c1[C@@]1(O)[C@H](C)CN(C(C)C)C[C@@H]1C] | 758 | 4.1 | 3.7 | 29400 | 62.2 |
| 306 | 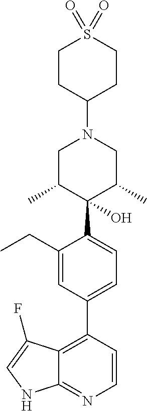 [SMILES: CCc1cc(-c2ccnc3[nH]cc(F)c23)ccc1[C@@]1(O)[C@H](C)CN(C2CCS(=O)(=O)CC2)C[C@@H]1C] | 31.2 | 11.6 | 5.4 | | |
| 307 | 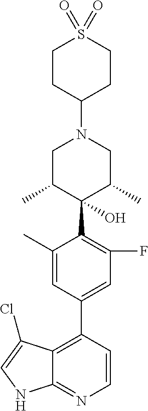 [SMILES: Cc1cc(-c2ccnc3[nH]cc(Cl)c23)cc(F)c1[C@@]1(O)[C@H](C)CN(C2CCS(=O)(=O)CC2)C[C@@H]1C] | 206 | 4.2 | 3.1 | | |
| 308 | 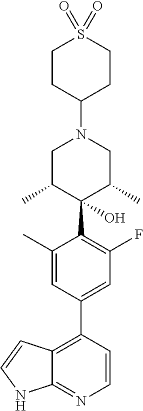 [SMILES: Cc1cc(-c2ccnc3[nH]ccc23)cc(F)c1[C@@]1(O)[C@H](C)CN(C2CCS(=O)(=O)CC2)C[C@@H]1C] | 1090 | 10.0 | 8.4 | | |
| 309 | 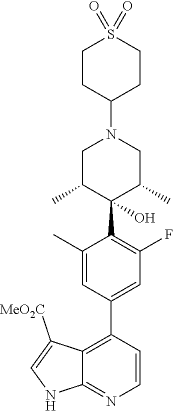 [SMILES: COC(=O)c1c[nH]c2nccc(-c3cc(C)c([C@@]4(O)[C@H](C)CN(C5CCS(=O)(=O)CC5)C[C@@H]4C)c(F)c3)c12] | 2270 | 3.1 | 3.0 | 39900 | 1350 |
| 310 | 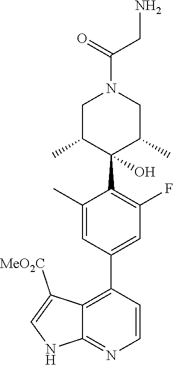 [SMILES: COC(=O)c1c[nH]c2nccc(-c3cc(C)c([C@@]4(O)[C@H](C)CN(C(=O)CN)C[C@@H]4C)c(F)c3)c12] | 4420 | 5.1 | 2.3 | 49400 | 4590 |
| 311 | 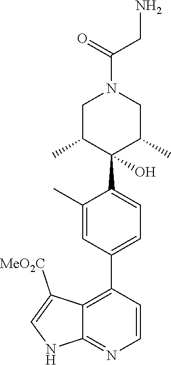 [SMILES: COC(=O)c1c[nH]c2nccc(-c3ccc([C@@]4(O)[C@H](C)CN(C(=O)CN)C[C@@H]4C)c(C)c3)c12] | 1040 | 6.9 | 3.6 | | |
| 312 | 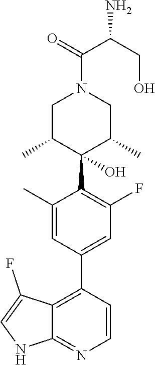 [SMILES: Cc1cc(-c2ccnc3[nH]cc(F)c23)cc(F)c1[C@@]1(O)[C@H](C)CN(C(=O)[C@H](N)CO)C[C@@H]1C] | 153 | 8.1 | 3.5 | 12800 | 10.4 |
| 313 | 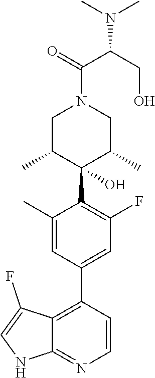 [SMILES: Cc1cc(-c2ccnc3[nH]cc(F)c23)cc(F)c1[C@@]1(O)[C@H](C)CN(C(=O)[C@@H](CO)N(C)C)C[C@@H]1C] | 312 | 4.4 | 1.6 | 19200 | 43 |
| 314 | 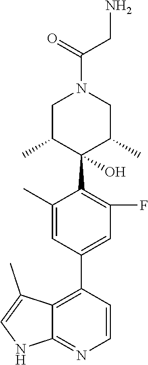 [SMILES: Cc1cc(-c2ccnc3[nH]cc(C)c23)cc(F)c1[C@@]1(O)[C@H](C)CN(C(=O)CN)C[C@@H]1C] | 249 | 1.6 | 4.8 | | |
| 315 | 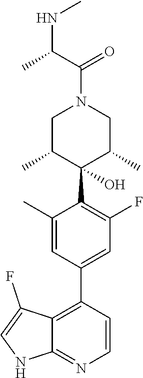 [SMILES: CN[C@@H](C)C(=O)N1C[C@@H](C)[C@](O)(c2c(C)cc(-c3ccnc4[nH]cc(F)c34)cc2F)[C@@H](C)C1] | 1350 | 11.6 | 5.7 | | |
| 316 | 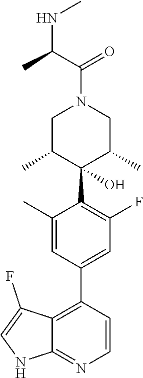 [SMILES: CN[C@H](C)C(=O)N1C[C@@H](C)[C@](O)(c2c(C)cc(-c3ccnc4[nH]cc(F)c34)cc2F)[C@@H](C)C1] | 1370 | 12.0 | 6.8 | | |
| 317 | 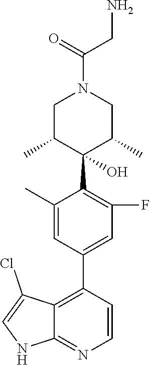 [SMILES: Cc1cc(-c2ccnc3[nH]cc(Cl)c23)cc(F)c1[C@@]1(O)[C@H](C)CN(C(=O)CN)C[C@@H]1C] | 186 | 7.6 | 3.8 | | |
| 318 | 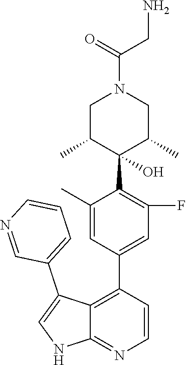 [SMILES: Cc1cc(-c2ccnc3[nH]cc(-c4cccnc4)c23)cc(F)c1[C@@]1(O)[C@H](C)CN(C(=O)CN)C[C@@H]1C] | 1420 | 4.1 | 1.3 | | |
| 319 | 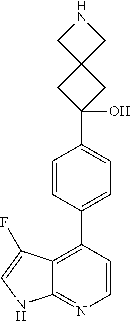 [SMILES: OC1(c2ccc(-c3ccnc4[nH]cc(F)c34)cc2)CC2(CNC2)C1] | 378 | 66.0 | 29.3 | 44800 | 27700 |

### Example 5: Dual LATS/AKT Inhibitors: General Methods

All commercially available reagents and solvents were purchased and used without further purification. All microwave reactions were carried out in a sealed microwave vial equipped with a magnetic stir bar and heated in a Biotage Initiator Microwave Synthesizer. ¹H NMR spectra were recorded on Varian 400 MHz spectrometers in CD₃OD, CD₃CN, CDCl₃, or D₆-DMSO as indicated. For spectra recorded in CD₃OD, chemical shifts are reported in ppm with CD₃OD (3.31 ppm) as reference for ¹H NMR spectra. For spectra recorded in CDCl₃, chemical shifts are reported in ppm relative to dueterochloroform (7.26 ppm for ¹H NMR). For spectra recorded in CD₃CN, chemical shifts are reported in ppm relative to CD₃CN (1.93 ppm for ¹H NMR). For spectra in D₆-DMSO chemical shifts are reported in ppm relative to D₆-DMSO (2.50 ppm for ¹H NMR). The coupling constants (J value) are reported as Hertz (Hz). The splitting patterns of the peaks were described as: singlet (s); doublet (d); triplet (t); quartet (q); multiplet (m) and septet (septet).

Compounds were analyzed on an Agilent 1200 series LC/MS equipped with a Luna C18 (3 mm×75 mm, 3 μm) reversed-phase column with UV detection at λ=220 nm and λ=254 nm. The mobile phase consisted of water containing 0.05% trifluoroacetic acid as component A and acetonitrile containing 0.025% trifluoroacetic acid as component B. A linear gradient was run as follows: 0 min 4% B; 7 min 100% B; 8 min 100% B at a flow rate of 0.8 ml/min.

Reverse phase chromatography for purification purposes was performed on a Waters semipreparative HPLC equipment. The column used was a Phenomenex Luna C18 (5 μm, 30×75 mm) at a flow rate of 45 mL/min. The mobile phase consisted of acetonitrile and water (each containing 0.1% trifluoroacetic acid, 0.1% formic acid, or 0.1% ammonium hydroxide, as indicated). A gradient of 5%-100% acetonitrile in water was used during the purification. Fraction collection was triggered by UV detection (220 nm).

### Example 6: Dual LATS/AKT Inhibitors: General Synthetic Protocols

Certain compounds of the invention were synthesized by the following general synthetic protocol

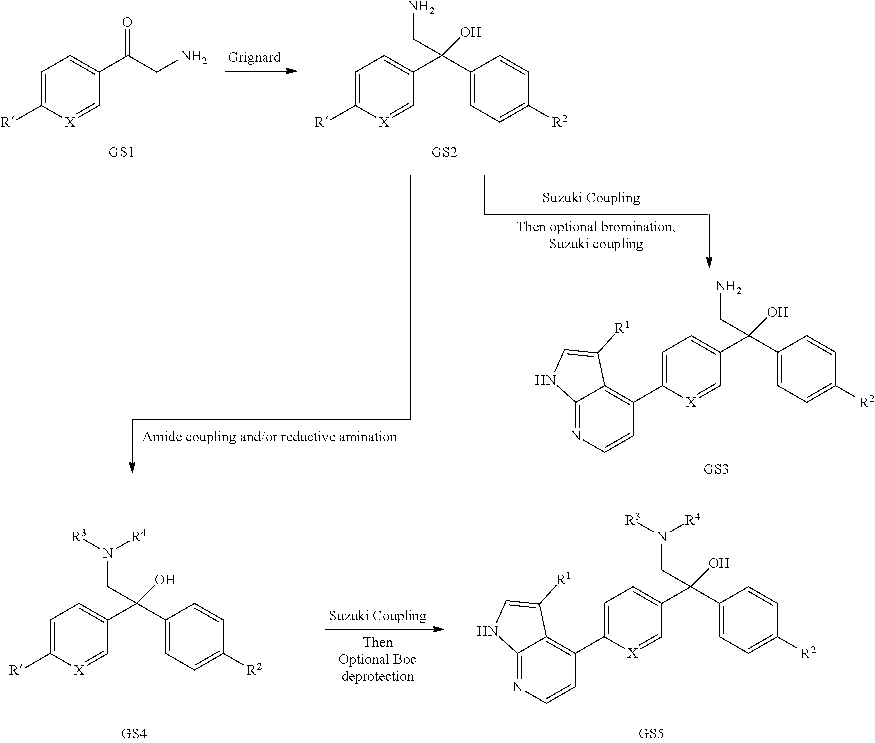

### Example 7: Dual LATS/AKT Inhibitors: Synthesis

### Synthesis of Compounds of General Formula Z1, Z2, and Z3

Compounds of general formula Z1, Z2, and Z3 can be prepared as illustrated in Scheme 6 below.

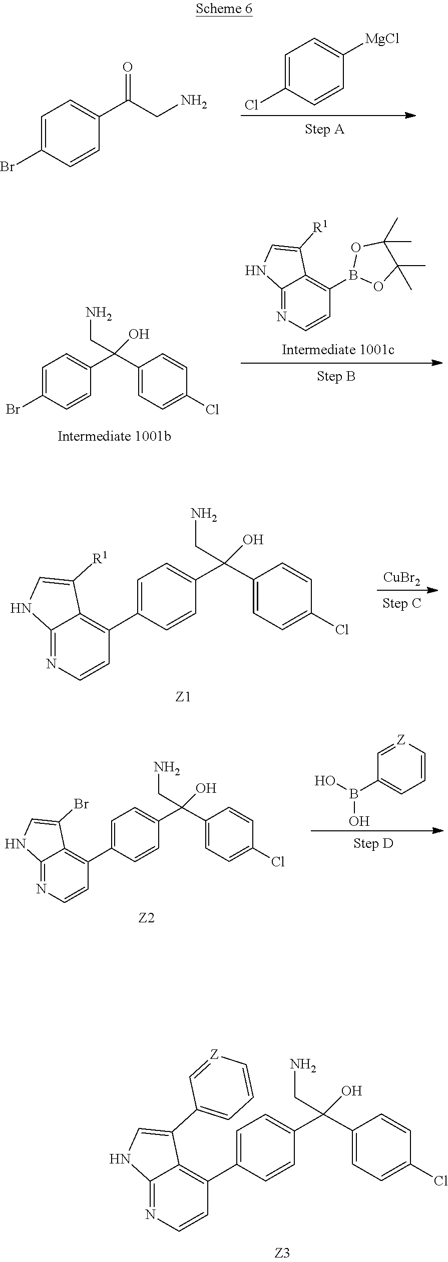

### Synthesis of Intermediates and Compounds of Scheme 6

### Intermediate 101b (Scheme 6): 2-amino-1-(4-bromophenyl)-1-(4-chlorophenyl)ethan-1-ol

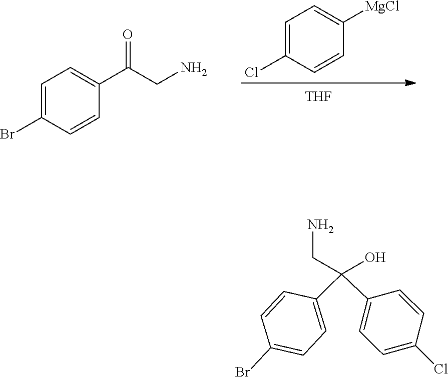

Step A: To a 20 mL microwave vial was added 2-amino-1-(4-bromophenyl)ethan-1-one (0.250 grams, 1.16 mmol) and a stirbar. The vial was sealed, and tetrahydrofuran (5 ml) was added via syringe. The vial was cooled to 0° C. with a water-ice bath, and (4-chlorophenyl)magnesium bromide in diethyl ether (5.0 mL, 1.0 M, 5.0 mmol) was added via syringe. The reaction was then heated in a microwave to 50° C. for 2 hours and stirred. The reaction was cooled, and poured into a solution of saturated aqueous sodium bicarbonate. The reaction mixture was then extracted with ethyl acetate, the organic phase was separated, and the solvent was removed by rotary evaporation. The crude product was purified by silica gel chromatography (0% to 15% methanol in dichloromethane) to give the title product. ¹H NMR (400 MHz, Methanol-d₄) δ 7.49-7.45 (m, 2H), 7.45-7.39 (m, 2H), 7.39-7.27 (m, 4H), 3.35 (s, 2H). LCMS (m/z [M+H]⁺): 328.0

### Intermediate 1001c (Scheme 6): 3-chloro-4-(4,4,5,5-tetramethyl-1,3,2-dioxaborolan-2-yl)-1H-pyrrolo[2,3-b]pyridine

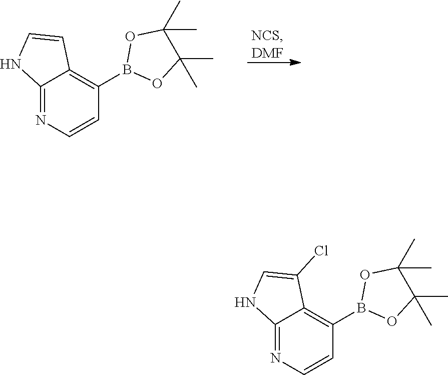

To a solution of 4-(4,4,5,5-tetramethyl-1,3,2-dioxaborolan-2-yl)-1H-pyrrolo[2,3-b]pyridine (0.600 grams, 2.56 mmol) in dimethyl formamide (5.0 ml) was added 1-chloropyrrolidine-2,5-dione (0.328 g, 2.46 mmol). The reaction was stirred for room temperature for 16 hours. It was then poured into diethyl ether and washed with water. The organic phase was separated, and the solvent removed by rotary evaporation. Purification by silica gel chromatography (0% to 100% ethyl acetate in hexanes) gave the title product. ¹H NMR (400 MHz, Chloroform-d) δ 11.07 (s, 1H), 8.31 (d, J=36.3 Hz, 1H), 7.50-7.31 (m, 2H), 1.44 (d, J=1.2 Hz, 12H).

### Compound 1001

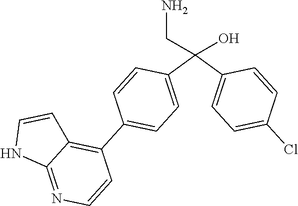 [SMILES: NCC(O)(c1ccc(Cl)cc1)c1ccc(-c2ccnc3[nH]ccc23)cc1]

### 1-(4-(1H-pyrrolo[2,3-b]pyridin-4-yl)phenyl)-2-amino-1-(4-chlorophenyl)ethan-1-ol

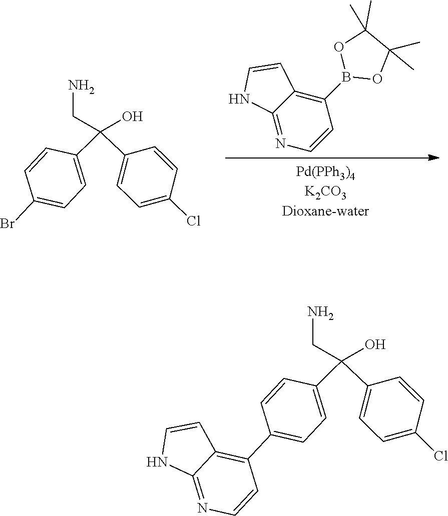 [SMILES: NCC(O)(c1ccc(Cl)cc1)c1ccc(Br)cc1 ; CC1(C)OB(c2ccnc3[nH]ccc23)OC1(C)C ; NCC(O)(c1ccc(Cl)cc1)c1ccc(-c2ccnc3[nH]ccc23)cc1]

Step B: To a 2 mL microwave vial containing a stirbar was added 2-amino-1-(4-bromophenyl)-1-(4-chlorophenyl)ethan-1-ol (intermediate 1001b) (0.060 grams, 0.184 mmol), followed by 4-(4,4,5,5-tetramethyl-1,3,2-dioxaborolan-2-yl)-1H-pyrrolo[2,3-b]pyridine (0.060 grams, 0.264 mmol), potassium carbonate (0.100 grams, 0.724 mmol) and tetrakis(triphenylphosphine)-palladium(0) (0.015 grams, 0.013 mmol). The vial was sealed, and 1,4-dioxane (1.5 ml) followed by water (0.5 ml) was added. The reaction mixture had nitrogen gas bubbled through it for 5 minutes at room temperature. It was then heated by microwave irradiation to 150° C. for 30 minutes. The reaction was cooled and poured into aqueous saturated sodium bicarbonate. It was extracted into ethyl acetate and the organic phase was separated and the solvent removed by rotary evaporation to give the crude product. Purification by reverse phase chromatography (5% to 100% acetonitrile in water with 0.1% trifluoracetic acid) gave the title product ¹H NMR (400 MHz, DMSO-d₆) δ 11.85 (s, 1H), 8.28 (d, J=5.0 Hz, 1H), 7.78 (d, J=8.0 Hz, 5H), 7.66 (d, J=8.2 Hz, 2H), 7.64-7.54 (i, 3H), 7.46 (d, J=8.5 Hz, 2H), 7.19 (d, J=5.0 Hz, 1H), 6.66 (s, 1H), 6.64-6.57 (m, 1H), 3.85-3.46 (i, 2H). LCMS (m/z [M+H]⁺): 364.1

### Compounds 1002-1003

Compounds 1002-1003 were prepared using an analogous procedure to that of Compound 1001 and are shown in Table 29.

TABLE 29

| Compound | Structure | IUPAC name | ¹H NMR | LCMS m/z |
|---|---|---|---|---|
| 1002 | 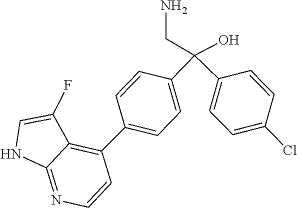 [SMILES: NCC(O)(c1ccc(Cl)cc1)c1ccc(-c2ccnc3[nH]cc(F)c23)cc1] | 2-amino-1-(4- chlorophenyl)-1- (4-(3-fluoro-1H- pyrrolo[2,3- b]pyridin-4- yl)phenyl)ethan- 1-ol | ¹H NMR (400 MHz, DMSO-d₆) δ 11.68 (d, J = 2.6 Hz, 1 H), 8.31 (d, J = 4.9 Hz, 1 H), 7.78 (d, J = 6.7 Hz, 3 H), 7.70 − 7.61 (m, 4 H), 7.58 (d, J = 8.6 Hz, 2 H), 7.53 (t, J = 2.4 Hz, 1 H), 7.50 − 7.43 (m, 2 H), 7.13 (d, J = 4.9 Hz, 1 H), 6.66 (s, 1 H), 3.90 − 3.79 (m, 1 H), 3.70 (d, J = 8.2 Hz, 1 H) | LCMS (m/z [M + H]⁺): 382.1 |
| 1003 | 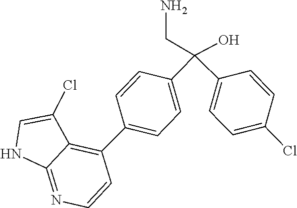 [SMILES: NCC(O)(c1ccc(Cl)cc1)c1ccc(-c2ccnc3[nH]cc(Cl)c23)cc1] | 2-amino-1-(4-(3- chloro-1H- pyrrolo[2,3- b]pyridin-4- yl)phenyl)-1-(4- chlorophenyl) ethan-1-ol | ¹H NMR (400 MHz, Methanol-d₄) δ 8.32 (d, J = 5.2 Hz, 1 H), 7.61 (s, 4 H), 7.59 − 7.52 (m, 3 H), 7.52 − 7.45 (m, 2 H), 7.45 − 7.34 (m, 4 H), 7.14 (d, J = 5.2 Hz, 1 H), 3.85 (q, J = 12.7 Hz, 2 H). | LCMS (m/z [M + H]⁺): 398.1 |

### Compound 1004

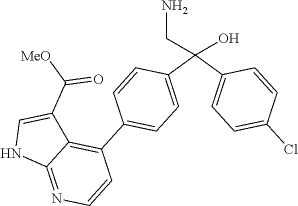 [SMILES: COC(=O)c1c[nH]c2nccc(-c3ccc(C(O)(CN)c4ccc(Cl)cc4)cc3)c12]

### Methyl 4-(4-(2-amino-1-(4-chlorophenyl)-1-hydroxyethyl)phenyl)-1H-pyrrolo[2,3-b]pyridine-3-carboxylate

Step B: To a 2 mL microwave vial containing a stirbar was added 2-amino-1-(4-bromophenyl)-1-(4-chlorophenyl)ethan-1-ol (0.050 grams, 0.184 mmol), followed by methyl 4-(4,4,5,5-tetramethyl-1,3,2-dioxaborolan-2-yl)-1H-pyrrolo[2,3-b]pyridine-3-carboxylate (0.043 grams, 0.264 mmol), potassium phosphate (0.100 grams, 0.724 mmol) and chloro(2-dicyclohexylphosphino-2′,4′,6′-triisopropyl-1,1′-biphenyl)[2-(2′-amino-1,1′-biphenyl)]palladium(II) (Xphos-Pd-G2) (0.015 grams, 0.019 mmol). The vial was sealed, and tetrahydrofuran (1.0 ml) followed by water (1.0 ml) was added. The reaction mixture had nitrogen gas bubbled through it for 5 minutes at room temperature. It was then heated by microwave irradiation to 85° C. for 45 minutes, then 100° for a further 45 minutes. The reaction was cooled, and the solvent was removed by rotary evaporation to give the crude product. The reaction was then dissolved in methanol and filtered. Purification by reverse phase chromatography (5% to 100% acetonitrile in water with 0.1% trifluoracetic acid) gave the title product. LCMS (m/z [M+H]⁺): 422.0

### Compound 1005

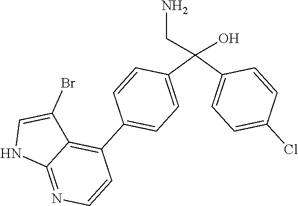 [SMILES: NCC(O)(c1ccc(Cl)cc1)c1ccc(-c2ccnc3[nH]cc(Br)c23)cc1]

### 2-amino-1-(4-(3-bromo-1H-pyrrolo[2,3-b]pyridin-4-yl)phenyl)-1-(4-chlorophenyl)ethan-1-ol

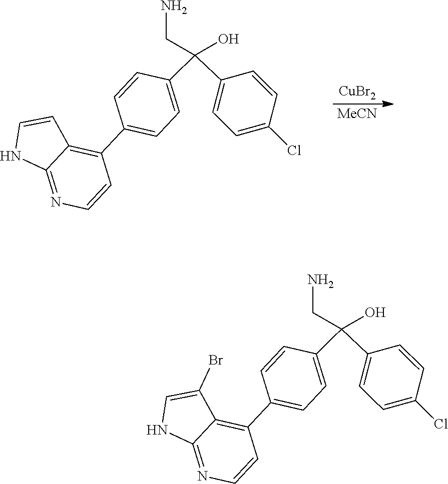

Step C: To a vial containing a stirbar was added 2-amino-1-(4-(3-bromo-1H-pyrrolo[2,3-b]pyridin-4-yl)phenyl)-1-(4-chlorophenyl)ethan-1-ol (107 mg, 0.294 mmol). Acetonitrile (10 ml) was added to the vial, followed by copper(II) bromide (197 mg, 882 mmol). The reaction was stirred for 10 minutes, then ammonia in methanol (7 N, 10 mL) was added and the reaction was stirred a further 30 minutes. The reaction was then poured into saturated aqueous sodium bicarbonate and extracted into ethyl acetate. The organic phase was separated and the solvent removed by rotary evaporation to give the crude product. Purification by reverse phase chromatography (5% to 100% acetonitrile in water with 0.1% trifluoroaceitic acid) gave the title product. ¹H NMR (400 MHz, DMSO-d₆) δ 12.27 (s, 1H), 8.30 (d, J=4.9 Hz, 1H), 7.78 (s, 3H), 7.73 (d, J=2.7 Hz, 1H), 7.58 (t, J=8.9 Hz, 4H), 7.52 (d, J=8.1 Hz, 2H), 7.46 (d, J=8.5 Hz, 2H), 6.99 (d, J=4.8 Hz, 1H), 6.66 (s, 1H), 3.96-3.77 (m, 1H), 3.67 (t, J=7.0 Hz, 1H). LCMS (m/z [M+H]⁺): 444.0

### Compound 1006

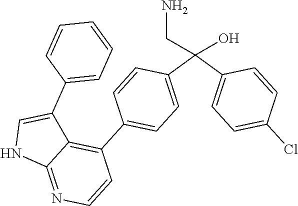 [SMILES: NCC(O)(c1ccc(Cl)cc1)c1ccc(-c2ccnc3[nH]cc(-c4ccccc4)c23)cc1]

### 2-amino-1-(4-chlorophenyl)-1-(4-(3-phenyl-1H-pyrrolo[2,3-b]pyridin-4-yl)phenyl)ethan-1-ol

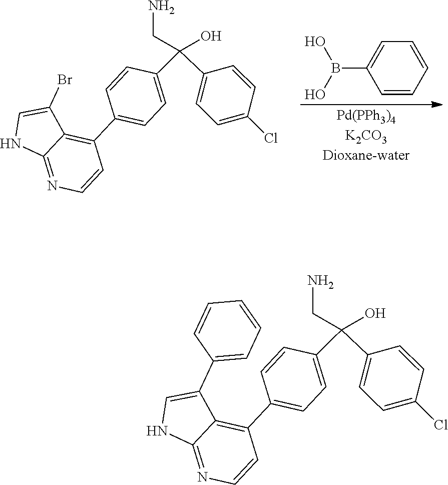

Step D: To a 2 mL microwave vial containing a stirbar was added 2-amino-1-(4-(3-bromo-1H-pyrrolo[2,3-b]pyridin-4-yl)phenyl)-1-(4-chlorophenyl)ethan-1-ol (0.050 grams, 0.113 mmol), followed by phenylboronic acid (0.030 grams, 0.264 mmol), potassium carbonate (0.100 grams, 0.724 mmol) and tetrakis(triphenylphosphine)-palladium(0) (0.010 grams, 0.077 mmol). The vial was sealed, and 1,4-dioxane (1.5 ml) followed by water (0.5 ml) was added. The reaction mixture had nitrogen gas bubbled through it for 5 minutes at room temperature. It was then heated by microwave irradiation to 150° C. for 30 minutes. The reaction was cooled and the solvent was removed by rotary evaporation. Purification by reverse phase chromatography (5% to 100% acetonitrile in water with 0.1% trifluoroacetic acid) gave the title product. ¹H NMR (400 MHz, Methanol-d₄) δ 8.34 (d, J=5.3 Hz, 1H), 7.82 (dd, J=47.7, 8.1 Hz, 1H), 7.74-7.56 (m, 2H), 7.53 (s, 1H), 7.47 (d, J=1.1 Hz, 4H), 7.31-7.17 (m, 4H), 6.94-6.78 (m, 4H), 3.72 (q, J=12.8 Hz, 2H). LCMS (m/z [M+H]⁺): 440.1

### Compound 1007

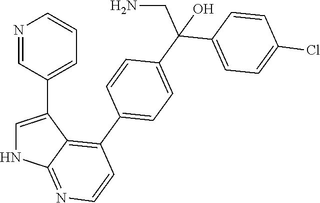 [SMILES: NCC(O)(c1ccc(Cl)cc1)c1ccc(-c2ccnc3[nH]cc(-c4cccnc4)c23)cc1]

### 2-amino-1-(4-chlorophenyl)-1-(4-(3-(pyridin-3-yl)-1H-pyrrolo[2,3-b]pyridin-4-yl)phenyl)ethan-1-ol

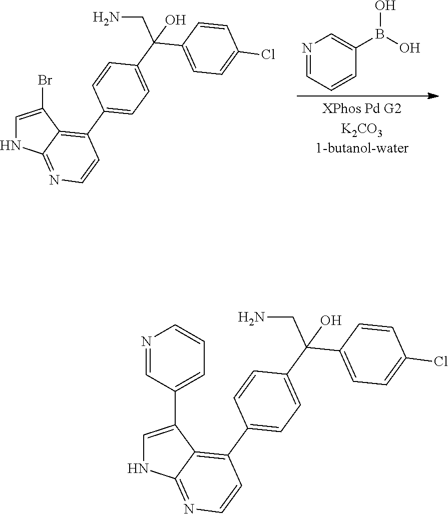 [SMILES: NCC(O)(c1ccc(Cl)cc1)c1ccc(-c2ccnc3[nH]cc(Br)c23)cc1 ; OB(O)c1cccnc1 ; NCC(O)(c1ccc(Cl)cc1)c1ccc(-c2ccnc3[nH]cc(-c4cccnc4)c23)cc1]

Step D: To a 2 mL microwave vial containing a stirbar was added 2-amino-1-(4-(3-bromo-1H-pyrrolo[2,3-b]pyridin-4-yl)phenyl)-1-(4-chlorophenyl)ethan-1-ol (0.043 grams, 0.097 mmol), followed by pyridin-3-ylboronic acid (0.015 grams, 0.122 mmol), potassium carbonate (0.100 grams, 0.724 mmol) and chloro(2-dicyclohexylphosphino-2′,4′,6′-triisopropyl-1,1′-biphenyl)[2-(2′-amino-1,1′-biphenyl)]palladium(II) (Xphos-Pd-G2) (0.010 grams, 0.013 mmol). The vial was sealed and 1-butanol (1.5 ml) followed by water (0.5 ml) was added. The reaction mixture had nitrogen gas bubbled through it for 5 minutes at room temperature. It was then heated by microwave irradiation to 80° C. for 30 minutes and then to 100° C. for 30 minutes. The reaction was cooled and the solvent was removed by rotary evaporation. Purification by reverse phase chromatography (5% to 100% acetonitrile in water with 0.1% trifluoroacetic acid) gave the title product. ¹H NMR (400 MHz, DMSO-d₆) δ 12.28 (s, 1H), 8.32 (dd, J=34.3, 5.0 Hz, 1H), 8.26-8.02 (m, 2H), 7.86-7.64 (m, 6H), 7.51 (p, J=9.0, 8.4 Hz, 4H), 7.27 (d, J=8.1 Hz, 2H), 7.20 (d, J=7.8 Hz, 1H), 7.19-7.02 (m, 4H), 6.87 (s, 1H), 6.55 (s, 1H), 3.66 (s, 2H). LCMS (m/z [M+H]⁺): 441.2

### Synthesis of Compounds of General Formula Y1 and Y2

Compounds of formula Y1 and Y2 can be prepared as illustrated in Scheme 7 below.

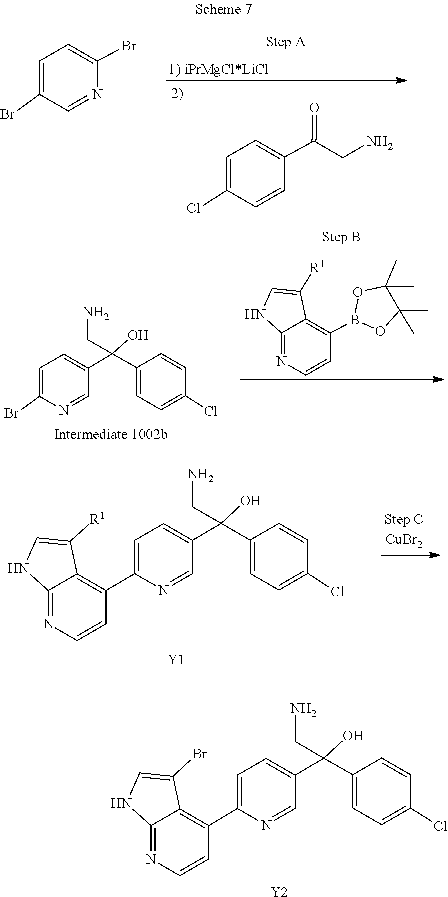

### Synthesis of Intermediates and Compounds of Scheme 7

### Intermediate 1002b (Scheme 7): 2-amino-1-(6-bromopyridin-3-yl)-1-(4-chlorophenyl)ethan-1-ol

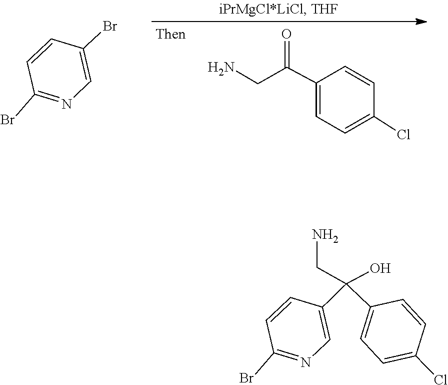 [SMILES: Brc1ccc(Br)nc1 ; NCC(=O)c1ccc(Cl)cc1 ; NCC(O)(c1ccc(Cl)cc1)c1ccc(Br)nc1]

Step A: To a solution of 2,5-dibromopyridine (1.2 g, 4.8 mmol) in THE (6.0 ml) under a nitrogen balloon, held at 0° C. with an ice bath was added isopropyl magnesium chloride lithium chloride solution in THE (1.0 M, 3.9 ml, 4.8 mmol). The solution was stirred at room temperature for 2 h. In a separate vial, 2-amino-1-(4-chlorophenyl)ethan-1-one, HCl (0.200 g, 0.97 mmol) was added, and dissolved in THE (4.0 ml). The vial was sealed. The above Grignard solution was then added to the solution containing the ketone via syringe at 0° C. The reaction was then heated to 50° C. for 3 hours, by microwave irradiation. The reaction was cooled and quenched with excess aqueous sodium bicarbonate. The solution was extracted with ethyl acetate, and the organic layer was dried over Na₂SO₄ and concentrated under reduced pressure. Purification by reverse phase chromatography (0% to 100% acetonitrile in water with 0.1% trifluoroacetic acid) gave the title product. LCMS (m/z [M+H]⁺): 328.0

### Compound 1008

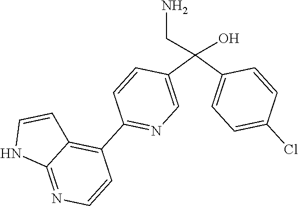 [SMILES: NCC(O)(c1ccc(Cl)cc1)c1ccc(-c2ccnc3[nH]ccc23)nc1]

### 1-(6-(1H-pyrrolo[2,3-b]pyridin-4-yl)pyridin-3-yl)-2-amino-1-(4-chlorophenyl)ethan-1-ol

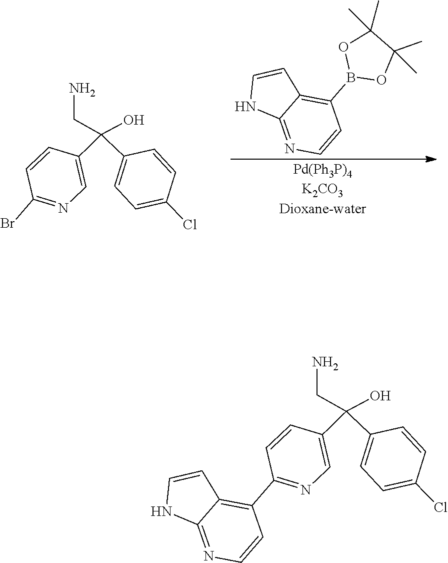

Step B: To a stirred solution of 2-amino-1-(6-bromopyridin-3-yl)-1-(4-chlorophenyl)ethan-1-ol (0.050 grams, 0.153 mmol) and 4-(4,4,5,5-tetramethyl-1,3,2-dioxaborolan-2-yl)-1H-pyrrolo[2,3-b]pyridine (0.0374 g, 0.153 mmol) in dioxane:water (3.5:0.5 ml) was added potassium carbonate (0.064 g, 0.46 mmol) and the reaction mixture was purged with nitrogen gas for 15 min. Then tetrakis(triphenylphosphine)-palladium(0) (0.0177 g, 0.053 mmol) was added into the reaction mixture. The reaction vial was sealed, and heated at 150° C. by microwave irradiation for 30 min. The reaction was cooled, and the reaction mixture was concentrated under reduced pressure. It was then diluted with water and extracted with ethyl acetate. The organic layer was dried over sodium sulfate and concentrated under reduced pressure. Purification by reverse phase chromatography (5% to 100% acetonitrile in water with 0.1% formic acid) gave the title product. ¹H NMR (400 MHz, Methanol-d₄) δ 8.96 (s, 1H), 8.42 (q, J=2.4 Hz, 1H), 8.14 (dd, J=6.0, 5.5 Hz, 2H), 7.80 (t, J=6.3 Hz, 1H), 7.66 (t, J=3.8 Hz, 1H), 7.58 (d, J=8.3 Hz, 2H), 7.46 (d, J=8.4 Hz, 2H), 7.15 (q, J=3.6 Hz, 1H), 3.92 (q, J=13.1 Hz, 2H). LCMS (m/z [M+H]⁺) 364.8

### Compound 1009

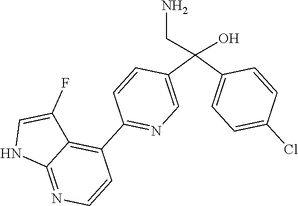 [SMILES: NCC(O)(c1ccc(Cl)cc1)c1ccc(-c2ccnc3[nH]cc(F)c23)nc1]

### 2-amino-1-(4-chlorophenyl)-1-(6-(3-fluoro-1H-pyrrolo[2,3-b]pyridin-4-yl)pyridin-3-yl)ethan-1-ol

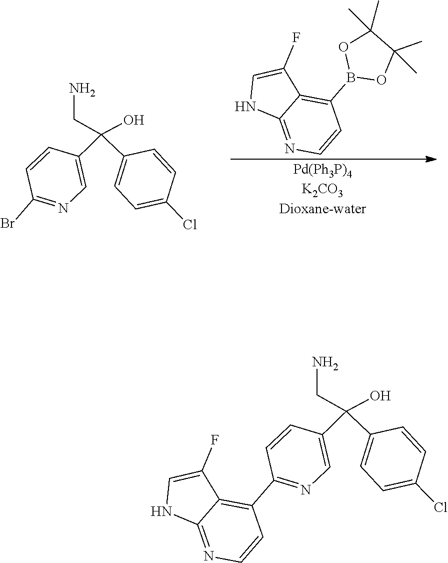

Stec B: To a stirred solution of 2-amino-1-(6-bromopyridin-3-yl)-1-(4-chlorophenyl)ethan-1-ol (0.115 g, 0.350 mmol) and 3-fluoro-4-(4,4,5,5-tetramethyl-1,3,2-dioxaborolan-2-yl)-1H-pyrrolo[2,3-b]pyridine (0.093 g, 0.350 mmol) in dioxane:water (3.5:0.5 ml) was added potassium carbonate (0.147 g, 1.05 mmol) and the reaction mixture was purged with nitrogen gas for 15 min. Then tetrakis(triphenylphosphine)-palladium(O) (0.041 g, 0.035 mmol) was added into the reaction mixture. The vial was sealed, and heated at 150° C. in microwave irradiation for 30 min. The reaction was cooled, and the reaction mixture was concentrated under reduced pressure. It was then diluted with water and extracted with ethyl acetate. The organic layer was dried over sodium sulfate and concentrated under reduced pressure. Purification by reverse phase chromatography (500 to 100% acetonitrile with 0.1% formic acid) gave the title product. 1H NMR (400 MHz, DMSO-d₆) δ 11.75 (s, 1H), 8.89 (s, 1H), 8.36 (d, J 4.8 Hz, 1H), 8.02 (d, J 6.4 Hz, 1H), 7.98-7.75 (m, 4H) 7.65-7.58 (m, 3H) 7.52-7.42 (m, 3H), 6.87 (s, 1H), 3.86-3.78 (in, 2H).). LCMS (m/z [M+H]⁺) 382.8

### Compound 1010

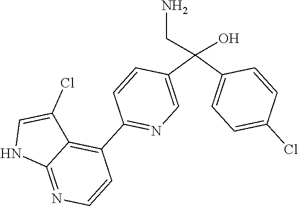 [SMILES: NCC(O)(c1ccc(Cl)cc1)c1ccc(-c2ccnc3[nH]cc(Cl)c23)nc1]

### 2-amino-1-(6-(3-chloro-1H-pyrrolo[2,3-b]pyridin-4-yl)pyridin-3-yl)-1-(4-chlorophenyl)ethan-1-ol

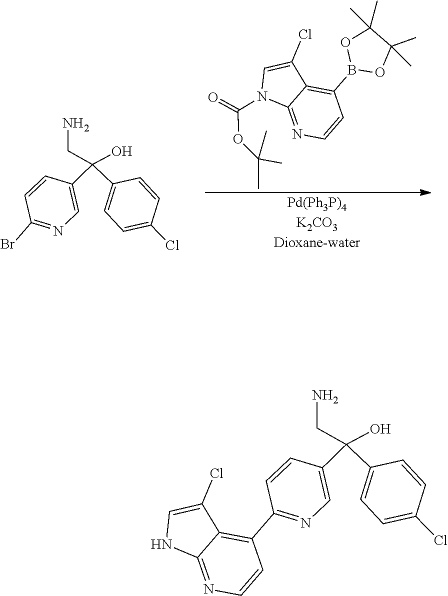 [SMILES: NCC(O)(c1ccc(Cl)cc1)c1ccc(Br)nc1 ; CC(C)(C)OC(=O)n1cc(Cl)c2c(B3OC(C)(C)C(C)(C)O3)ccnc21 ; "Pd(Ph3P)4K2CO3Dioxane-water" ; NCC(O)(c1ccc(Cl)cc1)c1ccc(-c2ccnc3[nH]cc(Cl)c23)nc1]

Step B: To a stirred solution of 2-amino-1-(6-bromopyridin-3-yl)-1-(4-chlorophenyl)ethan-1-ol (0.233 g, 0.714 mmol) and tert-butyl 3-chloro-4-(4,4,5,5-tetramethyl-1,3,2-dioxaborolan-2-yl)-1H-pyrrolo[2,3-b]pyridine-1-carboxylate (0.270 g, 0.714 mmol) in dioxane:water (4.5:0.5 ml) was added potassium carbonate (0.296 g, 0.00214 mol) and the reaction mixture was purged with nitrogen gas for 15 min. Then tetrakis(triphenylphosphine)-palladium(0) (0.083 g, 0.071 mmol) was added into the reaction mixture and heated at 150° C. in microwave for 30 min. The reaction mixture was concentrated under reduced pressure, diluted with water and extracted with ethyl acetate. The organic layer was dried with sodium sulfate and concentrated under reduced pressure. Purification by reverse phase chromatography (5% to 100% acetonitrile with 0.1% formic acid) gave the title product. ¹H NMR (400 MHz, DMSO-d₆) δ 8.80 (dd, J=9.2, 2.3 Hz, 1H), 8.35 (d, J=4.8 Hz, 1H), 8.25 (s, 1H), 8.12-7.86 (m, 2H), 7.72 (d, J=1.9 Hz, 1H), 7.58 (ddd, J=23.7, 8.4, 6.6 Hz, 3H), 7.42 (dt, J=8.7, 3.1 Hz, 2H), 7.19 (dd, J=4.9, 1.8 Hz, 1H), 4.03 (ddd, J=46.3, 13.7, 5.7 Hz, 1H), 3.45 (s, 1H). LCMS (m/z [M+H]⁺) 398.8

### Compound 1011

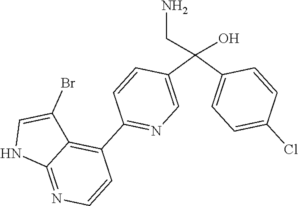 [SMILES: NCC(O)(c1ccc(Cl)cc1)c1ccc(-c2ccnc3[nH]cc(Br)c23)nc1]

### 2-amino-1-(6-(3-bromo-1H-pyrrolo[2,3-b]pyridin-4-yl)pyridin-3-yl)-1-(4-chlorophenyl)ethan-1-ol

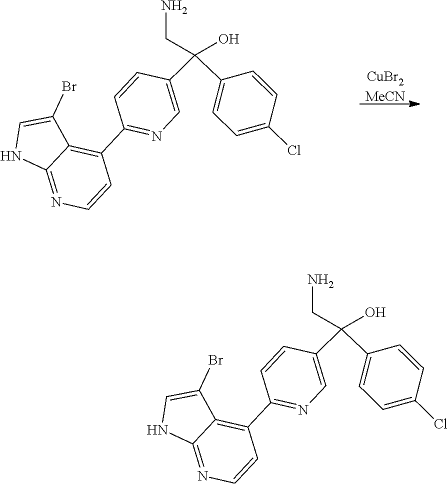 [SMILES: NCC(O)(c1ccc(Cl)cc1)c1ccc(-c2ccnc3[nH]cc(Br)c23)nc1 ; "CuBr2MeCN" ; NCC(O)(c1ccc(Cl)cc1)c1ccc(-c2ccnc3[nH]cc(Br)c23)nc1]

Step C: To a solution of 1-(6-(1H-pyrrolo[2,3-b]pyridin-4-yl)pyridin-3-yl)-2-amino-1-(4-chlorophenyl)ethan-1-ol (0.05 g, 0.14 mmol) in acetonitrile (3.0 ml) was added copper (II) bromide (0.092 g, 0.412 mmol). The reaction was stirred for 15 min. Then ammonia in methanol (5.0 ml, 7 N solution) was added. The resulting mixture was stirred for 30 min at room temperature. Then the reaction mixture was extracted with ethyl acetate and washed with aqueous sodium bicarbonate, then water. The organic layer dried over sodium sulfate and concentrated under reduced pressure. Purification by reverse phase chromatography (5% to 100% acetonitrile with 0.1% formic acid) gave the title product. ¹H NMR (400 MHz, DMSO-d₆) δ 8.80 (d, J=2.2 Hz, 1H), 8.34 (d, J=4.8 Hz, 1H), 8.23 (s, 1H), 7.92 (dd, J=8.0, 2.2 Hz, 1H), 7.74 (d, J=2.8 Hz, 1H), 7.57 (dd, J=12.9, 8.2 Hz, 3H), 7.42 (d, J=8.5 Hz, 2H), 7.15 (d, J=4.8 Hz, 1H), 3.44 (s, 2H). LCMS (m/z [M+H]⁺) 444.6

### Synthesis of Compound 1012 and Intermediates Thereof

Compound 1012 can be prepared as illustrated in Scheme 8 below.

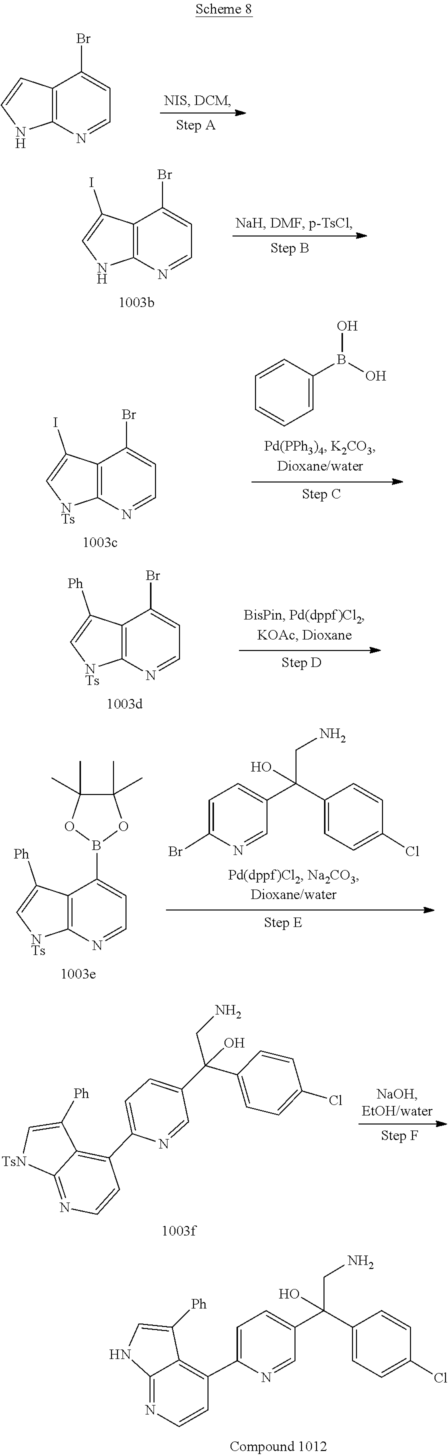

### Intermediate 1003b (Scheme 8): 4-bromo-3-iodo-1H-pyrrolo[2,3-b]pyridine

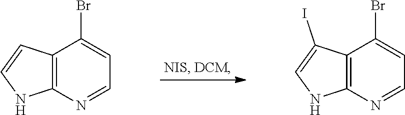 [SMILES: Brc1ccnc2[nH]ccc12 ; Brc1ccnc2[nH]cc(I)c12]

Step A: To a solution of 4-bromo-1H-pyrrolo[2,3-b]pyridine (1.5 g, 7.6 mmol) in dichloromethane (25 ml) under a nitrogen balloon, held at 0° C. with an ice bath was added N-iodosuccinimide (1.89 g, 8.3 mmol) and the reaction mixture was stirred at 0° C. for 1 h. The reaction mixture was concentrated and saturated aqueous sodium sulphite solution was added. The solid obtained was filtered off to yield the desired compound without further purification. LCMS (m/z [M+H]⁺) 322.8

### Intermediate 1003c (Scheme 8): 4-bromo-3-iodo-1-tosyl-1H-pyrrolo[2,3-b]pyridine

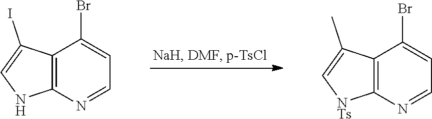 [SMILES: Brc1ccnc2[nH]cc(I)c12 ; Cc1ccc(S(=O)(=O)n2cc(C)c3c(Br)ccnc32)cc1]

Step B: To a solution of 4-bromo-3-iodo-1H-pyrrolo[2,3-b]pyridine (2.0 g, 6.2 mmol) in dimethylformamide (20 ml) under a nitrogen balloon, held at 0° C. with an ice bath was added sodium hydride (60% in mineral oil, 0.3 g, 7.5 mmol) and the reaction mixture was stirred at 0° C. for 30 min. Then, para-toluenesulfonyl chloride (1.4 g, 7.5 mmol) was added and the reaction mixture was stirred at 0° C. for 1.5 h. The reaction mixture was quenched with ice cold aqueous solution of ammonium chloride and extracted with ethyl acetate. The organic layer was further washed with water, dried with sodium sulfate and concentrated under reduced pressure. The crude was purified using silica gel column chromatography (5% ethyl acetate in hexanes) to yield the desired compound. LCMS (m/z [M+H]⁺): 476.9

### Intermediate 1003d (Scheme 8): 4-bromo-3-phenyl-1-tosyl-1H-pyrrolo[2,3-b]pyridine

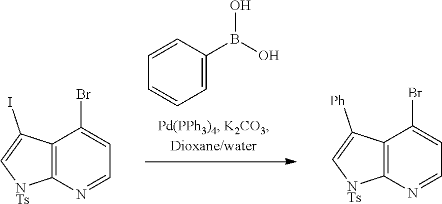 [SMILES: Cc1ccc(S(=O)(=O)n2cc(I)c3c(Br)ccnc32)cc1 ; OB(O)c1ccccc1 ; Cc1ccc(S(=O)(=O)n2cc(-c3ccccc3)c3c(Br)ccnc32)cc1]

Step C: To a stirred solution of 4-bromo-3-iodo-1-tosyl-1H-pyrrolo[2,3-b]pyridine (2.0 g, 4.2 mmol) and phenylboronic acid (0.512 g, 4.2 mol) in dioxane:water (30:6 ml) was added potassium carbonate (1.65 g, 12.6 mmol) and the reaction mixture was purged with nitrogen gas for 15 min. Then tetrakis(triphenylphosphine)-palladium(0) (0.485 g, 0.42 nmol) was added into the reaction mixture and heated at 75° C. for 20 h. The reaction mixture was concentrated under reduced pressure, diluted with water, and extracted with ethyl acetate. The organic layer was dried with sodium sulfate and concentrated under reduced pressure. The crude material was purified by silica gel column chromatography (2% ethyl acetate in hexanes) the title compound. LCMS (m/z [M+H]⁺): 429.0

### Intermediate 1003e (Scheme 8): 3-phenyl-4-(4,4,5,5-tetramethyl-1,3,2-dioxaborolan-2-yl)-1-tosyl-1H-pyrrolo[2,3-b]pyridine

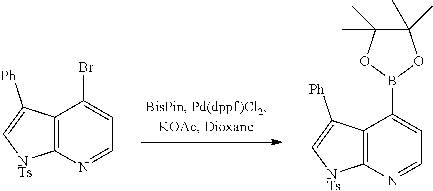 [SMILES: Cc1ccc(S(=O)(=O)n2cc(-c3ccccc3)c3c(Br)ccnc32)cc1 ; Cc1ccc(S(=O)(=O)n2cc(-c3ccccc3)c3c(B4OC(C)(C)C(C)(C)O4)ccnc32)cc1]

Step D: To a stirred solution of 4-bromo-3-phenyl-1-tosyl-1H-pyrrolo[2,3-b]pyridine (0.9 g, 2.1 mmol) and bis pinacolato diborane (1.07 g, 4.2 mmol) in dimethylformamide (18 ml) was added potassium acetate (0.617 g, 6.3 mmol) and the reaction mixture was purged with nitrogen gas for 15 min. Then [1,1′-bis(diphenylphosphino)ferrocene]dichloropalladium(II)-dichloromethane complex (0.171 g, 0.2.1 mmol) was added into the reaction mixture and heated at 90° C. for 4 h. After completion, the reaction mixture was diluted with ice cold water, treated with 2 N aqueous sodium hydroxide (10 ml) to pH 12 and extracted with ethyl acetate. The aqueous layer was acidified with 2 N aqueous hydrochloric acid (˜10 ml) to pH 1 and extracted with ethyl acetate. The organic layers were combined and dried with sodium sulfate and concentrated under reduced pressure. The crude material was purified by silica gel column chromatography (5% ethyl acetate in hexanes) to yield the title compound. LCMS (m/z [M+H]⁺): 475.1

### Intermediate 1003f (Scheme 8): 2-amino-1-(4-chlorophenyl)-1-(6-(3-phenyl-1-tosyl-1H-pyrrolo[2,3-b]pyridin-4-yl)pyridin-3-yl)ethan-1-ol

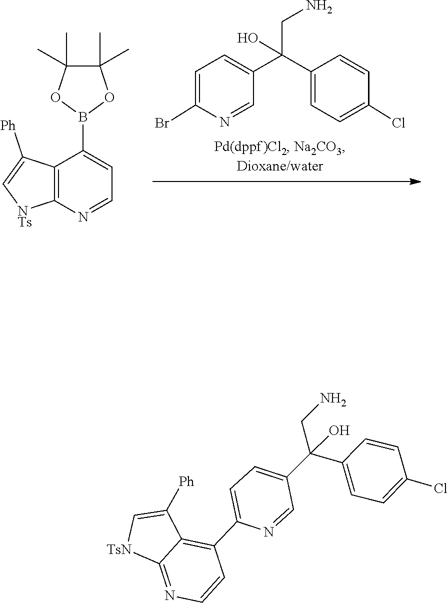 [SMILES: Cc1ccc(S(=O)(=O)n2cc(-c3ccccc3)c3c(B4OC(C)(C)C(C)(C)O4)ccnc32)cc1 ; NCC(O)(c1ccc(Cl)cc1)c1ccc(Br)nc1 ; Cc1ccc(S(=O)(=O)n2cc(-c3ccccc3)c3c(-c4ccc(C(O)(CN)c5ccc(Cl)cc5)cn4)ccnc32)cc1]

Step E: To a stirred solution of 2-amino-1-(6-bromopyridin-3-yl)-1-(4-chlorophenyl)ethan-1-ol (0.074 g, 0.426 mmol) and 3-phenyl-4-(4,4,5,5-tetramethyl-1,3,2-dioxaborolan-2-yl)-1-tosyl-1H-pyrrolo[2,3-b]pyridine (0.139 g, 0.295 mmol) in dioxane:water (3:1 ml) was added sodium carbonate (0.072 g, 0.00068 mol) and the reaction mixture was purged with nitrogen gas for 15 min. Then [1,1′-Bis(diphenylphosphino)ferrocene]dichloropalladium(II) (0.016 g, 0.0227 mmol) was added into the reaction mixture and heated at 90° C. in microwave for 30 min. The reaction mixture was diluted with water and extracted with ethyl acetate. The organic layer was dried with sodium sulfate and concentrated under reduced pressure. The crude material was purified by silica gel column chromatography (3% methanol in dichloromethane) to yield the title compound. LCMS (m/z [M+H]⁺): 595.2

### Compound 1012

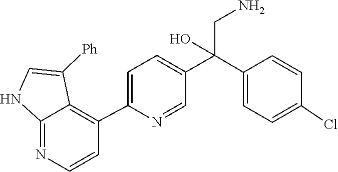 [SMILES: NCC(O)(c1ccc(Cl)cc1)c1ccc(-c2ccnc3[nH]cc(-c4ccccc4)c23)nc1]

### 2-amino-1-(4-chlorophenyl)-1-(6-(3-phenyl-1H-pyrrolo[2,3-b]pyridin-4-yl)pyridin-3-yl) ethan-1-ol

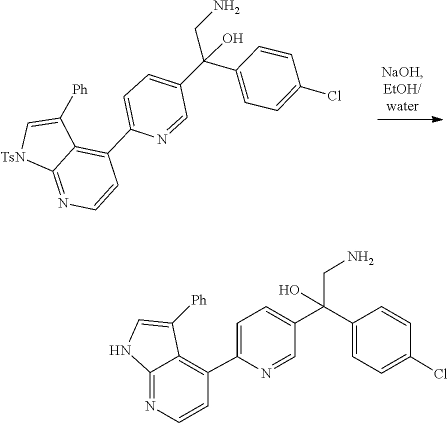 [SMILES: Cc1ccc(S(=O)(=O)n2cc(-c3ccccc3)c3c(-c4ccc(C(O)(CN)c5ccc(Cl)cc5)cn4)ccnc32)cc1 ; NCC(O)(c1ccc(Cl)cc1)c1ccc(-c2ccnc3[nH]cc(-c4ccccc4)c23)nc1]

Step F: To a solution of 2-amino-1-(4-chlorophenyl)-1-(6-(3-phenyl-1-tosyl-1H-pyrrolo[2,3-b]pyridin-4-yl)pyridin-3-yl)ethan-1-ol (0.070 g, 0.12 mmol) in ethanol (1 ml) was added 4 N aqueous sodium hydroxide (1.0 ml) at room temperature and the reaction mixture was heated at 70° C. for 10 min. The reaction mixture was concentrated, diluted with water, and extracted with ethyl acetate. The organic layer was dried with sodium sulfate and concentrated under reduced pressure. Purification by reverse phase chromatography (5% to 100% acetonitrile with 0.1% formic acid) gave the title product. ¹H NMR (400 MHz, DMSO-d₆) δ 12.05 (s, 1H), 8.48 (s, 1H), 8.34 (d, J=4.8 Hz, 1H), 8.24 (s, 1H), 7.62 (s, 1H), 7.56-7.41 (m, 5H), 7.18 (d, J=4.8 Hz, 1H), 6.99 (d, J=8.2 Hz, 1H), 6.90 (t, J=7.1 Hz, 1H), 6.82 (t, J=7.4 Hz, 2H), 6.76 (d, J=7.4 Hz, 2H), 3.48-3.14 (m, 2H). LCMS (m/z [M+H]⁺): 441.8

### Synthesis of Compound 1013 and Intermediates Thereof

Compound 1013 can be prepared as illustrated in Scheme 9 below.

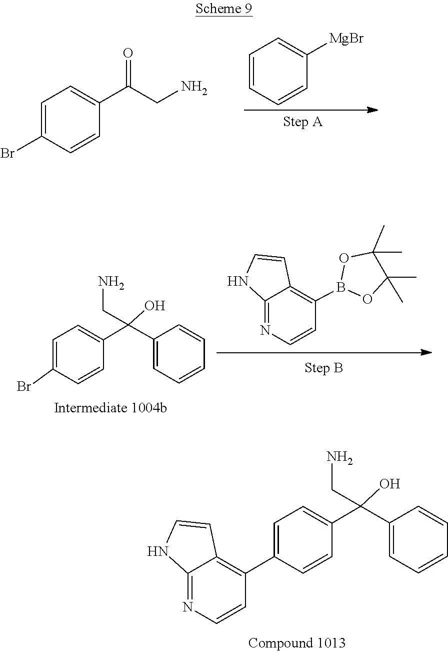

### Intermediate 1004b (Scheme9): 2-amino-1-(4-bromophenyl)-1-phenylethan-1-ol

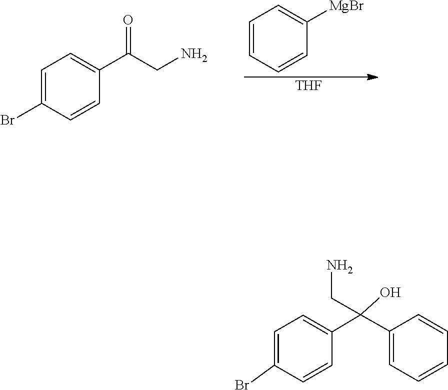

Step A: To a 20 mL microwave vial was added 2-amino-1-(4-bromophenyl)ethan-1-one (0.300 grams, 1.401 mmol) and a stirbar. The vial was sealed, and tetrahydrofuran (5 ml) was added via syringe. The vial was cooled to 0° C. with a water-ice bath, and phenymagenesium bromide in diethyl ether (7.0 mL, 1.0 M, 7.0 mmol) was added via syringe. The reaction was then heated in a microwave to 50° C. for 2 hours and stirred. The reaction was cooled, and poured into a solution of saturated aqueous sodium bicarbonate. The reaction mixture was then extracted with ethyl acetate, the organic phase was separated, and the solvent was removed by rotary evaporation. The crude product was purified by silica gel chromatography (0% to 15% methanol in dichloromethane) to give the title product. LCMS (m/z [M+H]⁺): 293.2

### Compound 1013

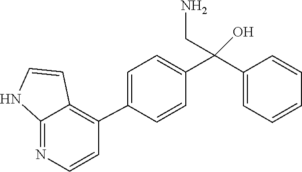 [SMILES: NCC(O)(c1ccccc1)c1ccc(-c2ccnc3[nH]ccc23)cc1]

### 1-(4-(1H-pyrrolo[2,3-b]pyridin-4-yl)phenyl)-2-amino-1-phenylethan-1-ol

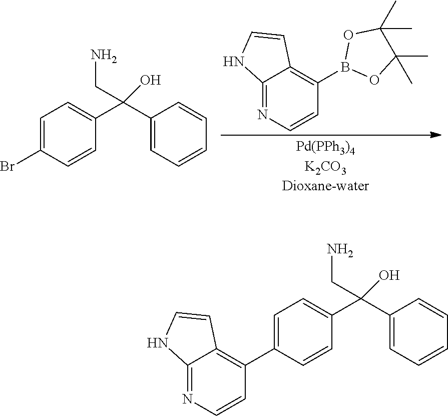 [SMILES: NCC(O)(c1ccccc1)c1ccc(Br)cc1 ; CC1(C)OB(c2ccnc3[nH]ccc23)OC1(C)C ; NCC(O)(c1ccccc1)c1ccc(-c2ccnc3[nH]ccc23)cc1]

Step B: To a 2 mL microwave vial containing a stirbar was added: 2-amino-1-(4-bromophenyl)-1-phenylethan-1-ol (intermediate 1004b) (0.050 grams, 0.171 mmol), followed by 4-(4,4,5,5-tetramethyl-1,3,2-dioxaborolan-2-yl)-1H-pyrrolo[2,3-b]pyridine (0.050 grams, 0.205 mmol), potassium carbonate (0.100 grams, 0.724 mmol) and tetrakis(triphenylphosphine)-palladium(0) (0.010 grams, 0.009 mmol). The vial was sealed, and 1,4-dioxane (1.5 ml) followed by water (0.5 ml) was added. The reaction mixture had nitrogen gas bubbled through it for 5 minutes at room temperature. It was then heated by microwave irradiation to 150° C. for 30 minutes. The reaction was cooled and poured into aqueous saturated sodium bicarbonate. It was extracted into ethyl acetate and the organic phase was separated and the solvent removed by rotary evaporation to give the crude product. Purification by reverse phase chromatography (5% to 100% acetonitrile in water with 0.1% trifluoroaceitic acid) gave the title product. ¹H NMR (400 MHz, DMSO-d₆) δ 11.84 (s, 1H), 8.28 (d, J=5.0 Hz, 1H), 7.89-7.74 (m, 5H), 7.74-7.64 (m, 2H), 7.64-7.46 (m, 3H), 7.39 (dd, J=8.5, 6.9 Hz, 2H), 7.34-7.24 (m, 1H), 7.19 (d, J=5.0 Hz, 1H), 6.66-6.47 (m, 2H), 3.76 (tq, J=12.8, 6.3 Hz, 2H). LCMS (m/z [M+H]⁺): 330.2

### Synthesis of Compound 1014 and Intermediates Thereof

Compound 1014 can be prepared as illustrated in Scheme 10 below.

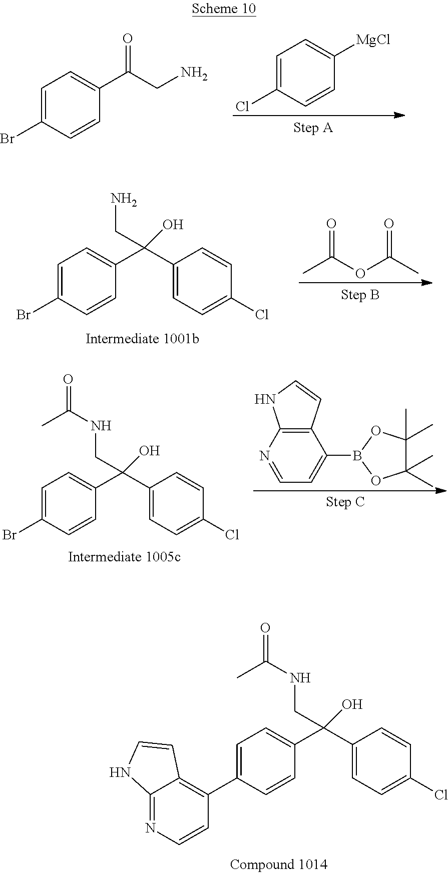

### Intermediate 1005c: N-(2-(4-bromophenyl)-2-(4-chlorophenyl)-2-hydroxyethyl)acetamide

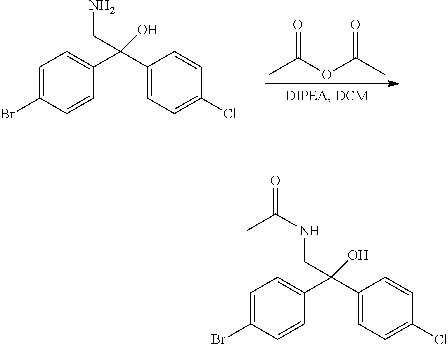

Step B: To a solution of 2-amino-1-(4-bromophenyl)-1-(4-chlorophenyl)ethan-1-ol (Intermediate 1001b, See Scheme 6, step A), (0.100 grams, 0.306 mmol) in dichloromethane (5.0 ml) was added acetic anhydride (0.031 g, 0.306 mmol), followed by diisopropylethylamine (0.059 g, 0.459 mmol, 0.080 ml). The reaction was stirred for 2 hours at room temperature. The reaction was then poured into an aqueous saturated sodium bicarbonate solution, and extracted into dichloromethane. The organic phase was separated, and the solvent removed by rotary evaporation. Purification by silica gel chromatography (0% to 100% ethyl acetate in hexanes) gave the title product. LCMS (m/z [M−OH+H]⁺): 352.0

### Compound 1014

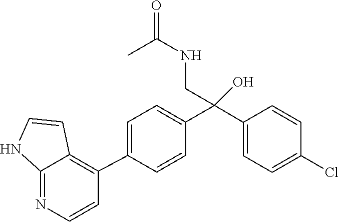 [SMILES: CC(=O)NCC(O)(c1ccc(Cl)cc1)c1ccc(-c2ccnc3[nH]ccc23)cc1]

### N-(2-(4-(1H-pyrrolo[2,3-b]pyridin-4-yl)phenyl)-2-(4-chlorophenyl)-2-hydroxyethyl)acetamide

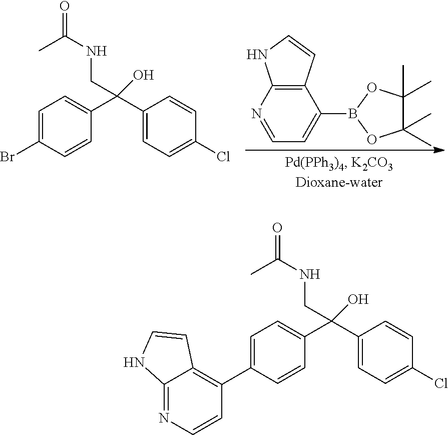 [SMILES: CC(=O)NCC(O)(c1ccc(Cl)cc1)c1ccc(Br)cc1 ; CC1(C)OB(c2ccnc3[nH]ccc23)OC1(C)C ; CC(=O)NCC(O)(c1ccc(Cl)cc1)c1ccc(-c2ccnc3[nH]ccc23)cc1]

Step C: To a 2 mL microwave vial containing a stirbar was added: 2 N-(2-(4-bromophenyl)-2-(4-chlorophenyl)-2-hydroxyethyl)acetamide (intermediate 1005c) (0.040 grams, 0.109 mmol), followed by 4-(4,4,5,5-tetramethyl-1,3,2-dioxaborolan-2-yl)-1H-pyrrolo[2,3-b]pyridine (0.026 grams, 0.109 mmol), potassium carbonate (0.03 grams, 0.217 mmol) and tetrakis(triphenylphosphine)-palladium(0) (0.013 grams, 0.011 mmol). The vial was sealed, and 1,4-dioxane (1.5 ml) followed by water (0.5 ml) was added. The reaction mixture had nitrogen gas bubbled through it for 5 minutes at room temperature. It was then heated by microwave irradiation to 150° C. for 30 minutes. The reaction was cooled and the solvent was removed by rotary evaporation. The crude product was dissolved in methanol (˜1 ml) and filtered. Purification by reverse phase chromatography (5% to 100% acetonitrile in water with 0.1% trifluoroacetic acid) gave the title product. ¹H NMR (400 MHz, DMSO-d₆) δ 11.88 (s, 1H), 8.29 (d, J=5.1 Hz, 1H), 7.79 (t, J=5.5 Hz, 1H), 7.73 (d, J=8.1 Hz, 2H), 7.60 (d, J=8.1 Hz, 2H), 7.55 (d, J=3.1 Hz, 1H), 7.50 (d, J=8.3 Hz, 2H), 7.37 (d, J=8.3 Hz, 2H), 7.21 (d, J=5.1 Hz, 1H), 6.63 (d, J=3.3 Hz, 1H), 4.00 (dd, J=13.8, 5.8 Hz, 1H), 3.87 (dd, J=13.7, 5.2 Hz, 1H), 1.77 (s, 3H). LCMS (m/z [M+H]⁺): 406.1

### Synthesis of Compounds of General Formula X1

Compounds of general formula X1 can be prepared as illustrated in Scheme 11 below.

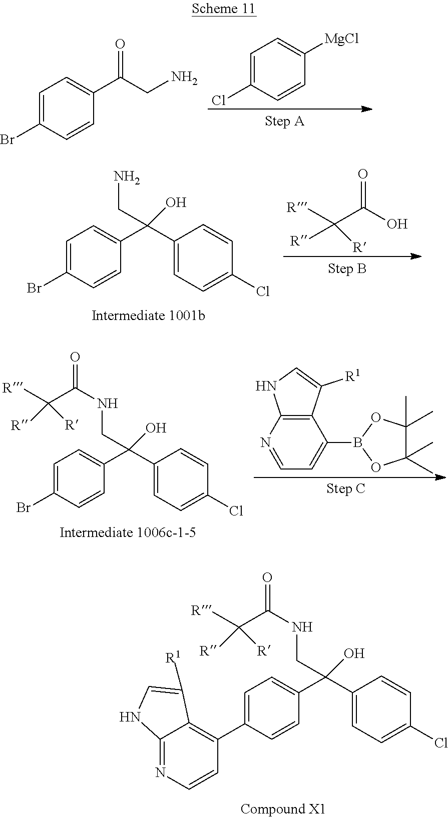

### Intermediate 1006c-1 (Scheme 11): N-(2-(4-bromophenyl)-2-(4-chlorophenyl)-2-hydroxyethyl)-2-hydroxyacetamide

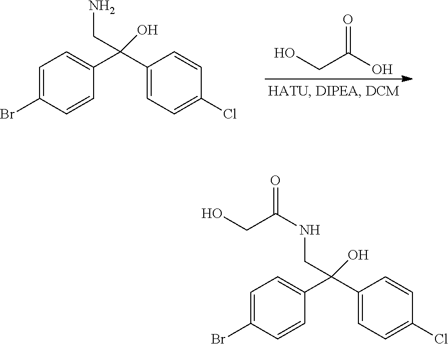 [SMILES: NCC(O)(c1ccc(Cl)cc1)c1ccc(Br)cc1 ; O=C(O)CO ; O=C(CO)NCC(O)(c1ccc(Cl)cc1)c1ccc(Br)cc1]

Step B: To a solution of 2-amino-1-(4-bromophenyl)-1-(4-chlorophenyl)ethan-1-ol (Intermediate 1001b, See Scheme 6, Step A), (0.100 grams, 0.306 mmol) in dichloromethane (5.0 ml) was added 2-hydroxyacetic acid (0.023 g, 0.306 mmol), followed by diisopropylethylamine (0.074 g, 0.573 mmol, 0.100 ml) and then 1-[bis(dimethylamino)methylene]-1H-1,2,3-triazolo[4,5-b]pyridinium 3-oxide hexafluorophosphate (0.116 g, 0.306 mmol). The reaction was stirred for 2 hours at room temperature. The reaction was then poured into an aqueous saturated sodium bicarbonate solution, and extracted into dichloromethane. The organic phase was separated, and the solvent removed by rotary evaporation. Purification by silica gel chromatography (0% to 100% ethyl acetate in hexanes) gave the title product. LCMS (m/z [M+Na]⁺): 408.0

### Intermediate 1006c-2 (Scheme 11): N-(2-(4-bromophenyl)-2-(4-chlorophenyl)-2-hydroxyethyl)-2-hydroxy-2-methylpropanamide

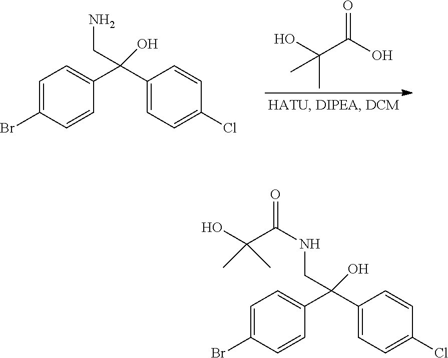

Step B: To a solution of 2-amino-1-(4-bromophenyl)-1-(4-chlorophenyl)ethan-1-ol (Intermediate 1001b, See Scheme 6, Step A), (0.100 grams, 0.306 mmol) in dichloromethane (5.0 ml) was added 2-hydroxy-2-methylpropanoic acid (0.05 g, 0.480 mmol), followed by diisopropylethylamine (0.074 g, 0.573 mmol, 0.100 ml) and then 1-[bis(dimethylamino)methylene]-1H-1,2,3-triazolo[4,5-b]pyridinium 3-oxide hexafluorophosphate (0.150 g, 0.394 mmol). The reaction was stirred for 2 hours at room temperature. The reaction was then poured into an aqueous saturated sodium bicarbonate solution, and extracted into dichloromethane. The organic phase was separated, and the solvent removed by rotary evaporation. Purification by silica gel chromatography (0% to 100% ethyl acetate in hexanes) gave the title product. LCMS (m/z [M+Na]⁺): 435.0

### Intermediate 1006c-3 (Scheme 11): N-(2-(4-bromophenyl)-2-(4-chlorophenyl)-2-hydroxyethyl)-2-(dimethylamino)acetamide

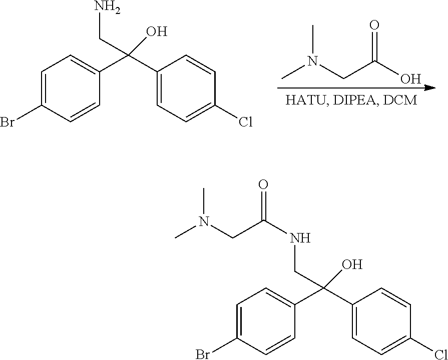

Step B: To a solution of 2-amino-1-(4-bromophenyl)-1-(4-chlorophenyl)ethan-1-ol (Intermediate 1001b, See Scheme 6, Step A), (0.100 grams, 0.306 mmol) in dichloromethane (5.0 ml) was added dimethylglycine (0.05 g, 0.485 mmol), followed by diisopropylethylamine (0.074 g, 0.573 mmol, 0.100 ml) and then 1-[bis(dimethylamino)methylene]-1H-1,2,3-triazolo[4,5-b]pyridinium 3-oxide hexafluorophosphate (0.130 g, 0.342 mmol). The reaction was stirred for 2 hours at room temperature. The reaction was then poured into an aqueous saturated sodium bicarbonate solution, and extracted into dichloromethane. The organic phase was separated, and the solvent removed by rotary evaporation. Purification by silica gel chromatography (0% to 100% ethyl acetate in hexanes) gave the title product. LCMS (m/z [M+H]⁺): 412.7

### Intermediate 1006c-4 (Scheme 11): tert-butyl (2-((2-(4-bromophenyl)-2-(4-chlorophenyl)-2-hydroxyethyl)amino)-2-oxoethyl)carbamate

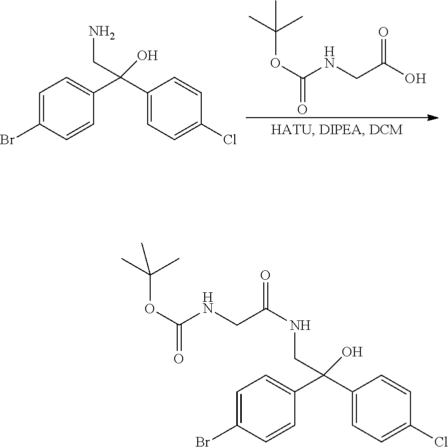 [SMILES: NCC(O)(c1ccc(Cl)cc1)c1ccc(Br)cc1 ; CC(C)(C)OC(=O)NCC(=O)O ; CC(C)(C)OC(=O)NCC(=O)NCC(O)(c1ccc(Cl)cc1)c1ccc(Br)cc1]

Step B: To a solution of 2-amino-1-(4-bromophenyl)-1-(4-chlorophenyl)ethan-1-ol (Intermediate 1001b, See Scheme 6, Step A), (0.100 grams, 0.306 mmol) in dichloromethane (5.0 ml) was added (tert-butylcarbonyl)glycine (0.045 g, 0.306 mmol), followed by diisopropylethylamine (0.074 g, 0.573 mmol, 0.100 ml) and then 1-[bis(dimethylamino)methylene]-1H-1,2,3-triazolo[4,5-b]pyridinium 3-oxide hexafluorophosphate (0.116 g, 0.306 mmol). The reaction was stirred for 2 hours at room temperature. The reaction was then poured into an aqueous saturated sodium bicarbonate solution, and extracted into dichloromethane. The organic phase was separated, and the solvent removed by rotary evaporation. Purification by silica gel chromatography (0% to 100% ethyl acetate in hexanes) gave the title product. LCMS (m/z [M+Na]⁺): 506.1

### Intermediate 1006c-5 (Scheme 11): (2-((2-(4-bromophenyl)-2-(4-chlorophenyl)-2-hydroxyethyl)amino)-2-oxoethyl)phosphonic acid

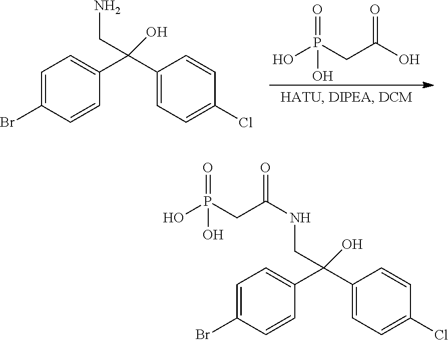 [SMILES: NCC(O)(c1ccc(Cl)cc1)c1ccc(Br)cc1 ; O=C(O)CP(=O)(O)O ; O=C(CP(=O)(O)O)NCC(O)(c1ccc(Cl)cc1)c1ccc(Br)cc1]

Step B: To a solution of 2-amino-1-(4-bromophenyl)-1-(4-chlorophenyl)ethan-1-ol (Intermediate 1001b, See Scheme 6, Step A), (0.100 grams, 0.306 mmol) in dichloromethane (5.0 ml) was added 2-phosphonoacetic acid (0.043 g, 0.306 mmol), followed by diisopropylethylamine (0.074 g, 0.573 mmol, 0.100 ml) and then 1-[bis(dimethylamino)methylene]-1H-1,2,3-triazolo[4,5-b]pyridinium 3-oxide hexafluorophosphate (0.116 g, 0.306 mmol). The reaction was stirred for 3 hours at room temperature. Trifluoroacetic acid (0.2 ml) was then added. The reaction solvent was removed by rotary evaporation to give the crude reaction mixture. Purification by silica gel chromatography (0% to 20% methanol acetate in dichloromethane) gave the title product with slight impurities which were directly moved on to the next step. LCMS (m/z [M−OH]⁺): 431.0

### Compound 1015

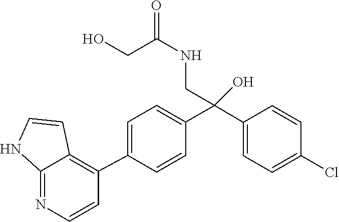 [SMILES: O=C(CO)NCC(O)(c1ccc(Cl)cc1)c1ccc(-c2ccnc3[nH]ccc23)cc1]

### N-(2-(4-(1H-pyrrolo[2,3-b]pyridin-4-yl)phenyl)-2-(4-chlorophenyl)-2-hydroxyethyl)-2-hydroxyacetamide

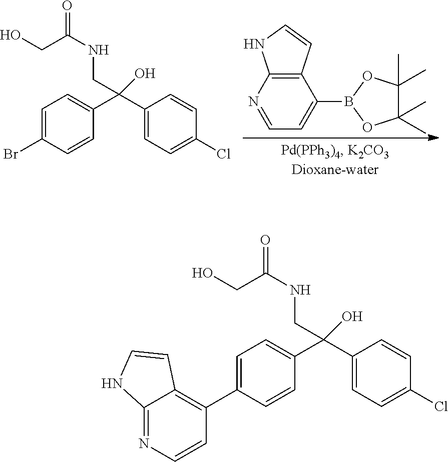 [SMILES: O=C(CO)NCC(O)(c1ccc(Cl)cc1)c1ccc(Br)cc1 ; CC1(C)OB(c2ccnc3[nH]ccc23)OC1(C)C ; O=C(CO)NCC(O)(c1ccc(Cl)cc1)c1ccc(-c2ccnc3[nH]ccc23)cc1]

Step C: To a 2 mL microwave vial containing a stirbar was added: 2 N-(2-(4-bromophenyl)-2-(4-chlorophenyl)-2-hydroxyethyl)acetamide (intermediate 1006c-1) (0.050 grams, 0.130 mmol), followed by 4-(4,4,5,5-tetramethyl-1,3,2-dioxaborolan-2-yl)-1H-pyrrolo[2,3-b]pyridine (0.050 grams, 0.205 mmol), potassium carbonate (0.100 grams, 0.724 mmol) and tetrakis(triphenylphosphine)-palladium(0) (0.015 grams, 0.013 mmol). The vial was sealed, and 1,4-dioxane (1.5 ml) followed by water (0.5 ml) was added. The reaction mixture had nitrogen gas bubbled through it for 5 minutes at room temperature. It was then heated by microwave irradiation to 150° C. for 30 minutes. The reaction was cooled and the solvent was removed by rotary evaporation. The crude product was dissolved in methanol (˜1 ml) and filtered. Purification by reverse phase chromatography (5% to 100% acetonitrile in water with 0.1% trifluoroacetic acid) gave the title product. ¹H NMR (400 MHz, DMSO-d₆) δ 11.88 (s, 1H), 8.29 (d, J=5.1 Hz, 1H), 7.74 (d, J=8.2 Hz, 2H), 7.61 (d, J=8.2 Hz, 2H), 7.52 (q, J=9.9, 8.7 Hz, 4H), 7.38 (d, J=8.4 Hz, 2H), 7.21 (d, J=5.1 Hz, 1H), 6.63 (s, 1H), 6.45 (s, 1H), 4.07 (dd, J=13.8, 5.9 Hz, 1H), 3.94 (dd, J=13.7, 5.1 Hz, 1H), 3.74 (s, 2H). LCMS (m/z [M+H]⁺): 422.1

### Compounds 1016-1022

Compounds 1016-1022 were prepared using an analogous procedure to that of Compound 1015 and are shown in Table 30.

TABLE 30

| | | | | LCMS |
|---|---|---|---|---|
| Compound | Structure | IUPAC name | ¹H NMR | m/z |
| 1016 | 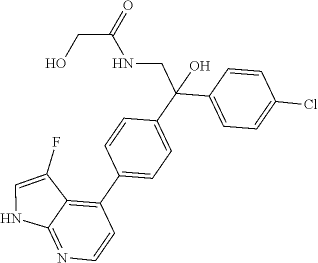 [SMILES: O=C(CO)NCC(O)(c1ccc(Cl)cc1)c1ccc(-c2ccnc3[nH]cc(F)c23)cc1] | N-(2-(4- chlorophenyl)-2- (4-(3-fluoro-1H- pyrrolo[2,3- b]pyridin-4- yl)phenyl)-2- hydroxyethyl)-2- hydroxyacetamide | ¹H NMR (400 MHz, DMSO-d₆) δ 11.64 (s, 1 H), 8.30 (d, J = 4.9 Hz, 1 H), 7.69 − 7.56 (m, 4 H), 7.50 (dd, J = 12.8, 6.8 Hz, 4 H), 7.38 (d, J = 8.3 Hz, 2 H), 7.13 (d, J = 5.0 Hz, 1 H), 4.08 (dd, J = 13.7, 6.0 Hz, 1 H), 3.93 (dd, J = 13.6, 5.0 Hz, 1 H), 3.74 (s, 2 H). | LCMS (m/z [M + H]⁺): 440.1 |
| 1017 | 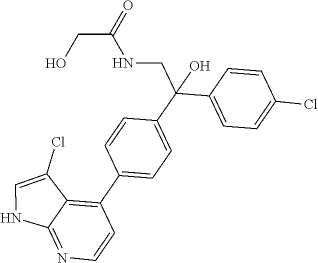 [SMILES: O=C(CO)NCC(O)(c1ccc(Cl)cc1)c1ccc(-c2ccnc3[nH]cc(Cl)c23)cc1] | N-(2-(4-(3-chloro- 1H-pyrrolo[2,3- b]pyridin-4- y1)phenyl)-2-(4- chlorophenyl)-2- hydroxyethyl)-2- hydroxyacetamide | ¹H NMR (400 MHz, DMSO-d₆) δ 12.15 (s, 1 H), 8.30 (d, J = 4.8 Hz, 1 H), 7.69 (d, J = 2.7 Hz, 1 H), 7.59 − 7.43 (m, 8H), 7.43 − 7.34 (m, 2 H), 7.02 (d, J = 4.9 Hz, 1 H), 4.06 (dd, J = 13.7, 5.9 Hz, 1 H), 3.93 (dd, J = 13.9, 5.2 Hz, 1 H), 3.73 (s, 2 H). | LCMS (m/z [M + H]⁺): 456.1 |
| 1018 | 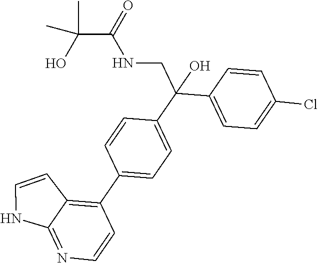 [SMILES: CC(C)(O)C(=O)NCC(O)(c1ccc(Cl)cc1)c1ccc(-c2ccnc3[nH]ccc23)cc1] | N-(2-(4-(1H- pyrrolo[2,3- b]pyridin-4- yl)phenyl)-2-(4- chlorophenyl)-2- hydroxyethyl)-2- hydroxy-2- methylpropanamide | ¹H NMR (400 MHz, DMSO-d₆) δ 11.89 (s, 1 H), 8.29 (d, J = 5.1 Hz, 1 H), 7.73 (d, J = 8.1 Hz, 2 H), 7.57 (dd, J = 15.2, 7.2 Hz, 4 H), 7.49 (d, J = 8.4 Hz, 2 H), 7.38 (d, J = 8.3 Hz, 2 H), 7.20 (d, J = 5.1 Hz, 1 H), 6.61 (d, J = 3.3 Hz, 1 H), 6.46 (s, 1 H), 3.99 (dd, J = 13.7, 5.7 Hz, 1 H), 3.92 (dd, J = 13.6, 5.4 Hz, 1 H), 1.07 (d, J = 7.2 Hz, 6 H). | LCMS (m/z [M + H]⁺): 450.2 |
| 1019 | 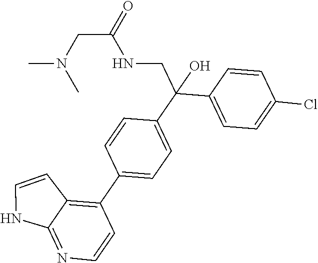 [SMILES: CN(C)CC(=O)NCC(O)(c1ccc(Cl)cc1)c1ccc(-c2ccnc3[nH]ccc23)cc1] | N-(2-(4-(1H- pyrrolo[2,3- b]pyridin-4- yl)phenyl)-2-(4- chlorophenyl)-2- hydroxyethyl)-2- (dimethylamino) acetamide | ¹H NMR (400 MHz, DMSO-d₆) δ 11.82 (s, 1 H), 9.54 (s, 1 H), 8.55 (s, 1 H), 8.27 (d, J = 5.0 Hz, 1 H), 7.74 (d, J = 8.2 Hz, 2 H), 7.61 (d, J = 8.1 Hz, 2 H), 7.58 − 7.47 (m, 3 H), 7.39 (d, J = 8.3 Hz, 2 H), 7.16 (d, J = 5.0 Hz, 1 H), 6.59 (t, J = 2.7 Hz, 1 H), 6.25 (s, 1 H), 4.08 (qd, J = 13.6, 5.7 Hz, 2 H), 3.85 − 3.74 (m, 2 H), 2.60 (d, J = 4.7 Hz, 6 H). | LCMS (m/z [M + H]⁺): 449.2 |
| 1020 | 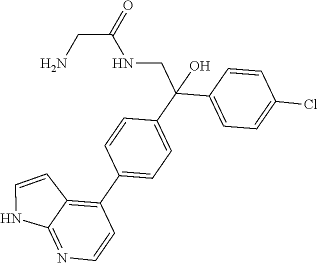 [SMILES: NCC(=O)NCC(O)(c1ccc(Cl)cc1)c1ccc(-c2ccnc3[nH]ccc23)cc1] | N-(2-(4-(1H- pyrrolo[2,3- b]pyridin-4- yl)phenyl)-2-(4- chlorophenyl)-2- hydroxyethyl)-2- aminoacetamide | ¹H NMR (400 MHz, DMSO-d₆) δ 11.82 (s, 1 H), 8.28 (d, J = 5.0 Hz, 1 H), 8.24 (t, J = 5.4 Hz, 1 H), 7.89 (s, 3 H), 7.74 (d, J = 8.1 Hz, 2 H), 7.61 (d, J = 8.1 Hz, 2 H), 7.58 − 7.48 (m, 3 H), 7.39 (d, J = 8.4 Hz, 2 H), 7.17 (d, J = 5.0 Hz, 1 H), 6.60 (t, J = 2.8 Hz, 1 H), 6.21 (s, 1 H), 4.13 (dd, J = 13.7, 5.9 Hz, 1 H), 3.98 (dd, J = 13.6, 5.1 Hz, 1 H), 3.48 (t, J = 5.9 Hz, 2 H). | LCMS (m/z [M + H]⁺) : 421.2 |
| 1021 | 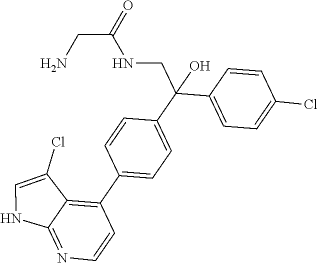 [SMILES: NCC(=O)NCC(O)(c1ccc(Cl)cc1)c1ccc(-c2ccnc3[nH]cc(Cl)c23)cc1] | 2-amino-N-(2-(4- (3-chloro-1H- pyrrolo[2,3- b]pyridin-4- yl)phenyl)-2-(4- chlorophenyl)-2- hydroxyethyl) acetamide | ¹H NMR (400 MHz, DMSO-d₆) δ 12.14 (d, J = 18.0 Hz, 1 H), 8.33 (dd, J = 17.2, 4.8 Hz, 1 H), 7.89 (s, 2 H), 7.67 (dd, J = 20.3, 2.7 Hz, 1 H), 7.38 (d, J = 8.4 Hz, 1 H), 7.04 (dd, J = 18.1, 4.8 Hz, 1 H), 6.22 (s, 1 H), 4.15 (dd, J = 13.7, 5.9 Hz, 1 H), 3.94 (dd, J = 13.7, 5.0 Hz, 1 H), 3.46 (d, J = 6.0 Hz, 2 H). | LCMS (m/z [M + H]⁺): 455.1 |
| 1022 | 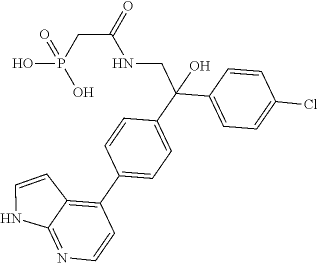 [SMILES: O=C(CP(=O)(O)O)NCC(O)(c1ccc(Cl)cc1)c1ccc(-c2ccnc3[nH]ccc23)cc1] | (2-((2-(4-(1H- pyrrolo[2,3- b]pyridin-4- yl)phenyl)-2-(4- chlorophenyl)-2- hydroxyethyl) amino)-2- oxoethyl) phosphonic acid | ¹H NMR (400 MHz, DMSO-d₆) δ 11.88 (s, 1 H), 8.30 (dd, J = 9.2, 5.0 Hz, 1 H), 7.98 − 7.68 (m, 3 H), 7.68 − 7.42 (m, 5 H), 7.36 − 7.13 (m, 2 H), 6.64 (dd, J = 3.5, 1.8 Hz, 1 H), 4.03 − 3.87 (m, 2 H), 2.77 − 2.53 (m, 2 H). | LCMS (m/z [M + H]⁺): 486.1 |

### Synthesis of Compounds of General Formula W3

Compounds of general formula W3 can be prepared as illustrated in Scheme 12 below.

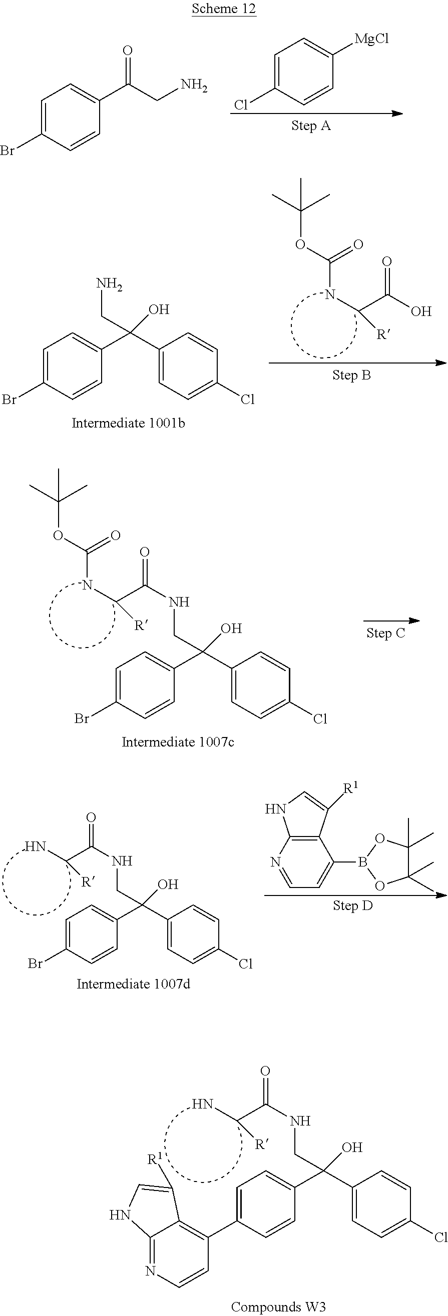

### Synthesis of Intermediates and Compounds of Scheme 12

### Intermediate 1007c-1 (Scheme 12): tert-butyl (2S)-2-((2-(4-bromophenyl)-2-(4-chlorophenyl)-2-hydroxyethyl)carbamoyl)pyrrolidine-1-carboxylate

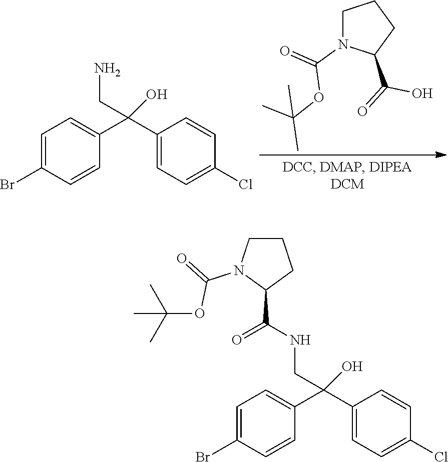 [SMILES: NCC(O)(c1ccc(Cl)cc1)c1ccc(Br)cc1 ; CC(C)(C)OC(=O)N1CCC[C@H]1C(=O)O ; CC(C)(C)OC(=O)N1CCC[C@H]1C(=O)NCC(O)(c1ccc(Cl)cc1)c1ccc(Br)cc1]

Step B: To a solution of 2-amino-1-(4-bromophenyl)-1-(4-chlorophenyl)ethan-1-ol (Intermediate 1001b, See Scheme 1, Step A), (0.200 grams, 0.612 mmol) in dichloromethane (10.0 ml) was added (tert-butyloxycarbonyl)-L-proline (0.132 g, 0.612 mmol), followed by diisopropylethylamine (0.119 g, 0.919 mmol, 0.160 ml), N,N′-dicyclohexylcarbodiimide (0.152 g, 0.735 mmol) and then 4-(dimethylamino)pyridine (0.152 g, 0.735 mmol). The reaction was stirred for 3 hours at room temperature. The reaction was then poured into an aqueous saturated sodium bicarbonate solution, and extracted into ethyl acetate. The organic phase was separated, and the solvent removed by rotary evaporation. Purification by silica gel chromatography (0% to 100% ethyl acetate in hexanes) gave the title product. Product was further characterized at the next step.

### Intermediate 1007d-1 (Scheme 12): (2S)—N-(2-(4-bromophenyl)-2-(4-chlorophenyl)-2-hydroxyethyl)pyrrolidine-2-carboxamide

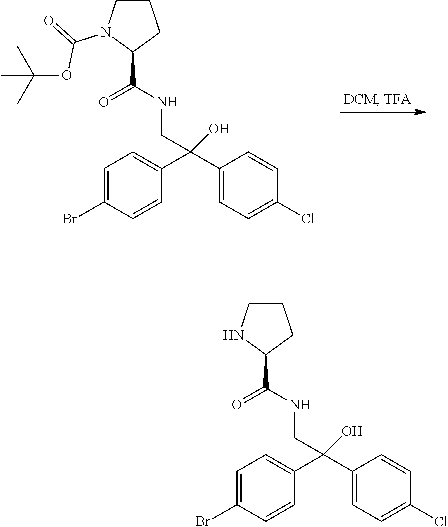 [SMILES: CC(C)(C)OC(=O)N1CCC[C@H]1C(=O)NCC(O)(c1ccc(Cl)cc1)c1ccc(Br)cc1 ; O=C(NCC(O)(c1ccc(Cl)cc1)c1ccc(Br)cc1)[C@@H]1CCCN1]

Step C: To a solution of tert-butyl (2S)-2-((2-(4-bromophenyl)-2-(4-chlorophenyl)-2-hydroxyethyl)carbamoyl)pyrrolidine-1-carboxylate (0.15 g, 0.286 mmol) in dichloromethane (2.0 ml) was added trifluoroacetic acid (2.96 g, 26.0 mmol, 2.0 mL). The reaction was stirred for 5 minutes at room temperature. The solvent and trifluoroacetic acid were then removed by rotary evaporation to give the crude product, which was used without further purification. LCMS (m/z [M+H]⁺): 424.7

### Intermediate 1007c-2 (Scheme 12): tert-butyl (2R)-2-((2-(4-bromophenyl)-2-(4-chlorophenyl)-2-hydroxyethyl)carbamoyl)pyrrolidine-1-carboxylate

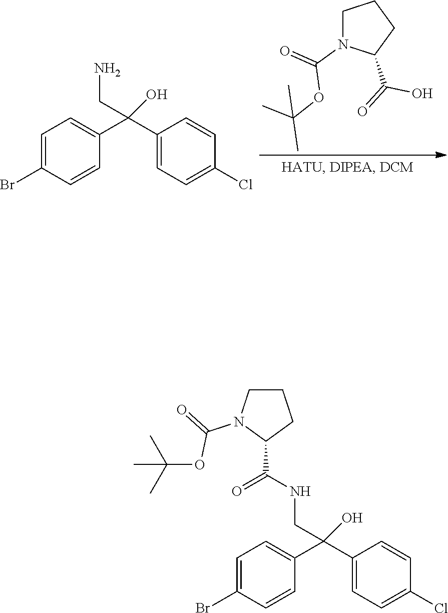 [SMILES: NCC(O)(c1ccc(Cl)cc1)c1ccc(Br)cc1 ; CC(C)(C)OC(=O)N1CCC[C@@H]1C(=O)O ; CC(C)(C)OC(=O)N1CCC[C@@H]1C(=O)NCC(O)(c1ccc(Cl)cc1)c1ccc(Br)cc1]

Step B: To a solution of 2-amino-1-(4-bromophenyl)-1-(4-chlorophenyl)ethan-1-ol (Intermediate 1001b, See Scheme 6, Step A), (0.100 grams, 0.306 mmol) in dichloromethane (3.0 ml) was added (tert-butoxycarbonyl)-D-proline (0.066 grams, 0.306 mmol, followed by 1-[bis(dimethylamino)methylene]-1H-1,2,3-triazolo[4,5-b]pyridinium 3-oxide hexafluorophosphate (0.116 g, 0.306 mmol) and diisopropylethylamine (0.073 g, 0.573 mmol, 0.1 ml). The reaction was stirred for 3 hours. It was then poured into a solution of saturated aqueous sodium bicarbonate. It was extracted with ethyl acetate, the organic phase was separated, and the solvent was removed by rotary evaporation. Purification by silica gel chromatography (0% to 100% ethyl acetate in hexanes) gave the desired product. LCMS (m/z [M+H]⁺): 525.1

### Intermediate 1007d-2 (Scheme 12): tert-butyl (2R)-2-((2-(4-bromophenyl)-2-(4-chlorophenyl)-2-hydroxyethyl)carbamoyl)pyrrolidine-1-carboxylate

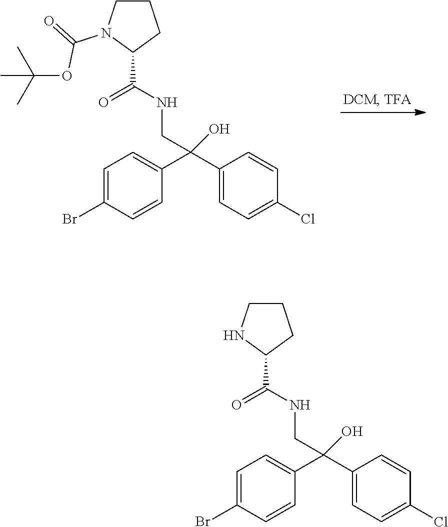 [SMILES: CC(C)(C)OC(=O)N1CCC[C@@H]1C(=O)NCC(O)(c1ccc(Cl)cc1)c1ccc(Br)cc1 ; O=C(NCC(O)(c1ccc(Cl)cc1)c1ccc(Br)cc1)[C@H]1CCCN1]

Step C: To a solution tert-butyl (2R)-2-((2-(4-bromophenyl)-2-(4-chlorophenyl)-2-hydroxyethyl)carbamoyl)pyrrolidine-1-carboxylate (0.080 g, 0.153 mmol) in dichloromethane (2.0 ml) was added trifluoroacetic acid (1.48 g, 13.0 mmol, 1.0 mL). The reaction was stirred for 15 minutes at room temperature. The solvent and trifluoroacetic acid were then removed by rotary evaporation to give the crude product, which was used without further purification. LCMS (m/z [M+H]⁺): 425.0

### Intermediate 1007c-3 (Scheme 12): tert-butyl (2-((2-((2-(4-bromophenyl)-2-(4-chlorophenyl)-2-hydroxyethyl)amino)-2-oxoethyl)amino)-2-oxoethyl)carbamate

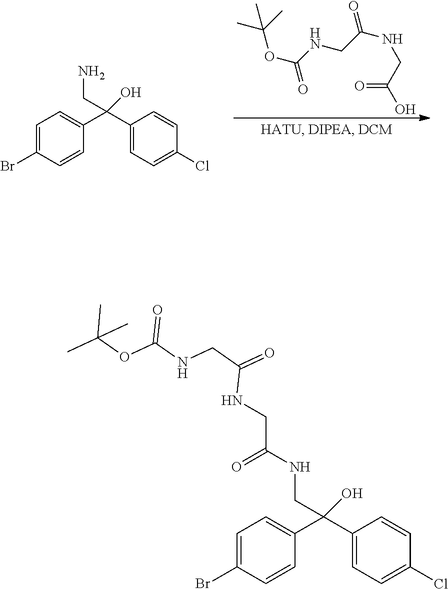 [SMILES: NCC(O)(c1ccc(Cl)cc1)c1ccc(Br)cc1 ; CC(C)(C)OC(=O)NCC(=O)NCC(=O)O ; CC(C)(C)OC(=O)NCC(=O)NCC(=O)NCC(O)(c1ccc(Cl)cc1)c1ccc(Br)cc1]

Step B: To a solution of 2-amino-1-(4-bromophenyl)-1-(4-chlorophenyl)ethan-1-ol (Intermediate 1001b, See Scheme 6, Step A), (0.100 grams, 0.306 mmol) in dichloromethane (3.0 ml) was added (tert-butoxycarbonyl)glycylglycine (0.071 grams, 0.306 mmol, followed by 1-[bis(dimethylamino)methylene]-1H-1,2,3-triazolo[4,5-b]pyridinium 3-oxide hexafluorophosphate (0.116 g, 0.306 mmol) and diisopropylethylamine (0.073 g, 0.573 mmol, 0.1 ml). The reaction was stirred for 3 hours. It was then poured into a solution of saturated aqueous sodium bicarbonate. It was extracted with ethyl acetate, the organic phase was separated, and the solvent was removed by rotary evaporation. Purification by silica gel chromatography (0% to 100% ethyl acetate in hexanes) gave the desired product. LCMS (m/z [M+Na]⁺): 561.6

### Intermediate 1007d-3 (Scheme 12): 2-amino-N-(2-((2-(4-bromophenyl)-2-(4-chlorophenyl)-2-hydroxyethyl)amino)-2-oxoethyl)acetamide

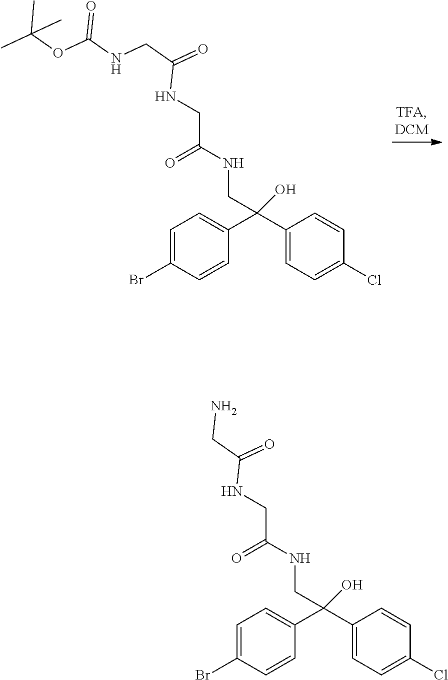 [SMILES: CC(C)(C)OC(=O)NCC(=O)NCC(=O)NCC(O)(c1ccc(Cl)cc1)c1ccc(Br)cc1 ; NCC(=O)NCC(=O)NCC(O)(c1ccc(Cl)cc1)c1ccc(Br)cc1]

Step C: To a solution of tert-butyl (2-((2-((2-(4-bromophenyl)-2-(4-chlorophenyl)-2-hydroxyethyl)amino)-2-oxoethyl)amino)-2-oxoethyl)carbamate (0.151 g, 0.280 mmol) in dichloromethane (2.0 ml) was added trifluoroacetic acid (2.29 g, 26.0 mmol, 2.0 mL). The reaction was stirred for 15 minutes at room temperature. The solvent and trifluoroacetic acid were then removed by rotary evaporation to give the crude product, which was used without further purification. LCMS (m/z [M+Na]⁺): 462.0

### Intermediate 1007c-4 (Scheme 12): tert-butyl ((2S)-1-((2-(4-bromophenyl)-2-(4-chlorophenyl)-2-hydroxyethyl)amino)-1-oxopropan-2-yl)carbamate

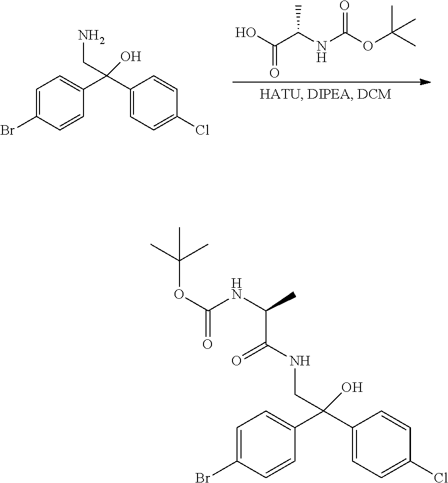 [SMILES: NCC(O)(c1ccc(Cl)cc1)c1ccc(Br)cc1 ; C[C@H](NC(=O)OC(C)(C)C)C(=O)O ; C[C@H](NC(=O)OC(C)(C)C)C(=O)NCC(O)(c1ccc(Cl)cc1)c1ccc(Br)cc1]

Step B: To a solution of 2-amino-1-(4-bromophenyl)-1-(4-chlorophenyl)ethan-1-ol (Intermediate 1001b, See Scheme 6, Step A), (0.150 grams, 0.459 mmol) in dichloromethane (5.0 ml) was added (tert-butyloxycarbonyl)-L-alanine (0.087 g, 0.459 mmol), followed by 1-[bis(dimethylamino)methylene]-1H-1,2,3-triazolo[4,5-b]pyridinium 3-oxide hexafluorophosphate (0.175 g, 0.459 mmol), then diisopropylethylamine (0.074 g, 0.573 mmol, 0.100 ml). The reaction was stirred for 3 hours at room temperature. The reaction was then poured into an aqueous saturated sodium bicarbonate solution, and extracted into ethyl acetate. The organic phase was separated, and the solvent removed by rotary evaporation. Purification by silica gel chromatography (0% to 100% ethyl acetate in hexanes) gave the title product. LCMS (m/z [M+Na]⁺): 521.0

### Intermediate 1007d-4 (Scheme 12): (2S)-2-amino-N-(2-(4-bromophenyl)-2-(4-chlorophenyl)-2-hydroxyethyl)propanamide

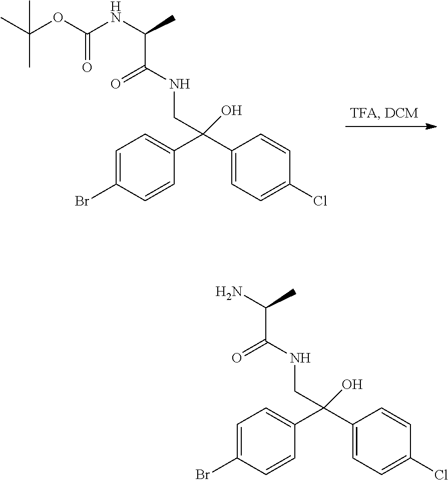 [SMILES: C[C@H](NC(=O)OC(C)(C)C)C(=O)NCC(O)(c1ccc(Cl)cc1)c1ccc(Br)cc1 ; C[C@H](N)C(=O)NCC(O)(c1ccc(Cl)cc1)c1ccc(Br)cc1]

Step C: To a solution of tert-butyl ((2S)-1-((2-(4-bromophenyl)-2-(4-chlorophenyl)-2-hydroxyethyl)amino)-1-oxopropan-2-yl)carbamate (0.085 g, 0.171 mmol) in dichloromethane (2.0 ml) was added trifluoroacetic acid (2.29 g, 26.0 mmol, 2.0 mL). The reaction was stirred for 30 minutes at room temperature. The solvent and trifluoroacetic acid were then removed by rotary evaporation to give the crude product, which was used without further purification. LCMS (m/z [M+H]⁺): 399.0 Intermediate 1007c-5 (Scheme 12): tert-butyl (2-((2-(4-bromophenyl)-2-(4-chlorophenyl)-2-hydroxyethyl)amino)-2-oxoethyl)(methyl)carbamate

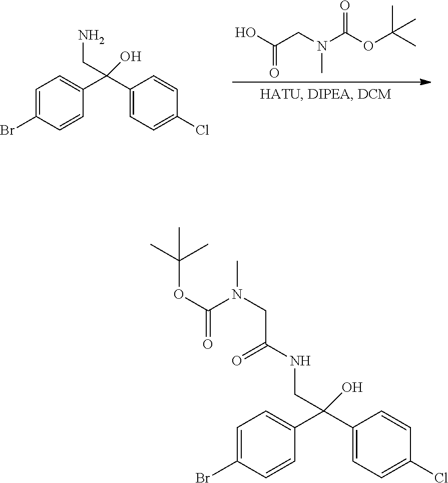 [SMILES: NCC(O)(c1ccc(Cl)cc1)c1ccc(Br)cc1 ; CN(CC(=O)O)C(=O)OC(C)(C)C ; CN(CC(=O)NCC(O)(c1ccc(Cl)cc1)c1ccc(Br)cc1)C(=O)OC(C)(C)C]

Step B: To a solution of 2-amino-1-(4-bromophenyl)-1-(4-chlorophenyl)ethan-1-ol (Intermediate 1001b, See Scheme 6, Step A), (0.100 grams, 0.306 mmol) in dichloromethane (5.0 ml) was added N-(tert-butyoxycarbonyl)-N-methylglycine (0.058 g, 0.306 mmol), followed by 1-[bis(dimethylamino)methylene]-1H-1,2,3-triazolo[4,5-b]pyridinium 3-oxide hexafluorophosphate (0.116 g, 0.306 mmol), then diisopropylethylamine (0.074 g, 0.573 mmol, 0.100 ml). The reaction was stirred for 3 hours at room temperature. The reaction was then poured into an aqueous saturated sodium bicarbonate solution, and extracted into ethyl acetate. The organic phase was separated, and the solvent removed by rotary evaporation. Purification by silica gel chromatography (0% to 100% ethyl acetate in hexanes) gave the title product. LCMS (m/z [M+Na]⁺): 521.0

### Intermediate 1007d-5 (Scheme 12): N-(2-(4-bromophenyl)-2-(4-chlorophenyl)-2-hydroxyethyl)-2-(methylamino)acetamide

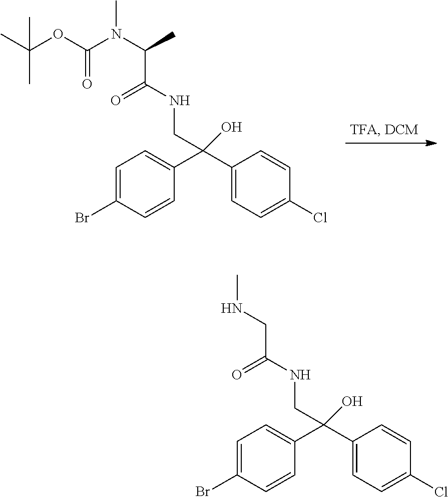 [SMILES: C[C@@H](C(=O)NCC(O)(c1ccc(Cl)cc1)c1ccc(Br)cc1)N(C)C(=O)OC(C)(C)C ; CNCC(=O)NCC(O)(c1ccc(Cl)cc1)c1ccc(Br)cc1]

Step C: To a solution of tert-butyl (2-((2-(4-bromophenyl)-2-(4-chlorophenyl)-2-hydroxyethyl)amino)-2-oxoethyl)(methyl)carbamate (0.105 g, 0.211 mmol) in dichloromethane (1.0 ml) was added trifluoroacetic acid (1.48 g, 13.0 mmol, 1.0 mL). The reaction was stirred for 30 minutes at room temperature. The solvent and trifluoroacetic acid were then removed by rotary evaporation to give the crude product. The crude product was treated with aqueous saturated sodium bicarbonate and extracted with ethyl acetate. The organic phase was separated, and the solvent was removed by rotary evaporation to give the product which was utilized without further purification. LCMS (m/z [M+H]⁺): 399.0

### Intermediate 1007c-6 (Scheme 12): tert-butyl (1-((2-(4-bromophenyl)-2-(4-chlorophenyl)-2-hydroxyethyl)amino)-2-methyl-1-oxopropan-2-yl)carbamate

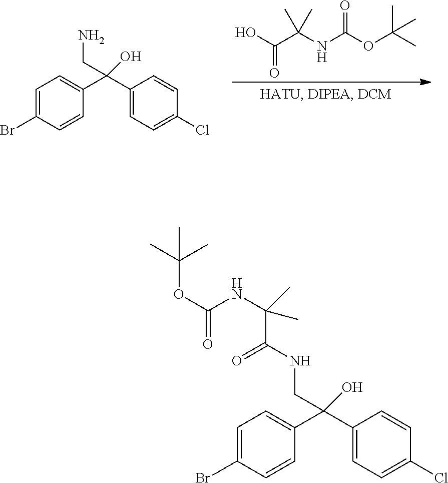 [SMILES: NCC(O)(c1ccc(Cl)cc1)c1ccc(Br)cc1 ; CC(C)(C)OC(=O)NC(C)(C)C(=O)O ; CC(C)(C)OC(=O)NC(C)(C)C(=O)NCC(O)(c1ccc(Cl)cc1)c1ccc(Br)cc1]

Step B: To a solution of 2-amino-1-(4-bromophenyl)-1-(4-chlorophenyl)ethan-1-ol (Intermediate 1001b, See Scheme 6, Step A), (0.150 grams, 0.459 mmol) in dichloromethane (5.0 ml) was added 2-((tert-butoxycarbonyl)amino)-2-methylpropanoic acid (0.093 grams, 0.459 mmol), followed by 1-[bis(dimethylamino)methylene]-1H-1,2,3-triazolo[4,5-b]pyridinium 3-oxide hexafluorophosphate (0.175 g, 0.459 mmol), then diisopropylethylamine (0.074 g, 0.573 mmol, 0.100 ml). The reaction was stirred for 3 hours at room temperature. The reaction was then poured into an aqueous saturated sodium bicarbonate solution, and extracted into ethyl acetate. The organic phase was separated, and the solvent removed by rotary evaporation. Purification by silica gel chromatography (0% to 100% ethyl acetate in hexanes) gave the title product. LCMS (m/z [M+H]⁺): 513.1

### Intermediate 1007d-6: 2-amino-N-(2-(4-bromophenyl)-2-(4-chlorophenyl)-2-hydroxyethyl)-2-methylpropanamide

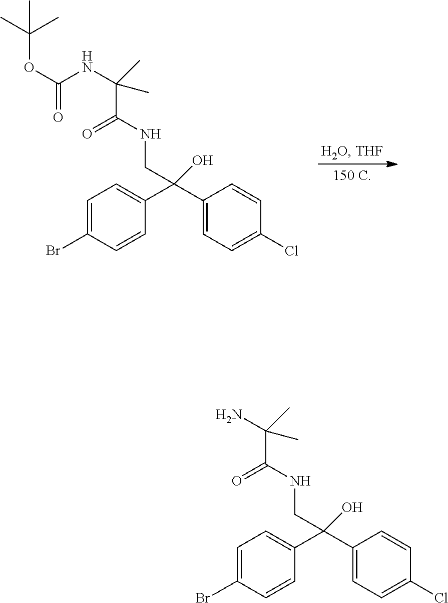

Step C: To a microwave vial with a stirbar was added tert-butyl (1-((2-(4-bromophenyl)-2-(4-chlorophenyl)-2-hydroxyethyl)amino)-2-methyl-1-oxopropan-2-yl)carbamate (0.105 g, 2.05 mmol). Tetrahydrofuran (2.0 mL) was added followed by water (2.0 ml), and the vial was sealed. The reaction was then heated to 150° C. under microwave irradiation for 30 minutes. The reaction was cooled, poured into aqueous saturated sodium bicarbonate and extracted into ethyl acetate. The organic phase was separated, and the solvent removed by rotary evaporation to give the title product, which was used without further purification. LCMS (m/z [M+H]⁺): 413.0

### Compound 1023

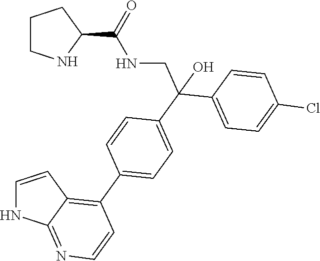 [SMILES: O=C(NCC(O)(c1ccc(Cl)cc1)c1ccc(-c2ccnc3[nH]ccc23)cc1)[C@@H]1CCCN1]

### (2S)—N-(2-(4-(1H-pyrrolo[2,3-b]pyridin-4-yl)phenyl)-2-(4-chlorophenyl)-2-hydroxyethyl)pyrrolidine-2-carboxamide

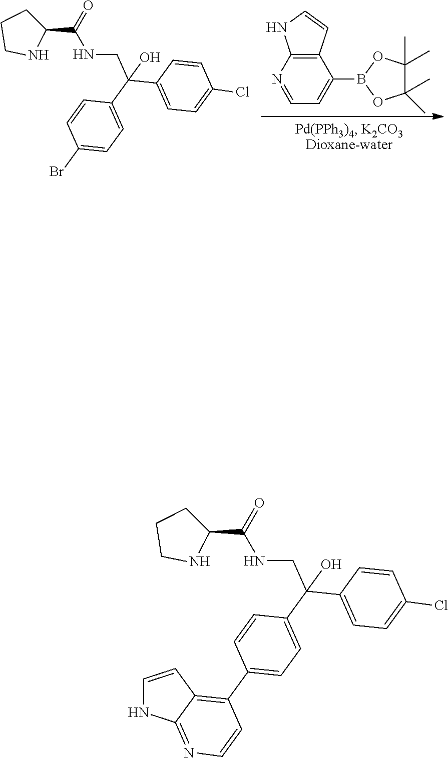

Step D: To a 2 mL microwave vial containing a stirbar was added (2S)—N-(2-(4-bromophenyl)-2-(4-chlorophenyl)-2-hydroxyethyl)pyrrolidine-2-carboxamide (intermediate 1007d-1) (0.050 grams, 0.118 mmol), followed by 4-(4,4,5,5-tetramethyl-1,3,2-dioxaborolan-2-yl)-1H-pyrrolo[2,3-b]pyridine (0.050 grams, 0.205 mmol), potassium carbonate (0.100 grams, 0.724 mmol) and tetrakis(triphenylphosphine)-palladium(0) (0.010 grams, 0.009 mmol). The vial was sealed, and 1,4-dioxane (1.5 ml) followed by water (0.5 ml) was added. The reaction mixture had nitrogen gas bubbled through it for 5 minutes at room temperature. It was then heated by microwave irradiation to 150° C. for 30 minutes. The reaction was cooled and poured into aqueous saturated sodium bicarbonate. The mixture was extracted with ethyl acetate, the organic phase was separated, and the solvent removed by rotary evaporation. The crude product was dissolved in methanol (˜1 ml) and filtered. Purification by reverse phase chromatography (5% to 100% acetonitrile in water with 0.1% trifluoroacetic acid) gave the title product. ¹H NMR (400 MHz, DMSO-d₆) δ 11.83 (s, 1H), 9.01 (s, 1H), 8.44 (t, J=12.1 Hz, 2H), 8.28 (dd, J=4.9, 3.4 Hz, 1H), 7.74 (dd, J=19.1, 8.2 Hz, 2H), 7.66-7.46 (m, 5H), 7.39 (dd, J=14.1, 8.4 Hz, 2H), 7.16 (dd, J=11.4, 5.0 Hz, 1H), 6.65-6.50 (m, 1H), 6.28 (s, 1H), 4.41 (ddd, J=21.6, 13.6, 7.5 Hz, 1H), 4.11 (d, J=7.0 Hz, 1H), 3.74 (t, J=15.5 Hz, 1H), 3.13 (d, J=34.4 Hz, 2H), 2.19-1.94 (m, 1H), 1.84-1.46 (m, 1H), 1.28 (dt, J=14.1, 7.1 Hz, 1H). LCMS (m/z [M+H]⁺). 461.2

### Compounds 1024-1028

Compounds 1024-1028 were prepared using an analogous procedure to that of Compound 1023 and are shown in Table 31.

TABLE 31

| | | | | LCMS |
|---|---|---|---|---|
| Compound | Structure | IUPAC name | ¹H NMR | m/z |
| 1024 | 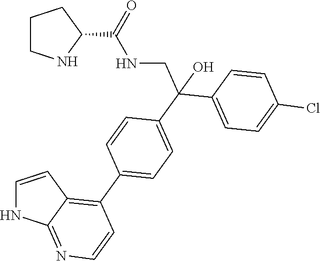 [SMILES: O=C(NCC(O)(c1ccc(Cl)cc1)c1ccc(-c2ccnc3[nH]ccc23)cc1)[C@H]1CCCN1] | (2R)-N-(2-(4-(1H- pyrrolo[2,3- b]pyridin-4- yl)phenyl)-2-(4- chlorophenyl)-2- hydroxyethyl) pyrrolidine-2- carboxamide | ¹H NMR (400 MHz, DMSO-d₆) δ 11.83 (s, 1 H), 9.01 (s, 1 H), 8.28 (dd, J = 5.0, 3.5 Hz, 1 H), 7.74 (dd, J = 19.0, 8.1 Hz, 2 H), 7.68 − 7.49 (m, 4 H), 7.49 − 7.34 (m, 2 H), 7.18 (td, J = 12.2, 11.4, 5.1 Hz, 1 H), 6.75 − 6.45 (m, 1 H), 6.28 (s, 1 H), 4.41 (ddd, J = 21.5, 13.4, 7.6 Hz, 1 H), 4.12 (d, J = 7.3 Hz, 1 H), 3.74 (t, J = 16.2 Hz, 1 H), 3.12 (d, J = 35.1 Hz, 2 H), | LCMS (m/z [M + H]⁺): 461.1 |
| | | | 2.01 (d, J = 11.6 Hz, 1 | |
| | | | H), 1.82 − 1.37 (m, 1 | |
| | | | H), 1.27 (dq, J = 13.2, | |
| | | | 6.4 Hz, 1 H). | |
| 1025 | 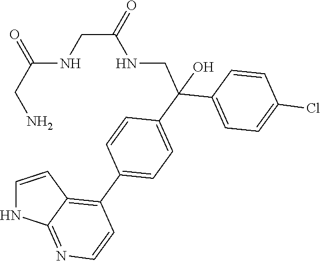 [SMILES: NCC(=O)NCC(=O)NCC(O)(c1ccc(Cl)cc1)c1ccc(-c2ccnc3[nH]ccc23)cc1] | N-(2-(4-(1H- pyrrolo[2,3- b]pyridin-4- yl)phenyl)-2-(4- chlorophenyl)-2- hydroxyethyl)-2-(2- aminoacetamido) acetamide | ¹H NMR (400 MHz, DMSO-d₆) δ 11.82 (s, 1 H), 8.49 (t, J = 5.5 Hz, 1 H), 8.27 (d, J = 4.9 Hz, 1 H), 7.93 (d, J = 15.6 Hz, 4 H), 7.73 (d, J = 8.1 Hz, 2 H), 7.59 (d, J = 8.0 Hz, 2 H), 7.54 (t, J = 3.1 Hz, 1 H), 7.49 (d, J = 8.4 Hz, 2 H), 7.38 (d, J = 8.4 Hz, 2 H), 7.17 (d, J = 5.0 Hz, 1 H), 6.61 (d, J = 3.4 Hz, 1 H), 6.18 (s, 1 H), 4.06 (dd, J = 13.8, 5.9 Hz, 1 H), 3.91 | LCMS (m/z [M + H]⁺): 478.2 |
| | | | (dd, J = 13.8, 5.1 Hz, 1 | |
| | | | H), 3.82 − 3.66 (m, 2 | |
| | | | H), 3.57 (d, J = 5.9 Hz, | |
| | | | 2 H), 1.18 (t, J = 7.3 | |
| | | | Hz, 1 H). | |
| 1026 | 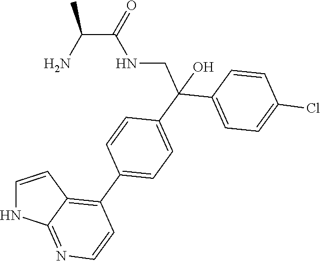 [SMILES: C[C@H](N)C(=O)NCC(O)(c1ccc(Cl)cc1)c1ccc(-c2ccnc3[nH]ccc23)cc1] | (2S)-N-(2-(4-(1H- pyrrolo[2,3- b]pyridin-4- yl)phenyl)-2-(4- chlorophenyl)-2- hydroxyethyl)-2- aminopropanamide | ¹H NMR (400 MHz, DMSO-d₆) δ 11.83 (s, 1 H), 8.28 (q, J = 5.1, 4.3 Hz, 2 H), 7.93 (s, 3 H), 7.76 (d, J = 8.2 Hz, 1 H), 7.70 (d, J = 8.1 Hz, 1 H), 7.62 (d, J = 8.0 Hz, 1 H), 7.55 (dd, J = 15.1, 8.3 Hz, 3 H), 7.48 (d, J = 8.4 Hz, 1 H), 7.41 (d, J = 8.5 Hz, 1 H), 7.36 (d, J = 8.4 Hz, 1 H), 7.16 (dd, J = 13.9, 5.1 Hz, 1 H), 6.58 (d, J = 17.1 Hz, 1 H), 6.26 (s, 1 H), 4.47 | LCMS (m/z [M + H]⁺): 435.2 |
| | | | (ddd, J = 29.8, 13.8, | |
| | | | 8.3 Hz, 1 H), 3.81 (s, 1 | |
| | | | H), 3.73 − 3.50 (m, 1 | |
| | | | H), 0.96 (t, J = 6.7 Hz, | |
| | | | 3 H). | |
| 1027 | 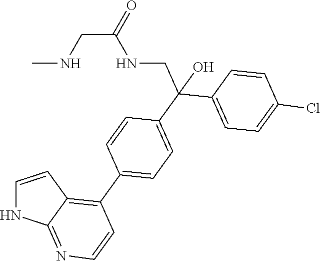 [SMILES: CNCC(=O)NCC(O)(c1ccc(Cl)cc1)c1ccc(-c2ccnc3[nH]ccc23)cc1] | N-(2-(4-(1H- pyrrolo[2,3- b]pyridin-4- yl)phenyl)-2-(4- chlorophenyl)-2- hydroxyethyl)-2- (methylamino) acetamide | ¹H NMR (400 MHz, DMSO-d₆) δ 11.83 (s, 1 H), 8.59 (s, 2 H), 8.37 (t, J = 5.6 Hz, 1 H), 8.28 (d, J = 5.0 Hz, 1 H), 7.74 (d, J = 8.1 Hz, 2 H), 7.61 (d, J = 8.1 Hz, 2 H), 7.58 − 7.46 (m, 3 H), 7.39 (d, J = 8.3 Hz, 2 H), 7.17 (d, J = 5.0 Hz, 1 H), 6.60 (s, 1 H), 6.23 (s, 1 H), 4.13 (dd, J = 13.6, 5.9 Hz, 1 H), 3.99 (dd, J = 13.4, 5.1 Hz, 1 H), 3.62 (s, 2 H), 2.42 (t, | LCMS (m/z [M + H]⁺): 435.2 |
| | | | J = 5.3 Hz, 3 H). | |
| 1028 | 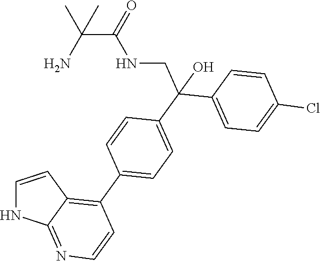 [SMILES: CC(C)(N)C(=O)NCC(O)(c1ccc(Cl)cc1)c1ccc(-c2ccnc3[nH]ccc23)cc1] | N-(2-(4-(1H- pyrrolo[2,3- b]pyridin-4- yl)phenyl)-2-(4- chlorophenyl)-2- hydroxyethyl)-2- amino-2- methylpropanamide | ¹H NMR (400 MHz, DMSO-d₆) δ 11.83 (s, 1 H), 8.28 (d, J = 5.0 Hz, 1 H), 8.03 (s, 3 H), 7.97 − 7.87 (m, 1 H), 7.74 (d, J = 8.1 Hz, 2 H), 7.64 − 7.52 (m, 3 H), 7.49 (d, J = 8.4 Hz, 2 H), 7.39 (d, J = 8.5 Hz, 2 H), 7.17 (d, J = 5.0 Hz, 1 H), 6.60 (dd, J = 3.9, 1.8 Hz, 1 H), 6.23 (s, 1 H), 4.22 − 3.91 (m, 2 H), 2.54 (s, 2 H), 1.25 (d, J = 8.2 Hz, 6 H). | LCMS (m/z [M + H]⁺): 449.2 |

### Synthesis of Compound 1029 and Intermediates Thereof

Compound 1029 can be prepared as illustrated in Scheme 13 below.

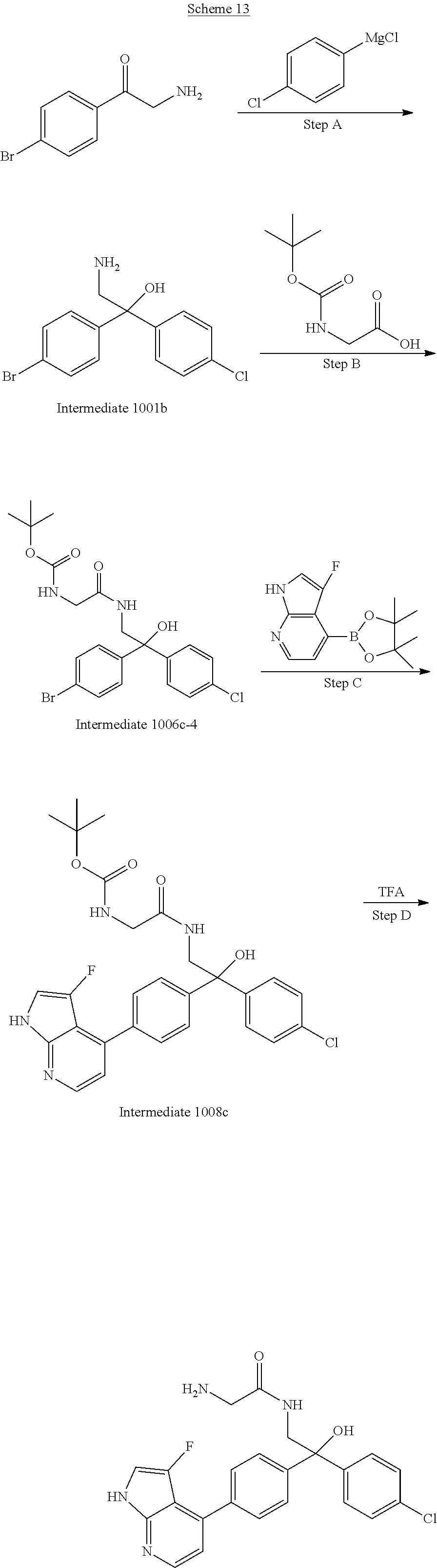

### Intermediate 1008d (Scheme 13): tert-butyl (2-((2-(4-chlorophenyl)-2-(4-(3-fluoro-1H-pyrrolo[2,3-b]pyridin-4-yl)phenyl)-2-hydroxyethyl)amino)-2-oxoethyl)carbamate

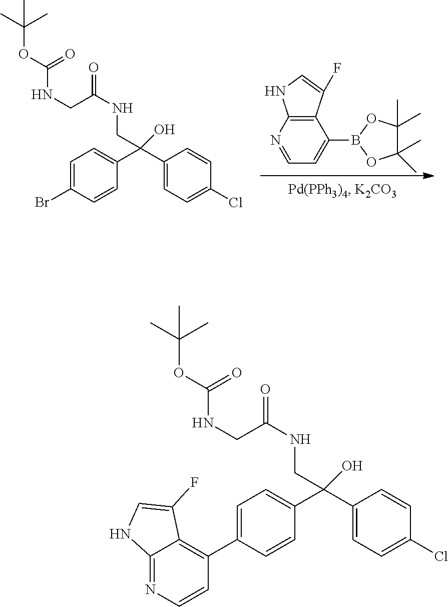

Step C: To a 2 mL microwave vial containing a stirbar was added tert-butyl (2-((2-(4-bromophenyl)-2-(4-chlorophenyl)-2-hydroxyethyl)amino)-2-oxoethyl)carbamate (intermediate 1006c-4) (0.050 grams, 0.103 mmol), followed by 3-fluoro-4-(4,4,5,5-tetramethyl-1,3,2-dioxaborolan-2-yl)-1H-pyrrolo[2,3-b]pyridine (0.030 grams, 0.114 mmol), potassium carbonate (0.100 grams, 0.724 mmol) and tetrakis(triphenylphosphine)-palladium(0) (0.010 grams, 0.009 mmol). The vial was sealed, and 1,4-dioxane (1.5 ml) followed by water (0.5 ml) was added. The reaction mixture had nitrogen gas bubbled through it for 5 minutes at room temperature. It was then heated by microwave irradiation to 150° C. for 30 minutes. The reaction was cooled and poured into aqueous saturated sodium bicarbonate. The mixture was extracted with ethyl acetate, the organic phase was separated, and the solvent removed by rotary evaporation to give the crude product, which was immediately utilized in the next reaction.

### Compound 1029

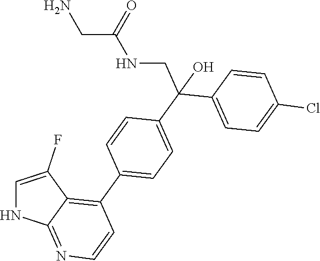 [SMILES: NCC(=O)NCC(O)(c1ccc(Cl)cc1)c1ccc(-c2ccnc3[nH]cc(F)c23)cc1]

### 2-amino-N-(2-(4-chlorophenyl)-2-(4-(3-fluoro-1H-pyrrolo[2,3-b]pyridin-4-yl)phenyl)-2-hydroxyethyl)acetamide

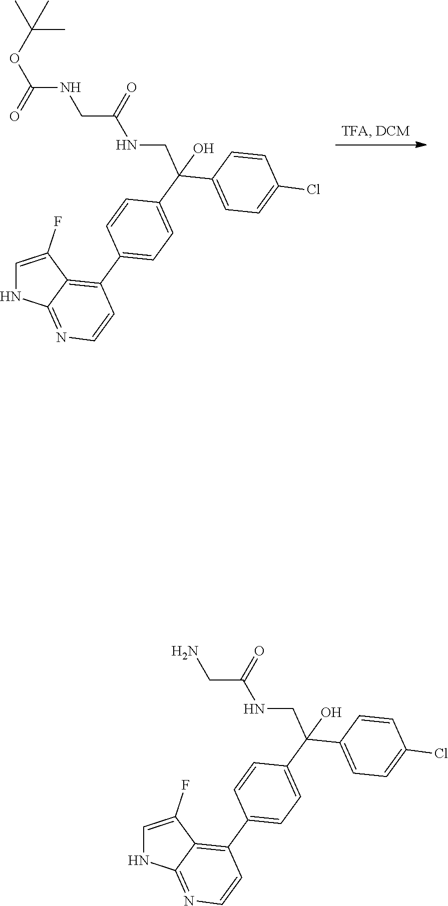 [SMILES: CC(C)(C)OC(=O)NCC(=O)NCC(O)(c1ccc(Cl)cc1)c1ccc(-c2ccnc3[nH]cc(F)c23)cc1 ; NCC(=O)NCC(O)(c1ccc(Cl)cc1)c1ccc(-c2ccnc3[nH]cc(F)c23)cc1]

Step D: Crude tert-butyl (2-((2-(4-chlorophenyl)-2-(4-(3-fluoro-1H-pyrrolo[2,3-b]pyridin-4-yl)phenyl)-2-hydroxyethyl)amino)-2-oxoethyl)carbamate (0.103 mmol, based on previous reaction) was placed in a vial. Dichloromethane (1.0 ml) and trifluoroacetic acid (1.0 ml) were added to the vial and the reaction was stirred at room temperature for 30 minutes. The solvent and trifluoroacetic acid were then removed by rotary evaporation. The crude product was dissolved in methanol (˜1 ml) and filtered. Purification by reverse phase chromatography (5% to 100% acetonitrile in water with 0.1% trifluoroacetic acid) gave the title product. ¹H NMR (400 MHz, DMSO-d₆) δ 11.66 (s, 1H), 8.30 (d, J=5.0 Hz, 1H), 8.24 (d, J=5.7 Hz, 1H), 7.89 (s, 3H), 7.68-7.55 (m, 4H), 7.51 (d, J=8.5 Hz, 3H), 7.38 (d, J=8.3 Hz, 2H), 7.12 (d, J=4.9 Hz, 1H), 6.21 (s, 1H), 4.16 (dd, J=13.6, 6.0 Hz, 1H), 3.94 (dd, J=13.6, 4.9 Hz, 1H), 3.53-3.35 (m, 2H). LCMS (m/z [M+H]⁺). 461.2

### Synthesis of Compound 1030 and Intermediates Thereof

Compound 1030 can be prepared as illustrated in Scheme 14 below.

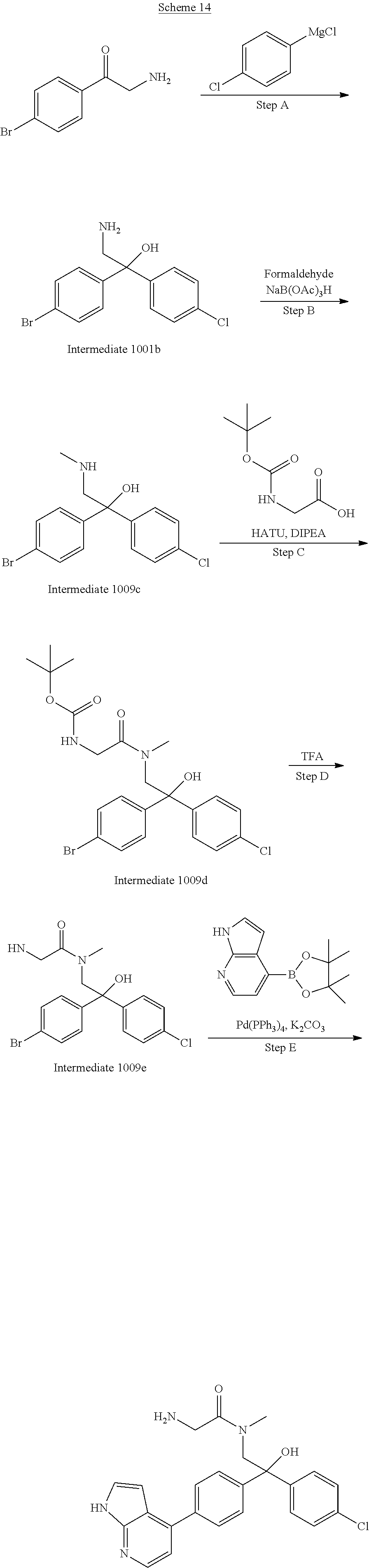

### Intermediate 1009c (Scheme 14): 1-(4-bromophenyl)-1-(4-chlorophenyl)-2-(methylamino)ethan-1-ol

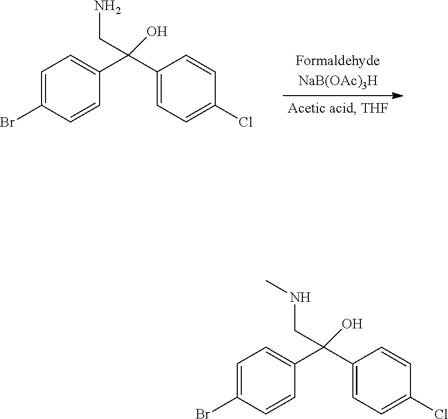 [SMILES: NCC(O)(c1ccc(Cl)cc1)c1ccc(Br)cc1 ; CNCC(O)(c1ccc(Cl)cc1)c1ccc(Br)cc1]

Step B: To a solution of 2-amino-1-(4-bromophenyl)-1-(4-chlorophenyl)ethan-1-ol (Intermediate 1001b, See Scheme 6, Step A), (0.357 grams, 1.07 mmol) in tetrahydrofuran (5.0 ml) at room temperature was added formaldehyde (0.020 g, 0.246 mmol, 37% by weight in water), followed by acetic acid (0.064 g, 1.072 mmol) and sodium triacetoxyborohydride (0.227 grams, 1.072 mmol). The reaction was stirred for 2 hours. It was then poured into saturated aqueous sodium bicarbonate and extracted into ethyl acetate. The organic phase was separated and the solvent removed by rotary evaporation to give the crude product, mixed with the unreacted starting material and the double-methylated product, which was immediately used in the next step. LCMS (m/z [M+H]⁺): 341.7

### Intermediate 1009d (Scheme 14): tert-butyl (2-((2-(4-bromophenyl)-2-(4-chlorophenyl)-2-hydroxyethyl)(methyl)amino)-2-oxoethyl)carbamate

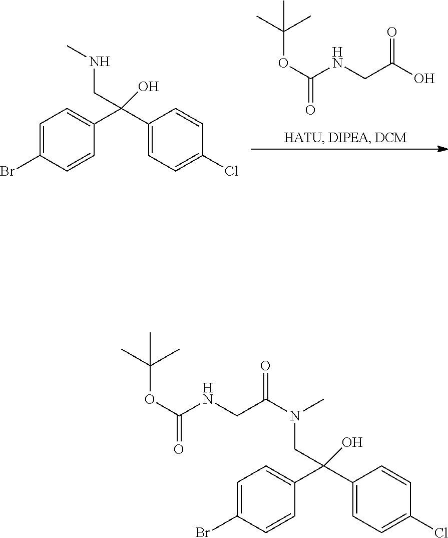 [SMILES: CNCC(O)(c1ccc(Cl)cc1)c1ccc(Br)cc1 ; CC(C)(C)OC(=O)NCC(=O)O ; CN(CC(O)(c1ccc(Cl)cc1)c1ccc(Br)cc1)C(=O)CNC(=O)OC(C)(C)C]

Step C: To a solution of: 1-(4-bromophenyl)-1-(4-chlorophenyl)-2-(methylamino)ethan-1-ol (0.365 grams, 1.07 mmol) in dichloromethane (20.0 ml) was added (tert-butoxycarbonyl)glycine (0.188 grams, 1.07 mmol), followed by 1-[bis(dimethylamino)methylene]-1H-1,2,3-triazolo[4,5-b]pyridinium 3-oxide hexafluorophosphate (0.407 g, 1.07 mmol), then diisopropylethylamine (0.222 grams, 1.718 mmol, 0.300 ml). The reaction was stirred for 1 hour at room temperature. The reaction was then poured into an aqueous saturated sodium bicarbonate solution, and extracted into ethyl acetate. The organic phase was separated, and the solvent removed by rotary evaporation. Purification by silica gel chromatography (0% to 100% ethyl acetate in hexanes) gave the title product. ¹H NMR (400 MHz, Chloroform-d) δ 7.49-7.41 (m, 2H), 7.40-7.33 (m, 2H), 7.33-7.27 (m, 4H), 5.39 (s, 1H), 4.20 (s, 2H), 3.89 (d, J=3.8 Hz, 2H), 2.50 (s, 3H), 1.45 (s, 9H). LCMS (m/z [M+Na]⁺): 520.7

### Intermediate 1009d (Scheme 14): 2-amino-N-(2-(4-bromophenyl)-2-(4-chlorophenyl)-2-hydroxyethyl)-N-methylacetamide

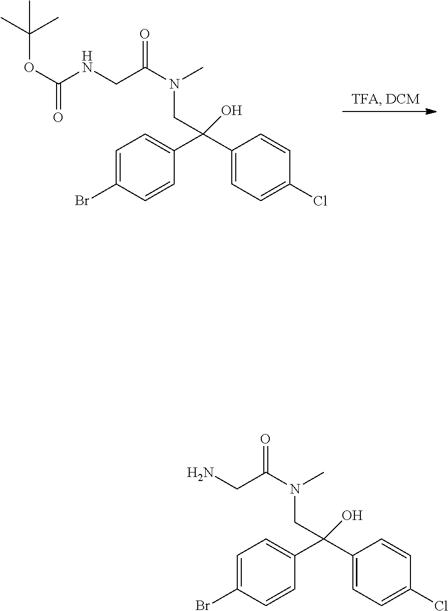 [SMILES: CN(CC(O)(c1ccc(Cl)cc1)c1ccc(Br)cc1)C(=O)CNC(=O)OC(C)(C)C ; CN(CC(O)(c1ccc(Cl)cc1)c1ccc(Br)cc1)C(=O)CN]

Step D: tert-butyl (2-((2-(4-bromophenyl)-2-(4-chlorophenyl)-2-hydroxyethyl)(methyl)amino)-2-oxoethyl)carbamate (0.075 grams, 0.151 mmol) was placed in a vial. Dichloromethane (1.0 ml) and trifluoroacetic acid (1.0 ml) were added to the vial and the reaction was stirred at room temperature for 30 minutes. The solvent and trifluoroacetic acid were then removed by rotary evaporation to give the crude product. LCMS (m/z [M+H]⁺): 398.7

### Compound 1030

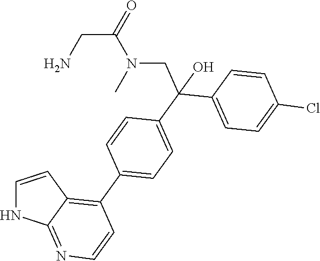 [SMILES: CN(CC(O)(c1ccc(Cl)cc1)c1ccc(-c2ccnc3[nH]ccc23)cc1)C(=O)CN]

### N-(2-(4-(1H-pyrrolo[2,3-b]pyridin-4-yl)phenyl)-2-(4-chlorophenyl)-2-hydroxyethyl)-2-amino-N-methylacetamide

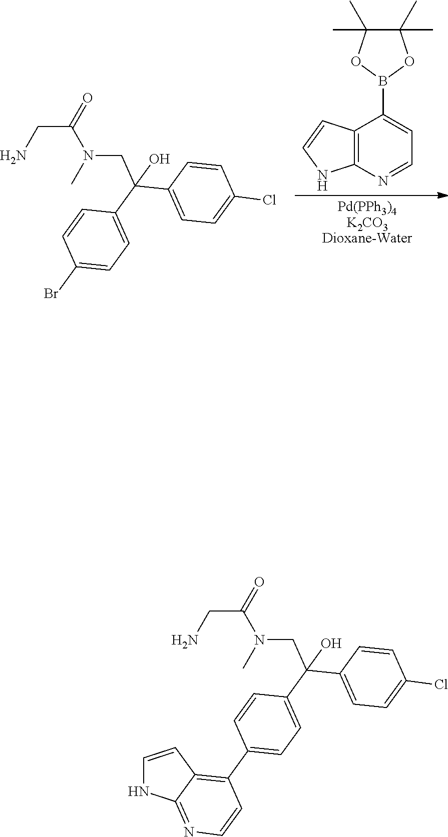

Step E: To a 2 mL microwave vial containing a stirbar was added 2-amino-N-(2-(4-bromophenyl)-2-(4-chlorophenyl)-2-hydroxyethyl)-N-methylacetamide (0.060 grams, 0.151 mmol), followed by 4-(4,4,5,5-tetramethyl-1,3,2-dioxaborolan-2-yl)-1H-pyrrolo[2,3-b]pyridine (0.037 grams, 0.151 mmol), potassium carbonate (0.100 grams, 0.724 mmol) and tetrakis(triphenylphosphine)-palladium(0) (0.020 grams, 0.017 mmol). The vial was sealed, and 1,4-dioxane (1.5 ml) followed by water (0.5 ml) was added. The reaction mixture had nitrogen gas bubbled through it for 5 minutes at room temperature. It was then heated by microwave irradiation to 150° C. for 30 minutes. The vial was cooled, and the solvent was removed by rotary evaporation. The reaction mixture was then dissolved in methanol (˜2 ml) and filtered. Purification by reverse phase chromatography (5% to 100% acetonitrile in water with 0.1% trifluoroacetic acid) gave the title product. ¹H NMR (400 MHz, DMSO-d₆) δ 11.83 (s, 1H), 8.28 (t, J=4.5 Hz, 1H), 7.94 (s, 3H), 7.77 (dd, J=12.6, 8.1 Hz, 2H), 7.63 (d, J=8.1 Hz, 2H), 7.54 (dt, J=7.9, 3.7 Hz, 3H), 7.42 (dd, J=29.2, 8.4 Hz, 2H), 7.20 (dd, J=7.7, 5.0 Hz, 1H), 6.61 (s, 1H), 6.42 (s, 1H), 4.21 (d, J=16.1 Hz, 1H), 3.98-3.61 (m, 2H), 2.87 (s, 1H). LCMS (m/z [M+H]⁺): 435.2

### Example 8: Dual LATS/AKT Inhibitors: Biological Evaluation

### In Vitro Kinase Inhibition Assays

Biochemical kinase inhibitory data was obtained for various exemplary compounds prepared according to the above. Compounds were evaluated using the RBC HotSpot Kinase Assay Protocol (Anastassiadis T, et al. Comprehensive assay of kinase catalytic activity reveals features of kinase inhibitor selectivity. Nat Biotechnol. 2011 Oct. 30; 29(11):1039-45), as described below.

The reagents used are as follows: Base Reaction buffer; 20 mM Hepes (pH 7.5), 10 mM MgCl2, 1 mM EGTA, 0.01% Brij35, 0.02 mg/ml BSA, 0.1 mM Na3VO4, 2 mM DTT, 1% DMSO. Required cofactors were added individually to each kinase reaction.

The reaction procedure was as follows:
1) Substrates were prepared in freshly prepared Reaction Buffer.
2) Any required cofactors were delivered to the substrate solution above.
3) Kinase was delivered into the substrate solution and gently mixed.
4) Compounds were delivered in 100% DMSO into the kinase reaction mixture by Acoustic technology (Echo550; nanoliter range), followed by incubation for 20 min at room temp.
5) ³³P-ATP was delivered into the reaction mixture to initiate the reaction.
6) The mixture was incubated for 2 hours at room temperature.
7) Kinase activity was detected by P81 filter-binding method.

Using the above functional assay, the inhibitory activity of the compounds for LATS1/2 versus AKT was characterized. The assays were typically run in a 10-point curve, in singlet, to produce a titration curve from which an IC₅₀ value was obtained. The IC₅₀ values are reported in Table 32.

### Ex Vivo Kinase Inhibition Assays

Kinase cellular potency data were obtained for various exemplary compounds, using the Reaction Biology NanoBRET™ assay protocol described below. The NanoBRET™ assay measures kinase engagement in real time in the context of the intact cell. The assay uses a Kinase-NanoLuc® fusion vector expressing a kinase protein to which a luciferase tag has been added, a cell-permeant fluorescent NanoBRET™ tracer, a NanoLuc® substrate, and an extracellular NanoLuc® inhibitor. Upon expression of the luciferase-tagged kinase, cells will produce a strong BRET signal only in the presence of the NanoBRET™ tracer. The extracellular NanoLuc® inhibitor ensures that the BRET signal observed emanates only from live cells. Because the BRET signal has tight distance constraints, addition of the test compound will decrease the BRET signal if the compound competes with the NanoBRET™ tracer for binding to the kinase domain. Under the appropriate tracer conditions established by the manufacturer, quantitative intracellular affinity and relative potency can then be determined using Mass Action model equations.

HEK-293 cells were purchased from ATCC. FuGENEHD Transfection Reagent, Kinase-NanoLuc® fusion plasmids, Transfection Carrier DNA, NanoBRET™ Tracers and dilution buffer, NanoBRET™ Nano-Glo Substrate, Extracellular NanoLuc® Inhibitor were obtained from Promega.

Assays were conducted following Promega assay protocol with some modifications as described further below. HEK-293 Cells were transiently transfected with Kinase-NanoLuc® Fusion Vector DNA by FuGENE HD Transfection Reagent. Test compounds were delivered into 384 well assay plate by Echo 550 (Labcyte Inc, Sunnyvale, CA). Transfected cells were harvested and mixed with NanoBRET™ Tracer Reagent and dispensed into 384 well plates and incubated at 37° C. in 5% CO₂ cell culture incubator for 1 hour. The NanoBRET™ Nano-Glo Substrate plus Extracellular NanoLuc® Inhibitor Solution were added into the wells of the assay plate and incubated for 2-3 minutes at room temperature. The donor emission wavelength (460 nm) and acceptor emission wavelength (600 nm) were measured in the EnVision plate reader. The BRET Ratios were calculated. BRET Ratio=[(Acceptor sample÷Donor sample)−(Acceptor no-tracer control÷Donor no-tracer control)]. The IC₅₀ values of compounds were calculated with Prism GraphPad program.

### NanoBRET™ Target Engagement Assay Protocol:

1. Transient Transfection of HEK-293 Cells NanoLuc® Fusion Vector DNA
   1). Cultivate HEK-293 cells (70-80% confluence) appropriately prior to assay. Trypsinize and collect HEK-293 cells.
   2). Prepare lipid: DNA complexes as follows:
      a. Prepare a 10 g/ml solution of DNA in Opti-MEM™ without serum that consists of the following ratios of carrier DNA and DNA encoding NanoLuc® fusion. 9.0 g/mL of Transfection Carrier DNA, 1.0 g/mL of NanoLuc® fusion vector DNA and 1 mL of Opti-MEM™ without phenol red. Mix thoroughly.
      b. Add 30 μl of FuGENE HD Transfection Reagent into each milliliter of DNA mixture to form lipid: DNA complex.
      c. Mix by inversion 10 times.
      d. Incubate at ambient temperature for 20 minutes to allow complexes to form.
   3). In a sterile, conical tube, mix 1 part of lipid: DNA complex with 20 parts of HEK-293 cells in suspension. Mix gently by inversion 5 times.
   4). Dispense cells+lipid: DNA complex into a sterile tissue culture dish and incubate for 22-24 hours.
2. Addition of Test Compounds (dry plate shooting): Each test compound is delivered from the compound source plate to the wells of 384-well white NBS plate by Echo 550.
3. Preparation of Cells with NanoBRET™ Tracer Reagent
   1). Remove medium from dish with transfected HEK-293 cells via aspiration, trypsinize and allow cells to dissociate from the dish.
   2). Neutralize trypsin using medium containing serum and centrifuge at 200× g for 5 minutes to pellet the cells. Adjust the cell density to 2×105 cells/mL in Opti-MEM™ without phenol red.
   3). Prepare Complete 20× NanoBRET™ Tracer Reagent with Tracer Dilution Buffer.
   4). Dispense one part of Complete 20× NanoBRET™ Tracer Reagent to 20 parts of cells in the tube. Mix gently by inversion 10 times.
   5). Dispense cell suspension into white, 384-well NBS plates. Incubate the plate at 37° C., 5% CO₂ for 1 hour.
Note: Prepare a separate set of samples without tracer for background correction steps.
4. NanoBRET™ Assay
   1). Remove plate from incubator and equilibrate to room temperature for 15 minutes.
   2). Prepare 3× Complete Substrate plus Inhibitor Solution in Assay Medium (Opti-MEM™ I Reduced Serum Medium, no phenol red) just before measuring BRET.
   3). Add 3× Complete Substrate plus Inhibitor Solution to each well of the 384-well plate. Incubate for 2-3 minutes at room temperature.
   4). Measure donor emission wavelength (460 nm) and acceptor emission wavelength (600 nm) using the Envision 2104 plate reader.
5. Determination of BRET Ratio: To generate raw BRET ratio values, divide the acceptor emission value (600 nm) by the donor emission value (460 nm) for each sample. To correct for background, subtract the BRET ratio in the absence of tracer (average of no-tracer control samples) from the BRET ratio of each sample.

$${NanoBRET}^{TM} ratio equation : BRET Ratio = ( Acceptor sample \div Donor sample ) \\ {NanoBRET}^{TM} ratio equation , including optional background correction : BRET Ratio = [ \text{} ( Acceptor sample \div Donor sample ) - ( Acceptor no ‐ tracer control \div Donor no ‐ tracer control ) ] \\ Normalized Bret Response equation ( \% ) : ( BRET Ratio of Compound Treated Sample / BRET Ratio of DMSO Control Sample ) * 100 \%$$

6. Determination of IC₅₀ Values: IC₅₀ curves are plotted and IC₅₀ values are calculated using the GraphPad Prism 4 program based on a sigmoidal dose-response equation.

IC₅₀ values as determined by the assays described above are provided in Table 32. All biological activities are IC₅₀, in nM.

TABLE 32

| | | | | | LATS1 | AKT |
|---|---|---|---|---|---|---|
| Compound | Structure | LATS1 | LATS2 | AKT | (Cellular) | (Cellular) |
| 1001 | 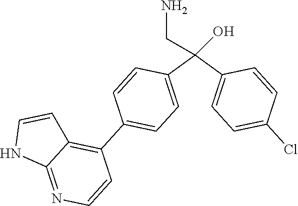 [SMILES: NCC(O)(c1ccc(Cl)cc1)c1ccc(-c2ccnc3[nH]ccc23)cc1] | 1.8 | 2.1 | 0.6 | 123 | 42 |
| 1002 | 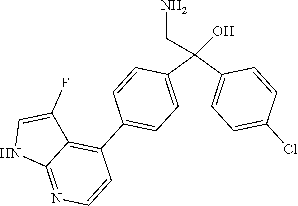 [SMILES: NCC(O)(c1ccc(Cl)cc1)c1ccc(-c2ccnc3[nH]cc(F)c23)cc1] | 5.7 | 11.1 | 4.6 | 55 | 27 |
| 1003 | 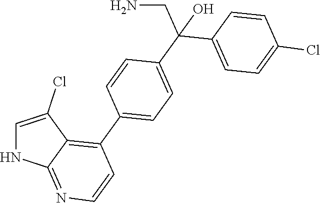 [SMILES: NCC(O)(c1ccc(Cl)cc1)c1ccc(-c2ccnc3[nH]cc(Cl)c23)cc1] | 31.7 | 12 | 3.2 | | |
| 1004 | 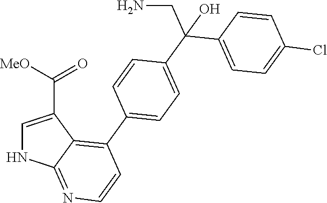 [SMILES: COC(=O)c1c[nH]c2nccc(-c3ccc(C(O)(CN)c4ccc(Cl)cc4)cc3)c12] | 4.0 | 5.3 | 18.1 | 2000 | 548 |
| 1005 | 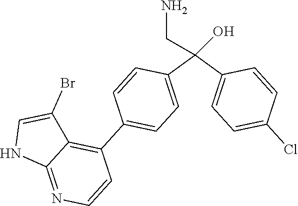 [SMILES: NCC(O)(c1ccc(Cl)cc1)c1ccc(-c2ccnc3[nH]cc(Br)c23)cc1] | 5.1 | 8.9 | 1.7 | 8.0 | 2.9 |
| 1006 | 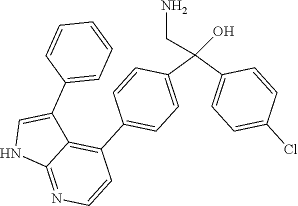 [SMILES: NCC(O)(c1ccc(Cl)cc1)c1ccc(-c2ccnc3[nH]cc(-c4ccccc4)c23)cc1] | 23 | 20 | 230 | 283 | 135 |
| 1007 | 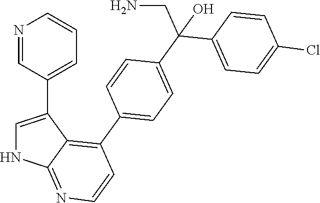 [SMILES: NCC(O)(c1ccc(Cl)cc1)c1ccc(-c2ccnc3[nH]cc(-c4cccnc4)c23)cc1] | 4.3 | 6.1 | 35.2 | 309 | 153 |
| 1009 | 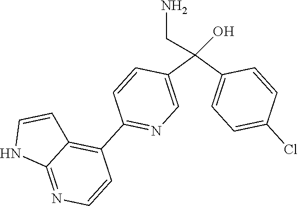 [SMILES: NCC(O)(c1ccc(Cl)cc1)c1ccc(-c2ccnc3[nH]ccc23)nc1] | <0.5 | <0.5 | 0.55 | 392 | 654 |
| 1009 | 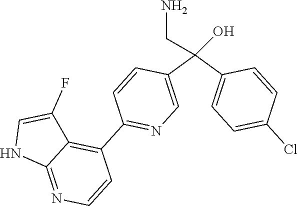 [SMILES: NCC(O)(c1ccc(Cl)cc1)c1ccc(-c2ccnc3[nH]cc(F)c23)nc1] | 3.7 | 1.4 | 7.6 | 2190 | 3520 |
| 1010 | 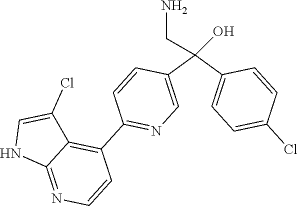 [SMILES: NCC(O)(c1ccc(Cl)cc1)c1ccc(-c2ccnc3[nH]cc(Cl)c23)nc1] | 0.5 | <0.5 | <0.5 | | |
| 1011 | 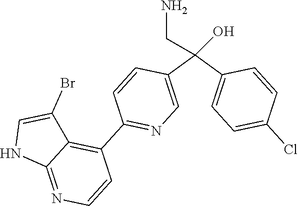 [SMILES: NCC(O)(c1ccc(Cl)cc1)c1ccc(-c2ccnc3[nH]cc(Br)c23)nc1] | 0.9 | <0.5 | 1.0 | 24 | 76.3 |
| 1012 | 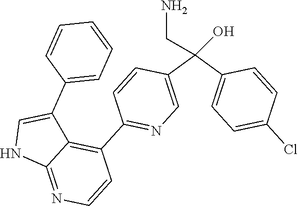 [SMILES: NCC(O)(c1ccc(Cl)cc1)c1ccc(-c2ccnc3[nH]cc(-c4ccccc4)c23)nc1] | 8.5 | 8.4 | 126 | | |
| 1013 | 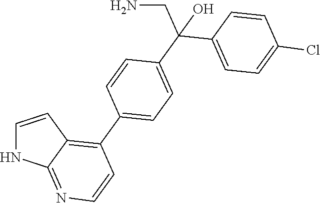 [SMILES: NCC(O)(c1ccc(Cl)cc1)c1ccc(-c2ccnc3[nH]ccc23)cc1] | 6.5 | 8.6 | 8.0 | | |
| 1014 | 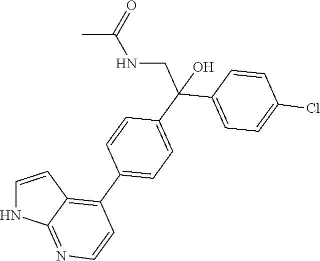 [SMILES: CC(=O)NCC(O)(c1ccc(Cl)cc1)c1ccc(-c2ccnc3[nH]ccc23)cc1] | 3.7 | 5.1 | 17.2 | | |
| 1015 | 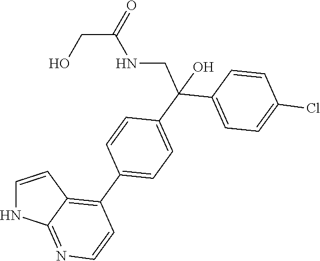 [SMILES: O=C(CO)NCC(O)(c1ccc(Cl)cc1)c1ccc(-c2ccnc3[nH]ccc23)cc1] | 4.0 | 3.7 | 5.9 | | |
| 1016 | 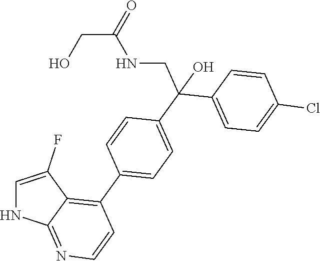 [SMILES: O=C(CO)NCC(O)(c1ccc(Cl)cc1)c1ccc(-c2ccnc3[nH]cc(F)c23)cc1] | 7.0 | 4.0 | 7.4 | | |
| 1017 | 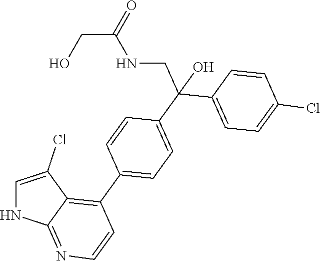 [SMILES: O=C(CO)NCC(O)(c1ccc(Cl)cc1)c1ccc(-c2ccnc3[nH]cc(Cl)c23)cc1] | 5.4 | 2.8 | 2.8 | | |
| 1018 | 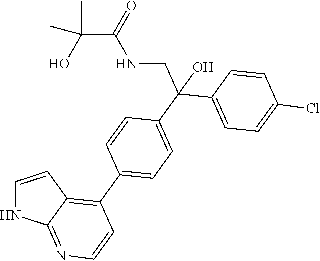 [SMILES: CC(C)(O)C(=O)NCC(O)(c1ccc(Cl)cc1)c1ccc(-c2ccnc3[nH]ccc23)cc1] | 18.7 | 10.1 | 73.6 | | |
| 1019 | 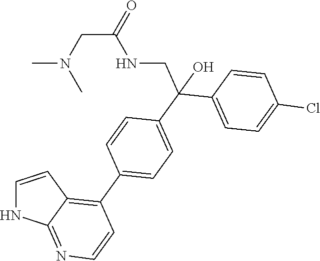 [SMILES: CN(C)CC(=O)NCC(O)(c1ccc(Cl)cc1)c1ccc(-c2ccnc3[nH]ccc23)cc1] | 4.0 | 5.6 | 5.4 | | |
| 1020 | 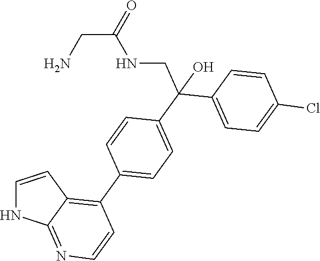 [SMILES: NCC(=O)NCC(O)(c1ccc(Cl)cc1)c1ccc(-c2ccnc3[nH]ccc23)cc1] | 3.0 | 2.4 | 1.4 | | |
| 1021 | 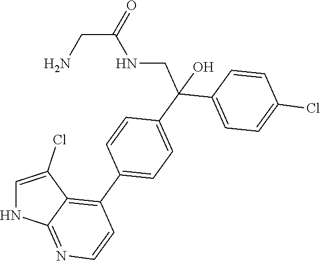 [SMILES: NCC(=O)NCC(O)(c1ccc(Cl)cc1)c1ccc(-c2ccnc3[nH]cc(Cl)c23)cc1] | 3.7 | 1.6 | <0.5 | 159 | 12 |
| 1022 | 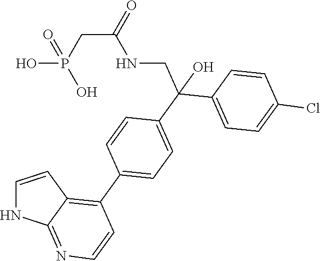 [SMILES: O=C(CP(=O)(O)O)NCC(O)(c1ccc(Cl)cc1)c1ccc(-c2ccnc3[nH]ccc23)cc1] | 9.6 | 12 | 3.1 | | |
| 1023 | 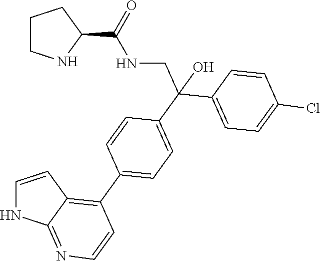 [SMILES: O=C(NCC(O)(c1ccc(Cl)cc1)c1ccc(-c2ccnc3[nH]ccc23)cc1)[C@@H]1CCCN1] | 3.4 | 4.6 | 9.2 | | |
| 1024 | 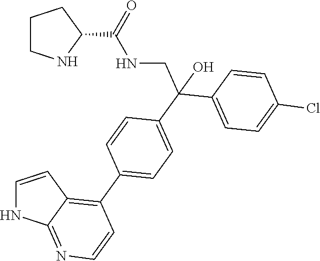 [SMILES: O=C(NCC(O)(c1ccc(Cl)cc1)c1ccc(-c2ccnc3[nH]ccc23)cc1)[C@H]1CCCN1] | 3.7 | 2.4 | 5.6 | | |
| 1025 | 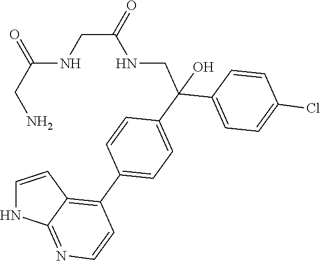 [SMILES: NCC(=O)NCC(=O)NCC(O)(c1ccc(Cl)cc1)c1ccc(-c2ccnc3[nH]ccc23)cc1] | 4.1 | 4.5 | 3.1 | | |
| 1026 | 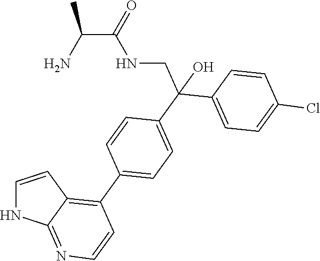 [SMILES: C[C@H](N)C(=O)NCC(O)(c1ccc(Cl)cc1)c1ccc(-c2ccnc3[nH]ccc23)cc1] | 3.8 | 2.3 | 1.2 | | |
| 1027 | 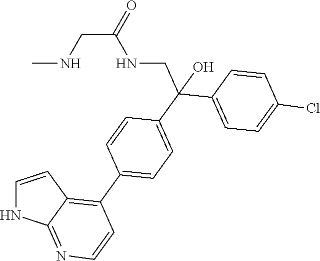 [SMILES: CNCC(=O)NCC(O)(c1ccc(Cl)cc1)c1ccc(-c2ccnc3[nH]ccc23)cc1] | 4.1 | 3.0 | 1.3 | | |
| 1028 | 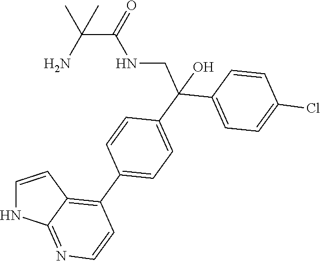 [SMILES: CC(C)(N)C(=O)NCC(O)(c1ccc(Cl)cc1)c1ccc(-c2ccnc3[nH]ccc23)cc1] | 1.9 | 2.4 | 1.9 | | |
| 1029 | 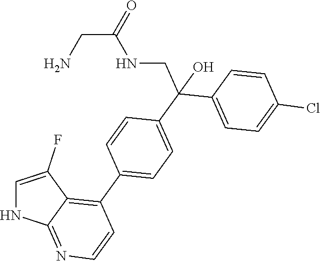 [SMILES: NCC(=O)NCC(O)(c1ccc(Cl)cc1)c1ccc(-c2ccnc3[nH]cc(F)c23)cc1] | 2.6 | 1.3 | 0.7 | | |
| 1030 | 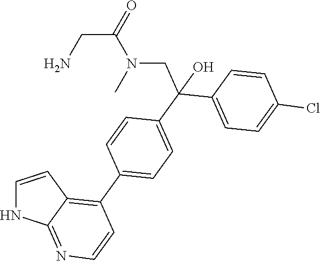 [SMILES: CN(CC(O)(c1ccc(Cl)cc1)c1ccc(-c2ccnc3[nH]ccc23)cc1)C(=O)CN] | 4.4 | 5.1 | 1.1 | | |

### Example 9: Nitrile LATS Inhibitors: General Methods

All commercially available reagents and solvents were purchased and used without further purification. All microwave reactions were carried out in a sealed microwave vial equipped with a magnetic stir bar and heated in a Biotage Initiator Microwave Synthesizer. 1H NMR spectra were recorded on Varian 400 MHz spectrometers in CD3OD, CD3CN, CDCl3, or D6-DMSO as indicated. For spectra recorded in CD3OD, chemical shifts are reported in ppm with CD₃OD (3.31 ppm) as reference for 1H NMR spectra. For spectra recorded in CDCl3, chemical shifts are reported in ppm relative to deuterochloroform (7.26 ppm for 1H NMR). For spectra recorded in CD3CN, chemical shifts are reported in ppm relative to CD3CN (1.93 ppm for 1H NMR). For spectra in D6-DMSO chemical shifts are reported in ppm relative to D6-DMSO (2.50 ppm for 1H NMR). The coupling constants (J value) are reported as Hertz (Hz). The splitting patterns of the peaks were described as: singlet (s); doublet (d); triplet (t); quartet (q); multiplet (m) and septet (septet).

Compounds were analyzed on an Agilent 1200 series LC/MS equipped with a Luna C18 (3 mm×75 mm, 3 μm) reversed-phase column with UV detection at λ=220 nm and λ=254 nm. The mobile phase consisted of water containing 0.05% trifluoroacetic acid as component A and acetonitrile containing 0.025% trifluoroacetic acid as component B. A linear gradient was run as follows: 0 min 4% B; 7 min 100% B; 8 min 100% B at a flow rate of 0.8 ml/min.

Reverse phase chromatography for purification purposes was performed on a Waters semi-preparative HPLC equipment. The column used was a Phenomenex Luna C18 (5 μm, 30×75 mm) at a flow rate of 45 mL/min. The mobile phase consisted of acetonitrile and water (each containing 0.1% trifluoroacetic acid, 0.1% formic acid, or 0.1% ammonium hydroxide, as indicated). A gradient of 5%-100% acetonitrile in water was used during the purification. Fraction collection was triggered by UV detection (220 nm).

### Example 10: Nitrile LATS Inhibitors: Synthesis

Certain compounds of the invention were synthesized by the following synthetic protocols.

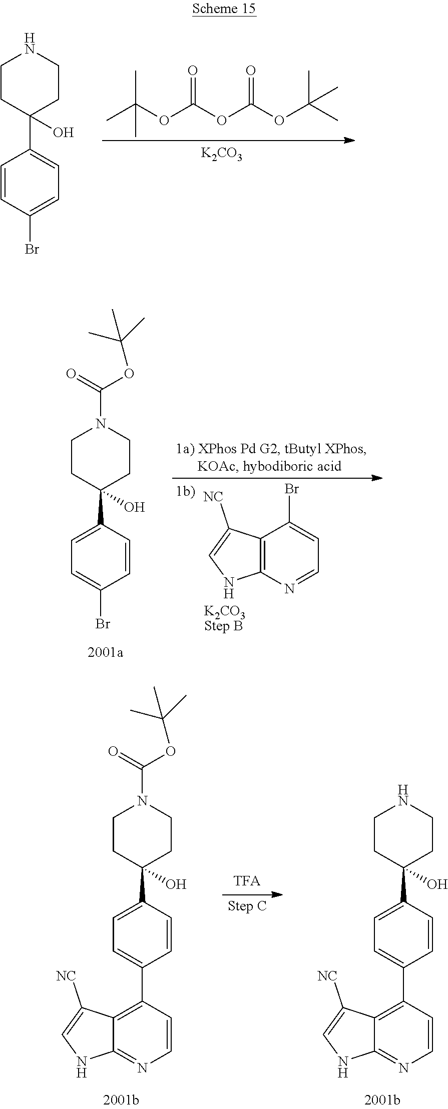

### Synthesis of Intermediates and Compounds of Scheme 15

### Intermediate 2001a (Scheme 15): tert-butyl 4-(4-bromophenyl)-4-hydroxypiperidine-1-carboxylate

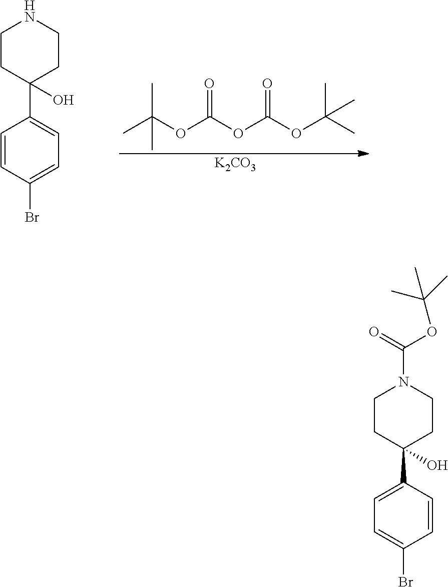

Step A: A round bottom flask containing a stirbar was charged with 4-(4-bromophenyl)piperidin-4-ol (0.500 g, 1.95 mmol), followed by potassium carbonate (0.270 g, 1.95 mmol) and Boc-anhydride (0.426 g, 1.95 mmol). Then tetrahydrofuran (15.0 ml) followed by water (5.0 ml) was added to the flask. The reaction was stirred at 23° C. for 16 hours. The reaction was then extracted into ethyl acetate, and washed with a saturated solution of sodium bicarbonate. The organic phase was taken, and the solvent removed by rotary evaporation. Purification by silica gel chromatography (0% to 100% ethyl acetate in hexanes) gave the title product. LC-MS (m/z [M−OC(CH₃)₃]⁺): 284.0

### Intermediate 2001b (Scheme 15): tert-butyl 4-(4-(3-cyano-1H-pyrrolo[2,3-b]pyridin-4-yl)phenyl)-4-hydroxypiperidine-1-carboxylate

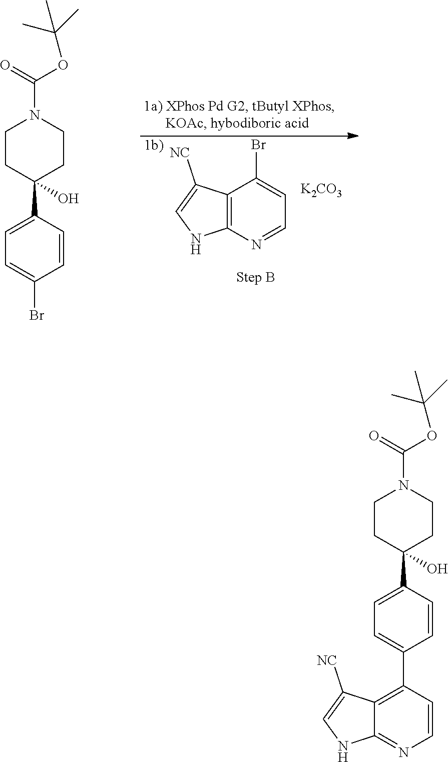

Step B: A 5 mL microwave vial with a stirbar was charged with tert-butyl 4-(4-bromophenyl)-4-hydroxypiperidine-1-carboxylate (0.107 g, 0.300 mmol), followed by hypodiboric acid (0.081 g, 0.900 mmol), potassium acetate (0.088 g, 0.900 mmol), chloro(2-dicyclohexylphosphino-2′,4′,6′-triisopropyl-1,1′-biphenyl)[2-(2′-amino-1,1′-biphenyl)]palladium(II) (0.012 g, 0.015 mmol, Xphos Pd G2 catalyst) and 2-di-tert-butylphosphino-2′,4′,6′-triisopropylbiphenyl (0.013 g, 0.030 mmol, tert-butyl Xphos). The vial was sealed, evacuated, and backfilled with argon. Than ethanol (3.0 ml, previously degassed via argon bubbling) was added via syringe. The reaction was then heated to 80° C. for 30 minutes by microwave irradiation. The vial was allowed to cool. Potassium carbonate (0.124 g, 0.900 mmol) in water (1.0 ml, degassed by argon bubbling) was added to the vial via syringe. Then 4-bromo-1H-pyrrolo[2,3-b]pyridine-3-carbonitrile (0.080 g, 0.360 mmol) dissolved in 2.0 mL of a 1:1 mixture of tetrahydrofuran and ethanol (both degassed by argon bubbling) was added to the vial via syringe. The reaction was then heated to 100° C. for a further 30 minutes via microwave irradiation. The vial was allowed to cool. The reaction was then extracted into ethyl acetate and washed with saturated sodium bicarbonate. The organic phase was taken, and the solvent removed by rotary evaporation. Purification by reverse phase column chromatography (5% to 100% acetonitrile in water, with 0.1% trifluoroacetic acid modifier) gave the desired product. ¹H NMR (400 MHz, Methanol-d₄) δ 8.26 (d, J=5.0 Hz, 1H), 8.14 (s, 1H), 7.62 (s, 4H), 7.08 (d, J=5.0 Hz, 1H), 4.00 (d, J=13.1 Hz, 2H), 2.02 (td, J=13.2, 4.7 Hz, 2H), 1.89 (s, 3H), 1.79 (d, J=13.4 Hz, 2H), 1.49 (s, 9H). LC-MS (m/z [M+H]⁺): 419.2

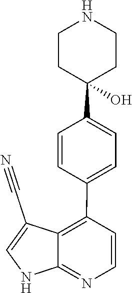

### Compound 2001

### 4-(4-(4-hydroxypiperidin-4-yl)phenyl)-1H-pyrrolo[2,3-b]pyridine-3-carbonitrile

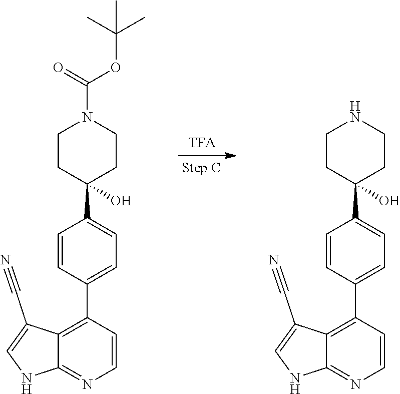

Step C: A vial containing a stirbar was charged with tert-butyl 4-(4-(3-cyano-1H-pyrrolo[2,3-b]pyridin-4-yl)phenyl)-4-hydroxypiperidine-1-carboxylate (0.040 g, 0.096 mmol) followed by dichloromethane (1.0 ml). Then trifluoroacetic acid (1.48 g, 13.0 mmol, 1.00 ml) was added via syringe. The vial was capped and the reaction was stirred for 0.5 hours at 23° C. The stirbar was then removed and the solvent and trifluoroacetic acid were removed by rotary evaporation. The crude product was purified by reverse phase high pressure liquid chromatography (5% to 100% acetonitrile in water with 0.1% trifluoroacetic acid modifier) to give the title product. ¹H NMR (400 MHz, DMSO-d₆) δ 13.00 (s, 1H), 8.53 (d, J=3.1 Hz, 1H), 8.45 (d, J=4.9 Hz, 1H), 7.72-7.57 (m, 4H), 7.26 (d, J=4.9 Hz, 1H), 5.60 (s, 1H), 3.31-3.19 (m, 4H), 2.17 (td, J=13.2, 5.4 Hz, 2H), 1.85 (d, J=14.0 Hz, 2H). LC-MS (m/z [M+H]⁺). 319.1

### Synthesis of Compounds of General Formula N2

Compounds of general formula N2 can be prepared as illustrated in Scheme 16 below.

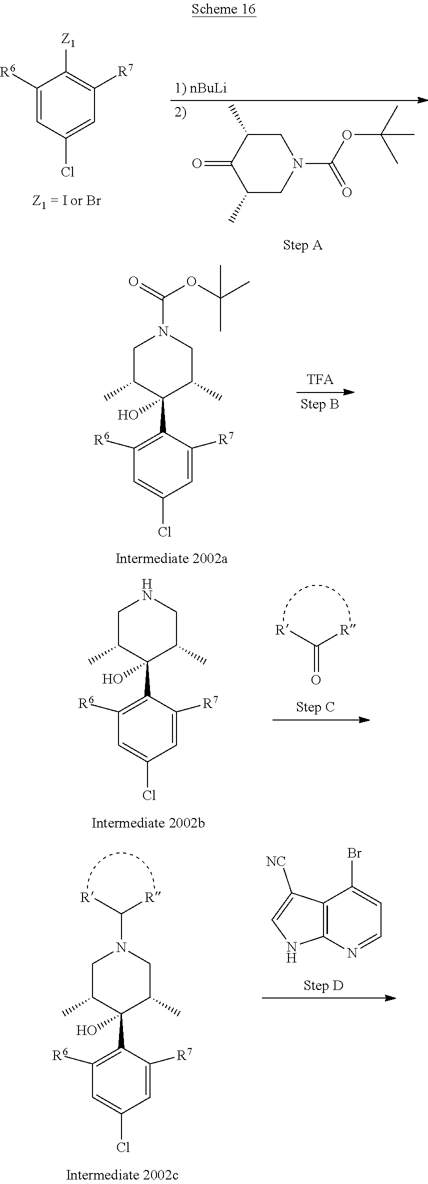

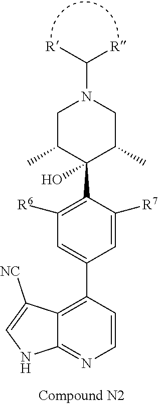

### Synthesis of Intermediates and Compounds of Scheme 16

### Intermediate 2002a-1 (Scheme 16): tert-butyl (3R,4s,5S)-4-(4-chloro-2-fluoro-6-methylphenyl)-4-hydroxy-3,5-dimethylpiperidine-1-carboxylate

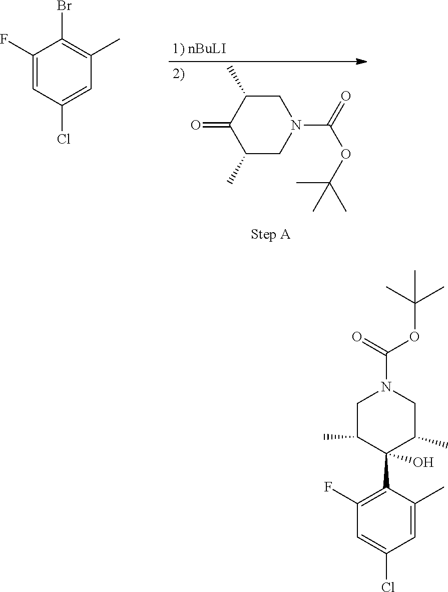

Step A: An oven-dried flask was charged with 2-bromo-5-chloro-1-fluoro-3-methylbenzene (0.450 g, 1.98 mmol) and tetrahydrofuran (4.0 mL), under a nitrogen atmosphere. The resulting solution was cooled down to −78° C. using an dry ice-acetone bath. Then n-butyl lithium (2.5 M in hexanes) (0.72 mL, 1.79 mmol) was added over 15 seconds. The reaction was allowed to proceed for 10 minutes. Then, a solution containing tert-butyl (3R,5S)-3,5-dimethyl-4-oxopiperidine-1-carboxylate (0.45 g, 1.98 mmol) in tetrahydrofuran (5.0 mL) was added slowly over approximately 30 seconds. The reaction mixture was stirred for 2 hours at −78° C. The reaction mixture was then quenched by being poured into a saturated aqeuous solution of sodium bicarbonate. The reaction mixture was extracted into ethyl acetate. The organic phase was taken and the solvent removed by rotary evaporation to give the crude product. Purification by silica gel chromatography (10% to 40% ethyl acetate in hexanes) provided the title compound. Relative stereochemistry was determined by NOE. ¹H NMR (400 MHz, Chloroform-d) δ 6.95-6.82 (m, 2H), 4.35 (bs, 1H), 3.84 (bs, 2H), 2.76 (bs, 2H), 2.59 (s, 3H), 2.57-2.43 (m, 2H), 1.47 (s, 9H), 0.70 (d, J=6.9 Hz, 6H). LC-MS (m/z [M−CO₂(CH₃)₃+2H]⁺): 272.1

### Intermediate 2002a-2 (Scheme 16)

Intermediate 2002a-2 was prepared using an analogous method to that of Intermediate 2002a-1 and is shown in Table 33.

TABLE 33

| Compound | Structure | IUPAC name | LCMS m/z |
|---|---|---|---|
| 2002a-2 | 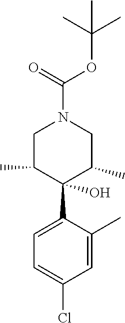 | tert-butyl (3R,4s,5S)- 4-(4-chloro-2-methyl- phenyl)-4-hydroxy- 3,5-dimethylpiperidine- 1-carboxylate | LC-MS (m/z [M −CO₂(CH₃)₃ + 2H]⁺): 254.1 |

### Intermediate 2002b-1 (Scheme 16): (3R,4s,5S)-4-(4-chloro-2-fluoro-6-methylphenyl)-3,5-dimethylpiperidin-4-ol

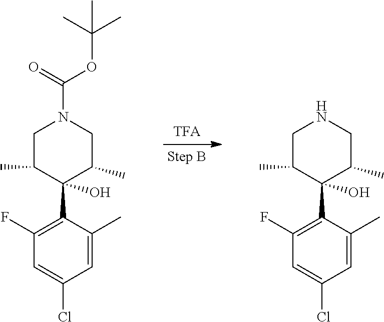

Step B: A vial containing a stirbar was charged with tert-butyl (3R,4s,5S)-4-(4-chloro-2-fluoro-6-methylphenyl)-4-hydroxy-3,5-dimethylpiperidine-1-carboxylate (0.750 g, 2.02 mmol) followed by dichloromethane (5.0 ml). Then trifluoroacetic acid (7.40 g, 64.9 mmol, 5.00 ml) was added via syringe. The vial was capped and the reaction was stirred for 0.5 hours at 23° C. The stirbar was then removed and the solvent and trifluoroacetic acid were removed by rotary evaporation. The crude product was treated with aqueous saturated sodium bicarbonate (50 mL), and extracted into ethyl acetate. The organic phase was taken and the solvent removed by rotary evaporation to give the product without further purification being necessary. Relative stereochemistry was confirmed by single crystal x-ray crystallography. ¹H NMR (400 MHz, Methanol-d₄) δ 6.99-6.93 (m, 2H), 2.89 (td, J=12.2, 3.0 Hz, 2H), 2.80 (dd, J=12.5, 4.4 Hz, 2H), 2.64 (s, 3H), 2.57 (dtd, J=11.4, 6.9, 4.5 Hz, 2H), 0.72 (d, J=6.9 Hz, 6H). ¹⁹F NMR (376 MHz, Methanol-d₄) δ −105.34 (d, J_{F-H}=12.9 Hz). LC-MS (m/z [M+H]⁺): 272.1

### Intermediate 2002b-2 (Scheme 16)

Intermediate 2002b-2 was prepared using an analogous method to that of Intermediate 2002b-1 and is shown in Table 34.

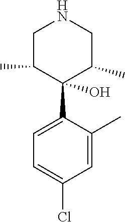

### Intermediate 2002c-1 (Scheme 16): 4-((3S,4s,5R)-4-(4-chloro-2-fluoro-6-methylphenyl)-4-hydroxy-3,5-dimethylpiperidin-1-yl)tetrahydro-2H-thiopyran 1,1-dioxide

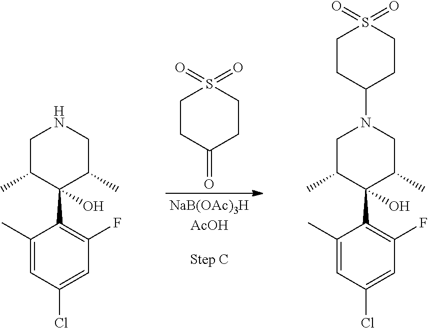

Step C: A 5 mL microwave vial with a stirbar was charged with (3R,4s,5S)-4-(4-chloro-2-fluoro-6-methylphenyl)-3,5-dimethylpiperidin-4-ol (0.124 g, 0.457 mmol), followed by tetrahydro-4H-thiopyran-4-one 1,1-dioxide (0.135 g, 0.914 mmol). Then tetrahydrofuran (4.0 ml) was added, followed by acetic acid (2.7 mg, 0.046 mmol, 2.6 uL). The reaction was set to stirring, and then sodium triacetoxyborohydride (0.291 g, 1.37 mmol) was added portionwise at room temperature. The vial was then sealed and heated in the microwave to 50° C. for 3 hours. The vial was allowed to cool, then the reaction was poured into saturated aqueous sodium bicarbonate. The mixture was extracted with ethyl acetate, the organic phase taken, and the solvent removed by rotary evaporation. Purification by silica gel chromatography (0% to 100% ethyl acetate in hexanes, then 0% to 10% methanol in dichloromethane) gave the title product. ¹H NMR (400 MHz, Chloroform-d) δ 6.90 (dd, J=10.8, 2.4 Hz, 2H), 3.42-3.35 (m, 1H), 3.22 (ddd, J=11.1, 7.3, 3.4 Hz, 2H), 3.01-2.90 (m, 3H), 2.68-2.61 (m, 1H), 2.58 (s, 3H), 2.57-2.52 (m, 2H), 2.35-2.23 (m, 5H), 2.23-2.12 (m, 1H), 0.71 (d, J=6.8 Hz, 6H). LC-MS (m/z [M+H]+): 404.1

### Intermediate 2002c-2 (Scheme 16)

Intermediate 2002c-2 was prepared using an analogous method to that of Intermediate 2002c-1 and is shown in Table 35.

TABLE 35

| Compound | Structure | IUPAC name | LCMS m/z |
|---|---|---|---|
| 2002c-2 | 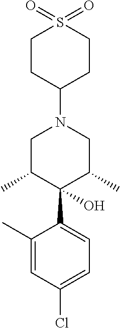 | 4-((3S,4s,5R)-4-(4- chloro-2-methylphen- yl)-4-hydroxy-3,5- dimethylpiperidin-1- yl)tetrahydro-2H- thiopyran 1,1-dioxide | LC-MS (m/z [[M + H]⁺): 386.1 |

### Compound 2002

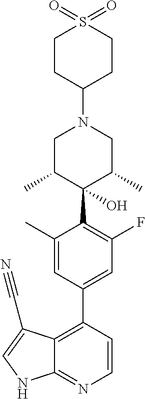 [SMILES: Cc1cc(-c2ccnc3[nH]cc(C#N)c23)cc(F)c1[C@@]1(O)[C@H](C)CN(C2CCS(=O)(=O)CC2)C[C@@H]1C]

### 4-(4-((3S,4s,5R)-1-(1,1-dioxidotetrahydro-2H-thiopyran-4-yl)-4-hydroxy-3,5-dimethylpiperidin-4-yl)-3-fluoro-5-methylphenyl)-1H-pyrrolo[2,3-b]pyridine-3-carbonitrile

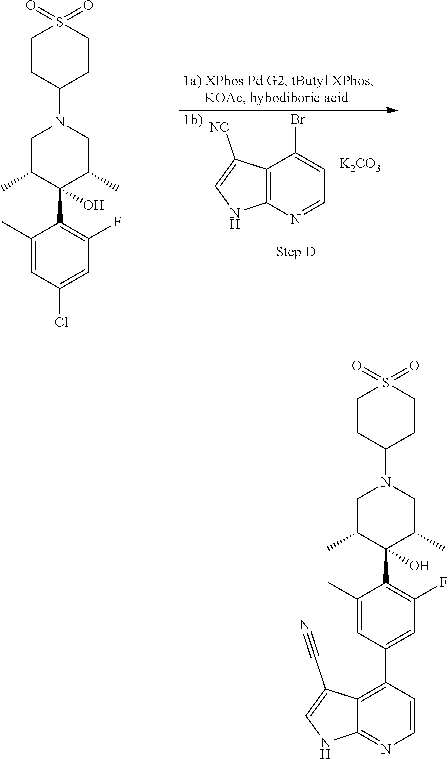

Step D: A 5 mL microwave vial with a stirbar was charged with 4-((3S,4s,5R)-4-(4-chloro-2-fluoro-6-methylphenyl)-4-hydroxy-3,5-dimethylpiperidin-1-yl)tetrahydro-2H-thiopyran 1,1-dioxide (0.075 g, 0.186 mmol) followed by hypodiboric acid (0.050 g, 0.557 mmol), potassium acetate (0.055 g, 0.557 mmol), chloro(2-dicyclohexylphosphino-2′,4′,6′-triisopropyl-1,1′-biphenyl)[2-(2′-amino-1,1′-biphenyl)]palladium(II) (0.007 g, 0.001 mmol, Xphos Pd G2 catalyst) and 2-di-tert-butylphosphino-2′,4′,6′-triisopropylbiphenyl (0.008 g, 0.002 mmol, tert-butyl Xphos). The vial was sealed, evacuated, and backfilled with argon. Then ethanol (3.0 ml, previously degassed via argon bubbling) was added via syringe. The reaction was then heated to 80° C. for 30 minutes by microwave irradiation. The vial was allowed to cool. Potassium carbonate (0.077 g, 0.557 mmol) in water (1.0 ml, degassed by argon bubbling) was added to the vial via syringe. Then 4-bromo-1H-pyrrolo[2,3-b]pyridine-3-carbonitrile (0.049 g, 0.22 mmol) dissolved in 2.0 mL of ethanol (degassed by argon bubbling) was added to the vial via syringe. The reaction was then heated to 100° C. for a further 30 minutes via microwave irradiation. The vial was allowed to cool. The reaction was then extracted into ethyl acetate and washed with saturated sodium bicarbonate. The organic phase was taken, and the solvent removed by rotary evaporation. Purification by reverse phase high pressure column chromatography (5% to 100% acetonitrile in water, with 0.1% trifluoroacetic acid modifier) gave the desired product. ¹H NMR (400 MHz, Methanol-d₄) δ 8.41 (d, J=4.9 Hz, 1H), 8.20 (s, 1H), 7.28 (d, J=5.0 Hz, 1H), 7.21 (d, J=2.0 Hz, 1H), 7.14 (dd, J=13.8, 2.0 Hz, 1H), 3.15 (d, J=4.7 Hz, 4H), 2.82-2.71 (m, 3H), 2.75 (s, 3H), 2.63-2.54 (m, 4H), 2.31-2.11 (m, 4H), 0.80 (d, J=6.8 Hz, 6H). LC-MS (m/z [M+H]⁺): 511.3

### Compound 2003

Compound 2003 was prepared using an analogous method to that of Compound 2002 and is shown in Table 36.

TABLE 36

| Compound | Structure | IUPAC name | ¹H NMR | LCMS m/z |
|---|---|---|---|---|
| 2003 | 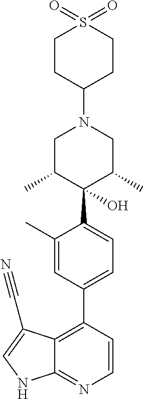 [SMILES: Cc1cc(-c2ccnc3[nH]cc(C#N)c23)ccc1[C@@]1(O)[C@H](C)CN(C2CCS(=O)(=O)CC2)C[C@@H]1C] | 4-(4-((3S,4s,5R)-1- (1,1-dioxidotetra- hydro-2H-thiopyran- 4-yl)-4-hydroxy-3,5- dimethylpiperidin- 4-yl)-3-methylphen- yl)-1H-pyrrolo[2,3- b]pyridine-3-carbo- nitrile | ¹H NMR (400 MHz, DMSO- d₆) δ 12.95 (s, 1H), 9.68 (s, 1H), 8.49 (d, J = 3.0 Hz, 1H), 8.44 (dd, J = 4.9, 1.3 Hz, 1H), 7.82 (d, J = 8.0 Hz, 1H), 7.49-7.35 (m, 2H), 7.30-7.21 (m, 1H), 5.25 (s, 1H), 3.65- 3.55 (m, 2H), 3.34-3.25 (m, 5H), 3.14 (q, J = 11.5 Hz, 2H), 2.86-2.76 (m, 2H), 2.68 (s, 1H), 2.55 (s, 2H), 2.47- 2.41 (m, 2H), 2.30-2.15 (m, 2H), 0.75 (d, J = 6.7 Hz, 1H), 0.63 (d, J = 6.8 Hz, 5H). | [M + H]⁺: 493.1 |

### Synthesis of Compounds of General Formula N3

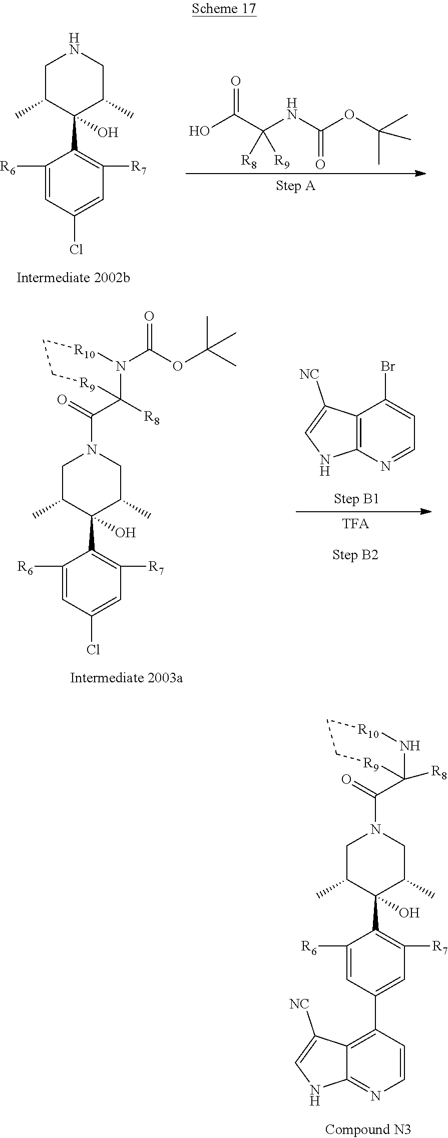

### Synthesis of Intermediates and Compounds of Scheme 17

### Intermediate 2003a-1 (Scheme 17): tert-butyl (2-((3S,4s,5R)-4-(4-chloro-2-fluoro-6-methylphenyl)-4-hydroxy-3,5-dimethylpiperidin-1-yl)-2-oxoethyl)carbamate

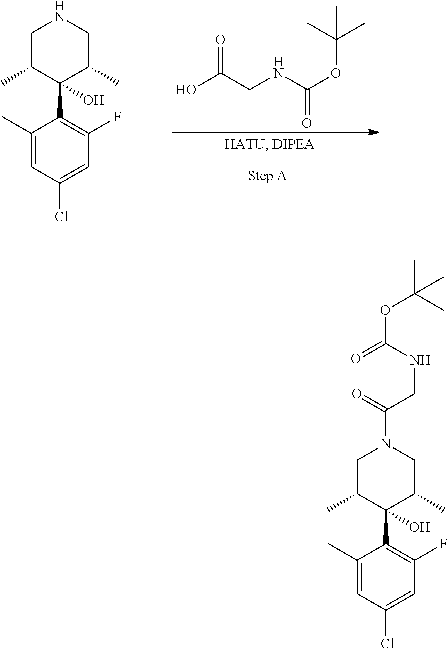

Step A: A vial containing a stirbar was charged with (3R,4s,5S)-4-(4-chloro-2-fluoro-6-methylphenyl)-3,5-dimethylpiperidin-4-ol (0.050 g, 0.183 mmol) followed by N-(tert-butoxycarbonyl)glycine (0.042 g, 0.238 mmol), then 1-[bis(dimethylamino)methylene]-1H-1,2,3-triazolo[4,5-b]pyridinium 3-oxid hexafluorophosphate (0.091 g, 0.238 mmol, HATU). Then tetrahydrofuran (5.0 ml) was added, followed by diisopropylethylamine (0.047 g, 0.366 mmol, 0.064 ml). The reaction was stirred at 23° C. for 3 h. The reaction was then poured into aqueous saturated sodium bicarbonate and extracted into ethyl acetate. The organic phase was taken and the solvent was removed by rotary evaporation. Purification by silica gel chromatography (0% to 100% ethyl acetate in hexanes) gave the title product. ¹H NMR (400 MHz, Chloroform-d) δ 6.90 (d, J=12.7 Hz, 2H), 5.58 (s, 1H), 4.38 (ddd, J=12.9, 4.9, 1.6 Hz, 1H), 4.00 (qd, J=16.7, 4.3 Hz, 2H), 3.46-3.26 (m, 1H), 3.14 (td, J=13.4, 12.6, 3.0 Hz, 1H), 2.77-2.65 (m, 1H), 2.60 (s, 3H), 2.57-2.46 (m, 2H), 1.46 (s, 9H), 0.75 (d, J=6.8 Hz, 6H). LC-MS (m/z [M+H]+): 429.2Xx

### Intermediates 2003a-2 to 2003a-17 (Scheme 17)

Intermediates 2003a-2 to 2003a-17 were prepared using an analogous method to that of Intermediate 2003a-1 and are shown in Table 37.

TABLE 37

| Compound | Structure | IUPAC name | LCMS m/z |
|---|---|---|---|
| 2003a-2 | 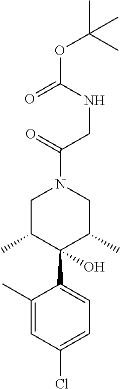 [SMILES: Cc1cc(Cl)ccc1[C@@]1(O)[C@H](C)CN(C(=O)CNC(=O)OC(C)(C)C)C[C@@H]1C] | tert-butyl (2-((3S,4s,5R)- 4-(4-chloro-2-methyl- phenyl)-4-hydroxy-3,5- dimethylpiperidin-1-yl)- 2-oxoethyl)carbamate | [M + H]⁺: 411.2 |
| 2003a-3 | 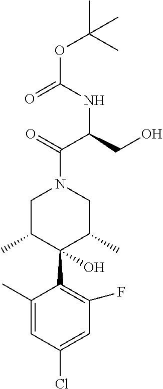 [SMILES: Cc1cc(Cl)cc(F)c1[C@@]1(O)[C@H](C)CN(C(=O)[C@H](CO)NC(=O)OC(C)(C)C)C[C@@H]1C] | tert-butyl ((S)-1- ((3S,4R,5R)-4-(4-chloro- 2-fluoro-6-methylphen- yl)-4-hydroxy-3,5- dimethylpiperidin-1-yl)- 3-hydroxy-1-oxopropan- 2-yl)carbamate | [M + H]⁺: 459.0 |
| 2003a-4 | 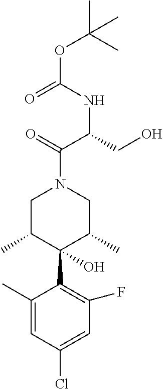 [SMILES: Cc1cc(Cl)cc(F)c1[C@@]1(O)[C@H](C)CN(C(=O)[C@@H](CO)NC(=O)OC(C)(C)C)C[C@@H]1C] | tert-butyl ((R)-1- ((3S,4S,5R)-4-(4-chloro- 2-fluoro-6-methylphen- yl)-4-hydroxy-3,5- dimethylpiperidin-1-yl)- 3-hydroxy-1-oxopropan- 2-yl)carbamate | [M + H]⁺: 459.0 |
| 2003a-5 | 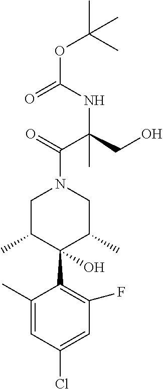 [SMILES: Cc1cc(Cl)cc(F)c1[C@@]1(O)[C@H](C)CN(C(=O)[C@](C)(CO)NC(=O)OC(C)(C)C)C[C@@H]1C] | tert-butyl (1-((3S,4s,5R)- 4-(4-chloro-2-fluoro-6- methylphenyl)-4- hydroxy-3,5-dimethyl- piperidin-1-yl)-3- hydroxy-2-methyl-1-oxo- propan-2-yl)carbamate | [M + H]⁺: 473.0 |
| 2003a-6 | 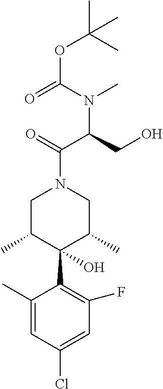 [SMILES: Cc1cc(Cl)cc(F)c1[C@@]1(O)[C@H](C)CN(C(=O)[C@H](CO)N(C)C(=O)OC(C)(C)C)C[C@@H]1C] | tert-butyl ((S)-1- ((3S,4R,5R)-4-(4-chloro- 2-fluoro-6-methylphen- yl)-4-hydroxy-3,5- dimethylpiperidin-1-yl)- 3-hydroxy-1-oxopropan- 2-yl)(methyl)carbamate | [M + H]⁺: 473.0 |
| 2003a-7 | 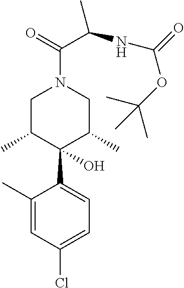 [SMILES: Cc1cc(Cl)ccc1[C@@]1(O)[C@H](C)CN(C(=O)[C@@H](C)NC(=O)OC(C)(C)C)C[C@@H]1C] | tert-butyl ((R)-1- ((3S,4S,5R)-4-(4-chloro- 2-methylphenyl)-4- hydroxy-3,5-dimethyl- piperidin-1-yl)-1-oxo- propan-2-yl)carbamate | [M + H]⁺: 425.0 |
| 2003a-8 | 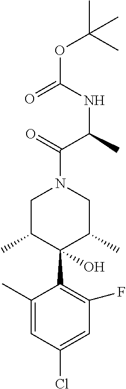 [SMILES: Cc1cc(Cl)cc(F)c1[C@@]1(O)[C@H](C)CN(C(=O)[C@H](C)NC(=O)OC(C)(C)C)C[C@@H]1C] | tert-butyl ((S)-1- ((3S,4R,5R)-4-(4-chloro- 2-fluoro-6-methylphen- yl)-4-hydroxy-3,5- dimethylpiperidin-1- yl)-1-oxopropan-2-yl)- carbamate | [M + H]⁺: 443.2 |
| 2003a-9 | 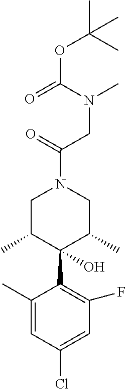 [SMILES: Cc1cc(Cl)cc(F)c1[C@@]1(O)[C@H](C)CN(C(=O)CN(C)C(=O)OC(C)(C)C)C[C@@H]1C] | tert-butyl (2-((3S,4s,5R)- 4-(4-chloro-2-fluoro- 6-methylphenyl)-4- hydroxy-3,5-dimethyl- piperidin-1-yl)-2-oxo- ethyl)(methyl)carbamate | [M + H]⁺: 443.2 |
| 2003a-10 | 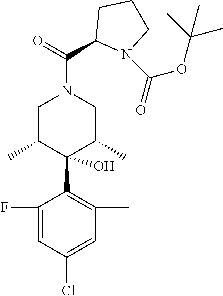 [SMILES: Cc1cc(Cl)cc(F)c1[C@@]1(O)[C@H](C)CN(C(=O)[C@H]2CCCN2C(=O)OC(C)(C)C)C[C@@H]1C] | tert-butyl (R)-2- ((3S,4S,5R)-4-(4-chloro- 2-fluoro-6-methylphen- yl)-4-hydroxy-3,5- dimethylpiperidine-1- carbonyl)pyrrolidine-1- carboxylate | [M + H]⁺: 469.0 |
| 2003a-11 | 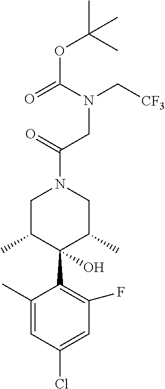 [SMILES: Cc1cc(Cl)cc(F)c1[C@@]1(O)[C@H](C)CN(C(=O)CN(CC(F)(F)F)C(=O)OC(C)(C)C)C[C@@H]1C] | tert-butyl (2-((3S,4s,5R)- 4-(4-chloro-2-fluoro- 6-methylphenyl)-4- hydroxy-3,5-dimethyl- piperidin-1-yl)-2-oxo- ethyl)(2,2,2-trifluoro- ethyl)carbamate | [M + H]⁺: 511.2 |
| 2003a-12 | 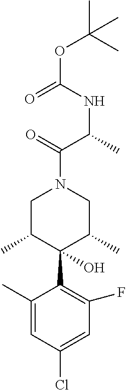 [SMILES: Cc1cc(Cl)cc(F)c1[C@@]1(O)[C@H](C)CN(C(=O)[C@@H](C)NC(=O)OC(C)(C)C)C[C@@H]1C] | tert-butyl ((R)-1- ((3S,4S,5R)-4-(4-chloro- 2-fluoro-6-methylphen- yl)-4-hydroxy-3,5- dimethylpiperidin-1- yl)-1-oxopropan-2-yl)- carbamate | [M + H]⁺: 443.2 |
| 2003a-13 | 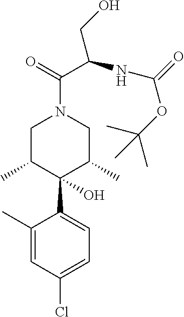 [SMILES: Cc1cc(Cl)ccc1[C@@]1(O)[C@H](C)CN(C(=O)[C@@H](CO)NC(=O)OC(C)(C)C)C[C@@H]1C] | tert-butyl ((R)-1- ((3S,4S,5R)-4-(4-chloro- 2-methylphenyl)-4- hydroxy-3,5-dimethyl- piperidin-1-yl)-3- hydroxy-1-oxopropan- 2-yl)carbamate | [M + H]⁺: 441.2 |
| 2003a-14 | 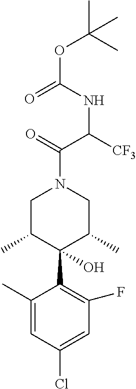 [SMILES: Cc1cc(Cl)cc(F)c1[C@@]1(O)[C@H](C)CN(C(=O)C(NC(=O)OC(C)(C)C)C(F)(F)F)C[C@@H]1C] | tert-butyl (3-((3S,4s,5R)- 4-(4-chloro-2-fluoro-6- methylphenyl)-4- hydroxy-3,5-dimethyl- piperidin-1-yl)-1,1,1- trifluoro-3-oxopropan- 2-yl)carbamate | [M − C(CH)₃ + H]⁺: 440.9 |
| 2003a-15 | 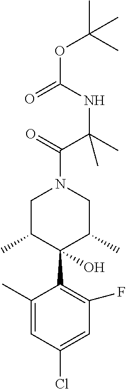 [SMILES: Cc1cc(Cl)cc(F)c1[C@@]1(O)[C@H](C)CN(C(=O)C(C)(C)NC(=O)OC(C)(C)C)C[C@@H]1C] | tert-butyl (1-((3S,4s,5R)- 4-(4-chloro-2-fluoro-6- methylphenyl)-4- hydroxy-3,5-dimethyl- piperidin-1-yl)-2-methyl- 1-oxopropan-2-yl)- carbamate | [M + H]⁺: 457.3 |
| 2003a-16 | 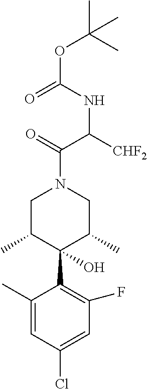 [SMILES: Cc1cc(Cl)cc(F)c1[C@@]1(O)[C@H](C)CN(C(=O)C(NC(=O)OC(C)(C)C)C(F)F)C[C@@H]1C] | tert-butyl (1-((3S,4s,5R)- 4-(4-chloro-2-fluoro-6- methylpheny1)-4- hydroxy-3,5-dimethyl- piperidin-1-yl)-3,3- difluoro-1-oxopropan-2- yl)carbamate | [M − C(CH)₃ + H]⁺: 422.9 |
| 2003a-17 | 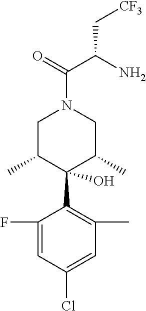 [SMILES: Cc1cc(Cl)cc(F)c1[C@@]1(O)[C@H](C)CN(C(=O)[C@@H](N)CC(F)(F)F)C[C@@H]1C] | (S)-2-amino-1- ((3S,4R,5R)-4-(4-chloro- 2-fluoro-6-methylphen- yl)-4-hydroxy-3,5- dimethylpiperidin-1- yl)-4,4,4-trifluorobutan- 1-one | [M −C(CH)₃ + H]⁺: 454.8 |

### Compound 2004

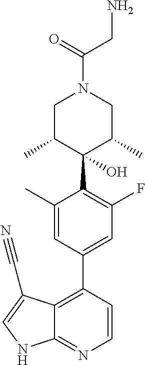 [SMILES: Cc1cc(-c2ccnc3[nH]cc(C#N)c23)cc(F)c1[C@@]1(O)[C@H](C)CN(C(=O)CN)C[C@@H]1C]

### 4-(3-fluoro-4-((3S,4s,5R)-1-glycyl-4-hydroxy-3,5-dimethylpiperidin-4-yl)-5-methylphenyl)-1H-pyrrolo[2,3-b]pyridine-3-carbonitrile

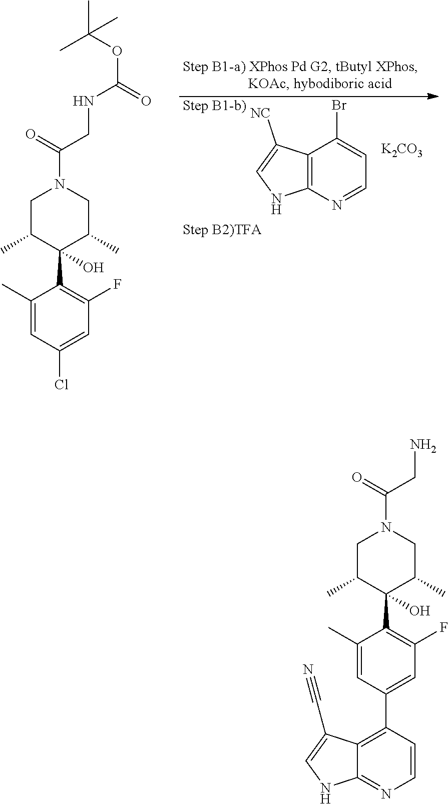

Step B1-a, B1-b: A 5 mL microwave vial with a stirbar was charged tert-butyl (2-((3S,4s,5R)-4-(4-chloro-2-fluoro-6-methylphenyl)-4-hydroxy-3,5-dimethylpiperidin-1-yl)-2-oxoethyl)carbamate (0.050 g, 0.121 mmol) followed by hypodiboric acid (0.033 g, 0.363 mmol), potassium acetate (0.036 g, 0.363 mmol), chloro(2-dicyclohexylphosphino-2′,4′,6′-triisopropyl-1,1′-biphenyl)[2-(2′-amino-1,1′-biphenyl)]palladium(II) (0.005 g, 0.001 mmol, Xphos Pd G2 catalyst) and 2-di-tert-butylphosphino-2′,4′,6′-triisopropylbiphenyl (0.005 g, 0.001 mmol, tert-butyl Xphos). The vial was sealed, evacuated, and backfilled with argon. Than ethanol (3.0 ml, previously degassed via argon bubbling) was added via syringe. The reaction was then heated to 80° C. for 30 minutes by microwave irradiation. The vial was allowed to cool. Potassium carbonate (0.050 g, 0.363 mmol) in water (1.0 ml, degassed by argon bubbling) was added to the vial via syringe. Then 4-bromo-1H-pyrrolo[2,3-b]pyridine-3-carbonitrile (0.027 g, 0.121 mmol) dissolved in 2.0 mL of ethanol (degassed by argon bubbling) was added to the vial via syringe. The reaction was then heated to 100° C. for a further 30 minutes via microwave irradiation. The vial was allowed to cool. The reaction was then extracted into ethyl acetate and washed with saturated sodium bicarbonate. The organic phase was taken, and the solvent removed by rotary evaporation. Purification by reverse phase medium pressure liquid chromatography (5% to 100% acetonitrile containing 0.1% trifluoroacetic acid) allowed for isolation of the intermediate in an acetonitrile-water mixture. This mixture was treated with aqueous saturated sodium bicarbonate and extracted with ethyl acetate. The organic phase was taken, and the solvent removed by rotary evaporation to allow for the intermediate product, which was immediately used in step B2.

Step B2: The intermediate product from step B1-a, B1-b was dissolved in dichloromethane (1.0 mL) and then treated with trifluoroacetic acid (1.0 mL). The reaction was stirred for 30 minutes. The solvent and trifluoroacetic acid were then removed by rotary evaporation. Purification by reverse phase high pressure column chromatography (50 to 100% acetonitrile in water, with 0.1% trifluoroacetic acid modifier) gave the desired product. ¹H NMR (400 MHz, DMSO-d₆) δ 12.97 (s, 1H), 8.49 (d, J=3.0 Hz, 1H), 8.42 (d, J 4.9 Hz, 1H), 7.98 (bs, 3H), 7.28 (d, J=4.9 Hz, 1H), 7.18 (d, J=12.6 Hz, 2H), 4.66 (s, 1H), 4.19 (dd, J=11.9, 3.9 Hz, 1H), 4.12-4.03 (n, 1H), 3.89-3.79 (m, 1H), 3.43-3.37 (m, 2H), 3.14 (t, J=12.5 Hz, 1H), 2.87-2.73 (m, 1H), 2.71 (s, 3H), 2.42-2.34 (m, 1H), 0.75 (d, J=6.8 Hz, 6H). LC-MS (m/z [M+H]+): 436.1Xx

### Compounds 2005-2020

Compounds 2005-2020 were prepared using an analogous method to that of Compound 2004 and are shown in Table 38.

TABLE 38

| Compound | Structure | IUPAC name | ¹H NMR | LCMS m/z |
|---|---|---|---|---|
| 2005 | 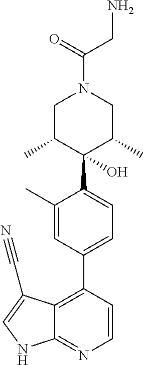 | 4-(4-((3S,4s,5R)-1- glycyl-4-hydroxy-3,5- dimethylpiperidin-4- yl)-3-methylphenyl)- 1H-pyrrolo[2,3-b] pyridine-3-carbonitrile | ¹H NMR (400 MHz, DMSO- d₆) δ 12.92 (s, 1H), 8.48 (d, J = 3.0 Hz, 1H), 8.45-8.38 (m, 1H), 7.99 (bs, 3H), 7.85 (d, J = 8.2 Hz, 1H), 7.46- 7.38 (m, 1H), 7.32-7.23 (m, 2H), 4.87 (s, 1H), 4.29-4.17 (m, 1H), 4.14-3.97 (m, 1H), 3.93-3.80 (m, 1H), 3.47- 3.44 (m, 2H), 3.18 (t, J = 11.7 Hz, 1H), 2.82 (t, J = 12.0 Hz, 1H), 2.67 (s, H), 2.48 (s, 2H), 2.43-2.30 (m, 1H), 0.70 (d, J = 6.7 Hz, 2H), 0.60 (dd, J = 6.8, 4.9 Hz, 4H). | [M + H]⁺: 511.2 |
| 2006 | 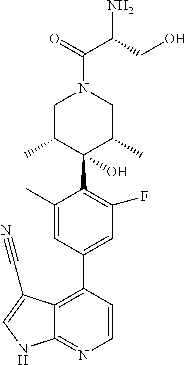 [SMILES: Cc1cc(-c2ccnc3[nH]cc(C#N)c23)cc(F)c1[C@@]1(O)[C@H](C)CN(C(=O)[C@H](N)CO)C[C@@H]1C] | 4-(4-((3S,4R,5R)-1- (L-seryl)-4-hydroxy- 3,5-dimethylpiperidin- 4-yl)-3-fluoro-5-meth- ylphenyl)-1H-pyrrolo [2,3-b]pyridine-3- carbonitrile | | [M + H]⁺: 466.1 |
| 2007 | 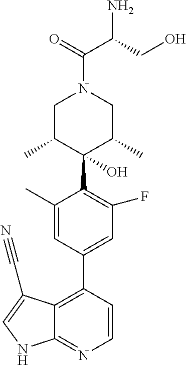 [SMILES: Cc1cc(-c2ccnc3[nH]cc(C#N)c23)cc(F)c1[C@@]1(O)[C@H](C)CN(C(=O)[C@H](N)CO)C[C@@H]1C] | 4-(4-((3S,4S,5R)-1- (D-seryl)-4-hydroxy- 3,5-dimethylpiperidin- 4-yl)-3-fluoro-5-meth- ylphenyl)-1H-pyrrolo [2,3-b]pyridine-3- carbonitrile | ¹H NMR (400 MHz, DMSO- d₆) δ 12.98 (s, 1H), 8.50 (d, J = 3.0 Hz, 1H), 8.44 (d, J = 4.9 Hz, 1H), 8.09 (bs, 3H), 7.29 (d, J = 4.9 Hz, 1H), 7.23-7.17 (m, 2H), 5.46 (d, J = 26.4 Hz, 1H), 4.68 (d, J = 4.6 Hz, 1H), 4.49 (d, J = 62.9 Hz, 1H), 4.21 (d, J = 12.5 Hz, 1H), 3.77-3.57 (m, 5H), 3.21 (t, J = 12.6 Hz, 1H), 2.85-2.73 (m, 1H), 2.72 (s, 3H), 2.46-2.26 (m, 1H), 1.23 (s, 1H), 0.80-0.72 (m, 6H). | [M + H]⁺: 466.2 |
| 2008 | 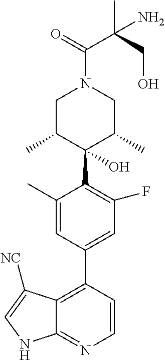 [SMILES: Cc1cc(-c2ccnc3[nH]cc(C#N)c23)cc(F)c1[C@@]1(O)[C@H](C)CN(C(=O)[C@](C)(N)CO)C[C@@H]1C] | 4-(4-((3S,4s,5R)-1-(2- amino-3-hydroxy-2- methylpropanoyl)-4- hydroxy-3,5-dimethyl- piperidin-4-yl)-3- fluoro-5-methylphen- yl)-1H-pyrrolo[2,3-b] pyridine-3-carbonitrile | ¹H NMR (400 MHz, DMSO- d₆) δ 13.05-12.70 (m, 1H), 8.51 (d, J = 3.1 Hz, 1H), 8.44 (d, J = 4.9 Hz, 1H), 8.03 (s, 3H), 7.29 (d, J = 4.9 Hz, 1H), 7.20 (d, J = 11.7 Hz, 2H), 5.73 (s, 1H), 4.69 (s, 1H), 4.01-3.87 (m, 3H), 3.68 (d, J = 11.9 Hz, 1H), 3.02-2.90 (m, 1H), 2.73 (s, 3H), 2.49- 2.38 (m, 1H), 1.50 (s, 3H), 0.78 (d, J = 6.8 Hz, 6H). | [M + H]⁺: 480.3 |
| 2009 | 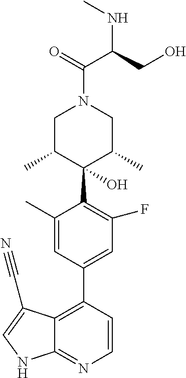 [SMILES: CN[C@@H](CO)C(=O)N1C[C@@H](C)[C@](O)(c2c(C)cc(-c3ccnc4[nH]cc(C#N)c34)cc2F)[C@@H](C)C1] | 4-(3-fluoro-4- ((3S,4R,5R)-4- hydroxy-3,5-dimethyl- 1-(methyl-L-seryl)- piperidin-4-yl)-5-meth- ylphenyl)-1H-pyrrolo [2,3-b]pyridine-3- carbonitrile | ¹H NMR (400 MHz, DMSO- d₆) δ 13.06-12.84 (m, 1H), 8.76 (bs, 2H), 8.50 (dd, J = 3.1, 1.4 Hz, 1H), 8.44 (d, J = 4.9 Hz, 1H), 7.29 (dd, J = 5.0, 1.1 Hz, 1H), 7.23-7.16 (m, 2H), 5.59 (d, J = 29.2 Hz, 1H), 4.70 (d, J = 6.8 Hz, 1H), 4.66-4.46 (m, 1H), 4.22 (dd, J = 12.9, 4.1 Hz, 1H), 3.80 (d, J = 12.0 Hz, 1H), 3.74-3.65 (m, 1H), 3.59 (t, J = 11.6 Hz, 1H), 3.22 (t, J = 12.7 Hz, 1H), 2.88-2.76 (m, 1H), 2.72 (s, 3H), 2.59-2.52 (m, 3H), 2.46-2.29 (m, 1H), 0.77 (dd, J = 6.7, 3.1 Hz, 6H). | [M + H]⁺: 480.0 |
| 2010 | 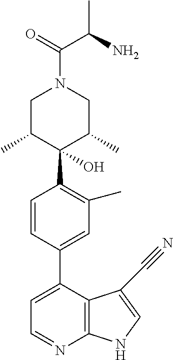 [SMILES: Cc1cc(-c2ccnc3[nH]cc(C#N)c23)ccc1[C@@]1(O)[C@H](C)CN(C(=O)[C@@H](C)N)C[C@@H]1C] | 4-(4-((3S,4S,5R)-1- (D-alanyl)-4-hydroxy- 3,5-dimethylpiperidin- 4-yl)-3-methylphen- yl)-1H-pyrrolo[2,3-b] pyridine-3-carbonitrile | ¹H NMR (400 MHz, DMSO) δ 12.94 (s, 1H), 8.49 (d, J = 2.9 Hz, 1H), 8.43 (dd, J = 5.0, 2.9 Hz, 1H), 8.09 (s, 3H), 7.86 (d, J = 8.2 Hz, 1H), 7.46-7.35 (m, 2H), 7.30-7.20 (m, 1H), 4.91 (s, 1H), 4.61- 4.37 (m, 2H), 4.29-4.17 (m, 1H), 3.71-3.54 (m, 1H), 3.29- 3.16 (m, 1H), 2.87-2.76 (m, 1H), 2.69 (s, 1H), 2.43-2.3 (m, 1H), 1.42-2.30 (m, 3H), 0.77-0.68 (m, 2H), 0.66- 0.58 (m, 4H). | [M + H]⁺: 432.2 |
| 2011 | 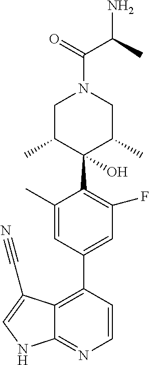 [SMILES: Cc1cc(-c2ccnc3[nH]cc(C#N)c23)cc(F)c1[C@@]1(O)[C@H](C)CN(C(=O)[C@H](C)N)C[C@@H]1C] | 4-(4-((3S,4R,5R)-1- (L-alanyl)-4-hydroxy- 3,5-dimethylpiperidin- 4-yl)-3-fluoro-5-meth- ylphenyl)-1H-pyrrolo [2,3-b]pyridine-3- carbonitrile | | [M + H]⁺: 450.2 |
| 2012 | 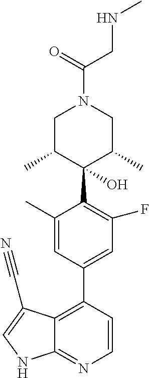 [SMILES: CNCC(=O)N1C[C@@H](C)[C@](O)(c2c(C)cc(-c3ccnc4[nH]cc(C#N)c34)cc2F)[C@@H](C)C1] | 4-(3-fluoro-4- ((3S,4s,5R)-4-hydroxy- 3,5-dimethyl-1-(meth- ylglycyl)piperidin-4- yl)-5-methylphenyl)- 1H-pyrrolo[2,3-b]pyr- idine-3-carbonitrile | ¹H NMR (400 MHz, DMSO- d₆) δ 12.98 (bs, 1H), 8.65 (s, 2H), 8.51 (d, J = 3.1 Hz, 1H), 8.44 (d, J = 4.9 Hz, 1H), 7.30 (d, J = 4.9 Hz, 1H), 7.23-7.17 (m, 2H), 4.70 (s, 1H), 4.31- 4.15 (m, 2H), 4.12-3.96 (m, 1H), 3.37 (d, J = 13.3 Hz, 1H), 3.30-3.07 (m, 1H), 2.80 (t, J = 11.8 Hz, 1H), 2.72 (s, 3H), 2.74 (t, J = 5.4 Hz, 3H), 2.55-2.52 (m, 1H), 2.44-2.35 (m, 1H), 0.77 (d, J = 6.8 Hz, 6H). | [M + H]⁺: 450.0 |
| 2013 | 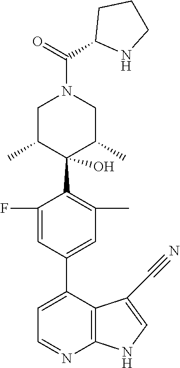 [SMILES: Cc1cc(-c2ccnc3[nH]cc(C#N)c23)cc(F)c1[C@@]1(O)[C@H](C)CN(C(=O)[C@@H]2CCCN2)C[C@@H]1C] | 4-(4-((3S,4S,5R)-1- (L-prolyl)-4-hydroxy- 3,5-dimethylpiperidin- 4-yl)-3-fluoro-5-meth- ylphenyl)-1H-pyrrolo [2,3-b]pyridine-3- carbonitrile | ¹H NMR (400 MHz, DMSO) δ 12.99 (s, 1H), 9.28 (s, 1H), 8.51 (dd, J = 3.1, 0.9 Hz, 1H), 8.44 (d, J = 4.9 Hz, 1H), 7.29 (dd, J = 4.9, 2.1 Hz, 1H), 7.21 (s, 1H), 7.25-7.16 (m, 1H), 4.75 (m, 2H), 4.62 (t, J = 7.2 Hz, 1H), 4.19 (d, J = 12.3 Hz, 1H), 3.32-3.12 (m, 2H), 2.85 (t, J = 12.5 Hz, 1H), 2.72 (s, 3H), 2.49-2.34 (m, 1H), 2.03- 1.84 (m, 2H), 1.83-1.67 (m, 1H), 0.83-0.74 (m, 6H). | [M + H]⁺: 476.2 |
| 2014 | 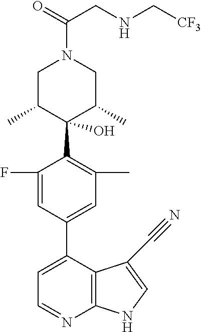 [SMILES: Cc1cc(-c2ccnc3[nH]cc(C#N)c23)cc(F)c1[C@@]1(O)[C@H](C)CN(C(=O)CNCC(F)(F)F)C[C@@H]1C] | 4-(3-fluoro-4- ((3S,4s,5R)-4-hydroxy- 3,5-dimethyl-1-((2,2,2- trifluoroethyl)glycyl)- piperidin-4-yl)-5-meth- ylphenyl)-1H-pyrrolo [2,3-b]pyridine-3- carbonitrile | ¹H NMR (400 MHz, DMSO- d₆) δ 12.97 (s, 1H), 8.50 (d, J = 2.9 Hz, 1H), 8.43 (d, J = 4.9 Hz, 1H), 7.29 (d, J = 4.9 Hz, 1H), 7.24-7.14 (m, 2H), 4.65 (s, 1H), 4.20 (d, J = 12.9 Hz, 1H), 4.01 (d, J = 15.2 Hz, 1H), 3.83 (d, J = 16.4 Hz, 1H), 3.67 (d, J = 10.0 Hz, 1H), 3.40 (d, J = 13.4 Hz, 1H), 3.15 (t, J = 12.5 Hz, 1H), 2.81-2.73 (m, 1H), 2.72 (s, 3H), 2.44-2.31 (m, 1H), 0.76 (d, J = 6.8 Hz, 6H). | [M + H]⁺: 518.2 |
| 2015 | 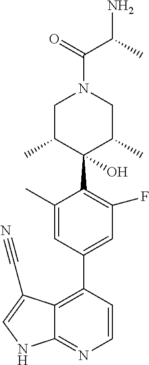 [SMILES: Cc1cc(-c2ccnc3[nH]cc(C#N)c23)cc(F)c1[C@@]1(O)[C@H](C)CN(C(=O)[C@@H](C)N)C[C@@H]1C] | 4-(4-((3S,4S,5R)-1- (D-alanyl)-4-hydroxy- 3,5-dimethylpiperidin- 4-yl)-3-fluoro-5-meth- ylphenyl)-1H-pyrrolo [2,3-b]pyridine-3- carbonitrile | ¹H NMR (400 MHz, DMSO- d₆) δ 13.09-12.80 (m, 1H), 8.50 (dd, J = 3.1, 1.3 Hz, 1H), 8.43 (d, J = 4.9 Hz, 1H), 8.11-8.00 (m, 3H), 7.28 (dd, J = 4.9, 1.3 Hz, 1H), 7.22-7.15 (m, 2H), 4.69 (d, J = 11.9 Hz, 1H), 4.60-4.38 (m, 1H), 4.20 (d, J = 12.9 Hz, 1H), 3.59 (t, J = 12.3 Hz, 1H), 3.26-3.12 (m, 1H), 2.79 (t, J = 12.1 Hz, 1H), 2.72 (s, 3H), 2.45-2.29 (m, 1H), 1.32 (dd, J = 12.0, 6.8 Hz, 3H), 0.81-0.73 (m, 6H). | [M + H]⁺: 450.0 |
| 2016 | 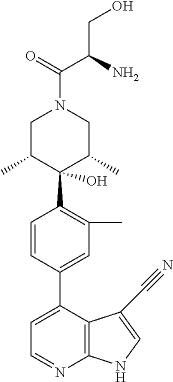 [SMILES: Cc1cc(-c2ccnc3[nH]cc(C#N)c23)ccc1[C@@]1(O)[C@H](C)CN(C(=O)[C@H](N)CO)C[C@@H]1C] | 4-(4-((3S,4S,5R)-1- (D-seryl)-4-hydroxy- 3,5-dimethylpiperidin- 4-yl)-3-methylphen- yl)-1H-pyrrolo[2,3-b] pyridine-3-carbonitrile | | [M + H]⁺: 448.2 |
| 2017 | 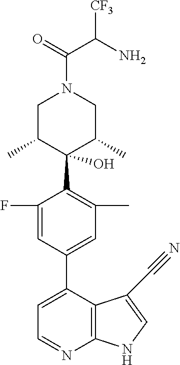 [SMILES: Cc1cc(-c2ccnc3[nH]cc(C#N)c23)cc(F)c1[C@@]1(O)[C@H](C)CN(C(=O)C(N)C(F)(F)F)C[C@@H]1C] | 4-(4-((3S,4s,5R)-1- (2-amino-3,3,3- trifluoropropanoyl)-4- hydroxy-3,5-dimethyl- piperidin-4-yl)-3- fluoro-5-methylphen- yl)-1H-pyrrolo[2,3-b] pyridine-3-carbonitrile | ¹H NMR (400 MHz, Methanol-d₄) δ 8.58 (dd, J = 5.7, 2.5 Hz, 1H), 8.43 (d, J = 4.4 Hz, 1H), 7.59 (t, J = 5.8 Hz, 1H), 7.32 (d, J = 2.5 Hz, 1H), 7.27 (ddd, J = 13.7, 5.7, 1.9 Hz, 1H), 5.76 (dq, J = 54.7, 6.8 Hz, 1H), 4.43- 4.24 (m, 1H), 3.79 (t, J = 9.6 Hz, 1H), 3.42 (dd, J = 13.9, 11.4 Hz, 1H), 3.02 (dddd, J = 12.4, 9.2, 6.5, 2.7 Hz, 1H), 2.81 (s, 3H), 2.75-2.49 (m, 1H), 0.87 (td, J = 6.7, 2.7 Hz, 6H). | [M + H]⁺: 504.0 |
| 2018 | 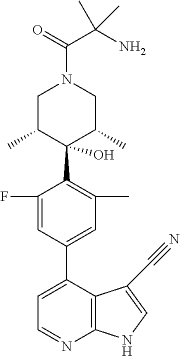 [SMILES: Cc1cc(-c2ccnc3[nH]cc(C#N)c23)cc(F)c1[C@@]1(O)[C@H](C)CN(C(=O)C(C)(C)N)C[C@@H]1C] | 4-(4-((3S,4s,5R)-1- (2-amino-2-methyl- propanoyl)-4-hydroxy- 3,5-dimethylpiperidin- 4-yl)-3-fluoro-5-meth- ylphenyl)-1H-pyrrolo [2,3-b]pyridine-3- carbonitrile | ¹H NMR (400 MHz, DMSO- d₆) δ 8.51 (s, 1H), 8.44 (d, J = 4.9 Hz, 1H), 7.29 (d, J = 4.9 Hz, 1H), 7.19 (d, J = 12.1 Hz, 2H), 4.64 (s, 1H), 4.25- 4.09 (m, 2H), 3.11-2.91 (m, 2H), 2.72 (s, 3H), 2.47-2.37 (m, 2H), 1.49 (s, 6H), 0.77 (d, J = 6.8 Hz, 6H). | [M + H]⁺: 464.1 |
| 2019 | 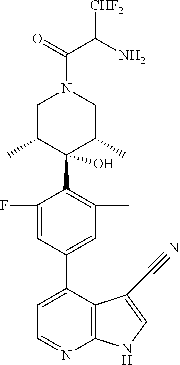 [SMILES: Cc1cc(-c2ccnc3[nH]cc(C#N)c23)cc(F)c1[C@@]1(O)[C@H](C)CN(C(=O)C(N)C(F)F)C[C@@H]1C] | 4-(4-((3S,4s,5R)-1-(2- amino-3,3-difluoro- propanoyl)-4-hydroxy- 3,5-dimethylpiperidin- 4-yl)-3-fluoro-5-meth- ylphenyl)-1H-pyrrolo [2,3-b]pyridine-3- carbonitrile | ¹H NMR (400 MHz, DMSO- d₆) δ 12.99 (s, 1H), 8.61 (s, 3H), 8.60-8.40 (m, 2H), 7.30 (dd, J = 4.9, 1.3 Hz, 1H), 7.21 (d, J = 9.9 Hz, 2H), 6.49 (t, J = 52.9 Hz, 1H), 5.44- 5.30 (m, 1H), 5.21-5.07 (m, 1H), 4.73 (d, J = 7.5 Hz, 1H), 4.24 (d, J = 12.7 Hz, 1H), 3.83-3.71 (m, 1H), 3.70-3.55 (m, 1H), 3.22-3.04 (m, 1H), 2.93-2.79 (m, 1H), 2.73 (s, 3H), 2.54 (s, 3H), 2.47-2.34 (m, 2H), 0.78 (d, J = 6.7 Hz, 6H). | [M + H]⁺: 486.2 |
| 2020 | 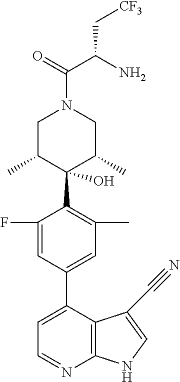 [SMILES: Cc1cc(-c2ccnc3[nH]cc(C#N)c23)cc(F)c1[C@@]1(O)[C@H](C)CN(C(=O)[C@@H](N)CC(F)(F)F)C[C@@H]1C] | 4-(4-((3S,4R,5R)-1- ((S)-2-amino-4,4,4- trifluorobutanoyl)-4- hydroxy-3,5-dimethyl- piperidin-4-yl)-3- fluoro-5-methylphen- yl)-1H-pyrrolo[2,3-b] pyridine-3-carbonitrile | ¹H NMR (400 MHz, Methanol-d₄) δ 8.51 (dd, J = 5.3, 2.5 Hz, 1H), 8.37-8.29 (m, 1H), 7.44 (dd, J = 6.7, 5.3 Hz, 1H), 7.30 (d, J = 3.5 Hz, 1H), 7.23 (dd, J = 13.7, 7.8 Hz, 1H), 4.98 (dd, J = 8.1, 4.2 Hz, 1H), 4.93-4.87 (m, 1H), 4.35 (dd, J = 13.1, 4.4 Hz, 1H), 3.56-3.35 (m, 2H), 3.03-2.83 (m, 2H), 2.82 (s, 3H), 2.77-2.55 (m, 2H), 0.88 (dd, J = 10.0, 6.9 Hz, 6H). | [M + H]⁺: 518.0 |

### Synthesis of Compounds of General Formula N4

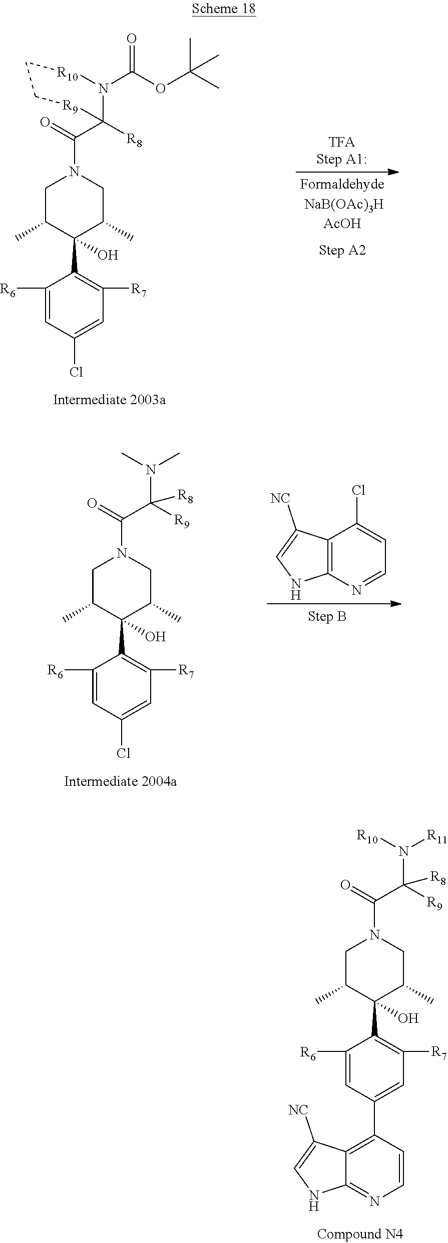

### Synthesis of Intermediates and Compounds of Scheme 18

### Intermediate 2004a-1 (Scheme 18) (R)-1-((3S,4S,5R)-4-(4-chloro-2-fluoro-6-methylphenyl)-4-hydroxy-3,5-dimethylpiperidin-1-yl)-2-(dimethylamino)-3-hydroxypropan-1-one

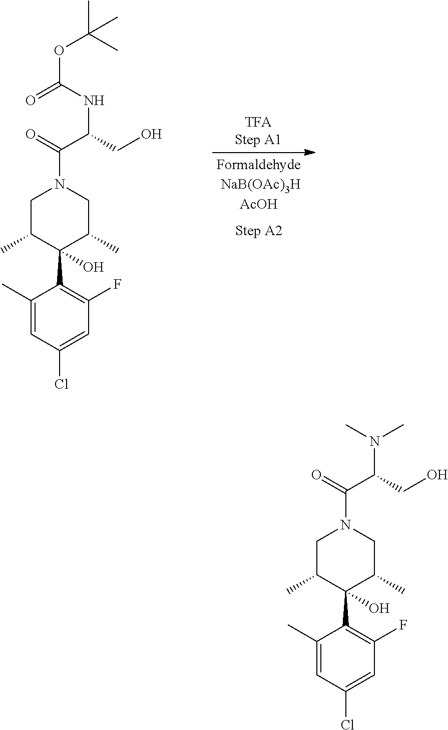 [SMILES: Cc1cc(Cl)cc(F)c1[C@@]1(O)[C@H](C)CN(C(=O)[C@@H](CO)NC(=O)OC(C)(C)C)C[C@@H]1C ; Cc1cc(Cl)cc(F)c1[C@@]1(O)[C@H](C)CN(C(=O)[C@@H](CO)N(C)C)C[C@@H]1C]

Step A1: A vial containing a stirbar was charged with tert-butyl ((R)-1-((3S,4S,5R)-4-(4-chloro-2-fluoro-6-methylphenyl)-4-hydroxy-3,5-dimethylpiperidin-1-yl)-3-hydroxy-1-oxopropan-2-yl)carbamate (0.070 g, 0.153 mmol). To this was added dichloromethane (1.0 mL) followed by trifluoroacetic acid (1.48 g, 12.98 mmol, 1.0 mL). The reaction was then stirred for 30 minutes at 20° C. The solvent and trifluoroacetic acid were then removed by rotary evaporation. The residue was treated with aqueous saturated sodium bicarbonate and then extracted into ethyl acetate. The organic phase was taken, and the solvent removed by rotary evaporation to give the crude intermediate which was used without further purification.

Step A2: A vial containing a stirbar was charged with (R)-2-amino-1-((3S,4S,5R)-4-(4-chloro-2-fluoro-6-methylphenyl)-4-hydroxy-3,5-dimethylpiperidin-1-yl)-3-hydroxypropan-1-one (0.055 g, 0.153 mmol). To this was added tetrahydrofuran (5.0 mL), followed by formaldehyde (0.062 g, 0.766 mmol, 37% solution in water, 0.057 mL). Then acetic acid (0.0092 g, 0.153 mmol) was added. The reaction was stirred at 20° C. and placed in a 20° C. water bath to regulate the temperature. Then sodium triacetoxyborohydride (0.162 g, 0.766 mmol) was added as a solid portionwise. The vial was capped and the reaction allowed to proceed for 3 hours. The reaction was then quenched by being poured into aqueous saturated sodium bicarbonate. The mixture was extracted with ethyl acetate, the organic phase taken, and the solvent removed by rotary evaporation to give the product which was used without further purification. LC-MS (m/z [M+H]+): 387.2.

### Intermediates 2004a-2 and 2004a-3 (Scheme 18)

Intermediates 4a-2 and 4a-3 were prepared using an analogous method to that of Intermediate 4a-1 and are shown in Table 39.

TABLE 39

| Compound | Structure | IUPAC name | LCMS m/z |
|---|---|---|---|
| 2004a-2 | 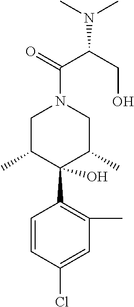 | (R)-1-((3S,4S,5R)-4-(4- chloro-2-methylphenyl)- 4-hydroxy-3,5-dimeth- ylpiperidin-1-y1)-2- (dimethylamino)-3- hydroxypropan-1-one | [M + H]⁺: 369.0 |
| 2004a-3 | 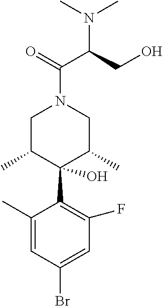 [SMILES: Cc1cc(Br)cc(F)c1[C@@]1(O)[C@H](C)CN(C(=O)[C@H](CO)N(C)C)C[C@@H]1C] | (S)-1-((3S,4R,5R)-4-(4- bromo-2-fluoro-6-meth- ylphenyl)-4-hydroxy- 3,5-dimethylpiperidin-1- yl)-2-(dimethylamino)- 3-hydroxypropan-1-one | [M + H]⁺: 432.9 |

### Compound 2021

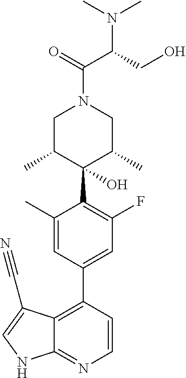 [SMILES: Cc1cc(-c2ccnc3[nH]cc(C#N)c23)cc(F)c1[C@@]1(O)[C@H](C)CN(C(=O)[C@@H](CO)N(C)C)C[C@@H]1C]

### 4-(4-((3S,4S,5R)-1-(dimethyl-D-seryl)-4-hydroxy-3,5-dimethylpiperidin-4-yl)-3-fluoro-5-methylphenyl)-1H-pyrrolo[2,3-b]pyridine-3-carbonitrile

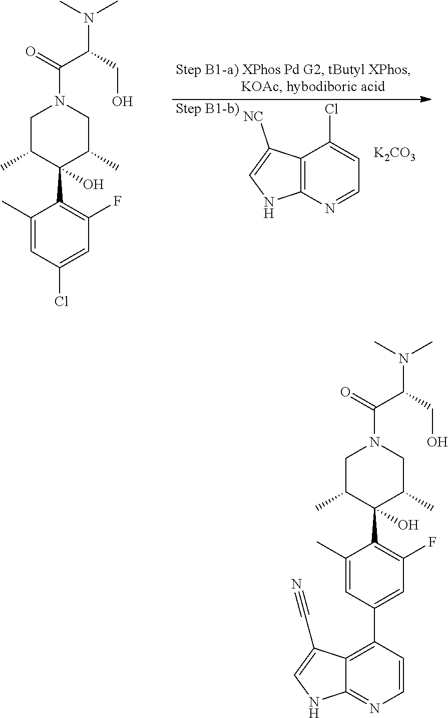

Step B1-a, B1-b: A 5 mL microwave vial with a stirbar was charged with (R)-1-((3S,4S,5R)-4-(4-chloro-2-fluoro-6-methylphenyl)-4-hydroxy-3,5-dimethylpiperidin-1-yl)-2-(dimethylamino)-3-hydroxypropan-1-one (0.035 g, 0.090 mmol), followed by hypodiboric acid (0.024 g, 0.271 mmol), potassium acetate (0.027 g, 0.271 mmol), chloro(2-dicyclohexylphosphino-2′,4′,6′-triisopropyl-1,1′-biphenyl)[2-(2′-amino-1,1′-biphenyl)]palladium(II) (0.004 g, 0.0004 mmol, Xphos Pd G2 catalyst) and 2-di-tert-butylphosphino-2′,4′,6′-triisopropylbiphenyl (0.002 g, 0.0004 mmol, tert-butyl Xphos). The vial was sealed, evacuated, and backfilled with argon. Than ethanol (3.0 ml, previously degassed via argon bubbling) was added via syringe. The reaction was then heated to 80° C. for 30 minutes by microwave irradiation. The vial was allowed to cool. Potassium carbonate (0.038 g, 0.271 mmol) in water (1.0 ml, degassed by argon bubbling) was added to the vial via syringe. Then 4-chloro-1H-pyrrolo[2,3-b]pyridine-3-carbonitrile (0.021 g, 0.118 mmol) dissolved in 2.0 mL of ethanol (degassed by argon bubbling) was added to the vial via syringe. The reaction was then heated to 100° C. for a further 30 minutes via microwave irradiation. The vial was allowed to cool. The reaction was then extracted into ethyl acetate and washed with saturated sodium bicarbonate. The organic phase was taken, and the solvent removed by rotary evaporation. Purification by reverse phase high pressure column chromatography (5% to 100% acetonitrile in water, with 0.1% trifluoroacetic acid modifier) gave the desired product as the trifluoroacetate salt. ¹H NMR (400 MHz, DMSO-d₆) δ 12.99 (s, 1H), 8.52 (dd, J=3.1, 1.4 Hz, 1H), 8.45 (d, J=4.8 Hz, 1H), 7.31 (d, J=4.8 Hz, 1H), 7.25-7.18 (m, 2H), 5.73-5.52 (m, 1H), 4.71 (d, J=8.5 Hz, 1H), 4.23 (d, J=12.3 Hz, 1H), 3.92-3.77 (m, 1H), 3.74-3.15 (m, 7H), 2.89-2.76 (m, 3H), 2.73 (s, 3H), 2.62-2.53 (m, 2H), 2.08-1.91 (m, 1H), 0.84-0.68 (m, 6H). LC-MS (m/z [M+H]+). 494.3Xx

### Compounds 2022-2023

Compounds 22-23 were prepared using an analogous method to that of Compound 21 and are shown in Table 40.

TABLE 40

| Compound | Structure | IUPAC name | ¹H NMR | LCMS m/z |
|---|---|---|---|---|
| 2022 | 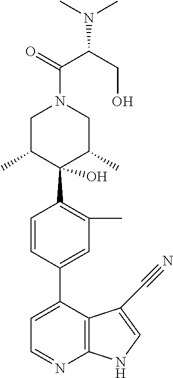 [SMILES: Cc1cc(-c2ccnc3[nH]cc(C#N)c23)ccc1[C@@]1(O)[C@H](C)CN(C(=O)[C@@H](CO)N(C)C)C[C@@H]1C] | 4-(4-((3S,4S,5R)-1- (dimethyl-D-seryl)-4- hydroxy-3,5-dimeth- ylpiperidin-4-yl)-3- methylphenyl)-1H- pyrrolo[2,3-b] pyridine-3-carbonitrile | ¹H NMR (400 MHz, DMSO) δ 12.94 (s, 1H), 9.54 (s, 1H), 8.48 (d, J = 3.0 Hz, 1H), 8.43 (dd, J = 4.9, 2.7 Hz, 1H), 7.85 (d, J = 8.1 Hz, 1H), 7.54-7.23 (m, 3H), 5.73-5.56 (m, 1H), 4.88 (d, J = 29.5 Hz, 1H), 4.68 (m, 1H), 4.51 (d, J = 26.6 Hz, 1H), 4.27 (s, 1H), 3.95-3.75 (m, 2H), 3.34- 3.07 (m, 1H), 2.93-2.80 (m, 6H), 2.76 (d, J = 4.4 Hz, 1H), 2.69 (s, 1H), 2.22-2.07 (m, 1H), 0.76-0.66 (m, 2H), 0.65- 0.54 (m, 4H). | [M + H]⁺: 476.3 |
| 2023 | 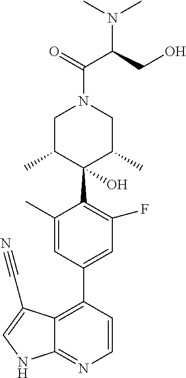 [SMILES: Cc1cc(-c2ccnc3[nH]cc(C#N)c23)cc(F)c1[C@@]1(O)[C@H](C)CN(C(=O)[C@H](CO)N(C)C)C[C@@H]1C] | 4-(4-((3S,4R,5R)-1- (dimethyl-L-seryl)-4- hydroxy-3,5-dimeth- ylpiperidin-4-yl)-3- fluoro-5-methyl- phenyl)-1H-pyrrolo [2,3-b]pyridine-3- carbonitrile | ¹H NMR (400 MHz, DMSO- d₆) δ 12.98 (s, 1H), 9.55 (s, 1H), 8.50 (dd, J = 3.1, 1.4 Hz, 1H), 8.44 (d, J = 4.9 Hz, 1H), 7.29 (dd, J = 4.9, 1.7 Hz, 1H), 7.25-7.17 (m, 2H), 5.67 (d, J = 33.1 Hz, 1H), 4.75-4.56 (m, 2H), 4.27-4.17 (m, 1H), 3.84 (q, J = 13.7 Hz, 2H), 3.75-3.52 (m, 1H), 3.31-3.17 (m, 1H), 2.86 (d, J = 4.7 Hz, 3H), 2.78 (dd, J = 17.4, 4.5 Hz, 2H), 2.72 (s, 3H), 2.59- 2.51 (m, 1H), 2.47-2.28 (m, 1H), 2.03-1.96 (m, 1H), 0.78 (t, J = 6.2 Hz, 6H). | [M + H]⁺: 494.3 |

### Compound 2024

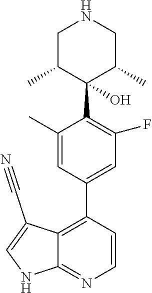 [SMILES: Cc1cc(-c2ccnc3[nH]cc(C#N)c23)cc(F)c1[C@@]1(O)[C@H](C)CNC[C@@H]1C]

### 4-(3-fluoro-4-((3S,4s,5R)-4-hydroxy-3,5-dimethylpiperidin-4-yl)-5-methylphenyl)-1H-pyrrolo[2,3-b]pyridine-3-carbonitrile

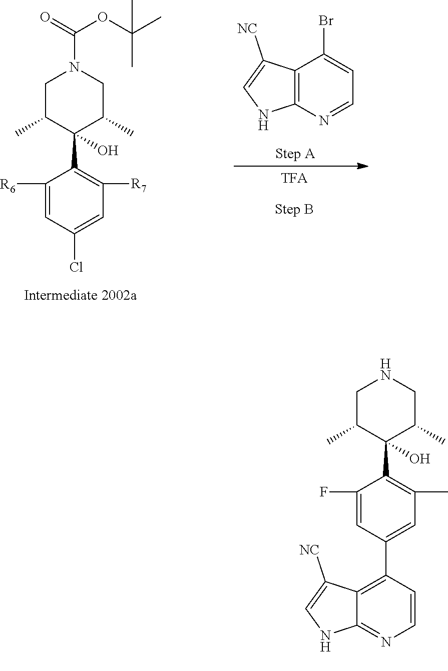

Step A: A 5 mL microwave vial with a stirbar was charged with tert-butyl (3S,4s,5R)-4-(4-chloro-2-fluoro-6-methylphenyl)-4-hydroxy-3,5-dimethylpiperidine-1-carboxylate (0.050 g, 0.134 mmol) followed by hypodiboric acid (0.036 g, 0.403 mmol), potassium acetate (0.040 g, 0.403 mmol), chloro(2-dicyclohexylphosphino-2′,4′,6′-triisopropyl-1,1′-biphenyl)[2-(2′-amino-1,1′-biphenyl)]palladium(II) (0.005 g, 0.001 mmol, Xphos Pd G2 catalyst) and 2-di-tert-butylphosphino-2′,4′,6′-triisopropylbiphenyl (0.006 g, 0.001 mmol, tert-butyl Xphos). The vial was sealed, evacuated, and backfilled with argon. Than ethanol (3.0 ml, previously degassed via argon bubbling) was added via syringe. The reaction was then heated to 80° C. for 30 minutes by microwave irradiation. The vial was allowed to cool. Potassium carbonate (0.056 g, 0.4.03 mmol) in water (1.0 ml, degassed by argon bubbling) was added to the vial via syringe. Then 4-bromo-1H-pyrrolo[2,3-b]pyridine-3-carbonitrile (0.030 g, 0.134 mmol) dissolved in 2.0 mL of 1:1 ethanol:tetrahydrofuran (degassed by argon bubbling) was added to the vial via syringe. The reaction was then heated to 100° C. for a further 30 minutes via microwave irradiation. The vial was allowed to cool. The reaction was then extracted into ethyl acetate and washed with saturated sodium bicarbonate. The organic phase was taken, and the solvent removed by rotary evaporation to give the crude product which was immediately used in step B.

Step B: The intermediate product from step A was dissolved in dichloromethane (1.0 mL) and then treated with trifluoroacetic acid (1.0 mL). The reaction was stirred for 30 minutes. The solvent and trifluoroacetic acid were then removed by rotary evaporation. Purification by reverse phase high pressure column chromatography (5% to 100% acetonitrile in water, with 0.1% trifluoroacetic acid modifier) gave the desired product. ¹H NMR (400 MHz, DMSO-d₆) δ 13.00 (s, 1H), 8.65 (bs, 3H), 8.52 (d, J=3.1 Hz, 1H), 8.46 (d, J=4.9 Hz, 1H), 7.31 (d, J=4.9 Hz, 1H), 7.29-7.21 (m, 2H), 4.94 (s, 1H), 3.12-3.05 (m, 2H), 3.03-2.91 (m, 2H), 2.77-2.66 (m, 2H), 2.71 (s, 3H), 0.78 (d, J=6.9 Hz, 6H). LC-MS (m/z [M+H]+): 379.2

### Example 11: Nitrile LATS Inhibitors: Biological Evaluation

Biochemical kinase inhibitory data was obtained for various exemplary compounds prepared according to the above in accordance with the protocols described in Examples 4 and 8. The IC₅₀ values are reported in Table 41. All biological activities are IC₅₀, in nM.

TABLE 41

| | | | | | LATS1 | AKT |
|---|---|---|---|---|---|---|
| Compound | Structure | LATS1 | LATS2 | AKT | (Cellular) | (Cellular) |
| 2001 | 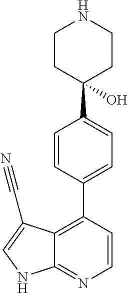 | 3.3 | 3.3 | 278 | | |
| 2002 | 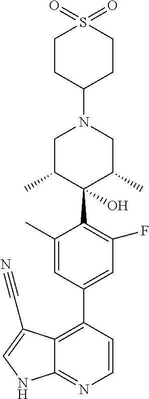 [SMILES: Cc1cc(-c2ccnc3[nH]cc(C#N)c23)cc(F)c1[C@@]1(O)[C@H](C)CN(C2CCS(=O)(=O)CC2)C[C@@H]1C] | 10.9 | 4.6 | 188 | 30.4 | 57900 |
| 2003 | 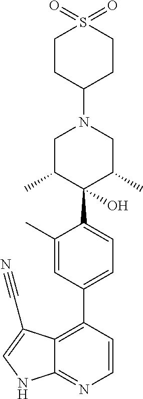 [SMILES: Cc1cc(-c2ccnc3[nH]cc(C#N)c23)ccc1[C@@]1(O)[C@H](C)CN(C2CCS(=O)(=O)CC2)C[C@@H]1C] | 5.6 | 2.6 | 12.1 | 15.0 | 28500 |
| 2004 | 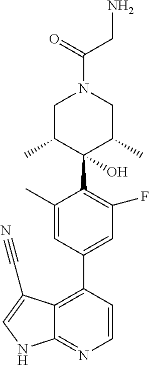 [SMILES: Cc1cc(-c2ccnc3[nH]cc(C#N)c23)cc(F)c1[C@@]1(O)[C@H](C)CN(C(=O)CN)C[C@@H]1C] | 6.7 | 2.0 | 118 | 2.5 | 51700 |
| 2005 | 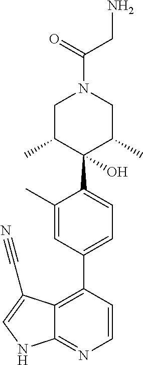 [SMILES: Cc1cc(-c2ccnc3[nH]cc(C#N)c23)ccc1[C@@]1(O)[C@H](C)CN(C(=O)CN)C[C@@H]1C] | 3.3 | 1.2 | 22.0 | 46.6 | 56800 |
| 2006 | 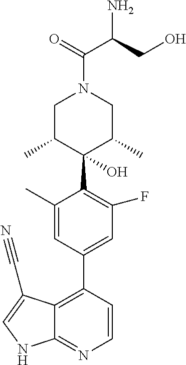 [SMILES: Cc1cc(-c2ccnc3[nH]cc(C#N)c23)cc(F)c1[C@@]1(O)[C@H](C)CN(C(=O)[C@@H](N)CO)C[C@@H]1C] | 3.6 | 1.4 | 126 | | |
| 2007 | 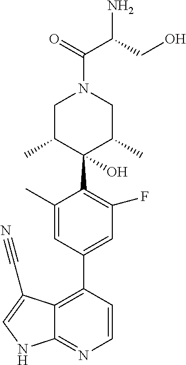 [SMILES: Cc1cc(-c2ccnc3[nH]cc(C#N)c23)cc(F)c1[C@@]1(O)[C@H](C)CN(C(=O)[C@H](N)CO)C[C@@H]1C] | 8.5 | 5.3 | 159 | <2.5 | >50000 |
| 2008 | 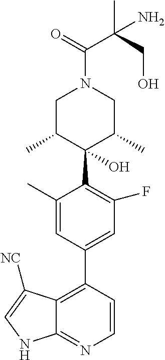 [SMILES: Cc1cc(-c2ccnc3[nH]cc(C#N)c23)cc(F)c1[C@@]1(O)[C@H](C)CN(C(=O)[C@](C)(N)CO)C[C@@H]1C] | 5.2 | 5.5 | 262 | <2.5 | >50000 |
| 2009 | 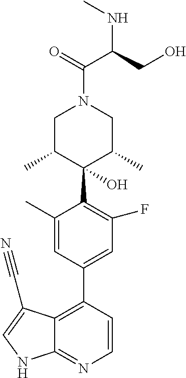 [SMILES: CN[C@@H](CO)C(=O)N1C[C@@H](C)[C@](O)(c2c(C)cc(-c3ccnc4[nH]cc(C#N)c34)cc2F)[C@@H](C)C1] | 4.8 | 2.5 | 244 | | |
| 2010 | 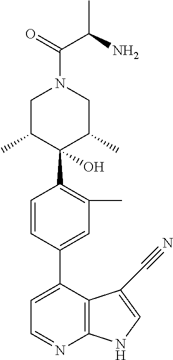 [SMILES: Cc1cc(-c2ccnc3[nH]cc(C#N)c23)ccc1[C@@]1(O)[C@H](C)CN(C(=O)[C@@H](C)N)C[C@@H]1C] | 13.4 | 8.5 | 89.4 | | |
| 2011 | 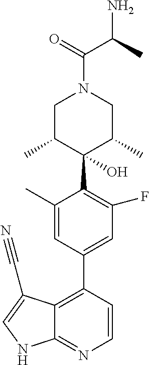 [SMILES: Cc1cc(-c2ccnc3[nH]cc(C#N)c23)cc(F)c1[C@@]1(O)[C@H](C)CN(C(=O)[C@H](C)N)C[C@@H]1C] | 25 | 16 | 1030 | <2.5 | >50000 |
| 2012 | 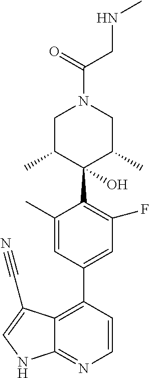 [SMILES: CNCC(=O)N1C[C@@H](C)[C@](O)(c2c(C)cc(-c3ccnc4[nH]cc(C#N)c34)cc2F)[C@@H](C)C1] | 5.0 | 2.9 | 349 | <2.5 | >50000 |
| 2013 | 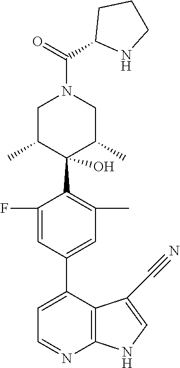 [SMILES: Cc1cc(-c2ccnc3[nH]cc(C#N)c23)cc(F)c1[C@@]1(O)[C@H](C)CN(C(=O)[C@@H]2CCCN2)C[C@@H]1C] | 5.7 | 2.9 | 330 | | |
| 2014 | 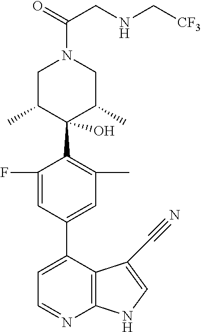 [SMILES: Cc1cc(-c2ccnc3[nH]cc(C#N)c23)cc(F)c1[C@@]1(O)[C@H](C)CN(C(=O)CNCC(F)(F)F)C[C@@H]1C] | 7.1 | 6.0 | 3010 | 139 | >50000 |
| 2015 | 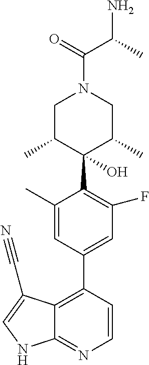 [SMILES: Cc1cc(-c2ccnc3[nH]cc(C#N)c23)cc(F)c1[C@@]1(O)[C@H](C)CN(C(=O)[C@@H](C)N)C[C@@H]1C] | 4.0 | 2.0 | 236 | <2.5 | >50000 |
| 2016 | 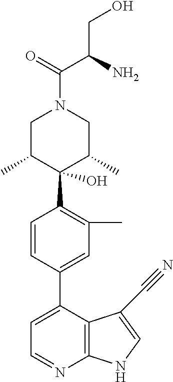 [SMILES: Cc1cc(-c2ccnc3[nH]cc(C#N)c23)ccc1[C@@]1(O)[C@H](C)CN(C(=O)[C@H](N)CO)C[C@@H]1C] | 9.2 | 6.4 | 48.8 | | |
| 2017 | 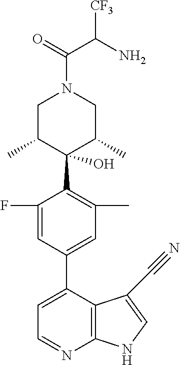 [SMILES: Cc1cc(-c2ccnc3[nH]cc(C#N)c23)cc(F)c1[C@@]1(O)[C@H](C)CN(C(=O)C(N)C(F)(F)F)C[C@@H]1C] | 8.2 | 6.9 | 2050 | <2.5 | >50000 |
| 2018 | 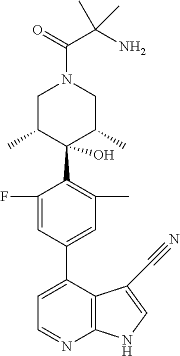 [SMILES: Cc1cc(-c2ccnc3[nH]cc(C#N)c23)cc(F)c1[C@@]1(O)[C@H](C)CN(C(=O)C(C)(C)N)C[C@@H]1C] | 11.1 | 7.2 | 610 | | |
| 2019 | 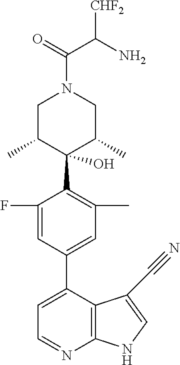 [SMILES: Cc1cc(-c2ccnc3[nH]cc(C#N)c23)cc(F)c1[C@@]1(O)[C@H](C)CN(C(=O)C(N)C(F)F)C[C@@H]1C] | 7.0 | 7.0 | 1220 | | |
| 2020 | 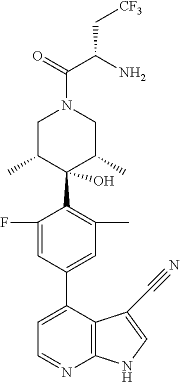 [SMILES: Cc1cc(-c2ccnc3[nH]cc(C#N)c23)cc(F)c1[C@@]1(O)[C@H](C)CN(C(=O)[C@@H](N)CC(F)(F)F)C[C@@H]1C] | | | | | |
| 2021 | 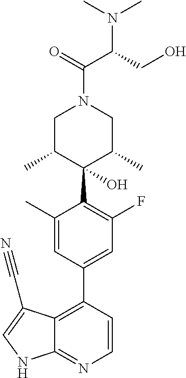 [SMILES: Cc1cc(-c2ccnc3[nH]cc(C#N)c23)cc(F)c1[C@@]1(O)[C@H](C)CN(C(=O)[C@@H](CO)N(C)C)C[C@@H]1C] | 5.1 | 3.4 | 223 | | |
| 2022 | 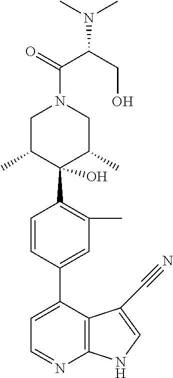 [SMILES: Cc1cc(-c2ccnc3[nH]cc(C#N)c23)ccc1[C@@]1(O)[C@H](C)CN(C(=O)[C@@H](CO)N(C)C)C[C@@H]1C] | 5.3 | 3.0 | 251 | | |
| 2023 | 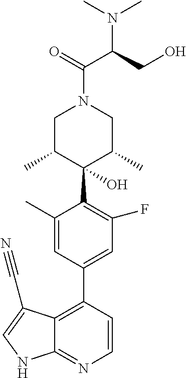 [SMILES: Cc1cc(-c2ccnc3[nH]cc(C#N)c23)cc(F)c1[C@@]1(O)[C@H](C)CN(C(=O)[C@H](CO)N(C)C)C[C@@H]1C] | 4.4 | 2.9 | 388 | <2.5 | >50000 |
| 2024 | 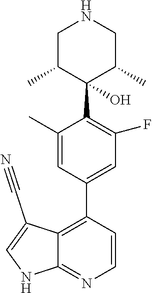 [SMILES: Cc1cc(-c2ccnc3[nH]cc(C#N)c23)cc(F)c1[C@@]1(O)[C@H](C)CNC[C@@H]1C] | 6.6 | 4.2 | 334 | 37.9 | >50000 |

1-126. (canceled)

127. A compound of Formula I 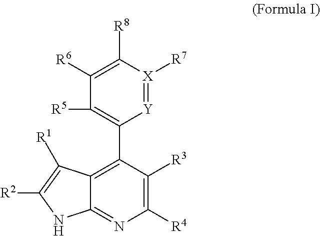
or a pharmaceutically acceptable salt thereof,
wherein
X and Y are independently C or N;
R¹ is —H, halo, —CN, —C₁-C₆ alkyl, —C₁-C₆ alkenyl, —C(O)O—C₁-C₆ alkyl, a 6-10 membered aryl ring, or a 5-10 membered heteroaryl ring containing 1, 2, or 3 heteroatoms, wherein the aryl or heteroaryl ring is optionally substituted with one or more substituents selected from halo, —C₁-C₆ alkyl, —O—C₁-C₆ alkyl, halogenated C₁-C₆ alkyl, and —C(O)NH₂;
R² is —H or —C₁-C₆ alkyl;
R³ is —H or halo;
R⁴ is —H, halo, —C₁-C₆ alkyl, —NR^{a}R^{b}, or a 5-10 membered heteroaryl ring containing 1 or 2 heteroatoms;
R⁵ is —H, halo, —CN, —C₁-C₆ alkyl, halogenated C₁-C₆ alkyl, or —O—C₁-C₆ alkyl, wherein —O—C₁-C₆ alkyl is optionally substituted with one or more halo groups;
R⁶ is —H, halo, —C₁-C₆ alkyl, —C₁-C₆ alkenyl, —C₁-C₆ alkynyl, —NR^{c}R^{d}, phenyl, pyridyl, or —O—C₁-C₆ alkyl, wherein —C₁-C₆ alkyl is optionally substituted with one or more halo groups, wherein —C₁-C₆ alkynyl is optionally substituted with one or more hydroxyl groups, and wherein phenyl is optionally substituted with one or more substituents selected from aminoalkyl and amide;
R⁷ is absent, —H, halo, or —C₁-C₆ alkyl;
R⁸ is 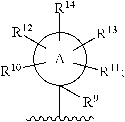
or R⁸ is 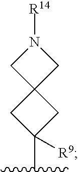
or R⁸ is —C(R^{e})(R^{f})N(R^{g})(R^{h}) or —C(O)NH₂;
or R⁸ and R⁶ together with the carbons to which they are attached form a 5- or 6-membered ring containing 1 or 2 heteroatoms and optionally a carbonyl group;
R^{a} and R^{b} are independently selected from —H and —C₁-C₆ alkyl;
R^{c} and R^{d} are independently selected from —H, —C₁-C₆ alkyl, and alkoxyalkyl;
R^{c} and R^{d} are independently selected from —H and —C₁-C₆ alkyl;
R^{g} is —H or —C₁-C₆ alkyl;
R^{h} is —H, —C₁-C₆ alkyl, —C(O)—C₁-C₆ alkyl, or arylalkyl, wherein the aryl group of arylalkyl is optionally substituted with halo or —OCF₃;
or R^{g} and R^{h} together with the carbons to which they are attached form a 5 or 6 membered heterocyclic ring;
A is a 4-6 membered heterocycloalkyl ring containing 1 nitrogen, optionally substituted at any position by one or more of R¹⁰, R¹¹, R¹², R¹³, and R¹⁴;
R⁹ is —H, —OH, —C₁-C₆ alkyl, halogenated C₁-C₆ alkyl, —COOH, —C(O)O—C₁-C₆ alkyl, or a 5- or 6-membered ring containing 1, 2, or 3 heteroatoms, wherein the ring is optionally substituted with one or more —C₁-C₆ alkyl groups;
R¹⁰ and R¹¹ are independently absent, —H or —C₁-C₆ alkyl, or R¹⁰ and R¹¹ together form a —CH₂— or —CH₂CH₂— bridge between the atoms to which they are attached to form an azabicyclo group with ring A;
R¹² and R¹³ are independently absent, —H or —C₁-C₆ alkyl, or R¹² and R¹³ together form a —CH₂—, —CH₂CH₂—, or —CH₂CH₂CH₂— bridge between the atoms to which they are attached to form an azabicyclo group with ring A;
R¹⁴ is —H, —C₁-C₆ alkyl, —C(O)—Rⁱ, —C(O)O—C₁-C₆ alkyl, alkoxyalky, a 3-6 membered cycloalkyl ring, a 3-6 membered heterocycloalkyl ring having 1 heteroatom, a 5-9 membered spiro heterocycloalkyl group having 1 heteroatom, heteroarylalkyl, —S⁺(O—)(O)—CH₂CH₂—R^{j}, or 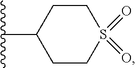 [SMILES: *C1CCS(=O)(=O)CC1]
wherein —C₁-C₆ alkyl is optionally substituted with one or more halo groups;
or R¹² and R¹⁴ together with the atoms to which they are attached form a 4-6 membered cycloalkyl ring;
Rⁱ is —H, —C₁-C₆ alkyl, —C₁-C₆ alkyl-NR^{k}R^{k′}, a 3-5 membered cycloalkyl ring, or a 3-6 membered heterocycloalkyl ring having 1 or 2 heteroatoms, wherein the alkyl of —C₁-C₆ alkyl-NR^{k}R^{k′} is optionally substituted with one or more of halo or —OH, and wherein the cycloalkyl or heterocycloalkyl ring is optionally substituted with one or more halo, —CF₃, —C₁-C₆ alkyl, —OH, oxo, or amine groups;
R^{j} is —N(CH₃)₂, —Si(CH₃)₃, a 3-5 membered cycloalkyl ring, or alkoxy; and
R^{k} and R^{k′} are each independently —H or —C₁-C₆ alkyl, wherein —C₁-C₆ alkyl is optionally substituted with one or more halo groups.

128. The compound of claim 127, wherein the compound is a compound of Formula IA: 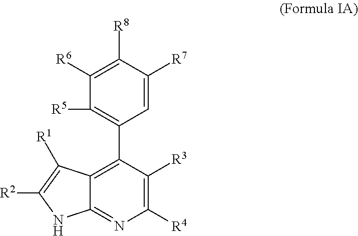
or a pharmaceutically acceptable salt thereof.

129. The compound of claim 127, wherein the compound is a compound of Formula IB 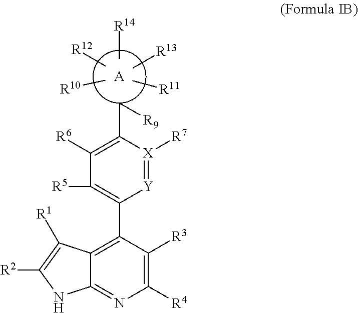
or a pharmaceutically acceptable salt thereof.

130. The compound of claim 127, wherein the compound is a compound of Formula IC: 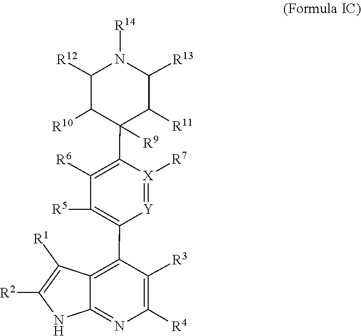
or a pharmaceutically acceptable salt thereof.

131. The compound of claim 127, wherein the compound is a compound of Formula ID 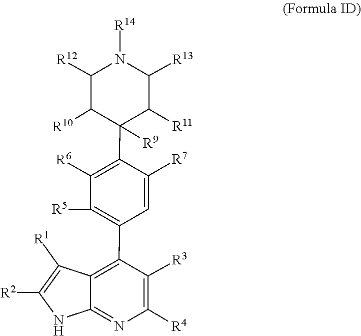
or a pharmaceutically acceptable salt thereof.

132. The compound of claim 127, wherein the compound is a compound of Formula IE 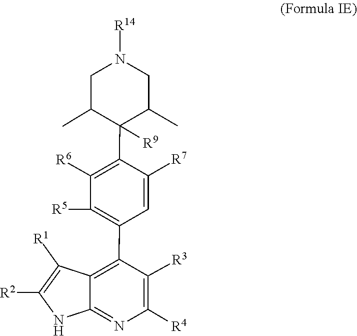
or a pharmaceutically acceptable salt thereof.

133. The compound of claim 127, wherein the compound is a compound of Formula IF 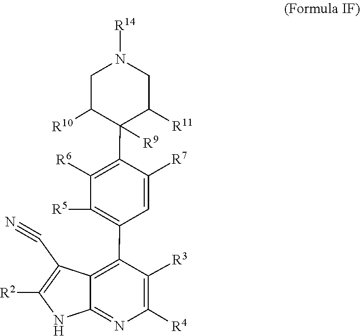
or a pharmaceutically acceptable salt thereof.

134. A pharmaceutical composition comprising the compound of claim 127 and a pharmaceutically acceptable carrier, excipient, or diluent.

135. A method of inhibiting one or more of large tumor suppressor kinase 1 (LATS1) and large tumor suppressor kinase 2 (LATS2) in a subject in need thereof, comprising administering to the subject a compound of claim 127.

136. A method of treating, preventing, or ameliorating a disease, disorder, or condition associated with LATS1 or LATS2 in a subject in need thereof comprising administering to the subject a therapeutically effective amount of a compound of claim 127.

137. A method of promoting ex vivo growth of a cell line or of a cell product comprising exposing the cell line or cell product to a compound of claim 127.

138. A method of accelerating tissue growth ex vivo comprising exposing the tissue to a compound of claim 127.

139. A compound of Formula II 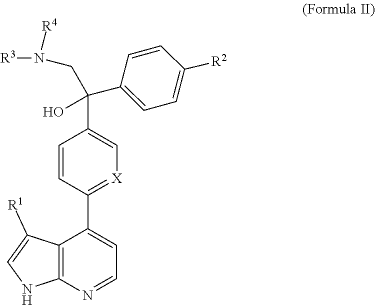
or a pharmaceutically acceptable salt thereof,
wherein
X is C or N;
R¹ is —H, halo, a 6-10 membered aryl ring, a 5-10 membered heteroaryl ring containing 1 or 2 heteroatoms, or —C(O)O—C₁-C₆ alkyl;
R² is —H or halo;
R³ is —H or —C₁-C₆ alkyl; and
R⁴ is —H, —C(O)—R^{a}, or 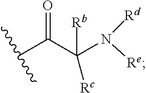
R^{a} is a 4-6 membered heteroaryl ring containing 1 or 2 heteroatoms, or —C₁-C₆ alkyl, wherein the —C₁-C₆ alkyl is optionally substituted with one or more of —OH, —P(O)(OH)₂;
R^{b} and R^{c} are independently selected from —H and —C₁-C₆ alkyl;
R^{d} is —H or —C₁-C₆ alkyl; and
R^{e} is —H, —C₁-C₆ alkyl, or —C(O)—CH₂NH₂.

140. The compound of claim 139, wherein the compound is a compound of Formula IIA 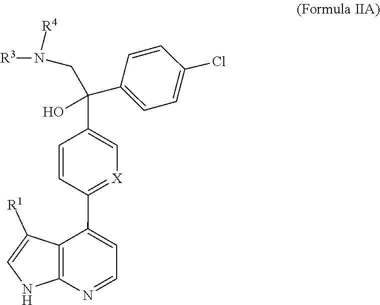
or a pharmaceutically acceptable salt thereof.

141. The compound of claim 139, wherein the compound is a compound of Formula IIB 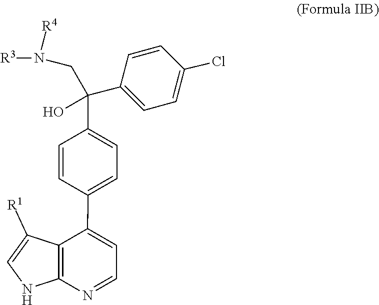
or a pharmaceutically acceptable salt thereof.

142. The compound of claim 139, wherein the compound is a compound of Formula IIC: 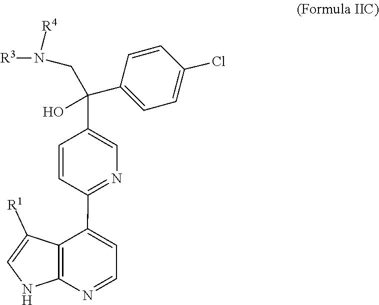
or a pharmaceutically acceptable salt thereof.

143. A pharmaceutical composition comprising the compound of claim 139 and a pharmaceutically acceptable carrier, excipient, or diluent.

144. A method of jointly inhibiting protein kinase B (AKT) and one or more of large tumor suppressor kinase 1 (LATS1) and large tumor suppressor kinase 2 (LATS2) in a subject in need thereof comprising administering to the subject a compound of claim 139.

145. A method of treating, preventing, or ameliorating a disease, disorder, or condition associated with LATS1, LATS2, or AKT in a subject in need thereof comprising administering to the subject a therapeutically effective amount of a compound of claim 139.

146. A method of promoting ex vivo growth of a cell line or of a cell product comprising exposing the cell line or cell product to a compound of claim 139.

147. A method of accelerating tissue growth ex vivo comprising exposing the tissue to a compound of claim 139.
